(19) United States
(12) Patent Application Publication
(10) Pub. No.: US 2025/0066318 A1
(43) Pub. Date: Feb. 27, 2025

(54) HETEROCYCLIC COMPOUNDS AS DYRK1A INHIBITORS

(71) Applicant: Prothena Biosciences Limited, Dublin (IE)

(72) Inventor: Stéphane DE LOMBAERT, Chester, CT (US)

(21) Appl. No.: 18/717,613

(22) PCT Filed: Dec. 9, 2022
(86) PCT No.: PCT/US2022/052439

§ 371 Date: Jun. 7, 2024

Related U.S. Application Data
(60) Provisional application No. 63/288,155, filed on Dec. 10, 2021.
(60) Provisional application No. 63/288,184, filed on Dec. 10, 2021.
(60) Provisional application No. 63/288,190, filed on Dec. 10, 2021.

(51) Int. Cl. C07D 401/04; A61K 31/415; A61K 31/4155; A61K 31/427; A61K 31/437; A61K 31/4439; A61K 31/444; A61K 31/454; A61K 31/4545; A61K 31/4725; A61K 31/496; A61K 31/497; A61K 31/4985; A61K 31/501; A61K 31/502; A61K 31/5025; A61K 31/506; A61K 31/517; A61K 31/519; A61K 31/5377; A61K 31/5415; C07D 231/40; C07D 277/46; C07D 401/12; C07D 403/12; C07D 405/14; C07D 413/04; C07D 417/04; C07D 417/06; C07D 417/12; C07D 417/14; C07D 471/04; C07D 471/14; C07D 487/04; C07D 491/08; C07D 493/04; C07D 493/08; C07D 519/00

(52) U.S. Cl. CPC C07D 401/04; A61K 31/415; A61K 31/4155; A61K 31/427; A61K 31/437; A61K 31/4439; A61K 31/444; A61K 31/454; A61K 31/4545; A61K 31/4725; A61K 31/496; A61K 31/497; A61K 31/4985; A61K 31/501; A61K 31/502; A61K 31/5025; A61K 31/506; A61K 31/517; A61K 31/519; A61K 31/5377; A61K 31/5415; C07D 231/40; C07D 277/46; C07D 401/12; C07D 403/12; C07D 405/14; C07D 413/04; C07D 417/04; C07D 417/06; C07D 417/12; C07D 417/14; C07D 471/04; C07D 471/14; C07D 487/04; C07D 491/08; C07D 493/04; C07D 493/08; C07D 519/00

(57) ABSTRACT

This disclosure provides compounds and pharmaceutically acceptable salts thereof, that inhibit Dual specificity tyrosine-phosphorylation-regulated kinase 1A (DYRK1A). These chemical entities are useful, e.g., for treating a condition, disease or disorder in which increased (e.g., excessive) DYRK1A activation contributes to the pathology and/or symptoms and/or progression of the condition, disease or disorder (e.g., a neurological disorder) in a subject (e.g., a human). This disclosure also provides compositions containing the same as well as methods of using and making the same.

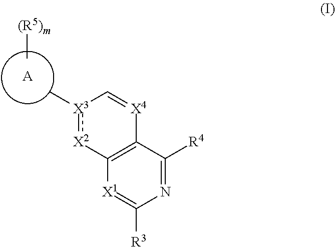

### TECHNICAL FIELD

This disclosure provides compounds and pharmaceutically acceptable salts thereof, that inhibit Dual specificity tyrosine-phosphorylation-regulated kinase 1A (DYRK1A). These compounds are useful, e.g., for treating a condition, disease or disorder in which increased (e.g., excessive) DYRK1A activation contributes to the pathology and/or symptoms and/or progression of the condition, disease or disorder (e.g., a neurological disorder in a subject (e.g., a human). This disclosure also provides compositions containing the same as well as methods of using and making the same.

### BACKGROUND

Dual-specificity tyrosine phosphorylation-regulated kinase 1A (DYRK1A) is a 763 amino acid, 85 kDa serine/threonine/tyrosine kinase located on chromosome 21 (21q22.2). DYRK1A possesses catalytic activity that is regulated by autophosphorylation of a tyrosine residue (Y321) which results in constitutively active serine/threonine kinase activity. See Abbassi, et al., Pharmacology & Therapeutics, 151, 87-98 (2015). Since DYRK1A is constitutively active, its activity is dosage dependent. Thus, both elevated levels and depressed levels of DYRK1A, (relative to wild-type levels) have been shown to lead to neurological impairment. See Duchon and Herault, Front Behav. Neurosci. 10, 104-104 (2016). DYRK1A is also a member of a large family of CMGC kinases, which include cyclin-dependent kinases (CDKs), mitogen-activated protein kinases (MAPKs), glycogen synthase kinases (GSKs), and CDC-like kinases (CLKs).

DYRK1A additionally has been shown to have a role in cell cycle regulation, at least in part by phosphorylating (and thus inhibiting) the nuclear factor of activated T cells (NFAT) family of transcription factors. Additionally, over 20 substrates of DYRK1A have been identified, including cell signaling, chromatin modulation, gene expression, alternative splicing, cytoskeletal, and synaptic function. See Abassi, et al, (2016). DYRK1A dysregulation is implicated in various disease states such as Alzheimer's disease, autism, and Down syndrome. In some cases, novel mutations in DYRK1A have been associated with autism phenotypes. See e.g., Dang, et al., Molecular Psychiatry, 23, 747-758 (2018).

DYRK1A is also known to play an important role in brain development. For example, reduced DYRK1A activity (such has having a single copy of loss of function mutation) during neural development results in intellectual disability phenotypes. Conversely, trisomy 21 in Down syndrome individuals is associated with a triplication of the DYRK1A gene, which results in elevated DYRK1A activity. DYRK1A is located on chromosome 21, specifically within the “Down syndrome critical region” a portion of chromosome 21 that includes genes particularly relevant for developing Down syndrome phenotypes. As a result, individuals with Down syndrome have three copies of DYRK1A, and since DYRK1A is dosage sensitive, the elevated levels of DYRK1A in such individuals markedly affects the localization and function of the DYRK1A protein. The expression of DYRK1A is also elevated in the CNS in individuals with neurodegenerative diseases, such as Parkinson's disease, Pick's disease, and Alzheimer's disease.

Moreover, approximately 50% of individuals with Down syndrome ultimately develop Alzheimer's disease, with symptoms generally beginning between the ages of 40 and 60. DYRK1A phosphorylates amyloid precursor protein (APP) which promotes the production of pathogenic amyloid-β peptide (Aβ). Dyrk1A also phosphorylates tau both directly and indirectly (see Abassi, et al, (2016)). Both amyloid-β and tau pathologies are associated with Down syndrome phenotypes.

Normalization of DYRK1A gene dosage by crossing Ts65Dn mice (DS model) with DYRK1A knockout mice mice reverses many Azlheimer's-like phenotypes. See García-Cerro et al., 2017. In individuals with Down Syndrome, DYRK1A mRNA levels, protein levels, and kinase activity are increased by ˜50%, reflecting the number of gene copies. See Liu et al., 2008; see also Wegiel et al., 2011.

Because no treatment is available for these neurological disorders, the prognosis for individuals with, for example, Alzheimer's disease is poor. This can be particularly devastating because Alzheimer's disease is responsible for a sharp decline in survival in individuals with Down syndrome that are over 45 years old. Only about 25% of those with Down syndrome live more than 60 years, and most of those have developed Alzheimer's disease.

Across all individuals, dementia remains a significant leading unmet medical need and a costly burden on public health. Currently, 1 in 3 seniors develops dementia, and about 70% of dementia cases are attributed to Alzheimer's disease. Some 11% of Americans over age 65 has AD, which constitutes over 6.2 million in 2021. This figure is projected to exceed 12 million in 2050 (www.Alz.org).

Presently, no therapies have been approved to treat Alzheimer's disease associated with Down syndrome, which represents a significant unmet medical need. Some DYRK1A inhibitors have been tested in vitro or in animal preclinical models to treat Alzheimer's disease or Down syndrome, however, since DYRK1A is a member of the highly conserved CMGC family of kinases, identifying compounds that selectively target DYRK1A has proved challenging. Thus, there remains a need to identify DYRK1A inhibitors to treat Down syndrome, Alzheimer's disease, Alzheimer's disease associated with Down syndrome, and other neurodegenerative and neurological diseases.

### SUMMARY

Some embodiments provide a compound of Formula (I-O):

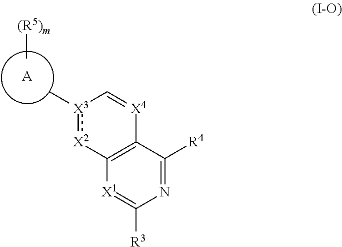

or a pharmaceutically acceptable salt thereof, wherein:
each dashed line represents a single bond or a double bond;
X¹ is CR¹ or N;
X² is CR², C(═O), or N;
X³ is C or N; provided that when X² is C(═O), X³ is N;
X⁴ is CH or N;
Ring A is phenyl or 5-6 membered heteroaryl;
R¹ is hydrogen, halogen, cyano, hydroxyl, 3-10 membered heterocyclyl, C1-C6 alkyl optionally substituted with 3-6 membered heterocyclyl optionally substituted with —C(═O)C1-C6 alkyl or —C(═O)OR^{A}, C1-C6 alkoxy, C1-C6 haloalkyl, C1-C6 haloalkoxy, —C(═O)-3-6 membered heterocyclyl optionally substituted with C1-C6 alkyl or halogen, or —OR^{B};
R^{B} is a 3-6 membered heterocyclyl, a 5-6 membered heteroaryl, a C6-C10 aryl, or a C3-C6 cycloalkyl each optionally substituted with 1-3 independently selected halogen, C1-C6 alkyl, C1-C6 haloalkyl, C1-C6 alkoxy, C1-C6 haloalkoxy, cyano, hydroxy, —C(═O)OH, —C(═O)C1-C6 alkyl, —S(O₂)—C1-C6 alkyl, or —NR^{C}R^{D};
R² is hydrogen, halogen, C1-C6 alkyl, C1-C6 alkoxy, C1-C6 haloalkoxy, —C(═O)-3-6 membered heterocyclyl, —NH—C3-C6 cycloalkyl-C(═O)OR^{A}, or —O—C3-C6 cycloalkyl-C(═O)OR^{A};
R³ is hydrogen, halogen, hydroxyl, C1-C6 alkyl, C1-C6 alkoxy, C1-C6 haloalkyl, C1-C6 haloalkoxy, cyano, C3-C6 cycloalkyl, —X—R^{G}, or

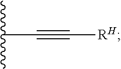

R⁴ is hydrogen or C1-C6 alkyl;
R⁵ is hydrogen, C1-C6 alkyl optionally substituted with 3-6 membered heterocyclyl; —X—R^{E}; —C3-C6 cycloalkyl-C(═O)OR^{A}; or

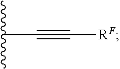

or
R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a (i) C6-C10 aryl optionally substituted with a 3-10 membered heterocyclyl optionally substituted with 1-2 independently selected C1-C6 alkyl or —C(═O)OR′; (ii) a 3-6 membered heterocyclyl; or a (iii) a 5-6 membered heteroaryl optionally substituted with 1-2 substituents independently selected from halogen, C1-C6 alkyl, C1-C6 haloalkyl, C1-C6 alkoxy, C1-C6 haloalkoxy, 3-10 membered heterocyclyl optionally substituted with 1-2 independently selected C1-C6 alkyl or C(═O)OR′, and 5-6 membered heteroaryl optionally substituted with C1-C6 alkyl;
R^{E} is a 3-10 membered heterocyclyl, a 5-6 membered heteroaryl, or a C3-C6 cycloalkyl each optionally substituted with 1-3 independently selected C1-C6 alkyl optionally substituted with C1-C6 alkoxy, NR^{I}R^{J}, or —C(═O)OH;
R^{F} is a 3-6 membered heterocyclyl, a 5-6 membered heteroaryl, or a C3-C6 cycloalkyl each optionally substituted with C1-C6 alkyl; or a C2-C6 alkynyl optionally substituted with hydroxy;
R^{G} is a 3-6 membered heterocyclyl or a C3-C6 cycloalkyl each optionally substituted with 1-3 independently selected C1-C6 alkyl, —C(═O)C1-C6 alkyl, —C(═O)OH, or NR^{C}R^{D};
R^{H} is a C1-C6 alkoxy, C1-C6 alkyl optionally substituted with hydroxy, or a 3-6 membered heterocyclyl optionally substituted with C1-C6 alkyl;
X is —NH—, —NH(C═O)—, —NHC(═O)O—, —O—, —(C═O)— or CH₂;
each R^{A}, R^{C}, R^{D}, R¹, and R^{J} is independently selected from hydrogen and C1-C6 alkyl; and
m is 0, 1, or 2.

Some embodiments provide a compound of Formula (I):

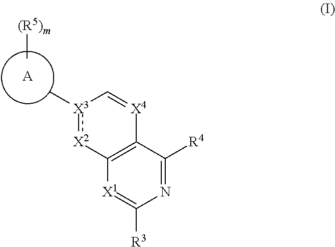

or a pharmaceutically acceptable salt thereof, wherein.
each dashed line represents a single bond or a double bond;
X¹ is CR¹ or N;
X² is CR², C(═O), or N;
X³ is C or N; provided that when X² is C(═O), X³ is N;
X⁴ is CH or N;
Ring A is phenyl or 5-6 membered heteroaryl;
R¹ is hydrogen, halogen, cyano, 3-10 membered heterocyclyl, C1-C6 alkyl optionally substituted with 3-6 membered heterocyclyl optionally substituted with —C(═O)C1-C6 alkyl or —C(═O)OR^{A}, C1-C6 alkoxy, —C(═O)-3-6 membered heterocyclyl optionally substituted with C1-C6 alkyl, or —OR^{B};
R^{B} is a 3-6 membered heterocyclyl, a 5-6 membered heteroaryl, a C6-C10 aryl, or a C3-C6 cycloalkyl each optionally substituted with 1-3 independently selected halogen, C1-C6 alkyl, C1-C6 haloalkyl, C1-C6 alkoxy, cyano, hydroxy, —C(═O)OH, —C(═O)C1-C6 alkyl, —S(O₂)—C1-C6 alkyl, or —NR^{C}R^{D};
R² is hydrogen, halogen, C1-C6 alkyl, C1-C6 alkoxy, —C(═O)-3-6 membered heterocyclyl, —NH—C3-C6 cycloalkyl-C(═O)OR^{A}, or —O—C3-C6 cycloalkyl-C(═O)OR^{A};

R³ is hydrogen, halogen, C1-C6 alkyl, cyano, C3-C6 cycloalkyl, —X—R^{G}, or

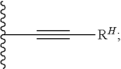

R⁴ is hydrogen or C1-C6 alkyl;
R⁵ is hydrogen, C1-C6 alkyl optionally substituted with 3-6 membered heterocyclyl; —X—R^{E}; —C3-C6 cycloalkyl-C(═O)OR^{A}; or

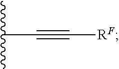

or
R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a (i) C6-C10 aryl optionally substituted with a 3-10 membered heterocyclyl optionally substituted with 1-2 independently selected C1-C6 alkyl or —C(═O)OR′; (ii) a 3-6 membered heterocyclyl; or a (iii) a 5-6 membered heteroaryl optionally substituted with 1-2 substituents independently selected from C1-C6 alkyl, C1-C6 haloalkyl, C1-C6 alkoxy, 3-10 membered heterocyclyl optionally substituted with 1-2 independently selected C1-C6 alkyl or C(═O)OR′, and 5-6 membered heteroaryl optionally substituted with C1-C6 alkyl;
R^{E} is a 3-10 membered heterocyclyl, a 5-6 membered heteroaryl, or a C3-C6 cycloalkyl each optionally substituted with 1-3 independently selected C1-C6 alkyl optionally substituted with C1-C6 alkoxy, NR^{I}R^{J}, or —C(═O)OH;
R^{F} is a 3-6 membered heterocyclyl, a 5-6 membered heteroaryl, or a C3-C6 cycloalkyl each optionally substituted with C1-C6 alkyl; or a C2-C6 alkynyl optionally substituted with hydroxy;
R^{G} is a 3-6 membered heterocyclyl or a C3-C6 cycloalkyl each optionally substituted with 1-3 independently selected C1-C6 alkyl, —C(═O)C1-C6 alkyl, —C(═O)OH, or NR^{C}R^{D};
R^{H} is a C1-C6 alkyl optionally substituted with hydroxy or a 3-6 membered heterocyclyl optionally substituted with C1-C6 alkyl;
X is —NH—, —NH(C═O)—, —NHC(═O)O—, —O—, —(C═O)— or CH₂;
each R^{A}, R^{C}, R^{D}, R^{I}, and R^{J} is independently selected from hydrogen and C1-C6 alkyl; and
m is 0, 1, or 2.

Some embodiments provide a compound of Formula (II):

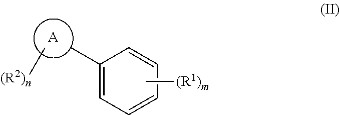

or a pharmaceutically acceptable salt thereof, wherein:
Ring A is a 5-14 membered heteroaryl or a 5-14 membered heterocyclyl;
each R¹ is independently halogen, hydroxyl, cyano, C1-C6 alkyl, C1-C6 alkoxy, —C(═O)OR^{A}, —NR^{B}R^{C}, and —C(═O)NR^{B}R^{C};
each R² is independently —C(═O)OR^{D}, C1-C6 alkyl, C2-C6 alkynyl optionally substituted with 4-8 membered heterocyclyl optionally substituted with C1-C6 alkyl, —C(═O)-phenyl, —(C1-C6 alkyl)-phenyl, —(C1-C6 alkyl)-4-10 membered heterocyclyl optionally substituted with C1-C6 alkyl, 4-10 membered heterocyclyl optionally substituted with C1-C6 alkyl or —CO₂C1-C6 alkyl, phenyl optionally substituted with cyano or fluoro, —NHC(═O)R^{E}, 5-6 membered heteroaryl optionally substituted with C1-C6 alkoxy,
m is 1, 2, or 3;
n is 0, 1, 2, or 3;
each R^{A}, R^{B}, R^{C}, and R^{D} is independently hydrogen or C1-C6 alkyl; and
each R^{E} is independently C3-C6 cycloalkyl, 4-8 membered heterocyclyl optionally substituted with C1-C6 alkyl, or 5-6 membered heteroaryl optionally substituted with C1-C6 alkyl.

Some embodiments provide a compound of Formula (III):

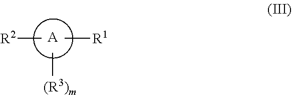

or a pharmaceutically acceptable salt thereof:
Ring A is 5-6 membered heteroaryl or 5-6 membered heterocyclyl;
R¹ is —NHC(═O)(C1-C6 alkylene)ₙR^{A}, phenyl optionally substituted with —NR^{F}R^{G}, -Q-R^{C}, or

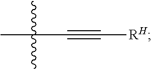

R² is C3-C6 cycloalkyl optionally substituted with —CO₂R^{B}, 5-10 membered heteroaryloxy, —(C1-C6 alkylene)ₚ-5-10 membered heteroaryl optionally substituted with C1-C6 alkyl, cyano, or 4-6 membered heterocyclyl; —(C1-C6 alkylene)ₜ-phenyl optionally substituted with cyano or —NR^{D}R^{E}; 4-6 membered heterocyclyl optionally substituted with C1-C6 alkyl;
R³ is C1-C6 alkyl;
R^{A} is 4-6 membered heterocyclyl optionally substituted with C1-C6 alkyl, or 5-10 membered heteroaryl optionally substituted with C1-C6 alkoxy or C1-C6 alkyl,
R^{B} is hydrogen or C1-C6 alkyl;
R^{C} is 4-10 membered heterocyclyl, 5-10 membered heteroaryl, or phenyl optionally substituted with —(C1-C6 alkylene)-NR^{D}R^{E};
R^{D}, R^{E}, and R^{F} are independently hydrogen, C1-C6 alkyl, or C3-C6 cycloalkyl;
R^{G} is hydrogen, C1-C6 alkyl, —C(═O)—C1-C6 alkyl, or —C(═O)—C3-C6 cycloalkyl;
R^{H} is 4-6 membered heterocyclyl optionally substituted with 1-2 independently selected C1-C6 alkyl;
Q is C1-C6 alkylene, NH, or O;
m is 0 or 1;
n is 0 or 1;
p is 0 or 1; and
t is 0 or 1.

Also provided herein is a pharmaceutical composition comprising a compound of Formula (I), or a pharmaceutically acceptable salt thereof, and a pharmaceutically acceptable carrier.

Provided herein is a method for treating a neurological disorder in a subject in need thereof, the method comprising administering to the subject a therapeutically effective amount of a compound of Formula (I), (II), or (III), or a pharmaceutically acceptable salt of any of the foregoing, or a pharmaceutical composition as provided herein.

Also provided herein is a method for treating a neurological disorder in a subject in need thereof, the method comprising (a) determining that the neurological disorder is associated with a dysregulation of a DYRK1A gene, a DYRK1A protein, or expression or activity or level of any of the same; and (b) administering to the subject a therapeutically effective amount of a compound of Formula (I), (II), or (III), or a pharmaceutically acceptable salt of any of the foregoing, or a pharmaceutical composition as provided herein.

Provided herein is a method of treating a DYRK1A-associated disease or disorder in a subject, the method comprising administering to a subject identified or diagnosed as having a DYRK1A-associated disease or disorder a therapeutically effective amount of a compound of Formula (I), (II), or (III), or a pharmaceutically acceptable salt of any of the foregoing, or a pharmaceutical composition as provided herein.

This disclosure also provides a method of treating a DYRK1A-associated neurological disorder in a subject, the method comprising: determining that the neurological disorder in the subject is a DYRK1A-associated disease or disorder; and administering to the subject a therapeutically effective amount of a compound of Formula (I), (II), or (III), or a pharmaceutically acceptable salt of any of the foregoing, or a pharmaceutical composition as provided herein.

Further provided herein is a method of treating a DYRK1A-associated neurological disorder in a subject, the method comprising administering to a subject identified or diagnosed as having a DYRK1A-associated neurological disorder a therapeutically effective amount of a compound of Formula (I), (II), or (III), or a pharmaceutically acceptable salt of any of the foregoing, or a pharmaceutical composition as provided herein.

This disclosure also provides a method of treating a DYRK1A-associated neurological disorder in a subject, the method comprising: determining that the neurological disorder in the subject is a DYRK1A-associated neurological disorder; and administering to the subject a therapeutically effective amount of a compound of Formula (I), (II), or (III), or a pharmaceutically acceptable salt of any of the foregoing, or a pharmaceutical composition as provided herein.

Provided herein is a method of treating a subject, the method comprising administering a therapeutically effective amount of a compound of Formula (I), (II), or (III), or a pharmaceutically acceptable salt of any of the foregoing, or a pharmaceutical composition as provided herein, to a subject having a clinical record that indicates that the subject has a dysregulation of a DYRK1A gene, a DYRK1A protein, or expression or activity or level of any of the same.

This disclosure also provides a method for inhibiting DYRK1A in a mammalian cell, the method comprising contacting the mammalian cell with a therapeutically effective amount of a compound of Formula (I), (II), or (III), or a pharmaceutically acceptable salt of any of the foregoing.

The details of one or more embodiments of this disclosure are set forth in the accompanying drawings and the description below. Other features and advantages of the present disclosure will be apparent from the description and the claims.

### DETAILED DESCRIPTION

Dual-specificity tyrosine phosphorylation-regulated kinase 1A (DYRK1A) is a member of the dual-specificity tyrosine phosphorylation regulated kinase (DYRK) family, which is also part of the larger CGMC family of kinases. DYRK1A is a 763 amino acid, 85 kDa serine/threonine kinase located on chromosome 21. DYRK1A contains a nuclear targeting signal sequence, a protein kinase domain, a leucine zipper motif, and a highly conservative 13-consecutive-histidine repeat. Alternative splicing DYRK1A generates several transcript variants differing from each other either in either the 5′ untranslated region or in the 3′ coding region resulting in at least five different isoforms.

DYRK1A possesses catalytic activity that is regulated by autophosphorylation of a tyrosine residue (Y321) which results in constitutively active serine/threonine kinase activity. Since DYRK1A is constitutively active, its activity is dosage dependent. Thus, both elevated levels and depressed levels of DYRK1A (relative to wild-type levels) have been shown to lead to neurological impairment.

DYRK1A displays a broad substrate spectrum (e.g., broad range of targets) including splicing factors, synaptic proteins, and transcription factors. It is ubiquitously expressed in all mammalian tissues and cells, although at different levels, with particularly high levels in embryonic and adult brain tissues. The human DYRK1A gene is a candidate gene to treat several Down syndrome characteristics, including intellectual impairment and Alzheimer's disease associated with Down syndrome, due to its localization in the Down syndrome critical region on chromosome 21 and its role in brain function. Notably, Drosophila with deleterious mutations in the ortholog of DYRK1A (“Minibrain”) have a reduced number of neurons in their central nervous system. Likewise, mice heterozygous for a disrupted allele of the Dyrk1a gene exhibit decreased viability, behavioral alterations, and delayed growth. Fotaki, et al., Mol CellBiol., 22(18): 6636-6647 (2014).

The identification of hundreds of genes deregulated by DYRK1A overexpression and numerous cytosolic, cytoskeletal and nuclear proteins, including transcription factors, phosphorylated by DYRK1A, indicates that DYRK1A overexpression is central for the deregulation of multiple pathways in the developing and aging brain of individuals with Down syndrome. Identifying DYRK1A cell signaling or transduction pathways can lead to a better understanding of how DYRK1A overexpression (or under expression) leads to the various disease states in which it is known to be involved. Specifically, DYRK1A is known to be active in activated PI3K/Akt signaling, a pathway largely involved in neuronal development, growth, and survival. DYRK1A is also known to be active in ASK1/JNK1 activity and inhibitors of DYRK1A may induce neuronal death and apoptosis. DYRK1A is also known to phosphorylate p53 during embryonic brain development, and inhibitors of DYRK1A can prevent neuronal proliferation alteration. DYRK1A also phosphorylates synaptic proteins Amph 1, Dynamin 1, and Synaptojanin, which are involved in the regulation of endocytosis and inhibitors of DYRK1A can retain synaptic plasticity through preventing alteration of the number, size, and morphology of dendritic spines. DYRK1A also phosphorylates inhibit presenilin 1, the catalytic sub-unit of γ-secretase. Ryu, et al., J Neurochem., 115(3): 574-84 (2010).

DYRK1A overexpression leads to structural and functional alterations including intellectual disability and dementia, e.g., Alzheimer's disease. In particular, genes involved in learning disorders, synaptic flexibility changes, memory loss, and abnormal cell cycles, result in neuropathological symptoms similar to dementia associated with Alzheimer's disease. DYRK1A can also affect the proliferation and differentiation of neuronal progenitors, thus influencing neurogenesis and brain growth. It can also affect neurotransmission and dendritic spine formation through its interaction with synaptic proteins and the cytoskeleton.

One potential source of treatment are inhibitors of DYRK1A. Inhibitors that can normalize DYRK1A levels in Down syndrome may improve synaptic plasticity and delay the onset of Alzheimer's disease pathology, including tau hyperphosphorylation. Therefore, inhibiting DYRK1A activity in individuals with Down syndrome might counteract the phenotypic effects of its overexpression and is a potential avenue for the treatment of such developmental defects and prevention and/or mitigation of age-associated neurodegeneration, including Alzheimer's disease associated with Down syndrome. Studies have shown that inhibition of overexpressed DYRK1A resulted in normal DYRK1A levels and been found to improve cognitive and behavioral deficits in transgenic models. See, e.g., Stringer, et al., Mol Genet Genomic Med, 5, 451-465 (2017) and Feki and Hibaoui, Brain Sci, 8, 187 (2018). However, despite promising results there is considerable variation across studies in terms of outcomes. Discrepancies were attributed to differences in model, dose, route of administration, the composition of the inhibitor, and timing of administration.

Epigallocatechin gallate (EGCG) is the primary flavonoid of green tea and has been investigated for its therapeutic effects, which include anti-oxidative, anti-inflammatory, anti-cancer, anti-infective and neuroprotective activity. See, Bhat, et al. Towards the discovery of drug-like epigallocatechin gallate analogs as Hsp90 inhibitors, BioorgMed Chem Lett, 24, 2263-2266 (2014). EGCG is a non-ATP competitive DYRK1A inhibitor and studies have shown that green tea extract comprising 41% EGCG were able to alleviate cognitive decline seen in transgenic mice over expressing DYRK1A. ECGC has also been shown to improve memory recognition and working memory. However, ECGC is not significantly selective and has numerous off-target effects, thus reducing its potential long-term use.

SM07883 is an orally bioavailable (% F 92% in mice, 109% in monkey), BBB penetrant, DYRK1A inhibitor (IC50 1.6 nM) that also shows potent inhibition for DYRK1B, CLK4, and GSK3β in kinase assays. It was found to protect against tau hyperphosphorylation in mouse models. SM07883 was tested for treatment of Alzheimer's disease in a phase 1 study in Australia (ACTRN12619000327189). However, according to the study description page at www.anzctr.org.au, the date of last data collection was in May 2019 and no results have been published for the trial.

This disclosure provides compounds of Formula (I), (II), and (III), and pharmaceutically acceptable salts of any of the foregoing, that inhibit Dual specificity tyrosine-phosphorylation-regulated kinase 1A (DYRK1A). These chemical entities are useful, e.g., for treating a condition, disease or disorder in which increased (e.g., excessive) DYRK1A activation contributes to the pathology and/or symptoms and/or progression of the condition, disease or disorder (e.g., a neurological disorder in a subject (e.g., a human). This disclosure also provides compositions containing the same as well as methods of using and making the same.

### Definitions

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control.

The term “compound,” as used herein is meant to include all stereoisomers, geometric isomers, tautomers, and isotopically enriched variants of the structures depicted. Compounds herein identified by name or structure as one particular tautomeric form are intended to include other tautomeric forms unless otherwise specified. For example, if quinazolin-4-ol is encompassed by a claim or embodiment, then quinazolin-4(3H)-one is also covered by the claim or embodiment (see below).

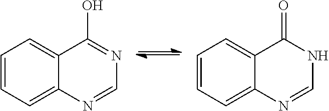 [SMILES: Oc1ncnc2ccccc12 ; O=c1[nH]cnc2ccccc12]

It will be appreciated that certain compounds provided herein may contain one or more centers of asymmetry and may therefore be prepared and isolated in a mixture of isomers such as a racemic mixture, or in an enantiomerically pure form. Unless otherwise indicated, when a disclosed compound is named or depicted by a structure without specifying the stereochemistry (e.g., a “flat” structure) and has one or more chiral centers, it is understood to represent all possible stereoisomers of the compound.

The term “about” when referring to a number or a numerical range means that the number or numerical range referred to is an approximation, for example, within experimental variability and/or statistical experimental error, and thus the number or numerical range may vary up to +10% of the stated number or numerical range.

The term “acceptable” with respect to a formulation, composition or ingredient, as used herein, means having no persistent detrimental effect on the general health of the subject being treated.

The term “inhibit” or “inhibition of” means to reduce by a measurable amount, or to prevent entirely (e.g., 100% inhibition).

The term “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, a therapeutically effective amount” for therapeutic uses is the amount of the composition comprising a compound as disclosed herein required to provide a clinically significant decrease in disease symptoms. An appropriate “therapeutically effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The term “halogen” refers to fluoro (F), chloro (C1), bromo (Br), or iodo (I).

The term “oxo” refers to a divalent doubly bonded oxygen atom (i.e., “═O”). As used herein, oxo groups are attached to carbon atoms to form carbonyls.

The term “hydroxyl” refers to an —OH radical.

The term “cyano” refers to a —CN radical.

The term “alkyl” refers to a saturated acyclic hydrocarbon radical that can be straight chain or branched chain, containing the indicated number of carbon atoms. For example, C1-C10 indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein. A “C0” alkyl refers to a bond, e.g., phenyl-(C0 alkyl)-OH corresponds to phenol.

The term “haloalkyl” refers to an alkyl group in which one or more hydrogen atoms is/are replaced with an independently selected halogen.

The term “alkoxy” refers to an —O-alkyl radical (e.g., —OCH₃).

The term “aryl” refers to a 6-20 carbon atom monocyclic, bicyclic, or tricyclic group wherein at least one ring in the system is aromatic. Examples of aryl groups include phenyl, naphthyl, tetrahydronaphthyl, and the like.

The term “cycloalkyl” as used herein refers to cyclic hydrocarbon groups having the indicated number of carbon atoms, e.g., 3 to 20 ring carbons (C3-C20), 3 to 16 ring carbons (C3-C16), 3-10 ring carbons (C3-C10), or 3-6 ring carbons (C3-C6). Cycloalkyl groups are saturated or partially unsatured (but not aromatic). Examples of cycloalkyl groups include, without limitation, cyclopropyl, cyclobutyl, cyclopentyl, cyclopentenyl, cyclohexyl, cyclohexenyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

The term “heteroaryl”, as used herein, means a monocyclic, bicyclic, or tricyclic group having 5 to 20 ring atoms (5-20 membered heteroaryl), such as 5, 6, 9, 10, or 14 ring atoms; wherein at least one ring in the system contains one or more heteroatoms independently selected from the group consisting of N, O, and S and at least one ring in the system is aromatic (but does not have to be a ring which contains a heteroatom, e.g. tetrahydroisoquinolinyl, e.g., tetrahydroquinolinyl). Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. Heteroaryl groups can also include oxidized carbon (C═O), nitrogen (N—O), and/or sulfur atoms (S═O and S(═O)₂), as well as imino (═NH) groups. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

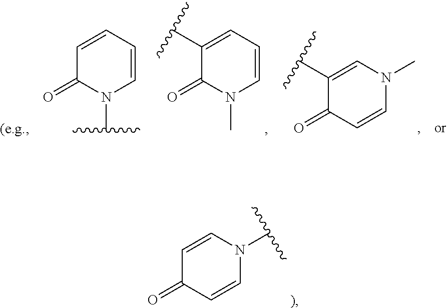

pyrimidone

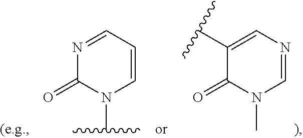

pyridazinone

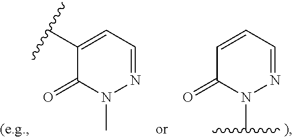

pyrazinone

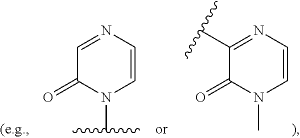

and imidazolone

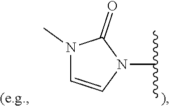

wherein each ring nitrogen adjacent to a carbonyl is tertiary (i.e., the oxo group (i.e., “═O”) herein is a constituent part of the heteroaryl ring).

The term “heterocyclyl” refers to monocyclic, bicyclic, or tricyclic saturated or partially unsaturated ring systems with 3-16 ring atoms (e.g., 5-8 membered monocyclic, 8-12 membered bicyclic, or 11-14 membered tricyclic ring systems) having 1-3 heteroatoms if monocyclic, 1-6 heteroatoms if bicyclic, or 1-9 heteroatoms if tricyclic. The heteroatoms are selected from the group consisting of O, N, or S (e.g., carbon atoms and 1-3, 1-6, or 1-9 heteroatoms of N, O, or S if monocyclic, bicyclic, or tricyclic, respectively), wherein one or more ring atoms may be substituted by 1-3 oxo (forming, e.g., a lactam) and one or more N or S atoms may be substituted by 1-2 oxido (forming, e.g., an N-oxide, an S-oxide, or an S,S-dioxide), valence permitting; and wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, tetrahydropyridyl, dihydropyrazinyl, dihydropyridyl, dihydropyrrolyl, dihydrofuranyl, dihydrothiophenyl, and the like. Heterocyclyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, examples of aromatic rings include: benzene, pyridine, pyrimidine, pyrazine, pyridazine, pyridone, pyrrole, pyrazole, oxazole, thioazole, isoxazole, isothiazole, and the like.

The term “saturated” as used in this context means only single bonds present between constituent atoms.

As used herein, when a ring is described as being “partially unsaturated”, it means said ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, cycloalkyl, and the like described herein) containing a sufficient number of ring atoms to form bicyclic or higher order ring systems (e.g., tricyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

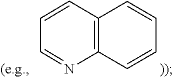

(ii) a single ring atom (spiro-fused ring systems)

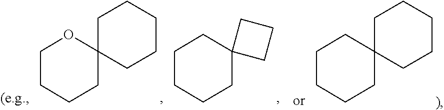

or (iii) a contiguous array of ring atoms (bridged ring systems having all bridge lengths >0)

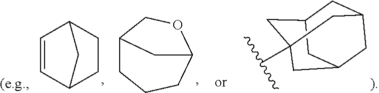

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

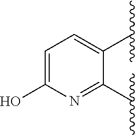 [SMILES: *c1ccc(O)nc1*]

encompasses the tautomeric form containing the moiety:

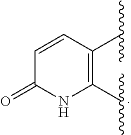 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

Dashed lines in chemical structures, for example, [symbol] and [symbol] represent single or double bonds. One skilled in the art understands that, in this structure,

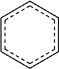

for example, the maximum number of double bonds is three.

### Compounds of Formula (I)

The substituent groups used in this section (e.g., R¹, R², and the like) refer solely to the groups in Formula (I).

Some embodiments provide a compound of Formula (I):

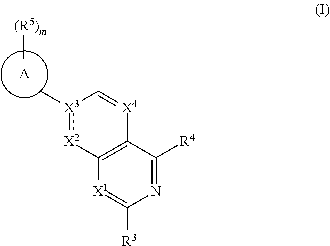

or a pharmaceutically acceptable salt thereof, wherein:
each dashed line represents a single bond or a double bond;
X¹ is CR¹ or N;
X² is CR², C(═O), or N;
X³ is C or N; provided that when X² is C(═O), X³ is N;
X⁴ is CH or N;
Ring A is phenyl or 5-6 membered heteroaryl;
R¹ is hydrogen, halogen, cyano, 3-10 membered heterocyclyl, C1-C6 alkyl optionally substituted with 3-6 membered heterocyclyl optionally substituted with —C(═O)C1-C6 alkyl or —C(═O)OR^{A}, C1-C6 alkoxy, —C(═O)-3-6 membered heterocyclyl optionally substituted with C1-C6 alkyl, or —OR^{B};
R^{B} is a 3-6 membered heterocyclyl, a 5-6 membered heteroaryl, a C6-C10 aryl, or a C3-C6 cycloalkyl each optionally substituted with 1-3 independently selected halogen, C1-C6 alkyl, C1-C6 haloalkyl, C1-C6 alkoxy, cyano, hydroxy, —C(═O)OH, —C(═O)C1-C6 alkyl, —S(O₂)—C1-C6 alkyl, or —NR^{C}R^{D}.

R² is hydrogen, halogen, C1-C6 alkyl, C1-C6 alkoxy, —C(═O)-3-6 membered heterocyclyl, —NH—C3-C6 cycloalkyl-C(═O)OR^{A}, or —O—C3-C6 cycloalkyl-C(═O)OR^{A};

R³ is hydrogen, halogen, C1-C6 alkyl, cyano, C3-C6 cycloalkyl, —X—R^{G}, or

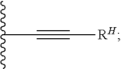

R⁴ is hydrogen or C1-C6 alkyl;
R⁵ is hydrogen, C1-C6 alkyl optionally substituted with 3-6 membered heterocyclyl; —X—R^{E}; —C3-C6 cycloalkyl-C(═O)OR^{A}; or

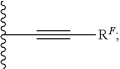

or
R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a (i) C6-C10 aryl optionally substituted with a 3-10 membered heterocyclyl optionally substituted with 1-2 independently selected C1-C6 alkyl or —C(═O)OR′; (ii) a 3-6 membered heterocyclyl; or a (iii) a 5-6 membered heteroaryl optionally substituted with 1-2 substituents independently selected from C1-C6 alkyl, C1-C6 haloalkyl, C1-C6 alkoxy, 3-10 membered heterocyclyl optionally substituted with 1-2 independently selected C1-C6 alkyl or C(═O)OR′, and 5-6 membered heteroaryl optionally substituted with C1-C6 alkyl;
R^{E} is a 3-10 membered heterocyclyl, a 5-6 membered heteroaryl, or a C3-C6 cycloalkyl each optionally substituted with 1-4 independently selected C1-C6 alkyl optionally substituted with C1-C6 alkoxy, NR^{I}R^{J}, or —C(═O)OH;
R^{F} is a 3-6 membered heterocyclyl, a 5-6 membered heteroaryl, or a C3-C6 cycloalkyl each optionally substituted with C1-C6 alkyl; or a C2-C6 alkynyl optionally substituted with hydroxy;
R^{G} is a 3-6 membered heterocyclyl or a C3-C6 cycloalkyl each optionally substituted with 1-3 independently selected C1-C6 alkyl, —C(═O)C1-C6 alkyl, —C(═O)OH, or NR^{C}R^{D};
R^{H} is a C1-C6 alkyl optionally substituted with hydroxy or a 3-6 membered heterocyclyl optionally substituted with C1-C6 alkyl;
X is —NH—, —NH(C═O)—, —NHC(═O)O—, —O—, —(C═O)— or CH₂;
each R^{A}, R^{C}, R^{D}, R^{I}, and R^{J} is independently selected from hydrogen and C1-C6 alkyl; and
m is 0, 1, or 2.

In some embodiments, one of X¹, X², X³, X⁴ is N, and each one of the remaining of X¹, X², X³, X⁴ is independently selected from C, C(═O), CH, CR¹ or CR².

In some embodiments, X¹ is N; X² is CR²; X³ is C; and X⁴ is CH.

In some embodiments, X² is N; X¹ is CR¹; X³ is C; and X⁴ is CH.

In some embodiments, X³ is N; X¹ is CR¹; X² is C(═O); and X⁴ is CH.

In some embodiments, X⁴ is N; X¹ is CR¹; X² is CR²; and X³ is C.

In some embodiments, each one of X¹, X², X³, X⁴ is independently selected from C, C(═O), CH, CR¹ or CR².

In some embodiments, ring A is a 5-membered heteroaryl.

In some embodiments, Ring A is

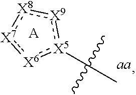

wherein aa represents the point of connection to X³, and each dash bond is independently a single bond or a double, and each one of X⁵, X⁶, X⁷, X⁸ and X⁹ is independently selected from C, (C═O), C═NH, CH, N, O, or S.

In some embodiments, Ring A is selected from the group consisting of thiazolyene, oxazolyene, imidazolyene, pyrazolyene, 1,2,4-triazolyene, 1,2,4-oxadiazolylene and 2-imine-thiazolylene.

In some embodiments, Ring A is selected from the group consisting of

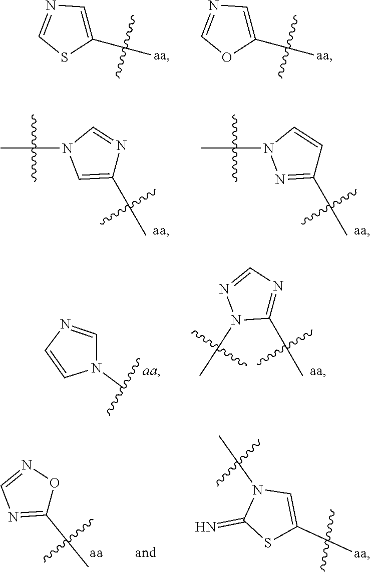

each of which is optionally substituted with one or two R⁵, and aa represents the point of attachment to X³ and the other wave line represents the point of connection to R⁵.

In some embodiments, Ring A is a 6-membered heteroaryl.

In some embodiments, Ring A is

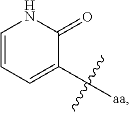

wherein aa represents the point of attachment to X³.

In some embodiments, R¹ is hydrogen. In some embodiments, R¹ is cyano. In some embodiments, R¹ is halo (e.g., Cl or F). In some embodiments, R¹ is 3-10 membered heterocyclyl. In some embodiments, R¹ is 4-6 membered heterocyclyl. In some embodiments, R¹ is 7-10 membered heterocyclyl. In some embodiments, R¹ is

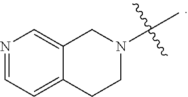 [SMILES: *N1CCc2ccncc2C1]

In some embodiments, R¹ is C1-C6 alkyl optionally substituted with 3-6 membered heterocyclyl optionally substituted with —C(═O)C1-C6 alkyl or —C(═O)OR^{A}. In some embodiments, R¹ is C1-C6 alkyl substituted with 3-6 membered heterocyclyl optionally substituted with —C(═O)C1-C6 alkyl or —C(═O)OR^{A}. In some embodiments, R¹ is C1-C6 alkyl substituted with 3-6 membered heterocyclyl substituted with —C(═O)C1-C6 alkyl or —C(═O)OR^{A}. In some embodiments, R¹ is C1-C6 alkyl substituted with 3-6 membered heterocyclyl. In some embodiments, R¹ is C1-C6 alkyl (i.e., an unsubstituted C1-C6 alkyl).

In some embodiments, the heterocyclyl is piperidinylene, piperidinyl, piperizinylene, or piperizinyl.

In some embodiments, R¹

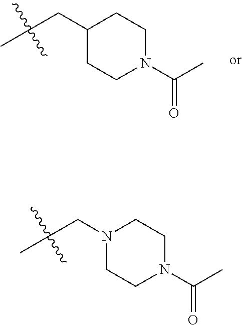 [SMILES: *CC1CCN(C(C)=O)CC1 ; *CN1CCN(C(C)=O)CC1]

wherein the wave line represent the point of attachment to X¹.

In some embodiments, R¹ is —C(═O)-3-6 membered heterocyclyl optionally substituted with C1-C6 alkyl.

In some embodiments, R¹ is

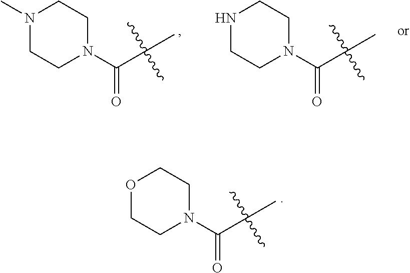

In some embodiments, R¹ is —OR^{B}.

The compound claim 30, wherein R¹ is selected from the group consisting of

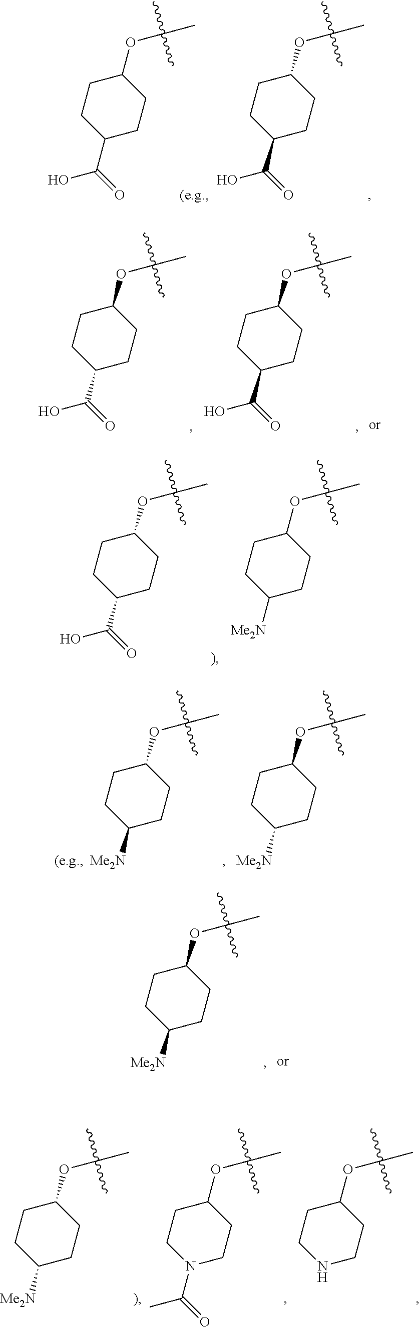

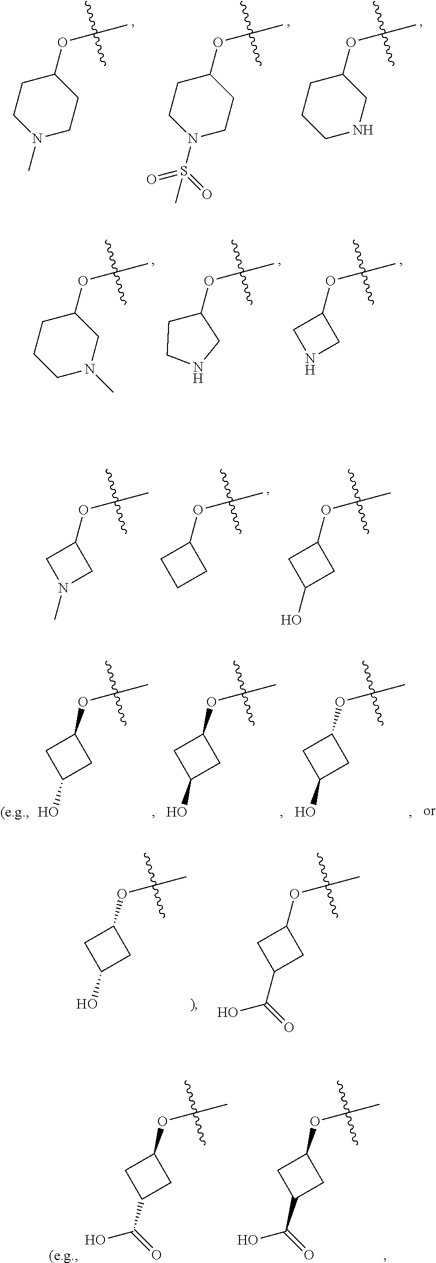

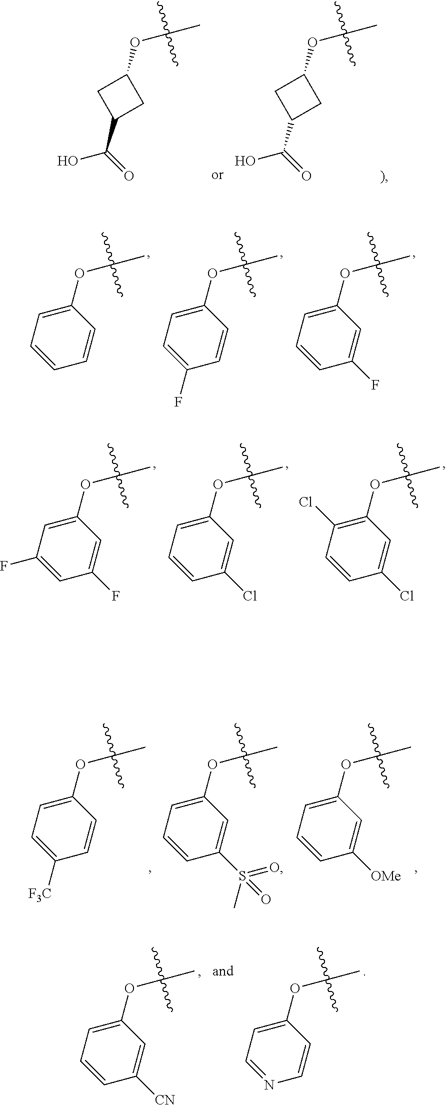

In some embodiments, R² is hydrogen. In some embodiments, R² is halogen (e.g., F, C1). In some embodiments, R² is C1-C6 alkyl (e.g., methyl). In some embodiments, R² is C1-C6 alkoxy (e.g., methoxy, ethoxy).

In some embodiments, R² is —C(═O)-3-6 membered heterocyclyl.

In some embodiments, R² is —NH—C3-C6 cycloalkyl-C(═O)OR^{A}.

In some embodiments, R² is or —O—C3-C6 cycloalkyl-C(═O)OR^{A}.

In some embodiments, R³ is hydrogen. In some embodiments, R³ is halogen (e.g., F or C1).

In some embodiments, R³ is fluoro. In some embodiments, R³ is chloro. In some embodiments, R³ is cyano.

In some embodiments, R³ is C1-C6 alkyl (e.g., methyl, ethyl).

In some embodiments, R³ is halogen or C1-C6 alkyl.

In some embodiments, R³ is fluoro, chloro, or methyl.

In some embodiments, R³ is C3-C6 cycloalkyl (e.g., cyclopropyl).

In some embodiments, R³ is —X—R^{G}, wherein X is —NH—, —NHC(═O)O—, —O—, or CH₂.

In some embodiments, R³ is selected from the list of structures consisting of

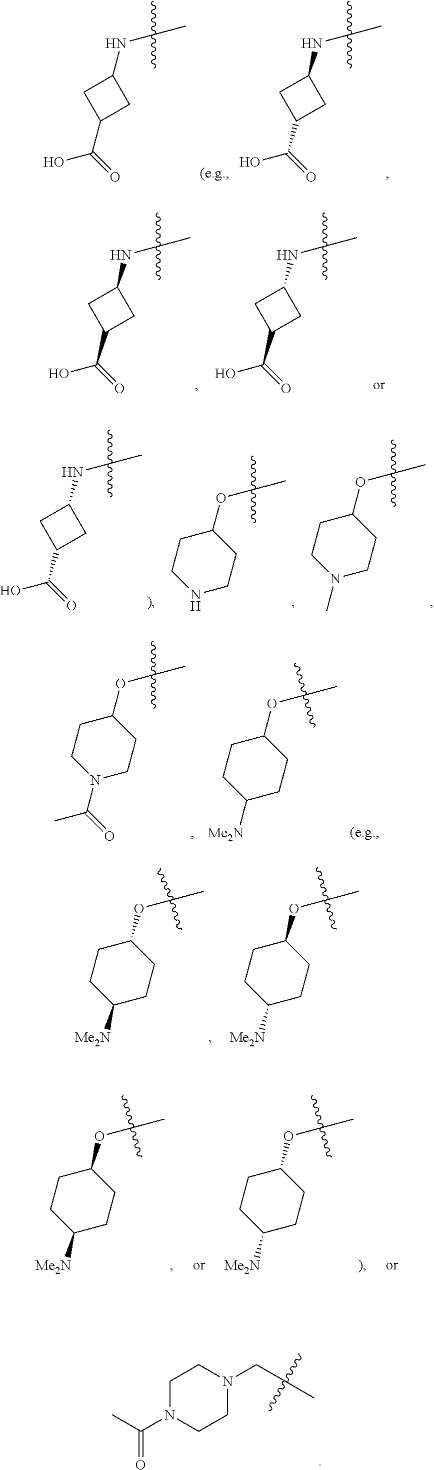

In some embodiments, R³ is

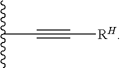

In some embodiments, R³ is selected from the group consisting of

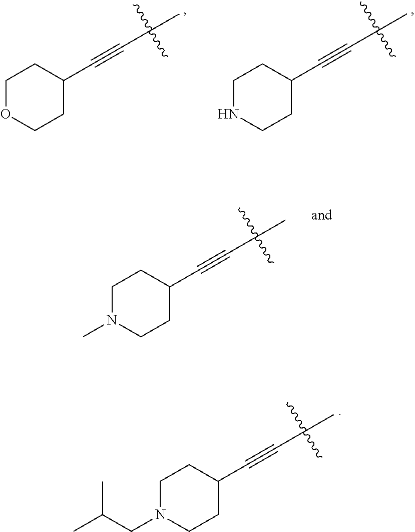

In some embodiments, R⁴ is hydrogen. In some embodiments, R⁴ is C1-C6 alkyl (e.g., methyl, ethyl).

In some embodiments, m is 0. In some embodiments, m is 1 or 2. In some embodiments, m is 1.

In some embodiments, R⁵ is hydrogen.

In some embodiments, R⁵ is C1-C6 alkyl optionally substituted with 3-6 membered heterocyclyl. In some embodiments, R⁵ is C1-C6 alkyl substituted with 3-6 membered heterocyclyl. In some embodiments, R⁵ is C1-C6 alky (e.g., methyl, ethyl).

In some embodiments, the heterocyclyl group is morpholinyl

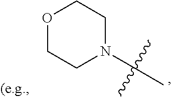 [SMILES: *N1CCOCC1]

wherein the wave line represents the point of connection to the C1-C6 alkyl group).

In some embodiments, R⁵ is

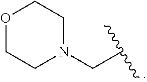 [SMILES: *CN1CCOCC1]

In some embodiments, R⁵ is —X—R^{E}.

In some embodiments, R⁵ is —(NH)(C═O)R^{E}, —(NH)(C═O)O—R^{E}, —NH—R^{E}, or —(C═O)R^{E}—.

In some embodiments, R⁵ is —(NH)(C═O)R^{E}. In some embodiments, R⁵ is —(NH)(C═O)O—R^{E}. In some embodiments, R⁵ is —NH—R^{E}. In some embodiments, R⁵ is —(C═O)R^{E}—.

In some embodiments, R⁵ is selected from the group consisting of

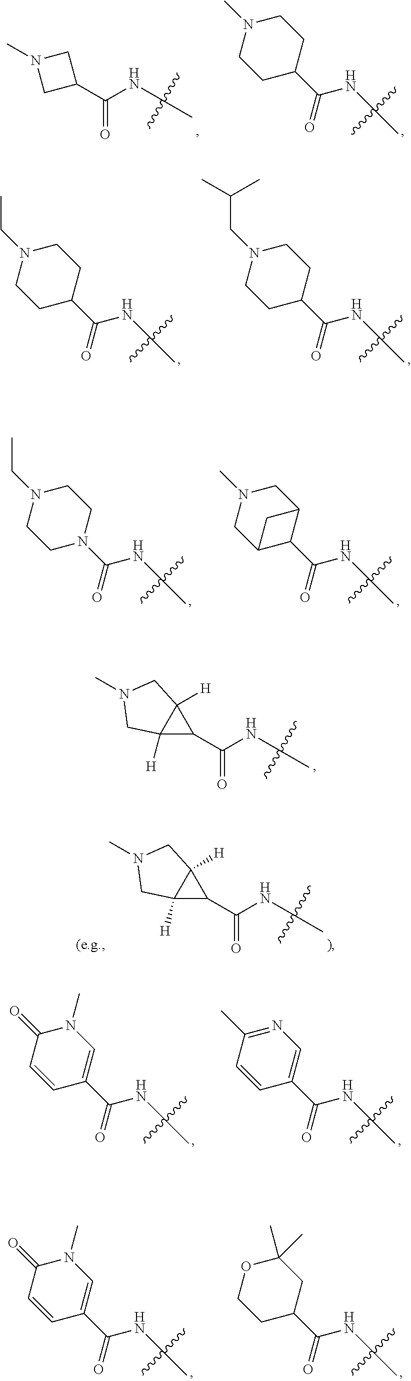

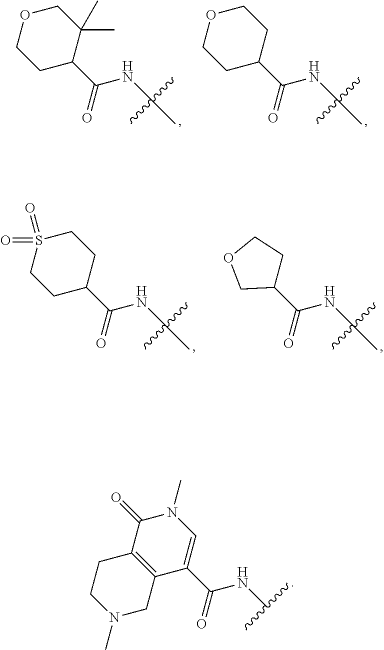 [SMILES: *NC(=O)C1CCOCC1(C)C ; *NC(=O)C1CCOCC1 ; *NC(=O)C1CCS(=O)(=O)CC1 ; *NC(=O)C1CCOC1 ; *NC(=O)c1cn(C)c(=O)c2c1CN(C)CC2]

In some embodiments, R⁵ is

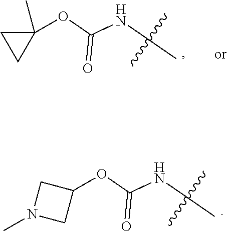

In some embodiments, R⁵ is

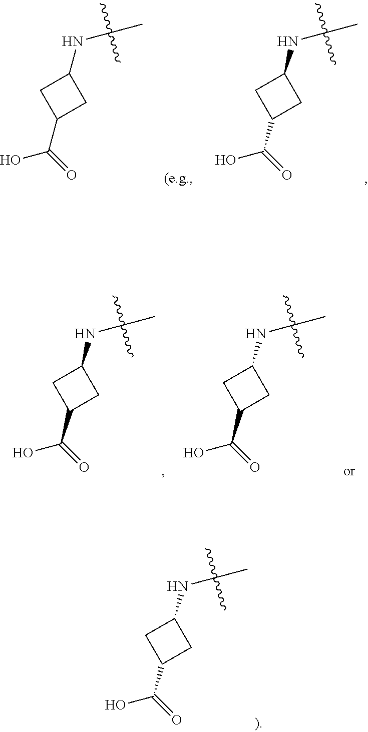

In some embodiments, R⁵ is

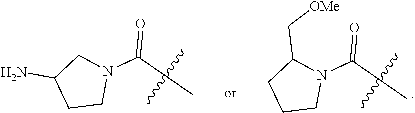 [SMILES: *C(=O)N1CCC(N)C1 ; *C(=O)N1CCCC1COC]

In some embodiments, R⁵ is —C3-C6 cycloalkyl-C(═O)OR^{A}.

In some embodiments, R⁵ is

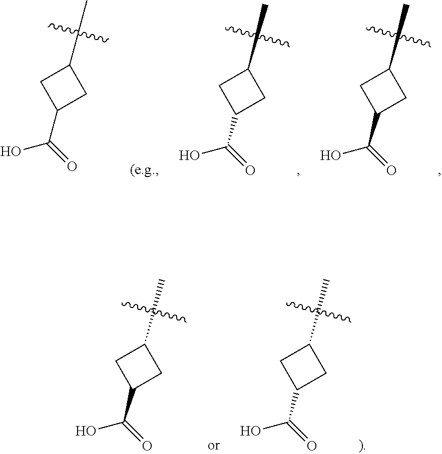

In some embodiments, R⁵ is

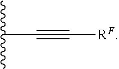

In some embodiments, R^{F} is a 3-6 membered heterocyclyl optionally substituted with C2-C6 alkynyl. In some embodiments, R^{F} is a 3-6 membered heterocyclyl substituted with C2-C6 alkynyl.

In some embodiments, R⁵ is selected from the group consisting of

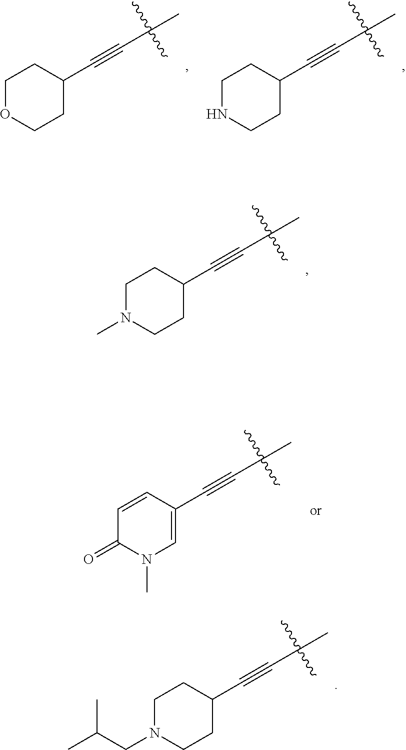

In some embodiments, R^{F} is C2-C6 alkynyl optionally substituted with hydroxy. In some embodiments, R^{F} is C2-C6 alkynyl substituted with hydroxy.

In some embodiments, R⁵ is

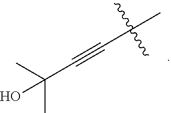 [SMILES: *C#CC(C)(C)O]

In some embodiments, R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a (i) C6-C10 aryl optionally substituted with a 3-10 membered heterocyclyl optionally substituted with 1-2 independently selected C1-C6 alkyl or —C(═O)OR′; (ii) a 3-6 membered heterocyclyl; or a (iii) a 5-6 membered heteroaryl optionally substituted with 1-2 substituents independently selected from C1-C6 alkyl, C1-C6 haloalkyl, C1-C6 alkoxy, 3-10 membered heterocyclyl optionally substituted with 1-2 independently selected C1-C6 alkyl or C(═O)OR′, and 5-6 membered heteroaryl optionally substituted with C1-C6 alkyl;

In some embodiments, R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a C6-C10 aryl (e.g., phenyl) optionally substituted with a 3-10 membered heterocyclyl optionally substituted with 1-2 independently selected C1-C6 alkyl or —C(═O)OR′.

In some embodiments, R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a phenyl ring

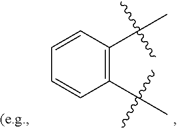 [SMILES: *c1ccccc1*]

wherein the wavy lines represent the point of attachment to Ring A), which is further optionally substituted with optionally substituted with a 3-10 membered heterocyclyl optionally substituted with 1-2 independently selected C1-C6 alkyl or —C(═O)OR′.

In some embodiments, R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a structure selected from a group consisting of

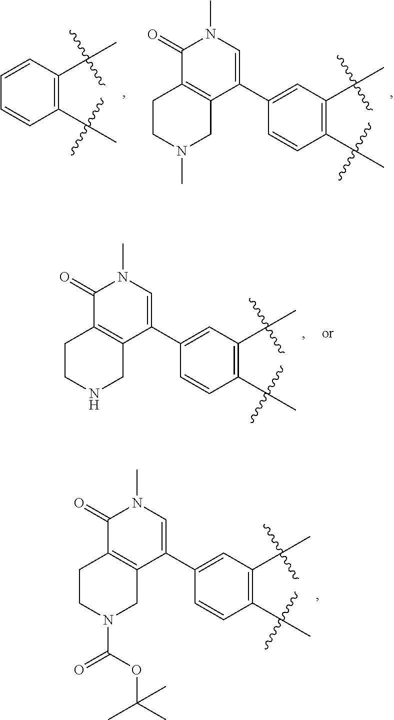 [SMILES: *c1ccccc1* ; *c1ccc(-c2cn(C)c(=O)c3c2CN(C)CC3)cc1* ; *c1ccc(-c2cn(C)c(=O)c3c2CNCC3)cc1* ; *c1ccc(-c2cn(C)c(=O)c3c2CN(C(=O)OC(C)(C)C)CC3)cc1*]

wherein the wavy lines represent the points of attachment to Ring A.

In some embodiments, R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a 3-6 membered heterocyclyl.

In some embodiments, R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a pyrrolidine ring

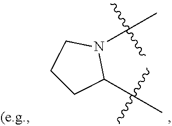 [SMILES: *C1CCCN1*]

wherein the wavy lines represent the points of attachment to Ring A).

In some embodiments, R⁵ and the carbon and/or nitrogen atom to which it is attached, forms a bond with an adjacent carbon or nitrogen atom, replacing the hydrogen atom on the adjacent carbon or nitrogen atom, and R⁵ and the two adjacent carbon and/or nitrogen atoms in Ring A from a 5-6 membered heteroaryl optionally substituted with 1-2 substituents independently selected from C1-C6 alkyl, C1-C6 haloalkyl, C1-C6 alkoxy, 3-10 membered heterocyclyl optionally substituted with 1-2 independently selected C1-C6 alkyl or C(═O)OR′, and 5-6 membered heteroaryl optionally substituted with C1-C6 alkyl.

In some embodiments, R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A from a ring structure selected from the list of structures consisting of

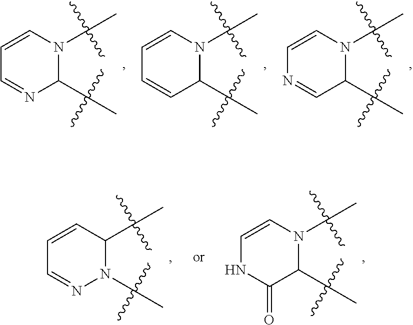 [SMILES: *C1N=CC=CN1* ; *C1C=CC=CN1* ; *C1C=NC=CN1* ; *C1C=CC=NN1* ; *C1C(=O)NC=CN1*]

each of which is optionally substituted with 1-2 substituents independently selected from C1-C6 alkyl, C1-C6 haloalkyl, C1-C6 alkoxy, 3-10 membered heterocyclyl optionally substituted with 1-2 independently selected C1-C6 alkyl or C(═O)OR′, and 5-6 membered heteroaryl optionally substituted with C1-C6 alkyl, and the wavy lines represent the points of attachment to Ring A.

In some embodiments, R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A from a ring structure selected from the list of structures consisting of

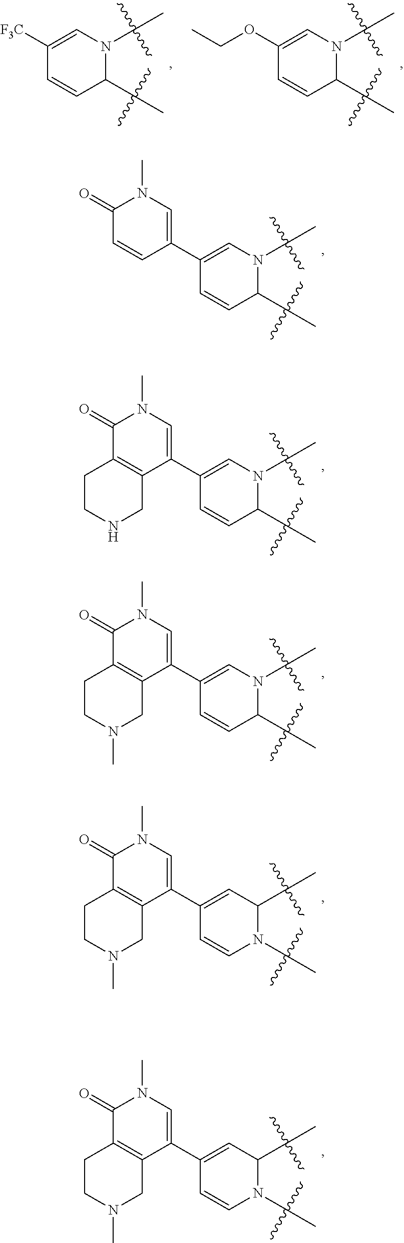

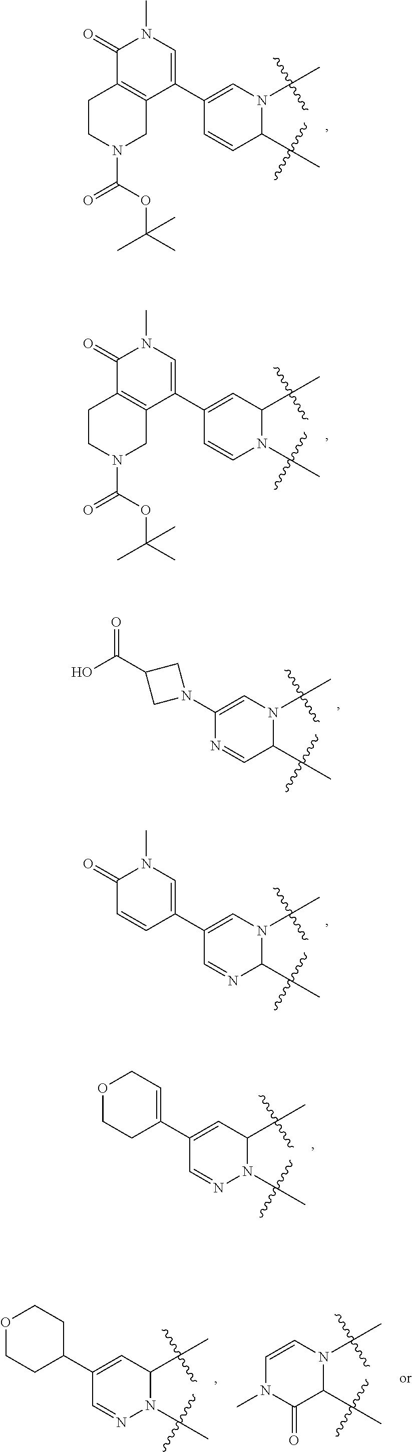

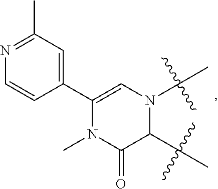 [SMILES: *C1C(=O)N(C)C(c2ccnc(C)c2)=CN1*]

wherein the wavy lines represent the points of attachment to Ring A.

In some embodiments, R^{E} is a 3-10 membered heterocyclyl optionally substituted with 1-4 independently selected C1-C6 alkyl optionally substituted with C1-C6 alkoxy, NR^{I}R^{J}, or —C(═O)OH. In some embodiments, R^{E} is a 3-10 membered heterocyclyl optionally substituted with 1-4 independently selected C1-C6 alkyl. In some embodiments, R^{E} is a pyridone optionally substituted with 1-4 independently selected C1-C6 alkyl. In some embodiments, R^{E} is a tetrahydropyran optionally substituted with 1-4 independently selected C1-C6 alkyl. In some embodiments, R^{E} is a tetrahydro-2H-thiopyran 1,1-dioxide optionally substituted with 1-4 independently selected C1-C6 alkyl.

In some embodiments, R^{E} is a 5-6 membered heteroaryl optionally substituted with 1-4 independently selected C1-C6 alkyl optionally substituted with C1-C6 alkoxy, NR^{I}R^{J}, or —C(═O)OH.

In some embodiments, R^{E} is a C3-C6 cycloalkyl optionally substituted with 1-4 independently selected C1-C6 alkyl optionally substituted with C1-C6 alkoxy, NR^{I}R^{J}, or —C(═O)OH.

In some embodiments, the compound is a compound of formula (I-1).

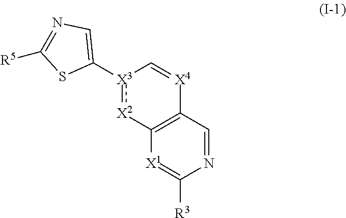

In some embodiments, the compound is a compound of formula (I-2).

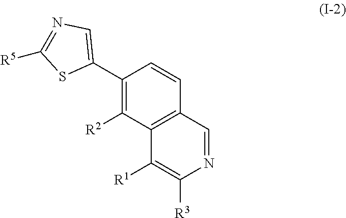

In some embodiments, the compound is a compound of formula (I-3).

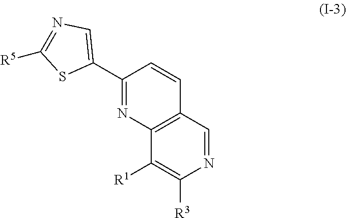

In some embodiments, the compound is a compound of formula (I-4).

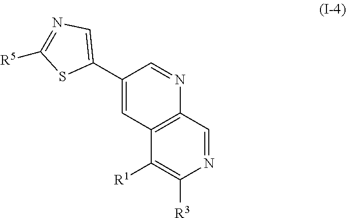

In some embodiments, the compound is a compound of formula (I-5).

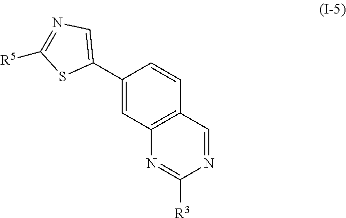

In some embodiments, the compound is a compound of formula (I-6).

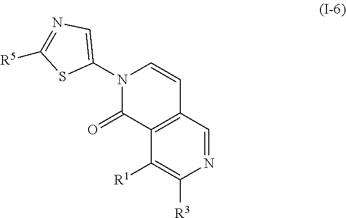

In some embodiments, the compound is a compound of formula (I-7).

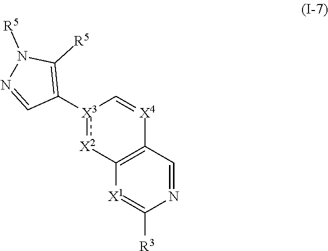

In some embodiments, the compound is a compound of formula (I-8).

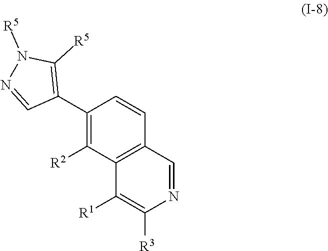

In some embodiments, the compound is a compound of formula (I-9).

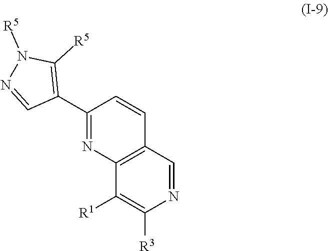

In some embodiments, the compound is a compound of formula (I-10).

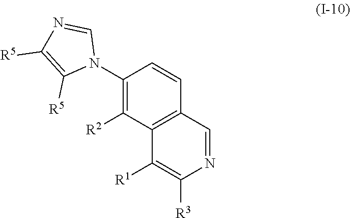

In some embodiments, the compound is a compound of formula (I-11).

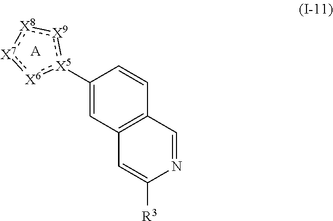

In some embodiments, the compound is a compound of formula (I-12).

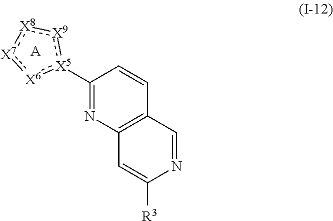

In some embodiments, the compound is a compound of formula (I-13).

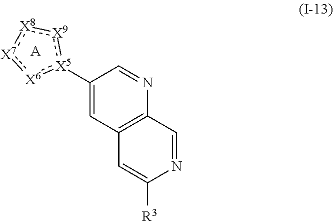

In some embodiments, the compound is a compound of formula (I-14).

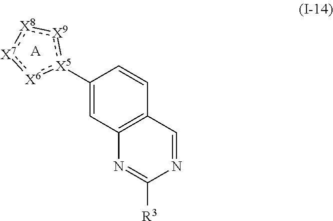

In some embodiments, the compound is a compound of formula (I-15).

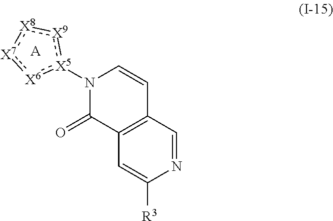

In some embodiments, the compound is a compound of formula (I-16).

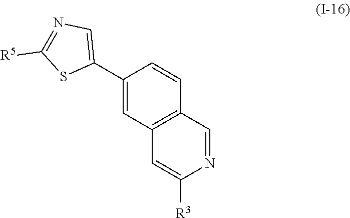

In some embodiments, the compound is a compound of formula (I-17).

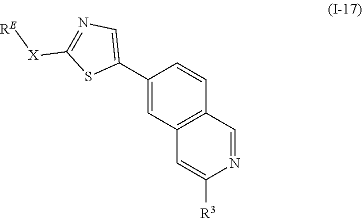

In some embodiments, the compound is a compound of formula (I-18).

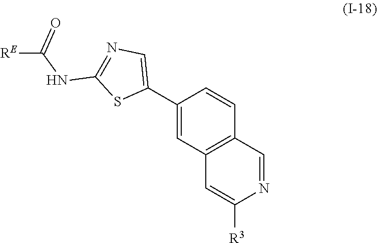

In some embodiments, the compound is a compound of formula (I-19).

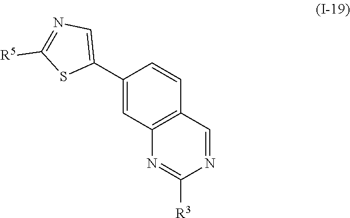

In some embodiments, the compound is a compound of formula (I-20).

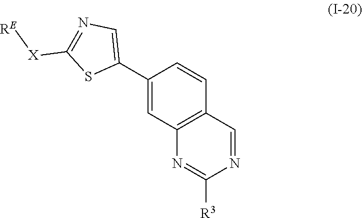

In some embodiments, the compound is a compound of formula (I-21).

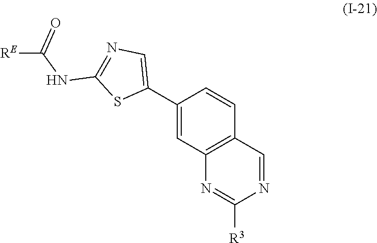

In some embodiments, the compound of Formula (I) is selected from a compound in Table 1, or a pharmaceutically acceptable salt thereof.

TABLE 1
Selected Compounds of Formula (I)

| Compound | |
|---|---|
| No. | Structure |
| 1 | 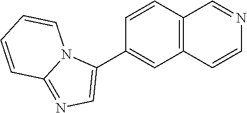 [SMILES: c1ccn2c(-c3ccc4cnccc4c3)cnc2c1] |
| 2 | 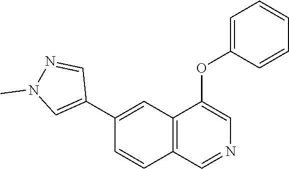 [SMILES: Cn1cc(-c2ccc3cncc(Oc4ccccc4)c3c2)cn1] |
| 3 | 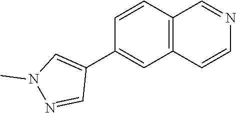 [SMILES: Cn1cc(-c2ccc3cnccc3c2)cn1] |
| 4 | 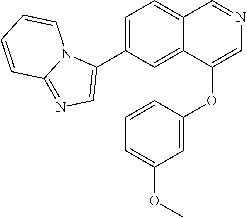 [SMILES: COc1cccc(Oc2cncc3ccc(-c4cnc5ccccn45)cc23)c1] |
| 5 | 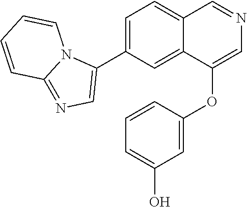 [SMILES: Oc1cccc(Oc2cncc3ccc(-c4cnc5ccccn45)cc23)c1] |
| 6 | 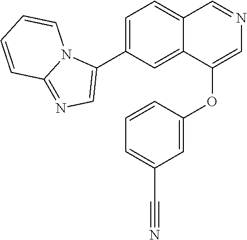 [SMILES: N#Cc1cccc(Oc2cncc3ccc(-c4cnc5ccccn45)cc23)c1] |
| 7 | 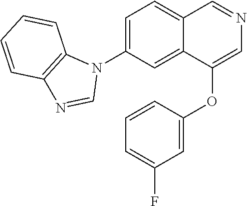 [SMILES: Fc1cccc(Oc2cncc3ccc(-n4cnc5ccccc54)cc23)c1] |
| 8 | 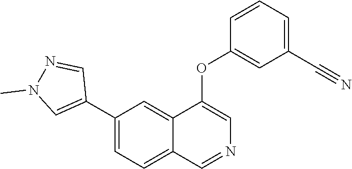 [SMILES: Cn1cc(-c2ccc3cncc(Oc4cccc(C#N)c4)c3c2)cn1] |
| 9 | 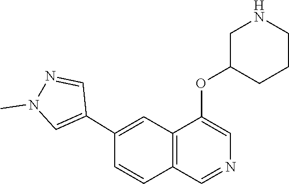 [SMILES: Cn1cc(-c2ccc3cncc(OC4CCCNC4)c3c2)cn1] |
| 10 | 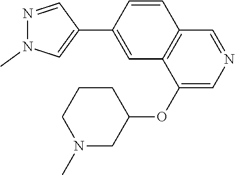 [SMILES: CN1CCCC(Oc2cncc3ccc(-c4cnn(C)c4)cc23)C1] |
| 11 | 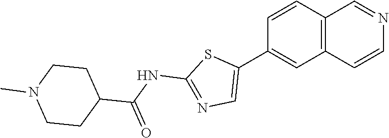 [SMILES: CN1CCC(C(=O)Nc2ncc(-c3ccc4cnccc4c3)s2)CC1] |
| 12 | 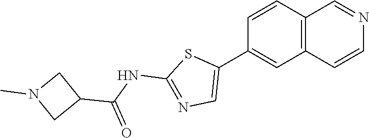 [SMILES: CN1CC(C(=O)Nc2ncc(-c3ccc4cnccc4c3)s2)C1] |
| 13 | 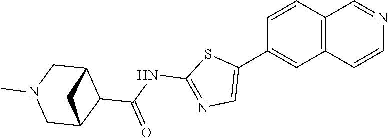 [SMILES: CN1C[C@H]2C[C@@H](C1)C2C(=O)Nc1ncc(-c2ccc3cnccc3c2)s1] |
| 14 | 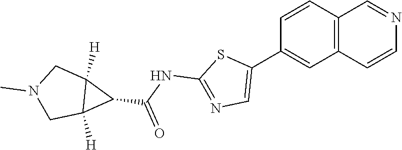 [SMILES: CN1C[C@@H]2[C@H](C1)[C@H]2C(=O)Nc1ncc(-c2ccc3cnccc3c2)s1] |
| 15 | 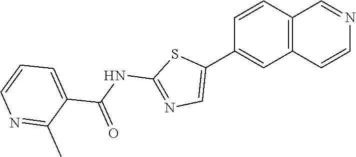 [SMILES: Cc1ncccc1C(=O)Nc1ncc(-c2ccc3cnccc3c2)s1] |
| 16 | 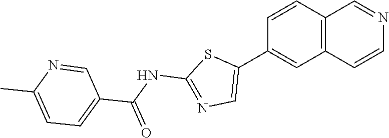 [SMILES: Cc1ccc(C(=O)Nc2ncc(-c3ccc4cnccc4c3)s2)cn1] |
| 17 | 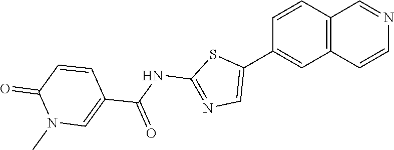 [SMILES: Cn1cc(C(=O)Nc2ncc(-c3ccc4cnccc4c3)s2)ccc1=O] |
| 18 | 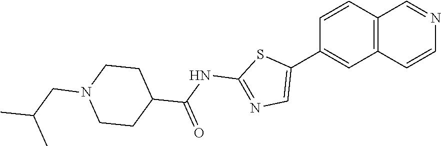 [SMILES: CC(C)CN1CCC(C(=O)Nc2ncc(-c3ccc4cnccc4c3)s2)CC1] |
| 19 | 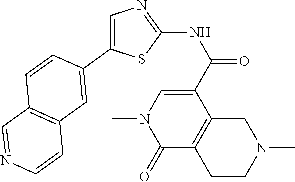 [SMILES: CN1CCc2c(c(C(=O)Nc3ncc(-c4ccc5cnccc5c4)s3)cn(C)c2=O)C1] |
| 20 | 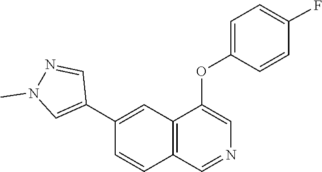 [SMILES: Cn1cc(-c2ccc3cncc(Oc4ccc(F)cc4)c3c2)cn1] |
| 21 | 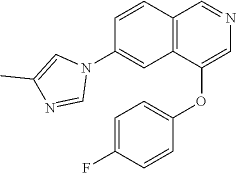 [SMILES: Cc1cn(-c2ccc3cncc(Oc4ccc(F)cc4)c3c2)cn1] |
| 22 | 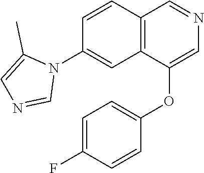 [SMILES: Cc1cncn1-c1ccc2cncc(Oc3ccc(F)cc3)c2c1] |
| 23 | 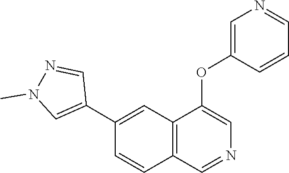 [SMILES: Cn1cc(-c2ccc3cncc(Oc4cccnc4)c3c2)cn1] |
| 24 | 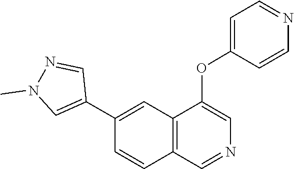 [SMILES: Cn1cc(-c2ccc3cncc(Oc4ccncc4)c3c2)cn1] |
| 25 | 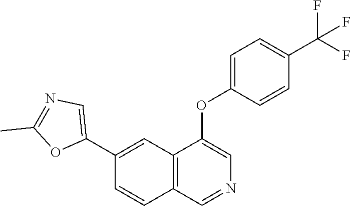 [SMILES: Cc1ncc(-c2ccc3cncc(Oc4ccc(C(F)(F)F)cc4)c3c2)o1] |
| 26 | 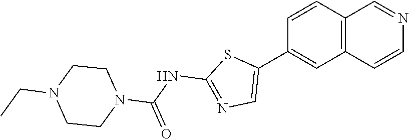 [SMILES: CCN1CCN(C(=O)Nc2ncc(-c3ccc4cnccc4c3)s2)CC1] |
| 27 | 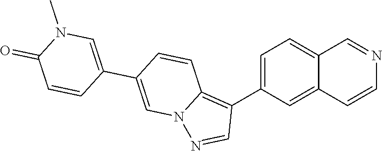 [SMILES: Cn1cc(-c2ccc3c(-c4ccc5cnccc5c4)cnn3c2)ccc1=O] |
| 28 | 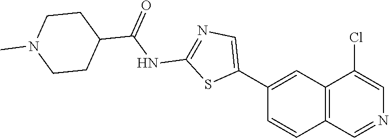 [SMILES: CN1CCC(C(=O)Nc2ncc(-c3ccc4cncc(Cl)c4c3)s2)CC1] |
| 29 | 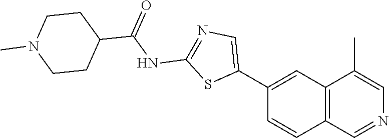 [SMILES: Cc1cncc2ccc(-c3cnc(NC(=O)C4CCN(C)CC4)s3)cc12] |
| 30 | 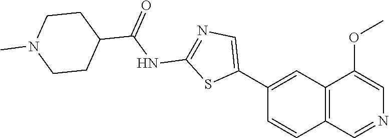 [SMILES: COc1cncc2ccc(-c3cnc(NC(=O)C4CCN(C)CC4)s3)cc12] |
| 31 | 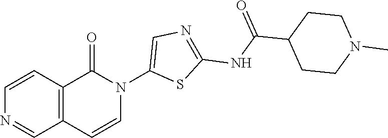 [SMILES: CN1CCC(C(=O)Nc2ncc(-n3ccc4cnccc4c3=O)s2)CC1] |
| 32 | 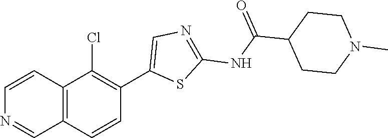 [SMILES: CN1CCC(C(=O)Nc2ncc(-c3ccc4cnccc4c3Cl)s2)CC1] |
| 33 | 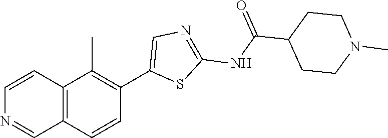 [SMILES: Cc1c(-c2cnc(NC(=O)C3CCN(C)CC3)s2)ccc2cnccc12] |
| 34 | 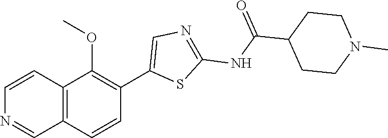 [SMILES: COc1c(-c2cnc(NC(=O)C3CCN(C)CC3)s2)ccc2cnccc12] |
| 35 | 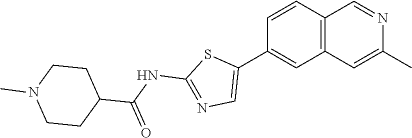 |
| 36 | 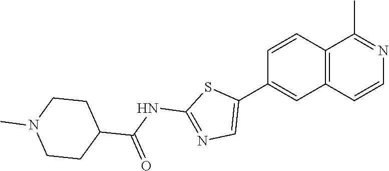 [SMILES: Cc1nccc2cc(-c3cnc(NC(=O)C4CCN(C)CC4)s3)ccc12] |
| 37 | 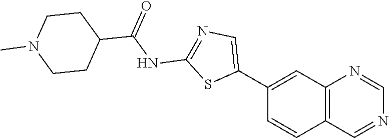 [SMILES: CN1CCC(C(=O)Nc2ncc(-c3ccc4cncnc4c3)s2)CC1] |
| 38 | 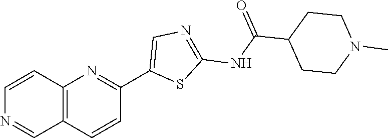 [SMILES: CN1CCC(C(=O)Nc2ncc(-c3ccc4cnccc4n3)s2)CC1] |
| 39 | 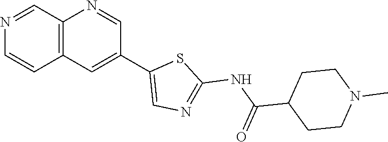 [SMILES: CN1CCC(C(=O)Nc2ncc(-c3cnc4cnccc4c3)s2)CC1] |
| 40 | 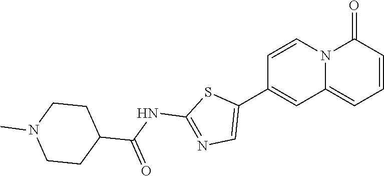 [SMILES: CN1CCC(C(=O)Nc2ncc(-c3ccn4c(=O)cccc4c3)s2)CC1] |
| 41 | 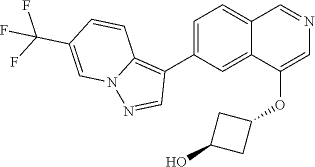 [SMILES: O[C@H]1C[C@H](Oc2cncc3ccc(-c4cnn5cc(C(F)(F)F)ccc45)cc23)C1] |
| 42 | 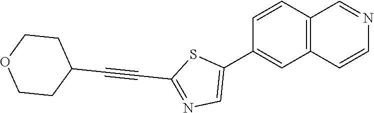 [SMILES: C(#CC1CCOCC1)c1ncc(-c2ccc3cnccc3c2)s1] |
| 43 | 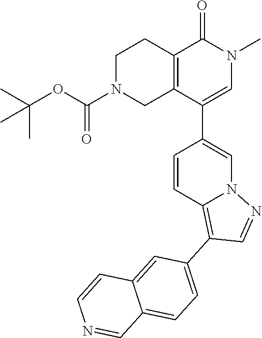 [SMILES: Cn1cc(-c2ccc3c(-c4ccc5cnccc5c4)cnn3c2)c2c(c1=O)CCN(C(=O)OC(C)(C)C)C2] |
| 44 | 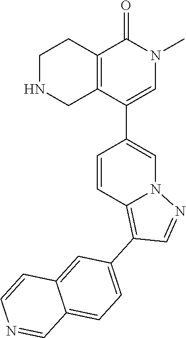 [SMILES: Cn1cc(-c2ccc3c(-c4ccc5cnccc5c4)cnn3c2)c2c(c1=O)CCNC2] |
| 45 | 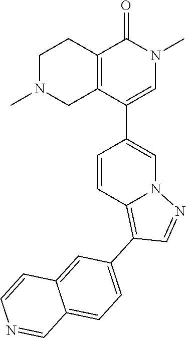 [SMILES: CN1CCc2c(c(-c3ccc4c(-c5ccc6cnccc6c5)cnn4c3)cn(C)c2=O)C1] |
| 46 | 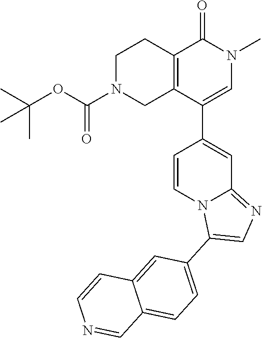 [SMILES: Cn1cc(-c2ccn3c(-c4ccc5cnccc5c4)cnc3c2)c2c(c1=O)CCN(C(=O)OC(C)(C)C)C2] |
| 47 | 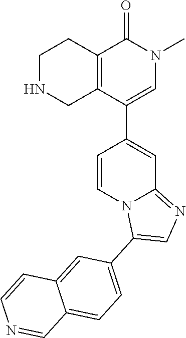 [SMILES: Cn1cc(-c2ccn3c(-c4ccc5cnccc5c4)cnc3c2)c2c(c1=O)CCNC2] |
| 48 | 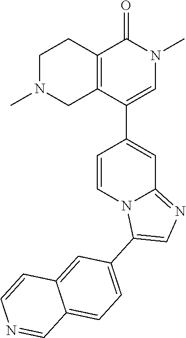 [SMILES: CN1CCc2c(c(-c3ccn4c(-c5ccc6cnccc6c5)cnc4c3)cn(C)c2=O)C1] |
| 49 | 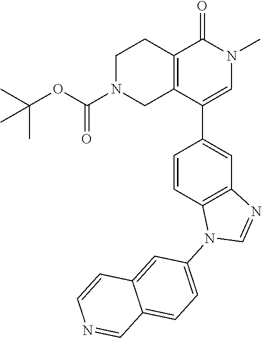 [SMILES: Cn1cc(-c2ccc3c(c2)ncn3-c2ccc3cnccc3c2)c2c(c1=O)CCN(C(=O)OC(C)(C)C)C2] |
| 50 | 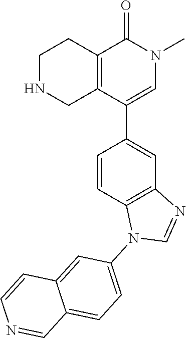 [SMILES: Cn1cc(-c2ccc3c(c2)ncn3-c2ccc3cnccc3c2)c2c(c1=O)CCNC2] |
| 51 | 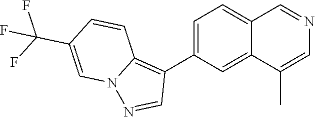 [SMILES: Cc1cncc2ccc(-c3cnn4cc(C(F)(F)F)ccc34)cc12] |
| 52 | 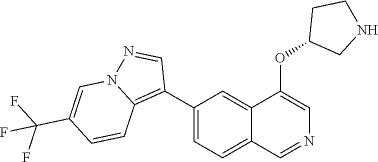 [SMILES: FC(F)(F)c1ccc2c(-c3ccc4cncc(O[C@@H]5CCNC5)c4c3)cnn2c1] |
| 53 | 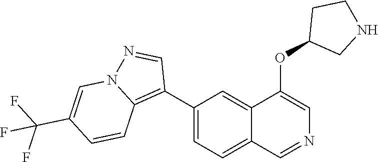 [SMILES: FC(F)(F)c1ccc2c(-c3ccc4cncc(O[C@H]5CCNC5)c4c3)cnn2c1] |
| 54 | 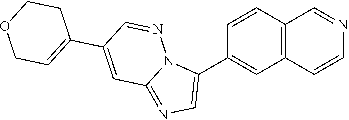 [SMILES: C1=C(c2cnn3c(-c4ccc5cnccc5c4)cnc3c2)CCOC1] |
| 55 | 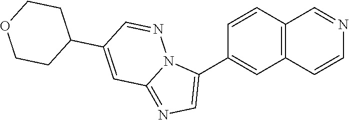 [SMILES: c1cc2cc(-c3cnc4cc(C5CCOCC5)cnn34)ccc2cn1] |
| 56 | 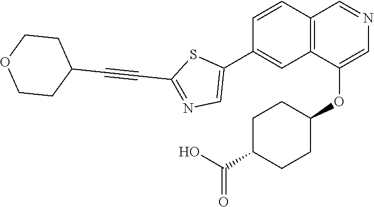 [SMILES: O=C(O)[C@H]1CC[C@H](Oc2cncc3ccc(-c4cnc(C#CC5CCOCC5)s4)cc23)CC1] |
| 57 | 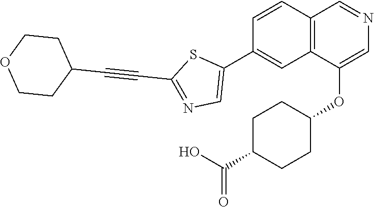 [SMILES: O=C(O)[C@H]1CC[C@@H](Oc2cncc3ccc(-c4cnc(C#CC5CCOCC5)s4)cc23)CC1] |
| 58 | 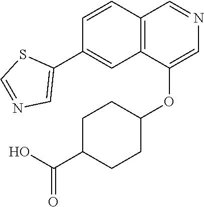 [SMILES: O=C(O)C1CCC(Oc2cncc3ccc(-c4cncs4)cc23)CC1] |
| 59 | 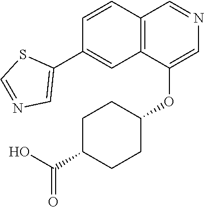 [SMILES: O=C(O)[C@H]1CC[C@@H](Oc2cncc3ccc(-c4cncs4)cc23)CC1] |
| 60 | 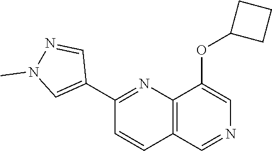 [SMILES: Cn1cc(-c2ccc3cncc(OC4CCC4)c3n2)cn1] |
| 61 | 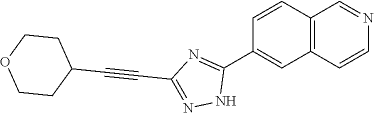 [SMILES: C(#CC1CCOCC1)c1n[nH]c(-c2ccc3cnccc3c2)n1] |
| 62 | 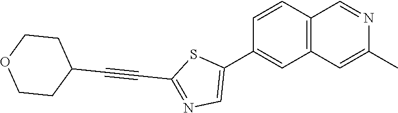 [SMILES: Cc1cc2cc(-c3cnc(C#CC4CCOCC4)s3)ccc2cn1] |
| 63 | 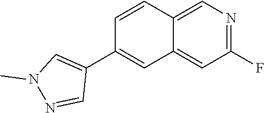 [SMILES: Cn1cc(-c2ccc3cnc(F)cc3c2)cn1] |
| 64 | 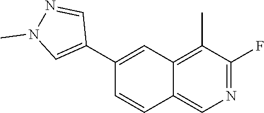 [SMILES: Cc1c(F)ncc2ccc(-c3cnn(C)c3)cc12] |
| 65 | 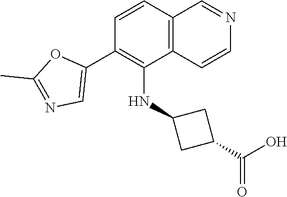 [SMILES: Cc1ncc(-c2ccc3cnccc3c2N[C@H]2C[C@H](C(=O)O)C2)o1] |
| 66 | 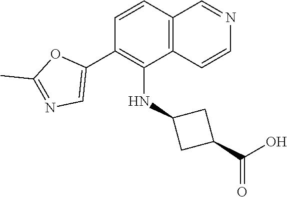 [SMILES: Cc1ncc(-c2ccc3cnccc3c2N[C@H]2C[C@@H](C(=O)O)C2)o1] |
| 67 | 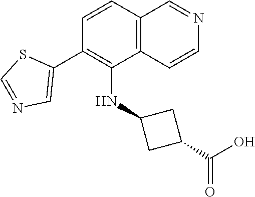 [SMILES: O=C(O)[C@H]1C[C@H](Nc2c(-c3cncs3)ccc3cnccc23)C1] |
| 68 | 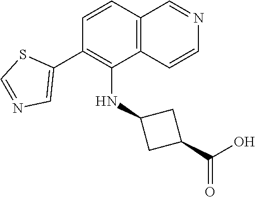 [SMILES: O=C(O)[C@H]1C[C@@H](Nc2c(-c3cncs3)ccc3cnccc23)C1] |
| 69 | 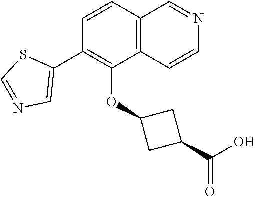 [SMILES: O=C(O)[C@H]1C[C@@H](Oc2c(-c3cncs3)ccc3cnccc23)C1] |
| 70 | 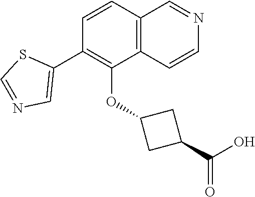 [SMILES: O=C(O)[C@H]1C[C@H](Oc2c(-c3cncs3)ccc3cnccc23)C1] |
| 71 | 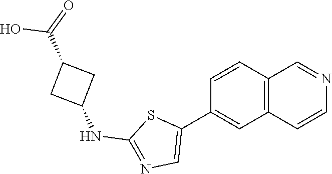 [SMILES: O=C(O)[C@H]1C[C@@H](Nc2ncc(-c3ccc4cnccc4c3)s2)C1] |
| 72 | 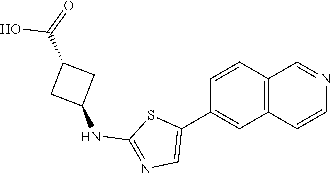 [SMILES: O=C(O)[C@H]1C[C@H](Nc2ncc(-c3ccc4cnccc4c3)s2)C1] |
| 73 | 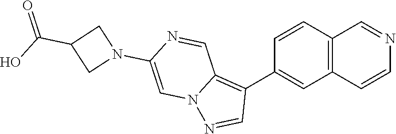 [SMILES: O=C(O)C1CN(c2cn3ncc(-c4ccc5cnccc5c4)c3cn2)C1] |
| 74 | 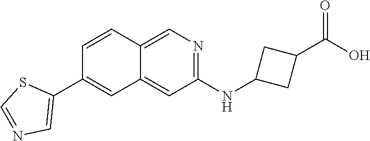 [SMILES: O=C(O)C1CC(Nc2cc3cc(-c4cncs4)ccc3cn2)C1] |
| 75 | 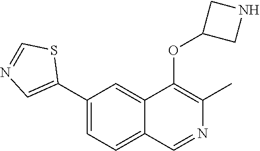 [SMILES: Cc1ncc2ccc(-c3cncs3)cc2c1OC1CNC1] |
| 76 | 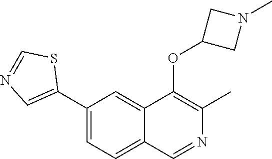 [SMILES: Cc1ncc2ccc(-c3cncs3)cc2c1OC1CN(C)C1] |
| 77 | 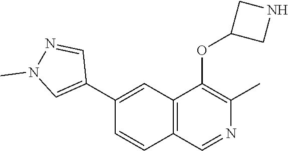 [SMILES: Cc1ncc2ccc(-c3cnn(C)c3)cc2c1OC1CNC1] |
| 78 | 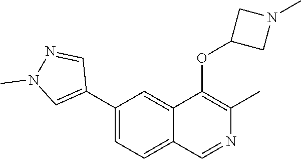 |
| 79 | 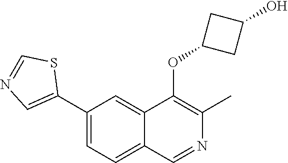 [SMILES: Cc1ncc2ccc(-c3cncs3)cc2c1O[C@H]1C[C@@H](O)C1] |
| 80 | 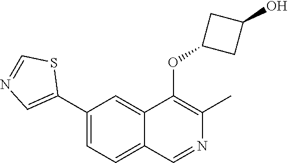 [SMILES: Cc1ncc2ccc(-c3cncs3)cc2c1O[C@H]1C[C@H](O)C1] |
| 81 | 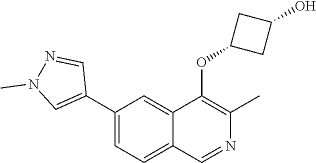 [SMILES: Cc1ncc2ccc(-c3cnn(C)c3)cc2c1O[C@H]1C[C@@H](O)C1] |
| 82 | 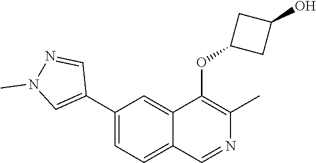 [SMILES: Cc1ncc2ccc(-c3cnn(C)c3)cc2c1O[C@H]1C[C@H](O)C1] |
| 83 | 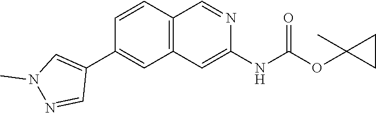 [SMILES: Cn1cc(-c2ccc3cnc(NC(=O)OC4(C)CC4)cc3c2)cn1] |
| 84 | 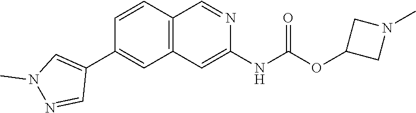 |
| 85 | 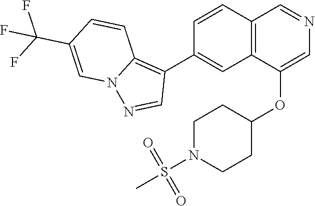 [SMILES: CS(=O)(=O)N1CCC(Oc2cncc3ccc(-c4cnn5cc(C(F)(F)F)ccc45)cc23)CC1] |
| 86 | 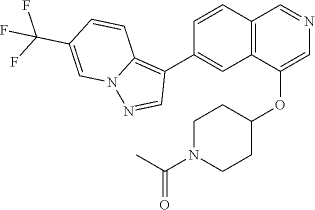 [SMILES: CC(=O)N1CCC(Oc2cncc3ccc(-c4cnn5cc(C(F)(F)F)ccc45)cc23)CC1] |
| 87 | 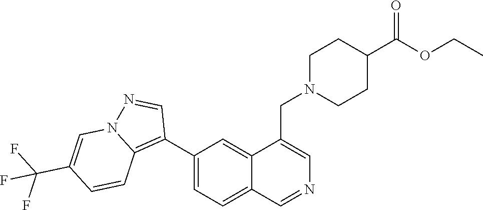 [SMILES: CCOC(=O)C1CCN(Cc2cncc3ccc(-c4cnn5cc(C(F)(F)F)ccc45)cc23)CC1] |
| 88 | 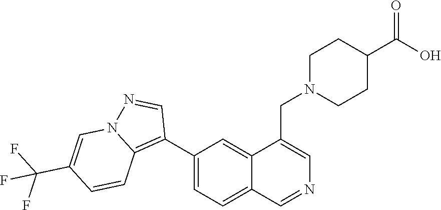 [SMILES: O=C(O)C1CCN(Cc2cncc3ccc(-c4cnn5cc(C(F)(F)F)ccc45)cc23)CC1] |
| 89 | 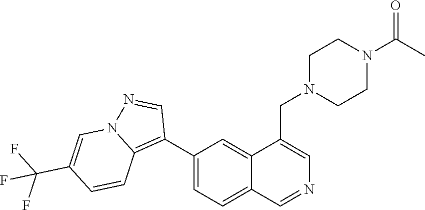 [SMILES: CC(=O)N1CCN(Cc2cncc3ccc(-c4cnn5cc(C(F)(F)F)ccc45)cc23)CC1] |
| 90 | 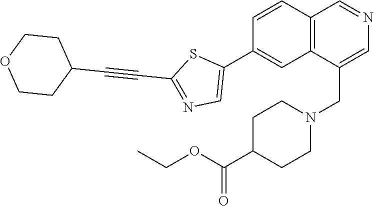 [SMILES: CCOC(=O)C1CCN(Cc2cncc3ccc(-c4cnc(C#CC5CCOCC5)s4)cc23)CC1] |
| 91 | 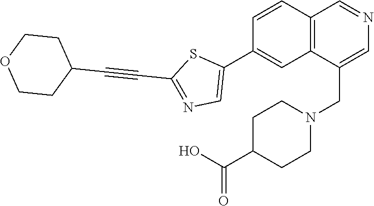 [SMILES: O=C(O)C1CCN(Cc2cncc3ccc(-c4cnc(C#CC5CCOCC5)s4)cc23)CC1] |
| 92 | 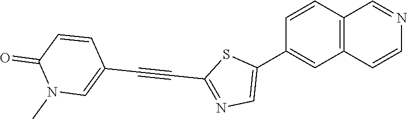 [SMILES: Cn1cc(C#Cc2ncc(-c3ccc4cnccc4c3)s2)ccc1=O] |
| 93 | 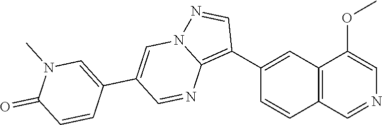 [SMILES: COc1cncc2ccc(-c3cnn4cc(-c5ccc(=O)n(C)c5)cnc34)cc12] |
| 94 | 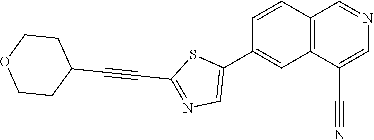 [SMILES: N#Cc1cncc2ccc(-c3cnc(C#CC4CCOCC4)s3)cc12] |
| 95 | 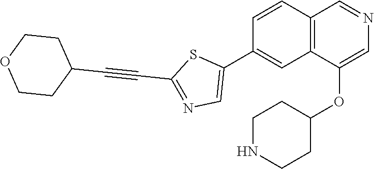 [SMILES: C(#CC1CCOCC1)c1ncc(-c2ccc3cncc(OC4CCNCC4)c3c2)s1] |
| 96 | 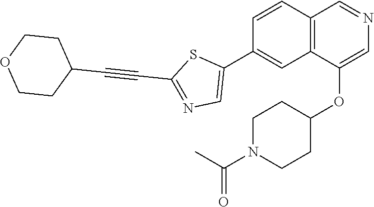 [SMILES: CC(=O)N1CCC(Oc2cncc3ccc(-c4cnc(C#CC5CCOCC5)s4)cc23)CC1] |
| 97 | 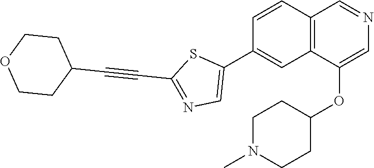 [SMILES: CN1CCC(Oc2cncc3ccc(-c4cnc(C#CC5CCOCC5)s4)cc23)CC1] |
| 98 | 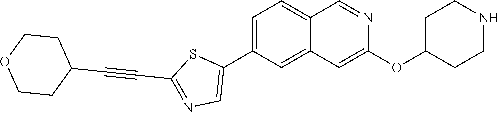 [SMILES: C(#CC1CCOCC1)c1ncc(-c2ccc3cnc(OC4CCNCC4)cc3c2)s1] |
| 99 | 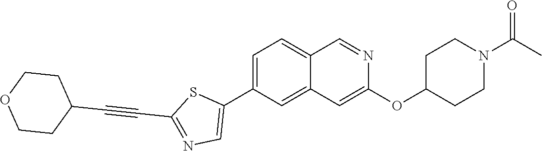 [SMILES: CC(=O)N1CCC(Oc2cc3cc(-c4cnc(C#CC5CCOCC5)s4)ccc3cn2)CC1] |
| 100 | 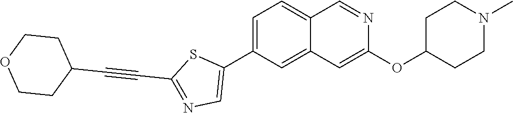 [SMILES: CN1CCC(Oc2cc3cc(-c4cnc(C#CC5CCOCC5)s4)ccc3cn2)CC1] |
| 101 | 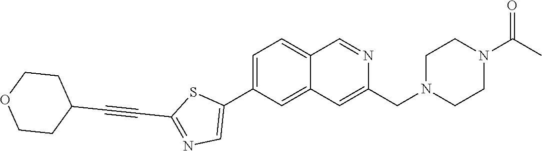 [SMILES: CC(=O)N1CCN(Cc2cc3cc(-c4cnc(C#CC5CCOCC5)s4)ccc3cn2)CC1] |
| 102 | 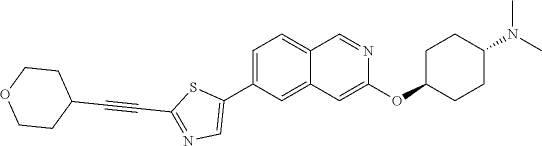 [SMILES: CN(C)[C@H]1CC[C@H](Oc2cc3cc(-c4cnc(C#CC5CCOCC5)s4)ccc3cn2)CC1] |
| 103 | 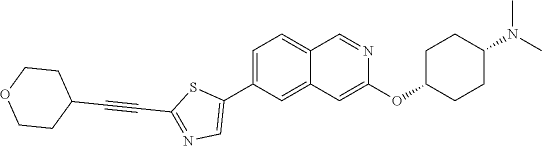 [SMILES: CN(C)[C@H]1CC[C@@H](Oc2cc3cc(-c4cnc(C#CC5CCOCC5)s4)ccc3cn2)CC1] |
| 104 | 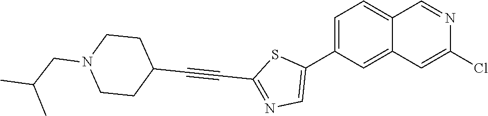 [SMILES: CC(C)CN1CCC(C#Cc2ncc(-c3ccc4cnc(Cl)cc4c3)s2)CC1] |
| 105 | 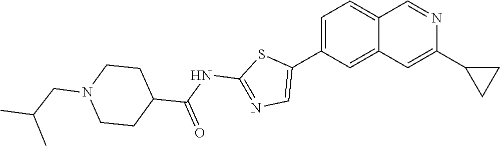 [SMILES: CC(C)CN1CCC(C(=O)Nc2ncc(-c3ccc4cnc(C5CC5)cc4c3)s2)CC1] |
| 106 | 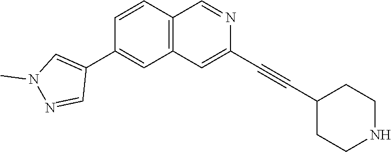 [SMILES: Cn1cc(-c2ccc3cnc(C#CC4CCNCC4)cc3c2)cn1] |
| 107 | |
| 108 | |
| 109 | |
| 110 | 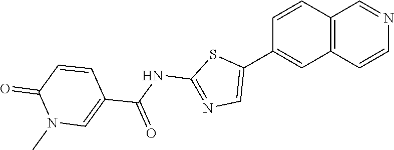 [SMILES: Cn1cc(C(=O)Nc2ncc(-c3ccc4cnccc4c3)s2)ccc1=O] |
| 111 | 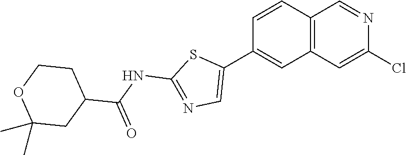 [SMILES: CC1(C)CC(C(=O)Nc2ncc(-c3ccc4cnc(Cl)cc4c3)s2)CCO1] |
| 112 | 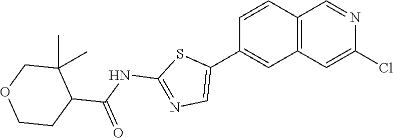 [SMILES: CC1(C)COCCC1C(=O)Nc1ncc(-c2ccc3cnc(Cl)cc3c2)s1] |
| 113 | 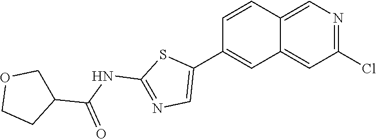 [SMILES: O=C(Nc1ncc(-c2ccc3cnc(Cl)cc3c2)s1)C1CCOC1] |
| 114 | 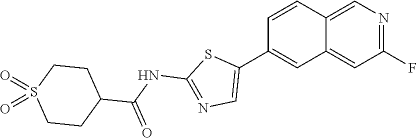 [SMILES: O=C(Nc1ncc(-c2ccc3cnc(F)cc3c2)s1)C1CCS(=O)(=O)CC1] |
| 115 | 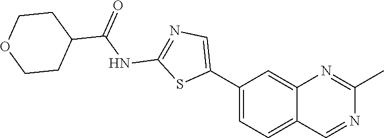 [SMILES: Cc1ncc2ccc(-c3cnc(NC(=O)C4CCOCC4)s3)cc2n1] |
| 116 | 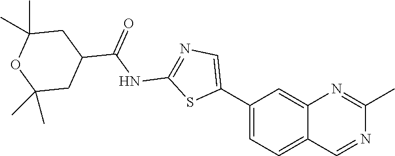 |

### Compounds of Formula (II)

The substituent groups used in this section (e.g., R¹, R², and the like) refer solely to the groups in Formula (II).

Some embodiments provide a compound of Formula (II):

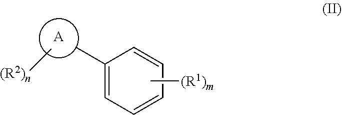

or a pharmaceutically acceptable salt thereof, wherein:
Ring A is a 5-14 membered heteroaryl or a 5-14 membered heterocyclyl;
each R¹ is independently halogen, hydroxyl, cyano, C1-C6 alkyl, C1-C6 alkoxy, —C(═O)OR^{A}, —NR^{B}R^{C}, and —C(═O)NR^{B}R^{C};
each R² is independently —C(═O)OR^{D}, C1-C6 alkyl, C2-C6 alkynyl optionally substituted with 4-8 membered heterocyclyl optionally substituted with C1-C6 alkyl, —C(═O)-phenyl, —(C1-C6 alkyl)-phenyl, —(C1-C6 alkyl)-4-10 membered heterocyclyl optionally substituted with C1-C6 alkyl, 4-10 membered heterocyclyl optionally substituted with C1-C6 alkyl or —CO₂C1-C6 alkyl, phenyl optionally substituted with cyano or fluoro, —NHC(═O)R^{E}, 5-6 membered heteroaryl optionally substituted with C1-C6 alkoxy,
m is 1, 2, or 3;
n is 0, 1, 2, or 3;
each R^{A}, R^{B}, R^{C}, and R^{D} is independently hydrogen or C1-C6 alkyl; and
each R^{E} is independently C3-C6 cycloalkyl, 4-8 membered heterocyclyl optionally substituted with C1-C6 alkyl, or 5-6 membered heteroaryl optionally substituted with C1-C6 alkyl.

In some embodiments, Ring A is a monocyclic heteroaryl or monocyclic heterocyclyl. In some embodiments, Ring A is a monocyclic heteroaryl. In some embodiments, Ring A is a monocyclic heterocyclyl.

In some embodiments, Ring A is a 5-6 membered heteroaryl. In some embodiments, Ring A is a 5-membered heteroaryl. In some embodiments, Ring A is thiazole or pyrazole. In some embodiments, Ring A is

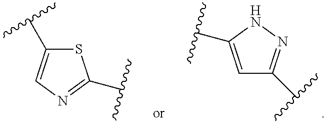 [SMILES: *c1cnc(*)s1 ; *c1cc(*)[nH]n1]

In some embodiments, Ring A is a 6-membered heteroaryl. In some embodiments, Ring A is pyridine or pyrimidin-4(3H)-one. In some embodiments, Ring A is

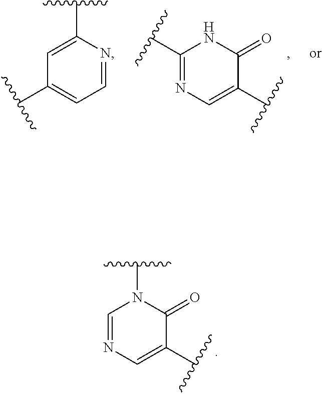 [SMILES: *c1ccnc(*)c1 ; *c1ncc(*)c(=O)[nH]1 ; *c1cncn(*)c1=O]

In some embodiments, Ring A is a bicyclic heteroaryl or a bicyclic heterocyclyl. In some embodiments, Ring A is a bicyclic heterocyclyl. In some embodiments, Ring A is a bicyclic heteroaryl. In some embodiments, Ring A is an 8-12 membered bicyclic heteroaryl or bicyclic 8-12 membered heterocyclyl. In some embodiments, Ring A is an 8-12 membered bicyclic heteroaryl. In some embodiments, Ring A is an bicyclic 8-12 membered heterocyclyl.

In some embodiments, Ring A is a bicyclic 9-10 membered heteroaryl or a bicyclic 9-10 membered heterocyclyl. In some embodiments, Ring A is a 9-membered bicyclic heteroaryl. In some embodiments, Ring A is pyrazolo[1,5-a]pyridine, 1H-pyrrolo[2,3-b]pyridine, pyrrolo[1,2-a]pyrazin-1(2H)-one, pyrazolo[1,5-a]pyrazine, imidazo[1,2-b]pyridazine, pyrazolo[1,5-a]pyrimidine, or 1,3-dihydro-2H-pyrrolo[2,3-b]pyridin-2-one. In some embodiments, Ring A is

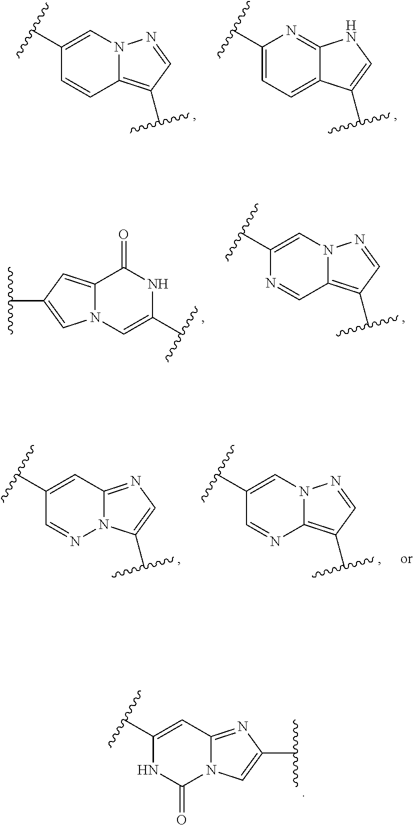

In some embodiments, Ring A is a bicyclic 9-membered heterocyclyl. In some embodiments, Ring A is 5,6,7,8-tetrahydroimidazo[1,2-a]pyrazine, 1,3-dihydro-2H-pyrrolo[2,3-b]pyridin-2-one, or 1,3-dihydro-2H-imidazo[4,5-b]pyridin-2-one. In some embodiments, Ring A

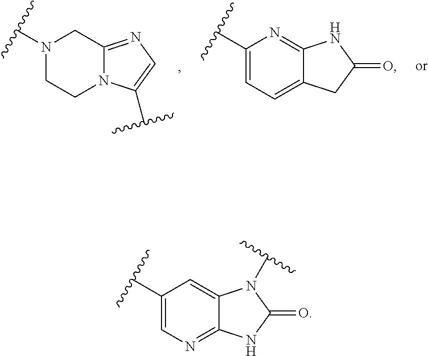 [SMILES: *c1cnc2n1CCN(*)C2 ; *c1ccc2c(n1)NC(=O)C2 ; "or" ; *c1cnc2[nH]c(=O)n(*)c2c1]

In some embodiments, Ring A is a tricyclic heteroaryl or a tricyclic heterocyclyl. In some embodiments, Ring A is a tricyclic heteroaryl. In some embodiments, Ring A is a tricyclic heterocyclyl. In some embodiments, Ring A is a 10-14 membered tricyclic heteroaryl or a 10-14 membered tricyclic heterocyclyl. In some embodiments, Ring A is a 10-14 membered tricyclic heteroaryl. In some embodiments, Ring A is a 10-14 membered tricyclic heterocyclyl. In some embodiments, Ring A is a 11-13 membered tricyclic heteroaryl or a 11-13 membered tricyclcic heterocyclyl. In some embodiments, Ring A is a 11-13 membered tricyclic heteroaryl. In some embodiments, Ring A is a 11-13 membered tricyclcic heterocyclyl. In some embodiments, Ring A is a 12-membered tricyclic heteroaryl. In some embodiments, Ring A is 8H-pyrazolo[1,5-a]pyrrolo[3,2-e]pyrimidine. In some embodiments, Ring A is

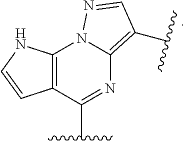 [SMILES: *c1nc2c(*)cnn2c2[nH]ccc12]

In some embodiments, Ring A is a 12-membered tricyclic heterocyclyl. In some embodiments, Ring A is 7,8,9,10-tetrahydro-pyrazolo[5,1-f][1,6]naphthyridine, or 7,8-dihydro-6H-pyrazolo[1,5-a]pyrrolo[3,2-e]pyrimidine. In some embodiments, Ring A is

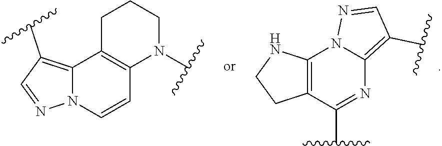 [SMILES: *c1cnn2ccc3c(c12)CCCN3* ; *c1nc2c(*)cnn2c2c1CCN2]

In some embodiments, m is 1 or 2. In some embodiments, m is 1. In some embodiments, m is 2. In some embodiments, m is 3.

In some embodiments, R¹ is halogen. In some embodiments, R¹ is fluoro. In some embodiments, R¹ is chloro. In some embodiments, R¹ is hydroxyl. In some embodiments, R¹ is cyano.

In some embodiments, R¹ is C1-C6 alkyl. In some embodiments, R¹ is C1-C3 alkyl. In some embodiments, R¹ is methyl.

In some embodiments, R¹ is C1-C6 alkoxy. In some embodiments, R¹ is C1-C3 alkoxy.

In some embodiments, R¹ is methoxy.

In some embodiments, R¹ is —C(═O)OR^{A}. In some embodiments, R¹ is —C(═O)OH, —C(═O)OCH₂CH₃, or —C(═O)OC(CH₃)₃.

In some embodiments, R¹ is —NR^{B}R^{C}. In some embodiments, R¹ is —NH₂, —NHCH₃, or —NH(CH₃)₂.

In some embodiments, R¹ is —C(═O)NR^{B}R^{C}. In some embodiments, R¹ is —C(═O)NH₂, or —C(═O)NHCH₃.

In some embodiments, one of R¹ is C1-C6 alkyl and the other R¹ is —C(═O)OR^{A}. In some embodiments, one of R¹ is C1-C6 alkyl and the other R¹ is —C(═O)OH or —C(═O)OCH₃). In some embodiments, one of R¹ is methyl and the other R¹ is —C(═O)OH or —C(═O)OCH₃).

In some embodiments, one of R¹ is cyano and the other R¹ is halogen or hydroxyl. In some embodiments, one of R¹ is cyano and the other R¹ is C1-C6 alkoxy. In some embodiments, one of R¹ is cyano and the other R¹ is —OCH(CH₃)₂).

In some embodiments, n is 0. In some embodiments, n is 1. In some embodiments, n is 1, 2, or 3. In some embodiments, n is 2. In some embodiments, n is 3.

In some embodiments, R² is —C(═O)OR^{D}. In some embodiments, R² is —C(═O)OCH(CH₃)₃.

In some embodiments, R² is C1-C6 alkyl.

In some embodiments, R² is C2-C6 alkynyl optionally substituted with 4-8 membered heterocyclyl optionally substituted with C1-C6 alkyl. In some embodiments, R² is C2-C6 alkynyl substituted with 4-8 membered heterocyclyl optionally substituted with C1-C6 alkyl. In some embodiments, R² is C2-C6 alkynyl substituted with 4-8 membered heterocyclyl substituted with C1-C6 alkyl. In some embodiments, R² is C2-C6 alkynyl substituted with an unsubstituted 4-8 membered heterocyclyl. In some embodiments, R² is an unsubstituted C2-C6 alkynyl.

In some embodiments, R² is

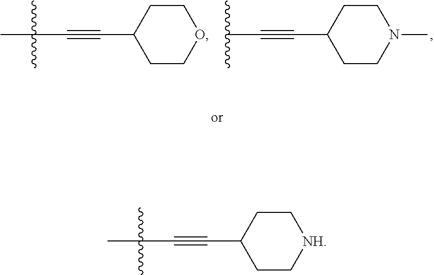

In some embodiments, R² is —C(═O)-phenyl.

In some embodiments, R² is —(C1-C6 alkyl)-phenyl. In some embodiments, R² is

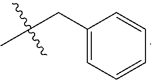 [SMILES: *Cc1ccccc1]

In some embodiments, R² is —(C1-C6 alkyl)-4-10 membered heterocyclyl optionally substituted with C1-C6 alkyl. In some embodiments, R² is —(C1-C6 alkyl)-4-10 membered heterocyclyl substituted with C1-C6 alkyl. In some embodiments, R² is —(C1-C6 alkyl)-4-10 membered heterocyclyl substituted with methyl. In some embodiments, R² is —(C1-C6 alkyl)-4-10 membered heterocyclyl.

In some embodiments, R² is —(C1-C3 alkyl)-4-6 membered heterocyclyl optionally substituted with C1-C6 alkyl. In some embodiments, R² is —(C1-C3 alkyl)-4-6 membered heterocyclyl substituted with C1-C6 alkyl. In some embodiments, R² is —(C1-C3 alkyl)-4-6 membered heterocyclyl substituted with methyl. In some embodiments, R² is —(C1-C3 alkyl)-4-6 membered heterocyclyl.

In some embodiments, R² is

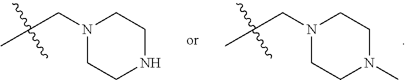

In some embodiments, R² is 4-10 membered heterocyclyl optionally substituted with C1-C6 alkyl or —CO₂C1-C6 alkyl. In some embodiments, R² is 4-10 membered heterocyclyl substituted with C1-C6 alkyl or —CO₂C1-C6 alkyl. In some embodiments, R² is 4-10 membered heterocyclyl substituted with C1-C6 alkyl. In some embodiments, R² is 4-10 membered heterocyclyl substituted with methyl. In some embodiments, R² is 4-10 membered heterocyclyl substituted with —CO₂C1-C6 alkyl. In some embodiments, R² is 4-10 membered heterocyclyl substituted with —CO₂CH₃. In some embodiments, R² is an unsubstituted 4-10 membered heterocyclyl.

In some embodiments, R² is

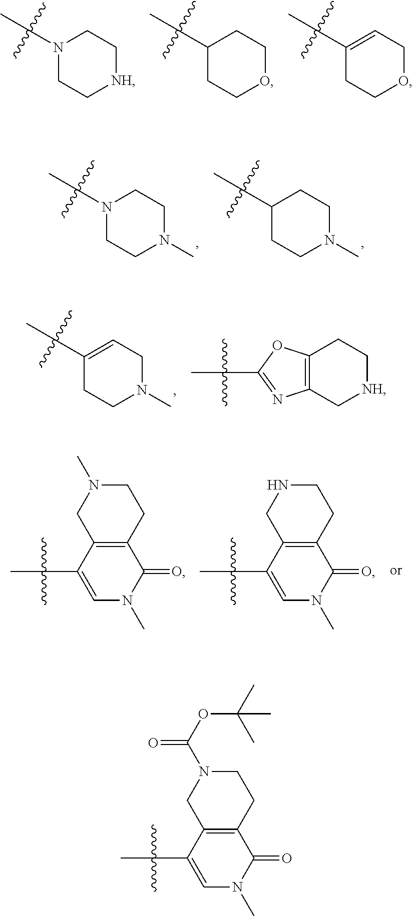

In some embodiments, R² is phenyl optionally substituted with cyano or fluoro. In some embodiments, R² is phenyl substituted with cyano or fluoro. In some embodiments, R² is phenyl substituted with cyano. In some embodiments, R² is phenyl substituted with fluoro.

In some embodiments, R² is

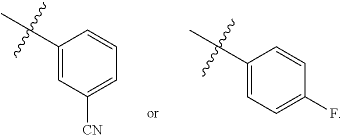 [SMILES: *c1cccc(C#N)c1 ; *c1ccc(F)cc1]

In some embodiments, R² is —NHC(═O)R^{E}.

In some embodiments, R² is

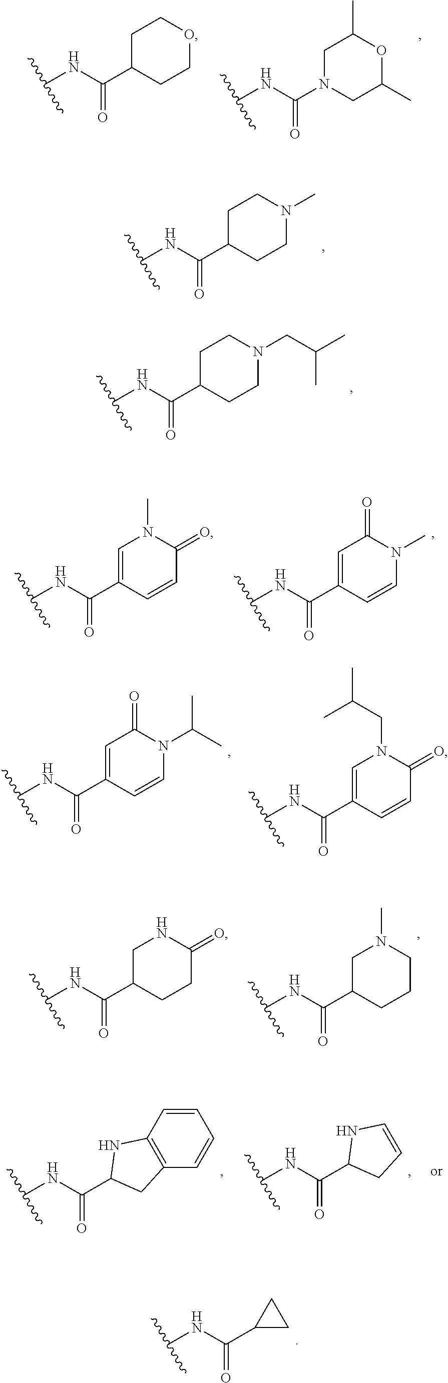

In some embodiments, R² is 5-6 membered heteroaryl optionally substituted with C1-C6 alkoxy. In some embodiments, R² is 5-6 membered heteroaryl substituted with C1-C6 alkoxy. In some embodiments, R² is 5-6 membered heteroaryl substituted with methoxy or ethoxy. In some embodiments, R² is an unsubstituted 5-6 membered heteroaryl.

In some embodiments, R² is

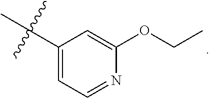 [SMILES: *c1ccnc(OCC)c1]

In some embodiments, R^{A} is hydrogen or C1-C6 alkyl. In some embodiments, R^{A} is hydrogen or methyl. In some embodiments, R^{A} is hydrogen. In some embodiments, R^{A} is C1-C6 alkyl. In some embodiments, R^{A} is methyl. In some embodiments, R^{B} is hydrogen or C1-C6 alkyl. In some embodiments, R^{B} is hydrogen or methyl. In some embodiments, R^{B} is hydrogen. In some embodiments, R^{B} is C1-C6 alkyl. In some embodiments, R^{B} is methyl.

In some embodiments, R^{A} and R^{B} are the same. In some embodiments, R^{A} and R^{B} are different. In some embodiments, one of R^{A} and R^{B} is hydrogen and the other of R^{A} and R^{B} is C1-C6 alkyl. In some embodiments, one of R^{A} and R^{B} is hydrogen and the other of R^{A} and R^{B} is methyl. In some embodiments, R^{A} and R^{B} are each hydrogen. In some embodiments, R^{A} and R^{B} are each C1-C6 alkyl. In some embodiments, R^{A} and R^{B} are each methyl.

In some embodiments, R^{C} is hydrogen or C1-C6 alkyl. In some embodiments, R^{C} is hydrogen or methyl. In some embodiments, R^{C} is hydrogen. In some embodiments, R^{C} is C1-C6 alkyl. In some embodiments, R^{C} is methyl. In some embodiments, R^{D} is hydrogen or C1-C6 alkyl. In some embodiments, R^{D} is hydrogen or methyl. In some embodiments, R^{D} is hydrogen. In some embodiments, R^{D} is C1-C6 alkyl. In some embodiments, R^{D} is methyl.

In some embodiments, R^{C} and R^{D} are the same. In some embodiments, R^{C} and R^{D} are different. In some embodiments, one of R^{C} and R^{D} is hydrogen and the other of R^{C} and R^{D} is C1-C6 alkyl. In some embodiments, one of R^{C} and R^{D} is hydrogen and the other of R^{C} and R^{D} is methyl. In some embodiments, R^{C} and R^{D} are each hydrogen. In some embodiments, R^{C} and R^{D} are each C1-C6 alkyl. In some embodiments, R^{C} and R^{D} are each methyl.

In some embodiments, R^{E} is a C3-C6 cycloalkyl.

In some embodiments, R^{E} is 4-8 membered heterocyclyl optionally substituted with C1-C6 alkyl. In some embodiments, R^{E} is 4-8 membered heterocyclyl substituted with C1-C6 alkyl. In some embodiments, R^{E} is 4-8 membered heterocyclyl substituted with methyl. In some embodiments, R^{E} is an unsubstituted 4-8 membered heterocyclyl.

In some embodiments, R^{E} is 5-6 membered heteroaryl optionally substituted with C1-C6 alkyl. In some embodiments, R^{E} is 5-6 membered heteroaryl substituted with C1-C6 alkyl. In some embodiments, R^{E} is 5-6 membered heteroaryl substituted with methyl. In some embodiments, R^{E} is an unsubstituted 5-6 membered heteroaryl.

In some embodiments, the compound of Formula (II), or a pharmaceutically acceptable salt thereof, is a compound of formula (II-1), or a pharmaceutically acceptable salt thereof:

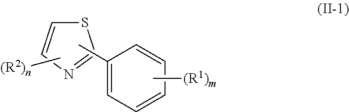

In some embodiments, the compound of Formula (II), or a pharmaceutically acceptable salt thereof, is a compound of formula (II-1-i), or a pharmaceutically acceptable salt thereof:

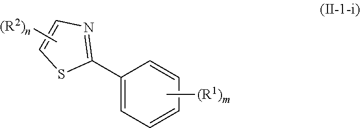

In some embodiments, the compound of Formula (II), or a pharmaceutically acceptable salt thereof, is a compound of formula (II-1-ii), or a pharmaceutically acceptable salt thereof:

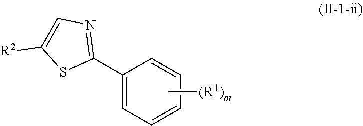

In some embodiments, the compound of Formula (II), or a pharmaceutically acceptable salt thereof, is a compound of formula (II-1-iii), or a pharmaceutically acceptable salt thereof:

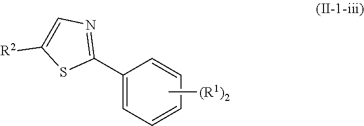

In some embodiments, the compound of Formula (II), or a pharmaceutically acceptable salt thereof, is a compound of formula (II-1-iv), or a pharmaceutically acceptable salt thereof:

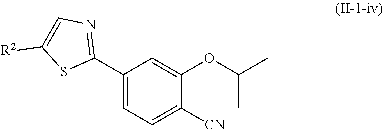

In some embodiments, the compound of Formula (II), or a pharmaceutically acceptable salt thereof, is a compound of formula (II-2), or a pharmaceutically acceptable salt thereof:

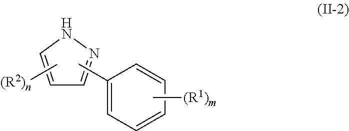

In some embodiments, the compound of Formula (II), or a pharmaceutically acceptable salt thereof, is a compound of formula (II-3), or a pharmaceutically acceptable salt thereof:

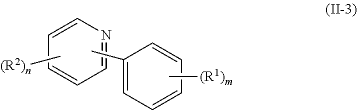

In some embodiments, the compound of Formula (II), or a pharmaceutically acceptable salt thereof, is a compound of formula (II-4), or a pharmaceutically acceptable salt thereof:

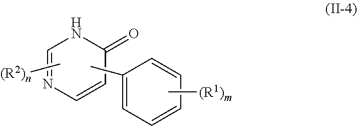

In some embodiments, the compound of Formula (II), or a pharmaceutically acceptable salt thereof, is a compound of formula (II-5), or a pharmaceutically acceptable salt thereof:

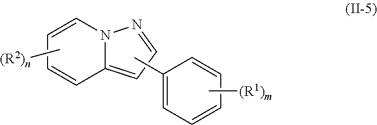

In some embodiments, the compound of Formula (II), or a pharmaceutically acceptable salt thereof, is a compound of formula (II-6), or a pharmaceutically acceptable salt thereof:

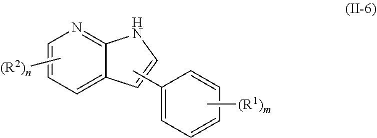

In some embodiments, the compound of Formula (II), or a pharmaceutically acceptable salt thereof, is a compound of formula (II-7), or a pharmaceutically acceptable salt thereof:

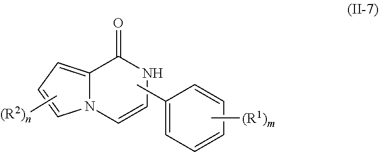

In some embodiments, the compound of Formula (II), or a pharmaceutically acceptable salt thereof, is a compound of formula (II-8), or a pharmaceutically acceptable salt thereof:

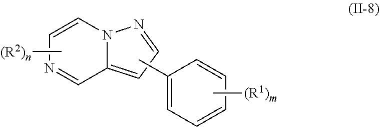

In some embodiments, the compound of Formula (II), or a pharmaceutically acceptable salt thereof, is a compound of formula (II-9), or a pharmaceutically acceptable salt thereof:

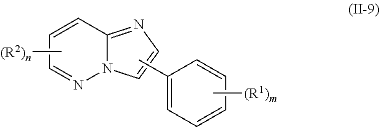

In some embodiments, the compound of Formula (II), or a pharmaceutically acceptable salt thereof, is a compound of formula (II-10), or a pharmaceutically acceptable salt thereof:

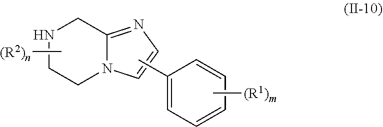

In some embodiments, the compound of Formula (II), or a pharmaceutically acceptable salt thereof, is a compound of formula (II-11), or a pharmaceutically acceptable salt thereof:

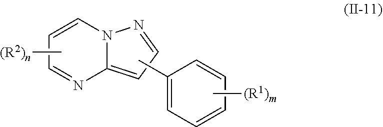

In some embodiments, the compound of Formula (II), or a pharmaceutically acceptable salt thereof, is a compound of formula (II-12), or a pharmaceutically acceptable salt thereof:

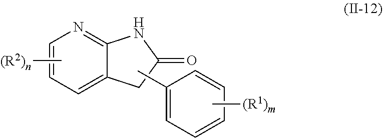

In some embodiments, the compound of Formula (II), or a pharmaceutically acceptable salt thereof, is a compound of formula (II-13), or a pharmaceutically acceptable salt thereof:

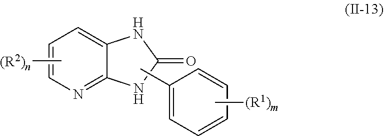

In some embodiments, the compound of Formula (II), or a pharmaceutically acceptable salt thereof, is a compound of formula (II-14), or a pharmaceutically acceptable salt thereof:

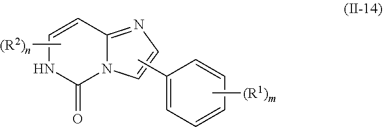

In some embodiments, the compound of Formula (II), or a pharmaceutically acceptable salt thereof, is a compound of formula (II-15), or a pharmaceutically acceptable salt thereof:

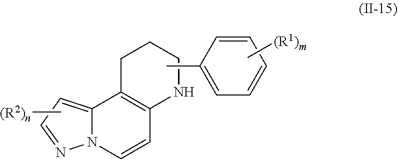

In some embodiments, the compound of Formula (II), or a pharmaceutically acceptable salt thereof, is a compound of formula (II-16), or a pharmaceutically acceptable salt thereof:

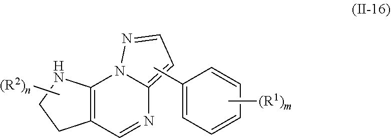

In some embodiments, the compound of Formula (II), or a pharmaceutically acceptable salt thereof, is a compound of formula (II-17), or a pharmaceutically acceptable salt thereof:

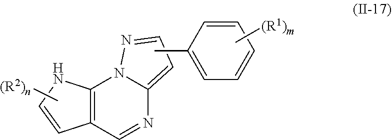

In some embodiments, the compound of Formula (II) is selected from a compound in Table 2, or a pharmaceutically acceptable salt thereof.

TABLE 2
Selected Compounds of Formula (II)

| Compound | |
|---|---|
| No. | Structure |
| 1 | 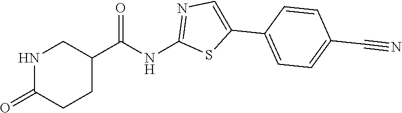 [SMILES: N#Cc1ccc(-c2cnc(NC(=O)C3CCC(=O)NC3)s2)cc1] |
| 2 | 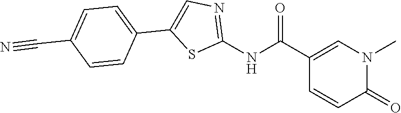 [SMILES: Cn1cc(C(=O)Nc2ncc(-c3ccc(C#N)cc3)s2)ccc1=O] |
| 3 | 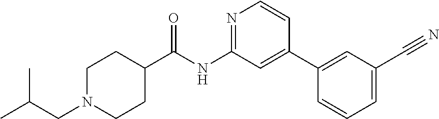 [SMILES: CC(C)CN1CCC(C(=O)Nc2cc(-c3cccc(C#N)c3)ccn2)CC1] |
| 4 | 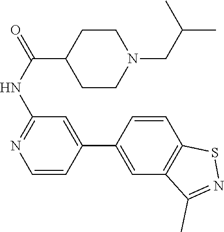 [SMILES: Cc1nsc2ccc(-c3ccnc(NC(=O)C4CCN(CC(C)C)CC4)c3)cc12] |
| 5 | 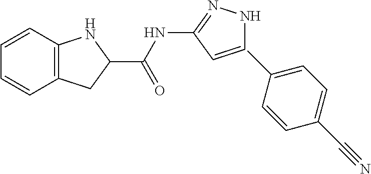 |
| 6 | 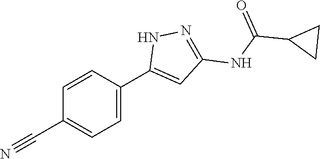 [SMILES: N#Cc1ccc(-c2cc(NC(=O)C3CC3)n[nH]2)cc1] |
| 7 | 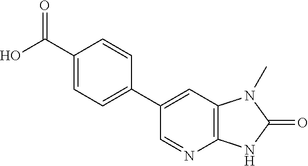 [SMILES: Cn1c(=O)[nH]c2ncc(-c3ccc(C(=O)O)cc3)cc21] |
| 8 | 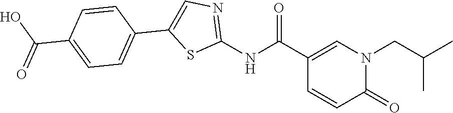 [SMILES: CC(C)Cn1cc(C(=O)Nc2ncc(-c3ccc(C(=O)O)cc3)s2)ccc1=O] |
| 9 | 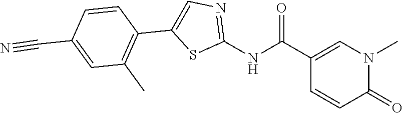 [SMILES: Cc1cc(C#N)ccc1-c1cnc(NC(=O)c2ccc(=O)n(C)c2)s1] |
| 10 | 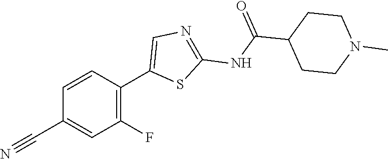 [SMILES: CN1CCC(C(=O)Nc2ncc(-c3ccc(C#N)cc3F)s2)CC1] |
| 11 | 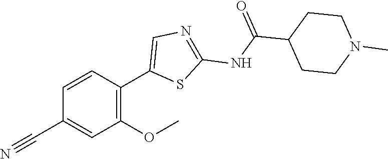 [SMILES: COc1cc(C#N)ccc1-c1cnc(NC(=O)C2CCN(C)CC2)s1] |
| 12 | 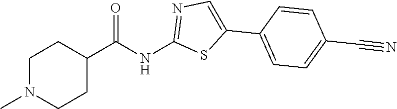 [SMILES: CN1CCC(C(=O)Nc2ncc(-c3ccc(C#N)cc3)s2)CC1] |
| 13 | 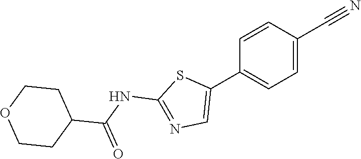 [SMILES: N#Cc1ccc(-c2cnc(NC(=O)C3CCOCC3)s2)cc1] |
| 14 | 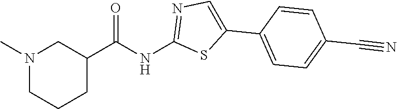 [SMILES: CN1CCCC(C(=O)Nc2ncc(-c3ccc(C#N)cc3)s2)C1] |
| 15 | 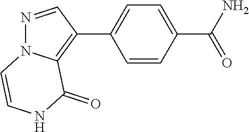 [SMILES: NC(=O)c1ccc(-c2cnn3cc[nH]c(=O)c23)cc1] |
| 16 | 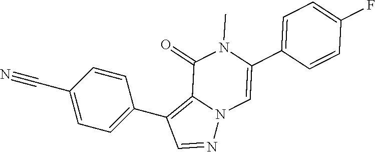 [SMILES: Cn1c(-c2ccc(F)cc2)cn2ncc(-c3ccc(C#N)cc3)c2c1=O] |
| 17 | 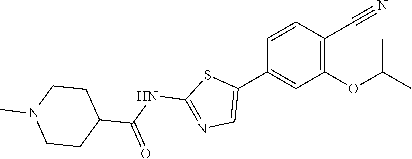 [SMILES: CC(C)Oc1cc(-c2cnc(NC(=O)C3CCN(C)CC3)s2)ccc1C#N] |
| 18 | 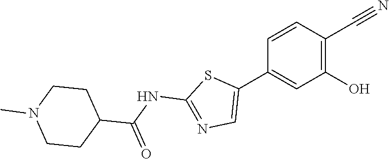 [SMILES: CN1CCC(C(=O)Nc2ncc(-c3ccc(C#N)c(O)c3)s2)CC1] |
| 19 | 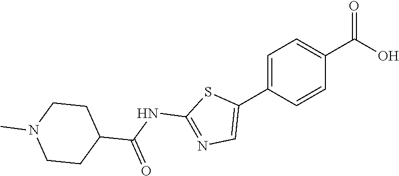 [SMILES: CN1CCC(C(=O)Nc2ncc(-c3ccc(C(=O)O)cc3)s2)CC1] |
| 20 | 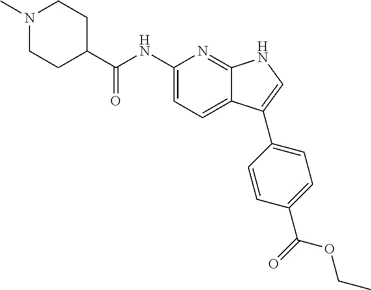 [SMILES: CCOC(=O)c1ccc(-c2c[nH]c3nc(NC(=O)C4CCN(C)CC4)ccc23)cc1] |
| 21 | 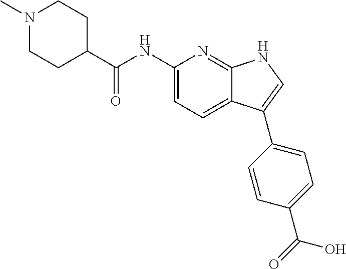 [SMILES: CN1CCC(C(=O)Nc2ccc3c(-c4ccc(C(=O)O)cc4)c[nH]c3n2)CC1] |
| 22 | 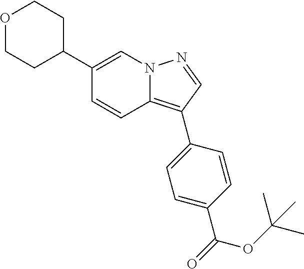 [SMILES: CC(C)(C)OC(=O)c1ccc(-c2cnn3cc(C4CCOCC4)ccc23)cc1] |
| 23 | 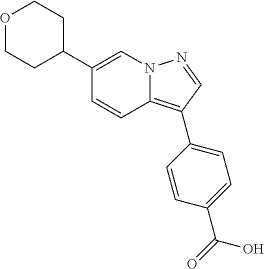 [SMILES: O=C(O)c1ccc(-c2cnn3cc(C4CCOCC4)ccc23)cc1] |
| 24 | 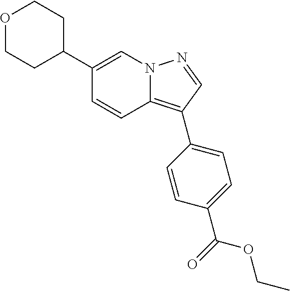 [SMILES: CCOC(=O)c1ccc(-c2cnn3cc(C4CCOCC4)ccc23)cc1] |
| 25 | 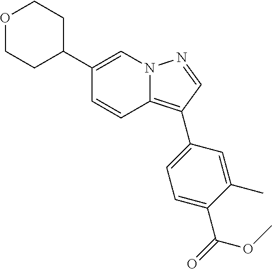 [SMILES: COC(=O)c1ccc(-c2cnn3cc(C4CCOCC4)ccc23)cc1C] |
| 26 | 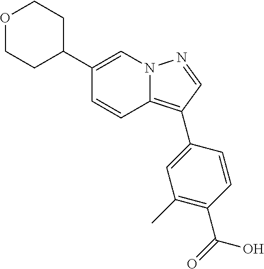 [SMILES: Cc1cc(-c2cnn3cc(C4CCOCC4)ccc23)ccc1C(=O)O] |
| 27 | 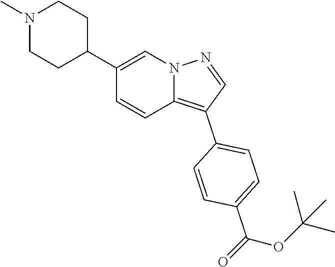 [SMILES: CN1CCC(c2ccc3c(-c4ccc(C(=O)OC(C)(C)C)cc4)cnn3c2)CC1] |
| 28 | 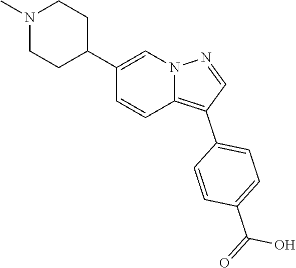 [SMILES: CN1CCC(c2ccc3c(-c4ccc(C(=O)O)cc4)cnn3c2)CC1] |
| 29 | 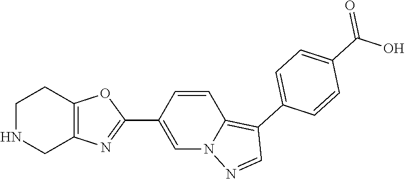 [SMILES: O=C(O)c1ccc(-c2cnn3cc(-c4nc5c(o4)CCNC5)ccc23)cc1] |
| 30 | 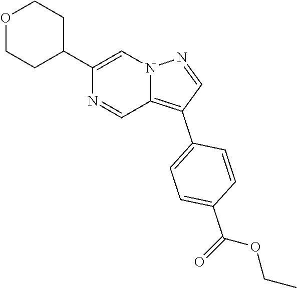 [SMILES: CCOC(=O)c1ccc(-c2cnn3cc(C4CCOCC4)ncc23)cc1] |
| 31 | 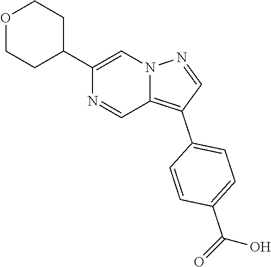 [SMILES: O=C(O)c1ccc(-c2cnn3cc(C4CCOCC4)ncc23)cc1] |
| 32 | 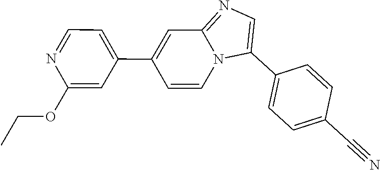 [SMILES: CCOc1cc(-c2ccn3c(-c4ccc(C#N)cc4)cnc3c2)ccn1] |
| 33 | 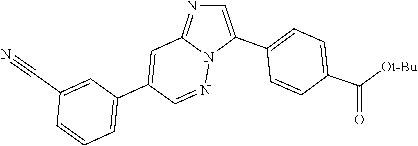 [SMILES: CC(C)(C)OC(=O)c1ccc(-c2cnc3cc(-c4cccc(C#N)c4)cnn23)cc1] |
| 34 | 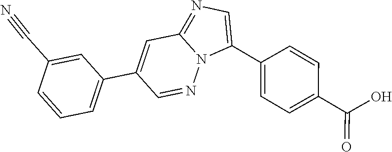 [SMILES: N#Cc1cccc(-c2cnn3c(-c4ccc(C(=O)O)cc4)cnc3c2)c1] |
| 35 | 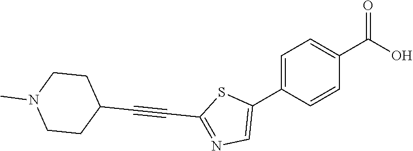 [SMILES: CN1CCC(C#Cc2ncc(-c3ccc(C(=O)O)cc3)s2)CC1] |
| 36 | 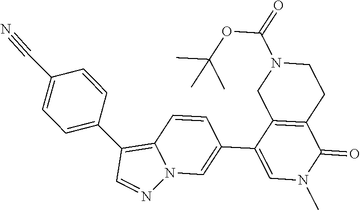 [SMILES: Cn1cc(-c2ccc3c(-c4ccc(C#N)cc4)cnn3c2)c2c(c1=O)CCN(C(=O)OC(C)(C)C)C2] |
| 37 | 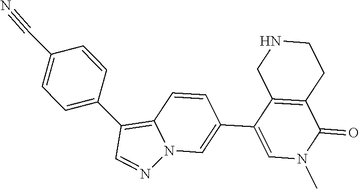 [SMILES: Cn1cc(-c2ccc3c(-c4ccc(C#N)cc4)cnn3c2)c2c(c1=O)CCNC2] |
| 38 | 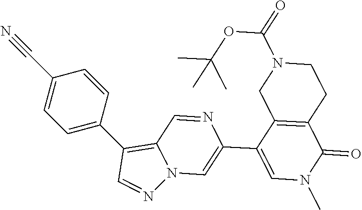 [SMILES: Cn1cc(-c2cn3ncc(-c4ccc(C#N)cc4)c3cn2)c2c(c1=O)CCN(C(=O)OC(C)(C)C)C2] |
| 39 | 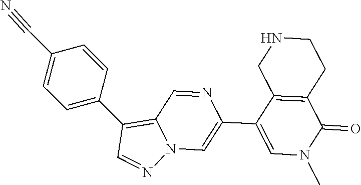 [SMILES: Cn1cc(-c2cn3ncc(-c4ccc(C#N)cc4)c3cn2)c2c(c1=O)CCNC2] |
| 40 | 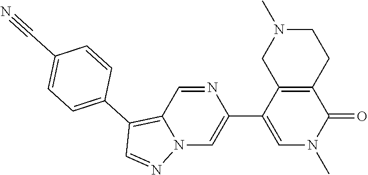 [SMILES: CN1CCc2c(c(-c3cn4ncc(-c5ccc(C#N)cc5)c4cn3)cn(C)c2=O)C1] |
| 41 | 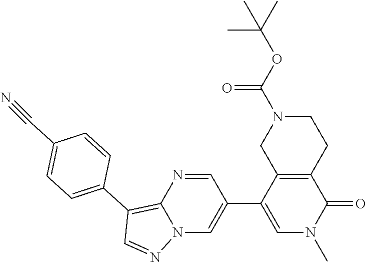 [SMILES: Cn1cc(-c2cnc3c(-c4ccc(C#N)cc4)cnn3c2)c2c(c1=O)CCN(C(=O)OC(C)(C)C)C2] |
| 42 | 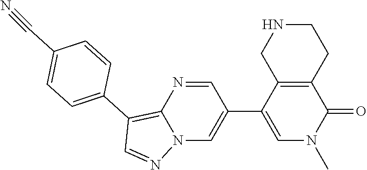 [SMILES: Cn1cc(-c2cnc3c(-c4ccc(C#N)cc4)cnn3c2)c2c(c1=O)CCNC2] |
| 43 | 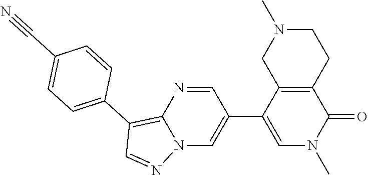 [SMILES: CN1CCc2c(c(-c3cnc4c(-c5ccc(C#N)cc5)cnn4c3)cn(C)c2=O)C1] |
| 44 | 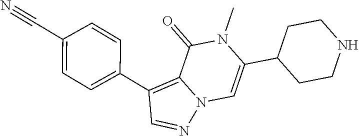 [SMILES: Cn1c(C2CCNCC2)cn2ncc(-c3ccc(C#N)cc3)c2c1=O] |
| 45 | 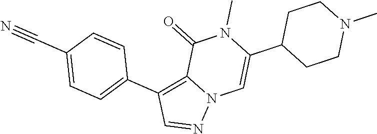 [SMILES: CN1CCC(c2cn3ncc(-c4ccc(C#N)cc4)c3c(=O)n2C)CC1] |
| 46 | 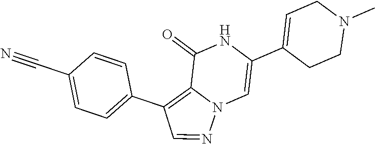 [SMILES: CN1CC=C(c2cn3ncc(-c4ccc(C#N)cc4)c3c(=O)[nH]2)CC1] |
| 47 | 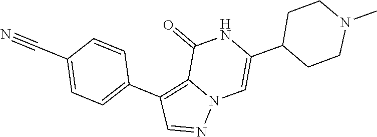 [SMILES: CN1CCC(c2cn3ncc(-c4ccc(C#N)cc4)c3c(=O)[nH]2)CC1] |
| 48 | 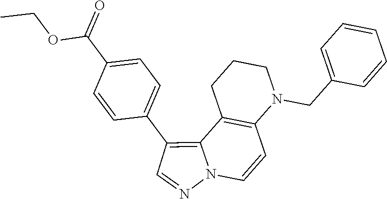 [SMILES: CCOC(=O)c1ccc(-c2cnn3ccc4c(c23)CCCN4Cc2ccccc2)cc1] |
| 49 | 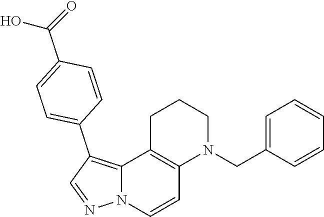 [SMILES: O=C(O)c1ccc(-c2cnn3ccc4c(c23)CCCN4Cc2ccccc2)cc1] |
| 50 | 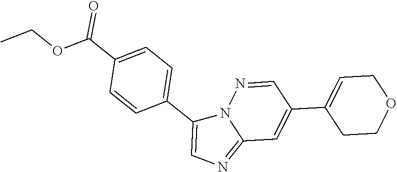 [SMILES: CCOC(=O)c1ccc(-c2cnc3cc(C4=CCOCC4)cnn23)cc1] |
| 51 | 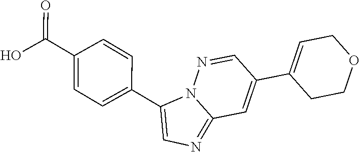 [SMILES: O=C(O)c1ccc(-c2cnc3cc(C4=CCOCC4)cnn23)cc1] |
| 52 | 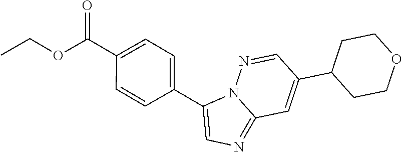 [SMILES: CCOC(=O)c1ccc(-c2cnc3cc(C4CCOCC4)cnn23)cc1] |
| 53 | 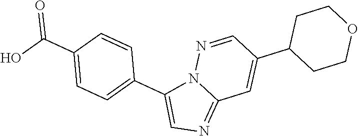 [SMILES: O=C(O)c1ccc(-c2cnc3cc(C4CCOCC4)cnn23)cc1] |
| 54 | 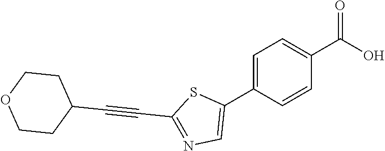 [SMILES: O=C(O)c1ccc(-c2cnc(C#CC3CCOCC3)s2)cc1] |
| 55 | 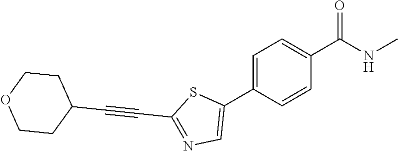 [SMILES: CNC(=O)c1ccc(-c2cnc(C#CC3CCOCC3)s2)cc1] |
| 56 | 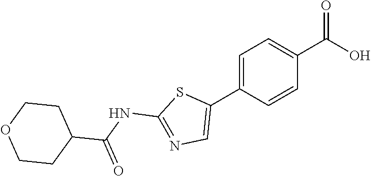 [SMILES: O=C(O)c1ccc(-c2cnc(NC(=O)C3CCOCC3)s2)cc1] |
| 57 | 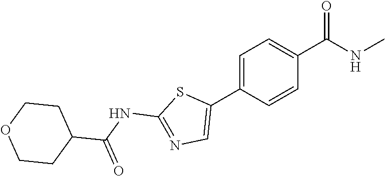 [SMILES: CNC(=O)c1ccc(-c2cnc(NC(=O)C3CCOCC3)s2)cc1] |
| 58 | 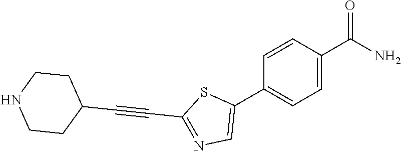 [SMILES: NC(=O)c1ccc(-c2cnc(C#CC3CCNCC3)s2)cc1] |
| 59 | 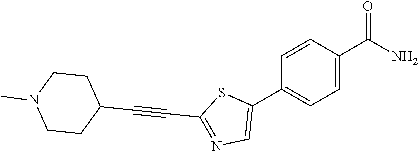 [SMILES: CN1CCC(C#Cc2ncc(-c3ccc(C(N)=O)cc3)s2)CC1] |
| 60 | 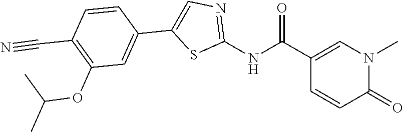 [SMILES: CC(C)Oc1cc(-c2cnc(NC(=O)c3ccc(=O)n(C)c3)s2)ccc1C#N] |
| 61 | 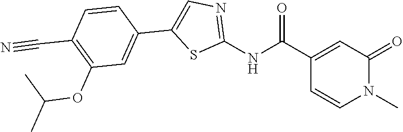 [SMILES: CC(C)Oc1cc(-c2cnc(NC(=O)c3ccn(C)c(=O)c3)s2)ccc1C#N] |
| 62 | 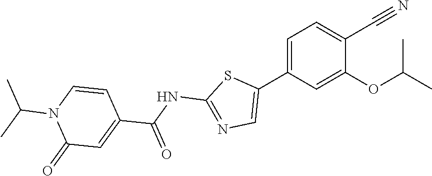 [SMILES: CC(C)Oc1cc(-c2cnc(NC(=O)c3ccn(C(C)C)c(=O)c3)s2)ccc1C#N] |
| 63 | 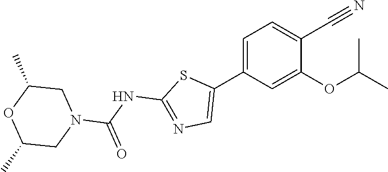 [SMILES: CC(C)Oc1cc(-c2cnc(NC(=O)N3C[C@@H](C)O[C@@H](C)C3)s2)ccc1C#N] |

### Compounds of Formula (III)

The substituent groups used in this section (e.g., R¹, R², and the like) refer solely to the groups in Formula (III).

Some embodiments provide a compound of Formula (III):

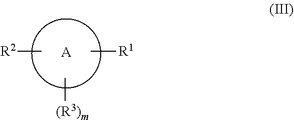

or a pharmaceutically acceptable salt thereof:
Ring A is 5-6 membered heteroaryl or 5-6 membered heterocyclyl;
R¹ is —NHC(═O)(C1-C6 alkylene)ₙR^{A}, phenyl optionally substituted with —NR^{F}R^{G}, -Q-R^{C}, or

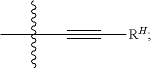

R² is C3-C6 cycloalkyl optionally substituted with —CO₂R^{B}, 5-10 membered heteroaryloxy, —(C1-C6 alkylene)ₚ-5-10 membered heteroaryl optionally substituted with C1-C6 alkyl, cyano, or 4-6 membered heterocyclyl; —(C1-C6 alkylene)ₜ-phenyl optionally substituted with cyano or —NR^{D}R^{E}; 4-6 membered heterocyclyl optionally substituted with C1-C6 alkyl;
R³ is C1-C6 alkyl;
R^{A} is 4-6 membered heterocyclyl optionally substituted with C1-C6 alkyl, or 5-10 membered heteroaryl optionally substituted with C1-C6 alkoxy or C1-C6 alkyl,
R^{B} is hydrogen or C1-C6 alkyl;
R^{C} is 4-10 membered heterocyclyl, 5-10 membered heteroaryl, or phenyl optionally substituted with —(C1-C6 alkylene)-NR^{D}R^{E};
R^{D}, R^{E}, and R^{F} are independently hydrogen, C1-C6 alkyl, or C3-C6 cycloalkyl;
R^{G} is hydrogen, C1-C6 alkyl, —C(═O)—C1-C6 alkyl, or —C(═O)—C3-C6 cycloalkyl;
R^{H} is 4-6 membered heterocyclyl optionally substituted with 1-2 independently selected C1-C6 alkyl;
Q is C1-C6 alkylene, NH, or O;
m is 0 or 1;
n is 0 or 1;
p is 0 or 1; and
t is 0 or 1.

In some embodiments, R¹ is —NHC(═O)(C1-C6 alkylene)ₙR^{A}. In some embodiments, R¹ is —NHC(═O)(C1-C2 alkylene)ₙR^{A}. In some embodiments, R¹ is —NHC(═O)R^{A}.

In some embodiments, n is 1. In some embodiments, n is 0.

In some embodiments, R^{A} is 4-6 membered heterocyclyl optionally substituted with C1-C6 alkyl. In some embodiments, R^{A} is 4-6 membered heterocyclyl substituted with C1-C6 alkyl. In some embodiments, R^{A} is oxetanyl, azetidinyl, pyrrolidinyl, pyrrolidinonyl, imidazolidinyl, piperidinyl, piperazinyl, tetrahydropyranyl, tetrahydrofuranyl, or morpholinyl; each optionally substituted with C1-C6 alkyl. In some embodiments, R^{A} is piperidinyl, tetrahydropyranyl, or tetrahydrofuranyl; each optionally substituted with C1-C6 alkyl. In some embodiments, R^{A} is oxetanyl, azetidinyl, pyrrolidinyl, pyrrolidinonyl, imidazolidinyl, piperidinyl, piperazinyl, or morpholinyl; each optionally substituted with C1-C6 alkyl. In some embodiments, R^{A} is oxetanyl, azetidinyl, pyrrolidinyl, pyrrolidinonyl, imidazolidinyl, piperidinyl, piperazinyl, or morpholinyl; each substituted with C1-C6 alkyl. In some embodiments, R^{A} is an unsubstituted 4-6 membered heterocyclyl.

In some embodiments, R^{A} is 5-10 membered heteroaryl optionally substituted with C1-C6 alkoxy or C1-C6 alkyl. In some embodiments, R^{A} is 5-6 membered heteroaryl optionally substituted with C1-C6 alkoxy or C1-C6 alkyl. In some embodiments, R^{A} is 5-10 membered heteroaryl substituted with C1-C6 alkoxy or C1-C6 alkyl. In some embodiments, R^{A} is 5-6 membered heteroaryl substituted with C1-C6 alkoxy or C1-C6 alkyl. In some embodiments, R^{A} is 5-10 membered heteroaryl substituted with C1-C6 alkoxy. In some embodiments, R^{A} is 5-10 membered heteroaryl substituted with C1-C6 alkyl. In some embodiments, R^{A} is 5-6 membered heteroaryl substituted with C1-C6 alkoxy. In some embodiments, R^{A} is 5-6 membered heteroaryl substituted with C1-C6 alkyl. In some embodiments, R^{A} is pyrrolyl, imidazolyl, oxazolyl, thiazolyl, pyridinyl, pyrimidinyl, or pyrazinyl; each optionally substituted with C1-C6 alkoxy or C1-C6 alkyl. In some embodiments, R^{A} is unsubstituted 5-6 membered heteroaryl.

In some embodiments, R^{A} is selected from the group consisting of:

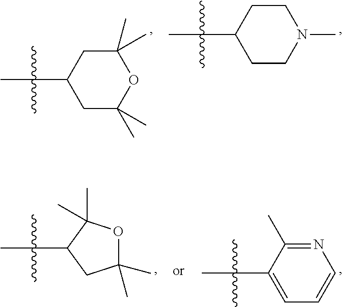

In some embodiments, R¹ is phenyl optionally substituted with —NR^{F}R^{G}. In some embodiments, R¹ is phenyl substituted with —NR^{F}R^{G}.

In some embodiments, R^{F} is C1-C6 alkyl. In some embodiments, R^{F} is methyl. In some embodiments, R^{F} is C3-C6 cycloalkyl. In some embodiments, R^{F} is hydrogen.

In some embodiments, R^{G} is C1-C6 alkyl. In some embodiments, R^{G} is methyl. In some embodiments, R^{G} is —C(═O)—C1-C6 alkyl. In some embodiments, R^{G} is —C(═O)CH₃. In some embodiments, R^{G} is —C(═O)—C3-C6 cycloalkyl. In some embodiments, R^{G} is hydrogen.

In some embodiments, R^{F} and R^{G} are the same. In some embodiments, R^{F} and R^{G} are different. In some embodiments, R^{F} and R^{G} are each hydrogen. In some embodiments, R^{F} and R^{G} are each methyl. In some embodiments, R^{F} is hydrogen and R^{G} is C1-C6 alkyl. In some embodiments, R^{F} is hydrogen and R^{G} is —C(═O)—C1-C6 alkyl.

In some embodiments, R¹ is -Q-R^{C}. In some embodiments, Q is C1-C6 alkylene. In some embodiments, Q is C1-C2 alkylene. In some embodiments, Q is methylene. In some embodiments, Q is NH. In some embodiments, Q is O.

In some embodiments, R^{C} is 4-10 membered heterocyclyl optionally substituted with —(C1-C6 alkylene)-NR^{D}R^{E}. In some embodiments, R^{C} is unsubstituted 4-10 membered heterocyclyl.

In some embodiments, R^{C} is 5-10 membered heteroaryl optionally substituted with —(C1-C6 alkylene)-NR^{D}R^{E}.

In some embodiments, R^{C} is phenyl optionally substituted with —(C1-C6 alkylene)-NR^{D}R^{E}.

In some embodiments, R¹ is

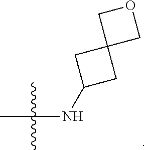 [SMILES: *NC1CC2(COC2)C1]

In some embodiments, R¹ is R^{G}

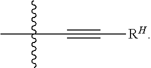

In some embodiments, R^{H} is 4-6 membered heterocyclyl optionally substituted with 1-2 independently selected C1-C6 alkyl. In some embodiments, R^{H} is 4-6 membered heterocyclyl substituted with 1-2 independently selected C1-C6 alkyl. In some embodiments, R^{H} is 4-6 membered heterocyclyl substituted with one C1-C6 alkyl. In some embodiments, R^{H} is 4-6 membered heterocyclyl substituted with methyl. In some embodiments, R^{H} is 4-6 membered heterocyclyl substituted with two independently selected C1-C6 alkyl. In some embodiments, R^{H} is 4-6 membered heterocyclyl substituted with two methyls. In some embodiments, R^{H} is oxetanyl, azetidinyl, pyrrolidinyl, pyrrolidinonyl, imidazolidinyl, piperidinyl, piperazinyl, or morpholinyl; each optionally substituted with 1-2 independently selected C1-C6 alkyl. In some embodiments, R^{H} is an unsubstituted 4-6 membered heterocyclyl.

In some embodiments, R² is C3-C6 cycloalkyl optionally substituted with —CO₂R^{B}. In some embodiments, R² is C3-C6 cycloalkyl substituted with —CO₂R^{B}. In some embodiments, R² is unsubstituted C3-C6 cycloalkyl.

In some embodiments, R^{B} is C1-C6 alkyl. In some embodiments, R^{B} is methyl. In some embodiments, R^{B} is hydrogen.

In some embodiments, R² is 5-10 membered heteroaryloxy. In some embodiments, R² is 5-6 membered heteroaryloxy. In some embodiments, R² is 9-10 membered heteroaryloxy.

In some embodiments, R² is —(C1-C6 alkylene)ₚ-5-10 membered heteroaryl optionally substituted with C1-C6 alkyl, cyano, or 4-6 membered heterocyclyl. In some embodiments, R² is —(C1-C6 alkylene)ₚ-5-10 membered heteroaryl substituted with C1-C6 alkyl, cyano, or 4-6 membered heterocyclyl. In some embodiments, R² is —(C1-C6 alkylene)ₚ-5-10 membered heteroaryl substituted with C1-C6 alkyl. In some embodiments, R² is —(C1-C6 alkylene)ₚ-5-10 membered heteroaryl substituted with methyl. In some embodiments, R² is —(C1-C6 alkylene)ₚ-5-10 membered heteroaryl substituted with cyano. In some embodiments, R² is —(C1-C6 alkylene)ₚ-5-10 membered heteroaryl substituted with 4-6 membered heterocyclyl. In some embodiments, R² is 5-10 membered heteroaryl substituted with 4-6 membered heterocyclyl. In some embodiments, R² is —(C1-C6 alkylene)ₚ-5-10 membered heteroaryl substituted with oxetanyl, azetidinyl, pyrrolidinyl, pyrrolidinonyl, imidazolidinyl, piperidinyl, piperazinyl, or morpholinyl. In some embodiments, R² is 5-10 membered heteroaryl substituted with oxetanyl, azetidinyl, pyrrolidinyl, pyrrolidinonyl, imidazolidinyl, piperidinyl, piperazinyl, or morpholinyl. In some embodiments, R² is 5-10 membered heteroaryl substituted with morpholinyl. In some embodiments, R² is unsubstituted —(C1-C6 alkylene)ₚ-5-10 membered heteroaryl.

In some embodiments, p is 1. In some embodiments, p is 0.

In some embodiments, R² is —(C1-C6 alkylene)ₜ-phenyl optionally substituted with cyano or —NR^{D}R^{E}. In some embodiments, R² is —(C1-C6 alkylene) t-phenyl substituted with cyano or —NR^{D}R^{E}. In some embodiments, R² is —(C1-C6 alkylene) t-phenyl substituted with —NR^{D}R^{E}In some embodiments, R^{D} is C1-C6 alkyl. In some embodiments, R^{D} is methyl. In some embodiments, R^{D} is C3-C6 cycloalkyl. In some embodiments, R^{D} is hydrogen. In some embodiments, R^{E} is C1-C6 alkyl. In some embodiments, R^{E} is methyl. In some embodiments, R^{E} is C3-C6 cycloalkyl. In some embodiments, R^{E} is hydrogen.

In some embodiments, t is 1. In some embodiments, t is 0.

In some embodiments, R² is 4-6 membered heterocyclyl optionally substituted with C1-C6 alkyl. In some embodiments, R² is 4-6 membered heterocyclyl substituted with C1-C6 alkyl. In some embodiments, R² is 4-6 membered heterocyclyl substituted with methyl. In some embodiments, R² is oxetanyl, azetidinyl, pyrrolidinyl, pyrrolidinonyl, imidazolidinyl, piperidinyl, piperazinyl, or morpholinyl; each optionally substituted with C1-C6 alkyl.

In some embodiments, R² is selected from the group consisting of:

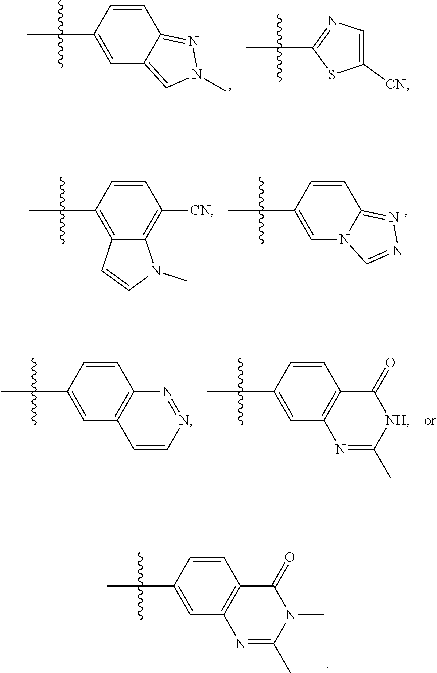

In some embodiments, R³ is C1-C3 alkyl. In some embodiments, R³ is methyl. In some embodiments, R^{C} is 4-10 membered heterocyclyl. In some embodiments, R^{C} is 4-6 membered heterocyclyl.

In some embodiments, R^{C} is 5-10 membered heteroaryl. In some embodiments, R^{C} is 5-6 membered heteroaryl.

In some embodiments, R^{C} is phenyl optionally substituted with —(C1-C6 alkylene)-NR^{D}R^{E}. In some embodiments, R^{C} is phenyl substituted with —(C1-C6 alkylene)-NR^{D}R^{E}. In some embodiments, R^{C} is phenyl substituted with —(C1-C2 alkylene)-NR^{D}R^{E}. In some embodiments, R^{C} is unsubstituted phenyl.

In some embodiments, R^{D} is C1-C6 alkyl. In some embodiments, R^{D} is methyl. In some embodiments, R^{D} is C3-C6 cycloalkyl. In some embodiments, R^{D} is hydrogen.

In some embodiments, R^{E} is C1-C6 alkyl. In some embodiments, R^{E} is methyl. In some embodiments, R^{E} is C3-C6 cycloalkyl. In some embodiments, R^{E} is hydrogen.

In some embodiments, m is 1. In some embodiments, m is 0.

In some embodiments, Ring A is 5-6 membered heteroaryl. In some embodiments, Ring A is thiazolyl, pyrazolyl, imidazolidinon-2-yl, pyridinyl, pyrimidinyl, pyridon-2-yl, pyrimidinonyl, or oxazolidinon-2-yl.

In some embodiments, Ring A is

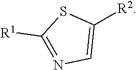

In some embodiments, Ring A is

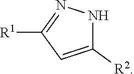

In some embodiments, Ring A is

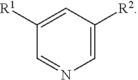

In some embodiments, Ring A is

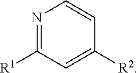

In some embodiments, Ring A is

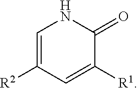

In some embodiments, Ring A is

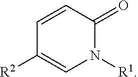

In some embodiments, Ring A is

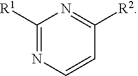

In some embodiments, Ring A is 5-6 membered heterocyclyl. In some embodiments, Ring A is oxazolidinone or pyrrolidinone.

In some embodiments, Ring A is

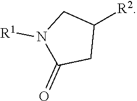

In some embodiments, Ring A is

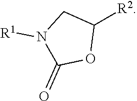

In some embodiments, the compound is a compound of Formula (III-1):

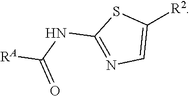

In some embodiments, the compound is a compound of Formula (III-2):

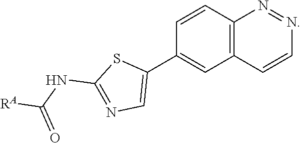

In some embodiments, the compound of Formula (III) is selected from a compound in Table 3, or a pharmaceutically acceptable salt thereof.

TABLE 3
Selected Compounds of Formula (III)

| Compound | |
|---|---|
| No. | Structure |
| 1 | 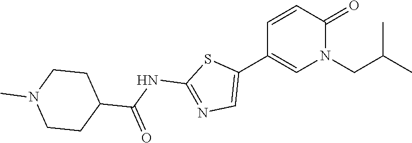 [SMILES: CC(C)Cn1cc(-c2cnc(NC(=O)C3CCN(C)CC3)s2)ccc1=O] |
| 2 | 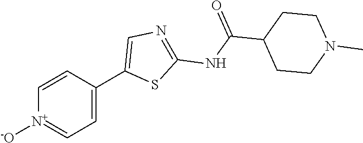 |
| 3 | 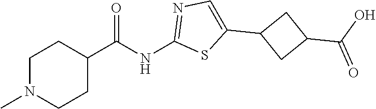 [SMILES: CN1CCC(C(=O)Nc2ncc(C3CC(C(=O)O)C3)s2)CC1] |
| 4 | 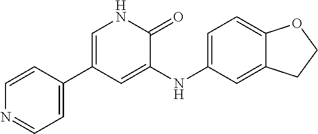 [SMILES: O=c1[nH]cc(-c2ccncc2)cc1Nc1ccc2c(c1)CCO2] |
| 5 | 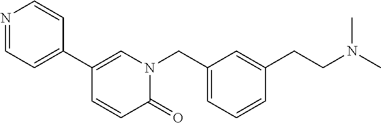 [SMILES: CN(C)CCc1cccc(Cn2cc(-c3ccncc3)ccc2=O)c1] |
| 6 | 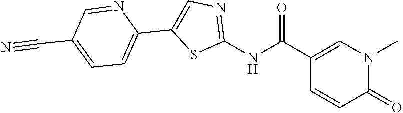 [SMILES: Cn1cc(C(=O)Nc2ncc(-c3ccc(C#N)cn3)s2)ccc1=O] |
| 7 | 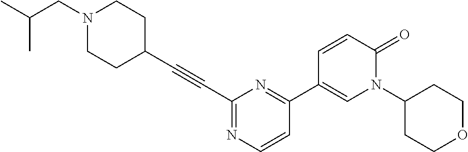 [SMILES: CC(C)CN1CCC(C#Cc2nccc(-c3ccc(=O)n(C4CCOCC4)c3)n2)CC1] |
| 8 | 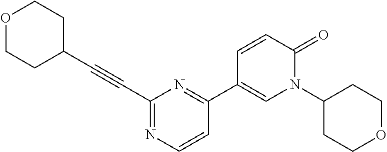 [SMILES: O=c1ccc(-c2ccnc(C#CC3CCOCC3)n2)cn1C1CCOCC1] |
| 9 | 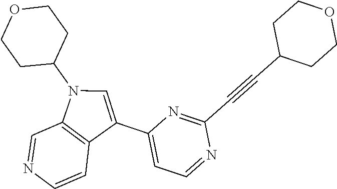 [SMILES: C(#CC1CCOCC1)c1nccc(-c2cn(C3CCOCC3)c3cnccc23)n1] |
| 10 | 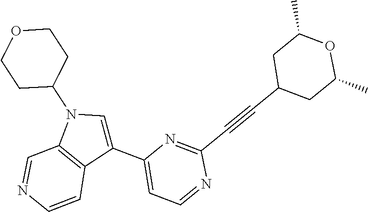 [SMILES: C[C@@H]1CC(C#Cc2nccc(-c3cn(C4CCOCC4)c4cnccc34)n2)C[C@H](C)O1] |
| 11 | 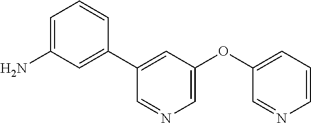 [SMILES: Nc1cccc(-c2cncc(Oc3cccnc3)c2)c1] |
| 12 | 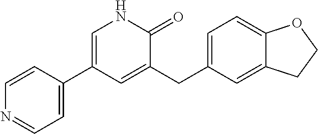 [SMILES: O=c1[nH]cc(-c2ccncc2)cc1Cc1ccc2c(c1)CCO2] |
| 13 | 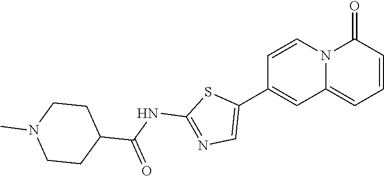 [SMILES: CN1CCC(C(=O)Nc2ncc(-c3ccn4c(=O)cccc4c3)s2)CC1] |
| 14 | 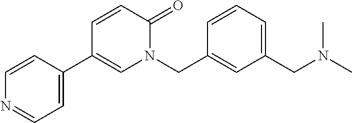 [SMILES: CN(C)Cc1cccc(Cn2cc(-c3ccncc3)ccc2=O)c1] |
| 15 | 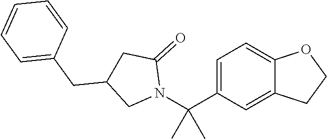 [SMILES: CC(C)(c1ccc2c(c1)CCO2)N1CC(Cc2ccccc2)CC1=O] |
| 16 | 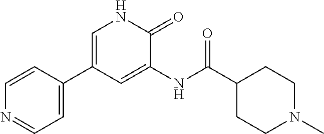 [SMILES: CN1CCC(C(=O)Nc2cc(-c3ccncc3)c[nH]c2=O)CC1] |
| 17 | 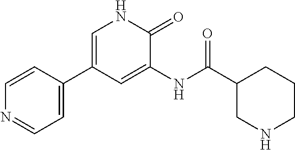 [SMILES: O=C(Nc1cc(-c2ccncc2)c[nH]c1=O)C1CCCNC1] |
| 18 | 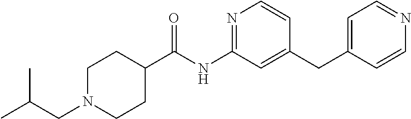 [SMILES: CC(C)CN1CCC(C(=O)Nc2cc(Cc3ccncc3)ccn2)CC1] |
| 19 | 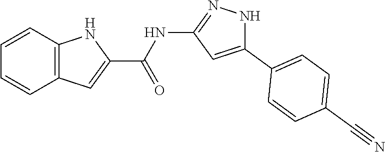 [SMILES: N#Cc1ccc(-c2cc(NC(=O)c3cc4ccccc4[nH]3)n[nH]2)cc1] |
| 20 | 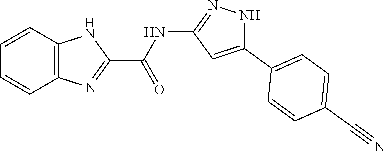 [SMILES: N#Cc1ccc(-c2cc(NC(=O)c3nc4ccccc4[nH]3)n[nH]2)cc1] |
| 21 | 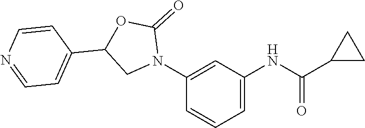 [SMILES: O=C(Nc1cccc(N2CC(c3ccncc3)OC2=O)c1)C1CC1] |
| 22 | 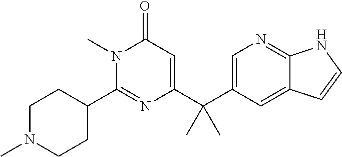 [SMILES: CN1CCC(c2nc(C(C)(C)c3cnc4[nH]ccc4c3)cc(=O)n2C)CC1] |
| 23 | 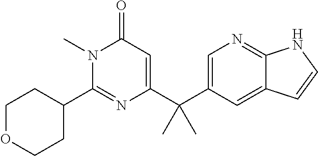 [SMILES: Cn1c(C2CCOCC2)nc(C(C)(C)c2cnc3[nH]ccc3c2)cc1=O] |
| 24 | 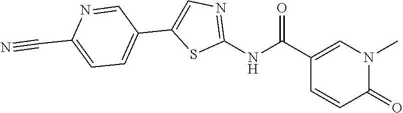 [SMILES: Cn1cc(C(=O)Nc2ncc(-c3ccc(C#N)nc3)s2)ccc1=O] |
| 25 | 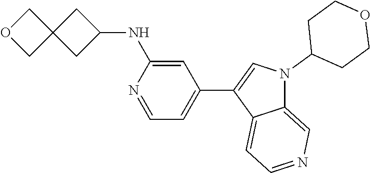 [SMILES: c1cc2c(-c3ccnc(NC4CC5(COC5)C4)c3)cn(C3CCOCC3)c2cn1] |
| 26 | 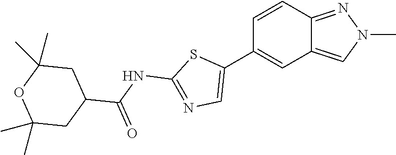 |
| 27 | 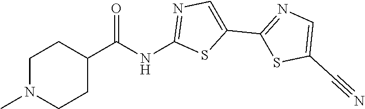 [SMILES: CN1CCC(C(=O)Nc2ncc(-c3ncc(C#N)s3)s2)CC1] |
| 28 | 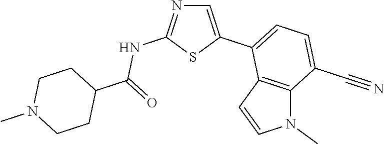 [SMILES: CN1CCC(C(=O)Nc2ncc(-c3ccc(C#N)c4c3ccn4C)s2)CC1] |
| 29 | 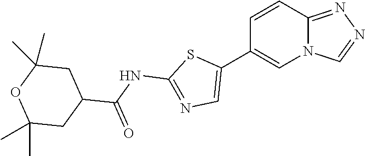 [SMILES: CC1(C)CC(C(=O)Nc2ncc(-c3ccc4nncn4c3)s2)CC(C)(C)O1] |
| 30 | 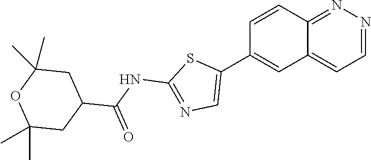 [SMILES: CC1(C)CC(C(=O)Nc2ncc(-c3ccc4nnccc4c3)s2)CC(C)(C)O1] |
| 31 | 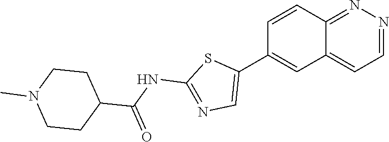 [SMILES: CN1CCC(C(=O)Nc2ncc(-c3ccc4nnccc4c3)s2)CC1] |
| 32 | 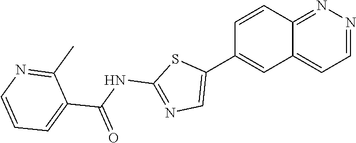 [SMILES: Cc1ncccc1C(=O)Nc1ncc(-c2ccc3nnccc3c2)s1] |
| 33 | 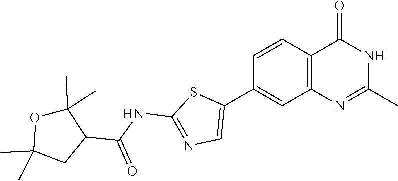 |
| 34 | 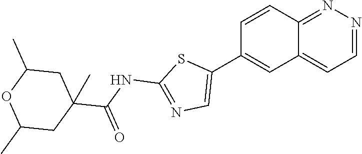 [SMILES: CC1CC(C)(C(=O)Nc2ncc(-c3ccc4nnccc4c3)s2)CC(C)O1] |
| 35 | 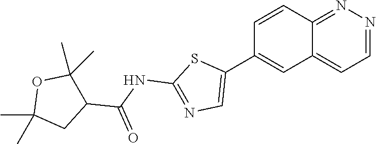 [SMILES: CC1(C)CC(C(=O)Nc2ncc(-c3ccc4nnccc4c3)s2)C(C)(C)O1] |
| 36 | 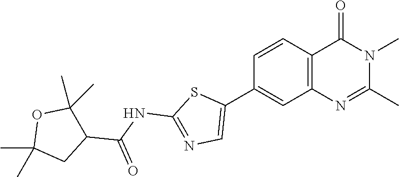 |
| 37 | 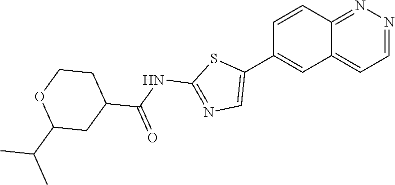 [SMILES: CC(C)C1CC(C(=O)Nc2ncc(-c3ccc4nnccc4c3)s2)CCO1] |
| 38 | 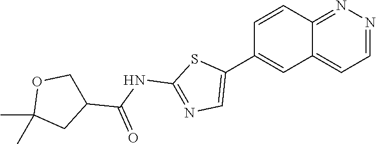 [SMILES: CC1(C)CC(C(=O)Nc2ncc(-c3ccc4nnccc4c3)s2)CO1] |

### Pharmaceutical Compositions and Administration

### General

In some embodiments, the compounds described herein (e.g., compounds of Formula (I), (II), or (III), and pharmaceutically acceptable salts of any of the foregoing), are administered as a pharmaceutical composition that includes the chemical compound and one or more pharmaceutically acceptable excipients. In some embodiments, the compound or a pharmaceutically acceptable salt thereof is a compound of Formula (I), or a pharmaceutically acceptable salt thereof. In some embodiments, the compound or a pharmaceutically acceptable salt thereof is a compound of Formula (II), or a pharmaceutically acceptable salt thereof. In some embodiments, the compound or a pharmaceutically acceptable salt thereof is a compound of Formula (III), or a pharmaceutically acceptable salt thereof.

In some embodiments, the compounds can be administered in combination with one or more conventional pharmaceutical excipients as described herein. Dosage forms or compositions containing a chemical entity as described herein in the range of 0.005% to 100% with the balance made up from one or more pharmaceutically acceptable excipients may be prepared. The contemplated compositions may contain 0.001%-100% of a compound (or pharmaceutically acceptable salt thereof) provided herein, for example, from 0.1-95%, 75-85%, or 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

### Routes of Administration and Composition Components

In some embodiments, the compounds described herein or a pharmaceutical composition thereof can be administered to subject in need thereof by any accepted route of administration. Acceptable routes of administration include, but are not limited to, buccal, epidural, intracerebral, intradural, intramedullary, intrameningeal, intramuscular, intraspinal, intravascular, intravenous, nasal, oral, parenteral, peridural, respiratory (inhalation), subcutaneous, sublingual, submucosal, topical, transdermal, and transmucosal. In certain embodiments, a preferred route of administration is parenteral. In certain embodiments, a preferred route of administration is oral.

Compositions can be formulated for parenteral administration, e.g., formulated for injection via the intravenous, intramuscular, or sub-cutaneous routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the compounds is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In some embodiments, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds (i.e., excipients) include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

### Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 1 mg/kg; from about 0.1 mg/kg to about 0.5 mg/kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Methods of Treatment

### Indications

This disclosure provides compounds of Formula (I), (II), or (III), and pharmaceutically acceptable salts of any of the foregoing, that inhibit Dual specificity tyrosine-phosphorylation-regulated kinase 1A (DYRK1A). These compounds are useful for treating neurological disorders, e.g., DYRK1A-associated neurological disorders. In some embodiments, the compound or a pharmaceutically acceptable salt thereof is a compound of Formula (I), or a pharmaceutically acceptable salt thereof. In some embodiments, the compound or a pharmaceutically acceptable salt thereof is a compound of Formula (II), or a pharmaceutically acceptable salt thereof. In some embodiments, the compound or a pharmaceutically acceptable salt thereof is a compound of Formula (III), or a pharmaceutically acceptable salt thereof.

“Neurological disorder” refers to any disease or disorder of the nervous system and/or visual system. “Neurological disease” or “neurological disorder” are used interchangeably herein, and include diseases or disorders that involve the central nervous system (CNS; e.g., brain, brainstem and cerebellum), the peripheral nervous system (PNS; including cranial nerves), and the autonomic nervous system (parts of which are located in both the CNS and PNS), including both structural and/or functional diseases and disorders (e.g., neurological syndrome).

Examples of neurological disorders include, but are not limited to, headache, stupor and coma, dementia, seizure, sleep disorders, trauma, infections, neoplasms, neuroopthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological disorder. The following is a list of several neurological disorders, symptoms, signs and syndromes that can be treated using compositions and methods according to the present invention: acquired epileptiform aphasia; acute disseminated encephalomyelitis; adrenoleukodystrophy; agenesis of the corpus callosum; agnosia: Aicardi syndrome; Alexander disease; Alpers' disease; alternating hemiplegia; vascular dementia; amyotrophic lateral sclerosis; anencephaly; Angelman syndrome; angiomatosis; anoxia; aphasia; apraxia; arachnoid cysts; arachnoiditis; Anronl-Chiari malformation; arteriovenous malformation; Asperger syndrome; ataxia telegiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm: Bloch Sulzberger syndrome; brachial plexus injury; brain abscess; brain injury; Brown-Sequard syndrome; Cana van disease; carpal tunnel syndrome; causalgia; central pain syndrome; central pontine myelinolysis: cephalic disorder; cerebral aneurysm; cerebral arteriosclerosis: cerebral atrophy; cerebral gigantism; cerebral palsy; Charcot-Marie-Tooth disease; Chiari malformation; chorea; chronic inflammatory demyelinating polyneuropathy; chronic pain; chronic regional pain syndrome; Coffin Lowry syndrome; coma, including persistent vegetative state; congenital facial diplegia; corticobasal degeneration: cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; DandyWalker syndrome; Dawson disease; De Moisier's syndrome; Dejerine-Klumke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; fronto-temporal dementia; Gaucher's disease; Gerstmann's syndrome; giant cell arteritis; giant cell inclusion disease; globoid cell leukodystrophy; Guillain-Barre syndrome; HTLV-1-associated myelopathy; Hallervorden-Spatz disease; head injury; headache; hemifacial spasm; hereditary spastic paraplegia: heredopathia atactic a polyneuritiformis; herpes zoster oticus; herpes zoster; Hirayama syndrome; HIV associated dementia and neuropathy (also neurological manifestations of AIDS); holoprosencephaly; Huntington's disease and other polyglutamine repeat diseases; hydranencephaly: hydrocephalus; hypercortisolism; hypoxia; immune-mediated encephalomyelitis; inclusion body myositis; incontinentia pigmenti; infantile phytanic acid storage disease; infantile spasms; inflammatory myopathy; intracranial cyst; intracranial hypertension; Joubert syndrome; Kearns-Sayre syndrome; Kennedy disease Kinsboume syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gustaut syndrome; Lesch-Nyhan syndrome; leukodystrophy; Lewy body dementia; Lissencephaly; locked-in syndrome; Lou Gehrig's disease (i.e., motor neuron disease or amyotrophic lateral sclerosis); lumbar disc disease; Lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neuron disease; Moyamoya disease; mucopolysaccharidoses; milti-infarct dementia; multifocal motor neuropathy; multiple sclerosis and other demyelinating disorders; multiple system atrophy with postural hypotension; p muscular dystrophy; myasthenia gravis; myelmociastic diffuse sclerosis; myoclonic encephalopathy of infants; myoclonus; myopathy; myotonia congenital; narcolepsy: neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Neurodegenerative disease or disorder (Parkinson's disease, Huntington's disease, Alzheimer's disease, amyotrophic lateral sclerosis (ALS), dementia, multiple sclerosis and other diseases and disorders associated with neuronal cell death); paramyotonia congenital; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocalleukoencephalopathy; progressive sclerosing poliodystrophy; progressive supranuclear palsy; Ramsay-Hunt syndrome (types I and II); Rasmussen's encephalitis: reflex sympathetic dystrophy syndrome; Refsum disease; repetitive motion disorders, repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia: shaken baby syndrome: shingles: Shy-Drager syndrome; Sjogren's syndrome; Soto's syndrome; spasticity; spina bifida; spinal cord injury; spinal muscular atrophy; Stiff-Person syndrome; stroke; Sturge-Weber syndrome; subacute sclerosing panencephalitis; subcortical arteriosclerotic encephalopathy; Sydenham chorea; syncope; syringomyelia; tardive dyskinesia; Tay-Sachs disease; temporal arteritis; tethered spinal cord syndrome; Thomsen disease; thoracic outlet syndrome; Tic Douloureux; Todd's paralysis; Tourette syndrome; transient ischemic attack; transmissible spongiform encephalopathies; transverse myelitis; traumatic brain injury; tremor, trigeminal neuralgia; tropical spastic paraparesis: tuberous sclerosis; vascular dementia (multi-infarct dementia); vasculitis including temporal arteritis; Von Hippel-Liodau disease; Wallenberg's syndrome; Werdnig-Hoffman disease; West syndrome; Williams syndrome; Wildoris disease; and Zellweger syndrome.

In some embodiments, the neurological disease or neurological disorder is Alzheimer's disease, Down syndrome, Alzheimer's disease associated with Down syndrome, Parkinson's disease, ALS, dementia, Huntington's disease, multiple sclerosis, proximal lateral sclerosis, stroke, stroke, or mild cognitive impairment.

In some embodiments, the dementia may be Alzheimer's dementia, cerebrovascular dementia, dementia due to head injury, multi-infarct dementia, mixed or alcoholic dementia of Alzheimer's disease and multi-infarct dementia.

The ability of test compounds to act as inhibitors of DYRK1A may be demonstrated by assays known in the art. The activity of the compounds and compositions provided herein as DYRK1A inhibitors can be assayed in vitro, in vivo, or in a cell line. In vitro assays include assays that determine inhibition of the kinase. Alternate in vitro assays quantitate the ability of the inhibitor to bind to the protein kinase and can be measured either by radio labelling the compound prior to binding, isolating the compound/kinase complex and determining the amount of radio label bound, or by running a competition experiment where new compounds are incubated with the kinase bound to known radio ligands.

Potency of a DYRK1A inhibitor as provided herein can be determined by EC₅₀ or IC₅₀ values. A compound with a lower EC₅₀ or IC₅₀ value, as determined under substantially similar conditions, is a more potent inhibitor relative to a compound with a higher EC₅₀ or IC₅₀ value. In some embodiments, the substantially similar conditions comprise determining a DYRK1A-dependent phosphorylation level, in vitro or in vivo (e.g., in neural cells, such as neurons, astrocytes, oligodendrocytes, microglia, ependymal cells, Schwann cells, and satellite cells, expressing a wild type DYRK1A, a mutant DYRK1A, or a fragment of any thereof).

Potency of a DYRK1A inhibitor as provided herein can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining a DYRK1A-dependent phosphorylation level, in vitro or in vivo (e.g., in neural cells, such as neurons, astrocytes, oligodendrocytes, microglia, ependymal cells, Schwann cells, and satellite cells, expressing a wild type DYRK1A, a mutant DYRK1A, or a fragment of any thereof).

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease or disorder to be treated and/or prevented.

In some embodiments, the subject has been identified or diagnosed as having a neurological disorder with a dysregulation of a DYRK1A gene, a DYRK1A protein, or expression or activity, or level of any of the same (a DYRK1A-associated neurological disorder) (e.g., as determined using a regulatory agency-approved, e.g., FDA-approved, assay or kit). The subject can be a subject that is positive for a dysregulation of a DYRK1A gene, a DYRK1A protein, or expression or activity, or level of any of the same (e.g., identified as positive using a regulatory agency-approved, e.g., FDA-approved, assay or kit). In some embodiments, the subject is suspected of having a DYRK1A-associated neurological disorder. In some embodiments, the subject has a clinical record indicating that the subject has a neurological disorder that has a dysregulation of a DYRK1A gene, a DYRK1A protein, or expression or activity, or level of any of the same (and optionally the clinical record indicates that the subject should be treated with any of the compositions provided herein).

In certain embodiments, compounds of Formula (I), (II), or (III), or pharmaceutically acceptable salts of any of the foregoing, are useful for preventing neurological disorders as defined herein (for example, Alzheimer's disease). The term “preventing” as used herein means to delay the onset, recurrence or spread, in whole or in part, of the disease or condition as described herein, or a symptom thereof.

The term “DYRK1A-associated neurological disorder” as used herein refers disorders associated with or having a dysregulation of a DYRK1A gene, a DYRK1A protein, or the expression or activity or level of any (e.g., one or more) of the same (e.g., any of the types of dysregulation of a DYRK1A gene, or a DYRK1A protein, or the expression or activity or level of any of the same described herein). Non-limiting examples of a DYRK1A-associated disease or disorder include, for example, Down Syndrome, Alzheimer's disease, and Alzheimer's disease associated with Down Syndrome.

The phrase “dysregulation of a DYRK1A gene, a DYRK1A protein, or the expression or activity or level of any of the same” refers to a gene duplication (or multiplication) that results in an increased level of DYRK1A in a cell, or a mutation in a regulatory sequence (e.g., a promoter and/or enhancer) that results in an increased level of DYRK1A in a cell), or increased expression (e.g., increased levels) of a wild type DYRK1A in a mammalian cell due to aberrant cell signaling and/or dysregulated autocrine/paracrine signaling (e.g., as compared to a control cell lacking the aberrant signaling).

Some embodiments provide a method for treating a neurological disorder in a subject in need thereof, the method comprises administering to the subject a therapeutically effective amount of a compound of Formula (I), (II), or (III), or a pharmaceutically acceptable salt of any of the foregoing, or a pharmaceutical composition comprising a compound of Formula (I), (II), or (III), or a pharmaceutically acceptable salt of any of the foregoing.

In some embodiments, the method for treating a neurological disorder in a subject in need thereof, comprises (a) determining that the neurological disorder is associated with a dysregulation of a DYRK1A gene, a DYRK1A protein, or expression or activity or level of any of the same; and (b) administering to the subject a therapeutically effective amount of a compound of Formula (I), (II), or (III), or a pharmaceutically acceptable salt of any of the foregoing, or a pharmaceutical composition comprising a compound of Formula (I), (II), or (III), or a pharmaceutically acceptable salt of any of the foregoing.

Some embodiments provide a method of treating a DYRK1A-associated neurological disorder in a subject, the method comprises administering to a subject identified or diagnosed as having a DYRK1A-associated neurological disorder a therapeutically effective amount of a compound of Formula (I), (II), or (III), or a pharmaceutically acceptable salt of any of the foregoing, or a pharmaceutical composition comprising a compound of Formula (I), (II), or (III), or a pharmaceutically acceptable salt of any of the foregoing.

In some embodiments, the method of treating a DYRK1A-associated neurological disorder in a subject, comprises:
(a) determining that the neurological disorder in the subject is a DYRK1A-associated neurological disorder; and
(b) administering to the subject a therapeutically effective amount of a compound of Formula (I), (II), or (III), or a pharmaceutically acceptable salt of any of the foregoing, or a pharmaceutical composition comprising a compound of Formula (I), (II), or (III), or a pharmaceutically acceptable salt of any of the foregoing.

Some embodiments provide a method of treating a subject, the method comprises administering a therapeutically effective amount of a compound of Formula (I), (II), or (III), or a pharmaceutically acceptable salt of any of the foregoing, or a pharmaceutical composition comprising a compound of Formula (I), (II), or (III), or a pharmaceutically acceptable salt of any of the foregoing, to a subject having a clinical record that indicates that the subject has a dysregulation of a DYRK1A gene, DYRK1A protein, or expression or activity or level of any of the same.

In some embodiments, the method comprises the step of determining that the neurological disorder in the subject is a DYRK1A-associated neurological disorder and includes performing an assay to detect dysregulation in a DYRK1A gene, a DYRK1A protein, or expression or activity or level of any of the same in a sample from the subject.

Some embodiments provide a method for treating a neurological disorder in a subject in need thereof, the method comprising (a) determining that the neurological disorder is associated with Down Syndrome; and (b) administering to the subject a therapeutically effective amount of a compound of Formula (I), (II), or (III), or a pharmaceutically acceptable salt of any of the foregoing, or a pharmaceutical composition comprising a compound of Formula (I), (II), or (III), or a pharmaceutically acceptable salt of any of the foregoing.

In some embodiments, the step of determining that the neurological disorder in the subject is associated with Down Syndrome includes performing an assay on a sample from the subject.

In some embodiments, the method further comprises obtaining a sample from the subject. In some embodiments, the sample is a blood sample. In some embodiments, the sample is a sample of cerebrospinal fluid (CSF).

In some embodiments, the assay is selected from the group consisting of sequencing, immunohistochemistry, enzyme-linked immunosorbent assay, and fluorescence in situ hybridization (FISH).

In some embodiments, the FISH is break apart FISH analysis. In some embodiments, the sequencing is pyrosequencing or next generation sequencing.

In some embodiments, the DYRK1A-associated neurological disorder is selected from the group consisting of Down Syndrome, Alzheimer's disease, and Alzheimer's disease associated with Down syndrome. In some embodiments, the DYRK1A-associated neurological disorder is Alzheimer's disease associated with Down syndrome.

In some embodiments, the method further comprises administering to the subject an additional therapy or therapeutic agent as described herein.

Some embodiments provide a method for modulating DYRK1A in a mammalian cell, the method comprises contacting the mammalian cell with a therapeutically effective amount of a compound of a Formula (I), (II), or (III), or a pharmaceutically acceptable salt of any of the foregoing.

In some embodiments, the contacting occurs in vivo. In some embodiments, the contacting occurs in vitro. In some embodiments, the mammalian cell is a mammalian neural cell. In some embodiments, the mammalian neural cell is a mammalian DYRK1A-associated neural cell. In some embodiments, the cell has a dysregulation of a DYRK1A gene, a DYRK1A protein, or expression or activity or level of any of the same. In some embodiments, the cell has a chromosomal abnormality associated with Down Syndrome.

Exemplary Sequence of Human Dual specificity tyrosine-phosphorylation-regulated kinase 1A (UniProtKB entry Q13627) (SEQ ID NO: 1)

MHTGGETSAC KPSSVRLAPS FSFHAAGLQM AGQMPHSHQY
SDRRQPNISD QQVSALSYSD QIQQPLTNQV MPDIVMLQRR
MPQTFRDPAT APLRKLSVDL IKTYKHINEV YYAKKKRRHQ
QGQGDDSSHK KERKVYNDGY DDDNYDYIVK NGEKWMDRYE
IDSLIGKGSF GQVVKAYDRV EQEWVAIKII KNKKAFLNQA
QIEVRLLELM NKHDTEMKYY IVHLKRHFMF RNHLCLVFEM
LSYNLYDLLR NTNFRGVSLN LTRKFAQQMC TALLFLATPE
LSIIHCDLKP ENILLCNPKR SAIKIVDFGS SCQLGQRIYQ
YIQSRFYRSP EVLLGMPYDL AIDMWSLGCI LVEMHTGEPL
FSGANEVDQM NKIVEVLGIP PAHILDQAPK ARKFFEKLPD
GTWNLKKTKD GKREYKPPGT RKLHNILGVE TGGPGGRRAG
ESGHTVADYL KFKDLILRML DYDPKTRIQP YYALQHSFFK
KTADEGTNTS NSVSTSPAME QSQSSGTTSS TSSSSGGSSG
TSNSGRARSD PTHQHRHSGG HFTAAVQAMD CETHSPQVRQ
QFPAPLGWSG TEAPTQVTVE THPVQETTFH VAPQQNALHH
HHGNSSHHHH HHHHHHHHHG QQALGNRTRP RVYNSPTNSS
STQDSMEVGH SHHSMTSLSS STTSSSTSSS STGNQGNQAY
QNRPVAANTL DFGQNGAMDV NLTVYSNPRQ ETGIAGHPTY
QFSANTGPAH YMTEGHLTMR QGADREESPM TGVCVQQSPV
ASS

In some embodiments, compounds of Formula (I), (II), or (III), or a pharmaceutically acceptable salt of any of the foregoing, are useful for treating a neurological disorder that has been identified as being associated with dysregulation of DYRK1A. Accordingly, provided herein are methods for treating a subject diagnosed with (or identified as having) a neurological disorder that include administering to the subject a therapeutically effective amount of a compound of Formula (I), (II), or (III), or a pharmaceutically acceptable salt of any of the foregoing.

Also provided herein are methods for treating a subject identified or diagnosed as having a DYRK1A-associated neurological disorder that include administering to the subject a therapeutically effective amount of a compound of Formula (I), (II), or (III), or a pharmaceutically acceptable salt of any of the foregoing, or a pharmaceutical composition thereof. In some embodiments, the subject that has been identified or diagnosed as having a DYRK1A-associated neurological disorder through the use of a regulatory agency-approved, e.g., FDA-approved test or assay for identifying dysregulation of a DYRK1A gene, a DYRK1A protein, or expression or activity or level of any of the same, in a subject or a biological sample (e.g., blood and/or CSF) from the subject or by performing any of the non-limiting examples of assays described herein. In some embodiments, the test or assay is provided as a kit. In some embodiments, the neurological disorder is a DYRK1A-associated neurological disorder.

The term “regulatory agency” refers to a country's agency for the approval of the medical use of pharmaceutical agents with the country. For example, a non-limiting example of a regulatory agency is the U.S. Food and Drug Administration (FDA).

Also provided is a method for inhibiting DYRK1A activity in a cell, comprising contacting the cell with a compound of Formula (I), (II), or (III), or a pharmaceutically acceptable salt of any of the foregoing. In some embodiments, the contacting is in vitro. In some embodiments, the contacting is in vivo. In some embodiments, the contacting is in vivo, wherein the method comprises administering a therapeutically effective amount of a compound of Formula (I), (II), or (III), or a pharmaceutically acceptable salt of any of the foregoing, to a subject having a cell having aberrant DYRK1A activity. In some embodiments, the cell is a neural cell. In some embodiments, the neural cell is a DYRK1A-associated neural cell.

As used herein, the term “contacting” refers to the bringing together of indicated moieties in an in vitro system or an in vivo system. For example, “contacting” a DYRK1A protein with a compound provided herein includes the administration of a compound provided herein to an individual or subject, such as a human, having a DYRK1A protein, as well as, for example, introducing a compound provided herein into a sample containing a cellular or purified preparation containing the DYRK1A protein.

The phrase “therapeutically effective amount” means an amount of compound that, when administered to a subject in need of such treatment, is sufficient to (i) treat a DYRK1A protein-associated disease or disorder, (ii) attenuate, ameliorate, or eliminate one or more symptoms of the particular disease, condition, or disorder, or (iii) delay the onset of one or more symptoms of the particular disease, condition, or disorder described herein. The amount of a compound of Formula (I), (II), or (III), or a pharmaceutically acceptable salt of any of the foregoing, that will correspond to such an amount will vary depending upon factors such as the particular compound, disease condition and its severity, the identity (e.g., weight) of the subject in need of treatment, but can nevertheless be routinely determined by one skilled in the art.

When employed as pharmaceuticals, the compounds of Formula (I), (II), and (III), including pharmaceutically acceptable salts of any of the foregoing, can be administered in the form of pharmaceutical compositions as described herein.

### Combinations

In some embodiments, of any of the methods described herein, the compound of Formula (I), (II), or (III), or a pharmaceutically acceptable salt of any of the foregoing, is administered in combination with a therapeutically effective amount of at least one additional therapeutic agent selected from one or more additional therapies or therapeutic agents.

In some embodiments, the methods described herein further comprise administering one or more additional therapies selected from typical antipsychotics, atypical antipsychotics, antidepressants, electroconvulsive therapy, transcranial magnetic stimulation, benzodiazepines, mood stabilizers, cholinesterase inhibitors, memantine, NSAIDs, analgesics, anxiolytics, gabapentin and pregabalin.

In some embodiments, the methods described herein further comprise providing cognitive behavior therapy to the subject.

In some embodiments, the one or more additional therapies is a standard of care treatment for neuropathic pain. In some embodiments, the one or more additional therapies is a standard of care treatment for Alzheimer's disease. In some embodiments, the one or more additional therapies is a standard of care treatment for Alzheimer's disease associated with Down Syndrome.

In some embodiments, the one or more additional therapies is a typical antipsychotic. Representative typical antipsychotics include, but are not limited to chlorpromazine, chlorprothixene, levomepromazine, mesoridazine, periciazine, promazine, loxapine, molindone, perphenazine, thiothixene, droperidol, flupentixol, fluphenazine, haloperidol, pimozide, prochlorperazine, thioproperazine, trifluoperazine, and zuclopenthixol.

In some embodiments, the one or more additional therapies is an atypical antipsychotic. Representative atypical antipsychotics include, but are not limited to aripiprazole, risperidone, olanzapine, quetiapine, asenapine, paliperidone, ziprasidone, or lurasidone.

In some embodiments, the one or more additional therapies is an antidepressant. In some embodiments, the antidepressant is an atypical antidepressant, a selective serotonin reuptake inhibitor, a selective serotonin and norepinephrine reuptake inhibitor, a monoamine oxidase inhibitor, a selective norepinephrine reuptake inhibitor, or a tricyclic antidepressant.

In some embodiments, the compound of Formula (I), (II), or (III), or a pharmaceutically acceptable salt of any of the foregoing, and the one or more additional therapies are administered as separate dosages sequentially in any order. In some embodiments, the compound of Formula (I), (II), or (III), or a pharmaceutically acceptable salt of any of the foregoing, and the one or more additional therapies are administered as a single dosage form.

In some embodiments, the antidepressant is an atypical antidepressant. Representative atypical antidepressants include, but are not limited to mirtazapine, mianserin, bupropion, trazodone, nefazodone, tianeptine, opipramol, agomelatine, vilazodone, and vortioxetine.

In some embodiments, the antidepressant is a selective serotonin reuptake inhibitor. Representative selective serotonin reuptake inhibitors include, but are not limited to citalopram, escitalopram, fluoxetine, fluvoxamine, paroxetine, and sertraline.

In some embodiments, the antidepressant is a selective serotonin and norepinephrine reuptake inhibitor. Representative selective serotonin and norepinephrine reuptake inhibitors include, but are not limited to atomoxetine, desvenlafaxine, duloxetine, levomilnacipran, milnacipran, sibutramine, tramadol, and venlafaxine.

In some embodiments, the antidepressant is a monoamine oxidase inhibitor. Representative monoamine oxidase inhibitors include, but are not limited to moclobemide, rasagiline, selegiline, or safinamide.

In some embodiments, the antidepressant is a selective norepinephrine reuptake inhibitor. Representative selective norepinephrine reuptake inhibitors include, but are not limited to reboxetine.

In some embodiments, the antidepressant is a tricyclic antidepressant. Representative tricyclic antidepressants include, but are not limited to amineptine, amitriptyline, amoxapine, butriptyline, clomipramine, desipramine, dibenzepin, dosulepin, doxepin, imipramine, iprindole, lofepramine, maprotiline, norclomipramine, northiaden, nortriptyline, pipramol, protriptyline, tianeptine, and trimipramine.

In some embodiments, the one or more additional therapies is a benzodiazepine. Representative benzodiazepines include, but are not limited to alprazolam, bromazepam, chlordiazepoxide, clonazepam, clorazepate, diazepam, flurazepam, lorazepam, oxazepam, temazepam, or triazolam.

In some embodiments, the one or more additional therapies is a mood stabilizer. Representative mood stabilizers include, but are not limited to lithium, valproic acid, lamotrigine, or carbamazepine. In some embodiments, the one or more additional therapies is electroconvulsive therapy or transcranial magnetic stimulation.

In some embodiments, the one or more additional therapies is sertraline. In some embodiments, the one or more additional therapies is venlafaxine.

In some embodiments, the one or more additional therapies is a cholinesterase inhibitor. Representative cholinesterase inhibitors include, but are not limited to donepezil, galantamine, and rivastigmine.

In some embodiments, the one or more additional therapies is memantine.

In some embodiments, the one or more additional therapies is an NSAID. Representative NSAIDs include, but are not limited to clonixin, licofelone, salicylates (such as aspirin and diflunisal), propionic acid derivative (such as ibuprofen, dexibuprofen, naproxen, fenoprofen, ketoprofen, dexketoprofen, flurbiprofen, and oxaprozin), acetic acid derivatives (such as indomethacin, tolmetin, sulindac, etodolac, ketorolac, diclofenac, aceclofenac, and bromfenac), and COX-2 inhibitors (such as celecoxib).

In some embodiments, the one or more additional therapies is an analgesic. Representative analgesics include, but are not limited to nefopam, flupiritine, ziconotide, acetaminophen, and opioids (such as morphine, oxycodone, methadone, codeine, fentanyl, hydrocodone, and tramadol).

In some embodiments, the one or more additional therapies is an anxiolytic. Representative anxiolytics include, but are not limited to alnespirone, adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, and zolazepam.

In some embodiments, the one or more additional therapies is gabapentin or pregabalin.

In some embodiments, the one or more additional therapies is one additional therapy. In some embodiments, the one or more additional therapies is two, three, or four additional therapies. Some embodiments provide a method of treating a neurological disorder, comprising administering a compound of Formula (I), (II), or (III), or a pharmaceutically acceptable salt of any of the foregoing, and one or more additional therapies selected from typical antipsychotics, atypical antipsychotics, antidepressants, electroconvulsive therapy, transcranial magnetic stimulation, benzodiazepines, mood stabilizers, cholinesterase inhibitors, memantine, NSAIDs, analgesics, anxiolytics, gabapentin and pregabalin, to a subject in need thereof.

In some embodiments, the subject was being administered the one or more additional therapies prior to initiation of treatment with a compound of Formula (I), (II), or (III), or a pharmaceutically acceptable salt of any of the foregoing. In some embodiments, the subject was being administered the one or more additional therapies prior to initiation of treatment with a compound of Formula (I), (II), or (III), or a pharmaceutically acceptable salt of any of the foregoing, but after treatment with a compound of Formula (I), (II), or (III), or a pharmaceutically acceptable salt of any of the foregoing, for a period of time, the subject is no longer administered the one or more additional therapies. In some embodiments, of this paragraph, the period of time is about 1 month to about 1 year, for example, about 1 month to about 5 months, about 3 months to about 8 months, about 7 months to about 1 year, about 1 month, about 2 months, about 3 months, about 4 months, about 5 months, about 6 months, about 7 months, about 8 months, about 9 months, about 10 months, about 11 months, about 12 months, or any value in between. In some embodiments, the amount of the one or more additional therapies is decreased during the period of time, to zero at the end of the period of time.

In some embodiments, the subject has previously been administered one or more additional therapies selected from typical antipsychotics, atypical antipsychotics, antidepressants, electroconvulsive therapy, transcranial magnetic stimulation, benzodiazepines, mood stabilizers, cholinesterase inhibitors, memantine, NSAIDs, analgesics, anxiolytics, gabapentin, and pregabalin; wherein the subject was not responsive to the previous one or more therapies.

In some embodiments, the subject has previously been administered a standard of care treatment for neuropathic pain and the subject was not responsive to the previous therapy. In some embodiments, the subject has previously been administered a standard of care treatment for Alzheimer's disease and the subject was not responsive to the previous therapy. In some embodiments, the subject has previously been administered a standard of care treatment for Alzheimer's disease associated with Down Syndrome and the subject was not responsive to the previous therapy.

In some embodiments, the subject has previously been administered one or more additional therapies selected from typical antipsychotics, atypical antipsychotics, antidepressants, electroconvulsive therapy, transcranial magnetic stimulation, benzodiazepines, mood stabilizers, cholinesterase inhibitors, memantine, NSAIDs, analgesics, anxiolytics, gabapentin, and pregabalin, and was not responsive to the previous therapy.

In some embodiments, the subject has previously been administered one or more typical antipsychotics such as chlorpromazine, chlorprothixene, levomepromazine, mesoridazine, periciazine, promazine, loxapine, molindone, perphenazine, thiothixene, droperidol, flupentixol, fluphenazine, haloperidol, pimozide, prochlorperazine, thioproperazine, trifluoperazine, and zuclopenthixol, and was not responsive to the previous therapy.

In some embodiments, the subject has previously been administered one or more atypical antipsychotics, such as aripiprazole, risperidone, olanzapine, quetiapine, asenapine, paliperidone, ziprasidone, or lurasidone, and was not responsive to the previous therapy.

In some embodiments, the subject has previously been administered one or more antidepressants and was not responsive to the previous therapy. In some embodiments, the antidepressant is an atypical antidepressant, a selective serotonin reuptake inhibitor, a selective serotonin and norepinephrine reuptake inhibitor, a monoamine oxidase inhibitor, a selective norepinephrine reuptake inhibitor, or a tricyclic antidepressant, and was not responsive to the previous therapy.

In some embodiments, the subject has previously been administered one or more atypical antidepressants, such as mirtazapine, mianserin, bupropion, trazodone, nefazodone, tianeptine, opipramol, agomelatine, vilazodone, and vortioxetine, and was not responsive to the previous therapy.

In some embodiments, the subject has previously been administered one or more selective serotonin reuptake inhibitors, such as citalopram, escitalopram, fluoxetine, fluvoxamine, paroxetine, and sertraline, and was not responsive to the previous therapy.

In some embodiments, the subject has previously been administered one or more selective serotonin and norepinephrine reuptake inhibitors, such as atomoxetine, desvenlafaxine, duloxetine, levomilnacipran, milnacipran, sibutramine, tramadol, and venlafaxine, and was not responsive to the previous therapy.

In some embodiments, the subject has previously been administered one or more monoamine oxidase inhibitors, such as moclobemide, rasagiline, selegiline, or safinamide, and was not responsive to the previous therapy.

In some embodiments, the subject has previously been administered one or more selective norepinephrine reuptake inhibitors, such as reboxetine, and was not responsive to the previous therapy.

In some embodiments, the subject has previously been administered one or more tricyclic antidepressants, such as amineptine, amitriptyline, amoxapine, butriptyline, clomipramine, desipramine, dibenzepin, dosulepin, doxepin, imipramine, iprindole, lofepramine, maprotiline, norclomipramine, northiaden, nortriptyline, pipramol, protriptyline, tianeptine, and trimipramine, and was not responsive to the previous therapy.

In some embodiments, the subject has previously been administered one or more benzodiazepines, such as alprazolam, bromazepam, chlordiazepoxide, clonazepam, clorazepate, diazepam, flurazepam, lorazepam, oxazepam, temazepam, or triazolam, and was not responsive to the previous therapy.

In some embodiments, the subject has previously been administered one or more mood stabilizers, such as lithium, valproic acid, lamotrigine, or carbamazepine, and was not responsive to the previous therapy.

In some embodiments, the one or more additional therapies is electroconvulsive therapy or transcranial magnetic stimulation, and was not responsive to the previous therapy.

In some embodiments, the subject has previously been administered sertraline, and was not responsive to the previous therapy. In some embodiments, the subject has previously been administered venlafaxine, and was not responsive to the previous therapy.

In some embodiments, the subject has previously been administered one or more cholinesterase inhibitors such as donepezil, galantamine, or rivastigmine, and was not responsive to the previous therapy.

In some embodiments, the subject has previously been administered memantine, and was not responsive to the previous therapy.

In some embodiments, the subject has previously been administered one or more NSAIDs such as clonixin, licofelone, aspirin, diflunisal, ibuprofen, dexibuprofen, naproxen, fenoprofen, ketoprofen, dexketoprofen, flurbiprofen, oxaprozin, indomethacin, tolmetin, sulindac, etodolac, ketorolac, diclofenac, aceclofenac, bromfenac), or celecoxib, and was not responsive to the previous therapy.

In some embodiments, the subject has previously been administered one or more analgesics such as nefopam, flupiritine, ziconotide, acetaminophen, morphine, oxycodone, methadone, codeine, fentanyl, hydrocodone, or tramadol, and was not responsive to the previous therapy. In some embodiments, the subject has previously been administered one or more anxiolytics, such as alnespirone, adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, or zolazepam, and was not responsive to the previous therapy.

In some embodiments, the subject has previously been administered gabapentin or pregabalin, and was not responsive to the previous therapy.

In some embodiments, the one or more additional therapies previously administered to the subject is 1-3 additional therapies. In some embodiments, the one or more additional therapies previously administered to the subject is one additional therapy. In some embodiments, the one or more additional therapies previously administered to the subject is two additional therapies. In some embodiments, the one or more additional therapies previously administered to the subject is three additional therapies.

Subjects that were “non-responsive” to a previous therapy includes subjects where the previous therapy lacked sufficient clinical efficacy, subjects that experienced an unacceptable number and/or severity of side effects due to the previous therapy (sufficient to require discontinuation of treatment), and subjects that experienced both of the foregoing. Side effects include, but are not limited to weight gain, flattened affect, tardive dyskinesia, drowsiness, nausea, vomiting, constipation, dry mouth, restlessness, dizziness, loss of sexual desire, erectile dysfunction, insomnia, and blurred vision.

### EMBODIMENTS

### Exemplary Embodiments of Compounds of Formula (I)

### Embodiment 1: A Compound of Formula (I)

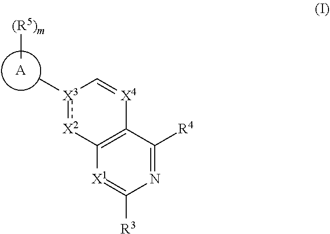

or a pharmaceutically acceptable salt thereof, wherein:
each dashed line represents a single bond or a double bond;
X¹ is CR¹ or N;
X² is CR², C(═O), or N;
X³ is C or N; provided that when X² is C(═O), X³ is N;
X⁴ is CH or N;
Ring A is phenyl or 5-6 membered heteroaryl;
R¹ is hydrogen, halogen, cyano, 3-10 membered heterocyclyl, C1-C6 alkyl optionally substituted with 3-6 membered heterocyclyl optionally substituted with —C(═O)C1-C6 alkyl or —C(═O)OR^{A}, C1-C6 alkoxy, —C(═O)-3-6 membered heterocyclyl optionally substituted with C1-C6 alkyl, or —OR^{B};
R^{B} is a 3-6 membered heterocyclyl, a 5-6 membered heteroaryl, a C6-C10 aryl, or a C3-C6 cycloalkyl each optionally substituted with 1-3 independently selected halogen, C1-C6 alkyl, C1-C6 haloalkyl, C1-C6 alkoxy, cyano, hydroxy, —C(═O)OH, —C(═O)C1-C6 alkyl, —S(O₂)—C1-C6 alkyl, or —NR^{C}R^{D};
R² is hydrogen, halogen, C1-C6 alkyl, C1-C6 alkoxy, —C(═O)-3-6 membered heterocyclyl, —NH—C3-C6 cycloalkyl-C(═O)OR^{A}, or —O—C3-C6 cycloalkyl-C(═O)OR^{A};
R³ is hydrogen, halogen, C1-C6 alkyl, cyano, C3-C6 cycloalkyl, —X—R^{G}, or

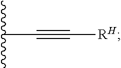

R⁴ is hydrogen or C1-C6 alkyl;
R⁵ is hydrogen, C1-C6 alkyl optionally substituted with 3-6 membered heterocyclyl; —X—R^{E}; —C3-C6 cycloalkyl-C(═O)OR^{A}; or

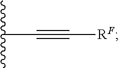

or
R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a (i) C6-C10 aryl optionally substituted with a 3-10 membered heterocyclyl optionally substituted with 1-2 independently selected C1-C6 alkyl or —C(═O)OR′; (ii) a 3-6 membered heterocyclyl; or a (iii) a 5-6 membered heteroaryl optionally substituted with 1-2 substituents independently selected from C1-C6 alkyl, C1-C6 haloalkyl, C1-C6 alkoxy, 3-10 membered heterocyclyl optionally substituted with 1-2 independently selected C1-C6 alkyl or C(═O)OR′, and 5-6 membered heteroaryl optionally substituted with C1-C6 alkyl;
R^{E} is a 3-10 membered heterocyclyl, a 5-6 membered heteroaryl, or a C3-C6 cycloalkyl each optionally substituted with 1-3 independently selected C1-C6 alkyl optionally substituted with C1-C6 alkoxy, NR^{I}R^{J}, or —C(═O)OH;
R^{F} is a 3-6 membered heterocyclyl, a 5-6 membered heteroaryl, or a C3-C6 cycloalkyl each optionally substituted with C1-C6 alkyl; or a C2-C6 alkynyl optionally substituted with hydroxy;
R^{G} is a 3-6 membered heterocyclyl or a C3-C6 cycloalkyl each optionally substituted with 1-3 independently selected C1-C6 alkyl, —C(═O)C1-C6 alkyl, —C(═O)OH, or NR^{C}R^{D};
R^{H} is a C1-C6 alkyl optionally substituted with hydroxy or a 3-6 membered heterocyclyl optionally substituted with C1-C6 alkyl;
X is —NH—, —NH(C═O)—, —NHC(═O)O—, —O—, —(C═O)— or CH₂;
each R^{A}, R^{C}, R^{D}, R^{I}, and R^{J} is independently selected from hydrogen and C1-C6 alkyl; and
m is 0, 1, or 2.

Embodiment 2: The compound of Embodiment 1, wherein one of X¹, X², X³, X⁴ is N, and each one of the remaining of X¹, X², X³, X⁴ is independently selected from C, C(═O), CH, CR¹ or CR².

Embodiment 3: The compound of Embodiment 1 or 2, wherein X¹ is N; X² is CR²; X³ is C; and X⁴ is CH.

Embodiment 4: The compound of Embodiment 1 or 2, wherein X² is N; X¹ is CRi; X³ is C; and X⁴ is CH.

Embodiment 4: The compound of Embodiment 1 or 2, wherein X³ is N; X¹ is CR¹; X² is C(═O); and X⁴ is CH.

Embodiment 6: The compound of Embodiment 1 or 2, wherein X⁴ is N; X¹ is CR¹; X² is CR²; and X³ is C.

Embodiment 7: The compound of Embodiment 1, wherein each one of X¹, X², X³, X⁴ is independently selected from C, C(═O), CH, CR¹ or CR².

Embodiment 8: The compound of Embodiment 1, wherein ring A is a 5-membered heteroaryl.

Embodiment 9: The compound of any one of Embodiments 1-8, wherein Ring A is

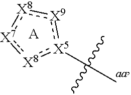

wherein aa represents the point of connection to X³, and each dash bond is independently a single bond or a double, and each one of X⁵, X⁶, X⁷, X⁸ and X⁹ is independently selected from C, (C═O), C═NH, CH, N, O, or S.

Embodiment 10: The compound of any one of Embodiments 1-9, wherein Ring A is selected from the group consisting of thiazolyene, oxazolyene, imidazolyene, pyrazolyene, 1,2,4-triazolyene, 1,2,4-oxadiazolylene and 2-imine-thiazolylene.

Embodiment 11: The compound of any one of Embodiments 1-9, wherein Ring A is selected from the group consisting of

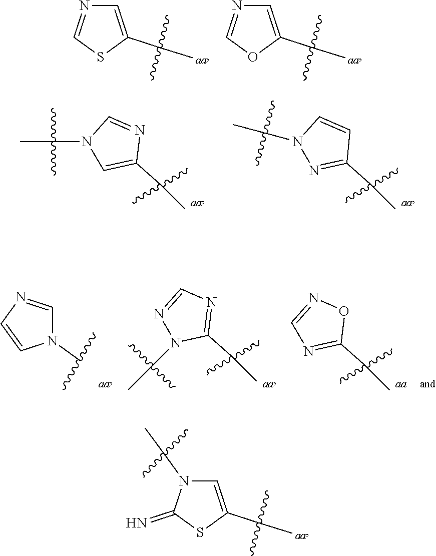

each of which is optionally substituted with one or two R⁵, and aa represents the point of attachment to X³ and the other wave line represents the point of connection to R⁵.

Embodiment 12: The compound of any one of Embodiments 1-7, wherein Ring A is a 6-membered heteroaryl.

Embodiment 13: The compound of any one of Embodiments 1-7 or 12, wherein Ring A is

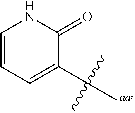

wherein aa represents the point of attachment to X³.

Embodiment 14: The compound of any one of Embodiments 1-13, wherein R¹ is hydrogen.

Embodiment 15: The compound of any one of Embodiments 1-13, wherein R¹ is cyano.

Embodiment 16: The compound of any one of Embodiments 1-13, wherein R¹ is halo (e.g., Cl or F).

Embodiment 17: The compound of any one of Embodiments 1-13, wherein R¹ is 3-10 membered heterocyclyl.

Embodiment 18: The compound of Embodiment 17, wherein R¹ is

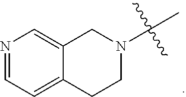 [SMILES: *N1CCc2ccncc2C1]

Embodiment 19: The compound of any one of Embodiments 1-13, wherein R¹ is C1-C6 alkyl optionally substituted with 3-6 membered heterocyclyl optionally substituted with —C(═O)C1-C6 alkyl or —C(═O)OR^{A}.

Embodiment 20: The compound of any of Embodiments 1-13 or 19, wherein R¹ is C1-C6 alkyl (e.g., methyl).

Embodiment 21: The compound of any one of Embodiments 1-13 or 19, wherein R¹ is C1-C6 alkyl substituted with 3-6 membered heterocyclyl.

Embodiment 22: The compound of any one of Embodiments 1-13 or 21, wherein R¹ is C1-C6 alkyl substituted with 3-6 membered heterocyclyl substituted with C(═O)OR^{A}.

Embodiment 23: The compound of any one of Embodiments 1-13 or 21, wherein R¹ is C1-C6 alkyl substituted with 3-6 membered heterocyclyl substituted with —C(═O)C1-C6 alkyl.

Embodiment 24: The compound of any one of Embodiments 21-23, wherein the heterocyclyl is piperidinylene, piperidinyl, piperizinylene, or piperizinyl.

Embodiment 25: The compound of Embodiment 22, where R¹ is

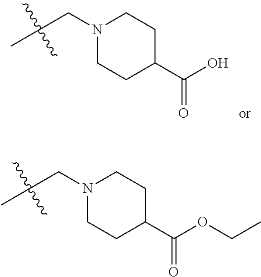 [SMILES: *CN1CCC(C(=O)O)CC1 ; "or" ; *CN1CCC(C(=O)OCC)CC1]

wherein the wave line represent the point of attachment to X¹.

Embodiment 26: The compound of Embodiment 23, wherein R¹ is

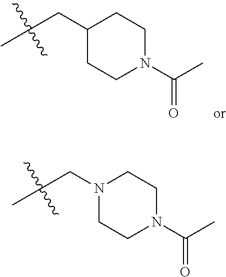 [SMILES: *CC1CCN(C(C)=O)CC1 ; "or" ; *CN1CCN(C(C)=O)CC1]

wherein the wave line represent the point of attachment to X¹.

Embodiment 27: The compound of any one of Embodiments 1-13 wherein R¹ is C1-C6 alkoxy (e.g., methoxy).

Embodiment 28: The compound of any one of Embodiments 1-13, wherein R¹ is —C(═O)-3-6 membered heterocyclyl optionally substituted with C1-C6 alkyl.

Embodiment 29: The compound of Embodiment 28, wherein R¹ is

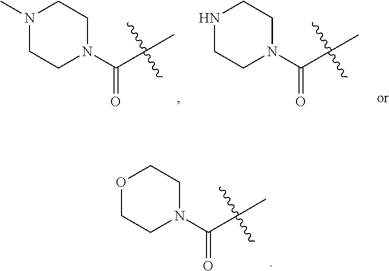

Embodiment 30: The compound of Embodiments 1-13, wherein R¹ is —OR^{B}.

Embodiment 31: The compound Embodiment 30, wherein R¹ is selected from the group consisting of

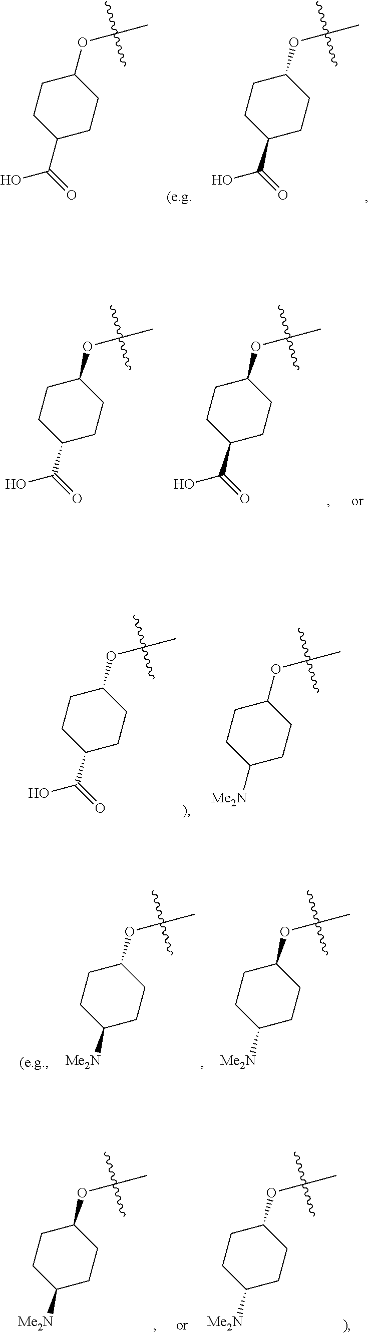

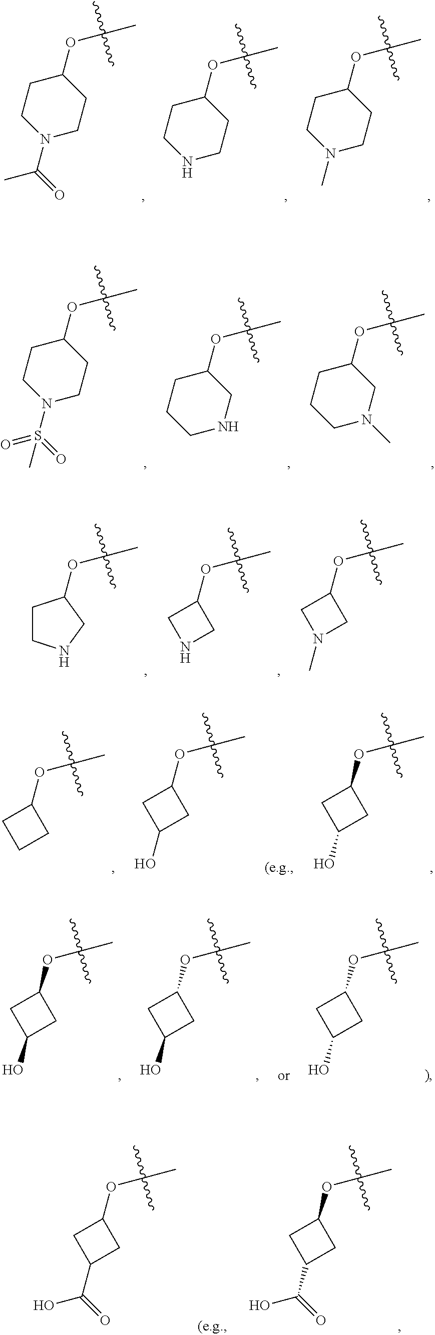

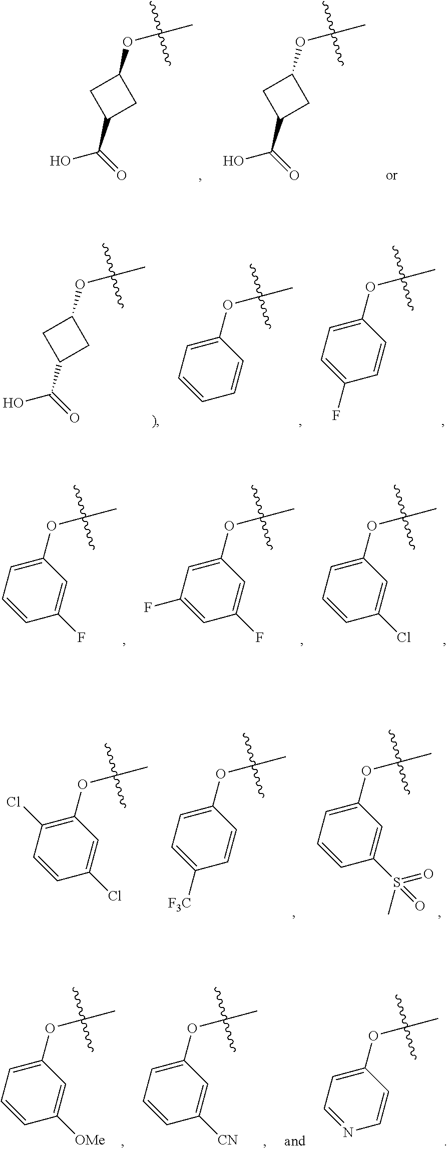

Embodiment 32: The compound of any one of Embodiments 1-31, wherein R² is hydrogen.

Embodiment 33: The compound of any one of Embodiments 1-31, wherein R² is halogen (e.g., F, C1).

Embodiment 34: The compound of any one of Embodiments 1-31, wherein R² is C1-C6 alkyl (e.g., methyl).

Embodiment 35: The compound of any one of Embodiments 1-31, wherein R² is C1-C6 alkoxy (e.g., methoxy, ethoxy).

Embodiment 36: The compound of any one of Embodiments 1-31, wherein R² is —C(═O)-3-6 membered heterocyclyl.

Embodiment 37: The compound of any one of Embodiments 1-31, wherein R² is —NH—C3-C6 cycloalkyl-C(═O)OR^{A}.

Embodiment 38: The compound of any one of Embodiments 1-31, wherein R² is or —O—C3-C6 cycloalkyl-C(═O)OR^{A}.

Embodiment 39: The compound of any one of Embodiments 1-38, wherein R³ is hydrogen.

Embodiment 40: The compound of any one of Embodiments 1-38, wherein R³ is halogen (e.g., F or C1).

Embodiment 41: The compound of any one of Embodiments 1-38, wherein R³ is cyano.

Embodiment 42: The compound of any one of Embodiments 1-38, wherein R³ is C1-C6 alkyl (e.g., methyl, ethyl).

Embodiment 43: The compound of any one of Embodiments 1-38, wherein R³ is C3-C6 cycloalkyl (e.g., cyclopropyl).

Embodiment 44: The compound of any one of Embodiments 1-38, wherein R³ is —X—R^{G} wherein X is —NH—, —NHC(═O)O—, —O—, or CH₂.

Embodiment 45: The compound of Embodiment 44, wherein R³ is selected from the list of structures consisting of

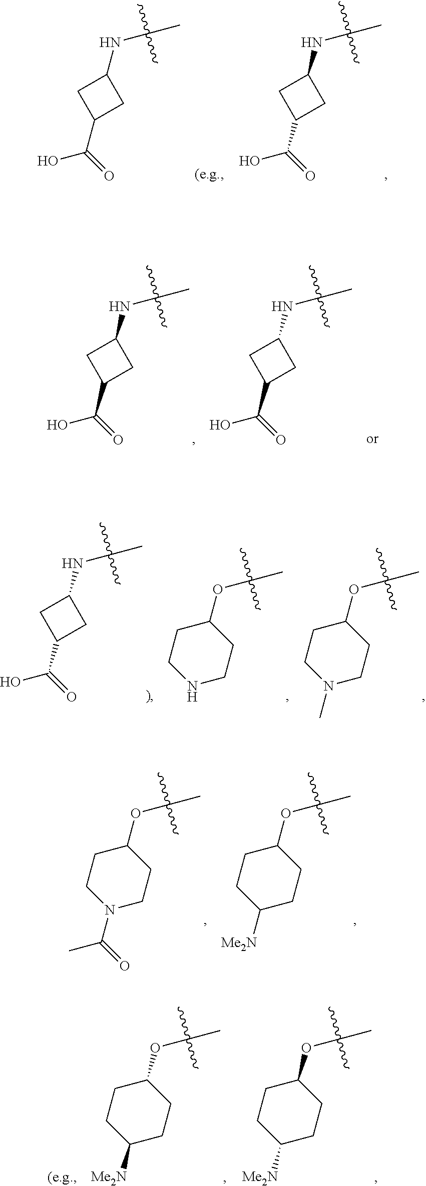

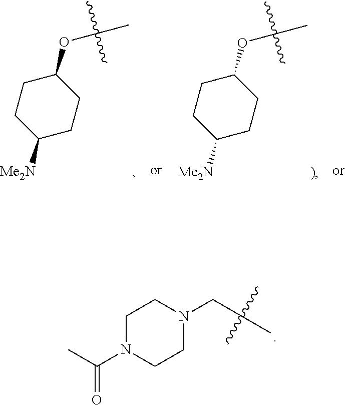 [SMILES: *O[C@H]1CC[C@@H](N(C)C)CC1 ; "or" ; *O[C@H]1CC[C@@H](N(C)C)CC1 ; "or" ; *CN1CCN(C(C)=O)CC1 ; "."]

Embodiment 46: The compound of any one of Embodiments 1-38, wherein R³ is

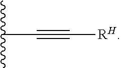

Embodiment 47: The compound of Embodiment 46, wherein R³ is selected from the group consisting of

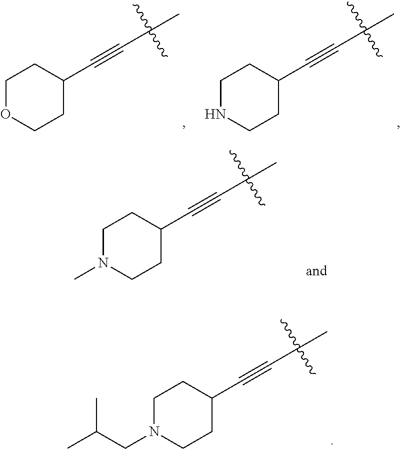

Embodiment 48: The compound of any one of Embodiments 1-47, wherein R⁴ is hydrogen.

Embodiment 49: The compound of any one of Embodiments 1-47, wherein R⁴ is C1-C6 alkyl (e.g., methyl, ethyl).

Embodiment 50: The compound of any one of Embodiments 1-49, wherein m is 0.

Embodiment 51: The compound of any one of Embodiments 1-49, wherein m is 1 or 2.

Embodiment 52: The compound of any one of Embodiments 1-49 or 51, wherein m is 1.

Embodiment 53: The compound of Embodiment 52, wherein R⁵ is hydrogen.

Embodiment 64: The compound of Embodiment 52, wherein R⁵ is C1-C6 alkyl optionally substituted with 3-6 membered heterocyclyl.

Embodiment 55: The compound of Embodiment 54, wherein R⁵ is C1-C6 alky (e.g., methyl, ethyl).

Embodiment 56: The compound of Embodiment 54, wherein R⁵ is C1-C6 alkyl substituted with 3-6 membered heterocyclyl.

Embodiment 57: The compound of 54, wherein the heterocyclyl group is morpholinyl (e.g.,

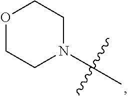 [SMILES: *N1CCOCC1]

wherein the wave line represents the point of connection to the C1-C6 alkyl group).

Embodiment 58: The compound of any one of Embodiments 54, 56 or 57, wherein R⁵ is

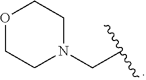 [SMILES: *CN1CCOCC1]

Embodiment 59: The compound of any one of Embodiments 1-49, 51 or 52, wherein R⁵ is —X—R^{E}.

Embodiment 60: The compound of Embodiment 59, wherein R⁵ is —(NH)(C═O)R^{E}, —(NH)(C═O)O—R^{E}, —NH—R^{E}, or —(C═O)R^{E}—.

Embodiment 61: The compound of Embodiment 59 or 60, wherein R⁵ is selected from the group consisting of

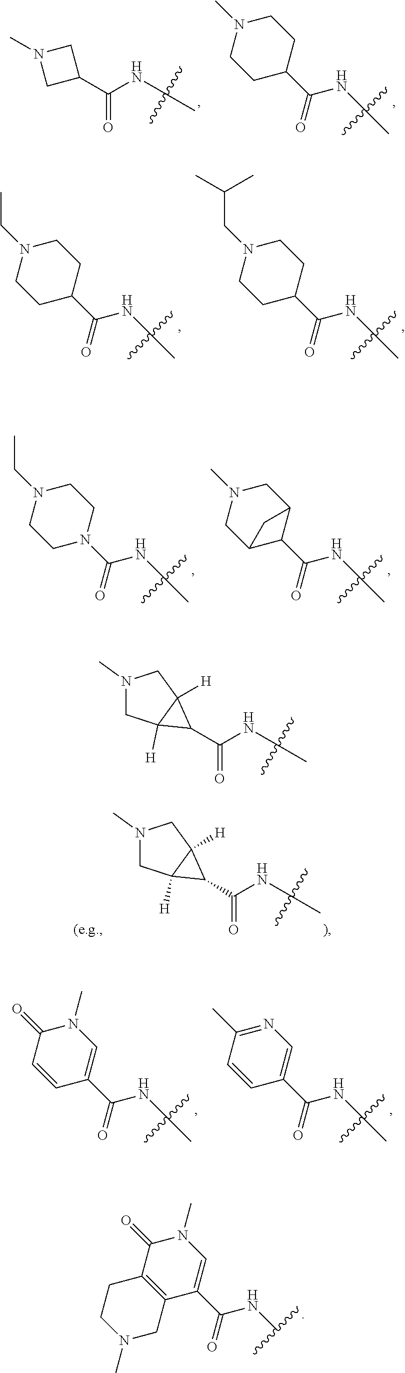

Embodiment 62: The compound of Embodiment 59 or 60, wherein R⁵ is

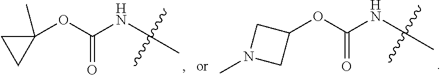

Embodiment 63: The compound of Embodiment, 59 or 60, wherein R⁵ is

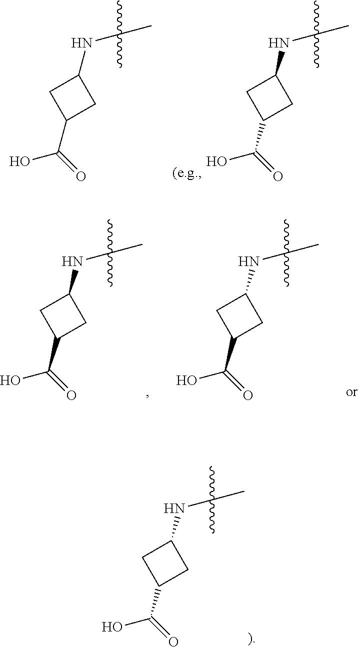

Embodiment 64: The compound of Embodiment 59 or 60, wherein R⁵ is

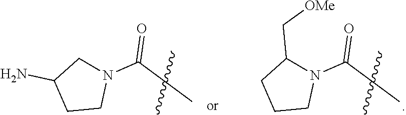 [SMILES: *C(=O)N1CCC(N)C1 ; *C(=O)N1CCCC1COC]

Embodiment 65: The compound of Embodiment 1-49, 51 or 52, wherein R⁵ is —C3-C6 cycloalkyl-C(═O)OR^{A}.

Embodiment 66: The compound of Embodiment 65, wherein R⁵ is

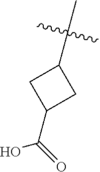 [SMILES: *C1CC(C(=O)O)C1]

(e.g.,

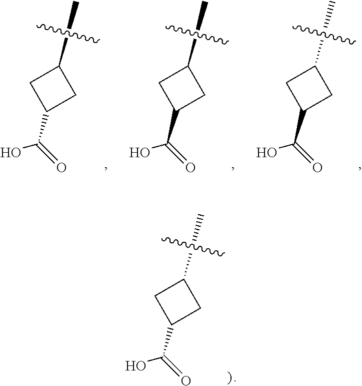

Embodiment 67: The compound of any one of Embodiments 1-49, 51, or 52, wherein R⁵

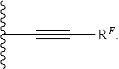

Embodiment 68: The compound of Embodiment 67, wherein R^{F} is a 3-6 membered heterocyclyl optionally substituted with C2-C6 alkynyl.

Embodiment 69: The compound of Embodiment 67 or 68, wherein R⁵ is selected from the group consisting of

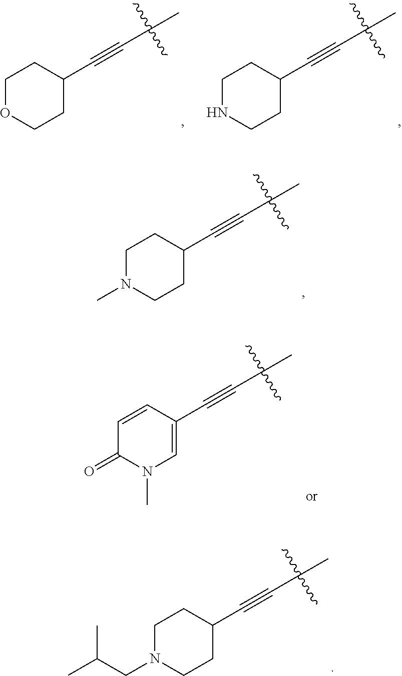

Embodiment 70: The compound of Embodiment 67, wherein R^{F} is C2-C6 alkynyl optionally substituted with hydroxy.

Embodiment 71: The compound of Embodiment 67 or 70, wherein R⁵ is

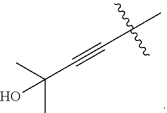 [SMILES: *C#CC(C)(C)O]

Embodiment 72: The compound of any one of Embodiments 1-49, 51 or 52, wherein R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a (i) C6-C10 aryl optionally substituted with a 3-10 membered heterocyclyl optionally substituted with 1-2 independently selected C1-C6 alkyl or —C(═O)OR′; (ii) a 3-6 membered heterocyclyl; or a (iii) a 5-6 membered heteroaryl optionally substituted with 1-2 substituents independently selected from C1-C6 alkyl, C1-C6 haloalkyl, C1-C6 alkoxy, 3-10 membered heterocyclyl optionally substituted with 1-2 independently selected C1-C6 alkyl or C(═O)OR′, and 5-6 membered heteroaryl optionally substituted with C1-C6 alkyl;

Embodiment 73: The compound of Embodiment 72, wherein R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a C6-C10 aryl (e.g., phenyl) optionally substituted with a 3-10 membered heterocyclyl optionally substituted with 1-2 independently selected C1-C6 alkyl or —C(═O)OR′.

Embodiment 74: The compound Embodiment 72 or 73, wherein R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a phenyl ring (e.g.,

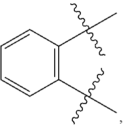 [SMILES: *c1ccccc1*]

wherein the wavy lines represent the point of attachment to Ring A), which is further optionally substituted with optionally substituted with a 3-10 membered heterocyclyl optionally substituted with 1-2 independently selected C1-C6 alkyl or —C(═O)OR′.

Embodiment 75: The compound of any one of Embodiments 72-74, wherein R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a structure selected from a group consisting of

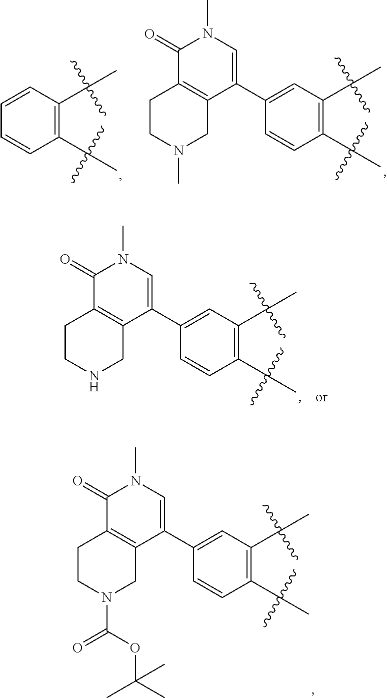 [SMILES: *c1ccccc1* ; *c1ccc(-c2cn(C)c(=O)c3c2CN(C)CC3)cc1* ; *c1ccc(-c2cn(C)c(=O)c3c2CNCC3)cc1* ; *c1ccc(-c2cn(C)c(=O)c3c2CN(C(=O)OC(C)(C)C)CC3)cc1*]

wherein the wavy lines represent the points of attachment to Ring A.

Embodiment 76: The compound of Embodiment 72, wherein R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a 3-6 membered heterocyclyl.

Embodiment 77: The compound of Embodiment 75, wherein R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a pyrrolidine ring (e.g.,

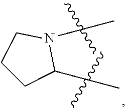 [SMILES: *C1CCCN1*]

wherein the wavy lines represent the points of attachment to Ring A).

Embodiment 78: The compound of Embodiment 72, wherein R⁵ and the carbon and/or nitrogen atom to which it is attached, forms a bond with an adjacent carbon or nitrogen atom, replacing the hydrogen atom on the adjacent carbon or nitrogen atom, and R⁵ and the two adjacent carbon and/or nitrogen atoms in Ring A from a 5-6 membered heteroaryl optionally substituted with 1-2 substituents independently selected from C1-C6 alkyl, C1-C6 haloalkyl, C1-C6 alkoxy, 3-10 membered heterocyclyl optionally substituted with 1-2 independently selected C1-C6 alkyl or C(═O)OR′, and 5-6 membered heteroaryl optionally substituted with C1-C6 alkyl.

Embodiment 79: The compound of Embodiment 72 or 78, wherein R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A from a ring structure selected from the list of structures consisting of

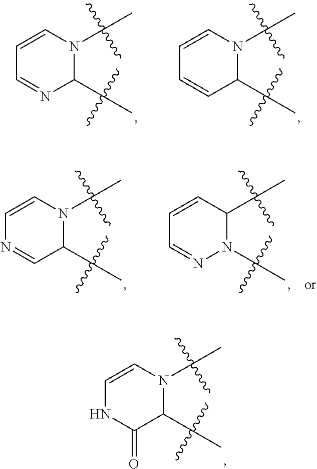 [SMILES: *C1N=CC=CN1* ; *C1C=CC=CN1* ; *C1C=NC=CN1* ; *C1C=CC=NN1* ; *C1C(=O)NC=CN1*]

each of which is optionally substituted with 1-2 substituents independently selected from C1-C6 alkyl, C1-C6 haloalkyl, C1-C6 alkoxy, 3-10 membered heterocyclyl optionally substituted with 1-2 independently selected C1-C6 alkyl or C(═O)OR′, and 5-6 membered heteroaryl optionally substituted with C1-C6 alkyl, and the wavy lines represent the points of attachment to Ring A.

Embodiment 80: The compound of any one of Embodiments 72, 78 or 79, wherein R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A from a ring structure selected from the list of structures consisting of

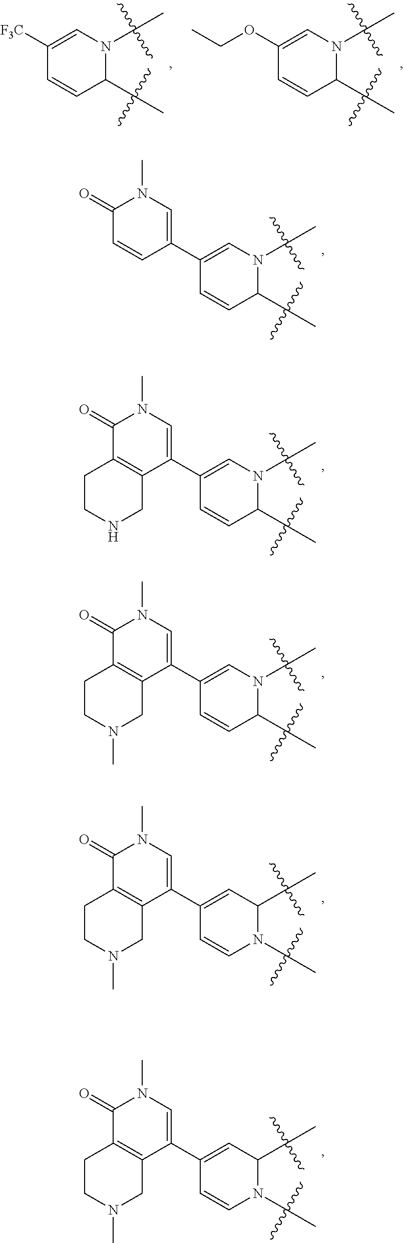

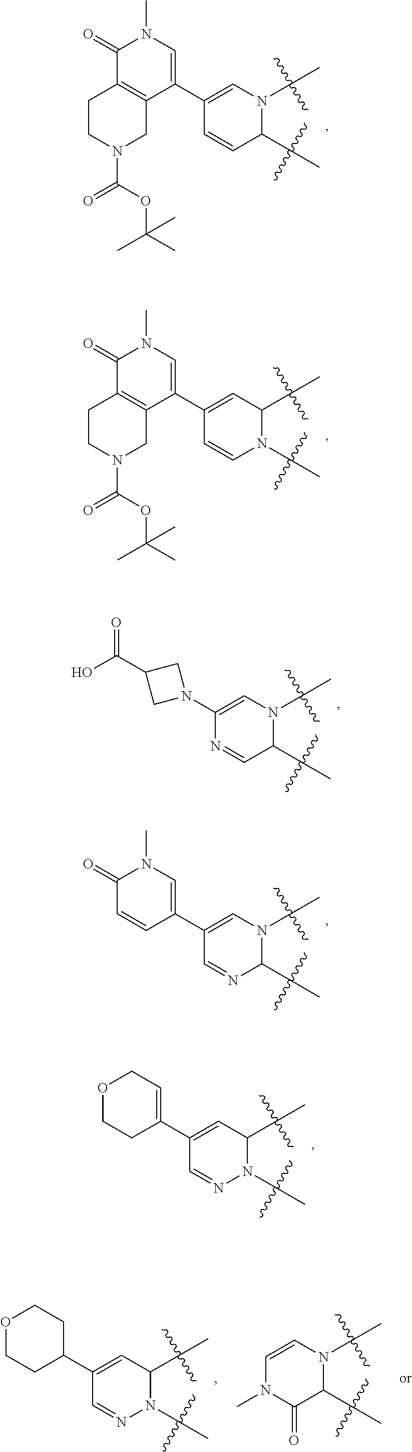

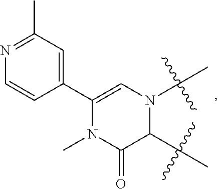 [SMILES: *C1C(=O)N(C)C(c2ccnc(C)c2)=CN1*]

wherein the wavy lines represent the points of attachment to Ring A.

Embodiment 81: The compound of Embodiment 1, wherein the compound is a compound of formula (I-1), or a pharmaceutically acceptable salt thereof:

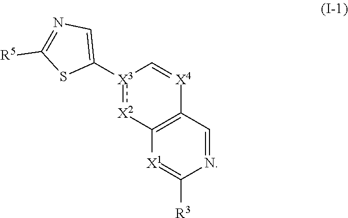

Embodiment 82: The compound of Embodiment 1, wherein the compound is a compound of formula (I-2), or a pharmaceutically acceptable salt thereof:

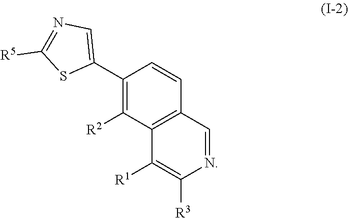

Embodiment 83: The compound of Embodiment 1, wherein the compound is a compound of formula (I-3), or a pharmaceutically acceptable salt thereof:

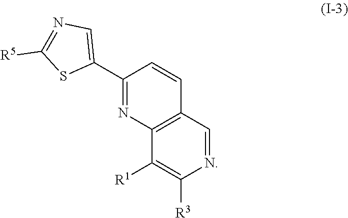

Embodiment 84: The compound of Embodiment 1, wherein the compound is a compound of formula (I-4), or a pharmaceutically acceptable salt thereof:

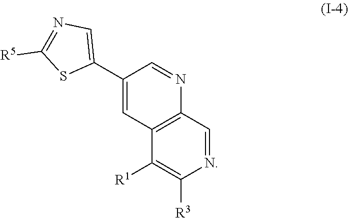

Embodiment 85: The compound of Embodiment 1, wherein the compound is a compound of formula (I-5), or a pharmaceutically acceptable salt thereof:

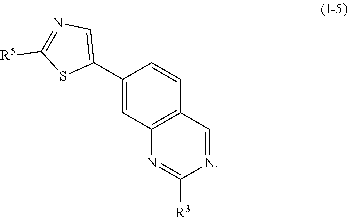

Embodiment 86: The compound of Embodiment 1, wherein the compound is a compound of formula (I-6), or a pharmaceutically acceptable salt thereof:

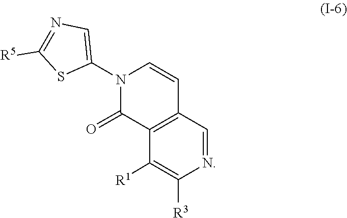

Embodiment 87: The compound of Embodiment 1, wherein the compound is a compound of formula (I-7), or a pharmaceutically acceptable salt thereof:

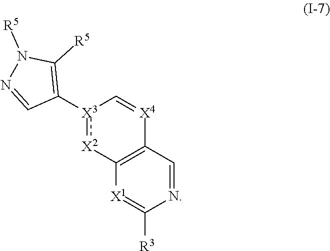

Embodiment 88: The compound of Embodiment 1, wherein the compound is a compound of formula (I-8), or a pharmaceutically acceptable salt thereof:

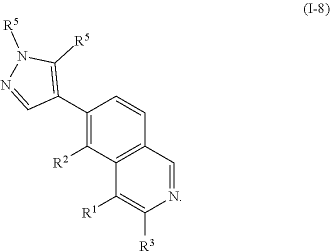

Embodiment 89: The compound of Embodiment 1, wherein the compound is a compound of formula (I-9), or a pharmaceutically acceptable salt thereof:

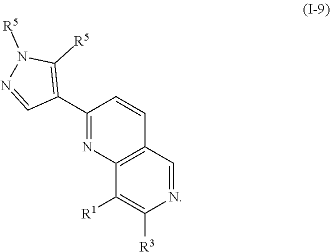

Embodiment 90: The compound of Embodiment 1, wherein the compound is a compound of formula (I-10), or a pharmaceutically acceptable salt thereof:

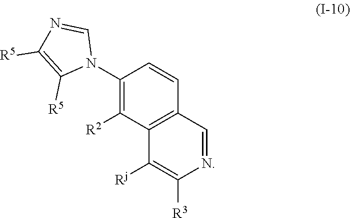

Embodiment 91: The compound of Embodiment 1, wherein the compound is a compound of formula (I-11), or a pharmaceutically acceptable salt thereof:

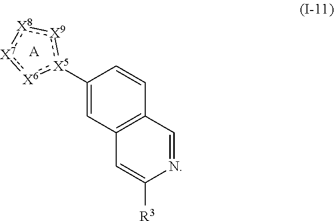

Embodiment 92: The compound of Embodiment 1, wherein the compound is a compound of formula (I-12), or a pharmaceutically acceptable salt thereof:

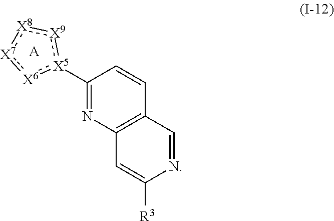

Embodiment 93: The compound of Embodiment 1, wherein the compound is a compound of formula (I-13), or a pharmaceutically acceptable salt thereof:

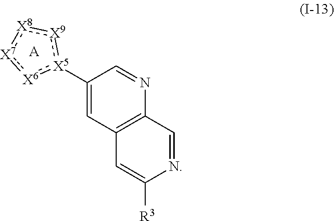

Embodiment 94: The compound of Embodiment 1, wherein the compound is a compound of formula (I-14), or a pharmaceutically acceptable salt thereof:

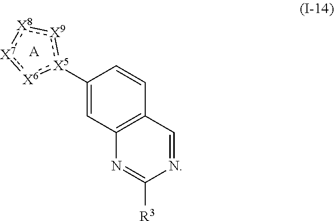

Embodiment 95: The compound of Embodiment 1, wherein the compound is a compound of formula (I-15), or a pharmaceutically acceptable salt thereof:

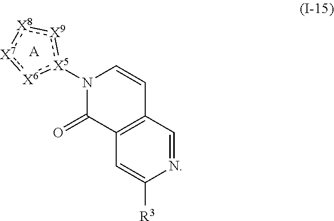

### Exemplary Embodiments of Compounds of Formula (II)

### Embodiment 1: A Compound of Formula (II)

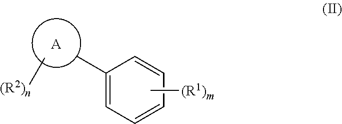

or a pharmaceutically acceptable salt thereof, wherein:
Ring A is a 5-14 membered heteroaryl or a 5-14 membered heterocyclyl;
each R¹ is independently halogen, hydroxyl, cyano, C1-C6 alkyl, C1-C6 alkoxy, —C(═O)OR^{A}, —NR^{B}R^{C}, and —C(═O)NR^{B}R^{C};
each R² is independently —C(═O)OR^{D}, C1-C6 alkyl, C2-C6 alkynyl optionally substituted with 4-8 membered heterocyclyl optionally substituted with C1-C6 alkyl, —C(═O)-phenyl, —(C1-C6 alkyl)-phenyl, —(C1-C6 alkyl)-4-10 membered heterocyclyl optionally substituted with C1-C6 alkyl, 4-10 membered heterocyclyl optionally substituted with C1-C6 alkyl or —CO₂C1-C6 alkyl, phenyl optionally substituted with cyano or fluoro, —NHC(═O)R^{E}, 5-6 membered heteroaryl optionally substituted with C1-C6 alkoxy,
m is 1, 2, or 3;
n is 0, 1, 2, or 3;
each R^{A}, R^{B}, R^{C}, and R^{D} is independently hydrogen or C1-C6 alkyl; and
each R^{E} is independently C3-C6 cycloalkyl, 4-8 membered heterocyclyl optionally substituted with C1-C6 alkyl, or 5-6 membered heteroaryl optionally substituted with C1-C6 alkyl.

Embodiment 2: The compound of Embodiment 1, wherein Ring A is a monocyclic heteroaryl or monocyclic heterocyclyl.

Embodiment 3: The compound of Embodiment 1 or 2, wherein Ring A is a 5-6 membered heteroaryl.

Embodiment 4: The compound of any one of Embodiments 1-3, wherein Ring A is a 5-membered heteroaryl.

Embodiment 5: The compound of any one of Embodiments 1-4, wherein Ring A is thiazole or pyrazole.

Embodiment 6: The compound of any one of Embodiments 1-4, wherein Ring A is

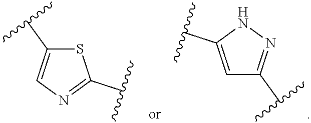 [SMILES: *c1cnc(*)s1 ; *c1cc(*)[nH]n1]

Embodiment 7: The compound of Embodiment 1 or 2, wherein Ring A is a 6-membered heteroaryl.

Embodiment 8: The compound of any one of Embodiments 1-2 or 7, wherein Ring A is pyridine or pyrimidin-4(3H)-one.

Embodiment 9: The compound of any one of Embodiments 1-2 or 7, wherein Ring A is

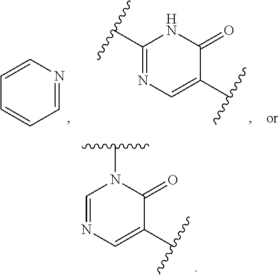 [SMILES: c1ccncc1 ; *c1ncc(*)c(=O)[nH]1 ; *c1cncn(*)c1=O]

Embodiment 10: The compound of Embodiment 1, wherein Ring A is a bicyclic heteroaryl or a bicyclic heterocyclyl.

Embodiment 11: The compound of Embodiment 1 or 10, wherein Ring A is an 8-12 membered bicyclic heteroaryl or 8-12 membered bicyclic heterocyclyl.

Embodiment 12: The compound of any one of Embodiments 1 or 10-11, wherein Ring A is a 9-10 membered bicyclic heteroaryl or 9-10 membered bicyclic heterocyclyl.

Embodiment 13: The compound of any one of Embodiments 1 or 10-12, wherein Ring A is a 9-membered bicyclic heteroaryl.

Embodiment 14: The compound of any one of Embodiments 1 or 10-13, wherein Ring A is pyrazolo[1,5-a]pyridine, 1H-pyrrolo[2,3-b]pyridine, pyrrolo[1,2-a]pyrazin-1(2H)-one, pyrazolo[1,5-a]pyrazine, imidazo[1,2-b]pyridazine, pyrazolo[1,5-a]pyrimidine, or 1,3-dihydro-2H-pyrrolo[2,3-b]pyridin-2-one.

Embodiment 15: The compound of any one of Embodiments 1 or 10-13, wherein Ring A is

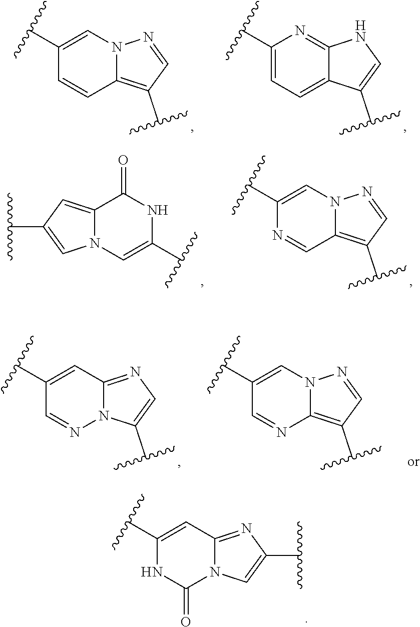

Embodiment 16: The compound of any one of Embodiments 1 or 10-12, wherein Ring A is a 9-membered bicyclic heterocyclyl.

Embodiment 17: The compound of any one of Embodiments 1, 10-12 or 16, wherein Ring A is 5,6,7,8-tetrahydroimidazo[1,2-a]pyrazine, 1,3-dihydro-2H-pyrrolo[2,3-b]pyridin-2-one, or 1,3-dihydro-2H-imidazo[4,5-b]pyridin-2-one.

Embodiment 18: The compound of any one of Embodiments 1, 10-12 or 16, wherein Ring A is

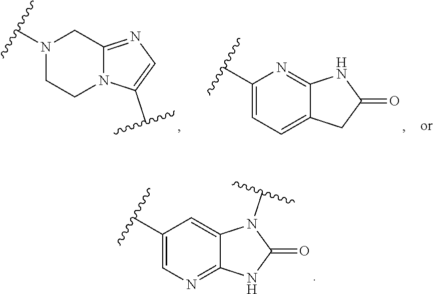 [SMILES: *c1cnc2n1CCN(*)C2 ; *c1ccc2c(n1)NC(=O)C2 ; *c1cnc2[nH]c(=O)n(*)c2c1]

Embodiment 19: The compound of Embodiment 1, wherein Ring A is a tricyclic heteroaryl or a tricyclic heterocyclyl.

Embodiment 20: The compound of Embodiment 1 or 19, wherein Ring A is a 10-14 membered tricyclic heteroaryl or a 10-14 membered tricyclic heterocyclyl.

Embodiment 21: The compound of any one of Embodiments 1 or 19-20, wherein Ring A is a 11-13 membered tricyclic heteroaryl or a 11-13 membered tricyclic heterocyclyl.

Embodiment 22: The compound of any one of Embodiments 1 or 19-21, wherein Ring A is a 12-membered tricyclic heteroaryl.

Embodiment 23: The compound of any one of Embodiments 1 or 19-22, wherein Ring A is 8H-pyrazolo[1,5-a]pyrrolo[3,2-e]pyrimidine.

Embodiment 24: The compound of any one of Embodiments 1 or 19-22, wherein Ring A is

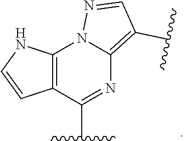 [SMILES: *c1nc2c(*)cnn2c2[nH]ccc12]

Embodiment 25: The compound of any one of Embodiments 1 or 19-21, wherein Ring A is a 12-membered tricyclic heterocyclyl.

Embodiment 26: The compound of any one of Embodiments 1, 19-21 or 25, wherein Ring A is 7,8,9,10-tetrahydro-pyrazolo[5,1-f][1,6]naphthyridine, or 7,8-dihydro-6H-pyrazolo[1,5-a]pyrrolo[3,2-e]pyrimidine.

Embodiment 27: The compound of Embodiment 1, 19-21 or 25, wherein Ring A is

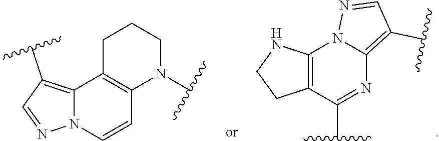 [SMILES: *c1cnn2ccc3c(c12)CCCN3* ; *c1nc2c(*)cnn2c2c1CCN2]

Embodiment 28: The compound of any one of Embodiments 1-27, wherein m is 1 or 2.

Embodiment 29: The compound of any one of Embodiments 1-28, wherein m is 1.

Embodiment 30: The compound of any one of Embodiments 1-29, wherein R¹ is halogen.

Embodiment 31: The compound of any one of Embodiments 1-29, wherein R¹ is hydroxyl.

Embodiment 32: The compound of any one of Embodiments 1-29, wherein R¹ is cyano.

Embodiment 33: The compound of any one of Embodiments 1-29, wherein R¹ is C1-C6 alkyl.

Embodiment 34: The compound of any one of Embodiments 1-29, wherein R¹ is C1-C6 alkoxy.

Embodiment 35: The compound of any one of Embodiments 1-29, wherein R¹ is —C(═O)OR^{A}.

Embodiment 36: The compound of Embodiment 35, wherein R¹ is —C(═O)OH, —C(═O)OCH₂CH₃, or —C(═O)OC(CH₃)₃.

Embodiment 37: The compound of any one of Embodiments 1-29, wherein R¹ is —NR^{B}R^{C}.

Embodiment 38: The compound of any one of Embodiments 1-29, wherein R¹ is —C(═O)NR^{B}R^{C}.

Embodiment 39: The compound of Embodiment 38, wherein R¹ is —C(═O)NH₂ or —C(═O)NHCH₃.

Embodiment 40: The compound of any one of Embodiments 1-28, wherein m is 2.

Embodiment 41: The compound of any one of Embodiments 1-28 or 40, wherein one of R¹ is C1-C6 alkyl and the other R¹ is —C(═O)OR^{A} (e.g., —C(═O)OH or —C(═O)OCH₃).

Embodiment 42: The compound of any one of Embodiments 1-28 or 40, wherein one of R¹ is cyano.

Embodiment 43: The compound of Embodiment 42, wherein the other R¹ is halo.

Embodiment 44: The compound of Embodiment 42, wherein the other R¹ is hydroxy.

Embodiment 45: The compound of Embodiment 42, wherein the other R¹ is C1-C6 alkoxy.

Embodiment 46: The compound of any one of Embodiments 1-45, wherein n is 0.

Embodiment 47: The compound of any one of Embodiments 1-45, wherein n is 1, 2, or 3.

Embodiment 48: The compound of any one of Embodiments 1-45 or 47, wherein n is 1.

Embodiment 49: The compound of Embodiment 48, wherein R² is —C(═O)OR^{D}.

Embodiment 50: The compound of Embodiment 58 or 49, wherein R² is —C(═O)OCH(CH₃)₃.

Embodiment 51: The compound of Embodiment 48, wherein R² is C1-C6 alkyl.

Embodiment 52: The compound of Embodiment 48, wherein R² is C2-C6 alkynyl optionally substituted with 4-8 membered heterocyclyl optionally substituted with C1-C6 alkyl.

Embodiment 53: The compound of Embodiment 52, wherein R² is

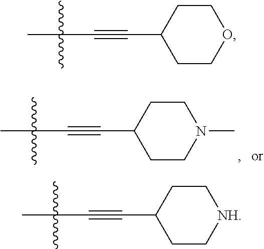

Embodiment 54: The compound of Embodiment 48, wherein R² is —C(═O)-phenyl.

Embodiment 55: The compound of Embodiment 48, wherein R² is —(C1-C6 alkyl)-phenyl.

Embodiment 56: The compound of Embodiment 55, wherein R² is

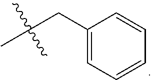 [SMILES: *Cc1ccccc1]

Embodiment 57: The compound of Embodiment 48, wherein R² is —(C1-C6 alkyl)-4-10 membered heterocyclyl optionally substituted with C1-C6 alkyl.

Embodiment 58: The compound of Embodiment 57, wherein R² is

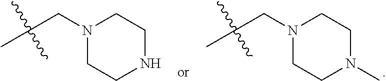

Embodiment 59: The compound of Embodiment 48, wherein R² is 4-10 membered heterocyclyl optionally substituted with C1-C6 alkyl or —CO₂C1-C6 alkyl.

Embodiment 60: The compound of Embodiment 59, wherein R² is

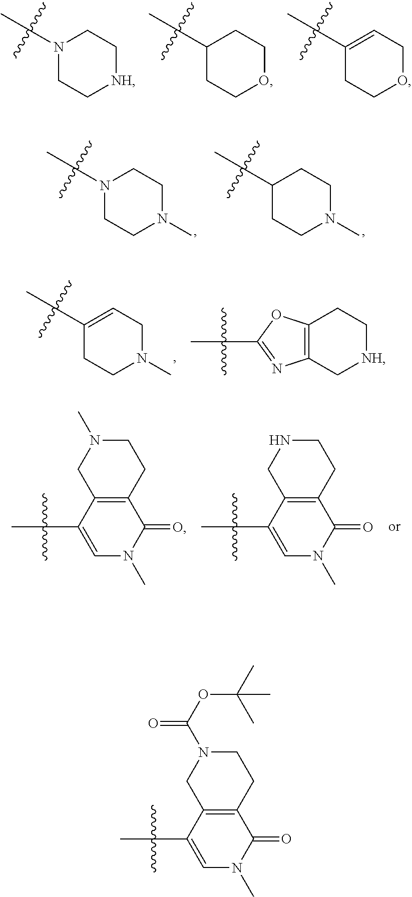

Embodiment 61: The compound of Embodiment 48, wherein R² is phenyl optionally substituted with cyano or fluoro.

Embodiment 62: The compound of Embodiment 61, wherein R² is

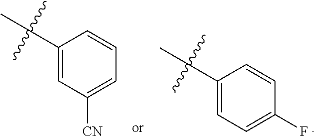 [SMILES: *c1cccc(C#N)c1 ; *c1ccc(F)cc1]

Embodiment 63: The compound of Embodiment 48, wherein R² is —NHC(═O)R^{E}.

Embodiment 64: The compound of Embodiment 63, wherein R² is

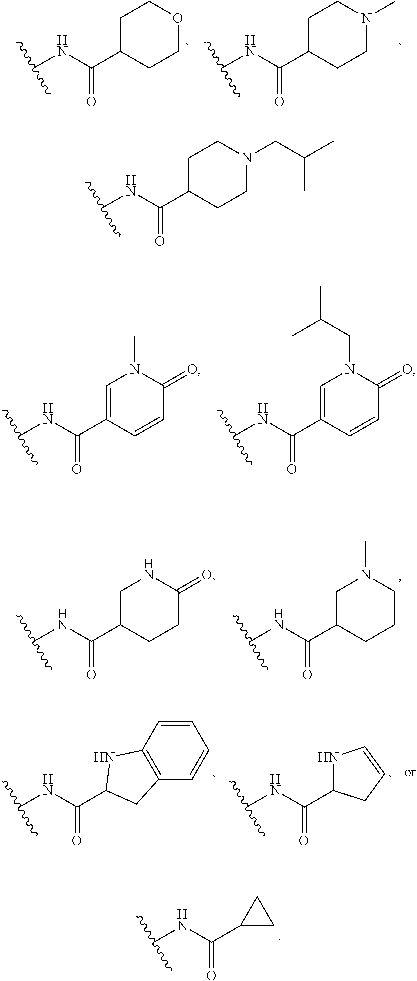

Embodiment 65: The compound of Embodiment 48, wherein R² is 5-6 membered heteroaryl optionally substituted with C1-C6 alkoxy.

Embodiment 66: The compound of Embodiment 65, wherein R² is

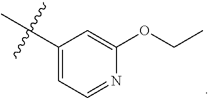 [SMILES: *c1ccnc(OCC)c1]

Embodiment 67: The compound of Embodiment 1, wherein the compound is a compound of formula (II-1):

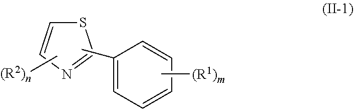

Embodiment 68: The compound of Embodiment 1, wherein the compound is a compound of formula (II-2):

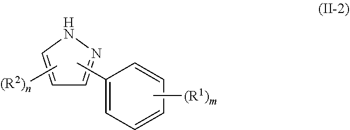

or a pharmaceutically acceptable salt thereof.

Embodiment 69: The compound of Embodiment 1, wherein the compound is a compound of formula (II-3):

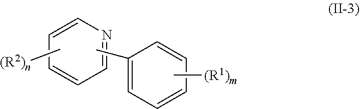

or a pharmaceutically acceptable salt thereof.

Embodiment 70: The compound of Embodiment 1, wherein the compound is a compound of formula (II-4):

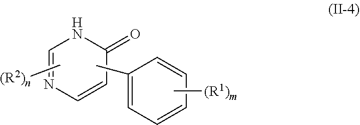

Embodiment 71: The compound of Embodiment 1, wherein the compound is a compound of formula (II-5):

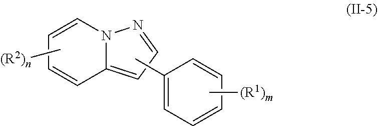

or a pharmaceutically acceptable salt thereof.

Embodiment 72: The compound of Embodiment 1, wherein the compound is a compound of formula (II-6):

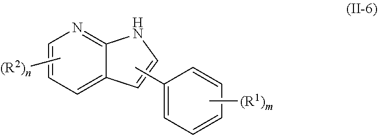

or a pharmaceutically acceptable salt thereof.

Embodiment 73: The compound of Embodiment 1, wherein the compound is a compound of formula (II-7):

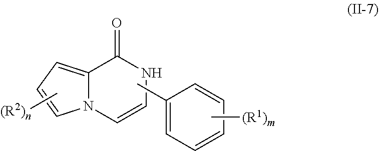

or a pharmaceutically acceptable salt thereof.

Embodiment 74: The compound of Embodiment 1, wherein the compound is a compound of formula (II-8):

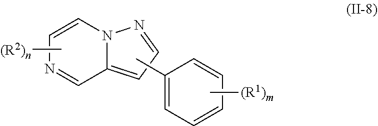

or a pharmaceutically acceptable salt thereof.

Embodiment 75: The compound of Embodiment 1, wherein the compound is a compound of formula (II-9):

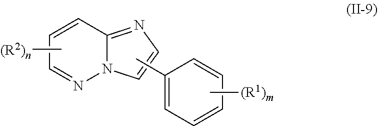

Embodiment 76: The compound of Embodiment 1, wherein the compound is a compound of formula (II-10):

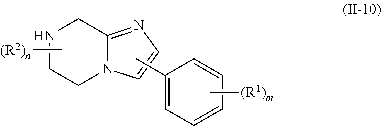

or a pharmaceutically acceptable salt thereof.

Embodiment 77: The compound of Embodiment 1, wherein the compound is a compound of formula (II-11):

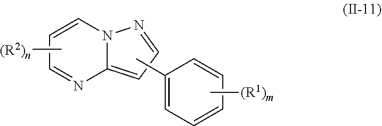

or a pharmaceutically acceptable salt thereof.

Embodiment 78: The compound of Embodiment 1, wherein the compound is a compound of formula (II-12):

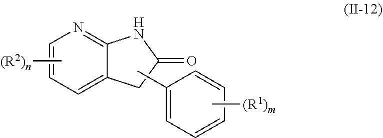

or a pharmaceutically acceptable salt thereof.

Embodiment 79: The compound of Embodiment 1, wherein the compound is a compound of formula (II-13):

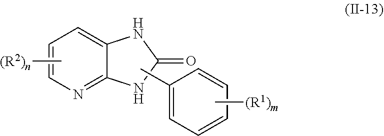

or a pharmaceutically acceptable salt thereof.

Embodiment 80: The compound of Embodiment 1, wherein the compound is a compound of formula (II-14):

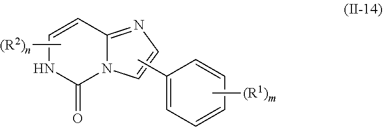

or a pharmaceutically acceptable salt thereof.

Embodiment 81: The compound of Embodiment 1, wherein the compound is a compound of formula (I-I15):

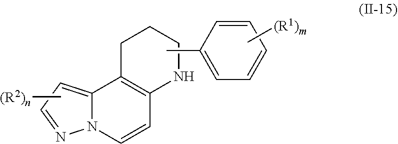

or a pharmaceutically acceptable salt thereof.

Embodiment 82: The compound of Embodiment 1, wherein the compound is a compound of formula (II-16):

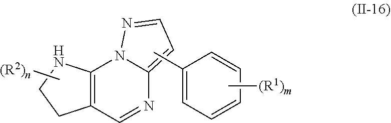

or a pharmaceutically acceptable salt thereof.

Embodiment 83: The compound of Embodiment 1, wherein the compound is a compound of formula (II-17):

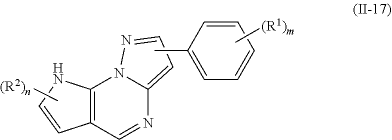

or a pharmaceutically acceptable salt thereof.

### Exemplary Embodiments of Compounds of Formula (III)

Embodiment 1: A compound of Formula (III):

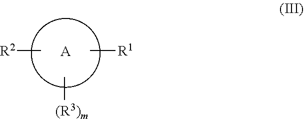

or a pharmaceutically acceptable salt thereof:
Ring A is 5-6 membered heteroaryl or 5-6 membered heterocyclyl;
R¹ is —NHC(═O)(C1-C6 alkylene)ₙR^{A}, phenyl optionally substituted with —NR^{F}R^{G}, -Q-R^{C}, or

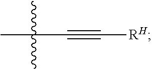

R² is C3-C6 cycloalkyl optionally substituted with —CO₂R^{B}, 5-10 membered heteroaryloxy, —(C1-C6 alkylene)ₚ-5-10 membered heteroaryl optionally substituted with C1-C6 alkyl, cyano, or 4-6 membered heterocyclyl; —(C1-C6 alkylene) t-phenyl optionally substituted with cyano or —NR^{D}R^{E}; 4-6 membered heterocyclyl optionally substituted with C1-C6 alkyl;
R³ is C1-C6 alkyl;
R^{A} is 4-6 membered heterocyclyl optionally substituted with C1-C6 alkyl, or 5-10 membered heteroaryl optionally substituted with C1-C6 alkoxy or C1-C6 alkyl,
R^{B} is hydrogen or C1-C6 alkyl;
R^{C} is 4-10 membered heterocyclyl, 5-10 membered heteroaryl, or phenyl optionally substituted with —(C1-C6 alkylene)-NR^{D}R^{E};
R^{D}, R^{E}, and R^{F} are independently hydrogen, C1-C6 alkyl, or C3-C6 cycloalkyl;
R^{G} is hydrogen, C1-C6 alkyl, —C(═O)—C1-C6 alkyl, or —C(═O)—C3-C6 cycloalkyl;
R^{H} is 4-6 membered heterocyclyl optionally substituted with 1-2 independently selected C1-C6 alkyl;
Q is C1-C6 alkylene, NH, or O;
m is 0 or 1;
n is 0 or 1;
p is 0 or 1; and
t is 0 or 1.

Embodiment 2: The compound of Embodiment 1, wherein R¹ is —NHC(═O)(C1-C6 alkylene)ₙR^{A}.

Embodiment 3: The compound of Embodiment 1 or 2, wherein R¹ is —NHC(═O)(C1-C2 alkylene)ₙR^{A}.

Embodiment 4: The compound of any one of Embodiments 1-3, wherein n is 1.

Embodiment 5: The compound of any one of Embodiments 1-3, wherein n is 0.

Embodiment 6: The compound of any one of Embodiments 1-5, wherein R^{A} is 4-6 membered heterocyclyl optionally substituted with C1-C6 alkyl.

Embodiment 7: The compound of any one of Embodiments 1-6, wherein R^{A} is oxetanyl, azetidinyl, pyrrolidinyl, pyrrolidinonyl, imidazolidinyl, piperidinyl, piperazinyl, or morpholinyl; each optionally substituted with C1-C6 alkyl.

Embodiment 8: The compound of any one of Embodiments 1-7, wherein R^{A} is 4-6 membered heterocyclyl substituted with C1-C6 alkyl.

Embodiment 9: The compound of any one of Embodiments 1-7, wherein R^{A} is unsubstituted 4-6 membered heterocyclyl.

Embodiment 10: The compound of any one of Embodiments 1-5, wherein R^{A} is 5-10 membered heteroaryl optionally substituted with C1-C6 alkoxy or C1-C6 alkyl.

Embodiment 11: The compound of any one of Embodiments 1-5 or 10, wherein R^{A} is 5-10 membered heteroaryl substituted with C1-C6 alkoxy.

Embodiment 12: The compound of any one of Embodiments 1-5 or 10, wherein R^{A} is 5-10 membered heteroaryl substituted with C1-C6 alkyl.

Embodiment 13: The compound of any one of Embodiments 1-5 or 10, wherein R^{A} is 5-6 membered heteroaryl optionally substituted with C1-C6 alkoxy or C1-C6 alkyl.

Embodiment 14: The compound of any one of Embodiments 1-5, 10, or 13, wherein R^{A} is pyrrolyl, imidazolyl, oxazolyl, thiazolyl, pyridinyl, pyrimidinyl, or pyrazinyl; each optionally substituted with C1-C6 alkoxy or C1-C6 alkyl.

Embodiment 15: The compound of any one of Embodiments 1-5, 10, or 13-14, wherein R^{A} is 5-6 membered heteroaryl substituted with C1-C6 alkoxy.

Embodiment 16: The compound of any one of Embodiments 1-5, 10, or 13-14, wherein R^{A} is 5-6 membered heteroaryl substituted with C1-C6 alkyl.

Embodiment 17: The compound of any one of Embodiments 1-5, 10, or 13-14, wherein R^{A} is unsubstituted 5-6 membered heteroaryl.

Embodiment 18: The compound of Embodiment 1, wherein R¹ is phenyl optionally substituted with —NR^{F}R^{G}.

Embodiment 19: The compound of Embodiment 1, wherein R¹ is phenyl substituted with —NR^{F}R^{G}.

Embodiment 20: The compound of Embodiment 1 or 19, wherein R^{F} is C1-C6 alkyl.

Embodiment 21: The compound of any one of Embodiments 1 or 19-20, wherein R^{F} is methyl.

Embodiment 22: The compound of Embodiment 1 or 19, wherein R^{F} is C3-C6 cycloalkyl.

Embodiment 23: The compound of Embodiment 1 or 19, wherein R^{F} is hydrogen.

Embodiment 24: The compound of any one of Embodiments 1 or 19-23, wherein R^{G} is C1-C6 alkyl.

Embodiment 25: The compound of any one of Embodiments 1 or 19-24, wherein R^{G} is methyl.

Embodiment 26: The compound of any one of Embodiments 1 or 19-23, wherein R^{G} is —C(═O)—C1-C6 alkyl.

Embodiment 27: The compound of any one of Embodiments 1, 19-23, or 26, wherein R^{G} is —C(═O)CH₃.

Embodiment 28: The compound of any one of Embodiments 1 or 19-23, wherein R^{G} is —C(═O)—C3-C6 cycloalkyl.

Embodiment 29: The compound of any one of Embodiments 1 or 19-23, wherein R^{G} is hydrogen.

Embodiment 30: The compound of Embodiment 1 or 19, wherein R^{F} and R^{G} are the same.

Embodiment 31: The compound of Embodiment 1 or 19, wherein R^{F} and R^{G} are different.

Embodiment 32: The compound of Embodiment 1 or 19, wherein R^{F} and R^{G} are each hydrogen.

Embodiment 33: The compound of Embodiment 1 or 19, wherein R^{F} and R^{G} are each methyl.

Embodiment 34: The compound of Embodiment 1 or 19, wherein R^{F} is hydrogen and R^{G} is C1-C6 alkyl.

Embodiment 35: The compound of Embodiment 1 or 19, wherein R^{F} is hydrogen and R^{G} is —C(═O)—C1-C6 alkyl.

Embodiment 36: The compound of Embodiment 1, wherein R¹ is -Q-R^{C}.

Embodiment 37: The compound of Embodiment 1 or 36, wherein Q is C1-C6 alkylene.

Embodiment 38: The compound of any one of Embodiments 1 or 36-37, wherein Q is C1-C2 alkylene.

Embodiment 39: The compound of any one of Embodiments 1 or 36-38, wherein Q is methylene.

Embodiment 40: The compound of Embodiment 1 or 36, wherein Q is NH.

Embodiment 41: The compound of Embodiment 1 or 36, wherein Q is O.

Embodiment 42: The compound of Embodiment 1, wherein R¹ is

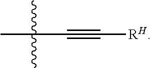

Embodiment 43: The compound of Embodiment 1 or 42, wherein R^{H} is 4-6 membered heterocyclyl substituted with 1-2 independently selected C1-C6 alkyl.

Embodiment 44: The compound of any one of Embodiments 1 or 42-43, wherein R^{H} is 4-6 membered heterocyclyl substituted with one C1-C6 alkyl.

Embodiment 45: The compound of any one of Embodiments 1 or 42-44, wherein R^{H} is 4-6 membered heterocyclyl substituted with methyl.

Embodiment 46: The compound of any one of Embodiments 1 or 42-43, wherein R^{H} is 4-6 membered heterocyclyl substituted with two independently selected C1-C6 alkyl.

Embodiment 47: The compound of any one of Embodiments 1, 42-43, or 46, wherein R^{H} is 4-6 membered heterocyclyl substituted with two methyls.

Embodiment 48: The compound of any one of Embodiment 1 or 42, wherein R^{H} is oxetanyl, azetidinyl, pyrrolidinyl, pyrrolidinonyl, imidazolidinyl, piperidinyl, piperazinyl, or morpholinyl; each optionally substituted with 1-2 independently selected C1-C6 alkyl.

Embodiment 49: The compound of any one of Embodiments 1-48, wherein R² is C3-C6 cycloalkyl optionally substituted with —CO₂R^{B}.

Embodiment 50: The compound of any one of Embodiments 1-49, wherein R² is C3-C6 cycloalkyl substituted with —CO₂R^{B}.

Embodiment 51: The compound of any one of Embodiments 1-50, wherein R^{B} is C1-C6 alkyl.

Embodiment 52: The compound of any one of Embodiments 1-51, wherein R^{B} is methyl.

Embodiment 53: The compound of any one of Embodiments 1-50, wherein R^{B} is hydrogen.

Embodiment 54: The compound of any one of Embodiments 1-49, wherein R² is unsubstituted C3-C6 cycloalkyl.

Embodiment 55: The compound of any one of Embodiments 1-48, wherein R² is 5-10 membered heteroaryloxy.

Embodiment 56: The compound of any one of Embodiments 1-48 or 55, wherein R² is 9-10 membered heteroaryloxy.

Embodiment 57: The compound of any one of Embodiments 1-48 or 55, wherein R² is 5-6 membered heteroaryloxy.

Embodiment 58: The compound of any one of Embodiments 1-48, wherein R² is —(C1-C6 alkylene)ₚ-5-10 membered heteroaryl optionally substituted with C1-C6 alkyl, cyano, or 4-6 membered heterocyclyl.

Embodiment 59: The compound of any one of Embodiments 1-48 or 58, wherein R² is —(C1-C6 alkylene)ₚ-5-10 membered heteroaryl substituted with C1-C6 alkyl, cyano, or 4-6 membered heterocyclyl.

Embodiment 60: The compound of any one of Embodiments 1-48 or 58-59, wherein R² is —(C1-C6 alkylene)ₚ-5-10 membered heteroaryl substituted with C1-C6 alkyl.

Embodiment 61: The compound of any one of Embodiments 1-48 or 58-60, wherein R² is —(C1-C6 alkylene)ₚ-5-10 membered heteroaryl substituted with methyl.

Embodiment 62: The compound of any one of Embodiments 1-48 or 58-59, wherein R² is —(C1-C6 alkylene)ₚ-5-10 membered heteroaryl substituted with cyano.

Embodiment 63: The compound of any one of Embodiments 1-48 or 58-59, wherein R² is —(C1-C6 alkylene)ₚ-5-10 membered heteroaryl substituted with 4-6 membered heterocyclyl.

Embodiment 64: The compound of any one of Embodiments 1-48, 58-59, or 63, wherein R² is —(C1-C6 alkylene)ₚ-5-10 membered heteroaryl substituted with oxetanyl, azetidinyl, pyrrolidinyl, pyrrolidinonyl, imidazolidinyl, piperidinyl, piperazinyl, or morpholinyl.

Embodiment 65: The compound of any one of Embodiments 1-48 or 58, wherein R² is unsubstituted —(C1-C6 alkylene)ₚ-5-10 membered heteroaryl.

Embodiment 66: The compound of any one of Embodiments 1-48 or 58-65, wherein p is 1.

Embodiment 67: The compound of any one of Embodiments 1-48 or 58-65, wherein p is 0.

Embodiment 68: The compound of any one of Embodiments 1-48, wherein R² is —(C1-C6 alkylene) t-phenyl optionally substituted with cyano or —NR^{D}R^{E}.

Embodiment 69: The compound of any one of Embodiments 1-48 or 68, wherein R² is —(C1-C6 alkylene) t-phenyl substituted with cyano or —NR^{D}R^{E}.

Embodiment 70: The compound of any one of Embodiments 1-48 or 68-69, wherein R² is —(C1-C6 alkylene) t-phenyl substituted with —NR^{D}R^{E}.

Embodiment 71: The compound of any one of Embodiments 1-48 or 68-70, wherein R^{D} is C1-C6 alkyl.

Embodiment 72: The compound of any one of Embodiments 1-48 or 68-71, wherein R^{D} is methyl.

Embodiment 73: The compound of any one of Embodiments 1-48 or 68-70, wherein R^{D} is C3-C6 cycloalkyl.

Embodiment 74: The compound of any one of Embodiments 1-48 or 68-70, wherein R^{D} is hydrogen.

Embodiment 75: The compound of any one of Embodiments 1-48 or 68-74, wherein R^{E} is C1-C6 alkyl.

Embodiment 76: The compound of any one of Embodiments 1-48 or 68-75, wherein R^{E} is methyl.

Embodiment 77: The compound of any one of Embodiments 1-48 or 68-74, wherein R^{E} is C3-C6 cycloalkyl.

Embodiment 78: The compound of any one of Embodiments 1-48 or 68-74, wherein R^{E} is hydrogen.

Embodiment 79: The compound of any one of Embodiments 1-48 or 68-78, wherein t is 1.

Embodiment 80: The compound of any one of Embodiments 1-48 or 68-78, wherein t is 0.

Embodiment 81: The compound of any one of Embodiments 1-48, wherein R² is 4-6 membered heterocyclyl optionally substituted with C1-C6 alkyl.

Embodiment 82: The compound of any one of Embodiments 1-48 or 81, wherein R² is 4-6 membered heterocyclyl substituted with C1-C6 alkyl.

Embodiment 83: The compound of any one of Embodiments 1-48 or 81-82, wherein R² is 4-6 membered heterocyclyl substituted with methyl.

Embodiment 84: The compound of any one of Embodiments 1-48 or 81, wherein R² is oxetanyl, azetidinyl, pyrrolidinyl, pyrrolidinonyl, imidazolidinyl, piperidinyl, piperazinyl, or morpholinyl; each optionally substituted with C1-C6 alkyl.

Embodiment 85: The compound of any one of Embodiments 1-84, wherein R³ is C1-C3 alkyl.

Embodiment 86: The compound of any one of Embodiments 1-85, wherein R³ is methyl.

Embodiment 87: The compound of any one of Embodiments 1-86, wherein R^{C} is 4-10 membered heterocyclyl.

Embodiment 88: The compound of any one of Embodiments 1-87, wherein R^{C} is 4-6 membered heterocyclyl.

Embodiment 89: The compound of any one of Embodiments 1-86, wherein R^{C} is 5-10 membered heteroaryl.

Embodiment 90: The compound of any one of Embodiments 1-86 or 89, wherein R^{C} is 5-6 membered heteroaryl.

Embodiment 91: The compound of any one of Embodiments 1-86, wherein R^{C} is phenyl optionally substituted with —(C1-C6 alkylene)-NR^{D}R^{E}.

Embodiment 92: The compound of any one of Embodiments 1-86 or 91, wherein R^{C} is phenyl substituted with —(C1-C6 alkylene)-NR^{D}R^{E}.

Embodiment 93: The compound of any one of Embodiments 1-86 or 91-92, wherein R^{C} is phenyl substituted with —(C1-C2 alkylene)-NR^{D}R^{E}.

Embodiment 94: The compound of any one of Embodiments 91-93, wherein R^{D} is C1-C6 alkyl.

Embodiment 95: The compound of any one of Embodiments 91-94, wherein R^{D} is methyl.

Embodiment 96: The compound of any one of Embodiments 91-93, wherein R^{D} is C3-C6 cycloalkyl.

Embodiment 97: The compound of any one of Embodiments 91-93, wherein R^{D} is hydrogen.

Embodiment 98: The compound of any one of Embodiments 91-97, wherein R^{E} is C1-C6 alkyl.

Embodiment 99: The compound of any one of Embodiments 91-98, wherein R^{E} is methyl.

Embodiment 100: The compound of any one of Embodiments 91-97, wherein R^{E} is C3-C6 cycloalkyl.

Embodiment 101: The compound of any one of Embodiments 91-97, wherein R^{E} is hydrogen.

Embodiment 102: The compound of any one of Embodiments 1-86 or 91, wherein R^{C} is unsubstituted phenyl.

Embodiment 103: The compound of any one of Embodiments 1-102, wherein m is 1.

Embodiment 104: The compound of any one of Embodiments 1-102, wherein m is 0.

Embodiment 105: The compound of any one of Embodiments 1-102, wherein Ring A is 5-6 membered heteroaryl.

Embodiment 106: The compound of any one of Embodiments 1-102, wherein Ring A is thiazolyl, pyrazolyl, imidazolidinon-2-yl, pyridinyl, pyrimidinyl, pyridon-2-yl, pyrimidinonyl, or oxazolidinon-2-yl.

Embodiment 107: The compound of any one of Embodiments 1-102 or 105, wherein Ring

A is

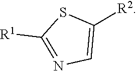

Embodiment 108: The compound of any one of Embodiments 1-102 or 105, wherein Ring A is

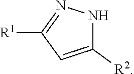

Embodiment 109: The compound of any one of Embodiments 1-102 or 105, wherein Ring A is

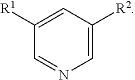

Embodiment 110: The compound of any one of Embodiments 1-102 or 105, wherein Ring A is

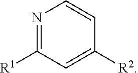

Embodiment 111: The compound of any one of Embodiments 1-102 or 105, wherein Ring A is

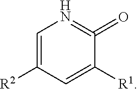

Embodiment 112: The compound of any one of Embodiments 1-102 or 105, wherein Ring A is

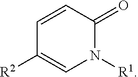

Embodiment 113: The compound of any one of Embodiments 1-102 or 105, wherein Ring A is

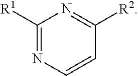

Embodiment 114: The compound of any one of Embodiments 1-102, wherein Ring A is 5-6 membered heterocyclyl.

Embodiment 115: The compound of any one of Embodiments 1-102 or 114, wherein Ring A is oxazolidinone or pyrrolidinone.

Embodiment 116: The compound of any one of Embodiments 1-102 or 114, wherein Ring A is

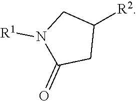

Embodiment 117: The compound of any one of Embodiments 1-102 or 114, wherein Ring A is

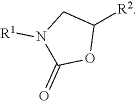

### Further Exemplary Embodiments of Compounds of Formulae (I), (II), and (III)

Embodiment 1: A compound, or a pharmaceutically acceptable salt thereof, selected from a compound in Table 1, Table 2, Table 3, or Table 4, or a pharmaceutically acceptable salt of any of the foregoing.

Embodiment 2: A pharmaceutical composition comprising a compound of Embodiment 1 or any one of the Embodiments of compounds of Formulae (I), (II), and (III), or a pharmaceutically acceptable salt thereof, and pharmaceutically acceptable diluent or carrier.

Embodiment 3: A method for treating a neurological disorder in a subject in need thereof, the method comprising administering to the subject a therapeutically effective amount of a compound of Embodiment 1 or any one of the Embodiments of compounds of Formulae (I), (II), and (III), or a pharmaceutically acceptable salt thereof, or a pharmaceutical composition according to Embodiment 2.

Embodiment 4: The method of Embodiment 3, wherein the neurological disorder is selected from the group consisting of Down Syndrome, Alzheimer's disease, and Alzheimer's disease associated with Down Syndrome.

Embodiment 5: The method of Embodiment 3 or 4, wherein the neurological disorder is selected Alzheimer's disease associated with Down syndrome.

Embodiment 6: A method of treating a subject, the method comprising administering a therapeutically effective amount of a compound of Embodiment 1 or any one of the Embodiments of compounds of Formulae (I), (II), and (III), or a pharmaceutically acceptable salt thereof, or a pharmaceutical composition according to Embodiment 2, to a subject having a clinical record that indicates that the subject has a dysregulation of a DYRK1A gene, DYRK1A protein, or expression or activity or level of any of the same.

Embodiment 7: A method of treating a DYRK1A-associated neurological disorder in a subject, the method comprising administering to a subject identified or diagnosed as having a DYRK1A-associated neurological disorder a therapeutically effective amount of a compound of Embodiment 1 or any one of the Embodiments of compounds of Formulae (I), (II), and (III), or a pharmaceutically acceptable salt thereof, or a pharmaceutical composition according to Embodiment 2.

Embodiment 8: A method of treating a DYRK1A-associated neurological disorder in a subject, the method comprising:
determining that the neurological disorder in the subject is a DYRK1A-associated neurological disorder; and
administering to the subject a therapeutically effective amount of a compound of any one of Embodiment 1 or any one of the Embodiments of compounds of Formulae (I), (II), and (III), or a pharmaceutically acceptable salt thereof, or a pharmaceutical composition according to Embodiment 2.

Embodiment 9: A method for treating a neurological disorder in a subject in need thereof, the method comprising (a) determining that the neurological disorder is associated with a dysregulation of a DYRK1A gene, a DYRK1A protein, or expression or activity or level of any of the same; and (b) administering to the subject a therapeutically effective amount of a compound of Embodiment 1 or any one of the Embodiments of compounds of Formulae (I), (II), and (III), or a pharmaceutically acceptable salt thereof, or a pharmaceutical composition according to Embodiment 2.

Embodiment 10: The method of Embodiment 8 or 9, wherein the step of determining that the neurological disorder in the subject is a DYRK1A-associated neurological disorder includes performing an assay to detect dysregulation in a DYRK1A gene, a DYRK1A protein, or expression or activity or level of any of the same in a sample from the subject.

Embodiment 11: A method for treating a neurological disorder in a subject in need thereof, the method comprising (a) determining that the neurological disorder is associated with Down Syndrome; and (b) administering to the subject a therapeutically effective amount of a compound of Embodiment 1 or any one of the Embodiments of compounds of Formulae (I), (II), and (III), or a pharmaceutically acceptable salt thereof, or a pharmaceutical composition according to Embodiment 2.

Embodiment 12: The method of Embodiment 11, wherein the step of determining that the neurological disorder in the subject is associated with Down Syndrome includes performing an assay on a sample from the subject.

Embodiment 13: The method of any one of Embodiments 8-12, wherein the assay is selected from the group consisting of sequencing, immunohistochemistry, enzyme-linked immunosorbent assay, and fluorescence in situ hybridization (FISH).

Embodiment 14: The method of any one of Embodiments 7, 8, or 305, wherein the DYRK1A-associated neurological disorder is selected from the group consisting of Down Syndrome, Alzheimer's disease, and Alzheimer's disease associated with Down Syndrome.

Embodiment 15: The method of any one of Embodiments 7, 8, 305, or 309, wherein the DYRK1A-associated neurological disorder is Alzheimer's disease associated with Down syndrome.

Embodiment 16: A method for modulating DYRK1A in a mammalian cell, the method comprising contacting the mammalian cell with a therapeutically effective amount of a compound of Embodiment 1 or any one of the Embodiments of compounds of Formulae (I), (II), and (III), or a pharmaceutically acceptable salt thereof.

Embodiment 17: The method of Embodiment 16, wherein the contacting occurs in vivo.

Embodiment 18: The method of Embodiment 16, wherein the contacting occurs in vitro.

Embodiment 19: The method of any one of Embodiments 16-18, wherein the mammalian cell is a mammalian neural cell.

Embodiment 20: The method of Embodiment 19, wherein the mammalian neural cell is a mammalian DYRK1A-associated neural cell.

Embodiment 21: The method of any one of Embodiments 16-20, wherein the cell has a dysregulation of a DYRK1A gene, a DYRK1A protein, or expression or activity or level of any of the same.

Embodiment 22: The method of any one of Embodiments 16-21, wherein the cell has a chromosomal abnormality associated with Down Syndrome.

### EXAMPLES

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following the schemes provided herein, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

### EXAMPLES

### General

¹H NMR spectra were recorded on Bruker Avance 400 MHz.

Unless otherwise indicated, LCMS was taken on a quadruple Mass Spectrometer on Shimadzu LCMS 2010 (Column: Shim-pack XR-ODS (3.0×30 mm, 2.2 m)) operating in ESI (+) ionization mode. Flow Rate: 0.8 mL/min, Acquire Time: 2 min or 3 min, Wavelength: UV220, Oven Temp.: 50° C.

Prep-HPLC was performed at conditions:

Method A: Column: Fuji C18 (300×25), YMC 250×20; Wavelength: 220 nm; Mobile phase: A CH₃CN (0.05% FA); B water (0.05% FA); Flow rate: 25 mL/min; Injection Volume: 2 mL; Run time: 20 min; Equilibration: 3 min.

Method B: Column: Fuji C18 (300×25), YMC 250×20; Wavelength: 220 nm; Mobile phase: A CH₃CN (0.05% NH₃·H₂O as an additive); B water (0.05% NH₃·H₂O as an additive); Flow rate: 25 mL/min; Injection Volume: 2 mL; Run time: 20 min; Equilibration: 3 min.

Method C: Column: Phenomenex luna C18 (100×25), YMC (250×20); Wavelength: 220 nm; Mobile phase: A CH₃CN; B water (0.225% FA); Flow rate: 25 mL/min; Injection Volume: 2 mL; Run time: 10 min; Equilibration: 3 min.

Method D: Column: Fuji C18 (300×25), YMC 250×20; Wavelength: 220 nm; Mobile phase: A CH₃CN (0.05% HCOONH₄ as an additive); B water (0.05% HCOONH₄ as an additive); Flow rate: 25 mL/min; Injection Volume: 2 mL; Run time: 20 min; Equilibration: 3 min.

Method E: Prep-HPLC (0.04% NH₃H₂O+10 mM NH₄HCO₃

Method F: Phenomenex luna C18 (100×25), YMC (250×20); Wavelength: 220 nm; Mobile phase: A CH₃CN; B water (0.04% HCl); Flow rate: 25 mL/min; Injection Volume: 2 mL; Run time: 10 min; Equilibration: 3 min.

### Abbreviations

The following abbreviations have the indicated meanings:
Acac=acetylacetone
AcCl=acetyl chloride
ACE-Cl=1-Chloroethyl chloroformate
ACN=Acetonitrile
BINAP=(±)-2,2′-Bis(diphenylphosphino)-1,1′-binaphthalene
Bis-Pin=Bis(pinacolato)diboron
BnBr=(bromomethyl)benzene
BPO=Benzoyl Peroxide
Boc=t-butyloxy carbonyl
Boc₂O=di-tert-butyl dicarbonate
BocNH₂=tert-butyl carbamate
BrettPhos Pd G3=methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II)
BrettPhos=2-dicyclohexylphosphino-2′,4′,6′-triisopropyl-3,6-dimethoxybiphenyl
CbzCl=benzyl carbonochloridate
Cbz=carbobenzyloxy
CDI=1,1′-Carbonyldiimidazole
CuCN=Copper(I) cyanide
CuI=Copper (I) iodide
CPME=Cyclopentyl methyl ether
CyJohnphos=(2-Biphenyl)dicyclohexylphosphine
DAST=diethylaminosulfur trifluoride
DBU=1,8-diazabicyclo(5.4.0)undec-7-ene
DCE=1,2-dichloroethane
DCM=dichloromethane
DEAD=diethyl azodicarboxylate
DIEA=N, N-diisopropylethylamine
DIPEA=N, N-diisopropylethylamine
DIAD=diisopropyl azodicarboxylate
DME=dimethoxyethane
DMEDA=N,N′-Dimethylethylenediamine
DMF=N, N-dimethylformamide
DMAP=N, N-dimethylpyridin-4-amine
DMSO=dimethyl sulfoxide
Dioxane=1,4-dioxane
DPPA=Diphenylphosphoryl azide
DPPP=1,3-Bis(diphenylphosphino)propane
Dtbpy=4,4′-di-tert-butyl-2,2′dipyridine
EDCI=1-(3-dimethylaminopropyl)-3-ethylcarbodiimide hydrochloride
EtI=Ethyl iodide
EtOAc=ethyl acetate
FA=formic acid
Grubb's II catalyst=Benzylidene[1,3-bis(2,4,6-trimethylphenyl)-2-imidazolidinylidene]dichloro(tricyclohexylphosphine)rutheniumum
HATU=N, N, N, N-tetramethyl-o-(7-azabenzotriazol-1-yl)uronium hexafluorophosphate
HBPin=4,4,5,5-Tetramethyl-1,3,2-dioxaborolane
HF/Py=hydrogen fluoride-pyridine
HOAc=acetic acid
HOBt=1-hydroxybenzotriazole hydrate
HPLC=high performance liquid chromatography
[Ir(OMe)(COD)]₂=Bis(1,5-cyclooctadiene)di-μ-methoxydiiridium(I)
KOAc=potassium acetate
LAH/THF=lithium aluminium hydride, 1M solution in THF
LDA=Lithium diisopropylamide
LiHMDS=lithium bis(trimethylsilyl)amide
LC-MS=liquid chromatography-mass spectrometry
M-CPBA=3-Chloroperoxybenzoic acid
Me=methyl
MeOH=methanol
MeCN=acetonitrile
Me₂SO₄=Dimethyl sulfate
MsCl=Methanesulfonyl chloride
NaBH₃CN=Sodium cyanoborohydride
NaOAc=Sodium acetate
NBS=N-bromosuccinimide
n-BuLi=Butyl lithium
n-Bu3SnH=Tributyltin hydride
NCS=N-Chlorosuccinimide
NIS=N-iodosuccinimide
NMI=1-methylimidazole
NMO=N-Methylmorpholine N-oxide
NMP=N-methylpyrrolidone
NMR=nuclear magnetic resonance
PCy₃=tricyclohexyl phosphine
Pd₂(dba)₃-CHCl₃=tris(dibenzylideneacetone)dipalladium(O)-chloroform adduct
Pd(dppf)Cl₂—CH₂Cl₂=1,1′-bis(diphenylphosphino)ferrocene-palladium(II)dichloride dichloromethane complex
Pd/C=Palladium (0) on activated carbon
Pd(dtbpf)Cl₂=[1,1′-Bis(di-tert-butylphosphino)ferrocene]dichloropalladium(II)
Pd(dppf)Cl₂=[1,1′-bis(diphenylphosphino)ferrocene]dichloropalladium(II)
Pd(OAc)₂=Palladium(II) acetate
Pd(PCy₃)₂Cl₂=Dichlorobis(tricyclohexylphosphine)palladium(ii)
Pd(PPh₃)₂Cl₂=bis(triphenylphosphine)palladium chloride
Pd(PPh₃)₄=Tetrakis-(triphenylphosphine)-palladium
PE=petroleum ether
PPA=Polyphosphoric acid
PPh₃=Triphenylphosphine
PPTS=Pyridinium p-toluenesulfonate
PtO₂=Platinum(IV) Oxide
Pre-HPLC=preparative high performance liquid chromatography
RT=room temperature
Ruphos-Pd-G2=Chloro(2-dicyclohexylphosphino-2′,6′-diisopropoxy-1,1′-biphenyl)[2-(2′-amino-1,1′-biphenyl)]palladium(II)
Ruphos=2-Dicyclohexylphosphino-2′,6′-diisopropoxybiphenyl
Sat.=saturated
SEM-Cl=2-(trimethylsily)ethoxymethyl chloride
Sodium phosphate buffer=sodium phosphate
Speedvac=Savant SC250EXP SpeedVac Concentrator
Sulfolane=1λ⁶-Thiolane-1,1-dione
T₃P=propanephosphonic acid cyclic anhydride
Trt=Trityl protecting group
TABF=tetrabutylammonium fluoride trihydrate
TBAB=Tetrabutylammonium bromide
TBHP=tert-Butyl hydroperoxide
TBSCl=tert-butyldimethylsilyl chloride
TCFH=chloro-N,N,N,N-tetramethylformamidinium hexafluorophosphate
TMAD=N,N,N′,N′-Tetramethylazodicarboxamide
TMEDA=Tetramethylethylenediamine
TsCl=4-Toluenesulfonyl chloride
t-BuOK=potassium tert-butoxide
t-BuONO=tert-Butyl nitrite
t-Bu₃PHBF₄=Tri-tert-butylphosphonium tetrafluoroborate
TCFH=N, N, N, N-tetramethylchloroformamidinium hexafluorophosphate
TEA=trimethylamine
TEMPO=2,2,6,6-tetramethylpiperidinooxy
TFA=trifluoroacetic acid
TFAA=trifluoroacetic anhydride
THF=tetrahydrofuran
TLC=thin layer chromatography
TMAD=Tetramethylazodicarboxamide
TMSCHNH₂=Trimethylsilyldiazomethane
TMSCN=Trimethylsilyl cyanide;
UV=ultraviolet
XantPhos Pd G2=chloro[(4,5-bis(diphenylphosphino)-9,9-dimethylxanthene)-2-(2-amino-1,1-biphenyl)]palladium(II)
XantPhos=4,5-bis(diphenylphosphino)-9,9-dimethylxanthene
Xphos=2-dicyclohexylphosphino-2′,4′,6′-triisopropylbiphenyl
Xphos Pd G3=methanesulfonato(2-dicyclohexylphosphino-2′,4′,6′-tri-i-propyl-1,1′-biphenyl) (2′-amino-1,1′-biphenyl-2-yl)palladium(II) dichloromethane adduct
X³=three times
X²=two times

### Intermediates of Formula (I)

### Intermediate 1

### 6-(4,4,5,5-tetramethyl-1,3,2-dioxaborolan-2-yl)isoquinoline

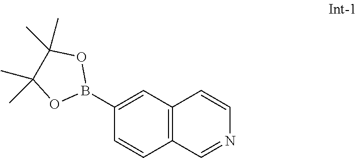 [SMILES: CC1(C)OB(c2ccc3cnccc3c2)OC1(C)C]

To a mixture of 6-bromoisoquinoline (500 mg, 2.40 mmol), Bis-pin (1.83 g, 7.21 mmol), KOAc (710 mg, 7.23 mmol) and Pd(dppf)Cl₂·CH₂Cl₂ (294 mg, 0.360 mmol) in anhydrous dioxane (6 mL) was degassed and purged with N₂ for 3 times. Then the reaction mixture was stirred at 80° C. for 2 hours under N₂ atmosphere. The mixture was concentrated to give 6-(4,4,5,5-tetramethyl-1,3,2-dioxaborolan-2-yl)isoquinoline (415 mg, crude) as a black solid, which was used directly for subsequent steps.

### Intermediate 2

### 6-bromo-4-iodoisoquinoline

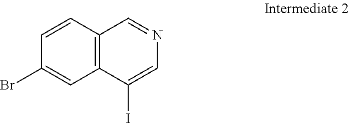 [SMILES: Brc1ccc2cncc(I)c2c1]

The mixture of 6-bromoisoquinoline (14.0 g, 67.3 mmol), 12 (34.2 g, 132 mmol) and TBHP (18.2 g, 202 mmol) in DCE (200 mL) was stirred at 120° C. for 24 hours. The reaction mixture was quenched by saturated solution of Na₂SO₃ (300 mL) at 20° C. and then diluted with H₂O (100 mL), extracted with DCM (300 mL×3). The combined organic layer was washed with brine (700 mL), dried over anhydrous Na₂SO₄, filtered and concentrated to give a residue. The residue was purified by flash silica gel chromatography (ISCO®; 120 g SepaFlash® Silica Flash Column, Eluent of 0˜22% Ethyl acetate/Petroleum ether gradient @75 mL/min) to give 6-bromo-4-iodoisoquinoline (19.0 g, yield: 85%) as a light yellow solid.

¹H NMR (400 MHz, CDCl₃) δ 7.73-7.83 (2H, m), 8.22 (1H, s), 8.97 (1H, s), 9.12 (1H, s).

### Intermediate 3

### 6-bromoisoquinolin-4-ol

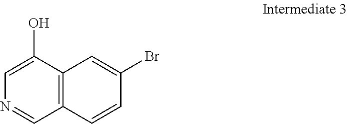 [SMILES: Oc1cncc2ccc(Br)cc12]

### Step 1. Synthesis of N-(4-bromobenzyl)-4-methylbenzenesulfonamide

To a solution of (4-bromophenyl)methanamine (10.0 g, 53.8 mmol) and 4-methylbenzenesulfonyl chloride (12.3 g, 64.5 mmol) in DCM (120 mL) was added Et₃N (16.3 g, 161 mmol), the mixture was stirred at 20° C. for 12 hours. The reaction mixture was quenched with saturated aqueous NaHCO₃ (120 mL) and separated. The aqueous phase was extracted with DCM (120 mL×2). The combined organic phases was washed with brine (200 mL), dried over anhydrous Na₂SO₄, filtered and concentrated. The residue was purified by flash silica gel chromatography (ISCO®; 80 g SepaFlash® Silica Flash Column, Eluent of 28˜42% Ethyl acetate/Petroleum ether gradient @60 mL/min) to give N-(4-bromobenzyl)-4-methylbenzenesulfonamide (16.5 g, yield: 90%) as a yellow solid.

¹H NMR (400 MHz, CDCl₃) δ 2.45 (3H, s), 4.09 (2H, d, J=6.4 Hz), 4.70 (1H, t, J=6.0 Hz), 7.06-7.12 (2H, m), 7.28-7.35 (2H, m), 7.38-7.43 (2H, m), 7.71-7.78 (2H, m).

### Step 2. Synthesis of ethyl N-(4-bromobenzyl)-N-tosylglycinate

To a solution of N-(4-bromobenzyl)-4-methylbenzenesulfonamide (15.4 g, 45.3 mmol) in THF (100 mL) was added NaH (1.90 g, 47.5 mmol, 60% dispersion in mineral oil) at 0° C., then the mixture was stirred at 20° C. for 2 hours. Ethyl 2-bromoacetate (11.3 g, 67.9 mmol) was added to above mixture at 0° C., the mixture was stirred at 20° C. for 3 hours. The reaction mixture was quenched with H₂O (200 mL) and extracted with DCM (200 mL×3). The combined organic layer was washed with brine (250 mL), dried over anhydrous Na₂SO₄, filtered and concentrated. The residue was purified by flash silica gel chromatography (ISCO®; 80 g SepaFlash® Silica Flash Column, Eluent of 14˜90% Ethyl acetate/Petroleum ether gradient @65 mL/min) to give ethyl N-(4-bromobenzyl)-N-tosylglycinate (18.9 g, yield: 98%) as a light yellow solid.

### Step 3. Synthesis of N-(4-bromobenzyl)-N-tosylglycine

To a solution of ethyl N-(4-bromobenzyl)-N-tosylglycinate (18.9 g, 44.3 mmol) in THE (150 mL), H₂O (75 mL) and MeOH (150 mL) was added LiOH·H₂O (5.58 g, 133 mmol), the mixture was stirred at 20° C. for 12 hours. The reaction mixture was concentrated and the residue was diluted into H₂O (260 mL), then acidified with 1N aqueous HCl to pH=2 and extracted with DCM (230 mL×3). The combined organic layer was dried over anhydrous Na₂SO₄, filtered and concentrated to give N-(4-bromobenzyl)-N-tosylglycine (17.5 g, crude) as white solid, which was used into the next step without further purification.

### Step 4. Synthesis of N-(4-bromobenzyl)-N-tosylglycinoyl chloride

A solution of N-(4-bromobenzyl)-N-tosylglycine (17.5 g, 43.9 mmol) in SOCl₂ (100 mL) was stirred at 80° C. for 2 hours. The reaction mixture was concentrated to give N-(4-bromobenzyl)-N-tosylglycinoyl chloride (18.5 g, crude) as a light yellow solid, which was used into the next step without further purification.

### Step 5. Synthesis of 6-bromo-2-tosyl-2,3-dihydroisoquinolin-4(1H)-one

To a solution of N-(4-bromobenzyl)-N-tosylglycinoyl chloride (18.5 g, 44.4 mmol) in DCM (600 mL) was added AlCl₃ (23.7 g, 178 mmol) at 0° C. in small portions, the mixture was stirred at 20° C. for 3 hours under N₂ atmosphere. The reaction mixture was quenched with aqueous 2N aqueous NaOH to pH=14 and diluted with H₂O (400 mL), then separated. The aqueous phase was extracted with DCM (400 mL×2), the combined organic layer was dried over anhydrous Na₂SO₄, filtered and concentrated. The residue was purified by flash silica gel chromatography (ISCO®; 80 g SepaFlash® Silica Flash Column, Eluent of 22-45% Ethyl acetate/Petroleum ether gradient @60 mL/min) to give 6-bromo-2-tosyl-2,3-dihydroisoquinolin-4(1H)-one (9.00 g, yield: 47% for three steps) as a light yellow solid.

### Step 6. Synthesis of 6-bromoisoquinolin-4-ol

To a solution of 6-bromo-2-tosyl-2,3-dihydroisoquinolin-4(1H)-one (9.00 g, 23.7 mmol) in EtOH (150 mL) was added EtONa (6.44 g, 94.7 mmol) at 0° C., the mixture was stirred at 20° C. for 2 hours. The reaction mixture was quenched with 1N aqueous HCl to pH=6 and extracted with EtOAc (200 mL×5). The combined organic layer was dried over anhydrous Na₂SO₄, filtered and concentrated. The residue was purified by flash silica gel chromatography (ISCO®; 40 g SepaFlash® Silica Flash Column, Eluent of 5˜10% MeOH/DCM gradient @60 mL/min) to give 6-bromoisoquinolin-4-ol (4.20 g, yield: 73%) as a light yellow solid.

¹H NMR (400 MHz, DMSO-d₆) δ 7.79 (1H, dd, J=8.8, 2.0 Hz), 8.02 (1H, d, J=8.8 Hz), 8.10 (1H, s), 8.26 (1H, d, J=1.6 Hz), 8.82 (1H, s), 10.64 (1H, brs).

### Intermediate 4

### 5-(isoquinolin-6-yl)thiazol-2-amine

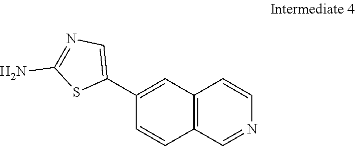 [SMILES: Nc1ncc(-c2ccc3cnccc3c2)s1]

### Step 1. Synthesis of (E)-6-(2-ethoxyvinyl)isoquinoline

A mixture of 6-bromoisoquinoline (1.00 g, 4.81 mmol), 2-[(E)-2-ethoxyvinyl]-4,4,5,5-tetramethyl-1,3,2-dioxaborolane (1.50 g, 7.57 mmol), Pd(dppf)Cl₂ (352 mg, 0.481 mmol), Na₂CO₃ (1.53 g, 14.4 mmol) in dioxane (8 mL) and H₂O (2 mL) was degassed and purged with N₂ for 3 times. Then the mixture was stirred at 90° C. for 16 hours under N₂ atmosphere. The reaction mixture was filtered and the filtrate was concentrated. The residue was purified by flash silica gel chromatography (ISCO®; 20 g SepaFlash® Silica Flash Column, Eluent of 0˜30% Ethyl acetate/Petroleum ethergradient @40 mL/min) to give Int-4c (900 mg, yield: 94%) as a yellow solid.

¹H NMR (400 MHz, DMSO-d₆) δ 1.29 (3H, t, J=6.8 Hz), 3.98 (2H, q, J=6.8 Hz), 6.04 (1H, d, J=12.8 Hz), 7.53 (1H, d, J=12.8 Hz), 7.65 (1H, d, J=6.0 Hz), 7.69 (1H, s), 7.74 (1H, dd, J=8.8, 1.6 Hz), 7.97 (1H, d, J=8.8 Hz), 8.40 (1H, d, J=6.0 Hz), 9.16 (1H, s).

### Step 2. Synthesis of 5-(isoquinolin-6-yl)thiazol-2-amine

To a solution of (E)-6-(2-ethoxyvinyl)isoquinoline (1.10 g, 5.52 mmol) in dioxane (10 mL) and H₂O (10 mL) was added NBS (1.08 g, 6.07 mmol) portion-wise at 0° C. After the addition, the mixture was stirred at 25° C. for 30 minutes, then thiourea (462 mg, 6.07 mmol) was added at 25° C. The resulting mixture was stirred at 100° C. for 1 hour. The reaction mixture was filtered and the filtrate was concentrated. The residue was purified by flash silica gel chromatography (ISCO®; 12 g SepaFlash® Silica Flash Column, Eluent of 0˜15% MeOH/DCM @25 mL/min), then triturated with MeOH (10 mL) to give 5-(isoquinolin-6-yl)thiazol-2-amine (500 mg, yield: 40%) as a yellow solid.

¹H NMR (400 MHz, DMSO-d₆) δ 7.67 (2H, brs), 7.81-7.91 (2H, m), 8.00 (1H, d, J=6.0 Hz), 8.07 (1H, dd, J=8.8, 1.6 Hz), 8.19 (1H, d, J=8.8 Hz), 8.49 (1H, d, J=6.0 Hz), 9.39 (1H, s).

### Intermediate 5

### 6-(tert-butoxycarbonyl)-2-methyl-1-oxo-1,2,5,6,7,8-hexahydro-2,6-naphthyridine-4-carboxylic acid

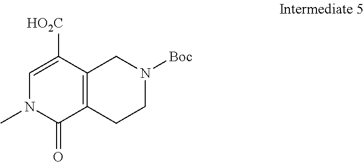 [SMILES: Cn1cc(C(=O)O)c2c(c1=O)CCN(C(=O)OC(C)(C)C)C2]

### Step 1. Synthesis of (E)-3-(2-(dimethylamino)vinyl)isonicotinonitrile

To a solution of 3-methylisonicotinonitrile (4.66 g, 39.4 mmol) in anhydrous DMF (50 mL) was added DMF-DMA (9.40 g, 78.9 mmol) at 20° C. The mixture was stirred at 145° C. for 16 hours. The reaction mixture was concentrated to give (E)-3-(2-(dimethylamino)vinyl)isonicotinonitrile (6.80 g, yield: 99%) as a brown solid.

¹H NMR (400 MHz, DMSO-d₆) δ 2.92 (6H, s), 5.03 (1H, d, J=13.2 Hz), 7.45 (1H, dd, J=5.2, 0.8 Hz), 7.64 (1H, d, J=13.2 Hz), 8.08 (1H, d, J=5.2 Hz), 8.87-8.95 (1H, m).

### Step 2. Synthesis of 2,6-naphthyridin-1(2H)-one

To a solution of (E)-3-(2-(dimethylamino)vinyl)isonicotinonitrile (6.80 g, 39.3 mmol) in EtOH (70 mL) was added HBr (46.3 g, 274 mmol, 48% aqueous) at 20° C. The mixture was stirred at 80° C. for 16 hours. The reaction mixture was concentrated, then diluted with DCM (100 mL) and extracted with DCM (50 mL×3). The combined organic layer was washed with brine (50 mL), dried over anhydrous Na₂SO₄, filtered and concentrated. The residue was purified by Combi Flash (0% to 100% EtOAc in PE) to give 2,6-naphthyridin-1(2H)-one (4.32 g, yield: 75%) as a light brown solid.

¹H NMR (400 MHz, DMSO-d₆) δ 6.67 (1H, d, J=7.2 Hz), 7.32 (1H, d, J=7.2 Hz), 7.96 (1H, d, J=5.6 Hz), 8.61 (1H, d, J=5.2 Hz), 9.06 (1H, d, J=0.8 Hz), 11.63 (1H, brs).

### Step 3. Synthesis of 2-methyl-2,6-naphthyridin-1(2H)-one

To a solution of 2,6-naphthyridin-1(2H)-one (4.32 g, 29.6 mmol) in anhydrous DMF (50 mL) was added NaH (4.73 g, 118 mmol, 60% dispersion in mineral oil) and Mel (9.44 g, 66.5 mmol) at 0° C. The mixture was stirred at 0° C. for 4 hours, then at 20° C. for 18 hours. The reaction mixture was quenched by addition MeOH (30 mL) at 0° C., then concentrated. The residue was purified by Combi Flash (0% to 100% EtOAc in PE) to give 2-methyl-2,6-naphthyridin-1(2H)-one (1.60 g, yield: 33%) as a light yellow solid.

¹H NMR (400 MHz, DMSO-d₆) δ 3.53 (3H, s), 6.73 (1H, d, J=7.2 Hz), 7.62 (1H, d, J=7.2 Hz), 8.00 (1H, d, J=5.2 Hz), 8.63 (1H, d, J=5.2 Hz), 9.06 (1H, d, J=0.8 Hz).

### Step 4. Synthesis of 2-methyl-5,6,7,8-tetrahydro-2,6-naphthyridin-1(2H)-one

A mixture of 2-methyl-2,6-naphthyridin-1(2H)-one (1.60 g, 9.99 mmol) and PtO₂ (1.13 g, 4.99 mmol) in anhydrous EtOH (20 mL) was degassed and purged with H₂ for 3 times, and then the mixture was stirred at 20° C. for 18 hours under H₂ atmosphere (50 psi). The reaction mixture was filtered through a pad of celite and the filtrate was concentrated to give 2-methyl-5,6,7,8-tetrahydro-2,6-naphthyridin-1(2H)-one (1.63 g, yield: 99%) as a light gray solid.

¹H NMR (400 MHz, DMSO-d₆) δ 2.27 (2H, t, J=5.2 Hz), 2.84 (2H, t, J=5.6 Hz), 3.38 (3H, s), 3.57 (2H, s), 5.92 (1H, d, J=7.2 Hz), 7.42 (1H, d, J=6.8 Hz).

### Step 5. Synthesis of tert-butyl 6-methyl-5-oxo-3,4,5,6-tetrahydro-2,6-naphthyridine-2(1H)-carboxylate

To a solution of 2-methyl-5,6,7,8-tetrahydro-2,6-naphthyridin-1(2H)-one (1.63 g, 9.93 mmol) in anhydrous DCM (20 mL) was added TEA (3.01 g, 29.8 mmol) and Boc₂O (2.38 g, 10.9 mmol). The mixture was stirred at 20° C. for 1 hour. The reaction mixture was diluted with water (30 mL) and extracted with DCM (30 mL×3). The combined organic layer was washed with brine (30 mL), dried over anhydrous Na₂SO₄, filtered and concentrated. The residue was purified by Combi Flash (0% to 80% EtOAc in PE) to give tert-butyl 6-methyl-5-oxo-3,4,5,6-tetrahydro-2,6-naphthyridine-2(1H)-carboxylate (2.49 g, yield: 91%) as colorless oil.

¹H NMR (400 MHz, DMSO-d₆) δ 1.41 (9H, s), 2.41 (2H, t, J=5.6 Hz), 3.39 (3H, s), 3.49 (2H, t, J=5.6 Hz), 4.28 (2H, s), 6.06 (1H, d, J=6.8 Hz), 7.51 (1H, d, J=7.2 Hz).

### Step 6. Synthesis of tert-butyl 8-bromo-6-methyl-5-oxo-3,4,5,6-tetrahydro-2,6-naphthyridine-2(1H)-carboxylate

To a solution of tert-butyl 6-methyl-5-oxo-3,4,5,6-tetrahydro-2,6-naphthyridine-2(1H)-carboxylate (2.49 g, 9.42 mmol) in MeCN (30 mL) was added NBS (1.84 g, 10.4 mmol). The mixture was stirred at 20° C. for 1 hour. The reaction mixture was concentrated. The residue was diluted with water (30 mL) and extracted with DCM (30 mL×3). The combined organic layer was washed with brine (30 mL), dried over anhydrous Na₂SO₄, filtered and concentrated. The residue was purified by Combi Flash (0% to 60% EtOAc in PE) to give compound 7 tert-butyl 8-bromo-6-methyl-5-oxo-3,4,5,6-tetrahydro-2,6-naphthyridine-2(1H)-carboxylate (3.09 g, yield: 93%) as a light yellow solid.

¹H NMR (400 MHz, DMSO-d₆) δ 1.42 (9H, s), 2.39-2.48 (2H, t, J=5.6 Hz), 3.36 (3H, s), 3.50 (2H, t, J=5.6 Hz), 4.25 (2H, s), 7.99 (1H, s).

### Step 7. Synthesis of 2-(tert-butyl) 8-ethyl 6-methyl-5-oxo-3,4,5,6-tetrahydro-2,6-naphthyridine-2,8(1H)-dicarboxylate

A mixture of tert-butyl 8-bromo-6-methyl-5-oxo-3,4,5,6-tetrahydro-2,6-naphthyridine-2(1H)-carboxylate (1.00 g, 2.91 mmol), Pd(OAc)₂ (131 mg, 0.582 mmol), K₂CO₃ (805 mg, 5.83 mmol) and dppp (480 mg, 1.17 mmol) in anhydrous DMF (2 mL) and EtOH (10 mL) was degassed and purged with CO for 3 times, and then the mixture was stirred at 80° C. for 18 hours under CO atmosphere (50 psi). The reaction mixture was filtered through a pad of celite, the filtrate was concentrated. The residue was purified by Combi Flash (0% to 50% EtOAc in PE) to give 2-(tert-butyl) 8-ethyl 6-methyl-5-oxo-3,4,5,6-tetrahydro-2,6-naphthyridine-2,8(1H)-dicarboxylate (860 mg, yield: 56%) as light yellow oil.

### Step 8. Synthesis of 6-(tert-butoxycarbonyl)-2-methyl-1-oxo-1,2,5,6,7,8-hexahydro-2,6-naphthyridine-4-carboxylic acid

To a solution of 2-(tert-butyl) 8-ethyl 6-methyl-5-oxo-3,4,5,6-tetrahydro-2,6-naphthyridine-2,8(1H)-dicarboxylate (860 mg, 2.56 mmol) in THF (10 mL) and H₂O (2.5 mL) was added LiOH·H₂O (214 mg, 5.11 mmol) at 20° C. The mixture was stirred at 20° C. for 12 hours. The reaction mixture was diluted with H₂O (25 mL), washed with EtOAc (25 mL×2). The aqueous layer was acidified with 1 N aqueous HCl to pH=5, then extracted with EtOAc (30 mL×2) and DCM/MeOH (30 mL×3, 10/1). The combined organic layer was dried over anhydrous Na₂SO₄, filtered and concentrated to give 6-(tert-butoxycarbonyl)-2-methyl-1-oxo-1,2,5,6,7,8-hexahydro-2,6-naphthyridine-4-carboxylic acid (200 mg, yield: 24%) as a white solid.

### Intermediate 6

### 5-(4-chloroisoquinolin-6-yl)thiazol-2-amine

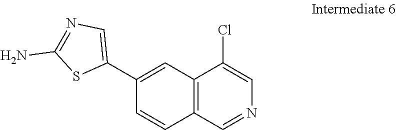 [SMILES: Nc1ncc(-c2ccc3cncc(Cl)c3c2)s1]

### Step 1. Synthesis of 6-bromo-4-chloroisoquinoline

A solution of 6-bromoisoquinoline (500 mg, 2.40 mmol) in SO₂Cl₂ (5 mL) was stirred at 40° C. for 16 hours under N₂ atmosphere. The reaction mixture was added dropwise into saturated aqueous Na₂CO₃ (50 mL) and extracted with EtOAc (20 mL×3). The combined organic layer was dried over anhydrous Na₂SO₄, filtered and concentrated. The residue was purified by flash silica gel chromatography (ISCO®; 12 g SepaFlash® Silica Flash Column, Eluent of 0˜7% Ethyl acetate/Petroleum ethergradient @30 mL/min) to give 6-bromo-4-chloroisoquinoline (270 mg, yield: 46%) as a yellow solid.

¹H NMR (400 MHz, CDCl₃) δ 7.80 (1H, dd, J=8.4, 1.6 Hz), 7.90 (1H, d, J=8.4 Hz), 8.41 (1H, s), 8.63 (1H, s), 9.14 (1H, s).

### Step 2. Synthesis of (E)-4-chloro-6-(2-ethoxyvinyl)isoquinoline

A mixture of 6-bromo-4-chloroisoquinoline (270 mg, 1.11 mmol), 2-[(E)-2-ethoxyvinyl]-4,4,5,5-tetramethyl-1,3,2-dioxaborolane (331 mg, 1.67 mmol), Pd(dppf)Cl₂ (81 mg, 0.11 mmol) and Na₂CO₃ (354 mg, 3.34 mmol) in dioxane (4 mL) and H₂O (1 mL) was degassed and purged with N₂ for 3 times. Then the mixture was stirred at 90° C. for 16 hours under N₂ atmosphere. The reaction mixture was filtered and the filtrate was concentrated. The residue was purified by flash silica gel chromatography (ISCO®; 12 g SepaFlash® Silica Flash Column, Eluent of 0˜15% Ethyl acetate/Petroleum ethergradient @40 mL/min) to give (E)-4-chloro-6-(2-ethoxyvinyl)isoquinoline (140 mg, yield: 54%) as a yellow solid.

### Step 3. Synthesis of 5-(4-chloroisoquinolin-6-yl)thiazol-2-amine

To a solution of (E)-4-chloro-6-(2-ethoxyvinyl)isoquinoline (140 mg, 0.599 mmol) in dioxane (5 mL) and H₂O (5 mL) was added NBS (117 mg, 0.659 mmol) at 0° C. The mixture was stirred at 25° C. for 30 minutes. Then thiourea (50 mg, 0.66 mmol) was added at 25° C. The resulting mixture was stirred at 100° C. for 1 hour. The reaction mixture was filtered and the filtrate was concentrated. The residue was triturated with MeOH (2 mL) to give 5-(4-chloroisoquinolin-6-yl)thiazol-2-amine (100 mg, yield: 64%) as a yellow solid.

### Intermediate 7

### 5-(4-methylisoquinolin-6-yl)thiazol-2-amine

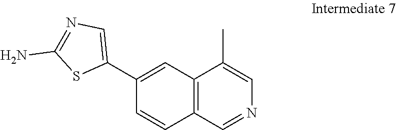 [SMILES: Cc1cncc2ccc(-c3cnc(N)s3)cc12]

### Step 1. Synthesis of 6-bromo-4-methylisoquinoline

A mixture of compound Int-2 (827 mg, 3.29 mmol), trimethylboroxine (1.10 g, 3.29 mmol), Pd(dppf)Cl₂ (241 mg, 0.329 mmol) and K₃PO₄ (1.40 g, 6.59 mmol) in 1,4-dioxane (30 mL) was degassed and purged with N₂ for 3 times, and then the mixture was stirred at 90° C. for 12 hours under N₂ atmosphere. The reaction mixture was concentrated and the residue was diluted with water (25 mL) and extracted with EtOAc (50 mL×2). The combined organic layers were dried over anhydrous Na₂SO₄, filtered and concentrated. The residue was purified by Combi Flash (0% to 15% EtOAc in PE) to give 6-bromo-4-methylisoquinoline (250 mg, yield: 34%) as yellow oil.

### Step 2. Synthesis of (E)-6-(2-ethoxyvinyl)-4-methylisoquinoline

A mixture of 6-bromo-4-methylisoquinoline (250 mg, 1.13 mmol), (E)-1-ethoxyethene-2-boronic acid pinacol ester (267 mg, 1.35 mmol), Na₂CO₃ (239 mg, 2.25 mmol) and Pd(dppf)Cl₂ (83 mg, 0.11 mmol) in 1,4-dioxane (10 mL) and H₂O (1 mL) was degassed and purged with N₂ for 3 times, and then the mixture was stirred at 80° C. for 12 hours under N₂ atmosphere. The reaction mixture was concentrated under reduced pressure to remove solvent. The residue was diluted with water (20 mL) and extracted with EtOAc (20 mL×2). The combined organic layers were dried over anhydrous Na₂SO₄, filtered and concentrated. The reaction mixture was concentrated and the residue was purified by Combi Flash (0% to 30% EtOAc in PE) to give (E)-6-(2-ethoxyvinyl)-4-methylisoquinoline (200 mg, yield: 83%) as yellow oil.

### Step 3. Synthesis of 5-(4-methylisoquinolin-6-yl)thiazol-2-amine

To a solution of (E)-6-(2-ethoxyvinyl)-4-methylisoquinoline (200 mg, 937.76 umol) in 1,4-dioxane (3 mL) and H₂O (3 mL) was added NBS (184 mg, 1.03 mmol) at 0° C. Then the reaction mixture was stirred at 25° C. for 0.5 hour. Thiourea (79 mg, 1.0 mmol) was added and the resulting reaction mixture was stirred at 100° C. for 1.5 hours. The reaction mixture was concentrated and the crude product was triturated with MeOH (10 mL) to give 5-(4-methylisoquinolin-6-yl)thiazol-2-amine (150 mg, yield: 66%) as a yellow solid.

### Intermediate 8

### 6-bromo-4-methoxyisoquinoline

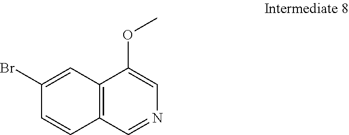

To a solution compound Int-3 (300 mg, 1.34 mmol) in anhydrous DMF (4 mL) was added NaH (64 mg, 1.6 mmol, 60% dispersion on mineral oil) at 0° C., the mixture was stirred at 0° C. for 0.5 hour. Mel (247 mg, 1.74 mmol) was added to the above reaction mixture at 0° C. and stirred at 0° C. for 0.5 hour. The reaction mixture was quenched with H₂O (30 mL) and extracted with EtOAc (30 mL×3). The combined organic layer was washed with brine (40 mL), dried over anhydrous Na₂SO₄, filtered and concentrated. The residue was purified by flash silica gel chromatography (ISCO®; 4 g SepaFlash® Silica Flash Column, Eluent of 20%˜25% Ethyl acetate/Petroleum ether gradient @25 mL/min) to give 6-bromo-4-methoxyisoquinoline (60 mg, yield: 19%) as a white solid.

¹H NMR (400 MHz, DMSO-d₆) δ 4.05 (3H, s), 7.85 (1H, dd, J=8.8, 2.0 Hz), 8.09 (1H, d, J=8.8 Hz), 8.21-8.28 (2H, m), 8.97 (1H, s).

### Intermediate 9

### 5-(4-methoxyisoquinolin-6-yl)thiazol-2-amine

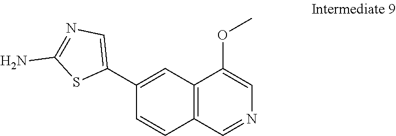 [SMILES: COc1cncc2ccc(-c3cnc(N)s3)cc12]

### Step 1. Synthesis of tert-butyl (5-bromothiazol-2-yl)(4-methoxybenzyl)carbamate

To a mixture of tert-butyl (5-bromothiazol-2-yl)carbamate (2.00 g, 7.16 mmol), PMBOH (1.98 g, 14.3 mmol) and PPh₃ (4.13 g, 15.8 mmol) in THF (20 mL) was added DIAD (3.19 g, 15.8 mmol) at 0° C., the mixture was stirred at 0° C. for 15 minutes, then at 20° C. for 2 hours under N₂ atmosphere. The reaction mixture was concentrated and the residue was purified by flash silica gel chromatography (ISCO®; 20 g SepaFlash® Silica Flash Column, Eluent of 0˜2% Ethyl acetate/Petroleum ether gradient @35 mL/min) to give tert-butyl (5-bromothiazol-2-yl)(4-methoxybenzyl)carbamate (2.20 g, yield: 77%) as a white solid.

### Step 2. Synthesis of tert-butyl (4-methoxybenzyl)(5-(4,4,5,5-tetramethyl-1,3,2-dioxaborolan-2-yl)thiazol-2-yl)carbamate

To a solution of tert-butyl (5-bromothiazol-2-yl)(4-methoxybenzyl)carbamate (1.70 g, 4.26 mmol) in anhydrous THF (20 mL) was added n-BuLi (1.7 mL, 4.25 mmol, 2.5M in hexane) dropwise at −78° C. under N₂ atmosphere, the mixture was stirred at −78° C. for 0.5 hour. Bis-Pin (1.41 g, 5.53 mmol) in anhydrous THF (5 mL) was added to the reaction mixture, the mixture was stirred at −78° C. for another 1.5 hours under N₂ atmosphere. The reaction mixture was quenched with saturated aqueous NH₄Cl (20 mL) and diluted with H₂O (20 mL), extracted with EtOAc (40 mL×3). The combined organic layer was washed with brine (60 mL), dried over anhydrous Na₂SO₄, filtered and concentrated. The residue was purified by flash silica gel chromatography (ISCO®; 40 g SepaFlash® Silica Flash Column, Eluent of 8˜40% Ethyl acetate/Petroleum ether gradient @45 mL/min) to give tert-butyl (4-methoxybenzyl)(5-(4,4,5,5-tetramethyl-1,3,2-dioxaborolan-2-yl)thiazol-2-yl)carbamate (1.30 g, yield: 68%) as a light yellow solid.

### Step 3. Synthesis of tert-butyl (4-methoxybenzyl)(5-(4-methoxyisoquinolin-6-yl)thiazol-2-yl)carbamate

To a solution of tert-butyl (4-methoxybenzyl)(5-(4,4,5,5-tetramethyl-1,3,2-dioxaborolan-2-yl)thiazol-2-yl)carbamate (120 mg, 0.504 mmol) and compound Int-8 (450 mg, 1.01 mmol) in 1,4-dioxane (6 mL) and H₂O (1.2 mL) was added Pd(dppf)Cl₂ (55 mg, 0.076 mmol) and Na₂CO₃ (107 mg, 1.01 mmol) under N₂ atmosphere, the mixture was stirred at 90° C. for 12 hours under N₂ atmosphere. The reaction mixture was concentrated to remove the solvent. The residue was purified by flash silica gel chromatography (ISCO®; 12 g SepaFlash® Silica Flash Column, Eluent of 55˜58% Ethyl acetate/Petroleum ether gradient @25 mL/min) to give tert-butyl (4-methoxybenzyl)(5-(4-methoxyisoquinolin-6-yl)thiazol-2-yl)carbamate (160 mg, yield: 67%) as a light yellow solid.

¹H NMR (400 MHz, CDCl₃) δ 1.56 (9H, s), 3.80 (3H, s), 4.09 (3H, s), 5.30 (2H, s), 6.81-6.88 (2H, m), 7.30-7.39 (2H, m), 7.82-7.88 (2H, m), 7.93 (1H, d, J=8.4 Hz), 8.07 (1H, s), 8.31 (1H, s), 8.85 (1H, s).

### Step 4. Synthesis of 5-(4-methoxyisoquinolin-6-yl)thiazol-2-amine

A solution of tert-butyl (4-methoxybenzyl)(5-(4-methoxyisoquinolin-6-yl)thiazol-2-yl)carbamate (160 mg, 0.335 mmol) in TFA (5 mL) was stirred at 60° C. for 16 hours. The reaction mixture was concentrated and the residue was basified with 2N aqueous NaOH to pH=10 and diluted into H₂O (30 mL), extracted with DCM/MeOH (30 mL×3, 10/1). The combined organic layer was dried over anhydrous Na₂SO₄, filtered and concentrated. The residue was purified by flash silica gel chromatography (ISCO®; 4 g SepaFlash® Silica Flash Column, Eluent of 6˜7% MeOH/DCM gradient @25 mL/min) to give 5-(4-methoxyisoquinolin-6-yl)thiazol-2-amine (54 mg, yield: 63%) as a yellow solid.

### Intermediate 10

### 5-(5-chloroisoquinolin-6-yl)thiazol-2-amine

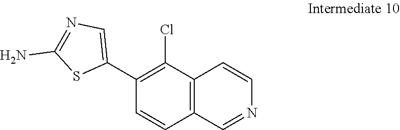 [SMILES: Nc1ncc(-c2ccc3cnccc3c2Cl)s1]

### Step 1. Synthesis of 6-bromo-5-chloroisoquinoline

To a mixture of 6-bromoisoquinoline (500 mg, 2.40 mmol) in conc. H₂SO₄ (10 mL) was added NCS (1.80 g, 13.5 mmol) at 0° C., then the mixture was stirred at 50° C. for 24 hours under N₂ atmosphere. The reaction mixture was poured into saturated aqueous Na₂CO₃ (100 mL) slowly at 0° C. and extracted with EA (50 mL×3). The combined organic layer was dried over anhydrous Na₂SO₄, filtered and concentrated to give 6-bromo-5-chloroisoquinoline (580 mg, yield: >99%) as a yellow solid.

¹H NMR (400 MHz, CDCl₃) δ 7.76-7.80 (1H, m), 7.80-7.85 (1H, m), 8.06 (1H, d, J=6.0 Hz), 8.68 (1H, d, J=6.0 Hz), 9.26 (1H, s).

### Step 2. Synthesis of (E)-5-chloro-6-(2-ethoxyvinyl)isoquinoline

A mixture of 6-bromo-5-chloroisoquinoline (300 mg, 1.24 mmol), 2-[(E)-2-ethoxyvinyl]-4,4,5,5-tetramethyl-1,3,2-dioxaborolane (368 mg, 1.86 mmol), Pd(dppf)Cl₂ (91 mg, 0.12 mmol) and Na₂CO₃ (393 mg, 3.71 mmol) in dioxane (4 mL) and H₂O (1 mL) was degassed and purged with N₂ for 3 times. Then the mixture was stirred at 90° C. for 16 hours under N₂ atmosphere. The reaction mixture was filtered and the filtrate was concentrated. The residue was purified by flash silica gel chromatography (ISCO®; 20 g SepaFlash® Silica Flash Column, Eluent of 0˜16% Ethyl acetate/Petroleum ether gradient @40 mL/min) to give (E)-5-chloro-6-(2-ethoxyvinyl)isoquinoline (240 mg, yield: 83%) as a yellow solid.

### Step 3. Synthesis of 5-(5-chloroisoquinolin-6-yl)thiazol-2-amine

To a solution of (E)-5-chloro-6-(2-ethoxyvinyl)isoquinoline (240 mg, 1.03 mmol) in dioxane (3 mL) and H₂O (3 mL) was added NBS (201 mg, 1.13 mmol) at 0° C. The mixture was stirred at 25° C. for 30 minutes. Then thiourea (86 mg, 1.1 mmol) was added at 25° C. The resulting mixture was stirred at 100° C. for 1 hour. The reaction mixture was filtered and the filtrate was concentrated. The residue was triturated with EtOAc (3 mL) to give 5-(5-chloroisoquinolin-6-yl)thiazol-2-amine (210 mg, yield: 78%) as a yellow solid.

### Intermediate 11

### 6-bromo-5-methylisoquinoline

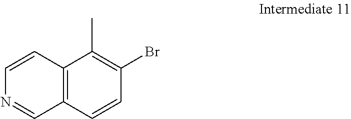

### Step 1. Synthesis of (3-bromo-2-methylphenyl)methanol

To a solution of 3-bromo-2-methylbenzoic acid (10.0 g, 46.5 mmol) in anhydrous THE (100 mL) was added BH₃·Me₂S (6.98 mL, 10 M) at 0° C. The mixture was stirred at 25° C. for 12 hours. The reaction mixture was quenched with 1 N aqueous HCl (30 mL) and extracted with EtOAc (100 mL×3). The combined organic layer was washed with brine (50 mL), dried over anhydrous Na₂SO₄, filtered and concentrated to give (3-bromo-2-methylphenyl)methanol (9.00 g, yield: 96%) as colorless oil.

¹H NMR (400 MHz, CDCl₃) δ 2.45 (3H, s), 4.74 (2H, s), 7.08 (1H, t, J=7.6 Hz), 7.34 (1H, d, J=7.2 Hz), 7.53 (1H, d, J=8.0 Hz).

### Step 2. Synthesis of 1-bromo-3-(bromomethyl)-2-methylbenzene

To a solution of (3-bromo-2-methylphenyl)methanol (9.00 g, 44.8 mmol) in DCM (100 mL) was added PBr₃ (12.1 g, 44.8 mmol) at 0° C. The mixture was stirred at 25° C. for 3 hours.

The reaction mixture was quenched with saturated aqueous Na₂CO₃ (70 mL) at 25° C. and extracted with EtOAc (70 mL×3). The combined organic layer was washed with brine (50 mL), dried over anhydrous Na₂SO₄, filtered and concentrated. The residue was purified by silica gel column (PE/EtOAc=1/0 to 10/1) to give 1-bromo-3-(bromomethyl)-2-methylbenzene (10.0 g, yield: 85%) as colorless oil.

¹H NMR (400 MHz, CDCl₃) δ 2.40 (3H, s), 4.44 (2H, s), 6.94 (1H, t, J=7.6 Hz), 7.17 (1H, d, J=6.4 Hz), 7.44 (1H, d, J=8.0 Hz).

### Step 3. Synthesis of 2-(3-bromo-2-methylphenyl)acetonitrile

To a solution of 1-bromo-3-(bromomethyl)-2-methylbenzene (10.0 g, 37.9 mmol) in MeOH (100 mL) was added KF (11.0 g, 189 mmol) and TMSCN (18.8 g, 189 mmol) at 0° C. The mixture was stirred at 25° C. for 12 hours. The reaction mixture was concentrated and the residue was diluted with water (40 mL) and extracted with EtOAc (70 mL×3). The combined organic layer was washed with brine (50 mL), dried over anhydrous Na₂SO₄, filtered and concentrated. The residue was purified by silica gel column (PE/EtOAc=10/1 to 5/1) to give 2-(3-bromo-2-methylphenyl)acetonitrile (6.50 g, yield: 82%) as a white solid.

¹H NMR (400 MHz, CDCl₃) δ 2.45 (3H, s), 3.74 (2H, s), 7.09 (1H, t, J=7.6 Hz), 7.33 (1H, d, J=7.6 Hz), 7.57 (1H, d, J=8.0 Hz).

### Step 4. Synthesis of 2-(3-bromo-2-methylphenyl)ethan-1-amine

To a solution of 2-(3-bromo-2-methylphenyl)acetonitrile (6.00 g, 28.6 mmol) in anhydrous THF (100 mL) was added BH₃·Me₂S (5.71 mL, 10 M) at 0° C. The mixture was stirred at 25° C. for 12 hours. The reaction mixture was quenched with 1 N aqueous HCl (20 mL) at 25° C. and concentrated. Then the mixture was basified with 2 N aqueous NaOH to pH=10 and extracted with EtOAc (100 mL×3). The combined organic layer was washed with brine (50 mL), dried over anhydrous Na₂SO₄, filtered and concentrated to give 2-(3-bromo-2-methylphenyl)ethan-1-amine (5.00 g, yield: 82%) as colorless oil.

### Step 5. Synthesis of N-(3-bromo-2-methylphenethyl)formamide

To a solution of HCOOH (2.24 g, 46.7 mmol) in THF (100 mL) was added CDI (7.57 g, 46.7 mmol) at 0° C. The mixture was stirred at 25° C. for 0.5 hour. Then 2-(3-bromo-2-methylphenyl)ethan-1-amine (5.00 g, 23.4 mmol) was added to the reaction mixture at 0° C. and stirred at 25° C. for another 0.5 hour. The reaction mixture was concentrated under reduced pressure and the residue was purified by silica gel column (PE/EtOAc=3/1 to 1/1) to give N-(3-bromo-2-methylphenethyl)formamide (3.20 g, yield: 57%) as colorless oil.

¹H NMR (400 MHz, CDCl₃) δ 2.45 (3H, s), 2.94 (2H, t, J=7.2 Hz), 3.54 (2H, t, J=6.8 Hz), 5.64 (1H, brs), 6.95-7.05 (1H, m), 7.08-7.13 (1H, m), 7.48 (1H, d, J=7.2 Hz), 8.18 (1H, s).

### Step 6. Synthesis of 6-bromo-5-methyl-3,4-dihydroisoquinoline

A solution of N-(3-bromo-2-methylphenethyl)formamide (1.00 g, 4.13 mmol) in PPA (5 mL) was stirred at 120° C. for 12 hours. The reaction mixture was quenched with 30% aqueous NH₃·H₂O to pH=9 and extracted with EtOAc (60 mL×3). The combined organic layer was washed with brine (25 mL), dried over anhydrous Na₂SO₄, filtered and concentrated to give 6-bromo-5-methyl-3,4-dihydroisoquinoline (800 mg, yield: 86%) as yellow oil.

### Step 7. Synthesis of 6-bromo-5-methylisoquinoline

To a solution of 6-bromo-5-methyl-3,4-dihydroisoquinoline (800 mg, 3.57 mmol) in toluene (10 mL) was added MnO₂ (931 mg, 10.7 mmol) at 25° C. The mixture was stirred at 100° C. for 12 hours. The reaction mixture was filtered and the filtrate was concentrated. The residue was purified by silica gel column (PE/EtOAc=5/1 to 3/1) to give 6-bromo-5-methylisoquinoline (500 mg, yield: 59%) as a yellow oil.

¹H NMR (400 MHz, CDCl₃) δ 2.70 (3H, s), 7.53-7.76 (3H, m), 8.52 (1H, d, J=6.0 Hz), 9.13 (1H, s).

### Intermediate 12

### 5-(3-methylisoquinolin-6-yl)thiazol-2-amine

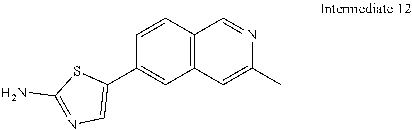 [SMILES: Cc1cc2cc(-c3cnc(N)s3)ccc2cn1]

### Step 1. Synthesis of 6-bromo-3-methylisoquinoline

To a solution of (4-bromophenyl)methanamine (10.0 g, 53.8 mmol) in DCM (100 mL) was added 1, 1-dimethoxypropan-2-one (6.98 g, 59.1 mmol) and MgSO₄ (20.0 g, 166 mmol). The mixture was stirred at 40° C. for 12 hours. The reaction mixture was cooled to 25° C., NaBH₃CN (4.05 g, 64.5 mmol) was added and stirred at 25° C. for 5 hours. The mixture was filtered and the filtrate was concentrated. The residue was cooled to −10° C. and ClSO₃H (52.5 g, 451 mmol) was added dropwise at −10° C. The reaction mixture was heated at 100° C. for 10 minutes and poured into ice. The mixture was basified with 2N aqueous NaOH to pH=10 and extracted with DCM (100 mL×2). The combined organic layers were dried over anhydrous Na₂SO₄, filtered and concentrated. The residue was purified by Combi Flash (0% to 30% EtOAc in PE) to give 6-bromo-3-methylisoquinoline (2.30 g, yield: 19%) as a white solid.

### Step 2. Synthesis of (E)-6-(2-ethoxyvinyl)-3-methylisoquinoline

A mixture of 6-bromo-3-methylisoquinoline (500 mg, 2.25 mmol), 2-[(E)-2-ethoxyvinyl]-4,4,5,5-tetramethyl-1,3,2-dioxaborolane (535 mg, 2.70 mmol) and Pd(dppf)Cl₂ (165 mg, 0.225 mmol), Na₂CO₃ (477 mg, 4.50 mmol) in 1,4-dioxane (10 mL) and H₂O (1 mL) was degassed and purged with N₂ for 3 times, and then the mixture was stirred at 90° C. for 12 hours under N₂ atmosphere. The reaction mixture was diluted with H₂O (25 mL) and extracted with EtOAc (25 mL×2). The combined organic layers were washed with brine (25 mL) and dried over anhydrous Na₂SO₄, filtered and concentrated. The residue was purified by Combi Flash (0% to 30% EtOAc in PE) to give (E)-6-(2-ethoxyvinyl)-3-methylisoquinoline (370 mg, yield: 77%) as a yellow solid.

### Step 3. Synthesis of 5-(3-methylisoquinolin-6-yl)thiazol-2-amine

To a solution of (E)-6-(2-ethoxyvinyl)-3-methylisoquinoline (170 mg, 0.797 mmol) in 1,4-dioxane (2 mL) and H₂O (2 mL) was added NBS (156 mg, 0.877 mmol) at 0° C. After addition, the mixture was stirred at 25° C. for 30 minutes. Thiourea (67 mg, 0.88 mmol) was added and the resulting mixture was stirred at 100° C. for 3.5 hours. The reaction was concentrated and the crude product was triturated with MeOH (10 mL) to give 5-(3-methylisoquinolin-6-yl)thiazol-2-amine (80 mg, yield: 42%) as a yellow solid.

### Intermediate 13

### 6-bromo-1-methylisoquinoline

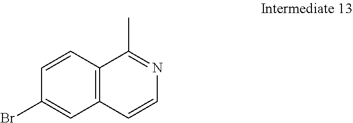

To a mixture of 6-bromo-1-chloroisoquinoline (3.00 g, 12.4 mmol), TMEDA (719 mg, 6.19 mmol), Fe(acac)3 (437 mg, 1.24 mmol) in anhydrous THF (5 mL) was added MeMgBr (5.4 mL, 3 M in Et2O) under N2 at 0° C. Then the mixture was stirred at 25° C. for 16 hours under N₂ atmosphere. The reaction mixture was concentrated and the residue was purified by flash silica gel chromatography (ISCO®; 20 g SepaFlash® Silica Flash Column, Eluent of 0˜7% Ethyl acetate/Petroleum ether gradient @45 mL/min) to give 6-bromo-1-methylisoquinoline (1.90 g, yield: 69%) as a yellow solid.

¹H NMR (400 MHz, CDCl₃) δ 2.87 (3H, s), 7.34 (1H, d, J=6.0 Hz), 7.59 (1H, dd, J=8.8, 2.0 Hz), 7.85-7.95 (2H, m), 8.33 (1H, d, J=6.0 Hz).

### Intermediate 14

### 1-methyl-N-(thiazol-2-yl)piperidine-4-carboxamide

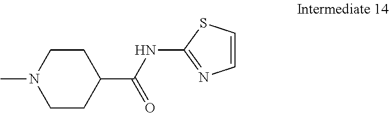

To a suspension of thiazol-2-amine (2.00 g, 20.0 mmol), 1-methylpiperidine-4-carboxylic acid (4.29 g, 30.0 mmol) in pyridine (20 mL) was added Et₃N (4.04 g, 39.9 mmol) and T₃P (38.1 g, 59.9 mmol 50% in EtOAc) at 10-15° C. Then the reaction mixture was stirred at 50° C. for 16 hours. The reaction mixture turned into yellow solution from suspension. The reaction mixture was concentrated and the residue was basified with 1N aqueous NaOH to pH=11, then extracted with EtOAc (100 mL×2). The combined organic layer was washed with brine (100 mL), dried over anhydrous Na₂SO₄ and concentrated. The crude product was triturated with PE/EtOAc (20 mL 1/1) to give 1-methyl-N-(thiazol-2-yl)piperidine-4-carboxamide (3.60 g, yield: 80%) as a yellow solid.

¹H NMR (400 MHz, CDCl₃) δ 1.55-1.70 (2H, m), 1.70-1.80 (2H, m), 1.81-1.91 (2H, m), 2.16 (3H, s), 2.40-2.46 (1H, m), 2.75-2.85 (2H, m), 7.19 (1H, d, J=3.6 Hz), 7.46 (1H, d, J=3.6 Hz), 12.07 (1H, brs).

### Intermediate 15

### 5-(1,6-naphthyridin-2-yl)thiazol-2-amine

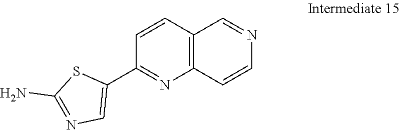 [SMILES: Nc1ncc(-c2ccc3cnccc3n2)s1]

### Step 1. Synthesis of 2-chloro-1,6-naphthyridine

A mixture of 1,6-naphthyridin-2(1H)-one (500 mg, 3.42 mmol) in POCl₃ (8.25 g, 53.8 mmol) was stirred at 120° C. for 3 hours. After cooling to 25° C., the resulting solution was concentrated and the residue was quenched with saturated aqueous NaHCO₃ (100 mL) and extracted with EtOAc (50 mL×2). The combined organic layer was dried over anhydrous Na₂SO₄. The residue was purified by Combi Flash (0% to 30% EtOAc in PE) to give 2-chloro-1,6-naphthyridine (120 mg, yield: 21%) as a light yellow solid.

### Step 2. Synthesis of (E)-2-(2-ethoxyvinyl)-1,6-naphthyridine

A mixture of 2-chloro-1,6-naphthyridine (120 mg, 0.730 mmol), 2-[(E)-2-ethoxyvinyl]-4,4,5,5-tetramethyl-1,3,2-dioxaborolane (173 mg, 0.875 mmol), Pd(dppf)Cl₂ (53 mg, 0.073 mmol) and Na₂CO₃ (155 mg, 1.46 mmol) in 1,4-dioxane (10 mL) and H₂O (1 mL) was degassed and purged with N₂ for 3 times, and then the mixture was stirred at 80° C. for 12 hours under N₂ atmosphere. The reaction mixture was diluted with H₂O (20 mL) and extracted with EtOAc (25 mL×2). The combined organic layers were dried over anhydrous Na₂SO₄, filtered and concentrated. The residue was purified by Combi Flash (0% to 100% EtOAc in PE) to give (E)-2-(2-ethoxyvinyl)-1,6-naphthyridine (70 mg, yield: 48%) as brown oil.

### Step 3. Synthesis of 5-(1,6-naphthyridin-2-yl)thiazol-2-amine

To a solution of (E)-2-(2-ethoxyvinyl)-1,6-naphthyridine (70 mg, 0.35 mmol) in 1, 4-dioxane (3 mL) and H₂O (3 mL) was added NBS (68 mg, 0.39 mmol) at 0° C. After addition, the mixture was stirred at 25° C. for 30 minutes. Thiourea (29 mg, 0.39 mmol) was added and the resulting mixture was stirred at 100° C. for 4.5 hours. The reaction was concentrated and the residue was purified by Combi Flash (0% to 5% MeOH in DCM) to give 5-(1,6-naphthyridin-2-yl)thiazol-2-amine (50 mg, yield: 63%) as a yellow solid.

### Intermediate 16

### 3-chloro-1,7-naphthyridine

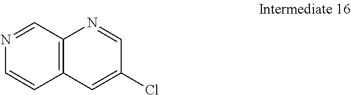 [SMILES: Clc1cnc2cnccc2c1]

### Step 1. Synthesis of 3-bromo-5-chloro-2-methylpyridine

To a solution of 5-bromo-6-methylpyridin-3-amine (2.40 g, 12.8 mmol) in CH₃CN (25 mL) was added isoamyl nitrite (3.01 g, 25.7 mmol) and CuCl₂ (4.31 g, 32.1 mmol) at 25° C. The mixture was stirred at 70° C. for 2 hours. The reaction mixture was quenched with H₂O (50 mL) and extracted with EtOAc (50 mL×3). The combined organic layers were washed with brine (50 mL), dried over anhydrous Na₂SO₄, filtered and concentrated. The residue was purified by column chromatography (SiO₂, Petroleum ether/Ethyl acetate=1/0 to 10/1) to give 3-bromo-5-chloro-2-methylpyridine (2.20 g, yield: 83%) as colorless oil.

¹H NMR (400 MHz, CDCl₃) δ 2.64 (3H, s), 7.83 (1H, d, J=2.0 Hz, 1H), 8.40 (1H, d, J=2.0 Hz).

### Step 2. Synthesis of 3-bromo-2-(bromomethyl)-5-chloropyridine

To a solution of 3-bromo-5-chloro-2-methylpyridine (1.00 g, 4.84 mmol) in DCE (20 mL) was added NBS (1.29 g, 7.27 mmol) and BPO (235 mg, 0.969 mmol) at 25° C. The mixture was stirred at 70° C. for 12 hours. The mixture was concentrated and the residue was purified by column chromatography (SiO₂, Petroleum ether/Ethyl acetate=1/0 to 10/1) to give 3-bromo-2-(bromomethyl)-5-chloropyridine (1.10 g, yield: 72%) as colorless oil.

### Step 3. Synthesis of 3-bromo-5-chloropicolinaldehyde

To a solution of 3-bromo-2-(bromomethyl)-5-chloropyridine (1.10 g, 3.85 mmol) in CH₃CN (12 mL) was added NMO (903 mg, 7.71 mmol) at 25° C. The mixture was stirred at 25° C. for 0.5 hour. The reaction mixture was quenched with H₂O (50 mL) and extracted with EtOAc (50 mL×3). The combined organic layers were washed with brine (50 mL), dried over anhydrous Na₂SO₄, filtered and concentrated. The residue was purified by column chromatography (SiO₂, Petroleum ether/Ethyl acetate=1/0 to 10/1) to give 3-bromo-5-chloropicolinaldehyde (500 mg, yield: 56%) as a light yellow solid.

¹H NMR (400 MHz, CDCl₃) δ 8.00 (1H, d, J=2.0 Hz), 8.64 (1H, d, J=2.0 Hz), 10.12 (1H, s).

### Step 4. Synthesis of 5-chloro-3-((trimethylsilyl)ethynyl)picolinaldehyde

To a solution of 3-bromo-5-chloropicolinaldehyde (400 mg, 1.81 mmol) in THF (5 mL) was added CuI (35 mg, 0.18 mmol), Et₃N (918 mg, 9.07 mmol), Pd(PPh₃)₂Cl₂ (127 mg, 0.181 mmol), ethynyl(trimethyl)silane (267 mg, 2.72 mmol) at 25° C. under N₂ atmosphere. The mixture was stirred at 25° C. for 1 hour under N₂ atmosphere. The reaction mixture was quenched with H₂O (25 mL) and extracted with EtOAc (25 mL×3). The combined organic layers were washed with brine (25 mL), dried over anhydrous Na₂SO₄, filtered and concentrated. The residue was purified by column chromatography (SiO₂, Petroleum ether/Ethyl acetate=1/0 to 10/1) to give 5-chloro-3-((trimethylsilyl)ethynyl)picolinaldehyde (250 mg, yield: 58%) as yellow gum.

¹H NMR (400 MHz, CDCl₃) δ 0.31 (9H, s), 7.92 (1H, d, J=2.0 Hz), 8.66 (1H, d, J=2.0 Hz), 10.36 (1H, s).

### Step 5. Synthesis of 3-chloro-1,7-naphthyridine

A solution of 5-chloro-3-((trimethylsilyl)ethynyl)picolinaldehyde (250 mg, 1.05 mmol) in 7N NH₃/MeOH (20 mL) was stirred at 80° C. for 12 hours in a sealed tube. The reaction mixture was concentrated and the residue was purified by column chromatography (SiO₂, Petroleum ether/Ethyl acetate=10/1 to 5/1) to give 3-chloro-1,7-naphthyridine (50 mg, yield: 29%) as a brown solid.

¹H NMR (400 MHz, CDCl₃) δ 7.56 (1H, d, J=5.6 Hz), 8.10 (1H, d, J=2.0 Hz), 8.60 (1H, d, J=5.6 Hz), 8.88 (1H, d, J=2.4 Hz), 9.46 (1H, s).

### Intermediate 17

### 8-bromo-4H-quinolizin-4-one

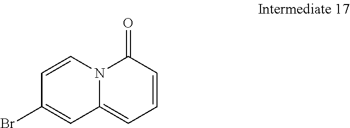 [SMILES: O=c1cccc2cc(Br)ccn12]

### Step 1. Synthesis of ethyl 8-bromo-4-oxo-4H-quinolizine-3-carboxylate

To a solution of LDA (1.9 mL, 3.80 mmol, 2M in THF) in THF (20 mL) was added a solution of 4-bromo-2-methylpyridine (500 mg, 2.91 mmol) in THF (4 mL) dropwise over 10 minutes at −65° C. After stirring at −65° C. for 40 minutes, a solution of diethyl 2-(ethoxymethylene)propanedioate (755 mg, 3.49 mmol) in THF (2 mL) was added dropwise to the reaction mixture over 20 minutes. The mixture was warmed slowly to 25° C. and stirred for another 2.5 hours. The reaction was quenched with saturated aqueous NH₄Cl (25 mL) and extracted with EtOAc (50 mL×2). The combined organic layers were dried over anhydrous Na₂SO₄, filtered and concentrated. The residue was dissolved in toluene (4 mL) and stirred at 60° C. for 12 hours. The reaction mixture was concentrated and the residue was purified by Combi Flash (0% to 60% EtOAc in PE) to give compound 2 (230 mg, yield: 27%) as a yellow solid.

### Step 2. Synthesis of 8-bromo-4H-quinolizin-4-one

A mixture of ethyl 8-bromo-4-oxo-4H-quinolizine-3-carboxylate (330 mg, 1.11 mmol) in 6N aqueous HCl (6 mL) was stirred at 100° C. for 12 hours under N₂ atmosphere. The reaction mixture was neutralized with 1N aqueous NaOH at 0° C. and extracted with EtOAc (25 mL×2).

The combined organic layers were dried over anhydrous Na₂SO₄, filtered and concentrated. The residue was purified by Combi Flash (0% to 60% EtOAc in PE) to give 8-bromo-4H-quinolizin-4-one (70 mg, yield: 28%) as a yellow solid.

### Intermediate 18

### 6-bromo-3-iodopyrazolo[1,5-a]pyridine

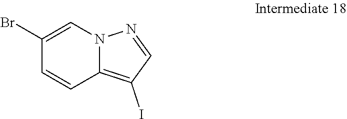 [SMILES: Brc1ccc2c(I)cnn2c1]

To a solution of 6-bromopyrazolo[1,5-a]pyridine (200 mg, 1.02 mmol) in DMF (4 mL) was added NIS (251 mg, 1.12 mmol), then the mixture was stirred at 25° C. for 1 hour. The reaction mixture was poured into water (20 mL) and filtered. The solid was washed with water (10 mL×2) and dried to give 6-bromo-3-iodopyrazolo[1,5-a]pyridine (300 mg, yield: 91%) as a white solid.

¹H NMR (400 MHz, DMSO-d₆) δ 7.40-7.55 (2H, m), 8.15 (1H, s), 9.12-9.21 (1H, m).

### Intermediate 19

### 7-bromo-3-iodoimidazo[1,2-a]pyridine

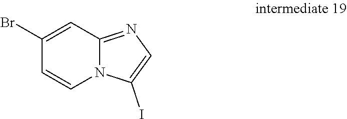 [SMILES: Brc1ccn2c(I)cnc2c1]

To a solution of 7-bromoimidazo[1,2-a]pyridine (900 mg, 4.57 mmol) in DMF (10 mL) was added NIS (1.39 g, 6.17 mmol), the mixture was stirred at 100° C. for 1 hour. The reaction mixture was concentrated and the residue was purified by silica gel column (PE/EtOAc=10/1) to afford compound 2 (850 mg, yield: 58%) as a yellow solid.

¹H NMR (400 MHz, CDCl₃) δ 7.06 (1H, dd, J=7.2, 1.6 Hz), 7.69 (1H, s), 7.84 (1H, d, J=1.2 Hz), 8.01 (1H, d, J=7.6 Hz).

### Intermediate 20

### 3-iodo-6-(trifluoromethyl)pyrazolo[1,5-a]pyridine

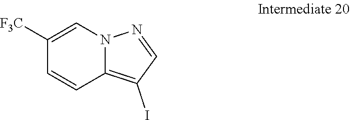 [SMILES: FC(F)(F)c1ccc2c(I)cnn2c1]

### Step 1. Synthesis of 6-iodopyrazolo[1,5-a]pyridine

A mixture of 6-bromopyrazolo[1,5-a]pyridine (2.00 g, 10.2 mmol), CuI (193 mg, 1.02 mmol), NaI (4.56 g, 30.5 mmol) and DMEDA (358 mg, 4.06 mmol) in 1, 4-dioxane (30 mL) was degassed and purged with N₂ for 3 times at 0° C. Then the resulting mixture was stirred at 110° C. for 2.5 days under N₂ atmosphere. The reaction mixture was filtered, the filtrate was cooled to 0° C., then the CuI (193 mg, 1.02 mmol), NaI (4.56 g, 30.5 mmol) and DMEDA (358 mg, 4.06 mmol) was added. The resulting mixture was degassed and purged with N₂ for 3 times at 0° C. and stirred at 110° C. for 4 days under N₂ atmosphere. The reaction mixture was filtered and the filter cake was washed with EtOAc (20 mL×2). The combined organic layers were concentrated and the residue was purified by silica gel column (PE/EtOAc=10/1) to afford 6-iodopyrazolo[1,5-a]pyridine (2.10 g, yield: 68%, purity: 80%) as yellow oil.

¹H NMR (400 MHz, DMSO-d₆) δ 6.58-6.66 (1H, m), 7.36 (1H, dd, J=8.8, 1.2 Hz), 7.54 (1H, d, J=9.2 Hz), 7.93 (1H, d, J=2.0 Hz), 9.03-9.08 (1H, m).

### Step 2. Synthesis of 6-(trifluoromethyl)pyrazolo[1,5-a]pyridine

A mixture of 6-iodopyrazolo[1,5-a]pyridine (2.10 g, 6.88 mmol, purity: 80%), CuI (5.24 g, 27.5 mmol) in DMF (30 mL) was degassed and purged with N₂ for 3 times, then the FSO₂CF₂CO₂Me (5.29 g, 27.5 mmol) was added by syringe. The resulting mixture was stirred at 110° C. for 40 hours under N₂ atmosphere. The reaction mixture was filtered, the filter cake was washed with DMF (15 mL×2), then the CuI (5.24 g, 27.5 mmol) was added to the combined filtrate and the mixture was degassed and purged with N₂ for 3 times, and then the FSO₂CF₂CO₂Me (5.29 g, 27.5 mmol) was added by syringe. The resulting mixture was stirred at 110° C. for 16 hours under N₂ atmosphere. The reaction mixture was filtered and the filtrate was neutralized with saturated aqueous NaHCO₃, adjusted to pH=7, then diluted with H₂O (100 mL) and extracted with EtOAc (100 mL×2). The combined organic layers were dried over anhydrous Na₂SO₄, filtered and concentrated to give 6-(trifluoromethyl)pyrazolo[1,5-a]pyridine (7.20 g, crude) as brown oil.

### Step 3. Synthesis of 3-iodo-6-(trifluoromethyl)pyrazolo[1,5-a]pyridine

A solution of 6-(trifluoromethyl)pyrazolo[1,5-a]pyridine (7.20 g, 7.32 mmol) and NIS (1.81 g, 8.05 mmol) in DMF (30 mL) was stirred at 25° C. for 16 hours. The reaction mixture was diluted with H₂O (100 mL) and extracted with EtOAc (100 mL×2). The combined organic layers were washed with brine (150 mL), dried over anhydrous Na₂SO₄, filtered and concentrated. The residue was purified by silica gel column (PE/EtOAc=10/1) to afford 3-iodo-6-(trifluoromethyl)pyrazolo[1,5-a]pyridine (100 mg, yield: 4%) as a light yellow solid.

¹H NMR (400 MHz, CDCl₃) δ 7.34 (1H, dd, J=9.2, 1.2 Hz), 7.60 (1H, d, J=9.2 Hz), 8.10 (1H, s), 8.81 (1H, s).

### Intermediate 21

### ethyl 4-((6-bromoisoquinolin-4-yl)oxy)cyclohexane-1-carboxylate

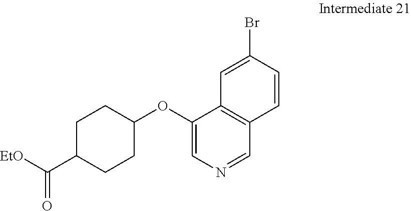 [SMILES: CCOC(=O)C1CCC(Oc2cncc3ccc(Br)cc23)CC1]

A mixture of compound Int-3 (500 mg, 2.23 mmol), ethyl 4-hydroxycyclohexane-1-carboxylate (769 mg, 4.46 mmol), TMAD (1.15 g, 6.69 mmol), n-Bu₃P (1.35 g, 6.69 mmol) in anhydrous toluene (40 mL) was stirred at 110° C. for 16 hours under N₂ atmosphere. The reaction mixture was concentrated under reduced pressure to give a residue. The residue was purified by Combi Flash (0% to 100% EtOAc in PE) to give ethyl 4-((6-bromoisoquinolin-4-yl)oxy)cyclohexane-1-carboxylate (770 mg, yield: 91%) as colorless oil.

### Intermediate 22

### 2-chloro-8-cyclobutoxy-1,6-naphthyridine

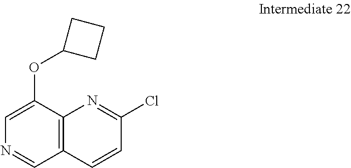 [SMILES: Clc1ccc2cncc(OC3CCC3)c2n1]

### Step 1. Synthesis of 4-bromo-3-cyclobutoxypyridine

To a solution of 4-bromopyridin-3-ol (500 mg, 2.87 mmol) in DMF (5 mL) was added K₂CO₃ (794 mg, 5.75 mmol) and bromocyclobutane (776 mg, 5.75 mmol). The mixture was stirred at 80° C. for 2 hours. The reaction mixture was concentrated and the residue was purified by silica gel column (0% to 18% EtOAc in PE) to give 4-bromo-3-cyclobutoxypyridine (450 mg, yield: 69%) as yellow oil.

¹H NMR (400 MHz, CDCl₃) δ 1.68-1.81 (1H, m), 1.89-1.99 (1H, m), 2.24-2.36 (2H, m), 2.49-2.59 (1H, m), 2.53-2.53 (1H, m), 4.74-4.87 (1H, m), 7.53 (1H, d, J=4.8 Hz), 8.05 (1H, d, J=5.2 Hz), 8.10 (1H, s).

### Step 2. Synthesis of tert-butyl (3-cyclobutoxypyridin-4-yl)carbamate

A mixture of 4-bromo-3-cyclobutoxypyridine (600 mg, 2.63 mmol), BocNH₂ (339 mg, 2.89 mmol), Pd₂(dba)₃ (241 mg, 0.263 mmol), Xantphos (304 mg, 0.526 mmol) and Cs₂CO₃ (2.57 g, 7.89 mmol) in dioxane (10 mL) was degassed and purged with N₂ for 3 times, then the mixture was stirred at 100° C. for 3 hours under N₂ atmosphere. The reaction mixture was suspended in CH₃OH (50 mL) and filtered. The filtrate was concentrated and the residue was purified by silica gel column (0% to 15% EtOAc in PE) to give tert-butyl (3-cyclobutoxypyridin-4-yl)carbamate (600 mg, yield: 74%) as a yellow solid.

### Step 3. Synthesis of 3-cyclobutoxypyridin-4-amine

To a solution of tert-butyl (3-cyclobutoxypyridin-4-yl)carbamate (500 mg, 1.89 mmol) in DCM (10 mL) was added TFA (2 mL). The mixture was stirred at 20° C. for 16 hours. The reaction mixture was concentrated and the residue was diluted with DCM (100 mL) and washed with saturated aqueous NaHCO₃ (50 mL), brine (50 mL), dried over anhydrous Na₂SO₄, filtered and concentrated to give 3-cyclobutoxypyridin-4-amine (350 mg, yield: 94%) as yellow oil.

¹H NMR (400 MHz, DMSO-d₆) δ 1.55-1.68 (1H, m), 1.72-1.82 (1H, m), 1.99-2.15 (2H, m), 2.35-2.45 (2H, m), 4.61-4.73 (1H, m), 5.61 (2H, brs), 6.53 (1H, d, J=4.8 Hz), 7.66-7.74 (2H, m).

### Step 4. Synthesis of 3-bromo-5-cyclobutoxypyridin-4-amine

To a solution of 3-cyclobutoxypyridin-4-amine (300 mg, 1.83 mmol) in CH₃CN (5 mL) was added NBS (358 mg, 2.01 mmol). The mixture was stirred at 20° C. for 1 hour. The reaction mixture was concentrated and the residue was diluted with DCM (100 mL) and washed with saturated aqueous Na₂SO₃ (30 mL×2), saturated aqueous NaHCO₃ (30 mL×2), water (30 mL×2), brine (30 mL), dried over anhydrous Na₂SO₄, filtered and concentrated to give 3-bromo-5-cyclobutoxypyridin-4-amine (350 mg, yield: 79%) as yellow oil.

¹H NMR (400 MHz, DMSO-d₆) δ 1.57-1.68 (1H, m), 1.74-1.84 (1H, m), 2.04-2.16 (2H, m), 2.37-2.47 (2H, m), 4.69-4.81 (1H, m), 5.97 (2H, brs), 7.72 (1H, s), 7.99 (1H, s).

### Step 5. Synthesis of ethyl (E)-3-(4-amino-5-cyclobutoxypyridin-3-yl)acrylate

A mixture of 3-bromo-5-cyclobutoxypyridin-4-amine (350 mg, 1.44 mmol), ethyl (E)-3-(4,4,5,5-tetramethyl-1,3,2-dioxaborolan-2-yl)prop-2-enoate (391 mg, 1.73 mmol), XPhos-Pd-G₃ (122 mg, 0.144 mmol) and K₂CO₃ (398 mg, 2.88 mmol) in dioxane (10 mL) and H₂O (1 mL) was degassed and purged with N₂ for 3 times, and then the mixture was stirred at 90° C. for 16 hours under N₂ atmosphere. The reaction mixture was concentrated and the residue was purified by silica gel column (0% to 50% EtOAc in PE) to give ethyl (E)-3-(4-amino-5-cyclobutoxypyridin-3-yl)acrylate (300 mg, yield: 72%) as a yellow solid.

¹H NMR (400 MHz, DMSO-d₆) δ 1.27 (3H, t, J=7.2 Hz), 1.56-1.69 (1H, m), 1.73-1.85 (1H, m), 2.03-2.16 (2H, m), 2.37-2.45 (2H, m), 4.19 (2H, q, J=7.2 Hz), 4.67-4.80 (1H, m), 6.17 (2H, brs), 6.47 (1H, d, J=16.0 Hz), 7.68 (1H, s), 7.88 (1H, d, J=16.0 Hz), 8.20 (1H, s).

### Step 6. Synthesis of 8-cyclobutoxy-1,6-naphthyridin-2(1H)-one

To a solution of ethyl (E)-3-(4-amino-5-cyclobutoxypyridin-3-yl)acrylate (200 mg, 0.762 mmol) in HOAc (4 mL) was added n-Bu₃P (154 mg, 0.762 mmol). The mixture was stirred at 110° C. for 1 hour. The reaction mixture was concentrated and the residue was triturated with EtOAc (5 mL) to give 8-cyclobutoxy-1,6-naphthyridin-2(1H)-one (200 mg, yield: 81%) as a white solid.

¹H NMR (400 MHz, DMSO-d₆) δ 1.58-1.72 (1H, m), 1.77-1.89 (1H, m), 2.17-2.29 (2H, m), 2.41-2.49 (2H, m), 4.85-4.99 (1H, m), 6.60 (1H, d, J=9.2 Hz), 7.99 (1H, d, J=9.6 Hz), 8.08 (1H, s), 8.48 (1H, s), 11.52 (1H, brs).

### Step 7. Synthesis of 2-chloro-8-cyclobutoxy-1,6-naphthyridine

A mixture of 8-cyclobutoxy-1,6-naphthyridin-2(1H)-one (130 mg, 0.601 mmol) in POCl₃ (3 mL) was stirred at 80° C. for 4 hours. The reaction mixture was concentrated and the residue was diluted with DCM (80 mL) and washed with saturated aqueous NaHCO₃ (30 mL×2), dried over anhydrous Na₂SO₄, filtered and concentrated to give 2-chloro-8-cyclobutoxy-1,6-naphthyridine (150 mg, yield: 92%) as a yellow solid.

¹H NMR (400 MHz, DMSO-d₆) δ 1.64-1.79 (1H, m), 1.83-1.93 (1H, m), 2.17-2.28 (2H, m), 2.55-2.67 (2H, m), 5.01-5.10 (1H, m), 7.92 (1H, d, J=8.4 Hz), 8.37 (1H, s), 8.75 (1H, d, J=8.4 Hz), 9.26 (1H, s).

### Intermediate 23

### 6-(2-methyloxazol-5-yl)isoquinolin-5-amine

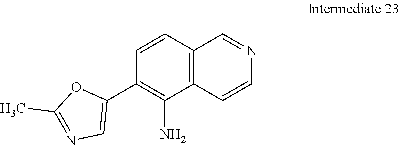 [SMILES: Cc1ncc(-c2ccc3cnccc3c2N)o1]

### Step 1. Synthesis of 6-bromo-5-nitroisoquinoline

To conc. H₂SO₄ (13 mL) was added 6-bromoisoquinoline (2.00 g, 9.61 mmol) slowly at 0° C. After stirring for 6 minutes, KNO₃ (1.02 g, 10.1 mmol) was added in portions and the mixture was stirred at 0° C. for 2 hours. The reaction mixture was added to ice water (150 mL) dropwise. Then 28% aqueous ammonia hydrate was added slowly to adjust pH=9. The precipitated was collected by filtration and dried to give 6-bromo-5-nitroisoquinoline (2.40 g, yield: 88%) as a yellow solid.

### Step 2. Synthesis of 1-(5-nitroisoquinolin-6-yl)ethan-1-one

To a solution of 6-bromo-5-nitroisoquinoline (3.00 g, 11.9 mmol) and tributyl(1-ethoxyvinyl)stannane (6.42 g, 17.8 mmol) in anhydrous toluene (40 mL) was added Pd(PPh₃)₂Cl₂ (832 mg, 1.19 mmol) under N₂ atmosphere, the mixture was stirred at 100° C. for 16 hours under N₂ atmosphere. THF (30 mL) and 3N aqueous HCl (30 mL) were added to the reaction mixture and stirred at 50° C. for 5 hours. The reaction mixture was concentrated and the residue was quenched with saturated aqueous KF (40 mL), basified with 2N aqueous NaOH to pH=9, then extracted with EtOAc (80 mL×2). The combined organic layer was washed with brine (100 mL) and concentrated. The residue was purified by flash silica gel chromatography (ISCO®; 40 g SepaFlash® Silica Flash Column, Eluent of ˜55% Ethyl acetate/Petroleum ether gradient @50 mL/min) to give 1-(5-nitroisoquinolin-6-yl)ethan-1-one (1.90 g, yield: 63%) as a yellow solid.

¹H NMR (400 MHz, CDCl₃) δ 2.70 (3H, s), 7.82 (1H, d, J=8.4 Hz), 7.89 (1H, d, J=6.0 Hz), 8.29 (1H, d, J=8.4 Hz), 8.79 (1H, d, J=6.0 Hz), 9.43 (1H, s).

### Step 3. Synthesis of 2-methyl-5-(5-nitroisoquinolin-6-yl)oxazole

To a solution of TfOH (5.61 g, 37.4 mmol) and 1-(5-nitroisoquinolin-6-yl)ethan-1-one (1.90 g, 7.47 mmol) was added iodosylbenzene (4.11 g, 18.7 mmol) in MeCN (20 mL) at 0° C. and the reaction mixture was stirred at 0° C. for 6 minutes and at 25° C. for 1 hour. The reaction mixture was stirred at 85° C. for 24 hours. Cooled to room temperature, the reaction mixture was concentrated and the residue was diluted with H₂O (20 mL), neutralized with saturated aqueous NaHCO₃ at 0° C., extracted with EtOAc (70 mL×2). The combined organic layer was concentrated and the residue was purified by flash silica gel chromatography (ISCO®; 20 g SepaFlash® Silica Flash Column, Eluent of ˜50% EtOAc/PE gradient @45 mL/min) to give 2-methyl-5-(5-nitroisoquinolin-6-yl)oxazole (2.00 g, yield: 63%) as a yellow solid.

### Step 4. Synthesis of 6-(2-methyloxazol-5-yl)isoquinolin-5-amine

A mixture of 2-methyl-5-(5-nitroisoquinolin-6-yl)oxazole (2.00 g, 4.72 mmol), NH₄Cl (1.01 g, 18.9 mmol) and Fe powder (1.05 g, 18.9 mmol) in EtOH (30 mL) and H₂O (30 mL) was stirred at 75° C. for 2 hours. The reaction mixture was filtered through a pad of celite and the solid was washed with DCM/MeOH (20 mL×3, 10/1). The filtrate was concentrated and the residue was purified by flash silica gel chromatography (ISCO®; 40 g SepaFlash® Silica Flash Column, Eluent of 2-3% DCM/MeOH (1% NH₃·H₂O as an additive) gradient @40 mL/min) to give 6-(2-methyloxazol-5-yl)isoquinolin-5-amine (400 mg, yield: 37%) as a yellow solid.

### Intermediate 24

### ethyl 3-((6-bromoisoquinolin-5-yl)amino)cyclobutane-1-carboxylate

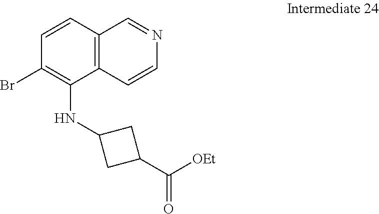 [SMILES: CCOC(=O)C1CC(Nc2c(Br)ccc3cnccc23)C1]

### Step 1. Synthesis of 6-bromoisoquinolin-5-amine

A mixture of 6-bromo-5-nitroisoquinoline (3.00 g, 11.9 mmol) and NH₄Cl (2.54 g, 47.4 mmol) in EtOH (30 mL) and H₂O (30 mL) was added Fe powder (2.65 g, 47.4 mmol) with stirring and then stirred at 75° C. for 1 hour. The reaction mixture was filtered and the filtrate was concentrated. The residue was purified by silica gel column (PE/EtOAc=1/1) to afford 6-bromoisoquinolin-5-amine (2.15 g, yield: 81%) as a pink solid.

¹H NMR (400 MHz, DMSO-d₆) δ 6.15 (2H, brs), 7.23 (1H, d, J=8.8 Hz), 7.62 (1H, d, J=8.4 Hz), 8.10 (1H, d, J=6.0 Hz), 8.45 (1H, d, J=6.0 Hz), 9.15 (1H, s).

### Step 2. Synthesis of ethyl 3-((6-bromoisoquinolin-5-yl)amino)cyclobutane-1-carboxylate

To a solution of 6-bromoisoquinolin-5-amine (500 mg, 2.24 mmol) and ethyl 3-oxocyclobutane-1-carboxylate (574 mg, 4.03 mmol) in DCM (20 mL) was added TiCl₄ (3.40 g, 17.9 mmol) at 0° C. and the reaction mixture was stirred at 0° C. for 5 hours. NaBH₃CN (423 mg, 6.72 mmol) was added to the reaction mixture at 0° C. and the resulting reaction mixture was stirred at 10° C. for 16 hours. The reaction mixture was quenched with MeOH (80 mL), then basified with saturated aqueous NaHCO₃ to pH=8 and filtered. The filtrate was concentrated and the residue was diluted with H₂O (50 mL), then extracted with DCM (50 mL×3). The combined organic layers were dried over anhydrous Na₂SO₄, filtered and concentrated. The residue was purified by silica gel column (PE/EtOAc=2/1) to afford ethyl 3-((6-bromoisoquinolin-5-yl)amino)cyclobutane-1-carboxylate (270 mg, yield: 29%) as a yellow gum.

¹H NMR (400 MHz, DMSO-d₆) δ 1.15-1.18 (3H, m), 2.26-2.37 (2H, m), 2.37-2.48 (2H, m), 2.58-2.81 (1H, m), 3.86-3.98 (1H, m), 4.01-4.10 (3H, m), 7.59 (1H, d, J=8.8 Hz), 7.75 (1H, d, J=8.4 Hz), 7.89-8.00 (1H, m), 8.47-8.58 (1H, m), 9.24 (1H, s).

### Intermediate 25

### methyl (1s,3s)-3-((6-bromoisoquinolin-5-yl)oxy)cyclobutane-1-carboxylate

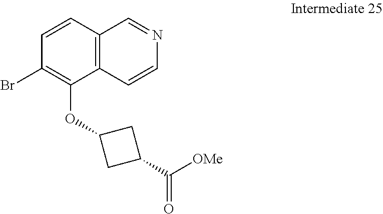 [SMILES: COC(=O)[C@H]1C[C@@H](Oc2c(Br)ccc3cnccc23)C1]

### Step 1. Synthesis of 6-bromoisoquinolin-5-ol

To a solution of isoquinolin-5-ol (1.00 g, 6.89 mmol) in CHCl₃ (18 mL) and MeOH (2 mL) was added the solution of 2,4,4,6-tetrabromocyclohexa-2,5-dien-1-one (2.82 g, 6.89 mmol) in CHCl₃ (54 mL) and MeOH (6 mL) dropwise with stirring over 2 hours at 0° C. and then stirred 25° C. for 16 hours. The reaction mixture was concentrated and the residue was purified by silica gel column (PE/EtOAc=0/1) to afford 6-bromoisoquinolin-5-ol (1.00 g, yield: 39%) as a yellow solid.

¹H NMR (400 MHz, DMSO-d₆) δ 7.58 (1H, d, J=8.8 Hz), 7.75 (1H, d, J=8.8 Hz), 8.04 (1H, d, J=6.0 Hz), 8.53 (1H, d, J=6.0 Hz), 9.26 (1H, s), 10.49 (1H, brs).

### Step 2. Synthesis of methyl (1s,3s)-3-((6-bromoisoquinolin-5-yl)oxy)cyclobutane-1-carboxylate

A mixture of 6-bromoisoquinolin-5-ol (900 mg, 4.02 mmol) and methyl (1r,3r)-3-hydroxycyclobutane-1-carboxylate (627 mg, 4.82 mmol) in toluene (15 mL) was added n-Bu₃P (1.63 g, 8.03 mmol) and TMAD (1.38 g, 8.03 mmol), then stirred at 110° C. for 16 hours under N₂ atmosphere. The reaction mixture was concentrated and the residue was purified by silica gel column (PE/EtOAc=2/1) to afford methyl (1s,3s)-3-((6-bromoisoquinolin-5-yl)oxy)cyclobutane-1-carboxylate (600 mg, yield: 30%) as a yellow oil.

¹H NMR (400 MHz, DMSO-d₆) δ 2.53-2.60 (2H, m), 2.61-2.72 (3H, m), 3.63 (3H, s), 4.47-4.72 (1H, m), 7.85-7.89 (3H, m), 8.60 (1H, d, J=5.6 Hz), 9.36 (1H, s).

### Intermediate 26

### tert-butyl 3-((6-bromo-3-methylisoquinolin-4-yl)oxy)azetidine-1-carboxylate

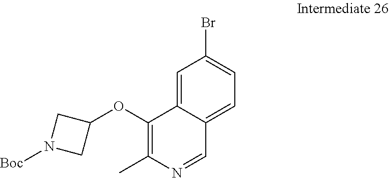 [SMILES: Cc1ncc2ccc(Br)cc2c1OC1CN(C(=O)OC(C)(C)C)C1]

### Step 1. Synthesis of methyl tosylalaninate

To a mixture of methyl alaninate (5.00 g, 35.8 mmol, HCl salt) and Et₃N (10.9 g, 108 mmol) in DCM (150 mL) was added TsCl (6.83 g, 35.8 mmol) portion-wise at 20° C. After the addition, the mixture was stirred at 20° C. for 14 hours under N₂ atmosphere. The reaction mixture was acidified with 1 N aqueous HCl to pH=2 and extracted with DCM (40 mL×3). The combined organic layer was dried over anhydrous Na₂SO₄, filtered and concentrated. The residue was purified by flash silica gel column (PE/EtOAc=3/1) to afford methyl tosylalaninate (7.77 g, yield: 84%) as a colorless oil.

### Step 2. Synthesis of methyl N-(4-bromobenzyl)-N-tosylalaninate

A mixture of methyl tosylalaninate (1.06 g, 4.11 mmol), 4-bromobenzyl bromide (1.03 g, 4.11 mmol) and K₂CO₃ (1.14 g, 8.22 mmol,) in CH₃CN (20 mL) was stirred at 30° C. for 14 hours under N₂ atmosphere. The reaction mixture was filtered and the filtrate was concentrated. The residue was purified by flash silica gel column (PE/EtOAc=5/1) to give methyl N-(4-bromobenzyl)-N-tosylalaninate (1.70 g, yield: 97%) as a white solid.

¹H NMR (400 MHz, CDCl₃) δ 1.27 (3H, d, J=6.4 Hz), 2.44 (3H, s), 3.45 (3H, s), 4.37 (1H, d, J=16.4 Hz), 4.53 (1H, d, J=16.4 Hz), 4.67 (1H, q, J=7.2 Hz), 7.23 (2H, d, J=8.4 Hz), 7.30 (2H, d, J=8.0 Hz), 7.42 (2H, d, J=8.4 Hz), 7.69 (2H, d, J=8.4 Hz).

### Step 3. Synthesis of N-(4-bromobenzyl)-N-tosylalanine

To a solution of methyl N-(4-bromobenzyl)-N-tosylalaninate (1.70 g, 3.99 mmo) in MeOH (10 mL), H₂O (5 mL) and THF (10 mL) was added LiOH·H₂O (502 mg, 12.0 mmol). The mixture was stirred at 20° C. for 1 hour and acidified with 1N aqueous HCl to pH=2˜3, then extracted with DCM (50 mL×3). The combined organic layer was dried over anhydrous Na₂SO₄, filtered and concentrated to give N-(4-bromobenzyl)-N-tosylalanine (1.38 g, yield: 84%) as a white solid.

### Step 4. Synthesis of 6-bromo-3-methyl-2-tosyl-2,3-dihydroisoquinolin-4(H)-one

To a solution of N-(4-bromobenzyl)-N-tosylalanine (2.00 g, 4.85 mmol) in SOCl₂ (24.6 g, 207 mmol) was stirred at 80° C. for 2 hours. The reaction mixture was concentrated and residue was dissolved in anhydrous toluene (15 mL), then concentrated to remove remaining SOCl₂. The residue was dissolved in anhydrous DCM (20 mL) and AlCl₃ (2.48 g, 18.6 mmol) was added portion-wise at 0° C. The resulting reaction mixture was stirred at 10° C. for 16 hours under N₂ atmosphere. The reaction mixture was poured into ice water (50 mL) and then extracted with DCM (50 mL×3). The combined organic layer was dried over anhydrous Na₂SO₄, filtered and concentrated. The residue was purified by flash silica gel column (PE/EtOAc=3/1) to give 6-bromo-3-methyl-2-tosyl-2,3-dihydroisoquinolin-4(1H)-one (160 mg, yield: 9%) as a yellow solid.

### Step 5. Synthesis of 6-bromo-3-methylisoquinolin-4-ol

To a solution of 6-bromo-3-methyl-2-tosyl-2,3-dihydroisoquinolin-4(1H)-one (160 mg, 0.406 mmol) in EtOH (4 mL) was added EtONa (110 mg, 1.62 mmol) at 0° C., then the mixture was stirred at 20° C. for 2 hours. The reaction mixture was quenched with 1N aqueous HCl to pH=7 and diluted with water (10 mL), then extracted with EtOAc (20 mL×3). The combined organic layer was dried over anhydrous Na₂SO₄, filtered and concentrated to give the residue. The residue was purified by flash silica gel column (PE/EtOAc=1/1) to afford 6-bromo-3-methylisoquinolin-4-ol (60 mg, yield: 62%) as a yellow solid.

¹H NMR (400 MHz, DMSO-d₆) δ 2.54 (3H, s), 7.68 (1H, dd, J=8.8, 1.6 Hz), 7.98 (1H, d, J=8.8 Hz), 8.37 (1H, d, J=1.2 Hz), 8.78 (1H, s), 9.58 (1H, brs).

### Step 6. Synthesis of tert-butyl 3-((6-bromo-3-methylisoquinolin-4-yl)oxy)azetidine-1-carboxylate

A mixture of 6-bromo-3-methylisoquinolin-4-ol (400 mg, 1.68 mmol), tert-butyl 3-hydroxyazetidine-1-carboxylate (873 mg, 5.04 mmol), TMAD (868 mg, 5.04 mmol) and n-Bu₃P (1.02 g, 5.04 mmol) in toluene (15 mL) was degassed and purged with N₂ for 3 times, and then the mixture was stirred at 110° C. for 12 hours under N₂ atmosphere. The reaction mixture was concentrated and the residue was diluted with water (20 mL), then extracted with EtOAc (30 mL×3). The combined organic layer was dried over anhydrous Na₂SO₄, filtered and concentrated. The residue was purified by flash silica gel column (PE/EtOAc=1/1) to give tert-butyl 3-((6-bromo-3-methylisoquinolin-4-yl)oxy)azetidine-1-carboxylate (1.04 g, crude) as yellow oil.

### Intermediate 27

### tert-butyl 4-((6-(4,4,5,5-tetramethyl-1,3,2-dioxaborolan-2-yl)isoquinolin-4-yl)oxy)piperidine-1-carboxylate

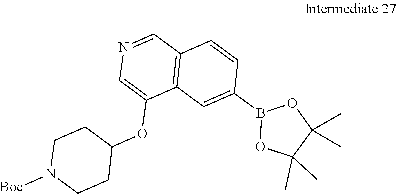 [SMILES: CC(C)(C)OC(=O)N1CCC(Oc2cncc3ccc(B4OC(C)(C)C(C)(C)O4)cc23)CC1]

### Step 1. Synthesis of tert-butyl 4-((6-bromoisoquinolin-4-yl)oxy)piperidine-1-carboxylate

A mixture of compound Int-3 (1.00 g, 4.46 mmol), tert-butyl 4-hydroxypiperidine-1-carboxylate (1.80 g, 8.93 mmol), TMAD (2.31 g, 13.4 mmol) and tributylphosphane (2.71 g, 13.4 mmol) in toluene (40 mL) was degassed and purged with N₂ for 3 times, and then the mixture was stirred at 100° C. for 14 hours under N₂ atmosphere. The reaction mixture was concentrated and diluted with water (40 mL), then extracted with EtOAc (60 mL×3). The combined organic layer was dried over anhydrous Na₂SO₄, filtered and concentrated. The residue was purified by flash silica gel column (PE/EtOAc=1/2) to afford tert-butyl 4-((6-bromoisoquinolin-4-yl)oxy)piperidine-1-carboxylate (2.42 g, crude) as yellow oil.

¹H NMR (400 MHz, CDCl₃) δ 1.47 (9H, s), 1.90-2.00 (2H, m), 2.07-2.14 (2H, m), 2.95-3.10 (2H, m), 3.39-3.49 (2H, m), 4.75-4.85 (1H, m), 7.78-7.86 (1H, m), 7.88-7.95 (1H, m), 8.12 (1H, s), 8.43 (1H, s), 8.91 (1H, s).

### Step 2. Synthesis of tert-butyl 4-((6-(4,4,5,5-tetramethyl-1,3,2-dioxaborolan-2-yl)isoquinolin-4-yl)oxy)piperidine-1-carboxylate

A mixture of tert-butyl 4-((6-bromoisoquinolin-4-yl)oxy)piperidine-1-carboxylate (60 mg, 0.15 mmol), Bis-Pin (75 mg, 0.29 mmol), Pd(dppf)Cl₂ (11 mg, 0.015 mmol) and KOAc (36 mg, 0.37 mmol) in dioxane (4 mL) was degassed and purged with N₂ for 3 times, then the mixture was stirred at 110° C. for 16 hours under N₂ atmosphere. The reaction mixture was filtered and the filtrate was concentrated to give tert-butyl 4-((6-(4,4,5,5-tetramethyl-1,3,2-dioxaborolan-2-yl)isoquinolin-4-yl)oxy)piperidine-1-carboxylate (66 mg, crude) as a black gum.

### Intermediate 28

### 6-bromoisoquinoline-4-carbaldehyde

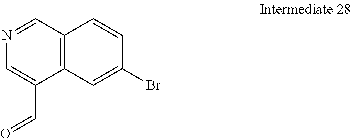 [SMILES: O=Cc1cncc2ccc(Br)cc12]

### Step 1. Synthesis of (6-bromoisoquinolin-4-yl)methanol

A mixture of compound Int-2 (2.50 g, 7.49 mmol), (tributylstannyl)methanol (3.61 g, 11.2 mmol), LiCl (952 mg, 22.4 mmol) and Pd(PPh₃)₂Cl₂ (525 mg, 0.749 mmol) in anhydrous dioxane (20 mL) was degassed and purged with N₂ for 3 times, then the mixture was stirred at 100° C. for 12 hours under N₂ atmosphere. The reaction mixture was concentrated and diluted with saturated aqueous KF (50 mL) and DCM (50 mL), then extracted with DCM (50 mL×3). The combined organic layer was washed with water (50 mL) and brine (50 mL), dried over anhydrous Na₂SO₄, filtered and concentrated. The residue was purified by Combi Flash (0% to 100% EtOAc in PE) to give (6-bromoisoquinolin-4-yl)methanol (1.23 g, yield: 70%) as a light yellow solid.

### Step 2. Synthesis of 6-bromoisoquinoline-4-carbaldehyde

To a solution of (6-bromoisoquinolin-4-yl)methanol (1.23 g, 5.17 mmol) in anhydrous DCM (10 mL) was added Dess-Martin (4.38 g, 10.3 mmol). The mixture was stirred at 25° C. for 2 hours. The reaction mixture was basified with saturated aqueous NaHCO₃ to pH=8, then extracted with DCM (30 mL×3), the combined organic layer was washed with brine (20 mL), dried over anhydrous Na₂SO₄, filtered and concentrated. The residue was purified by Combi Flash (0% to 100% EtOAc in PE) to give 6-bromoisoquinoline-4-carbaldehyde (1.20 g, yield: 79%) as a light yellow solid.

### Intermediate 29

### 5-ethynyl-1-methylpyridin-2(1H)-one

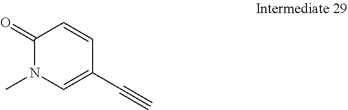

### Step 1. Synthesis of 1-methyl-5-((trimethylsilyl)ethynyl)pyridin-2(1H)-one

A mixture of 5-bromo-1-methylpyridin-2(1H)-one (2.00 g, 10.6 mmol), Pd(PPh₃)₂Cl₂ (746 mg, 1.06 mmol), CuI (405 mg, 2.13 mmol) and Et₃N (5.38 g, 53.19 mmol) in THF (20 mL) was degassed and purged with N₂ for 3 times at 0° C. Then ethynyltrimethylsilane (2.09 g, 21.3 mmol) was added into the reaction mixture and the mixture was stirred at 70° C. for 16 hours under N₂ atmosphere. The reaction mixture was concentrated and the residue was purified by flash silica gel chromatography (PE/EtOAc=1/1) to afford 1-methyl-5-((trimethylsilyl)ethynyl)pyridin-2(1H)-one (1.60 g, yield: 73%) as a brown solid.

¹H NMR (400 MHz, CDCl₃) δ 0.23 (9H, s), 3.53 (3H, s), 6.50 (1H, d, J=9.6 Hz), 7.35 (1H, dd, J=9.6, 2.4 Hz), 7.53 (1H, d, J=2.4 Hz).

### Step 2. Synthesis of 5-ethynyl-1-methylpyridin-2(1H)-one

A mixture of 1-methyl-5-((trimethylsilyl)ethynyl)pyridin-2(1H)-one (1.50 g, 7.31 mmol) in THF (20 mL) was added TBAF (14.6 mL, 14.6 mmol, 1M in THF) at 20° C. and stirred at 20° C. for 6 hours. The reaction mixture was quenched by addition saturated aqueous NH₄Cl (30 mL) and extracted with EtOAc (50 mL×3). The combined organic layer was washed with saturated aqueous NaHCO₃ (50 mL), H₂O (50 mL) and brine (50 mL), dried over anhydrous Na₂SO₄, filtered and concentrated. The residue was purified by flash silica gel chromatography (EtOAc as eluent) to afford 5-ethynyl-1-methylpyridin-2(1H)-one (600 mg, yield: 58%) as a yellow solid.

¹H NMR (400 MHz, CDCl₃) δ 3.02 (1H, s), 3.54 (3H, s), 6.52 (1H, d, J=9.6 Hz), 7.36 (1H, dd, J=9.2, 2.4 Hz), 7.55 (1H, d, J=2.4 Hz).

### Intermediate 30

### 4-methoxy-6-(4,4,5,5-tetramethyl-1,3,2-dioxaborolan-2-yl)isoquinoline

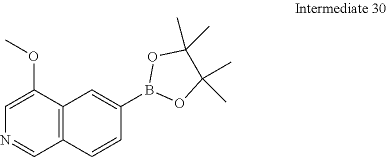 [SMILES: COc1cncc2ccc(B3OC(C)(C)C(C)(C)O3)cc12]

### Step 1. Synthesis of 6-bromo-4-methoxyisoquinoline

To a solution of compound Int-3 (50 mg, 0.22 mmol) in DCM (3 mL) and MeOH (0.75 mL) was added TMSCHN₂ (0.9 mL, 0.45 mmol, 2 M in hexane). The mixture was stirred at 20° C. for 2 hours. The reaction mixture was concentrated and the residue was purified by flash silica gel column (PE/EtOAc=1/2) to give 6-bromo-4-methoxyisoquinoline (25 mg, yield: 31%) as an off-white solid.

¹H NMR (400 MHz, DMSO-d₆) δ 4.06 (3H, s), 7.84-7.88 (1H, m), 8.09 (1H, d, J=8.8 Hz), 8.22-8.31 (2H, m), 8.98 (1H, s).

### Step 2. Synthesis of 4-methoxy-6-(4,4,5,5-tetramethyl-1,3,2-dioxaborolan-2-yl)isoquinoline

A mixture of 6-bromo-4-methoxyisoquinoline (100 mg, 0.420 mmol), Bis-Pin (213 mg, 0.840 mmol), KOAc (103 mg, 1.05 mmol) and Pd(dppf)Cl₂ (31 mg, 0.042 mmol) in dioxane (8 mL) was degassed and purged with N₂ for 3 times, and then the mixture was stirred at 90° C. for 16 hours under N₂ atmosphere. The reaction mixture was filtered and the filtrate was concentrated to give 4-methoxy-6-(4,4,5,5-tetramethyl-1,3,2-dioxaborolan-2-yl)isoquinoline (390 mg, crude) as a black oil.

### Intermediate 31

### tert-butyl 4-((6-bromoisoquinolin-3-yl)oxy)piperidine-1-carboxylate

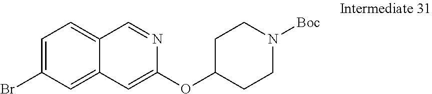 [SMILES: CC(C)(C)OC(=O)N1CCC(Oc2cc3cc(Br)ccc3cn2)CC1]

A mixture of 6-bromoisoquinolin-3-ol (1.00 g, 4.46 mmol), tert-butyl 4-hydroxypiperidine-1-carboxylate (1.80 g, 8.93 mmol), TMAD (2.31 g, 13.4 mmol) and n-Bu₃P (2.71 g, 13.4 mmol) in toluene (20 mL) was degassed and purged with N₂ for 3 times, and then the mixture was stirred at 110° C. for 16 hours under N₂ atmosphere. The reaction mixture was concentrated and the residue was purified by silica gel column (PE/EtOAc=5/1) to afford tert-butyl 4-((6-bromoisoquinolin-3-yl)oxy)piperidine-1-carboxylate (1.35 g, yield: 74%) as a yellow solid.

### Intermediate 32

### methyl 6-bromoisoquinoline-3-carboxylate

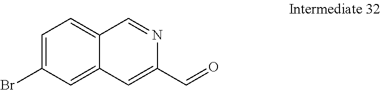 [SMILES: O=Cc1cc2cc(Br)ccc2cn1]

### Step 1. Synthesis of 6-bromoisoquinoline-3-carboxylic acid

The solution of 6-bromoisoquinoline-3-carboxylic acid (800 mg, 3.17 mmol) in DCM (24 mL) and MeOH (6 mL) was added TMSCHN₂ (3.2 mL, 6.40 mmol, 2M in hexane) slowly and stirred at 25° C. for 16 hours under N₂ atmosphere. The reaction mixture was concentrated and the residue was purified by silica gel column (PE/EtOAc=1/1) to afford 6-bromoisoquinoline-3-carboxylic acid (800 mg, yield: 76%) as a yellow solid.

¹H NMR (400 MHz, DMSO-d₆) δ 3.93 (3H, s), 8.00 (1H, dd, J=8.4, 2.0 Hz), 8.22 (1H, d, J=8.8 Hz), 8.55 (1H, d, J=1.6 Hz), 8.64 (1H, s), 9.43 (1H, s).

### Step 2. Synthesis of methyl 6-bromoisoquinoline-3-carboxylate

To a solution of methyl 6-bromoisoquinoline-3-carboxylate (800 mg, 3.01 mmol) in anhydrous toluene (18 mL) was added DIBAL-H (6.0 mL, 6.00 mmol, 1M in toluene) dropwise at −78° C. After the completion of the addition, the reaction mixture was stirred at −78° C. for 0.25 hour under N₂ atmosphere. The reaction mixture was quenched with MeOH (8 mL) dropwise at −78° C. and stirred at −78° C. for 0.5 hour under N₂ atmosphere. The mixture was diluted with H₂O (30 mL) and extracted with EtOAc (30 mL×3). The combined organic layers were dried over anhydrous Na₂SO₄, filtered and concentrated. The residue was purified by silica gel column (PE/EtOAc=3/1) to afford methyl 6-bromoisoquinoline-3-carboxylate (300 mg, yield: 42%) as a yellow solid.

¹H NMR (400 MHz, DMSO-d₆) δ 8.04 (1H, dd, J=8.8, 2.0 Hz), 8.25 (1H, d, J=8.8 Hz), 8.50 (1H, s), 8.59 (1H, d, J=1.6 Hz), 9.53 (1H, s), 10.16 (1H, s).

### Intermediate 33

### 4-((6-(4,4,5,5-tetramethyl-1,3,2-dioxaborolan-2-yl)isoquinolin-3-yl)oxy)cyclohexan-1-one

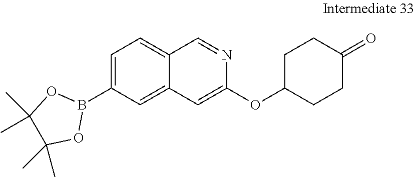 [SMILES: CC1(C)OB(c2ccc3cnc(OC4CCC(=O)CC4)cc3c2)OC1(C)C]

### Step 1. Synthesis of 3-((1,4-dioxaspiro[4.5]decan-8-yl)oxy)-6-bromoisoquinoline

A mixture of 6-bromoisoquinolin-3-ol (1.00 g, 4.46 mmol), 1,4-dioxaspiro[4.5]decan-8-ol (1.06 g, 6.69 mmol), PPh₃ (1.76 g, 6.69 mmol) in THF (10 mL) was degassed and purged with N₂ for 3 times, then DIAD (1.35 g, 6.69 mmol) was added to the reaction mixture at 0° C. and stirred at 80° C. for 4 hours under N₂ atmosphere. The reaction mixture was concentrated and the residue was purified by silica gel column (PE/EtOAc=5/1) to afford 3-((1,4-dioxaspiro[4.5]decan-8-yl)oxy)-6-bromoisoquinoline (1.20 g, yield: 74%) as a white solid.

### Step 2. Synthesis of 4-((6-bromoisoquinolin-3-yl)oxy)cyclohexan-1-one

To a solution of 3-((1,4-dioxaspiro[4.5]decan-8-yl)oxy)-6-bromoisoquinoline (1.10 g, 3.02 mmol) in dioxane (12 mL) was added 6N aqueous HCl (5 mL) at 20° C. The reaction mixture was stirred at 20° C. for 16 hours. The reaction mixture was concentrated and the residue was diluted with H₂O (30 mL), then extracted with DCM (40 mL×3). The combined organic layer was dried over anhydrous Na₂SO₄, filtered and concentrated. The residue was purified by silica gel column (PE/EtOAc=5/1) to afford 4-((6-bromoisoquinolin-3-yl)oxy)cyclohexan-1-one (900 mg, yield: 93%) as a white solid.

### Step 3. Synthesis of 4-((6-(4,4,5,5-tetramethyl-1,3,2-dioxaborolan-2-yl)isoquinolin-3-yl)oxy)cyclohexan-1-one

A mixture of 4-((6-bromoisoquinolin-3-yl)oxy)cyclohexan-1-one (460 mg, 1.44 mmol), Bis-Pin (730 mg, 2.87 mmol), Pd(dppf)Cl₂ (105 mg, 0.144 mmol) and KOAc (282 mg, 2.87 mmol) in dioxane (7 mL) was degassed and purged with N₂ for 3 times, then the mixture was stirred at 90° C. for 1.5 hours under N₂ atmosphere. The reaction mixture was concentrated and the residue was purified by silica gel column (PE/EtOAc=2/1) to afford 4-((6-(4,4,5,5-tetramethyl-1,3,2-dioxaborolan-2-yl)isoquinolin-3-yl)oxy)cyclohexan-1-one (510 mg, yield: 97%) as a white solid.

¹H NMR (400 MHz, DMSO-d₆) δ 1.34 (12H, s), 2.02-2.14 (2H, m), 2.16-2.29 (2H, m), 2.40-2.46 (4H, m), 5.39-5.50 (1H, m), 7.34 (1H, s), 7.62 (1H, d, J=8.0 Hz), 8.01 (1H, d, J=8.0 Hz), 8.21 (1H, s), 9.10 (1H, s).

### Intermediate 34

### 3-chloro-6-(4,4,5,5-tetramethyl-1,3,2-dioxaborolan-2-yl)isoquinoline

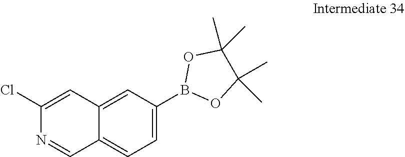 [SMILES: CC1(C)OB(c2ccc3cnc(Cl)cc3c2)OC1(C)C]

A mixture of compound 6-bromo-3-chloro-1,2-dihydroisoquinoline (1.00 g, 4.12 mmol), Bis-Pin (1.26 g, 4.95 mmol), Pd(dppf)Cl₂ (302 mg, 0.410 mmol) and KOAc (809 mg, 8.25 mmol) in 1, 4-dioxane (10 mL) was degassed and purged with N₂ for 3 times. Then the reaction mixture was stirred at 110° C. for 5 hours under N₂ atmosphere. The reaction mixture was concentrated and purified by silica gel column (PE/EtOAc=5/1) to afford 3-chloro-6-(4,4,5,5-tetramethyl-1,3,2-dioxaborolan-2-yl)isoquinoline (1.14 g, yield: 95%) as a white solid.

¹H NMR (400 MHz, DMSO-d₆) δ 1.34 (12H, s), 7.88 (1H, dd, J=8.4 Hz, 0.8 Hz), 8.14-8.16 (2H, m), 8.36 (1H, s), 9.24 (1H, s).

### Intermediate 35

### 3-cyclopropyl-6-(4,4,5,5-tetramethyl-1,3,2-dioxaborolan-2-yl)isoquinoline

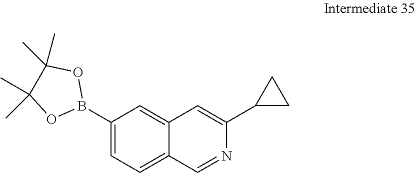 [SMILES: CC1(C)OB(c2ccc3cnc(C4CC4)cc3c2)OC1(C)C]

### Step 1. Synthesis of 2-(cyclopropylethynyl)-4-methoxybenzaldehyde

A mixture of 2-bromo-4-methoxybenzaldehyde (3.80 g, 17.7 mmol), Pd(PPh₃)₂Cl₂ (1.24 g, 1.77 mmol), CuI (1.01 g, 5.30 mmol) and Et₃N (12.30 mL) in THF (100 mL) was degassed and purged with N₂ for 3 times, then cyclopropylacetylene (5.13 mL, 61.9 mmol) was added and the reaction mixture was stirred at 60° C. for 12 hours under N₂ atmosphere. The reaction mixture was concentrated and the residue was purified by silica gel column (PE/EtOAc=10/1) to afford 2-(cyclopropylethynyl)-4-methoxybenzaldehyde (2.60 g, yield: 34%) as yellow oil.

¹H NMR (400 MHz, DMSO-d₆) δ 0.81-0.87 (2H, m), 0.92-0.98 (2H, m), 1.53-1.72 (1H, m), 3.85 (3H, s), 7.01-7.07 (2H, m), 7.73-7.78 (1H, m), 10.20 (1H, s).

### Step 2. Synthesis of 3-cyclopropyl-6-methoxyisoquinoline

To a solution of 2-(cyclopropylethynyl)-4-methoxybenzaldehyde (1.60 g, 7.99 mmol) in EtOH (20 mL) was added K₂CO₃ (11.0 g, 80.0 mmol) and 28% aq. NH₃·H₂O (5.60 g, 160 mmol). The mixture was stirred at 78° C. for 32 hours. The reaction mixture was concentrated and the residue was diluted with water (30 mL), then extracted with EtOAc (30 mL×3). The combined organic layers were dried over anhydrous Na₂SO₄, filtered and concentrated. The residue was purified by silica gel column (PE/EtOAc=5/1) to afford 3-cyclopropyl-6-methoxyisoquinoline (440 mg, yield: 28%) as a yellow solid.

¹H NMR (400 MHz, DMSO-d₆) δ 0.93-1.00 (4H, m), 2.11-2.21 (1H, m), 3.89 (3H, s), 7.15 (1H, dd, J=8.8, 2.0 Hz), 7.19 (1H, d, J=1.6 Hz), 7.56 (1H, s), 7.91 (1H, d, J=8.8 Hz), 8.99 (1H, s).

### Step 3. Synthesis of 3-cyclopropylisoquinolin-6-ol

A mixture of 3-cyclopropyl-6-methoxyisoquinoline (560 mg, 2.81 mmol) and pyridine hydrochloride (15 g, 126 mmol) was stirred at 200° C. for 3 hours. The reaction mixture was basified with 2N aqueous NaOH to pH=10 and diluted with water (30 mL), then extracted with EtOAc (30 mL×3). The combined organic layers were dried over anhydrous Na₂SO₄, filtered and concentrated. The residue was purified by silica gel column (PE/EtOAc=1/1) to afford 3-cyclopropylisoquinolin-6-ol (370 mg, yield: 71%) as a yellow solid.

### Step 4. Synthesis of 3-cyclopropylisoquinolin-6-yl trifluoromethanesulfonate

To a solution of 3-cyclopropylisoquinolin-6-ol (370 mg, 2.00 mmol) in DMF (8 mL) was added DIPEA (2.09 mL) and PhNTf₂ (856 mg, 2.40 mmol). The mixture was stirred at 25° C. for 1 hour. The reaction mixture was concentrated and the residue was purified by silica gel column (PE/EtOAc=10/1) to afford 3-cyclopropylisoquinolin-6-yl trifluoromethanesulfonate (600 mg, yield: 76%) as yellow oil.

### Step 5. Synthesis of 3-cyclopropyl-6-(4,4,5,5-tetramethyl-1,3,2-dioxaborolan-2-yl)isoquinoline

A mixture of 3-cyclopropylisoquinolin-6-yl trifluoromethanesulfonate (500 mg, 1.58 mmol), Bis-Pin (480 mg, 1.89 mmol), Pd(dppf)Cl₂ (115 mg, 0.158 mmol) and KOAc (464 mg, 4.73 mmol) in 1, 4-dioxane (15 mL) was degassed and purged with N₂ for 3 times, then the mixture was stirred at 90° C. for 16 hours under N₂ atmosphere. The reaction mixture was diluted with dioxane (35 mL) and filtered, the filtrate was concentrated to give 3-cyclopropyl-6-(4,4,5,5-tetramethyl-1,3,2-dioxaborolan-2-yl)isoquinoline (1.00 g, crude) as a black solid.

### Intermediate 36

### 3-chloro-6-(1-methyl-1H-pyrazol-4-yl)isoquinoline

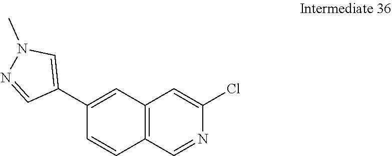 [SMILES: Cn1cc(-c2ccc3cnc(Cl)cc3c2)cn1]

A mixture of 6-bromo-3-chloroisoquinoline (1.00 g, 4.12 mmol), 1-methyl-4-(4,4,5,5-tetramethyl-1,3,2-dioxaborolan-2-yl)-1H-pyrazole (1.03 g, 4.95 mmol), Pd(dppf)Cl₂ (301 mg, 0.412 mmol) and Na₂CO₃ (874 mg, 8.25 mmol) in 1, 4-dioxane (20 mL) and H₂O (2 mL) was degassed and purged with N₂ for 3 times. Then the resulting mixture was stirred at 100° C. for 20 hours under N₂ atmosphere. The reaction mixture was diluted with H₂O (50 mL) and extracted with DCM (70 mL×3). The combined organic layers were washed with brine (70 mL), dried over anhydrous Na₂SO₄, filtered and concentrated. The residue was purified by silica gel column (PE/EtOAc=1/1) to afford 3-chloro-6-(1-methyl-1H-pyrazol-4-yl)isoquinoline (701 mg, yield: 69%) as a yellow gum.

### Compounds of Formula (I)

### Example 1

### 6-(imidazo[1,2-a]pyridin-3-yl)isoquinoline

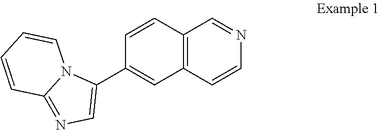 [SMILES: c1ccn2c(-c3ccc4cnccc4c3)cnc2c1]

A mixture of compound Int-1 (416 mg, 2.40 mmol), 3-bromoimidazo[1,2-a]pyridine (450 mg, 2.28 mmol), Pd(dppf)Cl₂·CH₂Cl₂ (98 mg, 0.12 mmol) and K₂CO₃ (997 mg, 7.21 mmol) in dioxane (4 mL) and H₂O (1 mL) was degassed and purged with N₂ for 3 times. Then the reaction mixture was stirred at 100° C. for 10 hours under N₂ atmosphere. The reaction mixture was filtered through a pad of celite and the filtrate was diluted with water (20 mL), then extracted with EtOAc (10 mL×5). The combined organic layer was washed with brine (10 mL×6), dried over anhydrous Na₂SO₄, filtered and concentrated. The residue was purified by prep-HPLC (0.05% HCOONH₄ as an additive; Method D), then lyophilized to give the title compound (8.0 mg, yield: 1.4%) as a pale yellow solid.

¹H NMR (400 MHz, CDCl₃) δ 6.79-7.06 (1H, m), 7.28-7.38 (1H, m), 7.43-8.26 (6H, m), 8.45-8.70 (2H, m), 9.31 (1H, s).

The following compounds were synthesized analogously to Example 1

| Example | | | |
|---|---|---|---|
| No. | Structure | Name | ¹H NMR (400 MHz) |
| 3 | 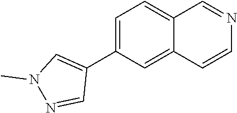 [SMILES: Cn1cc(-c2ccc3cnccc3c2)cn1] | 6-(1-methyl-1H- pyrazol-4-yl)- isoquinoline | CDCl₃; δ 4.00 (3H, s), 7.62 (1H, d, J = 5.6 Hz), 7.74 (1H, dd, J = 8.4, 1.6 Hz), 7.79 (1H, s), 7.88 (1H, s), 7.93 (1H, s), 7.97 (1H, d, J = 8.4 Hz), 8.51 (1H, d, J = 5.6 Hz), 9.20 (1H, s). |
| 60 | 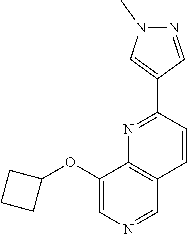 [SMILES: Cn1cc(-c2ccc3cncc(OC4CCC4)c3n2)cn1] | 8-cyclobutoxy-2- (1-methyl-1H- pyrazol-4-yl)- 1,6-naphthyridine | DMSO-d₆; δ 1.64-1.77 (1H, m), 1.82-1.92 (1H, m), 2.18-2.30 (2H, m), 2.54-2.62 (2H, m), 3.95 (3H, s), 4.96-5.08 (1H, m), 7.99 (1H, d, J = 8.4 Hz), 8.17 (1H, s), 8.21 (1H, s), 8.45 (1H, d, J = 8.8 Hz), 8.52 (1H, s), 8.86 (1H, s). |
| 63 | 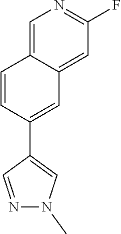 [SMILES: Cn1cc(-c2ccc3cnc(F)cc3c2)cn1] | 3-fluoro-6-(1- methyl-1H-pyr- azol-4-yl)iso- quinoline | DMSO-d₆; δ 3.91 (3H, s), 7.45 (1H, s), 7.88 (1H, dd, J = 8.8, 2.0 Hz), 8.07 (1H, s), 8.11-8.18 (2H, m), 8.38 (1H, s), 9.02 (1H, s). |
| 94 | 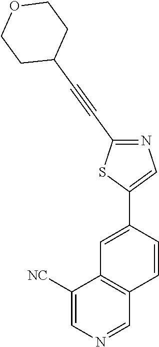 [SMILES: N#Cc1cncc2ccc(-c3cnc(C#CC4CCOCC4)s3)cc12] | 6-(2-((tetrahydro- 2H-pyran-4-yl)- ethynyl)thiazol- 5-yl)isoquinoline- 4-carbonitrile | DMSO-d₆; δ 1.61-1.73 (2H, m), 1.85-1.94 (2H, m), 2.97-3.17 (1H, m), 3.41-3.55 (2H, m), 3.76- 3.91 (2H, m), 8.14 (1H, s), 8.27 (1H, d, J = 8.4 Hz), 8.42 (1H, d, J = 8.4 Hz), 8.66 (1H, s), 9.11 (1H, s), 9.62 (1H, s). |

### Example 2

### 6-(1-methyl-1H-pyrazol-4-yl)-4-phenoxyisoquinoline

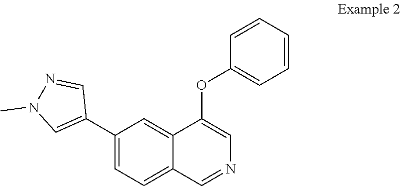 [SMILES: Cn1cc(-c2ccc3cncc(Oc4ccccc4)c3c2)cn1]

### Step 1. Synthesis of 6-bromo-4-phenoxyisoquinoline

To a solution of 6-bromo-4-iodoisoquinoline (100 mg, 0.299 mmol) and phenol (28 mg, 0.30 mmol) in DMSO (2 mL) was added CuI (6 mg, 0.03 mmol), K₃PO₄ (127 mg, 0.599 mmol) and 2-picolinic acid (7 mg, 0.06 mmol) under N₂ atmosphere. The mixture was stirred at 80° C. for 18 hours under N₂ atmosphere. The reaction mixture was diluted with EtOAc (20 mL) and saturated aqueous NaHCO₃ (20 mL) and separated. The aqueous phase was extracted with EtOAc (20 mL×2). The combined organic phase was washed with brine (30 mL), dried over anhydrous Na₂SO₄, filtered and concentrated under reduced pressure to give the residue. The residue was purified by column chromatography (SiO₂, eluent of 0˜23% ethyl acetate/petroleum ether gradient) to give 6-bromo-4-phenoxyisoquinoline (58 mg, yield: 65%) as a yellow solid.

¹H NMR (400 MHz, CDCl₃) δ 7.07-7.13 (2H, m), 7.17-7.24 (1H, m), 7.36-7.45 (2H, m), 7.73-7.79 (1H, m), 7.90 (1H, d, J=8.8 Hz), 8.12 (1H, s), 8.40 (1H, s), 8.93-9.11 (1H, m).

### Step 2. Synthesis of 6-(1-methyl-1H-pyrazol-4-yl)-4-phenoxyisoquinoline

A mixture of 6-bromo-4-phenoxyisoquinoline (133 mg, 0.443 mmol) and (1-methyl-1H-pyrazol-4-yl)boronic acid (67 mg, 0.53 mmol), Pd(PPh₃)₄ (51 mg, 0.044 mol) and Na₂CO₃ (94 mg, 0.89 mmol) in 1,4-dioxane (2 mL) and H₂O (0.5 mL) was degassed and purged with N₂ for 3 times. Then the mixture was stirred at 100° C. for 16 hours under N₂ atmosphere. The reaction mixture was poured into water (15 mL), and extracted with EtOAc (15 mL×3). The combined organic layer was washed with brine (20 mL), dried over anhydrous Na₂SO₄, filtered and concentrated under reduced pressure to give the residue. The residue was purified by prep-HPLC (0.04% NH₃H₂O+10 mM NH₄HCO₃ as an additive) and lyophilized to afford the title compound (40 mg, yield: 30%) as a light yellow oil.

¹H NMR (400 MHz, CD₃OD) δ 3.94 (3H, s), 7.10-7.16 (2H, m), 7.18-7.25 (1H, m), 7.40-7.47 (2H, m), 7.85 (1H, s), 7.93-7.98 (2H, m), 8.12 (1H, d, J=8.8 Hz), 8.15 (1H, s), 8.26 (1H, s), 8.92 (1H, s).

The following compounds were synthesized analogously to Example 2

| Example | | | |
|---|---|---|---|
| No. | Structure | Name | ¹H NMR (400 MHz) |
| 4 | 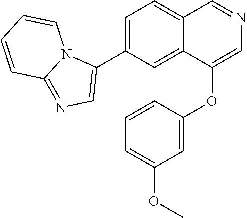 [SMILES: COc1cccc(Oc2cncc3ccc(-c4cnc5ccccn45)cc23)c1] | 6-(imidazo[1,2-a] pyridin-3-yl)-4-(3- methoxyphenoxy)- isoquinoline | CD₃OD; δ 3.78 (3H, s), 6.64 (1H, dd, J = 8.0, 1.6 Hz), 6.72-6.75 (1H, m), 6.81 (1H, dd, J = 8.4, 2.4 Hz), 6.92-6.99 (1H, m), 7.28-7.35 (1H, m), 7.37-7.44 (1H, m), 7.66 (1H, d, J = 8.8 Hz), 7.88-7.90 (1H, m), 7.98-8.04 (1H, m), 8.11 (1H, s), 8.28-8.34 (2H, m), 8.38 (1H, d, J = 6.8 Hz), 9.10 (1H, s). |
| 7 | 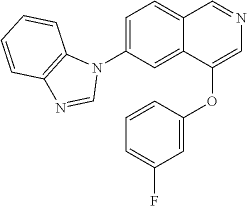 [SMILES: Fc1cccc(Oc2cncc3ccc(-n4cnc5ccccc54)cc23)c1] | 6-(1H-benzo[d] imidazol-1-yl)-4- (3-fluorophenoxy)- isoquinoline | CDCl₃; δ 6.80-6.85 (1H, m), 6.87-6.95 (2H, m), 7.32-7.42 (3H, m), 7.49-7.54 (1H, m), 7.88 (1H, dd, J = 8.8, 2.0 Hz), 7.90-7.94 (1H, m), 8.22-8.24 (1H, m), 8.25 (1H, s), 8.29 (1H, d, J = 8.8 Hz), 8.32 (1H, s), 9.21 (1H, s). |
| 8 | 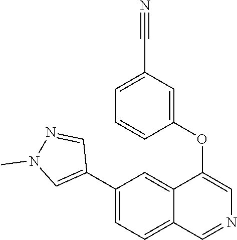 [SMILES: Cn1cc(-c2ccc3cncc(Oc4cccc(C#N)c4)c3c2)cn1] | 3-((6-(1-methyl- 1H-pyrazol-4-yl)- isoquinolin-4-yl)- oxy)benzonitrile | CDCl₃; δ 3.98 (3H, s), 7.30- 7.35 (2H, m), 7.41-7.52 (2H, m), 7.77 (1H, s), 7.82 (1H, dd, J = 8.8, 1.6 Hz), 7.88 (1H, s), 8.01-8.09 (2H, m), 8.18 (1H, s), 9.09 (1H, s). |
| 9 | 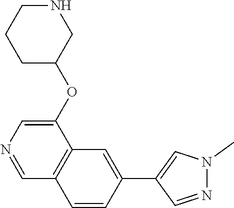 [SMILES: Cn1cc(-c2ccc3cncc(OC4CCCNC4)c3c2)cn1] | 6-(1-methyl-1H- pyrazol-4-yl)-4- (piperidin-3-yl- oxy)isoquinoline | DMSO-d₆; δ 1.50-1.68 (1H, m), 1.75-1.95 (2H, m), 1.97- 2.10 (1H, m), 2.84-2.97 (2H, m), 3.06-3.15 (1H, m), 3.25- 3.33 (1H, m), 3.91 (3H, s), 4.79-4.81 (1H, m), 7.93 (1H, dd, J = 8.8, 1.6 Hz), 8.06 (1H, d, J = 8.8 Hz), 8.12 (1H, s), 8.20 (1H, s), 8.31 (1H, s), 8.35 (1H, s), 8.44 (1H, s) 8.85 (1H, s). |
| 20 | 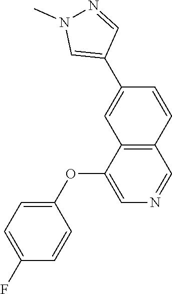 [SMILES: Cn1cc(-c2ccc3cncc(Oc4ccc(F)cc4)c3c2)cn1] | 4-(4-fluorophen- oxy)-6-(1-methyl- 1H-pyrazol-4-yl)- isoquinoline | CDCl₃; δ 3.99 (3H, s), 7.05- 7.12 (4H, m), 7.76-7.83 (2H, m), 7.89-7.94 (1H, m), 7.98- 8.04 (2H, m), 8.19-8.24 (1H, m), 8.97 (1H, s). |
| 23 | 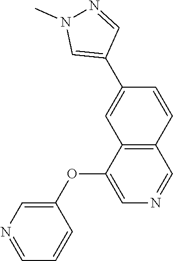 [SMILES: Cn1cc(-c2ccc3cncc(Oc4cccnc4)c3c2)cn1] | 6-(1-methyl-1H- pyrazol-4-yl)-4- (pyridin-3-yloxy)- isoquinoline | DMSO-d₆; δ 3.88 (3H, s), 7.41-7.47 (1H, m), 7.49-7.55 (1H, m), 8.03 (1H, dd, J = 8.8, 1.6 Hz), 8.05-8.08 (1H, m), 8.11 (1H, s) 8.14-8.17 (1H, m), 8.22 (1H, d, J = 8.4 Hz), 8.39-8.42 (2H, m), 8.55 (1H, d, J = 2.8 Hz), 9.11 (1H, s). |
| 24 | 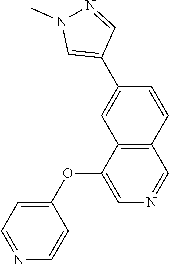 [SMILES: Cn1cc(-c2ccc3cncc(Oc4ccncc4)c3c2)cn1] | 6-(1-methyl-1H- pyrazol-4-yl)-4- (pyridin-4-yloxy)- isoquinoline | CD₃OD; δ 3.93 (3H, s), 7.06- 7.11 (2H, m), 7.94 (1H, s), 7.99-8.04 (2H, m), 8.15 (1H, s), 8.23 (1H, d, J = 9.2 Hz), 8.29 (1H, s), 8.44-8.50 (2H, m), 9.15 (1H, s). |
| 41 | 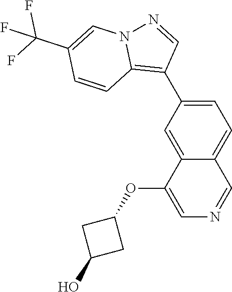 [SMILES: O[C@H]1C[C@H](Oc2cncc3ccc(-c4cnn5cc(C(F)(F)F)ccc45)cc23)C1] | (1r,3r)-3-((6-(6- (trifluoromethyl)- pyrazolo[1,5-a]pyr- idin-3-yl)isoquin- olin-4-yl)oxy)- cyclobutan-1-ol | DMSO-d₆; δ 2.39-2.47 (2H, m), 2.51-2.57 (2H, m), 4.43- 4.53 (1H, m), 5.10-5.18 (1H, m), 5.26 (1H, d, J = 5.2 Hz), 7.66 (1H, dd, J = 9.2, 1.6 Hz), 7.93 (1H, s), 8.09 (1H, dd, J = 8.4, 1.6 Hz), 8.18-8.24 (2H, m), 8.32-8.36 (1H, m), 8.83 (1H, s), 8.94 (1H, s), 9.48 (1H, s). |
| 52 | 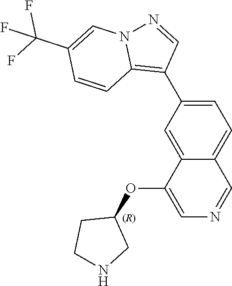 | (R)-4-(pyrrolidin- 3-yloxy)-6-(6- (trifluoromethyl)- pyrazolo[1,5-a]pyr- idin-3-yl)isoquino- line | DMSO-d₆; δ 2.34-2.41 (2H, m), 3.60-3.73 (4H, m), 5.48- 5.58 (1H, m), 7.66 (1H, dd, J = 9.6, 1.2 Hz), 8.20 (1H, dd, J = 8.4, 1.6 Hz), 8.27- 8.35 (3H, m), 8.46 (1H, s), 8.86 (1H, s), 8.98-9.08 (1H, m), 9.12 (1H, brs), 9.16-9.28 (1H, m), 9.52 (1H, s). |
| 53 | 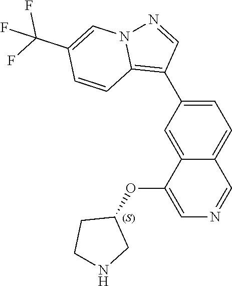 | (S)-4-(pyrrolidin- 3-yloxy)-6-(6- (trifluoromethyl)- pyrazolo[1,5-a]pyr- idin-3-yl)isoquino- line | DMSO-d₆; δ 2.34-2.43 (2H, m), 3.39-3.58 (4H, m), 5.51- 5.57 (1H, m), 7.67 (1H, dd, J = 9.6, 1.6 Hz), 8.23 (1H, dd, J = 8.4, 1.6 Hz), 8.29-8.36 (3H, m), 8.48 (1H, s), 8.88 (1H, s), 9.08-9.22 (2H, m), 9.24-9.40 (1H, m), 9.52 (1H, s). |
| 70 | 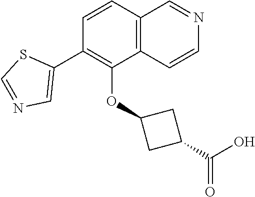 [SMILES: O=C(O)[C@H]1C[C@H](Oc2c(-c3cncs3)ccc3cnccc23)C1] | (1r,3r)-3-((6- (thiazol-5-yl)iso- quinolin-5-yl)oxy)- cyclobutane-1- carboxylic acid | DMSO-d₆; δ 2.29-2.36 (2H, m), 2.53-2.59 (2H, m), 2.88- 3.01 (1H, m), 4.45-4.79 (1H, m), 7.96 (1H, d, J = 5.6 Hz), 8.00 (1H, d, J = 8.4 Hz), 8.08 (1H, d, J = 8.4 Hz), 8.55-8.60 (2H, m), 9.26 (1H, s), 9.35 (1H, s), 12.28 (1H, brs). |
| 79 | 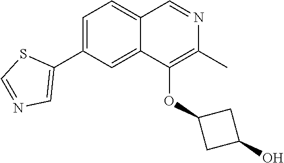 [SMILES: Cc1ncc2ccc(-c3cncs3)cc2c1O[C@H]1C[C@@H](O)C1] | (1s,3s)-3-((3-meth- yl-6-(thiazol-5-yl)- isoquinolin-4-yl)- oxy)cyclobutan-1- ol | DMSO-d₆; δ 2.17-2.27 (2H, m), 2.57 (3H, s), 2.67-2.77 (2H, m), 3.63-3.75 (1H, m), 4.04-4.18 (1H, m), 5.21 (1H, d, J = 6.4 Hz), 8.02 (1H, dd, J = 8.4, 1.6 Hz), 8.11 (1H, s), 8.19 (1H, d, J = 8.4 Hz), 8.57 (1H, s), 9.03 (1H, s), 9.22 (1H, s). |
| 80 | 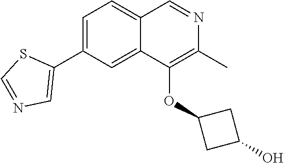 [SMILES: Cc1ncc2ccc(-c3cncs3)cc2c1O[C@H]1C[C@H](O)C1] | (1r,3r)-3-((3-meth- yl-6-(thiazol-5-yl)- isoquinolin-4-yl)- oxy)cyclobutan-1- ol | DMSO-d₆; δ 2.18-2.26 (2H, m), 2.53-2.56 (2H, m), 2.58 (3H, s), 4.46-4.63 (1H, m), 4.73-4.88 (1H, m), 5.05-5.23 (1H, m), 8.02 (1H, dd, J = 8.8, 1.6 Hz), 8.13 (1H, s), 8.19 (1H, d, J = 8.4 Hz), 8.58 (1H, s), 9.03 (1H, s), 9.22 (1H, s). |
| 81 | 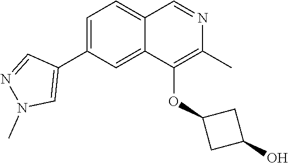 [SMILES: Cc1ncc2ccc(-c3cnn(C)c3)cc2c1O[C@H]1C[C@@H](O)C1] | (1s,3s)-3-((3-meth- yl-6-(1-methyl-1H- pyrazol-4-yl)iso- quinolin-4-yl)oxy)- cyclobutan-1-ol | DMSO-d₆; δ 2.16-2.28 (2H, m), 2.55 (3H, s), 2.65-2.78 (2H, m), 3.61-3.74 (1H, m), 3.93 (3H, s), 4.04-4.18 (1H, m), 5.18-5.20 (1H, m), 7.85 (1H, dd, J = 8.4, 1.6 Hz), 8.02 (1H, s), 8.03-8.09 (2H, m), 8.38 (1H, s), 8.92 (1H, s). |
| 82 | 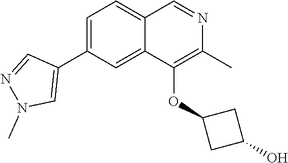 [SMILES: Cc1ncc2ccc(-c3cnn(C)c3)cc2c1O[C@H]1C[C@H](O)C1] | (1r,3r)-3-((3-meth- yl-6-(1-methyl-1H- pyrazol-4-yl)iso- quinolin-4-yl)oxy)- cyclobutan-1-ol | DMSO-d₆; δ 2.15-2.24 (2H, m), 2.52-2.54 (2H, m), 2.55 (3H, s), 3.93 (3H, s), 4.48- 4.60 (1H, m), 4.76-4.86 (1H, m), 4.95-5.27 (1H, m), 7.85 (1H, dd, J = 8.4, 1.6 Hz), 8.04 (1H, s), 8.05-8.10 (2H, m), 8.40 (1H, s), 8.93 (1H, s). |

### Example 5

### 3-((6-(imidazo[1,2-a]pyridin-3-yl)isoquinolin-4-yl)oxy)phenol

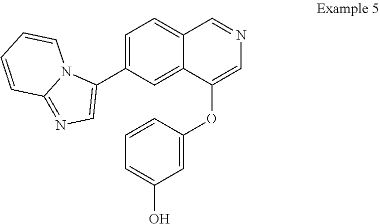 [SMILES: Oc1cccc(Oc2cncc3ccc(-c4cnc5ccccn45)cc23)c1]

Example 4 (60 mg, 0.16 mmol) was dissolved in dry DCM (2 mL). BBr₃ (614 mg, 2.45 mmol) was added to the mixture dropwise at 0° C. The solution was stirred at 0° C. for 2 hours. The reaction mixture was quenched with MeOH (3 mL) and poured into water (15 mL), extracted with DCM (15 mL×3). The combined organic layer washed with brine (20 mL), dried over anhydrous Na₂SO₄, filtered and concentrated to give the residue. The residue was purified by prep-HPLC (0.04% NH₃H₂O+10 mM NH₄HCO₃ as an additive) and lyophilized to give the title compound (8 mg, yield: 3% for two steps) as a light yellow solid.

¹H NMR (400 MHz, DMSO-d₆) δ 6.45-6.49 (1H, m), 6.53 (1H, dd, J=7.6, 1.6 Hz), 6.60 (1H, dd, J=8.4, 2.0 Hz), 6.90-6.97 (1H, m), 7.16-7.24 (1H, m), 7.33-7.39 (1H, m), 7.68-7.73 (1H, m), 8.00 (1H, s), 8.06 (1H, dd, J=8.8, 2.0 Hz), 8.12 (1H, s), 8.28 (1H, s), 8.34-8.40 (2H, m), 9.24 (1H, s), 9.67 (1H, brs).

### Example 6

### 3-((6-(imidazo[1,2-a]pyridin-3-yl)isoquinolin-4-yl)oxy)benzonitrile

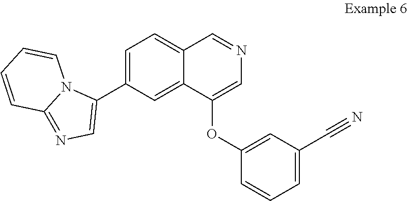 [SMILES: N#Cc1cccc(Oc2cncc3ccc(-c4cnc5ccccn45)cc23)c1]

### Step 1. Synthesis of 3-((6-(imidazo[1,2-a]pyridin-3-yl)isoquinolin-4-yl)oxy)phenyl trifluoromethanesulfonate

To a solution of Example 5 (90 mg, crude) and Et₃N (129 mg, 1.27 mmol) in DCM (3 mL) was added Tf₂O (180 mg, 0.637 mmol) at 0° C., the mixture was stirred at 0° C. for 2 hours. The reaction mixture was poured into water (15 mL) and extracted with DCM (15 mL×3). The combined organic layer was washed with brine (20 mL), dried over anhydrous Na₂SO₄, filtered and concentrated to give the residue. The residue was purified by flash silica gel chromatography (ISCO®; 4 g SepaFlash® Silica Flash Column, Eluent of 0˜3% methanol/dichloromethane gradient @20 mL/min) to give 3-((6-(imidazo[1,2-a]pyridin-3-yl)isoquinolin-4-yl)oxy)phenyl trifluoromethanesulfonate (110 mg, yield: 80% for two steps) as a yellow solid.

### Step 2. Synthesis of 3-((6-(imidazo[1,2-a]pyridin-3-yl)isoquinolin-4-yl)oxy)benzonitrile

To a solution of 3-((6-(imidazo[1,2-a]pyridin-3-yl)isoquinolin-4-yl)oxy)phenyl trifluoromethanesulfonate (90 mg, 0.19 mmol) and Zn(CN)₂ (120 mg, 1.02 mmol,) in DMF (2 mL) was added Pd(PPh₃)₄ (21 mg, 0.019 mol) under N₂ atmosphere. The mixture was stirred at 100° C. for 16 hours under N₂ atmosphere. The reaction mixture poured into H₂O (20 mL) and extracted with EtOAc (20 mL×3). The combined organic layer was washed with brine (30 mL), dried over anhydrous Na₂SO₄, filtered and concentrated under reduced pressure to give the residue. The residue was purified by prep-HPLC (0.05% NH₃H₂O+10 mM NH₄HCO₃ as an additive), then further purified by prep-TLC (DCM/MeOH=10/1) to give the title compound (14 mg, yield: 20%) as a white solid.

¹H NMR (400 MHz, CD₃OD) δ 6.97-7.03 (1H, m), 7.39-7.47 (2H, m), 7.54-7.57 (1H, m), 7.57-7.64 (2H, m), 7.65-7.70 (1H, m), 7.90 (1H, s), 8.07 (1H, dd, J=8.4, 1.2 Hz), 8.19 (1H, s), 8.30 (1H, s), 8.39 (1H, d, J=8.4 Hz), 8.49 (1H, d, J=7.2 Hz), 9.19 (1H, s).

### Example 10

### 6-(1-methyl-1H-pyrazol-4-yl)-4-((1-methylpiperidin-3-yl)oxy)isoquinoline

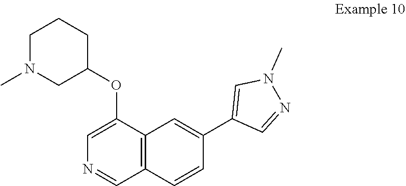 [SMILES: CN1CCCC(Oc2cncc3ccc(-c4cnn(C)c4)cc23)C1]

To a solution of Example 9 (260 mg, 0.843 mmol) and 37% aqueous formaldehyde (342 mg, 4.22 mmol) in MeOH (5 mL) was added HOAc (152 mg, 2.53 mmol), the mixture was stirred at 20° C. for 1 hour. NaBH₃CN (159 mg, 2.53 mmol) was added to the above reaction mixture, the mixture was stirred at 45° C. for 11 hours. The reaction mixture was diluted with H₂O (40 mL) and extracted with EtOAc (40 mL×3). The combined organic layer was dried over anhydrous Na₂SO₄, filtered and concentrated to give the residue. The residue was purified by prep-HPLC (0.05% NH₃H₂O+10 mM NH₄HCO₃ as an additive) and lyophilized to give the title compound (106 mg, yield: 41% for two steps) as a yellow solid.

¹H NMR (400 MHz, CDCl₃) δ 1.53-1.74 (2H, m), 1.87-1.98 (1H, m), 2.09-2.35 (3H, m), 2.36 (3H, s), 2.69-2.78 (1H, m), 3.13-3.21 (1H, m), 4.00 (3H, s), 4.61-4.71 (1H, m), 7.73 (1H, dd, J=8.4, 1.6 Hz), 7.81 (1H, s), 7.88-7.94 (2H, m), 8.16 (1H, s), 8.23 (1H, s) 8.83 (1H, s).

### Example 11

### N-(5-(isoquinolin-6-yl)thiazol-2-yl)-1-methylpiperidine-4-carboxamide

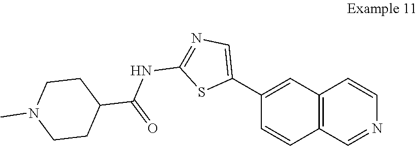 [SMILES: CN1CCC(C(=O)Nc2ncc(-c3ccc4cnccc4c3)s2)CC1]

### Step 1. Synthesis of N-(5-bromothiazol-2-yl)-1-methylpiperidine-4-carboxamide

A mixture of 5-bromothiazol-2-amine (200 mg, 1.12 mmol), 1-methylpiperidine-4-carboxylic acid (208 mg, 1.45 mmol), T₃P (2.13 g, 3.35 mmol, 50% in EtOAc), Et₃N (226 mg, 2.23 mmol) in pyridine (2 mL) was stirred at 50° C. for 16 hours. The reaction mixture was diluted with EtOAc (20 mL) and washed with saturated aqueous NaHCO₃ (30 mL×3), brine (20 mL), dried over anhydrous Na₂SO₄ and concentrated. The residue was purified by flash silica gel chromatography (ISCO®; 4 g SepaFlash® Silica Flash Column, Eluent of 0˜12% MeOH/DCM @25 mL/min) to give N-(5-bromothiazol-2-yl)-1-methylpiperidine-4-carboxamide (200 mg, yield: 59%) as a yellow solid.

¹H NMR (400 MHz, CD₃OD) δ 1.90-2.06 (4H, m), 2.40-2.49 (2H, m), 2.50 (3H, s), 2.55-2.65 (1H, m), 3.10-3.20 (2H, m), 7.40 (1H, s).

### Step 2. Synthesis of N-(5-(isoquinolin-6-yl)thiazol-2-yl)-1-methylpiperidine-4-carboxamide

A mixture of N-(5-bromothiazol-2-yl)-1-methylpiperidine-4-carboxamide (100 mg, 0.329 mmol), 6-(4,4,5,5-tetramethyl1,3,2-dioxaborolan-2-yl)isoquinoline (101 mg, 0.394 mmol), Pd(dppf)Cl₂ (24 mg, 0.033 mmol) and NaHCO₃ (83 mg, 0.99 mmol) in dioxane (3 mL) and water (1 mL) was degassed and purged with N₂ for 3 times. Then the mixture was stirred at 90° C. for 16 hours under N₂ atmosphere. The reaction mixture was filtered and the filtrate was concentrated. The residue was purified by flash silica gel chromatography (ISCO®; 12 g SepaFlash® Silica Flash Column, Eluent of 0˜15% MeOH/DCM @25 mL/min), then further purified by prep-HPLC (0.225% FA as an additive; Method C) to give the title compound (7.95 mg, yield: 12%, FA salt) as a yellow solid.

¹H NMR (400 MHz, DMSO-d₆) δ 1.61-1.74 (2H, m), 1.77-1.86 (2H, m), 1.85-1.95 (2H, m), 2.18 (3H, s), 2.40-2.45 (1H, m), 2.80-2.90 (2H, m), 7.84 (1H, d, J=6.0 Hz), 8.03 (1H, dd, J=8.4, 1.6 Hz), 8.11-8.17 (3H, m), 8.27 (1H, s), 8.50 (1H, d, J=5.6 Hz), 9.28 (s, 1H, brs).

The following compounds were synthesized analogously to Example 11

| Example | | | |
|---|---|---|---|
| No. | Structure | Name | ¹H NMR (400 MHz) |
| 14 | 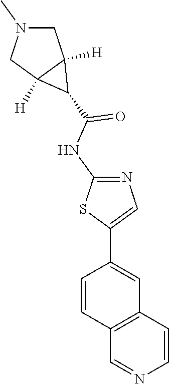 [SMILES: CN1C[C@@H]2[C@H](C1)[C@H]2C(=O)Nc1ncc(-c2ccc3cnccc3c2)s1] | (1R,5S,6r)-N-(5-(iso- quinolin-6-yl)thiazol- 2-yl)-3-methyl-3-aza- bicyclo[3.1.0]hexane- 6-carboxamide | D₂O; δ 2.00-2.05 (1H, m) 2.40- 2.45 (2H, m), 2.90 (3H, s), 3.45-3.50 (2H, m), 3.84 (2H, m), 7.85 (1H, s), 7.93-8.00 (2H, m), 8.05 (1H, d, J = 6.4 Hz), 8.18 (1H, d, J = 8.8 Hz), 8.26 (1H, d, J = 6.4 Hz), 9.29 (1H, s). |
| 34 | 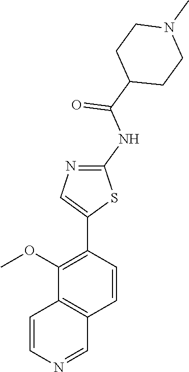 | N-(5-(5-methoxyiso- quinolin-6-yl)thiazol- 2-yl)-1-methylpiperi- dine-4-carboxamide | CD₃OD; δ 1.99-2.16 (2H, m), 2.21-2.32 (2H, m), 2.87-2.98 (4H, m), 3.07-3.18 (2H, m), 3.63-3.71 (2H, m), 4.01 (3H, s), 8.20 (1H, d, J = 8.4 Hz), 8.33 (1H, s), 8.39 (1H, d, J = 8.8 Hz), 8.46 (1H, d, J = 6.4 Hz), 8.58 (1H, d, J = 6.8 Hz), 9.57 (1H, s). |

### Example 12

### N-(5-(isoquinolin-6-yl)thiazol-2-yl)-1-methylazetidine-3-carboxamide

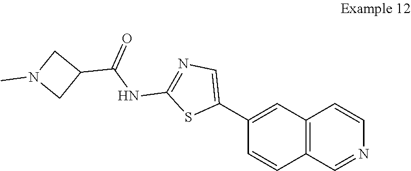 [SMILES: CN1CC(C(=O)Nc2ncc(-c3ccc4cnccc4c3)s2)C1]

### Step 1. Synthesis of tert-butyl 3-((5-(isoquinolin-6-yl)thiazol-2-yl)carbamoyl)azetidine-1-carboxylate

A mixture of compound Int-4 (100 mg, 0.440 mmol), 1-(tert-butoxycarbonyl)azetidine-3-carboxylic acid (133 mg, 0.660 mmol), T₃P (840 mg, 1.32 mmol, 50% in EtOAc), Et₃N (145 mg, 1.44 mmol) in pyridine (2 mL) was stirred at 50° C. for 1 hour. The reaction mixture was filtered and the filtrate was concentrated under reduced pressure. The residue was purified by flash silica gel chromatography (ISCO®; 4 g SepaFlash® Silica Flash Column, Eluent of 0˜14% MeOH/DCM @25 mL/min) to give tert-butyl 3-((5-(isoquinolin-6-yl)thiazol-2-yl)carbamoyl)azetidine-1-carboxylate (180 mg, yield: >99%) as a yellow solid.

### Step 2. Synthesis of N-(5-(isoquinolin-6-yl)thiazol-2-yl)azetidine-3-carboxamide

A mixture of tert-butyl 3-((5-(isoquinolin-6-yl)thiazol-2-yl)carbamoyl)azetidine-1-carboxylate (200 mg, 0.487 mmol) in DCM (5 mL) and TFA (1 mL) was stirred at 25° C. for 1 hour. The reaction mixture was concentrated to give N-(5-(isoquinolin-6-yl)thiazol-2-yl)azetidine-3-carboxamide (200 mg, crude, TFA salt) as a yellow gum, which was directly used for the next step without further purification.

### Step 3. Synthesis of N-(5-(isoquinolin-6-yl)thiazol-2-yl)-1-methylazetidine-3-carboxamide

To a solution of N-(5-(isoquinolin-6-yl)thiazol-2-yl)azetidine-3-carboxamide (200 mg, crude, TFA salt) in MeOH (5 mL) was added DIPEA to adjust the pH=5, then 37% aqueous HCHO (262 mg, 3.22 mmol) was added to the reaction mixture and stirred at 25° C. for 30 minutes. NaBH₃CN (121 mg, 1.93 mmol) was added and the resulting reaction mixture was stirred at 25° C. for another 2 hours. The reaction mixture was diluted with water (50 mL) and extracted with EtOAc (30 mL×3). The combined organic layer were washed with brine (30 mL×3), dried over anhydrous Na₂SO₄, filtered and concentrated. The residue was purified by flash silica gel chromatography (ISCO®; 4 g SepaFlash® Silica Flash Column, Eluent of 0˜15% MeOH/DCM @25 mL/min), then triturated with CH₃CN (5 mL) to give the title compound (50.28 mg, yield: 24%) as a yellow solid.

¹H NMR (400 MHz, DMSO-d₆) δ 2.40 (3H, s), 3.50-3.55 (2H, m), 3.57-3.62 (1H, m), 3.65-3.70 (2H, m), 7.86 (1H, d, J=5.6 Hz), 8.05-8.10 (1H, m), 8.15-8.20 (3H, m), 8.51 (1H, d, J=6.0 Hz), 9.28 (1H, s).

The following compounds were synthesized analogously to Example 12

| Example | | | |
|---|---|---|---|
| No. | Structure | Name | ¹H NMR (400 MHz) |
| 13 | 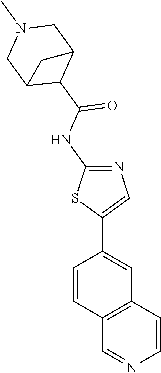 [SMILES: CN1CC2CC(C1)C2C(=O)Nc1ncc(-c2ccc3cnccc3c2)s1] | N-(5-(isoquinolin-6- yl)thiazol-2-yl)-3- methyl-3- azabicyclo[3.1.1]hep- tane-6-carboxamide | CD₃OD; δ 1.46-1.74 (1H, m), 2.25-2.30 (1H, m), 2.57-2.73 (3H, m), 2.77-2.88 (2H, m), 2.90-3.30 (3H, m), 3.42-3.66 (2H, m), 7.86 (1H, d, J = 5.6 Hz), 7.97- 8.05 (2H, m), 8.10-8.18 (2H, m), 8.45 (1H, d, J = 5.6 Hz, 1H), 9.21 (1H, s). |
| 18 | 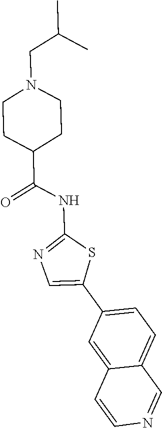 [SMILES: CC(C)CN1CCC(C(=O)Nc2ncc(-c3ccc4cnccc4c3)s2)CC1] | 1-isobutyl-N-(5- (isoquinolin-6- yl)thiazol-2- yl)piperidine-4- carboxamide | DMSO-d₆; δ 0.87 (6H, d, J = 6.4 Hz), 1.63-1.76 (2H, m), 1.77-1.89 (3H, m), 1.90-2.00 (2H, m), 2.10-2.15 (2H, m), 2.50-2.52 (1H, m) 2.90-2.95 (2H, m), 7.84 (1H, d, J = 5.6 Hz), 8.03 (1H, dd, J = 8.4, 1.6 Hz), 8.13-8.16 (3H, m), 8.50 (1H, d, J = 5.6 Hz), 9.28 (1H, s), 12.31 (1H, brs). |
| 19 | 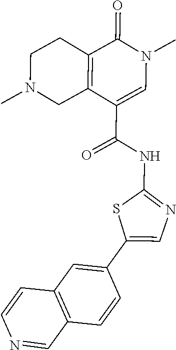 | N-(5-(isoquinolin-6- yl)thiazol-2-yl)-2,6- dimethyl-1-oxo- 1,2,5,6,7,8- hexahydro-2,6- naphthyridine-4- carboxamide | DMSO-d₆: δ 2.35 (3H, s), 2.56 (2H, d, J = 5.6 Hz), 3.48-3.52 (5H, m), 3.55-3.65 (2H, m), 7.82-7.88 (1H, m), 8.04-8.10 (1H, m), 8.14 (1H, d, J = 1.6 Hz), 8.15- 8.20 (1H, m), 8.22 (1H, s), 8.34 (1H, s), 8.50 (1H, d, J = 6.0 Hz), 9.28 (1H, s). |

### Example 15

### N-(5-(isoquinolin-6-yl)thiazol-2-yl)-2-methylnicotinamide

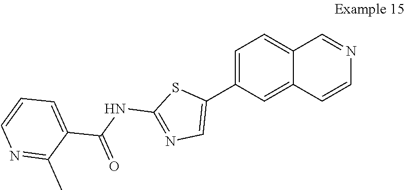 [SMILES: Cc1ncccc1C(=O)Nc1ncc(-c2ccc3cnccc3c2)s1]

A mixture of compound Int-4 (100 mg, 0.440 mmol), 2-methylpyridine-3-carboxylic acid (72 mg, 0.53 mmol) and EDCI (169 mg, 0.880 mmol) in pyridine (5 mL) was stirred at 90° C. for 1 hour. The reaction mixture was turned into red solution from yellow suspension. The mixture was diluted with saturated aqueous NaHCO₃ (30 mL) and extracted with DCM/MeOH (30 mL×3, 10/1). The combined organic layer was washed with brine (30 mL), dried over anhydrous Na₂SO₄ and concentrated. The residue was purified by prep-HPLC (0.2250 FA as an additive; Method C) and lyophilized to give the title compound (43 mg, yield: 270%) as a yellow solid.

¹H NMR (400 MHz, DMSO-d₆) δ 2.58 (3H, s), 7.46 (1H, d, J=8.0 Hz), 7.87 (1H, d, J=5.6 Hz), 8.07-8.11 (1H, m), 8.16 (1H, d, J=4.8 Hz), 8.18-8.21 (1H, m), 8.27 (1H, s), 8.35 (1H, dd, J=8.0, 2.4 Hz), 8.52 (1H, d, J=5.6 Hz), 9.15 (1H, d, J=2.0 Hz), 9.29 (1H, s), 12.93 (1H, brs).

The following compounds were synthesized analogously to Example 15

| Example | | | |
|---|---|---|---|
| No. | Structure | Name | ¹H NMR (400 MHz) |
| 16 | 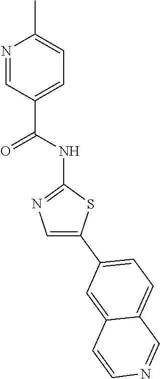 [SMILES: Cc1ccc(C(=O)Nc2ncc(-c3ccc4cnccc4c3)s2)cn1] | N-(5-(isoquinolin- 6-yl)thiazol-2-yl)- 6- methylnicotinamide | DMSO-d₆; δ 2.63 (3H, s), 7.39 (1H, dd, J = 8.0, 4.8 Hz), 7.87 (1H, d, J = 5.6 Hz), 8.03 (1H, dd, J = 8.0, 1.6 Hz), 8.07-8.12 (1H, m), 8.16-8.20 (1H, m), 8.23 (1H, s), 8.25 (1H, s), 8.52 (1H, d, J = 5.6 Hz), 8.61 (1H, d, J = 3.2 Hz), 9.30 (1H, s), 12.86 (1H, brs). |
| 17 | 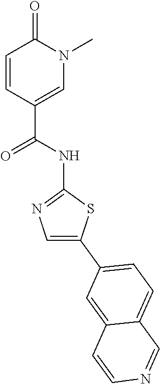 [SMILES: Cn1cc(C(=O)Nc2ncc(-c3ccc4cnccc4c3)s2)ccc1=O] | N-(5-(isoquinolin- 6-yl)thiazol-2-yl)- 1-methyl-6-oxo- 1,6- dihydropyridine-3- carboxamide | DMSO-d₆; δ 3.54 (3H, s), 6.50 (1H, d, J = 9.6 Hz), 7.86 (1H, d, J = 5.6 Hz), 8.04-8.10 (2H, m), 8.15 (1H, d, J = 2.4 Hz), 8.17 (1H, s), 8.23 (1H, s), 8.51 (1H, d, J = 5.6 Hz), 8.77 (1H, d, J = 2.4 Hz), 9.29 (1H, s), 12.61 (1H, brs). |
| 28 | 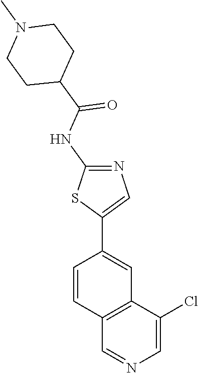 [SMILES: CN1CCC(C(=O)Nc2ncc(-c3ccc4cncc(Cl)c4c3)s2)CC1] | N-(5-(4- chloroisoquinolin- 6-yl)thiazol-2-yl)- 1-methylpiperidine- 4-carboxamide | DMSO-d₆; δ 1.70-1.75 (2H, m), 1.80- 1.85 (2H, m), 2.01-2.07 (2H, m), 2.27 (3H, s), 2.90-2.95 (3H, m), 8.12 (1H, s), 8.15-8.22 (3H, m), 8.25-8.32 (2H, m), 8.65 (1H, s), 9.28 (1H, s). |
| 29 | 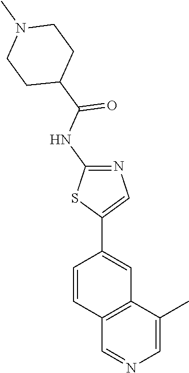 | 1-methyl-N-(5-(4- methylisoquinolin- 6-yl)thiazol-2- yl)piperidine-4- carboxamide | DMSO-d₆; δ 1.62-1.75 (2H, m), 1.78- 1.87 (2H, m), 1.88-1.97 (2H, m), 2.19 (3H, s), 2.52-2.54 (1H, m), 2.63 (3H, s), 2.80-2.89 (2H, m), 8.01 (1H, dd, J = 8.4, 1.6 Hz), 8.07 (1H, s), 8.15 (1H, d, J= 8.8 Hz), 8.19 (1H, s), 8.34-8.39 (1H, m), 9.13 (1H, s), 12.31 (1H, brs). |
| 30 | 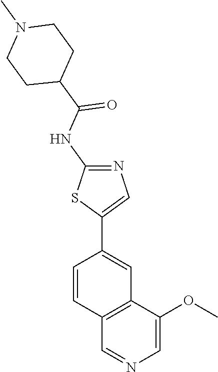 | N-(5-(4- methoxyisoquinolin- 6-yl)thiazol-2-yl)- 1-methylpiperidine- 4-carboxamide | DMSO-d₆; δ 1.59-1.75 (2H, m), 1.78- 1.89 (2H, m), 1.99-2.11 (2H, m), 2.24 (3H, s), 2.54-2.57 (1H, m), 2.84-2.93 (2H, m), 4.06 (3H, s), 8.01-8.05 (1H, m), 8.08-8.19 (4H, m), 8.27 (1H, s), 8.89 (1H, s). |
| 32 | 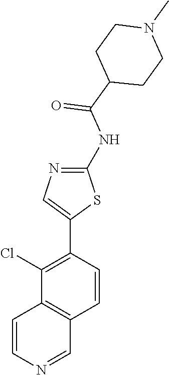 [SMILES: CN1CCC(C(=O)Nc2ncc(-c3ccc4cnccc4c3Cl)s2)CC1] | N-(5-(5- chloroisoquinolin- 6-yl)thiazol-2-yl)- 1-methylpiperidine- 4-carboxamide | DMSO-d₆; δ 1.55-1.65 (1H, m), 1.66- 1.73 (2H, m), 1.80-1.85 (2H, m), 1.86-1.93 (2H, m), 2.17 (3H, s), 2.80- 2.85 (2H, m), 8.05 (1H, d, J = 8.4 Hz), 8.09 (1H, d, J = 6.0 Hz), 8.13 (1H, s), 8.20 (1H, d, J = 8.8 Hz), 8.70 (1H, d, J = 6.0 Hz), 9.40 (1H, s), 12.36 (1H, brs). |
| 35 | 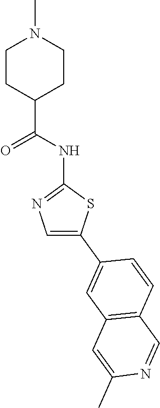 [SMILES: Cc1cc2cc(-c3cnc(NC(=O)C4CCN(C)CC4)s3)ccc2cn1] | 1-methyl-N-(5-(3- methylisoquinolin- 6-yl)thiazol-2- yl)piperidine-4- carboxamide | DMSO-d₆; δ 1.91-2.17 (4H, m), 2.70- 2.80 (6H, m), 2.81-3.06 (4H, m), 3.12-3.52 (1H, m), 8.23 (1H, s), 8.27- 8.34 (2H, m), 8.37-8.43 (1H, m), 8.46 (1H, d, J = 9.6 Hz), 9.71 (1H, s), 10.48-10.76 (1H, m), 12.71 (1H, brs). |
| 38 | 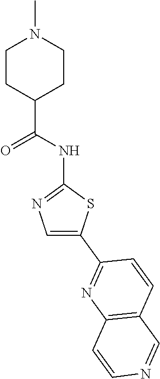 [SMILES: CN1CCC(C(=O)Nc2ncc(-c3ccc4cnccc4n3)s2)CC1] | N-(5-(1,6- naphthyridin-2- yl)thiazol-2-yl)-1- methylpiperidine-4- carboxamide | DMSO-d₆; δ 1.65-1.72 (2H, m), 1.78- 1.84 (2H, m), 1.87-1.94 (2H, m), 2.16 (3H, s), 2.78-2.86 (3H, m), 7.82 (1H, d, J = 6.4 Hz), 8.23 (1H, d, J = 8.4 Hz), 8.47 (1H, s), 8.54 (1H, d, J = 8.8 Hz), 8.68 (1H, d, J = 6.4 Hz), 9.29 (1H, s). |
| 86 | 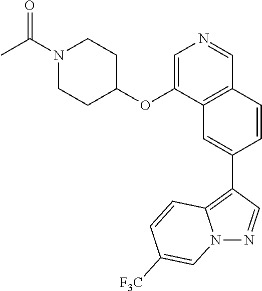 [SMILES: CC(=O)N1CCC(Oc2cncc3ccc(-c4cnn5cc(C(F)(F)F)ccc45)cc23)CC1] | 1-(4-((6-(6- (trifluoromethyl)py- razolo[1,5- a]pyridin-3- yl)isoquinolin-4- yl)oxy)piperidin-1- yl)ethan-1-one | DMSO-d₆; δ 1.72-1.92 (2H, m), 1.98- 2.04 (1H, m), 2.05 (3H, s), 2.07-2.15 (1H, m), 2.59-2.63 (2H, m), 3.73- 3.80 (1H, m), 3.83-3.93 (1H, m), 4.95-5.25 (1H, m), 7.64-7.70 (1H, m), 8.06-8.12 (1H, m), 8.17-8.25 (2H, m), 8.30-8.38 (2H, m), 8.83 (1H, s), 8.96 (1H, s), 9.48 (1H, s). |
| 96 | 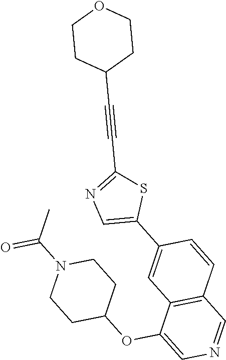 [SMILES: CC(=O)N1CCC(Oc2cncc3ccc(-c4cnc(C#CC5CCOCC5)s4)cc23)CC1] | 1-(4-((6-(2- ((tetrahydro-2H- pyran-4- yl)ethynyl)thiazol- 5-yl)isoquinolin-4- yl)oxy)piperidin-1- yl)ethan-1-one | CD₃OD; δ 1.74-1.83 (2H, m), 1.91- 2.03 (4H, m), 2.12-2.23 (5H, m), 2.97-3.10 (1H, m), 3.55-3.64 (4H, m), 3.86-4.00 (4H, m), 4.98-5.04 (1H, m), 7.97-8.02 (1H, m), 8.14 (1H, d, J = 8.4 Hz), 8.20 (1H, s), 8.30 (1H, s), 8.37 (1H, s), 8.86 (1H, s). |
| 99 | 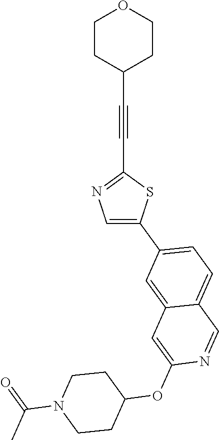 [SMILES: CC(=O)N1CCC(Oc2cc3cc(-c4cnc(C#CC5CCOCC5)s4)ccc3cn2)CC1] | 1-(4-((6-(2- ((tetrahydro-2H- pyran-4- yl)ethynyl)thiazol- 5-yl)isoquinolin-3- yl)oxy)piperidin-1- yl)ethan-1-one | CD₃OD; δ 1.74-2.03 (8H, m), 2.14 (3H, s), 3.00-3.07 (1H, m), 3.54-3.61 (4H, m), 3.80-3.86 (1H, m), 3.91- 3.97 (3H, m), 5.26-5.32 (1H, m), 7.19 (1H, s), 7.72 (1H, dd, J = 8.4, 1.6 Hz), 8.02-8.05 (2H, m), 8.26 (1H, s), 8.96 (1H, s). |

### Examples 21 and 22

### 4-(4-fluorophenoxy)-6-(4-methyl-1H-imidazol-1-yl)isoquinoline (Ex. 21) and 4-(4-fluorophenoxy)-6-(5-methyl-1H-imidazol-1-yl)isoquinoline (Ex. 22)

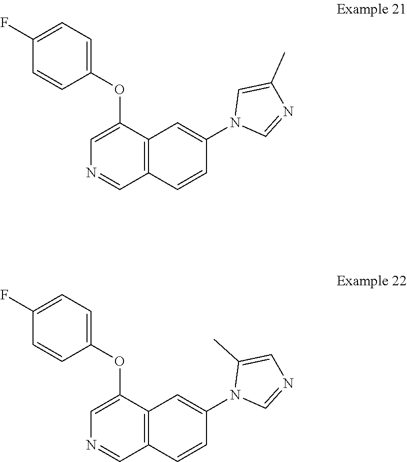 [SMILES: Cc1cn(-c2ccc3cncc(Oc4ccc(F)cc4)c3c2)cn1 ; Cc1cncn1-c1ccc2cncc(Oc3ccc(F)cc3)c2c1]

A mixture of 6-bromo-4-(4-fluorophenoxy)isoquinoline (375 mg, 1.01 mmol), 4-methyl-1H-imidazole (167 mg, 2.03 mmol), CuI (39 mg, 0.20 mmol), K₂CO₃ (281 mg, 2.03 mmol) and D, L-proline (47 mg, 0.41 mmol) in DMSO (5 mL) was stirred at 100° C. for 12 hours under N₂ atmosphere. The reaction mixture was poured into water (70 mL) and extracted with EtOAc (70 mL×2). The combined organic layer was washed with brine (100 mL), dried over anhydrous Na₂SO₄, filtered and concentrated to give the residue. The residue was purified by flash silica gel chromatography (ISCO®; 4 g SepaFlash® Silica Flash Column, Eluent of 3˜4% MeOH/DCM gradient @23 mL/min) and then further purified by SFC separation (column: DAICEL CHIRALCEL OJ (250 mm*30 mm, 10 um); mobile phase: [Neu-EtOH]; B %: 30%-30%) to give Example 21 (26 mg, yield: 8%) as a yellow solid and Example 22 (5.6 mg, yield: 2%) as a yellow solid.

Example 21: ¹H NMR (400 MHz, CD₃OD) δ 2.29 (3H, s), 7.17-7.28 (4H, m), 7.50-7.56 (1H, m), 7.90 (1H, s), 8.02 (1H, dd, J=8.8, 2.0 Hz), 8.27-8.37 (3H, m), 9.04 (1H, s).

Example 22: ¹H NMR (400 MHz, CD₃OD) δ 2.23 (3H, s), 6.93 (1H, s), 7.16-7.23 (4H, m), 7.83 (1H, dd, J=8.8, 2.0 Hz), 7.92 (1H, s), 7.99 (1H, s), 8.21 (1H, d, J=2.0 Hz), 8.39 (1H, d, J=8.8 Hz), 9.14 (1H, s).

### Example 25

### 2-methyl-5-(4-(4-(trifluoromethyl)phenoxy)isoquinolin-6-yl)oxazole

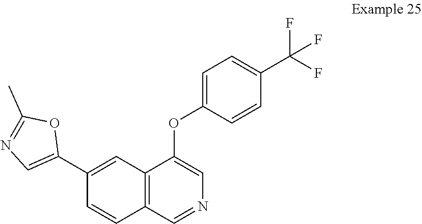 [SMILES: Cc1ncc(-c2ccc3cncc(Oc4ccc(C(F)(F)F)cc4)c3c2)o1]

### Step 1. Synthesis of 6-bromo-4-(4-(trifluoromethyl)phenoxy)isoquinoline

To a solution of compound Int-2 (4.00 g, 12.0 mmol) and 4-(trifluoromethyl)phenol (1.94 g, 12.0 mmol) in DMSO (80 mL) was added CuI (456 mg, 2.40 mmol), K₃PO₄ (5.09 g, 24.0 mmol) and 2,2,6,6-tetramethylheptane-3,5-dione (883 mg, 4.79 mmol) under N₂ atmosphere, the mixture was stirred at 130° C. for 40 hours under N₂ atmosphere. The reaction mixture was diluted with H₂O (300 mL) and extracted with EtOAc (300 mL×2). The combined organic layer was washed with brine (400 mL), dried over anhydrous Na₂SO₄, filtered and concentrated to give the residue. The residue was purified by flash silica gel chromatography (ISCO®; 120 g SepaFlash® Silica Flash Column, Eluent of 0-15% Ethyl acetate/Petroleum ether gradient @75 mL/min) to give 6-bromo-4-(4-(trifluoromethyl)phenoxy)isoquinoline (1.34 g, yield: 17%, purity: 56%) as a yellow gum.

### Step 2. Synthesis of 1-(4-(4-(trifluoromethyl)phenoxy)isoquinolin-6-yl)ethan-1-one

To a solution of 6-bromo-4-(4-(trifluoromethyl)phenoxy)isoquinoline (1.34 g, 2.04 mmol, purity: 56%) and tributyl (1-ethoxyvinyl)tin (1.47 g, 4.08 mmol) in toluene (15 mL) was added Pd(PPh₃)₂Cl₂ (287 mg, 0.408 mmol) under N₂ atmosphere, the mixture was stirred at 100° C. for 16 hours under N₂ atmosphere. The reaction mixture was cooled to 20° C., THF (15 mL) and 2N aqueous HCl (10 mL) were added to the reaction mixture, the mixture was stirred at 20° C. for 3 hours. The reaction mixture was concentrated and the residue was basified with 2N aqueous NaOH to pH=9 and extracted with DCM (50 mL×3). The combined organic layer was dried over anhydrous Na₂SO₄, filtered and concentrated. The residue was purified by flash silica gel chromatography (ISCO®; 20 g SepaFlash® Silica Flash Column, Eluent of 25%˜30% Ethyl acetate/Petroleum ether gradient @25 mL/min) to give 1-(4-(4-(trifluoromethyl)phenoxy)isoquinolin-6-yl)ethan-1-one (460 mg, yield: 62%) as a yellow solid.

¹H NMR (400 MHz, CDCl₃) δ 2.73 (3H, s), 7.20 (2H, d, J=8.4 Hz), 7.68 (2H, d, J=8.4 Hz), 8.11-8.17 (1H, m), 8.21-8.29 (2H, m), 8.70 (1H, s), 9.18 (1H, s).

### Step 3. Synthesis of 2-methyl-5-(4-(4-(trifluoromethyl)phenoxy)isoquinolin-6-yl)oxazole

1-(4-(4-(trifluoromethyl)phenoxy)isoquinolin-6-yl)ethan-1-one (200 mg, 0.603 mmol) was added to a solution of iodosobenzene (299 mg, 1.36 mmol) and TfOH (408 mg, 2.72 mmol) in MeCN (8 mL) at 0° C. and the reaction mixture was stirred at 0° C. for 6 minutes and stirred at 80° C. for 20 hours. The reaction mixture was quenched with saturated aqueous Na₂SO₃ (20 mL), basified with saturated aqueous NaHCO₃ to pH=8 and extracted with EtOAc (40 mL×3). The combined organic layer was washed with brine (60 mL), dried over anhydrous Na₂SO₄, filtered and concentrated. The residue was purified by prep-HPLC (0.05% NH₃H₂O+10 mM NH₄HCO₃ as an additive) and lyophilized to give the title compound (56 mg, yield: 24%) as a light yellow solid. ¹H NMR (400 MHz, CDCl₃) δ 2.57 (3H, s), 7.14-7.21 (2H, m), 7.43 (1H, s) 7.62-7.68 (2H, m), 7.89 (1H, dd, J=8.4, 1.6 Hz), 8.08 (1H, d, J=8.4 Hz), 8.22 (1H, s), 8.26 (1H, s), 9.09 (1H, s).

### Example 26

### 4-ethyl-N-(5-(isoquinolin-6-yl)thiazol-2-yl)piperazine-1-carboxamide

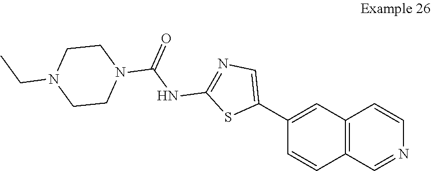 [SMILES: CCN1CCN(C(=O)Nc2ncc(-c3ccc4cnccc4c3)s2)CC1]

To a solution of compound Int-4 (100 mg, 0.440 mmol) in DMF (3 mL) was added CDI (107 mg, 0.660 mmol) at 25° C., then the mixture was stirred at 25° C. for 16 hours. 1-ethylpiperazine (75 mg, 0.66 mmol) was added to the reaction mixture at 25° C. The resulting reaction mixture was stirred at 25° C. for another 16 hours. The reaction mixture was concentrated and the residue was purified by prep-HPLC (0.225% FA as an additive; Method C) to give the title compound (9 mg, yield: 4.9%, FA salt) as a yellow solid.

¹H NMR (400 MHz, DMSO-d₆) δ 1.03 (3H, t, J=7.2 Hz), 2.30-2.38 (2H, m), 2.39-2.42 (4H, m), 3.52-3.60 (4H, m), 7.83 (1H, d, J=5.6 Hz), 8.01 (1H, dd, J=8.4, 1.6 Hz), 8.03-8.08 (2H, m), 8.12 (1H, d, J=8.4 Hz), 8.16 (1H, s), 8.49 (1H, d, J=5.6 Hz), 9.26 (1H, brs).

### Example 27

### 5-(3-(isoquinolin-6-yl)pyrazolo[1,5-a]pyridin-6-yl)-1-methylpyridin-2(1H)-one

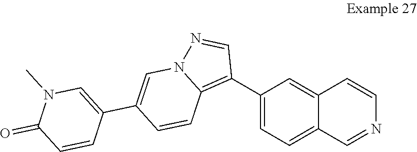 [SMILES: Cn1cc(-c2ccc3c(-c4ccc5cnccc5c4)cnn3c2)ccc1=O]

### Step 1. Synthesis of 6-(6-bromopyrazolo[1,5-a]pyridin-3-yl)isoquinoline

A mixture of 6-bromo-3-iodopyrazolo[1,5-a]pyridine (160 mg, 0.495 mmol), compound Int-1 (126 mg, 0.495 mmol), Pd(dppf)Cl₂ (54.4 mg, 0.074 mmol) and Na₂CO₃ (158 mg, 1.47 mmol) in dioxane (3 mL) and H₂O (0.3 mL) was degassed and purged with N₂ for 3 times. Then the mixture was stirred at 90° C. for 3 hours under N₂ atmosphere. The reaction mixture was turned into black suspension from brown suspension. The reaction mixture was diluted with water (30 mL) and EtOAc (30 ml), filtered through a pad of celite. The filtrate was extracted with EtOAc (30 mL×2) and the combined organic layer was washed with brine (30 mL), dried over anhydrous Na₂SO₄, filtered and concentrated. The residue was purified by flash column (SiO₂, 75% EtOAc in PE) to give 6-(6-bromopyrazolo[1,5-a]pyridin-3-yl)isoquinoline (138 mg, yield: 86%) as a yellow solid.

¹H NMR (400 MHz, DMSO-d₆) δ 7.61 (1H, d, J=9.6 Hz), 7.94 (1H, d, J=5.6 Hz), 8.13 (1H, d, J=8.4 Hz), 8.24-8.28 (2H, m), 8.34 (1H, s), 8.56 (1H, d, J=5.6 Hz), 8.70 (1H, s), 9.27 (1H, s), 9.35 (1H, s).

### Step 2. Synthesis of 5-(3-(isoquinolin-6-yl)pyrazolo[1,5-a]pyridin-6-yl)-1-methylpyridin-2(1H)-one

A mixture of 6-(6-bromopyrazolo[1,5-a]pyridin-3-yl)isoquinoline (100 mg, 0.308 mmol), 1-methyl-5-(4,4,5,5-tetramethyl-1,3,2-dioxaborolan-2-yl)pyridin-2(1H)-one (80 mg, 0.34 mmol), Pd(dppf)Cl₂ (34 mg, 0.049 mmol) and Na₂CO₃ (98 mg, 0.93 mmol) in dioxane (5 mL) and H₂O (0.5 mL) was degassed and purged with N₂ for 3 times, and then the mixture was stirred at 90° C. for 3 hours under N₂ atmosphere. The reaction mixture was turned into black suspension from brown suspension. The residue was diluted saturated H₂O (30 mL) and extracted with DCM/MeOH (30 mL×3, 10/1). The combined organic layer was washed with brine (30 mL), dried over anhydrous Na₂SO₄ and concentrated. The residue was purified by prep-HPLC (0.025% FA as an additive), then lyophilized to give the title compound (40 mg, yield: 37%) as a yellow solid. 1H NMR (400 MHz, DMSO-d₆) δ 3.55 (3H, s), 6.54 (1H, d, J=9.6 Hz), 7.73 (1H, dd, J=9.6, 1.6 Hz), 7.90 (1H, d, J=5.6 Hz), 8.00 (1H, dd, J=9.6, 2.8 Hz), 8.11 (1H, dd, J=8.4, 1.6 Hz), 8.14 (1H, s), 8.17-8.23 (1H, m), 8.27-8.36 (3H, m), 8.50 (1H, d, J=5.6 Hz), 8.65 (1H, s), 9.10 (1H, s), 9.29 (1H, s).

The following compounds were synthesized analogously to Example 27

| Example | | | |
|---|---|---|---|
| No. | Structure | Name | ¹H NMR (400 MHz) |
| 43 | 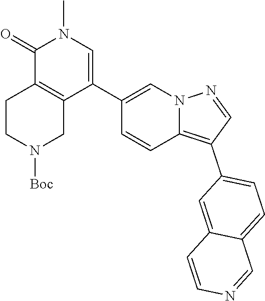 [SMILES: Cn1cc(-c2ccc3c(-c4ccc5cnccc5c4)cnn3c2)c2c(c1=O)CCN(C(=O)OC(C)(C)C)C2] | tert-butyl-8-(3- (isoquinolin-6- yl)pyrazolo[1,5- a]pyridin-6-yl)-6-methyl- 5-oxo-3,4,5,6-tetrahydro- 2,6-naphthyridine-2(1H)- carboxylate | CDCl₃; δ 1.87 (9H, s), 2.76 (2H, t, J = 5.6 Hz), 3.62 (3H, s), 3.644 (2H, t, J = 5.6 Hz), 4.29 (2H, s), 7.19-7.25 (2H, m), 7.75 (1H, d, J = 5.6 Hz), 7.91-7.99 (2H, m), 8.04 (1H, s), 8.11 (1H, d, J = 8.8 Hz), 8.35 (1H, s), 8.46 (1H, s), 8.55 (1H, d, J = 5.6 Hz), 9.29 (1H, s). |
| 46 | 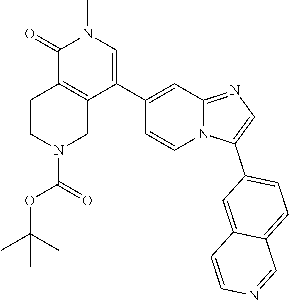 [SMILES: Cn1cc(-c2ccn3c(-c4ccc5cnccc5c4)cnc3c2)c2c(c1=O)CCN(C(=O)OC(C)(C)C)C2] | tert-butyl-8-(3- (isoquinolin-6- yl)imidazo[1,2-a]pyridin- 7-yl)-6-methyl-5-oxo- 3,4,5,6-tetrahydro-2,6- naphthyridine-2(1H)- carboxylate | CDCl₃; δ 1.45 (9H, s), 2.76 (2H, t, J = 6.0 Hz), 3.63 (3H, s), 3.66 (2H, t, J = 6.0 Hz), 4.33 (2H, s), 6.88 (1H, d, J = 6.0 Hz), 7.22 (1H, s), 7.65 (1H, s), 7.76 (1H, d, J = 5.6 Hz), 7.86 (1H, dd, J = 8.4, 1.6 Hz), 7.93 (1H, s), 8.07 (1H, s), 8.17 (1H, d, J = 8.8 Hz), 8.49 (1H, d, J = 7.2 Hz), 8.63 (1H, d, J = 5.6 Hz), 9.35 (1H, s). |
| 51 | 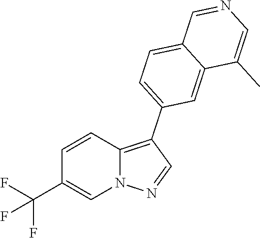 [SMILES: Cc1cncc2ccc(-c3cnn4cc(C(F)(F)F)ccc34)cc12] | 4-methyl-6-(6- (trifluoromethyl)pyrazolo [1,5-a]pyridin-3- yl)isoquinoline | DMSO-d₆; δ 2.68 (3H, s) 7.62 (1H, dd, J = 9.6, 1.6 Hz), 8.09 (1H, dd, J = 8.8, 1.6 Hz), 8.23 (1H, d, J = 8.4 Hz), 8.25 (1H, s), 8.35 (1H, d, J = 9.2 Hz), 8.38 (1H, s), 8.87 (1H, s), 9.17 (1H, s), 9.48 (1H, s). |
| 54 | 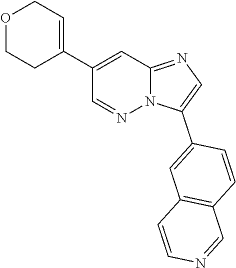 [SMILES: C1=C(c2cnn3c(-c4ccc5cnccc5c4)cnc3c2)CCOC1] | 6-(7-(3,6-dihydro-2H- pyran-4-yl)imidazo[1,2- b]pyridazin-3- yl)isoquinoline | DMSO-d₆; δ 2.55-2.60 (2H, m), 3.88 (2H, t, J = 5.6 Hz), 4.30-4.33 (2H, m), 6.77-6.79 (1H, m), 7.86 (1H, d, J = 5.6 Hz), 8.14 (1H, d, J = 2.0 Hz), 8.23 (1H, d, J = 8.8 Hz), 8.38 (1H, dd, J = 8.4, 1.2 Hz), 8.51 (1H, s), 8.53 (1H, d, J = 5.6 Hz), 8.89 (1H, s), 9.08 (1H, d, J = 2.0 Hz), 9.31 (1H, s). |
| 93 | 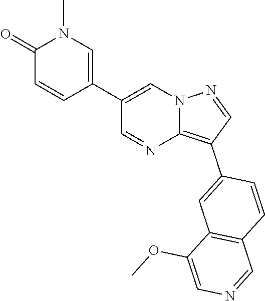 [SMILES: COc1cncc2ccc(-c3cnn4cc(-c5ccc(=O)n(C)c5)cnc34)cc12] | 5-(3-(4- methoxyisoquinolin-6- yl)pyrazolo[1,5- a]pyrimidin-6-yl)-1- methylpyridin-2(1H)-one | DMSO-d₆; δ 3.55 (3H, s), 4.10 (3H, s), 6.57 (1H, d, J = 9.2 Hz), 8.00-8.06 (1H, m), 8.12-8.21 (2H, m), 8.40-8.46 (2H, m), 8.91 (1H, s), 8.96- 9.01 (2H, m), 9.10 (1H, d, J = 2.0 Hz), 9.47 (1H, d, J = 2.0 Hz) |

### Example 31

### 1-methyl-N-(5-(1-oxo-2,6-naphthyridin-2(1H)-yl)thiazol-2-yl)piperidine-4-carboxamide

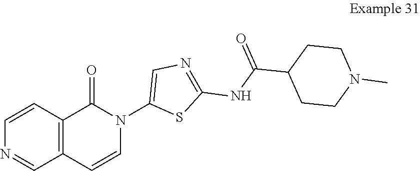 [SMILES: CN1CCC(C(=O)Nc2ncc(-n3ccc4cnccc4c3=O)s2)CC1]

A solution of 2,6-naphthyridin-1(2H)-one (200 mg, 1.37 mmol), N-(5-bromothiazol-2-yl)-1-methylpiperidine-4-carboxamide (541 mg, 1.78 mmol), CuI (52 mg, 0.27 mmol) and K₃PO₄ (581 mg, 2.74 mmol) in DMSO (6 mL) was bubbled with N₂ for 6 minutes. DMEA (48 mg, 0.55 mmol) was added to the reaction mixture, the mixture was stirred at 100° C. for 12 hours under N₂ atmosphere. The reaction mixture was diluted with H₂O (100 mL), extracted with EtOAc (100 mL×3). The combined organic layer was dried over anhydrous Na₂SO₄, filtered and concentrated. The residue was purified by flash silica gel chromatography (ISCO®; 4 g SepaFlash® Silica Flash Column, Eluent of 20˜30% MeOH/DCM gradient @25 mL/min), then further purified by prep-HPLC (0.225% FA as an additive; Method C) and lyophilized to give the title compound (8 mg, yield: 1%, FA salt) as a yellow solid. ¹H NMR (400 MHz, DMSO-d₆) δ 1.62-1.71 (2H, m), 1.76-1.81 (2H, m), 1.85-1.91 (2H, m), 2.17 (3H, s), 2.41-2.44 (1H, m), 2.79-2.84 (2H, m), 6.98 (1H, d, J=7.2 Hz), 7.86 (1H, s), 7.99 (1H, d, J=7.6 Hz), 8.10 (1H, d, J=5.2 Hz), 8.23 (1H, s), 8.73 (1H, d, J=5.6 Hz), 9.16 (1H, s), 12.15 (1H, brs).

### Example 33

### 1-methyl-N-(5-(5-methylisoquinolin-6-yl)thiazol-2-yl)piperidine-4-carboxamide

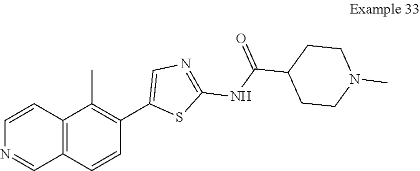 [SMILES: Cc1c(-c2cnc(NC(=O)C3CCN(C)CC3)s2)ccc2cnccc12]

A mixture of compound Int-11 (100 mg, 0.444 mmol), 1-methyl-N-(thiazol-2-yl)piperidine-4-carboxamide (108 mg, 0.488 mmol), Pd(OAc)₂ (10 mg, 0.044 mmol), t-Bu₃PHBF₄ (26 mg, 0.089 mmol) and Cs₂CO₃ (289 mg, 0.888 mmol) in anhydrous DMF (3 mL) was degassed and purged with N₂ for 3 times. Then the mixture was stirred at 120° C. for 2 hours under N₂ atmosphere. The reaction mixture was quenched with H₂O (25 mL) and extracted with EtOAc (60 mL×3). The combined organic layer was washed with brine (25 mL), dried over anhydrous Na₂SO₄, filtered and concentrated. The crude product was triturated with CH₃CN (2 mL), then purified by prep-HPLC (0.225% FA as an additive; Method C) and lyophilized to give the title compound (24.14 mg, yield: 13%, FA salt) as a white solid. ¹H NMR (400 MHz, CD₃OD) δ 1.99-2.10 (2H, m), 2.10-2.20 (2H, m), 2.73-2.81 (7H, m), 2.85-2.90 (2H, m), 3.40-3.45 (2H, m), 7.54 (1H, s), 7.69 (1H, d, J=8.4 Hz), 8.02 (1H, d, J=8.4 Hz), 8.07 (1H, d, J=6.0 Hz), 8.47 (1H, s), 8.54 (1H, d, J=6.4 Hz), 9.24 (1H, s).

The following compounds were synthesized analogously to Example 33

| Example | | | |
|---|---|---|---|
| No. | Structure | Name | ¹H NMR (400 MHz) |
| 36 | 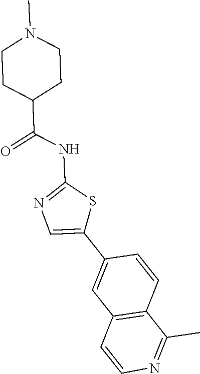 [SMILES: Cc1nccc2cc(-c3cnc(NC(=O)C4CCN(C)CC4)s3)ccc12] | 1-methyl-N-(5-(1- methylisoquinolin- 6-yl)thiazol-2- yl)piperidine-4- carboxamide | DMSO-d₆; δ 1.64-1.77 (2H, m), 1.80- 1.90 (2H, m), 2.00-2.05 (2H, m), 2.25 (3H, s), 2.50-2.55 (1H, m), 2.88 (3H, s), 2.95-3.00 (2H, m), 7.68 (1H, d, J = 6.0 Hz), 7.99 (1H, dd, J = 8.8, 2.0 Hz), 8.11 (1H, d, J = 1.6 Hz), 8.14 (1H, s), 8.20 (1H, s), 8.22 (1H, d, J = 8.8 Hz), 8.34 (1H, d, J = 5.6 Hz), 12.35 (1H, brs). |
| 37 | 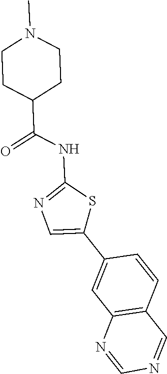 [SMILES: CN1CCC(C(=O)Nc2ncc(-c3ccc4cncnc4c3)s2)CC1] | 1-methyl-N-(5- (quinazolin-7- yl)thiazol-2- yl)piperidine-4- carboxamide | DMSO-d₆; δ 1.80-1.95 (2H, m), 2.05- 2.10 (2H, m), 2.70-2.80 (4H, m), 2.95- 3.05 (2H, m), 3.40-3.50 (2H, m), 7.94- 8.03 (2H, m), 8.05-8.15 (2H, m), 8.28 (1H, s), 9.14 (1H, s), 9.43 (1H, s). |
| 39 | 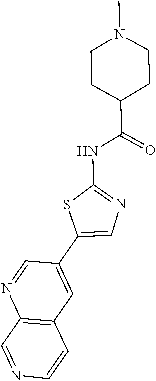 [SMILES: CN1CCC(C(=O)Nc2ncc(-c3cnc4cnccc4c3)s2)CC1] | N-(5-(1,7- naphthyridin-3- yl)thiazol-2-yl)-1- methylpiperidine-4- carboxamide | CD₃OD; δ 1.96-2.13 (2H, m), 2.21-2.34 (2H, m), 2.90-3.00 (4H, m), 3.10-3.15 (2H, m), 3.60-3.70 (2H, m), 8.06 (1H, d, J = 6.0 Hz), 8.23 (1H, s), 8.58 (1H, s), 8.62 (1H, d, J = 5.6 Hz), 9.45-9.55 (2H, m). |
| 40 | 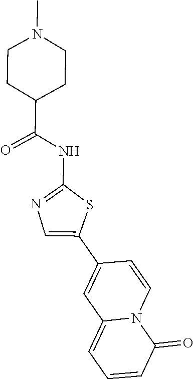 [SMILES: CN1CCC(C(=O)Nc2ncc(-c3ccn4c(=O)cccc4c3)s2)CC1] | 1-methyl-N-(5-(4- oxo-4H-quinolizin- 8-yl)thiazol-2- yl)piperidine-4- carboxamide | DMSO-d₆; δ 1.61-1.74 (2H, m), 1.77- 1.86 (2H, m), 1.88-1.97 (2H, m), 2.19 (3H, s), 2.53-2.56 (1H, m), 2.79-2.89 (2H, m), 6.37 (1H, d, J = 7.6 Hz), 6.85 (1H, d, J = 8.0 Hz), 7.56 (1H, d, J = 7.6 Hz), 7.68-7.76 (1H, m), 7.90 (1H, d, J = 2.0 Hz), 8.25 (1H, s), 8.90 (1H, d, J = 8.0 Hz). |
| 87 | 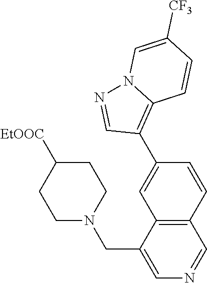 [SMILES: CCOC(=O)C1CCN(Cc2cncc3ccc(-c4cnn5cc(C(F)(F)F)ccc45)cc23)CC1] | ethyl 1-((6-(6- (trifluoromethyl)py- razolo[1,5- a]pyridin-3- yl)isoquinolin-4- yl)methyl)piperidine- 4-carboxylate | — |
| 90 | 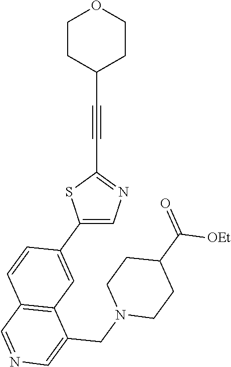 [SMILES: CCOC(=O)C1CCN(Cc2cncc3ccc(-c4cnc(C#CC5CCOCC5)s4)cc23)CC1] | ethyl 1-((6-(2- ((tetrahydro-2H- pyran-4- yl)ethynyl)thiazol- 5-yl)isoquinolin-4- yl)methyl)piperidine- 4-carboxylate | — |
| 98 | 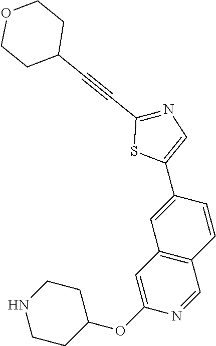 [SMILES: C(#CC1CCOCC1)c1ncc(-c2ccc3cnc(OC4CCNCC4)cc3c2)s1] | 5-(3-(piperidin-4- yloxy)isoquinolin- 6-yl)-2- ((tetrahydro-2H- pyran-4- yl)ethynyl)thiazole | CD₃OD; δ 1.75-1.87 (2H, m), 1.95-2.05 (2H, m), 2.12-2.20 (2H, m), 2.25-2.36 (2H, m), 2.98-3.13 (1H, m), 3.27-3.32 (2H, m), 3.45-3.53 (2H, m), 3.56-3.66 (2H, m), 3.89-4.01 (2H, m), 5.38-5.45 (1H, m), 7.26 (1H, s), 7.77 (1H, dd, J = 8.4, 1.6 Hz), 8.03-8.11 (2H, m), 8.30 (1H, s), 9.01 (1H, s). |

### Example 42

### 5-(isoquinolin-6-yl)-2-((tetrahydro-2H-pyran-4-yl)ethynyl)thiazole

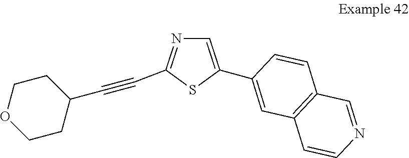 [SMILES: C(#CC1CCOCC1)c1ncc(-c2ccc3cnccc3c2)s1]

### Step 1. Synthesis of 2-bromo-5-(isoquinolin-6-yl)thiazole

A mixture of CuBr₂ (590 mg, 2.64 mmol) and isoamyl nitrite (464 mg, 3.96 mmol) in DMF (4 mL) was added compound Int-4 (300 mg, 1.32 mmol) in DMF (4 mL) at 0° C. The reaction mixture was stirred at 0° C. for 0.5 hour and stirred at 50° C. for 3 hours. The reaction mixture was poured into saturated aqueous NaHCO₃ (30 mL) and filtered, the solid was suspended in a solution of DCM/MeOH (30 mL, 10/1) and filtered. The filtrate was concentrated and the residue was purified by silica gel column (PE/EtOAc=2/1) to give 2-bromo-5-(isoquinolin-6-yl)thiazole (90 mg, yield: 23%) as a white solid. ¹H NMR (400 MHz, DMSO-d₆) δ 7.86 (1H, d, J=6.0 Hz), 8.02 (1H, dd, J=8.8, 2.0 Hz), 8.18-8.27 (2H, m), 8.36 (1H, s), 8.55 (1H, d, J=6.0 Hz), 9.33 (1H, s).

### Step 2. Synthesis of 5-(isoquinolin-6-yl)-2-((tetrahydro-2H-pyran-4-yl)ethynyl)thiazole

A mixture of 2-bromo-5-(isoquinolin-6-yl)thiazole (60 mg, 0.21 mmol), 4-ethynyltetrahydro-2H-pyran (68 mg, 0.62 mmol), CuI (8 mg, 0.04 mmol), Pd(PPh₃)₂Cl₂ (29 mg, 0.041 mmol) and Et₃N (104 mg, 1.03 mmol) in THF (4 mL) was degassed and purged with N₂ for 3 times at 0° C., and then the mixture was stirred at 65° C. for 16 hours under N₂ atmosphere. The reaction mixture was concentrated and the residue was purified by flash silica gel chromatography (PE/EtOAc=2/1), then further purified by prep-HPLC (0.04% NH₃H₂O+10 mM NH₄HCO₃ as an additive) and lyophilized to afford the title compound (9.73 mg, yield: 14%) as a yellow solid. ¹H NMR (400 MHz, CDCl₃) δ 1.81-1.92 (2H, m), 1.95-2.03 (2H, m), 2.93-3.03 (1H, m), 3.56-3.64 (2H, m), 3.96-4.03 (2H, m), 7.72 (1H, d, J=5.6 Hz), 7.84 (1H, dd, J=8.8, 1.6 Hz), 7.99 (1H, s), 8.06 (1H, d, J=8.4 Hz), 8.15 (1H, s), 8.59 (1H, d, J=5.6 Hz), 9.29 (1H, s).

### Example 44

### 4-(3-(isoquinolin-6-yl)pyrazolo[1,5-a]pyridin-6-yl)-2-methyl-5,6,7,8-tetrahydro-2,6-naphthyridin-1(2H)-one

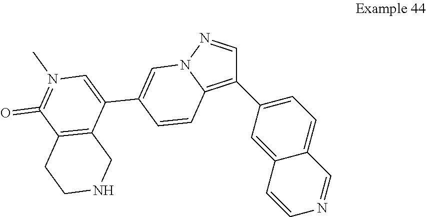

To a solution of Example 43 (60 mg, 0.12 mmol) in DCM (1 mL) was added TFA (0.5 mL). The mixture was stirred at 25° C. for 3 hours. The reaction mixture was concentrated and the residue was purified by prep-PLC (0.225% TFA as an additive; Method C), then lyophilized to afford the title compound (37.23 mg, yield: 60%, TFA salt) as a yellow solid. ¹H NMR (400 MHz, CD₃OD-d₄) a 2.95 (2H, t, J=6.4 Hz), 3.56 (2H, t, J=6.4 Hz), 3.69 (3H, s), 4.23 (2H, s), 7.56 (1H, dd, J=9.2, 1.6 Hz), 7.82 (1H, s), 8.37-8.44 (2H, m), 8.45-8.49 (1H, m), 8.53 (1H, s), 8.54-8.56 (1H, m), 8.59 (1H, s), 8.74 (1H, s), 8.80 (1H, s), 9.63 (1H, s).

The following compounds were synthesized analogously to Example 44

| Example | | | |
|---|---|---|---|
| No. | Structure | Name | ¹H NMR (400 MHz) |
| 47 | 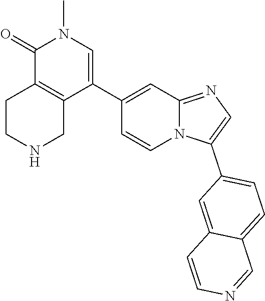 [SMILES: Cn1cc(-c2ccn3c(-c4ccc5cnccc5c4)cnc3c2)c2c(c1=O)CCNC2] | 4-(3-(isoquinolin-6- yl)imidazo[1,2- a]pyridin-7-yl)-2- methyl-5,6,7,8- tetrahydro-2,6- naphthyridin-1(2H)- one | DMSO-d₆; δ 2.74 (2H, t, J = 5.6 Hz), 3.35-3.45 (2H, m), 3.56 (3H, s), 4.17 (2H, s), 7.34 (1H, d, J = 6.8 Hz), 7.90-7.95 (2H, m), 8.17-8.30 (2H, m), 8.46-8.59 (3H, m), 8.69 (1H, d, J = 6.0 Hz), 9.09 (1H, d, J = 6.8 Hz), 9.30-9.40 (2H, m), 9.67 (1H, s). |
| 50 | 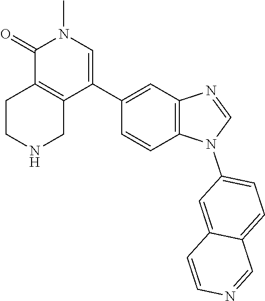 [SMILES: Cn1cc(-c2ccc3c(c2)ncn3-c2ccc3cnccc3c2)c2c(c1=O)CCNC2] | 4-(1-(isoquinolin-6- yl)-1H- benzo[d]imidazol-5- yl)-2-methyl-5,6,7,8- tetrahydro-2,6- naphthyridin-1(2H)- one | DMSO-d₆; δ 3.00-3.10 (3H, m), 3.45-3.50 (4H, m), 3.65-3.75 (2H, m), 7.32 (1H, d, J = 8.0 Hz), 7.56 (1H, s), 7.74 (1H, s), 7.86 (1H, d, J = 8.4 Hz), 7.98 (1H, d, J = 6.4 Hz), 8.07 (1H, dd, J = 8.8, 2.0 Hz), 8.36 (1H, s), 8.42 (1H, d, J = 8.8 Hz), 8.61 (1H, d, J = 5.6 Hz), 8.82 (1H, s), 9.45 (1H, s). |

### Example 45

### 4-(3-(isoquinolin-6-yl)pyrazolo[1,5-a]pyridin-6-yl)-2,6-dimethyl-5,6,7,8-tetrahydro-2,6-naphthyridin-1(2H)-one

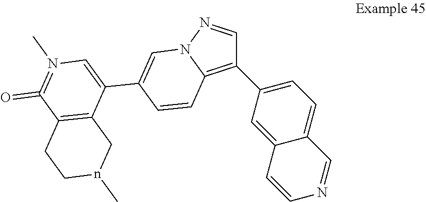

To a solution of Example 44 (120 mg, 0.230 mmol, TFA salt) in CH₃OH (3 mL) was added DIPEA (30 mg, 0.23 mmol). The mixture was stirred at 25° C. for 0.5 hour. Then HOAc (14 mg, 0.23 mmol) was added to adjust the pH=5 and 37% aqueous HCHO (93 mg, 1.2 mmol) was added and the mixture was stirred at 25° C. for 0.5 hour. Then the NaBH₃CN (43 mg, 0.69 mmol) was added and the mixture was stirred at 25° C. for another 1 hour. The reaction mixture was concentrated and the residue was purified by prep-HPLC (0.225% FA as an additive; Method C), then lyophilized to afford the title compound (11.57 mg, yield: 11%, FA salt) as a yellow solid. ¹H NMR (400 MHz, DMSO-d₆) δ 2.26 (3H, s), 2.53-2.59 (4H, m), 3.24-3.25 (2H, m), 3.48 (3H, s), 7.40-7.49 (1H, m), 7.70 (1H, s), 7.90 (1H, d, J=5.6 Hz), 8.09-8.14 (1H, m), 8.17-8.21 (1H, m), 8.24 (1H, s), 8.26 (1H, d, J=9.2 Hz), 8.33 (1H, s), 8.50 (1H, d, J=6.0 Hz), 8.67 (1H, s), 8.80 (1H, s), 9.28 (1H, s).

The following compounds were synthesized analogously to Example 45

| Example | | | |
|---|---|---|---|
| No. | Structure | Name | ¹H NMR (400 MHz) |
| 48 | 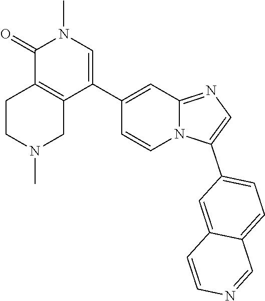 [SMILES: CN1CCc2c(c(-c3ccn4c(-c5ccc6cnccc6c5)cnc4c3)cn(C)c2=O)C1] | 4-(3-(isoquinolin-6- yl)imidazo[1,2- a]pyridin-7-yl)-2,6- dimethyl-5,6,7,8- tetrahydro-2,6- naphthyridin-1(2H)- one | DMSO-d₆; δ 2.27 (3H, s), 2.50-2.55 (2H, m), 2.58-2.60 (2H, m), 3.28 (2H, s), 3.50 (3H, s), 7.05 (1H, dd, J = 7.2, 1.6 Hz), 7.65 (1H, s), 7.69 (1H, s), 7.93 (1H, d, J = 6.0 Hz), 8.03 (1H, dd, J = 8.4, 1.6 Hz), 8.08 (1H, s), 8.27 (1H, s), 8.29 (1H, d, J = 3.6 Hz), 8.35 (1H, s), 8.56 (1H, d, J = 5.6 Hz), 8.85 (1H, d, J = 6.4 Hz), 9.36 (1H, s). |
| 76 | 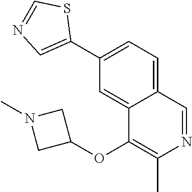 [SMILES: Cc1ncc2ccc(-c3cncs3)cc2c1OC1CN(C)C1] | 5-(3-methyl-4-((1- methylazetidin-3- yl)oxy)isoquinolin-6- yl)thiazole | DMSO-d₆; δ 2.55 (3H, s), 2.56 (3H, s), 3.64-3.74 (2H, m), 3.92-4.00 (2H, m), 4.66-4.75 (1H, m), 8.01 (1H, dd, J = 8.4, 2.8 Hz), 8.11 (1H, s), 8.20 (1H, d, J = 8.8 Hz), 8.60 (1H, s), 9.06 (1H, s), 9.23 (1H, s) |
| 78 | 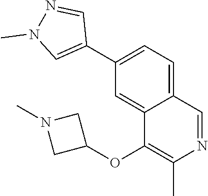 [SMILES: Cc1ncc2ccc(-c3cnn(C)c3)cc2c1OC1CN(C)C1] | 3-methyl-6-(1- methyl-1H-pyrazol-4- yl)-4-((1- methylazetidin-3- yl)oxy)isoquinoline | DMSO-d₆, δ 2.37 (3H, s), 2.53 (3H, s), 3.34-3.42 (2H, m), 3.72-3.75 (2H, m), 3.92 (3H, s), 4.56-4.62 (1H, m), 7.80-7.94 (1H, m), 7.98 (1H, s), 8.07 (1H, d, J = 2.4 Hz), 8.13-8.20 (1H, m), 8.39 (1H, s), 8.94 (1H, s). |
| 97 | 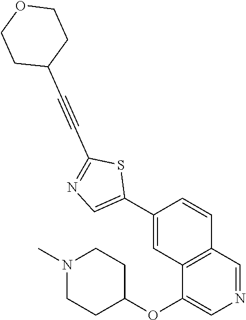 [SMILES: CN1CCC(Oc2cncc3ccc(-c4cnc(C#CC5CCOCC5)s4)cc23)CC1] | 5-(4-((1- methylpiperidin-4- yl)oxy)isoquinolin-6- yl)-2-((tetrahydro- 2H-pyran-4- yl)ethynyl)thiazole | DMSO-d₆; δ 1.63-1.71 (2H, m), 1.87-1.95 (4H, m), 2.03-2.14 (2H, m), 2.34 (3H, s), 2.43-2.49 (2H, m), 2.75- 2.82 (2H, m), 3.05-3.08 (1H, m), 3.78-3.86 (4H, m), 4.80-4.83 (1H, m), 8.06-8.11 (1H, m), 8.13- 8.21 (1H, m), 8.27 (1H, s), 8.31 (1H, s), 8.52 (1H, s), 8.95 (1H, s). |
| 100 | 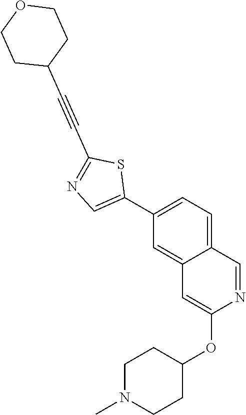 [SMILES: CN1CCC(Oc2cc3cc(-c4cnc(C#CC5CCOCC5)s4)ccc3cn2)CC1] | 5-(3-((1- methylpiperidin-4- yl)oxy)isoquinolin-6- yl)-2-((tetrahydro- 2H-pyran-4- yl)ethynyl)thiazole | CD₃OD; δ 1.74-1.89 (2H, m), 1.92- 2.04 (2H, m), 2.05-2.17 (2H, m), 2.18-2.31 (2H, m), 2.74 (3H, s), 2.95-3.14 (3H, m), 3.20-3.29 (2H, m), 3.55-3.65 (2H, m), 3.90-4.01 (2H, m), 5.22-5.36 (1H, m), 7.22 (1H, s), 7.75 (1H, dd, J = 8.4, 1.6 Hz), 8.00-8.10 (2H, m), 8.29 (1H, s), 8.99 (1H, s). |
| 107 | 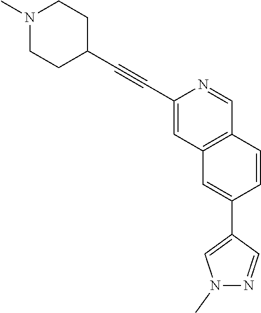 [SMILES: CN1CCC(C#Cc2cc3cc(-c4cnn(C)c4)ccc3cn2)CC1] | 6-(1-methyl-1H- pyrazol-4-y1)-3-((1- methylpiperidin-4- yl)ethynyl)isoquinoline | DMSO-d₆; δ 1.65-1.76 (2H, m), 1.90-1.98 (2H, m), 2.28 (3H, s), 2.52-2.54 (1H, m), 2.69-2.90 (4H, m), 3.91 (3H, s), 7.85 (1H, s), 7.92 (1H, dd, J = 8.4, 1.6 Hz), 8.05 (1H, s), 8.07-8.12 (2H, m), 8.35 (1H, s), 9.16 (1H, s). |

### Example 49

### tert-butyl 8-(1-isoquinolin-6-yl)-1H-benzo[d]imidazol-5-yl)-6-methyl-5-oxo-3,4,5,6-tetrahydro-2,6-naphthyridine-2(1H)-carboxylate

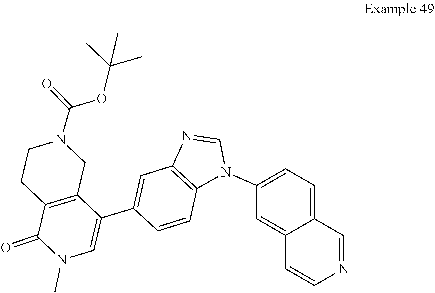 [SMILES: Cn1cc(-c2ccc3c(c2)ncn3-c2ccc3cnccc3c2)c2c(c1=O)CCN(C(=O)OC(C)(C)C)C2]

### Step 1. Synthesis of N-(4-bromo-2-nitrophenyl)isoquinolin-6-amine

To a solution of isoquinolin-6-amine (1.00 g, 6.94 mmol) in DMF (20 mL) was added NaH (417 mg, 10.4 mmol, 60% dispersion in mineral oil) at 0° C. and stirred for 0.5 hour. 4-bromo-1-fluoro-2-nitrobenzene (1.53 g, 6.94 mmol) was added to the reaction mixture at 0° C. and the resulting mixture was stirred at 0° C. for another 0.5 hour. The reaction mixture was quenched by addition H₂O (50 mL) and extracted with EtOAc (50 mL×2). The combined organic layers were washed with water (20 mL×2), brine (20 mL), dried over anhydrous Na₂SO₄, filtered and concentrated. The residue was purified by Combi Flash (0% to 60% EtOAc in PE) to give N-(4-bromo-2-nitrophenyl)isoquinolin-6-amine (500 mg, yield: 21%) as a yellow solid.

### Step 2. Synthesis of 4-bromo-N¹-(isoquinolin-6-yl)benzene-1,2-diamine

A mixture of N-(4-bromo-2-nitrophenyl)isoquinolin-6-amine (200 mg, 0.581 mmol), Fe powder (130 mg, 2.32 mmol) and NH₄Cl (125 mg, 2.32 mmol) in EtOH (2 mL) and H₂O (2 mL) was stirred at 75° C. for 2 hours. The reaction mixture was filtered through a pad of celite and the solid was washed with hot EtOH (10 mL×3). The filtrate was concentrated to give 4-bromo-N¹-(isoquinolin-6-yl)benzene-1,2-diamine (180 mg, crude) as a yellow solid.

### Step 3. Synthesis of 6-(5-bromo-1H-benzo[d]imidazol-1-yl)isoquinoline

To a solution of 4-bromo-N1-(isoquinolin-6-yl)benzene-1,2-diamine (180 mg, 0.573 mmol) in trimethoxymethane (6.22 g, 58.6 mmol) was added PPTS (15 mg, 0.057 mmol). The mixture was stirred at 90° C. for 16 hours. The reaction mixture was concentrated and the residue was purified by Combi Flash (0% to 100% EtOAc in PE) to give 6-(5-bromo-1H-benzo[d]imidazol-1-yl)isoquinoline (170 mg, yield: 92%) as a yellow solid. ¹H NMR (400 MHz, DMSO-d₆) δ 7.54 (1H, dd, J=8.8, 2.0 Hz), 7.80 (1H, d, J=8.8 Hz), 7.98 (1H, d, J=5.6 Hz), 8.02-8.07 (2H, m), 8.35 (1H, d, J=2.0 Hz), 8.42 (1H, d, J=8.8 Hz), 8.62 (1H, d, J=5.6 Hz), 8.83 (1H, s), 9.46 (1H, s).

### Step 4. Synthesis of 6-(5-(4,4,5,5-tetramethyl-1,3,2-dioxaborolan-2-yl)-1H-benzo[d]imidazol-1-yl)isoquinoline

A mixture of 6-(5-bromo-1H-benzo[d]imidazol-1-yl)isoquinoline (170 mg, 0.524 mmol), Bis-Pin (160 mg, 0.630 mmol), Pd₂(dba)₃ (48 mg, 0.052 mmol), PCy₃ (30 mg, 0.11 mmol) and KOAc (103 mg, 1.05 mmol) in 1,4-dioxane (5 mL) was degassed and purged with N₂ for 3 times, then the mixture was stirred at 100° C. for 2 hours under N₂ atmosphere. The reaction mixture was filtered and the filtrate was concentrated to give 6-(5-(4,4,5,5-tetramethyl-1,3,2-dioxaborolan-2-yl)-1H-benzo[d]imidazol-1-yl)isoquinoline (194 mg, crude) as a yellow solid.

### Step 5. Synthesis of tert-butyl 8-(1-(isoquinolin-6-yl)-1H-benzo[d]imidazol-5-yl)-6-methyl-5-oxo-3,4,5,6-tetrahydro-2,6-naphthyridine-2(1H)-carboxylate

A mixture of 6-(5-(4,4,5,5-tetramethyl-1,3,2-dioxaborolan-2-yl)-1H-benzo[d]imidazol-1-yl)isoquinoline (194 mg, 0.553 mmo), tert-butyl 8-bromo-6-methyl-5-oxo-3,4,5,6-tetrahydro-2,6-naphthyridine-2(1H)-carboxylate (162 mg, 0.470 mmol), XPhos-Pd-G₃ (45 mg, 0.052 mmol) and K₂CO₃ (145 mg, 1.05 mmol) in 1,4-dioxane (8 mL) and H₂O (1 mL) was degassed and purged with N₂ for 3 times, then the mixture was stirred at 100° C. for 16 hours under N₂ atmosphere. The reaction mixture was quenched by addition H₂O (25 mL) and extracted with EtOAc (25 mL×3). The combined organic layers were dried over anhydrous Na₂SO₄, filtered and concentrated. The residue was purified by Combi Flash (0% to 10% MeOH in DCM) to give the title compound (240 mg, yield: 90%) as yellow oil.

### Example 55

### 6-(7-(tetrahydro-2H-pyran-4-yl)imidazo[1,2-b]pyridazin-3-yl)isoquinoline

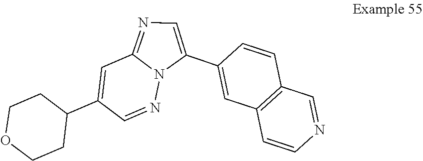 [SMILES: c1cc2cc(-c3cnc4cc(C5CCOCC5)cnn34)ccc2cn1]

A mixture of Example 54 (40 mg, 0.12 mmol), 10% Pd/C (20 mg) in MeOH (5 mL) was degassed and purged with H₂ for 3 times, and then the mixture was stirred at 20° C. for 12 hours under H₂ atmosphere (15 Psi). The reaction mixture was filtered and the filtrate was concentrated. The residue was purified by prep-HPLC (0.225% FA as an additive; Method C), then lyophilized to afford the title compound (7.06 mg, yield: 17%) as a yellow solid.

¹H NMR (400 MHz, DMSO-d₆) δ 1.76-1.90 (4H, m), 2.94-3.08 (1H, m), 3.44-3.52 (2H, m), 3.99-4.01 (2H, m), 7.87 (1H, d, J=6.0 Hz), 8.08 (1H, d, J=2.0 Hz), 8.22 (1H, d, J=8.8 Hz), 8.38 (1H, dd, J=8.8, 1.6 Hz), 8.48 (1H, s), 8.53 (1H, d, J=5.6 Hz), 8.80 (1H, d, J=2.0 Hz), 8.89 (1H, s), 9.31 (1H, s).

### Examples 56 and 57

### (1r,4r)-4-((6-(2-((tetrahydro-2H-pyran-4-yl)ethynyl)thiazol-5-yl)isoquinolin-4-yl)oxy)cyclohexane-1-carboxylic acid (Ex. 56) and (1s,4s)-4-((6-(2-((tetrahydro-2H-pyran-4-yl)ethynyl)thiazol-5-yl)isoquinolin-4-yl)oxy)cyclohexane-1-carboxylic acid (Ex. 57)

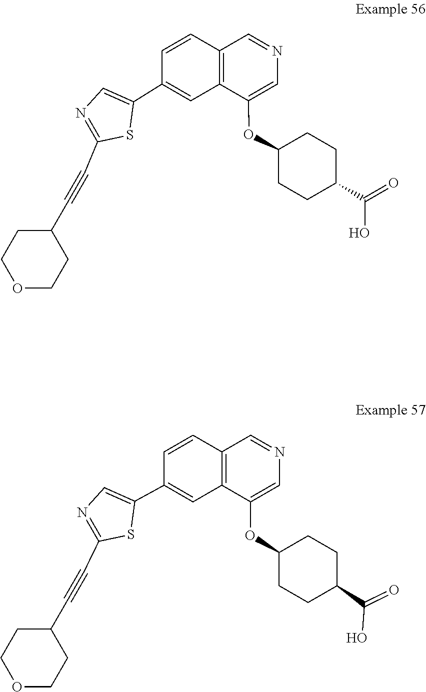 [SMILES: O=C(O)[C@H]1CC[C@H](Oc2cncc3ccc(-c4cnc(C#CC5CCOCC5)s4)cc23)CC1 ; O=C(O)[C@H]1CC[C@@H](Oc2cncc3ccc(-c4cnc(C#CC5CCOCC5)s4)cc23)CC1]

### Step 1. Synthesis of ethyl 4-((6-(4,4,5,5-tetramethyl-1,3,2-dioxaborolan-2-yl)isoquinolin-4-yl)oxy)cyclohexane-1-carboxylate

A mixture of compound Int-21 (770 mg, 2.04 mmol), Bis-Pin (620 mg, 2.44 mmol), Pd(dppf)Cl₂ (149 mg, 0.204 mmol) and KOAc (400 mg, 4.07 mmol) in 1,4-dioxane (20 mL) was degassed and purged with N₂ for 3 times, and then the mixture was stirred at 80° C. for 12 hours under N₂ atmosphere. The reaction mixture was concentrated and purified by Combi Flash (0% to 100% EtOAc in PE) to give ethyl 4-((6-(4,4,5,5-tetramethyl-1,3,2-dioxaborolan-2-yl)isoquinolin-4-yl)oxy)cyclohexane-1-carboxylate (550 mg, yield: 64%) as colorless oil.

### Step 2. Synthesis of ethyl 4-((6-(2-((tetrahydro-2H-pyran-4-yl)ethynyl)thiazol-5-yl)isoquinolin-4-yl)oxy)cyclohexane-1-carboxylate

A mixture of ethyl 4-((6-(4,4,5,5-tetramethyl-1,3,2-dioxaborolan-2-yl)isoquinolin-4-yl)oxy)cyclohexane-1-carboxylate (375 mg, 0.882 mmol), 5-bromo-2-((tetrahydro-2H-pyran-4-yl)ethynyl)thiazole (200 mg, 0.735 mmol), Pd(dppf)Cl₂ (48 mg, 0.074 mmol) and Na₂CO₃ (156 mg, 1.47 mmol) in 1,4-dioxane (10 mL) and H₂O (1 mL) was degassed and purged with N₂ for 3 times, and then the mixture was stirred at 90° C. for 12 hours under N₂ atmosphere. The reaction mixture was concentrated and the residue was purified by Combi Flash (0% to 100% EtOAc in PE) to give ethyl 4-((6-(2-((tetrahydro-2H-pyran-4-yl)ethynyl)thiazol-5-yl)isoquinolin-4-yl)oxy)cyclohexane-1-carboxylate (100 mg, yield: 28%) as yellow gum.

### Step 3. Synthesis of (cis/trans)-4-((6-(2-((tetrahydro-2H-pyran-4-yl)ethynyl)thiazol-5-yl)isoquinolin-4-yl)oxy)cyclohexane-1-carboxylic acid

To a solution of ethyl 4-((6-(2-((tetrahydro-2H-pyran-4-yl)ethynyl)thiazol-5-yl)isoquinolin-4-yl)oxy)cyclohexane-1-carboxylate (100 mg, 0.204 mmol) in MeOH (4 mL) and THF (4 mL) was added LiOH·H₂O (86 mg, 2.0 mmol) in H₂O (1 mL). The mixture was stirred at 25° C. for 2 hours. The reaction mixture was concentrated. The residue was acidified with 1N aqueous HCl to pH=3 and purified by prep-HPLC (0.05% HCl as an additive) then lyophilized to give Example 56 (7.37 mg, yield: 7.66%, peak 1) as a yellow solid and Example 57 (15.38 mg, yield: 16.23%, peak 2) as a yellow solid.

Example 56: ¹H NMR (400 MHz, DMSO-d₆) δ 1.62-1.70 (2H, m), 1.71-1.79 (4H, m), 1.80-1.84 (1H, m), 1.85-1.94 (4H, m), 1.98-2.05 (2H, m), 2.99-3.11 (1H, m), 3.41-3.52 (2H, m), 3.76-3.87 (2H, m), 4.90-4.96 (1H, m), 8.09 (1H, dd, J=8.4, 1.6 Hz), 8.17-8.20 (1H, m), 8.25 (1H, s), 8.29 (1H, s), 8.51 (1H, s), 8.93 (1H, s), 12.19 (1H, brs).

Example 57: ¹H NMR (400 MHz, DMSO-d₆) δ 1.60-1.72 (6H, m), 1.87-1.95 (2H, m), 2.00-2.07 (2H, m), 2.20-2.25 (2H, m), 2.35-2.40 (1H, m), 3.05-3.13 (1H, m), 3.46-3.51 (2H, m), 3.80-3.85 (2H, m), 4.70-4.90 (1H, m), 8.30 (1H, d, J=8.4 Hz), 8.35 (1H, s), 8.44 (1H, d, J=9.2 Hz), 8.48 (1H, s), 8.66 (1H, s), 9.30 (1H, s).

The following compounds were synthesized analogously to Examples 56 and 57

| Example | | | |
|---|---|---|---|
| No. | Structure | Name | ¹H NMR (400 MHz) |
| 58 | 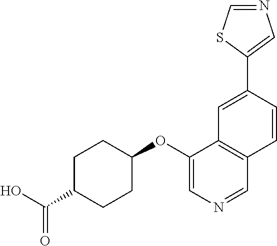 | (1r,4r)-4-((6- (thiazol-5- yl)isoquinolin-4- yl)oxy)cyclohexane- 1-carboxylic acid | DMSO-d₆: δ 1.57-1.67 (4H, m), 1.97-2.04 (2H, m), 2.19- 2.27 (2H, m), 2.34-2.41 (1H, m), 4.57-4.80 (1H, m), 8.09 (1H, dd, J = 8.8, 2.0 Hz), 8.16- 8.20 (1H, m), 8.23-8.25 (1H, m), 8.32 (1H, s), 8.56 (1H, s), 8.93 (1H, s), 9.21 (1H, s). |
| 59 | 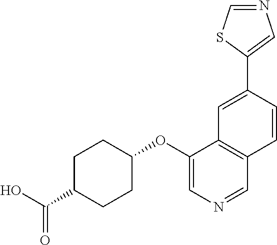 | (1s,4s)-4-((6- (thiazol-5- yl)isoquinolin-4- yl)oxy)cyclohexane- 1-carboxylic acid | DMSO-d₆; δ 1.70-1.91 (7H, m), 1.97-2.07 (2H, m), 4.92- 4.96 (1H, m), 8.09 (1H, dd, J = 8.8, 2.0 Hz), 8.16-8.20 (1H, m), 8.26-8.30 (2H, m), 8.53 (1H, s), 8.92 (1H, s), 9.20 (1H, s). |
| 67 | 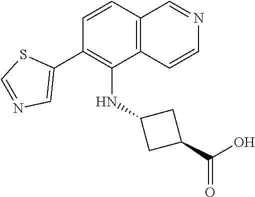 [SMILES: O=C(O)[C@H]1C[C@H](Nc2c(-c3cncs3)ccc3cnccc23)C1] | (1r,3r)-3-((6- (thiazol-5- yl)isoquinolin-5- yl)amino)cyclobutane- 1-carboxylic acid | DMSO-d₆; δ 2.16-2.25 (2H, m), 2.29-2.37 (2H, m), 2.79- 2.90 (1H, m), 3.57-3.72 (1H, m), 5.62 (1H, d, J = 10.4 Hz), 7.76 (1H, d, J = 8.8 Hz), 7.87 (1H, d, J = 8.4 Hz), 8.24 (1H, d, J = 6.0 Hz), 8.41 (1H, s), 8.55 (1H, d, J = 6.0 Hz), 9.15 (1H, s), 9.24 (1H, s), 12.10 (1H, brs). |
| 68 | 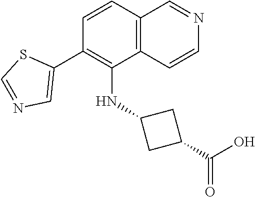 [SMILES: O=C(O)[C@H]1C[C@@H](Nc2c(-c3cncs3)ccc3cnccc23)C1] | (1s,3s)-3-((6- (thiazol-5- yl)isoquinolin-5- yl)amino)cyclobutane- 1-carboxylic acid | DMSO-d₆: 2.18-2.25 (4H, m), 3.24-3.40 (2H, m), 5.50- 5.60 (1H, m), 7.77 (1H, d, J = 8.4 Hz), 7.89 (1H, d, J = 8.8 Hz), 8.22 (1H, d, J = 6.0 Hz), 8.42 (1H, s), 8.54 (1H, d, J = 6.0 Hz), 9.15 (1H, s), 9.24 (1H, s), 12.17 (1H, brs). |
| 69 | 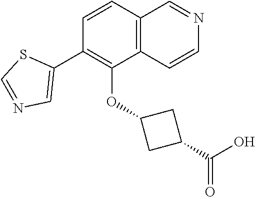 [SMILES: O=C(O)[C@H]1C[C@@H](Oc2c(-c3cncs3)ccc3cnccc23)C1] | (1s,3s)-3-((6- (thiazol-5- yl)isoquinolin-5- yl)oxy)cyclobutane- 1-carboxylic acid | DMSO-d₆; δ 2.34-2.41 (4H, m), 2.42-2.47 (1H, m), 4.16- 4.62 (1H, m), 7.93 (1H, d, J = 6.0 Hz), 8.00 (1H, d, J = 8.4 Hz), 8.09 (1H, d, J = 8.8 Hz), 8.51-8.65 (2H, m), 9.25 (1H, s), 9.35 (1H, s), 12.28 (1H, brs). |

### Example 61

### 6-(3-((tetrahydro-2H-pyran-4-yl)ethynyl)-1H-1,2,4-triazol-5-yl)isoquinoline

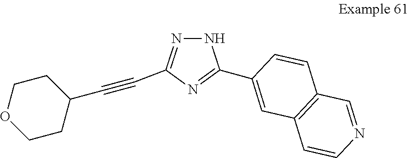 [SMILES: C(#CC1CCOCC1)c1n[nH]c(-c2ccc3cnccc3c2)n1]

### Step 1. Synthesis of 3,5-dibromo-1-((2-(trimethylsilyl)ethoxy)methyl)-1H-1,2,4-triazole

To a solution of 3,5-dibromo-1H-1,2,4-triazolea (500 mg, 2.20 mmol) in DCM (6 mL) was added Et₃N (0.9 mL) and SEM-Cl (551 mg, 3.31 mmol). The mixture was stirred at 25° C. for 3 hours. The reaction mixture was concentrated and the residue was purified by silica gel column (PE/EtOAc=2/1) to afford 3,5-dibromo-1-((2-(trimethylsilyl)ethoxy)methyl)-1H-1,2,4-triazole (500 mg, yield: 46%) as brown oil.

### Step 2. Synthesis of 6-(3-bromo-1-((2-(trimethylsilyl)ethoxy)methyl)-1H-1,2,4-triazol-5-yl)isoquinoline

A mixture of 3,5-dibromo-1-((2-(trimethylsilyl)ethoxy)methyl)-1H-1,2,4-triazole (400 mg, 1.12 mmol), compound Int-1 (314 mg, 1.23 mmol), Pd(dppf)Cl₂ (82 mg, 0.11 mmol) and Na₂CO₃ (237 mg, 2.24 mmol) in 1, 4-dioxane (6 mL) and H₂O (2 mL) was degassed and purged with N₂ for 3 times, then the mixture was stirred at 90° C. for 16 hours under N₂ atmosphere. The reaction mixture was concentrated and the residue was purified by silica gel column (PE/EtOAc=1/1) to afford 6-(3-bromo-1-((2-(trimethylsilyl)ethoxy)methyl)-1H-1,2,4-triazol-5-yl)isoquinoline (240 mg, yield: 32%) as brown oil.

### Step 3. Synthesis of 6-(3-((tetrahydro-2H-pyran-4-yl)ethynyl)-1-((2-(trimethylsilyl)ethoxy)methyl)-1H-1,2,4-triazol-5-yl)isoquinoline

A mixture of 6-(3-bromo-1-((2-(trimethylsilyl)ethoxy)methyl)-1H-1,2,4-triazol-5-yl)isoquinoline (240 mg, 0.592 mmol), Pd(PPh₃)₂Cl₂ (42 mg, 0.06 mmol), CuI (23 mg, 0.12 mmol) and Et₃N (299 mg, 2.96 mmol) in DMF (5 mL) was degassed and purged with N₂ for 3 times, then 4-ethynyltetrahydropyran (130 mg, 1.18 mmol) was added to the reaction mixture and stirred at 80° C. for 2 hours under N₂ atmosphere. The reaction mixture was concentrated and the residue was purified by silica gel column (PE/EtOAc=1/1) to afford 6-(3-((tetrahydro-2H-pyran-4-yl)ethynyl)-1-((2-(trimethylsilyl)ethoxy)methyl)-1H-1,2,4-triazol-5-yl)isoquinoline (176 mg, yield: 40%) as brown oil.

### Step 4. Synthesis of (5-(isoquinolin-6-yl)-3-((tetrahydro-2H-pyran-4-yl)ethynyl)-1H-1,2,4-triazol-1-yl)methanol

A solution of 6-(3-((tetrahydro-2H-pyran-4-yl)ethynyl)-1-((2-(trimethylsilyl)ethoxy)methyl)-1H-1,2,4-triazol-5-yl)isoquinoline (126 mg, 0.290 mmol) in DCM (4 mL) was added TFA (1 mL). The mixture was stirred at 25° C. for 6 hours. The reaction mixture was concentrated to give (5-(isoquinolin-6-yl)-3-((tetrahydro-2H-pyran-4-yl)ethynyl)-1H-1,2,4-triazol-1-yl)methanol (140 mg, crude, TFA salt) as a brown solid.

### Step 5. Synthesis of 6-(3-((tetrahydro-2H-pyran-4-yl)ethynyl)-1H-1,2,4-triazol-5-yl)isoquinoline

A mixture of (5-(isoquinolin-6-yl)-3-((tetrahydro-2H-pyran-4-yl)ethynyl)-1H-1,2,4-triazol-1-yl)methanol (140 mg, 0.419 mmol) in MeOH (2 mL) was added 28% aqueous NH₃·H₂O (2 mL). The mixture was stirred at 25° C. for 2 hours. The reaction mixture was concentrated and the residue was purified by prep-HPLC (0.05% NH₄HCO₃ as an additive), then lyophilized to afford the title compound (45 mg, yield: 36%) as a white solid.

¹H NMR (400 MHz, DMSO-d₆) δ 1.57-1.73 (2H, m), 1.84-1.95 (2H, m), 2.96-3.05 (1H, m), 3.45-3.51 (2H, m), 3.79-3.88 (2H, m), 7.93 (1H, d, J=5.6 Hz), 8.23 (1H, d, J=8.8 Hz), 8.25-8.30 (1H, m), 8.55 (1H, d, J=5.6 Hz), 8.60 (1H, s), 9.35 (1H, s).

### Example 62

### 5-(3-methylisoquinolin-6-yl)-2-((tetrahydro-2H-pyran-4-yl)ethynyl)thiazole

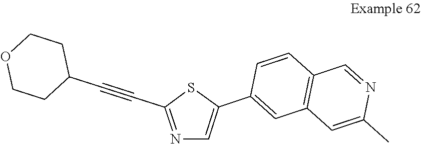 [SMILES: Cc1cc2cc(-c3cnc(C#CC4CCOCC4)s3)ccc2cn1]

### Step 1. Synthesis of 2-bromo-5-(3-methylisoquinolin-6-yl)thiazole

To a solution of compound isoamyl nitrite (73 mg, 0.62 mmol) and CuBr₂ (185 mg, 0.829 mmol) in DMF (2 mL) was added compound Int-12 (100 mg, 0.414 mmol) and the mixture was stirred at 0° C. for 0.5 hour. The resulting mixture was heated at 50° C. for 1 hour and diluted with water (30 mL), then extracted with EtOAc (30 mL×3). The combined organic layers were dried over anhydrous Na₂SO₄, filtered and concentrated. Then the residue was purified by silica gel column (PE/EtOAc=2/1) to afford 2-bromo-5-(3-methylisoquinolin-6-yl)thiazole (100 mg, yield: 70%) as a yellow solid.

¹H NMR (400 MHz, DMSO-d₆) δ 2.62 (3H, s), 7.67 (1H, s), 7.92 (1H, dd, J=8.4, 1.6 Hz), 8.11 (1H, s), 8.16 (1H, d, J=8.4 Hz), 8.34 (1H, s), 9.24 (1H, s).

### Step 2. Synthesis of 5-(3-methylisoquinolin-6-yl)-2-((tetrahydro-2H-pyran-4-yl)ethynyl)thiazole

A mixture of 2-bromo-5-(3-methylisoquinolin-6-yl)thiazole (50 mg, 0.16 mmol), Pd(PPh₃)₂Cl₂ (12 mg, 0.016 mmol), CuI (6 mg, 0.03 mmol) and Et₃N (99 mg, 0.98 mmol) in THE (3 mL) was degassed and purged with N₂ for 3 times. Then 4-ethynyltetrahydro-2H-pyran (27 mg, 0.25 mmol) was added and the resulting mixture was stirred at 40° C. for 16 hours under N₂ atmosphere. The reaction mixture was diluted with water (30 mL) and extracted with EtOAc (30 mL×3). The combined organic layers were dried over anhydrous Na₂SO₄, filtered and concentrated. The residue was purified by silica gel column (EtOAc as eluent) and further purified by prep-HPLC (0.225% FA as an additive; Method C), then lyophilized to afford the title compound (11 mg, yield: 13%) as an off-white solid.

¹H NMR (400 MHz, DMSO-d₆) δ 1.61-1.73 (2H, m), 1.86-1.94 (2H, m), 2.62 (3H, s), 3.02-3.10 (1H, m), 3.43-3.52 (2H, m), 3.70-3.93 (2H, m), 7.66 (1H, s), 7.95 (1H, dd, J=8.8, 1.2 Hz), 8.12-8.18 (2H, m), 8.50 (1H, s), 9.23 (1H, s).

### Example 64

### 3-fluoro-4-methyl-6-(1-methyl-1H-pyrazol-4-yl)isoquinoline

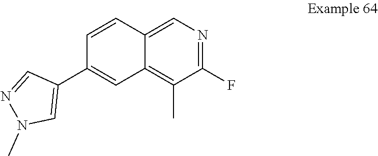 [SMILES: Cc1c(F)ncc2ccc(-c3cnn(C)c3)cc12]

### Step 1. Synthesis of 4-methyl-6-(1-methyl-1H-pyrazol-4-yl)isoquinolin-3-amine

A mixture of 4-iodo-6-(1-methyl-1H-pyrazol-4-yl)isoquinolin-3-amine (500 mg, 1.43 mmol), 2,4,6-trimethyl-1,3,5,2,4,6-trioxatriborinane (717 mg, 2.86 mmol, 50% in toluene), Pd(dppf)Cl₂ (105 mg, 0.143 mmol) and K₃PO₄ (606 mg, 2.86 mmol) in 1, 4-dioxane (10 mL) was degassed and purged with N₂ for 3 times, and stirred at 90° C. for 16 hours under N₂ atmosphere. The reaction mixture was filtered and the filtrate was concentrated. The residue was purified by flash silica gel chromatography (DCM/MeOH=10/1) to afford 4-methyl-6-(1-methyl-1H-pyrazol-4-yl)isoquinolin-3-amine (220 mg, yield: 65%) as a yellow solid.

### Step 2. Synthesis of 3-fluoro-4-methyl-6-(1-methyl-1H-pyrazol-4-yl)isoquinoline

A solution of NaNO₂ (232 mg, 3.36 mmol) in H₂O (5 mL) was added slowly to a mixture of 4-methyl-6-(1-methyl-1H-pyrazol-4-yl)isoquinolin-3-amine (160 mg, 0.671 mmol) in 70% HF/pyridine (11.0 g, 77.7 mmol) at 0° C. and stirred at 0° C. for 0.5 hour. Then the reaction mixture was stirred at 20° C. for another 1 hour. The reaction mixture was basified with saturated aqueous Na₂CO₃ to pH=9 and extracted with DCM (20 mL×3). The combined organic layer was dried over anhydrous Na₂SO₄, filtered and concentrated. The residue was purified by prep-HPLC (10 mM NH₄HCO₃ as an additive), then lyophilized to afford the title compound (29.2 mg, yield: 18%) as a white solid.

¹H NMR (400 MHz, CDCl₃) δ 2.59 (3H, s), 4.02 (3H, s), 7.66 (1H, dd, J=8.4, 1.6 Hz), 7.82 (1H, s), 7.95-8.05 (3H, m), 8.76 (1H, s).

### Examples 65 and 66

### (1r,3r)-3-((6-(2-methyloxazol-5-yl)isoquinolin-5-yl)amino)cyclobutane-1-carboxylic acid (Ex. 65) and (1s,3s)-3-((6-(2-methyloxazol-5-yl)isoquinolin-5-yl)amino)cyclobutane-1-carboxylic acid (Ex. 66)

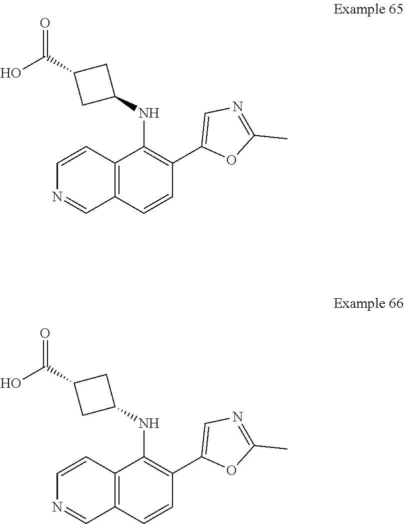 [SMILES: Cc1ncc(-c2ccc3cnccc3c2N[C@H]2C[C@H](C(=O)O)C2)o1 ; Cc1ncc(-c2ccc3cnccc3c2N[C@H]2C[C@@H](C(=O)O)C2)o1]

### Step 1. Synthesis of ethyl 3-((6-(2-methyloxazol-5-yl)isoquinolin-5-yl)amino)cyclobutane-1-carboxylate

To a solution of compound Int-23 (280 mg, 1.24 mmol) and ethyl 3-oxocyclobutane-1-carboxylate (353 mg, 2.49 mmol) in DCM (30 mL) was added TiCl₄ (1.89 g, 9.94 mmol) at 0° C., the mixture was stirred at 45° C. for 16 hours. NaBH₃CN (273 mg, 4.35 mmol) was added to reaction mixture at 20° C., the reaction mixture was stirred at 20° C. for 3 hours and stirred at 45° C. for 16 hours to give yellow suspension. The reaction mixture was diluted with DCM (40 mL) then poured into saturated aqueous NaHCO₃ (100 mL) was added the reaction mixture. The mixture was filtered and the solid was washed with DCM (50 mL×2). The filtrate was separated and extracted with DCM (80 mL). The combined organic layer was dried over anhydrous Na₂SO₄, filtered and concentrated. The residue was purified by flash silica gel chromatography (ISCO®; 20 g SepaFlash® Silica Flash Column, Eluent of ˜2% MeOH/DCM gradient @40 mL/min) to give ethyl 3-((6-(2-methyloxazol-5-yl)isoquinolin-5-yl)amino)cyclobutane-1-carboxylate (160 mg, yield: 30%) as a yellow solid.

### Step 2. Synthesis of (cis/trans)-3-((6-(2-methyloxazol-5-yl)isoquinolin-5-yl)amino)cyclobutane-1-carboxylic acid

To a solution of ethyl 3-((6-(2-methyloxazol-5-yl)isoquinolin-5-yl)amino)cyclobutane-1-carboxylate (160 mg, 0.455 mmol) in THF (3 mL) and H₂O (3 mL) was added LiOH·H₂O (96 mg, 2.3 mmol), the mixture was stirred at 20° C. for 2 hours to give yellow solution. The reaction mixture was acidified with 1N aqueous HCl to pH=5 and concentrated. The residue was purified by prep-HPLC (0.05% HCl as an additive) and lyophilized to give Example 65 (28 mg, yield: 19%) as a yellow solid and Example 66 (87 mg, yield: 59%) as a yellow solid.

Example 65: ¹H NMR (400 MHz, DMSO-d₆) δ 2.30-2.39 (2H, m), 2.40-2.47 (2H, m), 2.56 (3H, s), 2.85-2.96 (1H, m), 3.81-3.93 (1H, m), 7.68 (1H, s), 8.07-8.15 (2H, m), 8.69 (1H, d, J=6.8 Hz), 8.79 (1H, d, J=6.8 Hz), 9.78 (1H, s).

Example 66: ¹H NMR (400 MHz, DMSO-d₆) δ 2.30-2.41 (4H, m), 2.55-2.62 (4H, m), 3.50-3.68 (1H, m), 7.72 (1H, s), 8.08-8.18 (2H, m), 8.69 (1H, d, J=6.8 Hz), 8.76 (1H, d, J=6.8 Hz), 9.77 (1H, s).

The following compound was synthesized analogously to Examples 65 and 66

| Example | | | |
|---|---|---|---|
| No. | Structure | Name | ¹H NMR (400 MHz) |
| 91 | 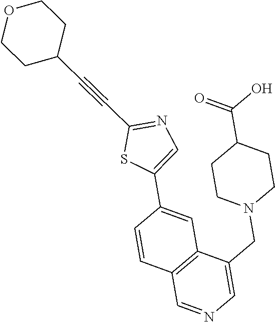 [SMILES: O=C(O)C1CCN(Cc2cncc3ccc(-c4cnc(C#CC5CCOCC5)s4)cc23)CC1] | 1-((6-(2-((tetrahydro- 2H-pyran-4- yl)ethynyl)thiazol-5- yl)isoquinolin-4- yl)methyl)piperidine- 4-carboxylic acid | DMSO-d₆; δ 1.42-1.57 (2H, m), 1.62-1.74 (2H, m), 1.80-1.85 (2H, m), 1.86-1.96 (2H, m), 2.10-2.15 (2H, m), 2.19-2.29 (1H, m), 2.80- 2.90 (2H, m), 3.03-3.12 (1H, m), 3.42-3.53 (2H, m), 3.79-3.86 (2H, m), 3.88 (2H, s), 8.08 (1H, d, J = 8.4 Hz), 8.21 (1H, d, J = 8.8 Hz), 8.42 (1H, s), 8.50-8.58 (2H, m), 9.25 (1H, s), 12.12 (1H, brs). |

### Example 71

### (1s,3s)-3-((5-(isoquinolin-6-yl)thiazol-2-yl)amino)cyclobutane-1-carboxylic acid

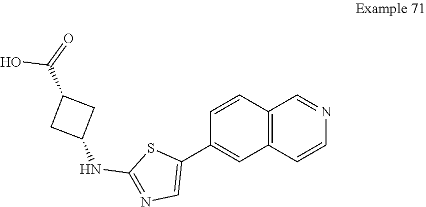 [SMILES: O=C(O)[C@H]1C[C@@H](Nc2ncc(-c3ccc4cnccc4c3)s2)C1]

### Step 1. Synthesis of methyl (1s,3s)-3-((5-bromothiazol-2-yl)(tert-butoxycarbonyl)amino)cyclobutane-1-carboxylate

A mixture of tert-butyl (5-bromothiazol-2-yl)carbamate (900 mg, 3.22 mmol), methyl (1r,3r)-3-hydroxycyclobutane-1-carboxylate (671 mg, 5.16 mmol), PPh₃ (1.69 g, 6.45 mmol) in THF (10 mL) was degassed and purged with N₂ for 3 times, then the DIAD (1.04 g, 5.16 mmol) was added at 0° C. and the mixture was stirred at 80° C. for 3 hours under N₂ atmosphere. The reaction mixture was concentrated and the residue was purified by silica gel column (PE/EtOAc=5/1) to afford methyl (1s,3s)-3-((5-bromothiazol-2-yl)(tert-butoxycarbonyl)amino)cyclobutane-1-carboxylate (895 mg, yield: 71%) as yellow oil.

### Step 2. Synthesis of methyl (1s,3s)-3-((tert-butoxycarbonyl)(5-(isoquinolin-6-yl)thiazol-2-yl)amino)cyclobutane-1-carboxylate

A mixture of methyl (1s,3s)-3-((5-bromothiazol-2-yl)(tert-butoxycarbonyl)amino)cyclobutane-1-carboxylate (150 mg, 0.383 mmol), compound Int-1 (117 mg, 0.460 mmol), Pd(dtbpf)Cl₂ (25 mg, 0.38 mmol) and Na₂CO₃ (81 mg, 0.77 mmol) in 1, 4-dioxane (3 mL) and H₂O (0.6 mL) was degassed and purged with N₂ for 3 times and the mixture was stirred at 90° C. for 1.5 hours under N₂ atmosphere. The reaction mixture was concentrated and the residue was purified by silica gel column (PE/EtOAc=1/1) to afford methyl (1s,3s)-3-((tert-butoxycarbonyl)(5-(isoquinolin-6-yl)thiazol-2-yl)amino)cyclobutane-1-carboxylate (130 mg, yield: 77%) as brown oil. ¹H NMR (400 MHz, DMSO-d₆) δ 1.56 (9H, s), 2.45-2.48 (2H, m), 2.69-2.81 (2H, m), 2.90-3.02 (1H, m), 3.61 (3H, s), 5.08-5.22 (1H, m), 7.83 (1H, d, J=5.6 Hz), 8.03 (1H dd, J=8.8, 1.6 Hz), 8.11-8.17 (3H, m), 8.50 (1H, d, J=5.6 Hz), 9.27 (1H, s).

### Step 3. Synthesis of methyl (1s,3s)-3-((5-(isoquinolin-6-yl)thiazol-2-yl)amino)cyclobutane-1-carboxylate

To a solution of methyl (1s,3s)-3-((tert-butoxycarbonyl)(5-(isoquinolin-6-yl)thiazol-2-yl)amino)cyclobutane-1-carboxylate (130 mg, 0.295 mmol) in DCM (2 mL) was added TFA (2 mL) and stirred at 20° C. for 1.5 hours. The reaction mixture was concentrated and then basified with saturated aqueous NaHCO₃ to pH=8 and extracted with DCM (20 mL×3). The combined organic layers were dried anhydrous Na₂SO₄, filtered and concentrated to afford methyl (1s,3s)-3-((5-(isoquinolin-6-yl)thiazol-2-yl)amino)cyclobutane-1-carboxylate (92 mg, crude) as a yellow solid.

### Step 4. Synthesis of (1s,3s)-3-((5-(isoquinolin-6-yl)thiazol-2-yl)amino)cyclobutane-1-carboxylic acid

A mixture of methyl (1s,3s)-3-((5-(isoquinolin-6-yl)thiazol-2-yl)amino)cyclobutane-1-carboxylate (92 mg, 0.27 mmol) in THF (2 mL), MeOH (2 mL) and H₂O (1 mL) was added LiOH·H₂O (114 mg, 2.71 mmol). The mixture was stirred at 20° C. for 1.5 hours. The reaction mixture was concentrated and the residue was acidified with 1N aqueous HCl to pH=5 and filtered. The solid was further purified by prep-HPLC (0.05% NH₄OAc as an additive), then lyophilized to afford the title compound (35.79 mg, yield: 40%) as a yellow solid. ¹H NMR (400 MHz, DMSO-d₆) δ 2.06-2.18 (2H, m), 2.57-2.65 (2H, m), 2.75-2.84 (1H, m), 4.05-4.17 (1H, m), 7.73-7.81 (3H, m), 7.90 (1H, dd, J=8.4, 1.6 Hz), 8.04 (1H, d, J=8.8 Hz), 8.35 (1H, d, J=7.2 Hz), 8.44 (1H, d, J=5.6 Hz), 9.19 (1H, brs).

The following compound was synthesized analogously to Example 71

| Example | | | |
|---|---|---|---|
| No. | Structure | Name | ¹H NMR (400 MHz) |
| 72 | 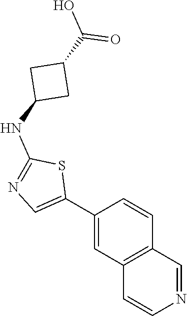 [SMILES: O=C(O)[C@H]1C[C@H](Nc2ncc(-c3ccc4cnccc4c3)s2)C1] | (1r,3r)-3-((5- (isoquinolin-6-yl)thiazol- 2-yl)amino)cyclobutane- 1-carboxylic acid | DMSO-d₆; δ 2.20-2.31 (2H, m), 2.53-2.59 (2H, m), 2.95-3.05 (1H, m), 4.22-4.34 (1H, m), 7.73-7.82 (3H, m), 7.92 (1H, dd, J = 8.8, 2.0 Hz), 8.04 (1H, d, J = 8.8 Hz), 8.38-8.46 (2H, m), 9.19 (1H, s), 12.36 (1H, brs). |

### Example 73

### 1-(3-(isoquinolin-6-yl)pyrazolo[1,5-a]pyrazin-6-yl)azetidine-3-carboxylic acid

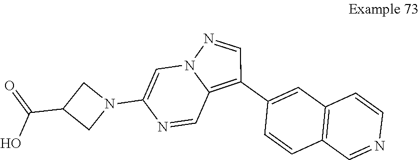 [SMILES: O=C(O)C1CN(c2cn3ncc(-c4ccc5cnccc5c4)c3cn2)C1]

### Step 1. Synthesis of 6-(6-chloropyrazolo[1,5-a]pyrazin-3-yl)isoquinoline

A mixture of 6-chloro-3-iodopyrazolo[1,5-a]pyrazine (500 mg, 1.79 mmol), compound Int-1 (548 mg, 2.16 mmol), Pd(dppf)Cl₂ (131 mg, 0.180 mmol) and Na₂CO₃ (569 mg, 5.38 mmol) in 1, 4-dioxane (10 mL) and H₂O (1 mL) was degassed and purged with N₂ for 3 times, and then the mixture was stirred at 90° C. for 1 hour under N₂ atmosphere. The reaction mixture was concentrated and the residue was purified by silica gel chromatography (DCM/MeOH=10/1) to afford 6-(6-chloropyrazolo[1,5-a]pyrazin-3-yl)isoquinoline (200 mg, yield: 40%) as a yellow solid. ¹H NMR (400 MHz, CDCl₃) δ 7.74 (1H, d, J=5.6 Hz), 7.89 (1H, dd, J=8.4, 1.6 Hz), 8.06 (1H, s), 8.14 (1H, d, J=8.4 Hz), 8.37 (1H, s), 8.56 (1H, d, J=1.2 Hz), 8.61 (1H, d, J=5.6 Hz), 9.24 (1H, d, J=1.2 Hz), 9.32 (1H, s).

### Step 2. Synthesis of ethyl 1-(3-(isoquinolin-6-yl)pyrazolo[1,5-a]pyrazin-6-yl)azetidine-3-carboxylate

A mixture of 6-(6-chloropyrazolo[1,5-a]pyrazin-3-yl)isoquinoline (140 mg, 0.499 mmol), ethyl azetidine-3-carboxylate hydrochloride (302 mg, 1.50 mmol), Pd₂(dba)₃ (46 mg, 0.050 mmol), RuPhos (47 mg, 0.099 mmol) and Cs₂CO₃ (812 mg, 2.49 mmol) in 1, 4-dioxane (5 mL) was degassed and purged with N₂ for 3 times, and then the mixture was stirred at 110° C. for 16 hours under N₂ atmosphere. The reaction mixture was concentrated and the residue was purified by silica gel chromatography (DCM/MeOH=10/1) to afford ethyl 1-(3-(isoquinolin-6-yl)pyrazolo[1,5-a]pyrazin-6-yl)azetidine-3-carboxylate (190 mg, yield: 76%) as a yellow solid.

### Step 3. Synthesis of 1-(3-(isoquinolin-6-yl)pyrazolo[1,5-a]pyrazin-6-yl)azetidine-3-carboxylic acid

To a solution of ethyl 1-(3-(isoquinolin-6-yl)pyrazolo[1,5-a]pyrazin-6-yl)azetidine-3-carboxylate (170 mg, 0.455 mmol) in THF (1.6 mL), MeOH (1.6 mL) and H₂O (0.8 mL) was added LiOH·H₂O (191 mg, 4.55 mmol) at 20° C. and the reaction mixture was stirred at 20° C. for 2 hours. To the reaction mixture was added HCOOH to adjust the pH=3, the precipitate was filtered, then triturated with DMF (3 mL) and washed with H₂O/CH₃CN (5 mL, 1/1), then lyophilized to afford the title compound (36.4 mg, yield: 22%) as a yellow solid. ¹H NMR (400 MHz, DMSO-d₆) δ 3.56-3.62 (1H, m), 3.99-4.05 (2H, m), 4.12-4.18 (2H, m), 7.89 (1H, d, J=6.0 Hz), 8.08 (1H, d, J=1.2 Hz), 8.10-8.14 (1H, m), 8.16-8.21 (1H, m), 8.37 (1H, s), 8.50 (1H, d, J=5.6 Hz), 8.54 (1H, s), 9.28 (1H, s), 9.47 (1H, d, J=1.2 Hz).

### Example 74

### 3-((6-(thiazol-5-yl)isoquinolin-3-yl)amino)cyclobutane-1-carboxylic acid

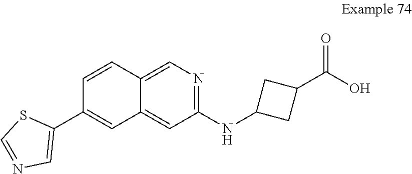 [SMILES: O=C(O)C1CC(Nc2cc3cc(-c4cncs4)ccc3cn2)C1]

### Step 1. Synthesis of ethyl 3-((6-bromoisoquinolin-3-yl)amino)cyclobutane-1-carboxylate

To a solution of 6-bromoisoquinolin-3-amine (500 mg, 2.24 mmol) and ethyl 3-oxocyclobutane-1-carboxylate (414 mg, 2.91 mmol) in DCM (20 mL) was added TiCl₄ (4.74 g, 25.0 mmol) dropwise at 0° C. After stirring at 0° C. for 6 hours, NaBH₃CN (141 mg, 2.24 mmol) was added at 0° C. and the resulting reaction mixture was stirred at 20° C. for another 6 hours. The reaction mixture was quenched with saturated aqueous NaHCO₃ to adjust to pH=8 and filtered. The filtrated was extracted with DCM (50 mL×3). The combined organic layer was dried over anhydrous Na₂SO₄, filtered and concentrated. The residue was purified by flash silica gel column (PE/EtOAc=3/1) to afford ethyl 3-((6-bromoisoquinolin-3-yl)amino)cyclobutane-1-carboxylate (422 mg, yield: 54%) as a green solid.

### Step 2. Synthesis of ethyl 3-((6-(thiazol-5-yl)isoquinolin-3-yl)amino)cyclobutane-1-carboxylate

A mixture of ethyl 3-((6-bromoisoquinolin-3-yl)amino)cyclobutane-1-carboxylate (300 mg, 0.859 mmol), 5-(4,4,5,5-tetramethyl-1,3,2-dioxaborolan-2-yl)thiazole (236 mg, 1.12 mmol), Na₂CO₃ (182 mg, 1.72 mmol), Pd(dtbpf)Cl₂ (56 mg, 0.086 mmol) in dioxane (8 mL) and H₂O (1.5 mL) was degassed and purged with N₂ for 3 times, and then the mixture was stirred at 90° C. for 16 hours under N₂ atmosphere. The reaction mixture was concentrated and the residue was diluted with water (30 mL), then extracted with EtOAc (50 mL×3). The combined organic layer was dried over anhydrous Na₂SO₄, filtered and concentrated. The residue was purified by flash silica gel column (PE/EtOAc=1/1) to afford ethyl 3-((6-(thiazol-5-yl)isoquinolin-3-yl)amino)cyclobutane-1-carboxylate (100 mg, yield: 33%) as a green solid.

### Step 3. Synthesis of 3-((6-(thiazol-5-yl)isoquinolin-3-yl)amino)cyclobutane-1-carboxylic acid

To a solution of ethyl 3-((6-(thiazol-5-yl)isoquinolin-3-yl)amino)cyclobutane-1-carboxylate (100 mg, 0.280 mmol) in THF (2 mL), H₂O (1 mL) and MeOH (2 mL) was added LiOH·H₂O (47 mg, 1.1 mmol,). The mixture was stirred at 20° C. for 2 hours. The reaction mixture was acidified with FA to pH=4, then concentrated. The residue was purified by prep-HPLC (0.225% FA as an additive; Method C) and lyophilized to afford the title compound (46.42 mg, yield: 48%, FA salt, cis/trans=1/1) as a yellow solid. ¹H NMR (400 MHz, DMSO-d₆) δ 2.07-2.15 (2H, m), 2.19-2.27 (2H, m), 2.56-2.62 (4H, m), 2.75-2.85 (1H, m), 2.97-3.05 (1H, m), 4.08-4.16 (1H, m), 4.23-4.30 (1H, m), 6.51 (1H, s), 6.57 (1H, s), 6.87-6.95 (2H, m), 7.50-7.52 (2H, m), 7.88-7.90 (4H, m), 8.44-8.47 (2H, m), 8.84-8.85 (2H, m), 9.15 (2H, s).

The following compound was synthesized analogously to Example 74

| Example | | | |
|---|---|---|---|
| No. | Structure | Name | ¹H NMR (400 MHz) |
| 109 | 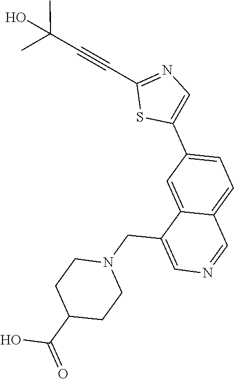 [SMILES: CC(C)(O)C#Cc1ncc(-c2ccc3cncc(CN4CCC(C(=O)O)CC4)c3c2)s1] | 1-((6-(2-(3-hydroxy- 3-methylbut-1-yn-1- yl)thiazol-5- yl)isoquinolin-4- yl)methyl)piperidine- 4-carboxylic acid | DMSO-d₆; δ 1.45-1.50 (1H, m), 1.52 (6H, s), 1.75-1.83 (2H, m), 2.08-2.16 (2H, m), 2.50-2.55 (2H, m), 2.80-2.85 (2H, m), 3.88 (2H, s), 5.82 (1H, s), 8.08 (1H, dd, J = 8.4, 1.6 Hz), 8.21 (1H, d, J = 8.4 Hz), 8.41 (1H, s), 8.52- 8.57 (2H, m), 9.25 (1H, s). |

### Example 75

### 5-(4-(azetidin-3-yloxy)-3-methylisoquinolin-6-yl)thiazole

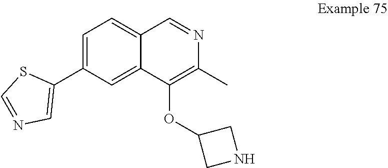 [SMILES: Cc1ncc2ccc(-c3cncs3)cc2c1OC1CNC1]

### Step 1. Synthesis of tert-butyl 3-((3-methyl-6-(thiazol-5-yl)isoquinolin-4-yl)oxy)azetidine-1-carboxylate

A mixture of compound Int-26 (600 mg, 1.53 mmol), 5-(4,4,5,5-tetramethyl-1,3,2-dioxaborolan-2-yl)thiazole (307 mg, 1.68 mmol), Pd(dtbpf)Cl₂ (149 mg, 0.230 mmol) and Na₂CO₃ (323 mg, 3.05 mmol) in dioxane (13 mL) and H₂O (2 mL) was degassed and purged with N₂ for 3 times, and then the mixture was stirred at 90° C. for 3 hours under N₂ atmosphere. The reaction mixture was concentrated and the residue was diluted with water (20 mL), then extracted with EtOAc (30 mL×3). The combined organic layer was dried over anhydrous Na₂SO₄, filtered and concentrated. The residue was purified by flash silica gel column (PE/EtOAc=1/4) to afford tert-butyl 3-((3-methyl-6-(thiazol-5-yl)isoquinolin-4-yl)oxy)azetidine-1-carboxylate (180 mg, yield: 28%) as brown oil. ¹H NMR (400 MHz, DMSO-d₆) δ 1.40 (9H, s), 2.56 (3H, s), 4.15-4.26 (4H, m), 4.87-4.96 (1H, m), 7.90-8.10 (1H, m), 8.09 (1H, s), 8.20 (1H, d, J=8.0 Hz), 8.60 (1H, s), 9.06 (1H, s), 9.22 (1H, s).

### Step 2. Synthesis of 5-(4-(azetidin-3-yloxy)-3-methylisoquinolin-6-yl)thiazole

To a solution of tert-butyl 3-((3-methyl-6-(thiazol-5-yl)isoquinolin-4-yl)oxy)azetidine-1-carboxylate (160 mg, 0.403 mmol) in DCM (5 mL) was added TFA (5 mL) and the reaction mixture was stirred at 25° C. for 2 hours. The reaction mixture was concentrated and the residue was quenched with 1 N aqueous NaOH (10 mL), then extracted with DCM (15 mL×3). The combined organic layer was dried over anhydrous Na₂SO₄, filtered and concentrated to give the title compound (95 mg, yield: 79%) as yellow gum, some of which was used directly for additional reactions; 20 mg were purified by prep-HPLC (0.05% NH₃H₂O+10 mM NH₄HCO₃ as an additive), then lyophilized to afford the title compound (10.32 mg) as a white solid. ¹H NMR (400 MHz, DMSO-d₆) δ 2.64 (3H, s), 3.70-3.83 (2H, m), 3.88-4.00 (2H, m), 4.83-4.94 (1H, m), 8.05-8.15 (1H, m), 8.17 (1H, s), 8.28 (1H, d, J=8.0 Hz), 8.67 (1H, s), 9.13 (1H, s), 9.31 (1H, s).

The following compounds were synthesized analogously to Example 75

| Example | | | |
|---|---|---|---|
| No. | Structure | Name | ¹H NMR (400 MHz) |
| 77 | 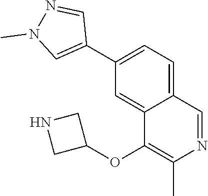 [SMILES: Cc1ncc2ccc(-c3cnn(C)c3)cc2c1OC1CNC1] | 4-(azetidin-3- yloxy)-3-methyl-6- (1-methyl-1H- pyrazol-4- yl)isoquinoline | DMSO-d₆: δ 1.40 (9H, s), 2.54 (3H, s), 3.91 (3H, s), 4.15-4.25 (4H, m), 4.82-4.98 (1H, m), 7.80-7.94 (1H, m), 7.99 (1H, s), 8.05-8.14 (2H, m), 8.42 (1H, s), 8.96 (1H, s). |
| 95 | 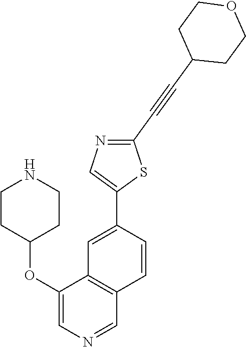 [SMILES: C(#CC1CCOCC1)c1ncc(-c2ccc3cncc(OC4CCNCC4)c3c2)s1] | 5-(4-(piperidin-4- yoxy)isoquinolin- 6-yl)-2- ((tetrahydro-2H- pyran-4- yl)ethynyl)thiazole | CD₃OD; δ 1.77-1.86 (2H, m), 1.97- 2.04 (2H, m), 2.26-2.44 (4H, m), 3.04-3.11 (1H, m), 3.34-3.40 (2H, m), 3.66-3.53 (4H, m), 3.92-4.00 (2H, m), 5.11-5.19 (1H, m), 8.19- 8.26 (1H, m), 8.32-8.40 (2H, m), 8.46 (1H, s), 8.54 (1H, s), 9.18 (1H, s). |

### Example 83

### 1-methylcyclopropyl (6-(1-methyl-1H-pyrazol-4-yl)isoquinolin-3-yl)carbamate

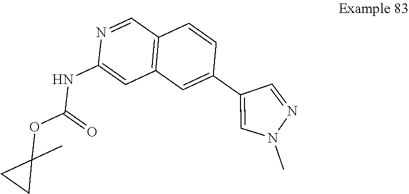 [SMILES: Cn1cc(-c2ccc3cnc(NC(=O)OC4(C)CC4)cc3c2)cn1]

### Step 1. Synthesis of phenyl (6-(1-methyl-1H-pyrazol-4-yl)isoquinolin-3-yl)carbamate

To a mixture of 6-(1-methyl-1H-pyrazol-4-yl)isoquinolin-3-amine (150 mg, 0.670 mmol) and pyridine (53 mg, 0.67 mmol) in DCM (6 mL) was added a solution of phenyl carbonochloridate (1.05 g, 6.69 mmol) in DCM (4 mL) at 0° C., then the mixture was stirred at 20° C. for 18 hours under N₂ atmosphere. The reaction mixture was quenched with water (15 mL) and extracted with EtOAc (15 mL×3). The combined organic layer was dried over anhydrous Na₂SO₄, filtered and concentrated. The residue was purified by flash silica gel column (PE/EtOAc=1/3) to give phenyl (6-(1-methyl-1H-pyrazol-4-yl)isoquinolin-3-yl)carbamate (210 mg, yield: 47%) as an off-white solid.

### Step 2. Synthesis of 1-methylcyclopropyl (6-(1-methyl-1H-pyrazol-4-yl)isoquinolin-3-yl)carbamate

To a solution of 1-methylcyclopropanol (84 mg, 1.2 mmol) and 15-crown-5 (26 mg, 0.12 mmol) in THF (2 mL) was added NaH (70 mg, 1.7 mmol, 60% dispersion in mineral oil) at 0° C. and stirred for 1 hour. A solution of phenyl (6-(1-methyl-1H-pyrazol-4-yl)isoquinolin-3-yl)carbamate (200 mg, 0.580 mmol) in THF (10 mL) was added and the resulting reaction mixture was stirred at 20° C. for another 4 hours under N₂ atmosphere. The reaction mixture was quenched with water (15 mL) and extracted with EtOAc (15 mL×3). The combined organic layer was dried over anhydrous Na₂SO₄, filtered and concentrated. The residue was purified by prep-HPLC (10 mM NH₄HCO₃ as an additive), then lyophilized to afford the title compound (8.57 mg, yield: 5%) as an off-white solid. ¹H NMR (400 MHz, DMSO-d₆) δ 0.68-0.74 (2H, m), 0.87-0.93 (2H, m), 1.58 (3H, s), 3.91 (3H, s), 7.73-7.75 (1H, m), 7.97-8.04 (2H, m), 8.08 (1H, s), 8.12 (1H, s), 8.34 (1H, s), 8.98 (1H, s), 10.01 (1H, brs).

The following compound was synthesized analogously to Example 83

| Example | | | |
|---|---|---|---|
| No. | Structure | Name | ¹H NMR (400 MHz) |
| 84 | 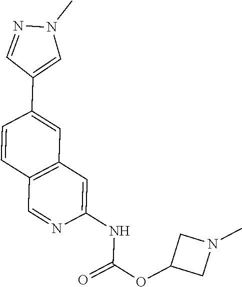 [SMILES: CN1CC(OC(=O)Nc2cc3cc(-c4cnn(C)c4)ccc3cn2)C1] | 1-methylazetidin-3- yl (6-(1-methyl-1H- pyrazol-4- yl)isoquinolin-3- yl)carbamate | DMSO-d₆; δ 2.29 (3H, s), 2.98- 3.04 (2H, m), 3.60-3.68 (2H, m), 3.91 (3H, s), 4.95-5.00 (1H, m), 7.74-7.80 (1H, m), 7.98-8.05 (2H, m), 8.08-8.10 (2H, m), 8.35 (1H, s), 9.00 (1H, s), 10.29 (1H, brs). |

### Example 85

### 4-((1-(methylsulfonyl)piperidin-4-yl)oxy)-6-(6-(trifluoromethyl)pyrazolo[1,5-a]pyridin-3-yl)isoquinoline

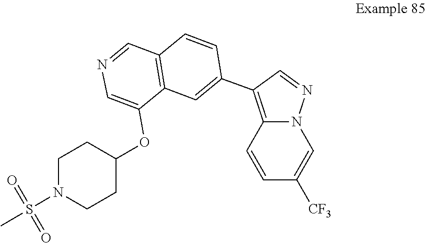 [SMILES: CS(=O)(=O)N1CCC(Oc2cncc3ccc(-c4cnn5cc(C(F)(F)F)ccc45)cc23)CC1]

### Step 1. Synthesis of tert-butyl 4-((6-(6-(trifluoromethyl)pyrazolo[1,5-a]pyridin-3-yl)isoquinolin-4-yl)oxy)piperidine-1-carboxylate

A mixture of compound Int-27 (792 mg, 1.74 mmol), compound Int-20 (120 mg, 0.380 mmol), XPhos-Pd-G₃ (32 mg, 0.038 mmol) and K₂CO₃ (106 mg, 0.770 mmol) in dioxane (10 mL) and H₂O (1.5 mL) was degassed and purged with N₂ for 3 times, then the mixture was stirred at 90° C. for 16 hours under N₂ atmosphere. The reaction mixture was concentrated and diluted with water (25 mL), then extracted with EtOAc (25 mL×3). The combined organic layer was dried over anhydrous Na₂SO₄, filtered and concentrated. The residue was purified by flash silica gel column (PE/EtOAc=1/3) to give tert-butyl 4-((6-(6-(trifluoromethyl)pyrazolo[1,5-a]pyridin-3-yl)isoquinolin-4-yl)oxy)piperidine-1-carboxylate (140 mg, yield: 31%) as yellow oil.

### Step 2. Synthesis of 4-(piperidin-4-yloxy)-6-(6-(trifluoromethyl)pyrazolo[1,5-a]pyridin-3-yl)isoquinoline

A solution of tert-butyl 4-((6-(6-(trifluoromethyl)pyrazolo[1,5-a]pyridin-3-yl)isoquinolin-4-yl)oxy)piperidine-1-carboxylate (135 mg, 0.260 mmol) in 4N HCl/EtOAc (5 mL) was stirred at 20° C. for 2 hours. The reaction mixture was concentrated to give 4-(piperidin-4-yloxy)-6-(6-(trifluoromethyl)pyrazolo[1,5-a]pyridin-3-yl)isoquinoline (104 mg, yield: 48%, HCl salt) as a yellow solid.

### Step 3. Synthesis of 4-((1-(methylsulfonyl)piperidin-4-yl)oxy)-6-(6-(trifluoromethyl)pyrazolo[1,5-a]pyridin-3-yl)isoquinoline

To a mixture of 4-(piperidin-4-yloxy)-6-(6-(trifluoromethyl)pyrazolo[1,5-a]pyridin-3-yl)isoquinoline (270 mg, 0.602 mmol, HCl salt) and Et₃N (609 mg, 6.02 mmol) in DCM (3 mL) was added a solution of MsCl (137 mg, 1.20 mmol) in DCM (1 mL) at 0° C. and then stirred at 20° C. for 4 hours under N₂ atmosphere. The reaction mixture was quenched with saturated aqueous NaHCO₃ (20 mL) and extracted with DCM (15 mL×3). The combined organic layer was dried over anhydrous Na₂SO₄, filtered and concentrated. The residue was purified by prep-HPLC (0.225% FA as an additive; Method C), then lyophilized to afford the title compound (21.17 mg, yield: 7%) as a light yellow solid. ¹H NMR (400 MHz, DMSO-d₆) δ 1.96-2.03 (2H, m), 2.11-2.18 (2H, m), 2.94 (3H, s), 3.23-3.27 (2H, m), 3.40-3.50 (2H, m), 4.88-4.90 (1H, m), 7.63-7.68 (1H, m), 8.05-8.17 (1H, m), 8.21-8.24 (2H, m), 8.32 (1H, s), 8.38 (1H, s), 8.84 (1H, s), 8.98 (1H, s), 9.48 (1H, s).

### Example 88

### 1-((6-(6-(trifluoromethyl)pyrazolo[1,5-a]pyridin-3-yl)isoquinolin-4-yl)methyl)piperidine-4-carboxylic acid

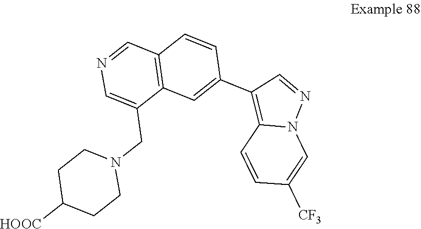 [SMILES: O=C(O)C1CCN(Cc2cncc3ccc(-c4cnn5cc(C(F)(F)F)ccc45)cc23)CC1]

To a solution of Example 87 (60 mg, 0.12 mmol) in THF (2 mL) and H₂O (0.5 mL) was added LiOH·H₂O (21 mg, 0.50 mmol). The mixture was stirred at 20° C. for 16 hours. The reaction mixture was acidified with 1N aqueous HCl to pH=5 and purified by prep-HPLC (0.225 FA as an additive) to give the title compound (6.5 mg, yield: 11%) as a yellow solid.

¹H NMR (400 MHz, DMSO-d₆) δ 1.53-1.70 (2H, m), 1.78-1.91 (2H, m), 2.05-2.14 (2H, m), 2.81-2.88 (2H, m), 3.33-3.37 (1H, m), 3.90 (2H, s), 7.65 (1H, dd, J=9.2, 1.6 Hz), 8.07 (1H, dd, J=8.8, 1.6 Hz), 8.23 (1H, d, J=8.4 Hz), 8.34 (1H, d, J=9.6 Hz), 8.39 (1H, s), 8.71 (1H, s), 8.84 (1H, s), 9.24 (1H, s), 9.50 (1H, s), 12.19 (1H, brs).

### Example 89

### 1-(4-((6-(6-(trifluoromethyl)pyrazolo[1,5-a]pyridin-3-yl)isoquinolin-4-yl)methyl)piperazin-1-yl)ethan-1-one

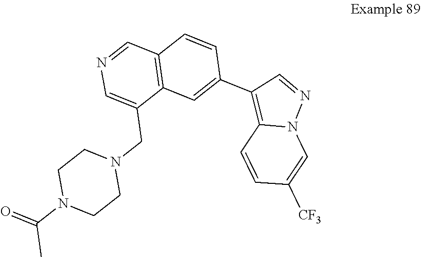 [SMILES: CC(=O)N1CCN(Cc2cncc3ccc(-c4cnn5cc(C(F)(F)F)ccc45)cc23)CC1]

### Step 1. Synthesis of 1-(4-((6-bromoisoquinolin-4-yl)methyl)piperazin-1-yl)ethan-1-one

To a solution of compound Int-28 (500 mg, 2.12 mmol) and 1-acetylpiperazine (543 mg, 4.24 mmol) in DCE (10 mL) was added HOAc (127 mg, 2.12 mmol) at 25° C., then NaBH(OAc)₃ (1.35 g, 6.35 mmol) was added to this solution. The reaction mixture was stirred at 25° C. for 2 hours. The reaction mixture was quenched by addition water (20 mL) and extracted with DCM (20 mL×3). The combined organic layer was washed with brine (20 mL), dried over anhydrous Na₂SO₄, filtered and concentrated. The residue was purified by Combi Flash (0% to 100% EtOAc in PE) to give 1-(4-((6-bromoisoquinolin-4-yl)methyl)piperazin-1-yl)ethan-1-one (700 mg, yield: 92%) as a light yellow solid.

### Step 2. Synthesis of 1-(4-((6-(6-(trifluoromethyl)pyrazolo[1,5-a]pyridin-3-yl)isoquinolin-4-yl)methyl)piperazin-1-yl)ethan-1-one

A mixture of 1-(4-((6-bromoisoquinolin-4-yl)methyl)piperazin-1-yl)ethan-1-one (120 mg, 0.304 mmol), Bis-Pin (98 mg, 0.384 mmol), Pd(dppf)Cl₂ (28 mg, 0.0384 mmol) and KOAc (75 mg, 0.768 mmol) in anhydrous dioxane (3 mL) and H₂O (0.5 mL) was degassed and purged with N₂ for 3 times, the mixture was stirred at 90° C. for 1 hour under N₂ atmosphere. The reaction mixture was cooled to room temperature, dioxane (3 mL) and H₂O (0.5 mL) were added to the reaction mixture. Then compound Int-20 (95 mg, 0.30 mmol), Pd(dppf)Cl₂ (22 mg, 0.030 mmol) and Na₂CO₃ (64 mg, 061 mmol) were added and the reaction was degassed and purged with N₂ for 3 times. The reaction mixture was stirred at 90° C. for another 2 hours under N₂ atmosphere.

The reaction mixture was filtered through a pad of celite and the filtrate was concentrated and the residue was purified by pre-HPLC (0.225% FA as an additive; Method C) to give the title compound (69.28 mg, yield: 50%) as a yellow solid. ¹H NMR (400 MHz, DMSO-d₆) δ 1.99 (3H, s), 2.40-2.44 (2H, m), 2.45-2.48 (2H, m), 3.40-3.51 (4H, m), 3.95 (2H, s), 7.67-7.74 (1H, m), 8.09 (1H, dd, J=8.4, 1.2 Hz), 8.24 (1H, d, J=8.4 Hz), 8.30 (1H, d, J=9.2 Hz), 8.41 (1H, s), 8.62 (1H, s), 8.85 (1H, s), 9.25 (1H, s), 9.50 (1H, s).

The following compounds were synthesized analogously to Example 89

| Example | | | |
|---|---|---|---|
| No. | Structure | Name | ¹H NMR (400 MHz) |
| 101 | 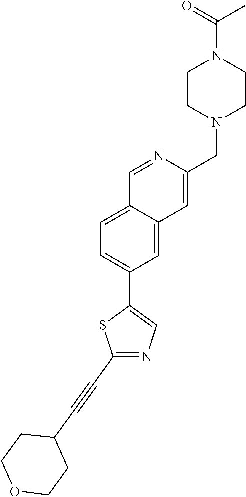 [SMILES: CC(=O)N1CCN(Cc2cc3cc(-c4cnc(C#CC5CCOCC5)s4)ccc3cn2)CC1] | 1-(4-((6-(2- ((tetrahydro-2H- pyran-4- yl)ethynyl)thiazol-5- yl)isoquinolin-3- yl)methyl)piperazin- 1-yl)ethan-1-one | CD₃OD; δ 1.70-1.86 (2H, m), 1.89-2.05 (2H, m), 2.10 (3H, s), 2.54-2.63 (4H, m), 2.87-3.13 (1H, m), 3.55-3.66 (6H, m), 3.84 (2H, s), 3.88-4.00 (2H, m), 7.92 (1H, s), 7.97 (1H, d, J = 8.4 Hz), 8.11- 8.22 (2H, m), 8.31 (1H, s), 9.23 (1H, s). |
| 102 | 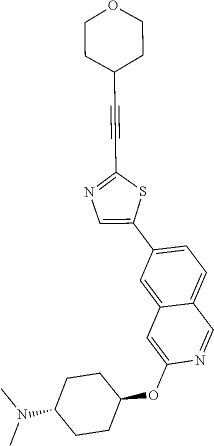 [SMILES: CN(C)[C@H]1CC[C@H](Oc2cc3cc(-c4cnc(C#CC5CCOCC5)s4)ccc3cn2)CC1] | (1r,4r)-N,N-dimethyl- 4-((6-(2-((tetrahydro- 2H-pyran-4- yl)ethynyl)thiazol-5- yl)isoquinolin-3- yl)oxy)cyclohexan-1- amine | CD₃OD; δ 1.72-1.87 (8H, m), 1.93-2.02 (2H, m), 2.19-2.28 (2H, m), 2.49 (6H, s), 2.62-2.72 (1H, m), 2.98-3.09 (1H, m), 3.54-3.62 (2H, m), 3.90-3.98 (2H, m), 5.16-5.23 (1H, m), 7.19 (1H, s), 7.72 (1H, dd, J = 8.4, 1.2 Hz), 7.99-8.07 (2H, m), 8.27 (1H, s), 8.96 (1H, s). |
| 103 | 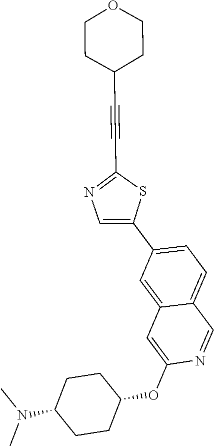 [SMILES: CN(C)[C@H]1CC[C@@H](Oc2cc3cc(-c4cnc(C#CC5CCOCC5)s4)ccc3cn2)CC1] | (1s,4s)-N,N- dimethyl-4-((6-(2- ((tetrahydro-2H- pyran-4- yl)ethynyl)thiazol-5- yl)isoquinolin-3- yl)oxy)cyclohexan-1- amine | CD₃OD; δ 1.43-1.62 (4H, m), 1.73-1.84 (2H, m), 1.93-2.09 (4H, m), 2.26-2.33 (2H, m), 2.35 (6H, s), 2.38-2.44 (1H, m), 2.99-3.07 (1H, m), 3.54-3.63 (2H, m), 3.90-3.98 (2H, m), 4.80-4.85 (1H, m), 7.12 (1H, s), 7.67-7.73 (1H, m), 7.98-8.06 (2H, m), 8.26 (1H, s), 8.93 (1H, s). |
| 104 | 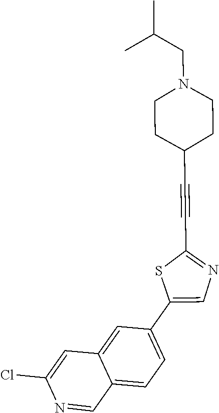 [SMILES: CC(C)CN1CCC(C#Cc2ncc(-c3ccc4cnc(Cl)cc4c3)s2)CC1] | 5-(3- chlorosiqouinolin-6- yl)-2-((1- isobutylpiperidin-4- yl)ethynyl)thiazole | CD₃OD; δ 0.99 (6H, d, J = 6.4 Hz), 1.87-2.17 (5H, m), 2.26-2.81 (4H, m), 2.86-3.19 (3H, m), 7.94 (1H, s), 7.99 (1H, dd, J = 8.4, 1.6 Hz), 8.15-8.20 (2H, m), 8.34 (1H, s), 9.10 (1H, s). |
| 105 | 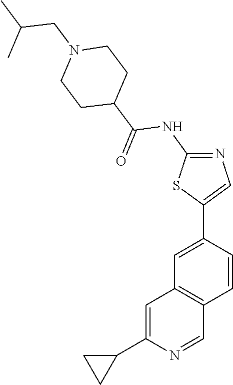 [SMILES: CC(C)CN1CCC(C(=O)Nc2ncc(-c3ccc4cnc(C5CC5)cc4c3)s2)CC1] | N-(5-(3- cyclopropylisoquinolin- 6-yl)thiazol-2-yl)- 1-isobutylpiperidine- 4-carboxamide | DMSO-d₆; δ 0.86 (6H, d, J = 6.4 Hz), 0.95-1.04 (4H, m), 1.60-1.85 (5H, m), 1.85-1.96 (2H, m), 2.05 (2H, d, J = 7.2 Hz), 2.16-2.27 (1H, m), 2.43-2.49 (1H, m), 2.85-2.90 (2H, m), 7.69 (1H, s), 7.88 (1 H, dd, J = 8.4, 1.6 Hz), 7.98 (1H, s), 8.04 (1H, d, J = 8.8 Hz), 8.11 (1H, s), 9.12 (1H, s), 12.28 (1H, brs). |

### Example 90

### ethyl 1-((6-(2-((tetrahydro-2H-pyran-4-yl)ethynyl)thiazol-5-yl)isoquinolin-4-yl)methyl)piperidine-4-carboxylate

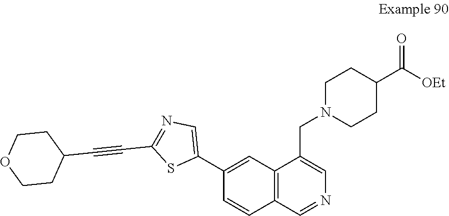 [SMILES: CCOC(=O)C1CCN(Cc2cncc3ccc(-c4cnc(C#CC5CCOCC5)s4)cc23)CC1]

A mixture of ethyl 1-((6-bromoisoquinolin-4-yl)methyl)piperidine-4-carboxylate (300 mg, 0.795 mmol), 2-((tetrahydro-2H-pyran-4-yl)ethynyl)thiazole (231 mg, 1.19 mmol), Pd(OAc)₂ (18 mg, 0.08 mmol), t-Bu₃PHBF₄ (46 mg, 0.16 mmol) and Cs₂CO₃ (518 mg, 1.59 mmol) in DMF (10 mL) was degassed and purged with N₂ for 3 times, then the mixture was stirred at 120° C. for 2 hours under N₂ atmosphere. The reaction mixture was diluted with H₂O (25 mL) and extracted EtOAc (50 mL×3). The combined organic layers were dried over anhydrous Na₂SO₄, filtered and concentrated. The residue was purified by Combi Flash (0% to 30% EtOAc in PE) to give the title compound (140 mg, yield: 36%) as a white solid.

### Example 92

### 5-((5-(isoquinolin-6-yl)thiazol-2-yl)ethynyl)-1-methylpyridin-2(1H)-one

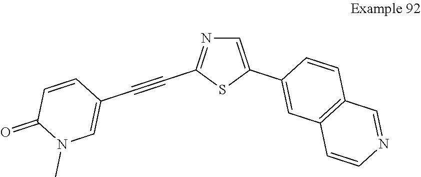 [SMILES: Cn1cc(C#Cc2ncc(-c3ccc4cnccc4c3)s2)ccc1=O]

### Step 1. Synthesis of 5-((5-bromothiazol-2-yl)ethynyl)-1-methylpyridin-2(1H)-one

A mixture of compound Int-29 (500 mg, 3.76 mmol), 5-bromo-2-iodothiazole (1.31 g, 4.51 mmol), Pd(PPh₃)₂Cl₂ (264 mg, 0.38 mmol), CuI (143 mg, 0.751 mmol) and Et₃N (1.90 g, 18.8 mmol) in THF (5 mL) was degassed and purged with N₂ for 3 times, and then the mixture was stirred at 20° C. for 16 hours under N₂ atmosphere. The reaction mixture was filtered and the filtrate was concentrated. The residue was purified by flash silica gel chromatography (DCM/MeOH=10/1), then triturated with CH₃CN (4 mL) to afford 5-((5-bromothiazol-2-yl)ethynyl)-1-methylpyridin-2(1H)-one (370 mg, yield: 33%) as a yellow solid. ¹H NMR (400 MHz, DMSO-d₆) δ 3.45 (3H, s), 6.44 (1H, d, J=9.2 Hz), 7.60 (1H, dd, J=9.2, 2.4 Hz), 8.02 (1H, s), 8.36 (1H, d, J=2.4 Hz).

### Step 2. Synthesis of 5-((5-(isoquinolin-6-yl)thiazol-2-yl)ethynyl)-1-methylpyridin-2(1H)-one

A mixture of 5-((5-bromothiazol-2-yl)ethynyl)-1-methylpyridin-2(1H)-one (270 mg, 0.914 mmol), compound Int-1 (466 mg, 1.83 mmol), Pd(dtbpf)Cl₂ (59 mg, 0.91 mmol) and Na₂CO₃ (290 mg, 2.74 mmol) in 1, 4-dioxane (4 mL) and H₂O (0.4 mL) was degassed and purged with N₂ for 3 times, and then the mixture was stirred at 90° C. for 1 hour under N₂ atmosphere. The reaction mixture was concentrated and the residue was purified by silica gel column (DCM/MeOH=10/1), then further purified by prep-HPLC (0.225% FA as an additive; Method C), then lyophilized to afford the title compound (90 mg, yield: 22%, FA salt) as a yellow solid. ¹H NMR (400 MHz, DMSO-d₆) δ 3.47 (3H, s), 6.46 (1H, d, J=9.6 Hz), 7.62 (1H, dd, J=9.2, 2.4 Hz), 7.86 (1H, d, J=6.0 Hz), 8.06 (1H, dd, J=8.4, 1.2 Hz), 8.21 (1H, d, J=8.4 Hz), 8.28 (1H, s), 8.38 (1H, d, J=2.4 Hz), 8.54 (1H, d, J=5.6 Hz), 8.57 (1H, s), 9.32 (1H, s).

### Example 106

### 6-(1-methyl-1H-pyrazol-4-yl)-3-(piperidin-4-ylethynyl)isoquinoline

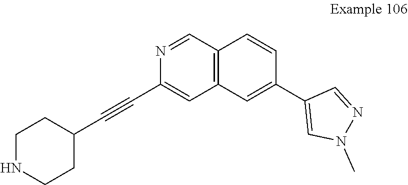 [SMILES: Cn1cc(-c2ccc3cnc(C#CC4CCNCC4)cc3c2)cn1]

### Step 1. Synthesis of tert-butyl 4-((6-(1-methyl-1H-pyrazol-4-yl)isoquinolin-3-yl)ethynyl)piperidine-1-carboxylate

A mixture of compound Int-36 (200 mg, 8.20 mmol), tert-butyl 4-ethynylpiperidine-1-carboxylate (206 mg, 9.85 mmol), Pd(CH₃CN)₂Cl₂ (21 mg, 0.82 mmol), XPhos (78 mg, 0.16 mmol) and Cs₂CO₃ (802 mg, 2.50 mmol) in CH₃CN (5 mL) was degassed and purged with N₂ for 3 times, then the mixture was stirred at 80° C. for 16 hours under N₂ atmosphere. The reaction mixture was concentrated and the residue was purified by silica gel column (PE/EtOAc=1/1) to afford tert-butyl 4-((6-(1-methyl-1H-pyrazol-4-yl)isoquinolin-3-yl)ethynyl)piperidine-1-carboxylate (200 mg, yield: 56%) as yellow gum. ¹H NMR (400 MHz, DMSO-d₆) δ 1.41 (9H, s), 1.53-1.59 (2H, m), 1.84-1.90 (2H, m), 2.89-2.96 (1H, m), 3.11-3.17 (2H, m), 3.66-3.72 (2H, m), 3.90 (3H, s), 7.86 (1H, s), 7.91 (1H, dd, J=8.4, 1.6 Hz), 8.04 (1H, s), 8.05-8.10 (2H, m), 8.33 (1H, s), 9.15 (1H, s).

### Step 2. Synthesis of 6-(1-methyl-1H-pyrazol-4-yl)-3-(piperidin-4-ylethynyl)isoquinoline

To a solution of tert-butyl 4-((6-(1-methyl-1H-pyrazol-4-yl)isoquinolin-3-yl)ethynyl)piperidine-1-carboxylate (30 mg, 0.072 mmol) in DCM (2 mL) was added TFA (0.5 mL). The mixture was stirred at 20° C. for 1 hour. The reaction mixture was concentrated and the residue was purified by prep-HPLC (0.225% FA as an additive; Method C), then lyophilized to afford the title compound (9.01 mg, yield: 39%, FA salt) as a yellow solid. ¹H NMR (400 MHz, DMSO-d₆) δ 1.68-1.79 (2H, m), 1.95-2.03 (2H, m), 2.81-2.88 (2H, m), 2.91-2.99 (1H, m), 3.09-3.17 (2H, m), 3.91 (3H, s), 7.87 (1H, s), 7.92 (1H, dd, J=8.8, 1.6 Hz), 8.05 (1H, s), 8.06-8.13 (2H, m), 8.34 (1H, s), 8.38 (1H, s), 9.16 (1H, s).

### Example 108

### 6-(1-methyl-1H-pyrazol-4-yl)-3-((tetrahydro-2H-pyran-4-yl)ethynyl)isoquinoline

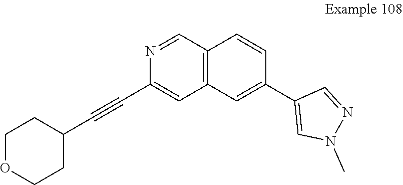 [SMILES: Cn1cc(-c2ccc3cnc(C#CC4CCOCC4)cc3c2)cn1]

A mixture of compound Int-36 (60 mg, 0.25 mmol), Pd(CH₃CN)₂Cl₂ (6 mg, 0.02 mmol), X-Phos (23 mg, 0.049 mmol) and Cs₂CO₃ (241 mg, 0.739 mmol) in CH₃CN (3 mL) was degassed and purged with N₂ for 3 times, 4-ethynyltetrahydro-2H-pyran (136 mg, 1.23 mmol) was added and the reaction mixture was stirred at 80° C. for 16 hours under N₂ atmosphere. The reaction mixture was filtered and the filtrate was concentrated. The residue was purified by prep-HPLC (0.05% NH₃·H₂O as an additive; Method B) and lyophilized to afford the title compound (25.65 mg, yield: 20%) as a yellow solid. ¹H NMR (400 MHz, DMSO-d₆) δ 1.61-1.71 (2H, m), 1.85-1.92 (2H, m), 2.93-3.00 (1H, m), 3.44-3.51 (2H, m), 3.81-3.87 (2H, m), 3.91 (3H, s), 7.86 (1H, s), 7.92 (1H, dd, J=8.8, 2.0 Hz), 8.05 (1H, s), 8.07-8.11 (2H, m), 8.34 (1H, s), 9.16 (1H, s).

### Example 110

### N-(5-(isoquinolin-6-yl)thiazol-2-yl)-1-methyl-6-oxo-1,6-dihydropyridine-3-carboxamide

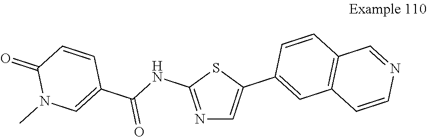 [SMILES: Cn1cc(C(=O)Nc2ncc(-c3ccc4cnccc4c3)s2)ccc1=O]

### Step 1: Synthesis of (E)-6-(2-ethoxyvinyl)isoquinoline

To a microwave vial (20 mL) was added 6-bromoisoquinoline (1.15 g, 5.53 mmol), (E)-2-(2-ethoxyvinyl)-4,4,5,5-tetramethyl-1,3,2-dioxaborolane (1.31 g, 6.63 mmol), Na₂CO₃ (1.79 g, 16.6 mmol), Pd(dppf)Cl₂ (405 mg, 0.55 mmol) followed by 1,4-dioxane (8 mL) and water (2 mL). The mixture was purged with nitrogen 3 times. The reaction was then heated to 90° C. After 16 h, the reaction was cooled to rt, diluted with EtOAc, and filtered through a pad of Celite. The filtrate was concentrated under reduced pressure to give the crude. The crude was purified by flash silica gel chromatography (0-40% EtOAc/hexanes) to give (E)-6-(2-ethoxyvinyl)isoquinoline (1.09 g, 99%). ¹H NMR (400 MHz, DMSO-d₆) δ 1.29 (t, J=6.8 Hz, 3H), 3.98 (q, J=6.8 Hz, 2H), 6.04 (d, J=12.8 Hz, 1H), 7.53 (d, J=12.8 Hz, 1H), 7.65 (d, J=6.0 Hz, 1H), 7.69 (s, 1H), 7.74 (dd, J=8.8, 1.6 Hz, 1H), 7.97 (d, J=8.8 Hz, 1H), 8.40 (d, J=6.0 Hz, 1H), 9.16 (s, 1H); LCMS: RT=0.81 min, ES-MS [M+H]⁺=200.2.

### Step 2. Synthesis of 5-(isoquinolin-6-yl)thiazol-2-amine

To a solution of (E)-6-(2-ethoxyvinyl)isoquinoline (1.62 g, 8.13 mmol) in a mixture of 1,4-dioxane (20 mL) and water (20 mL) was added N-Bromosuccinimide (1.59 g, 68.94 mmol) at 0° C. After stirring 30 min at 0° C., thiourea (680.8 mg, 8.94 mmol) was added. The reaction was then heated to 100° C. After 1 h, the reaction was cooled to RT, concentrated under reduced pressure to give the crude. The crude was purified by flash silica gel chromatography (0-15% MeOH/DCM) to give 5-(isoquinolin-6-yl)thiazol-2-amine (1.24 g, 67%). ¹H NMR (400 MHz, DMSO-d₆) δ 7.67 (brs, 2H), 7.81-7.91 (m, 2H), 8.00 (d, J=6.0 Hz, 1H), 8.07 (dd, J=8.8, 1.6 Hz, 1H), 8.19 (d, J=8.8 Hz, 1H), 8.49 (d, J=6.0 Hz, 1H), 9.39 (s, 1H); LCMS: RT=0.56 min, ES-MS [M+H]⁺=228.2.

### Step 3. Synthesis of N-(5-(isoquinolin-6-yl)thiazol-2-yl)-1-methyl-6-oxo-1,6-dihydropyridine-3-carboxamide

To a mixture of 5-(isoquinolin-6-yl)thiazol-2-amine (15 mg, 0.07 mmol), 1-methyl-6-oxo-1,6-dihydropyridine-3-carboxylic acid (15 mg, 1.0 mmol), EDCI (25 mg, 0.13 mmol), HOBt (20 mg, 0.13 mmol), DMAP (8 mg, 0.07 mmol) and N,N-diisopropylethylamine (34 μL, 0.20 mmol) was added DMF (1 mL). The reaction was then heated to 90° C. Upon completion, the reaction was diluted with DMSO (1 mL), filtered through a syringe filter to give the crude. Crude product was purified using prep HPLC (5-95% ACN/0.1% aqueous TFA over 10 min). Fractions containing desired product were basified with sat. NaHCO₃ then extracted with 3:1 chloroform/IPA (3×). The combined organics were passed through a phase separator and the solvents were concentrated to give N-(5-(isoquinolin-6-yl)thiazol-2-yl)-1-methyl-6-oxo-1,6-dihydropyridine-3-carboxamide (10.7 mg, 45%). ¹H NMR (400 MHz, DMSO) δ 9.27 (s, 1H), 8.75 (d, J=2.7 Hz, 1H), 8.50 (d, J=5.7 Hz, 1H), 8.22 (s, 1H), 8.18-8.11 (m, 2H), 8.10-8.02 (m, 2H), 7.88-7.82 (m, 1H), 6.48 (d, J=9.5 Hz, 1H), 3.53 (s, 3H); ES-MS [M+H]⁺=363.0.

The following compounds were synthesized analogously to Example 110

| Example | | | |
|---|---|---|---|
| No. | Structure | Name | ¹H NMR (400 MHz); ES-MS |
| 111 | 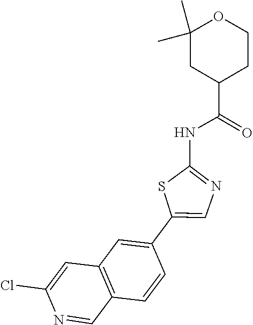 [SMILES: CC1(C)CC(C(=O)Nc2ncc(-c3ccc4cnc(Cl)cc4c3)s2)CCO1] | N-(5-(3- chloroisoquinolin-6- yl)thiazol-2-yl)-2,2- dimethyltetrahydro- 2H-pyran-4- carboxamide | DMSO; δ 9.15 (s, 1H), 8.20 - 8.16 (m, 2H), 8.10 (d, J = 1.7 Hz, 1H), 8.06 (dd, J = 8.6, 1.8 Hz, 1H), 8.02 (s, 1H), 3.68 (ddd, J= 11.9, 5.2, 1.7 Hz, 1H), 3.58 (td, J= 12.1, 2.4 Hz, 1H), 2.98 (tt, J = 12.3, 3.8 Hz, 1H), 1.77 - 1.65 (m, 2H), 1.57 (qd, J = 12.4, 5.1 Hz, 1H), 1.45 (t, J = 12.7 Hz, 1H), 1.19 (s, 3H), 1.16 (s, 3H); ES-MS [M + H]⁺ = 402.1 |
| 112 | 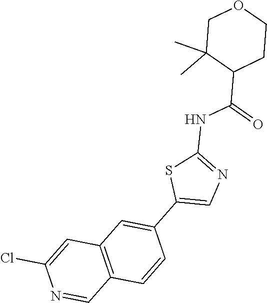 [SMILES: CC1(C)COCCC1C(=O)Nc1ncc(-c2ccc3cnc(Cl)cc3c2)s1] | N-(5-(3- chloroisoquinolin-6- yl)thiazol-2-yl)-3,3- dimethyltetrahydro- 2H-pyran-4- carboxamide | ES-MS [M + H]⁺ = 402.3 |
| 113 | 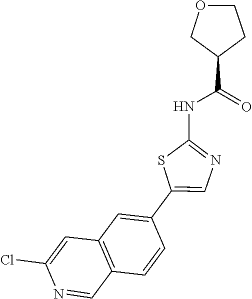 [SMILES: O=C(Nc1ncc(-c2ccc3cnc(Cl)cc3c2)s1)[C@@H]1CCOC1] | (R)-N-(5-(3- chloroisoquinolin-6- yl)thiazol-2- yl)tetrahydrofuran-3- carboxamide | DMSO; δ 9.15 (s, 1H), 8.21 - 8.15 (m, 2H), 8.10 (d, J = 1.7 Hz, 1H), 8.07 (dd, J = 8.6, 1.8 Hz, 1H), 8.02 (s, 1H), 3.94 (t, J = 8.3 Hz, 1H), 3.86-3.75 (m, 2H), 3.72 (dt, J= 8.3, 7.0 Hz, 1H), 3.36 - 3.32 (m, 1H), 2.21 - 2.04 (m, 2H); ES-MS [M + H]⁺ = 360.1 |
| 114 | 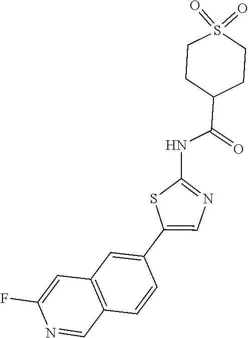 [SMILES: O=C(Nc1ncc(-c2ccc3cnc(F)cc3c2)s1)C1CCS(=O)(=O)CC1] | N-(5-(3- fluoroisoquinolin-6- yl)thiazol-2- yl)tetrahydro-2H- thiopyran-4- carboxamide 1,1- dioxide | ES-MS [M + H]⁺ = 406.0 |
| 115 | 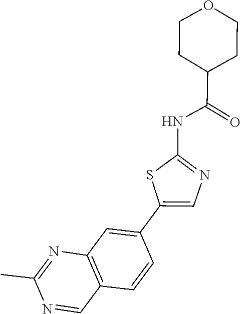 | N-(5-(2- methylquinazolin-7- yl)thiazol-2- yl)tetrahydro-2H- pyran-4-carboxamide | DMSO; δ 9.44 (s, 1H), 8.24 (s, 1H), 8.12 (d, J = 9.2 Hz, 1H), 8.01-7.97 (m, 2H), 3.95-3.86 (m, 2H), 3.40-3.34 (m, 2H), 2.80 (td, J = 7.2, 3.7 Hz, 1H), 2.76 (s, 3H), 1.80-1.63 (m, 4H); ES-MS [M + H₂O + H]⁺ = 373.0 |
| 116 | 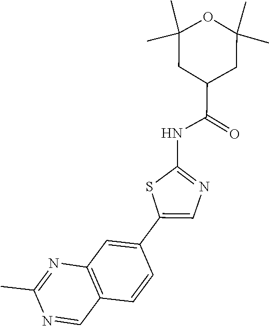 | 2,2,6,6-tetramethyl- N-(5-(2- methylquinazolin-7- yl)thiazol-2- yl)tetrahydro-2H- pyran-4-carboxamide | DMSO; δ 9.45 (d, J = 0.7 Hz, 1H), 8.25 (s, 1H), 8.12 (dd, J = 8.4, 0.8 Hz, 1H), 8.03-7.95 (m, 2H), 3.19-3.08 (m, 1H), 2.76 (s, 3H), 1.75 (dd, J= 13.0, 3.2 Hz, 2H), 1.39 (t, J = 12.6 Hz, 2H), 1.24 (s, 6H), 1.15 (s, 6H); ES-MS [M + H₂O + H]⁺ = 429.1 |

### Intermediates of Formula (II)

### Intermediate 1

### 4-(2-aminothiazol-5-yl)benzonitrile

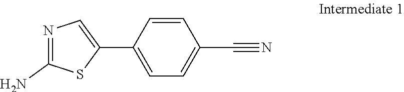 [SMILES: N#Cc1ccc(-c2cnc(N)s2)cc1]

To a solution of (E)-4-(2-ethoxyvinyl)benzonitrile (1.38 g, 7.97 mmol) in dioxane (10 mL) and H₂O (10 mL) was added NBS (1.56 g, 8.76 mmol) at 0° C. Then the mixture was stirred at 25° C. for 30 minutes. Thiourea (667 mg, 8.76 mmol) was added to the reaction mixture and the resulting reaction mixture was stirred at 100° C. for 1 hour. The reaction mixture was concentrated. The crude product was triturated with MeOH (10 mL) to give 4-(2-aminothiazol-5-yl)benzonitrile (1.00 g, yield: 62%) as a yellow solid.

¹H NMR (400 MHz, DMSO-d₆) δ 7.75 (2H, d, J=8.4 Hz), 7.88 (2H, d, J=8.4 Hz), 7.98 (1H, s), 9.10 (2H, brs).

### Intermediate 2

### tert-butyl 4-((4-bromopyridin-2-yl)carbamoyl)piperidine-1-carboxylate

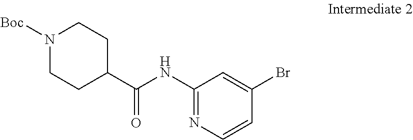 [SMILES: CC(C)(C)OC(=O)N1CCC(C(=O)Nc2cc(Br)ccn2)CC1]

To a solution 4-bromopyridin-2-amine (4.15 g, 24.0 mmol) and 1-(tert-butoxycarbonyl)piperidine-4-carboxylic acid (5.00 g, 21.8 mmol) in pyridine (40 mL) was added T₃P (27.8 g, 43.6 mmol, 50% purity in EtOAc) and Et₃N (6.62 g, 65.4 mmol) at 25° C. The mixture was stirred at 25° C. for 3 hours. The reaction mixture was concentrated and the residue was basified with saturated aqueous NaHCO₃ to pH=8, then extracted with EtOAc (100 mL×3). The combined organic layers were washed with brine (150 mL), dried over anhydrous Na₂SO₄, filtered and concentrated to give tert-butyl 4-((4-bromopyridin-2-yl)carbamoyl)piperidine-1-carboxylate (7.00 g, yield: 84%) as a yellow solid.

¹H NMR (400 MHz, CDCl₃) δ 1.47 (9H, s), 1.63-1.79 (2H, m), 1.82-1.95 (2H, m), 2.40-2.45 (1H, m), 2.75-2.85 (2H, m), 4.10-4.26 (2H, m), 7.21 (1H, dd, J=5.2, 1.6 Hz), 8.07 (1H, d, J=5.2 Hz), 8.19 (1H, brs), 8.49 (1H, d, J=1.6 Hz).

### Intermediate 3

### 5-bromo-3-methylbenzo[d]isothiazole

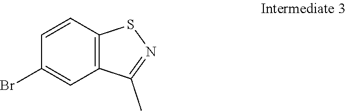

### Step 1. Synthesis of 3-methylbenzo[d]isothiazol-5-amine

To a solution of 3-methyl-5-nitrobenzo[d]isothiazole (900 mg, 4.63 mmol) in EtOH (20 mL) and H₂O (5 mL) was added Fe powder (1.29 g, 23.2 mmol) and NH₄Cl (2.48 g, 46.3 mmol) at 25° C. The mixture was stirred at 80° C. for 0.5 hour. The reaction mixture was filtered and the filtrate was concentrated. The residue was diluted with H₂O (25 mL) and extracted with EtOAc (25 mL×3). The combined organic layers were washed with brine (25 mL), dried over anhydrous Na₂SO₄, filtered and concentrated to give 3-methylbenzo[d]isothiazol-5-amine (0.72 g, yield: 95%) as a white solid.

### Step 2. Synthesis of 5-bromo-3-methylbenzo[d]isothiazole

To a solution of 3-methylbenzo[d]isothiazol-5-amine (700 mg, 4.26 mmol) in CH₃CN (10 mL) was added tert-butyl nitrite (879 mg, 8.52 mmol) and CuBr₂ (2.38 g, 10.7 mmol) at 25° C. The mixture was stirred at 80° C. for 0.5 hour. The reaction mixture was diluted with H₂O (40 mL) and extracted with EtOAc (40 mL×3). The combined organic layers were washed with brine (25 mL), dried over anhydrous Na₂SO₄, filtered and concentrated. The residue was purified by column chromatography (SiO₂, Petroleum ether/Ethyl acetate=1/0 to 10/1) to give 5-bromo-3-methylbenzo[d]isothiazole (500 mg, yield: 45%) as yellow gum.

### Intermediate 4

### 4-(3-amino-1H-pyrazol-5-yl)benzonitrile

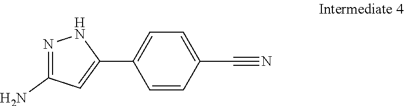 [SMILES: N#Cc1ccc(-c2cc(N)n[nH]2)cc1]

### Step 1. Synthesis of 4-(2-cyanoacetyl)benzonitrile

To a suspension of NaH (844 mg, 21.1 mmol, 60% dispersion in mineral oil) in anhydrous THE (20 mL) was added CH₃CN (611 mg, 14.9 mmol), the mixture was stirred at 20° C. for 0.25 hour. methyl 4-cyanobenzoate (2.00 g, 12.4 mmol) was added to the mixture and the reaction mixture was stirred at 60° C. for 3 hours. The mixture was carefully acidified with 1N aqueous HCl to pH=2 and diluted with H₂O (40 mL), then concentrated. The mixture was extracted with EtOAc (40 mL×3) and the combined organic layer was washed with brine (50 mL), dried anhydrous Na₂SO₄, filtered and concentrated to give 4-(2-cyanoacetyl)benzonitrile (1.80 g, crude) as a yellow solid, which was used for the next step without further purification.

¹H NMR (400 MHz, DMSO-d₆) δ 4.48-5.76 (2H, m), 7.73-8.34 (4H, m).

### Step 2. Synthesis of 4-(3-amino-1H-pyrazol-5-yl)benzonitrile

A solution of 4-(2-cyanoacetyl)benzonitrile (1.50 g, 8.81 mmol), HOAc (1.32 g, 22.0 mmol) and hydrazine hydrate (1.00 g, 20.0 mmol, 80% purity) in EtOH (20 mL) was stirred at 80° C. for 3 hours. The reaction mixture was concentrated and the residue was basified with saturated aqueous NaHCO₃ to pH=8 and diluted into H₂O (30 mL), then extracted with EtOAc (30 mL×2). The combined organic layer was washed with brine (50 mL), dried over anhydrous Na₂SO₄, filtered and concentrated. The residue was purified by flash silica gel chromatography (ISCO®; 12 g SepaFlash® Silica Flash Column, Eluent of 0-10% methanol/dichloromethane gradient @25 mL/min) to give 4-(3-amino-1H-pyrazol-5-yl)benzonitrile (1.00 g, yield: 62% for two steps) as a yellow solid.

¹H NMR (400 MHz, DMSO-d₆) δ 4.44-5.37 (2H, m), 5.62-6.19 (1H, m), 7.66-7.93 (4H, m), 11.63-12.35 (1H, m).

### Intermediate 5

### N-(5-bromothiazol-2-yl)-1-isobutyl-6-oxo-1,6-dihydropyridine-3-carboxamide

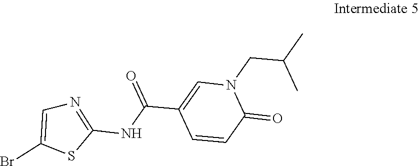 [SMILES: CC(C)Cn1cc(C(=O)Nc2ncc(Br)s2)ccc1=O]

### Step 1. Synthesis of 5-bromo-1-isobutylpyridin-2(1H)-one

A mixture of 5-bromopyridin-2(1H)-one (5.00 g, 28.7 mmol), 1-iodo-2-methyl-propane (15.9 g, 86.2 mmol) and Cs₂CO₃ (46.8 g, 144 mmol) in anhydrous DMF (100 mL) was stirred at 25° C. for 18 hours. The reaction mixture was diluted with water (500 mL) and extracted with EtOAc (500 mL×3). The combined organic layer was washed with brine (250 mL×3), dried over anhydrous Na₂SO₄, filtered and concentrated. The residue was purified by flash silica gel chromatography (ISCO®; 40 g SepaFlash® Silica Flash Column, Eluent of 0˜30% Ethyl acetate/Petroleum ether gradient @40 mL/min) to give 5-bromo-1-isobutylpyridin-2(1H)-one (2.60 g, yield: 36%) as a yellow oil.

### Step 2. Synthesis of methyl 1-isobutyl-6-oxo-1,6-dihydropyridine-3-carboxylate

A mixture of 5-bromo-1-isobutylpyridin-2(1H)-one (1.00 g, 4.35 mmol), Pd(dppf)Cl₂ (318 mg, 0.435 mmol) and K₂CO₃ (1.80 g, 13.0 mmol) in MeOH (10 mL) was degassed and purged with CO for 3 times. Then the mixture was stirred at 60° C. for 16 hour under CO (50 psi) atmosphere. The reaction mixture was filtered and the filtrate was concentrated. The residue was purified by flash silica gel chromatography (ISCO®; 12 g SepaFlash® Silica Flash Column, Eluent of 0˜40% Ethyl acetate/Petroleum ether gradient @40 mL/min) to give methyl 1-isobutyl-6-oxo-1,6-dihydropyridine-3-carboxylate (680 mg, yield: 45%) as colorless oil.

¹H NMR (400 MHz, CDCl₃) δ 0.98 (6H, d, J=6.8 Hz), 2.10-2.30 (1H, m), 3.80 (2H, d, J=7.2 Hz), 3.88 (3H, s), 7.30-7.40 (1H, m), 7.85 (1H, dd, J=9.2, 2.0 Hz), 8.12 (1H, d, J=2.4 Hz).

### Step 3. Synthesis of 1-isobutyl-6-oxo-1,6-dihydropyridine-3-carboxylic acid

A mixture of methyl 1-isobutyl-6-oxo-1,6-dihydropyridine-3-carboxylate (680 mg, 3.25 mmol), LiOH·H₂O (409 mg, 9.75 mmol) in THF (4 mL), MeOH (2 mL) and H₂O (1 mL) was stirred at 25° C. for 2 hours. The reaction mixture was diluted with water (25 mL) and washed with EtOAc (20 mL×3). The aqueous layer was acidified with 1N aqueous to pH=2 and extracted with EtOAc (20 mL×3). The combined organic layer was dried over anhydrous Na₂SO₄, filtered and concentrated to give of 1-isobutyl-6-oxo-1,6-dihydropyridine-3-carboxylic acid (280 mg, yield: 44%) as a yellow solid.

### Step 4. Synthesis of N-(5-bromothiazol-2-yl)-1-isobutyl-6-oxo-1,6-dihydropyridine-3-carboxamide

A mixture of 1-isobutyl-6-oxo-1,6-dihydropyridine-3-carboxylic acid (280 mg, 1.43 mmol), 5-bromothiazol-2-amine (385 mg, 2.15 mmol), T₃P (2.74 g, 4.30 mmol, 50% in EtOAc) and Et₃N (474 mg, 4.68 mmol) in pyridine (2 mL) was stirred at 50° C. for 1 hour. The reaction mixture was concentrated and the residue was purified by flash silica gel chromatography (ISCO®; 12 g SepaFlash® Silica Flash Column, Eluent of 0˜50% EtOAc/PE @45 mL/min) to give N-(5-bromothiazol-2-yl)-1-isobutyl-6-oxo-1,6-dihydropyridine-3-carboxamide (280 mg, yield: 55%) as a yellow solid.

¹H NMR (400 MHz, CD₃OD) δ 0.99 (6H, d, J=6.8 Hz), 2.15-2.30 (1H, m), 3.91 (2H, d, J=7.2 Hz), 6.61 (1H, d, J=9.6 Hz), 7.47 (1H, s), 8.09 (1H, dd, J=9.2, 2.4 Hz), 8.52 (1H, d, J=2.8 Hz).

### Intermediate 6

### 6-chloro-3-iodopyrazolo[1,5-a]pyrazin-4(5H)-one

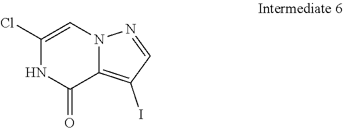 [SMILES: O=c1[nH]c(Cl)cn2ncc(I)c12]

### Step 1. Synthesis of ethyl 1-(cyanomethyl)-1H-pyrazole-5-carboxylate

To a suspension of ethyl 1H-pyrazole-5-carboxylate (40.0 g, 285 mmol) and Cs₂CO₃ (112 g, 343 mmol,) in DMF (500 mL) was added 2-chloroacetonitrile (23.7 g, 314 mmol) at 20-25° C. Then the reaction mixture was stirred at 20-25° C. for 16 hours. The reaction mixture turned into yellow suspension from white. The mixture was filtered through a pad of celite and the solid was washed EtOAc (100 mL×5). The filtrate was concentrated and the residue was poured into water (1000 mL), then extracted with EtOAc (1000 mL×3). The combined organic layer was washed with water (500 mL×2), brine (500 mL), dried over anhydrous Na₂SO₄ and concentrated. The residue was purified by Combi Flash (SiO₂, 10% to 20% EtOAc in PE) to give ethyl 1-(cyanomethyl)-1H-pyrazole-5-carboxylate (32.6 g, yield: 64%) as colorless oil.

### Step 2. Synthesis of ethyl 1-(2-amino-2-oxoethyl)-1H-pyrazole-5-carboxylate

To a solution of ethyl 1-(cyanomethyl)-1H-pyrazole-5-carboxylate (32.0 g, 179 mmol) in TFA (160 mL) was added conc. H₂SO₄ (52 mL) at 20-25° C. Then the reaction mixture was stirred at 20-25° C. for 16 hours. The reaction mixture turned into yellow solution from colorless. The mixture was concentrated and the residue was poured into ice-water (1000 mL), then basified with NaHCO₃ powder to pH=8 and extracted with EtOAc (1000 mL×5). The combined organic layer was washed with brine (1000 mL), dried over anhydrous Na₂SO₄ and concentrated to give ethyl 1-(2-amino-2-oxoethyl)-1H-pyrazole-5-carboxylate (27.3 g, yield: 78%) as a white solid.

### Step 3. Synthesis of pyrazolo[1,5-a]pyrazine-4,6(5H, 7H)-dione

A suspension of ethyl 1-(2-amino-2-oxoethyl)-1H-pyrazole-5-carboxylate (27.0 g, 137 mmol) and t-BuOK (61.5 g, 548 mmol) in toluene (600 mL) was stirred at 110° C. for 2 hours. The reaction mixture turned into yellow suspension from white. The mixture was poured into ice-cold 2N aqueous HCl (250 mL) and stirred for 10 minutes. The precipitate was filtered and washed with water (20 mL×3), dried under vacuum to give pyrazolo[1,5-a]pyrazine-4,6(5H,7H)-dione (15.0 g) as a yellow solid. The filtrate was extracted with EtOAc (500 mL×3). The combined organic layer was washed with brine (100 mL), dried over anhydrous Na₂SO₄ and concentrated to give pyrazolo[1,5-a]pyrazine-4,6(5H,7H)-dione (1.10 g) as a yellow solid. These 2 batches were combined to give pyrazolo[1,5-a]pyrazine-4,6(5H,7H)-dione (16.1 g, yield: 78%) as a yellow solid.

¹H NMR (400 MHz, DMSO-d₆) δ 5.16 (2H, s), 6.97 (1H, d, J=2.0 Hz), 7.75 (1H, d, J=2.0 Hz), 11.81 (1H, brs).

### Step 4. Synthesis of 4,6-dichloropyrazolo[1,5-a]pyrazine

A suspension of pyrazolo[1,5-a]pyrazine-4,6(5H,7H)-dione (16.1 g, 107 mmol) and POCl₃ (245 g, 1.60 mol, 149 mL) was stirred at 95° C. for 48 hours. The reaction mixture turned into black solution from yellow suspension. The mixture was concentrated and the residue was poured into cold water (1000 mL), then basified with NaHCO₃ powder to pH=8 and extracted with EtOAc (1000 mL×3). The combined organic layer was washed with brine (500 mL), dried over anhydrous Na₂SO₄ and concentrated. The residue was purified by Combi Flash (SiO₂, 2% to 10% EtOAc in PE) to give 4,6-dichloropyrazolo[1,5-a]pyrazine (13.8 g, yield: 69%) as a yellow solid.

¹H NMR (400 MHz, CDCl₃) δ 6.86 (1H, d, J=1.6 Hz), 7.99 (1H, d, J=2.4 Hz), 8.36 (1H, s).

### Step 5. Synthesis of 4,6-dichloro-3-iodopyrazolo[1,5-a]pyrazine

A mixture of 4,6-dichloropyrazolo[1,5-a]pyrazine (2.00 g, 10.6 mmol), NIS (4.79 g, 21.3 mmol) in DMF (50 mL) was stirred at 100° C. for 4 hours. The reaction mixture was diluted with saturation aqueous NaHCO₃ (50 mL) and extracted with EtOAc (50 mL×2). The combined organic layers were washed with brine (50 mL×2), dried over anhydrous Na₂SO₄, filtered and concentrated. The residue was purified by Combi Flash (0% to 5% EtOAc in PE) to give 4,6-dichloro-3-iodopyrazolo[1,5-a]pyrazine (3.00 g, yield: 90%) as a white solid.

### Step 6. Synthesis of 6-chloro-3-iodopyrazolo[1,5-a]pyrazin-4(5H)-one

To a solution of 4,6-dichloro-3-iodopyrazolo[1,5-a]pyrazine (3.00 g, 9.56 mmol) in THF (60 mL) was added KOH (6.43 g, 114 mmol) and water (3.6 mL) and the reaction mixture was stirred at 60° C. for 12 hours. The pH of the reaction mixture was adjusted to 7 with 1N aqueous HCl and extracted with EtOAc (50 ml×2). The combined organic layers were dried over anhydrous Na₂SO₄, filtered and concentrated. The residue was purified by Combi Flash (0% to 15% EtOAc in PE) to give 6-chloro-3-iodopyrazolo[1,5-a]pyrazin-4(5H)-one (2.10 g, yield: 74%) as a yellow solid.

### Intermediate 7

### tert-butyl 6,7-dihydrooxazolo[4,5-c]pyridine-5(4H)-carboxylate

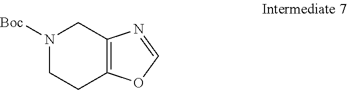 [SMILES: CC(C)(C)OC(=O)N1CCc2ocnc2C1]

### Step 1. Synthesis of ethyl 5-(2-((tert-butoxycarbonyl)amino)ethyl)oxazole-4-carboxylate

To a solution of 3-((tert-butoxycarbonyl)amino)propanoic acid (15.0 g, 79.2 mmol) in THF (200 mL) was added CDI (15.4 g, 95.1 mmol), the mixture was stirred at 55° C. for 2 hours. The reaction mixture was cooled to 0° C., ethyl 2-isocyanoacetate (10.8 g, 95.1 mmol) and DBU (14.5 g, 95.1 mmol) were added and stirred at 25° C. for 12 hours. The reaction mixture was concentrated and the residue was dissolved into EtOAc (500 mL), washed with 10% aqueous citric acid (500 mL), brine (500 mL) dried over Na₂SO₄ and concentrated to give ethyl 5-(2-((tert-butoxycarbonyl)amino)ethyl)oxazole-4-carboxylate (21.0 g, crude) as brown gum, which was used for the next step without purification.

### Step 2. Synthesis of tert-butyl (2-(4-(hydroxymethyl)oxazol-5-yl)ethyl)carbamate

To a solution of ethyl 5-(2-((tert-butoxycarbonyl)amino)ethyl)oxazole-4-carboxylate (7.30 g, crude) in THF (80 mL) was added LiBH₄ (1.33 g, 61.1 mmol) portion-wise at 0° C., the mixture was stirred at 25° C. for 12 hours. The reaction mixture was quenched with saturated aqueous NH₄Cl (20 mL) and concentrated. The residue was purified by flash silica gel chromatography (ISCO®; 80 g SepaFlash® Silica Flash Column, Eluent of 80˜100% Ethyl acetate/Petroleum ether gradient @60 mL/min) to give tert-butyl (2-(4-(hydroxymethyl)oxazol-5-yl)ethyl)carbamate (1.90 g, yield: 28% for two steps) as light yellow gum.

¹H NMR (400 MHz, DMSO-d₆) δ 1.35 (9H, s), 2.79 (2H, t, J=6.8 Hz), 3.05-3.15 (2H, m), 4.29 (2H, d, J=5.6 Hz), 4.96 (1H, t, J=5.6 Hz), 6.88 (1H, t, J=5.2 Hz), 8.14 (1H, s).

### Step 3. Synthesis of tert-butyl 6,7-dihydrooxazolo[4,5-c]pyridine-5(4H)-carboxylate

To a solution of tert-butyl (2-(4-(hydroxymethyl)oxazol-5-yl)ethyl)carbamate (3.20 g, 13.2 mmol) and Et₃N (3.34 g, 33.0 mmol) in DCM (20 mL) was added a solution of MsCl (2.22 g, 19.4 mmol) in DCM (10 mL) dropwise at 0° C., the mixture was stirred at 25° C. for 3.5 hours. The reaction mixture was quenched with saturated aqueous NaHCO₃ (30 mL) and diluted with H₂O (40 mL), then extracted with DCM (50 mL×3). The combined organic layer was washed with brine (60 mL), dried over anhydrous Na₂SO₄, filtered and concentrated. The residue was dissolved in DMF (10 mL) and NaH (537 mg, 13.4 mmol, 60% dispersion in mineral oil) was added at 0° C., the mixture was stirred at 25° C. for 12 hours. The reaction mixture was quenched with MeOH (5 mL) and diluted with H₂O (15 mL), extracted with EtOAc (30 mL×3). The combined organic layer was washed with brine (30 mL), dried over anhydrous Na₂SO₄, filtered and concentrate. The residue was purified by flash silica gel chromatography (ISCO®; 12 g SepaFlash® Silica Flash Column, Eluent of ˜17% Ethyl acetate/Petroleum ether gradient @25 mL/min) to give tert-butyl 6,7-dihydrooxazolo[4,5-c]pyridine-5(4H)-carboxylate (130 mg, yield: 9% for two steps) as colorless oil.

¹H NMR (400 MHz, DMSO-d₆) δ 1.42 (9H, s), 2.71 (2H, t, J=6.0 Hz), 3.67 (2H, t, J=5.6 Hz), 4.30 (2H, s), 8.27 (1H, s).

### Intermediate 8

### 6-chloro-3-iodopyrazolo[1,5-a]pyrazine

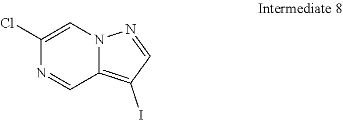 [SMILES: Clc1cn2ncc(I)c2cn1]

### Step 1. Synthesis of 6-chloropyrazolo[1,5-a]pyrazine

A solution of 4,6-dichloropyrazolo[1,5-a]pyrazine (5.00 g, 26.6 mmol), Pd(PPh₃)₄ (3.07 g, 2.66 mmol) and PPh₃ (1.40 g, 5.32 mmol) in toluene (200 mL) was degassed and purged with N₂ for 3 times. Then Bu₃SnH (17.3 g, 59.5 mmol) was added to the reaction mixture under N₂ atmosphere.

The resulting reaction mixture was stirred at 25° C. for 32 hours under N₂ atmosphere. The reaction mixture was concentrated and the residue was diluted with EtOAc (300 mL), then washed with water (100 mL×2), brine (100 mL), dried over anhydrous Na₂SO₄, filtered and concentrated. The residue was purified by silica gel column (0% to 10% EtOAc in PE) to give 6-chloropyrazolo[1,5-a]pyrazine (3.23 g, yield: 79%) as a yellow solid.

### Step 2. Synthesis of 6-chloro-3-iodopyrazolo[1,5-a]pyrazine

A solution of 6-chloropyrazolo[1,5-a]pyrazine (3.20 g, 20.8 mmol) and NIS (9.38 g, 41.7 mmol) in DMF (80 mL) was stirred at 100° C. for 3 hours under N₂ atmosphere. The reaction mixture was concentrated and the residue was diluted with EtOAc (200 mL), then washed with saturated aqueous NaHSO₃ (100 mL×2), saturated aqueous NaHCO₃ (100 mL×2), brine (100 mL), dried over anhydrous Na₂SO₄, filtered and concentrated to give 6-chloro-3-iodopyrazolo[1,5-a]pyrazine (5.68 g, yield: 98%) as a yellow solid.

### Intermediate 9

### 2-ethoxy-4-(4,4,5,5-tetramethyl-1,3,2-dioxaborolan-2-yl)pyridine

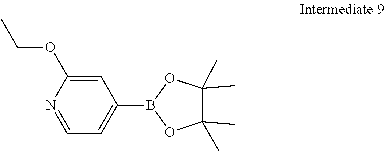 [SMILES: CCOc1cc(B2OC(C)(C)C(C)(C)O2)ccn1]

A mixture of 4-bromo-2-ethoxypyridine (100 mg, 0.494 mmol), Bis-Pin (138 mg, 0.544 mmol), KOAc (146 mg, 1.48 mmol), Pd(dppf)Cl₂ (36 mg, 0.049 mmol) in 1,4-dioxane (4 mL) was degassed and purged with N₂ for 3 times, the reaction mixture was stirred at 90° C. for 12 hours under N₂ atmosphere. The reaction mixture was filtered through a pad of celite. The solid was washed with 1, 4-dioxne (3 mL×2), the filtrate was concentrated to give 2-ethoxy-4-(4,4,5,5-tetramethyl-1,3,2-dioxaborolan-2-yl)pyridine (230 mg, crude) as black brown gum, which was used directly without further purification.

### Intermediate 10

### 7-chloro-3-iodoimidazo[1,2-b]pyridazine

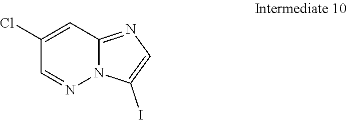 [SMILES: Clc1cnn2c(I)cnc2c1]

### Step 1. Synthesis of 5-chloropyridazin-3-amine

A solution of 3,5-dichloropyridazine (3.00 g, 20.1 mmol) in 28% aqueous N₁₃·H₂O (60 mL) was stirred in a sealed tube at 25° C. for 36 hours. The reaction mixture was concentrated and the residue was purified by silica gel column (0% to 100% EtOAc in PE) to give 5

### Step 2. Synthesis of 7-chloroimidazo[1,2-b]pyridazine

To a solution of 5-chloropyridazin-3-amine (650 mg, 5.02 mmol) and 2-chloroacetaldehyde (4.92 g, 25.1 mmol, 50% in water) in 2-propanol (10 mL) was stirred at 100° C. for 16 hours. The reaction mixture was concentrated and the residue was diluted with H₂O (30 mL) and extracted with EtOAc (50 mL×3). The combined organic layers were washed with brine (100 mL), dried over anhydrous Na₂SO₄, filtered and concentrated. The residue was purified by silica gel column (0% to 60% EtOAc in PE) to give 7-chloroimidazo[1,2-b]pyridazine (70 mg, yield: 9%) as a yellow solid.

¹H NMR (400 MHz, CDCl₃) δ 7.80 (1H, s), 7.95-8.01 (2H, m), 8.30 (1H, d, J=2.0 Hz).

### Step 3. Synthesis of 7-chloro-3-iodoimidazo[1,2-b]pyridazine

To a solution of 7-chloroimidazo[1,2-b]pyridazine (100 mg, 0.650 mmol) in DMF (2 mL) was added NIS (161 mg, 0.720 mmol), then the mixture was stirred at 25° C. for 12 hours. The reaction mixture was poured into water (10 mL) and filtered. The solid was washed with water (5 mL×2) and dried to give 7-chloro-3-iodoimidazo[1,2-b]pyridazine (100 mg, yield: 55%) as a yellow solid.

¹H NMR (400 MHz, CDCl₃) δ 7.87 (1H, s), 7.96 (1H, s), 8.43 (1H, s).

### Intermediate 11

### ethyl 4-(2-bromothiazol-5-yl)benzoate

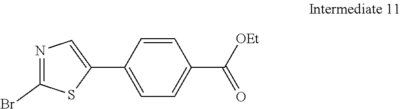 [SMILES: CCOC(=O)c1ccc(-c2cnc(Br)s2)cc1]

### Step 1. Synthesis of ethyl (E)-4-(2-ethoxyvinyl)benzoate

A mixture of ethyl 4-bromobenzoate (2.00 g, 8.73 mmol), 2-[(E)-2-ethoxyvinyl]-4,4,5,5-tetramethyl-1,3,2-dioxaborolane (2.59 g, 13.1 mmol), Pd(dppf)Cl₂ (639 mg, 0.873 mmol) and Na₂CO₃ (2.78 g, 26.2 mmol) in dioxane (20 mL) and H₂O (5 mL) was degassed and purged with N₂ for 3 times. Then the resulting reaction mixture was stirred at 90° C. for 16 hours. The reaction mixture turned into black from red. The reaction mixture was poured into water (50 mL) and EtOAc (50 mL), then filtered through a pad of celite. The aqueous layer was extracted with EtOAc (50 mL×3). The combined organic layer was washed with brine (50 mL), dried over anhydrous Na₂SO₄ and concentrated. The residue was purified by Combi Flash (SiO₂, 5% to 10% EtOAc in PE) to give ethyl (E)-4-(2-ethoxyvinyl)benzoate (1.90 g, yield: 99%) as yellow oil.

¹H NMR (400 MHz, CDCl₃) δ 1.25-1.35 (6H, m), 3.86 (2H, q, J=7.2 Hz), 4.28 (2H, q, J=7.2 Hz), 5.78 (1H, d, J=12.8 Hz), 7.04 (1H, d, J=12.8 Hz), 7.18 (2H, d, J=8.4 Hz), 7.85 (2H, d, J=8.4 Hz).

### Step 2. Synthesis of ethyl 4-(2-aminothiazol-5-yl)benzoate

To a solution of ethyl (E)-4-(2-ethoxyvinyl)benzoate (1.90 g, 8.63 mmol) in dioxane (20 mL) and H₂O (20 mL) was added NBS (1.69 g, 9.49 mmol) at 0° C. Then the reaction mixture was stirred at 25° C. for 30 minutes. Thiourea (722 mg, 9.49 mmol) was added to the reaction mixture and the reaction mixture was stirred at 100° C. for 1 hour. The reaction mixture turned into yellow solution from colorless. The reaction mixture was poured into saturated aqueous NaHCO₃ (100 mL), then extracted with EtOAc (100 mL×3). The combined organic layer was washed with brine (50 mL), dried over anhydrous Na₂SO₄ and concentrated. The crude product was triturated with PE/EtOAc (20 mL, 3/1) to give ethyl 4-(2-aminothiazol-5-yl)benzoate (2.10 g, yield: 98%) as a yellow solid.

¹H NMR (400 MHz, DMSO-d₆) δ 1.31 (3H, t, J=7.2 Hz), 4.30 (2H, q, J=7.2 Hz), 7.38 (2H, brs), 7.53 (2H, d, J=8.4 Hz), 7.60 (1H, s), 7.89 (2H, d, J=8.4 Hz).

### Step 3. Synthesis of ethyl 4-(2-bromothiazol-5-yl)benzoate

To a solution of isoamyl nitrite (142 mg, 1.21 mmol) and CuBr₂ (360 mg, 1.61 mmol) in anhydrous DMF (3 mL) was added ethyl 4-(2-aminothiazol-5-yl)benzoate (200 mg, 0.805 mmol) at 0° C. Then the reaction mixture was stirred at 0° C. for 30 minutes, then heated at 50° C. for 1 hour. The reaction mixture turned into brown solution. The reaction mixture was diluted with water (20 mL), then extracted with EtOAc (20 mL×3). The combined organic layer was washed with water (20 mL×2), brine (20 mL), dried over anhydrous Na₂SO₄ and concentrated. The residue was purified by Combi Flash (SiO₂, 2% to 10% EtOAc in PE) to give ethyl 4-(2-bromothiazol-5-yl)benzoate (240 mg, yield: 95%) as a white solid.

¹H NMR (400 MHz, CDCl₃) δ 1.43 (3H, t, J=7.2 Hz), 4.42 (2H, q, J=7.2 Hz), 7.53-7.62 (2H, m), 7.85 (1H, s), 8.07-8.13 (2H, m).

### Intermediate 12

### tert-butyl 6-methyl-5-oxo-8-(4,4,5,5-tetramethyl-1,3,2-dioxaborolan-2-yl)-3,4,5,6-tetrahydro-2,6-naphthyridine-2(1H)-carboxylate

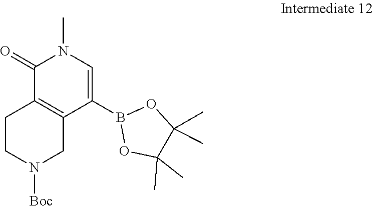 [SMILES: Cn1cc(B2OC(C)(C)C(C)(C)O2)c2c(c1=O)CCN(C(=O)OC(C)(C)C)C2]

### Step 1. Synthesis of (E)-3-(2-(dimethylamino)vinyl)isonicotinonitrile

To a solution of 3-methylisonicotinonitrile (4.66 g, 39.5 mmol) in DMF (50 mL) was added DMF-DMA (9.40 g, 78.9 mmol) at 20° C. The mixture was stirred at 145° C. for 16 hours. The reaction mixture was concentrated to give (E)-3-(2-(dimethylamino)vinyl)isonicotinonitrile (6.80 g, crude) as a brown solid.

### Step 2. Synthesis of 2,6-naphthyridin-1(2H)-one

To a solution of (E)-3-(2-(dimethylamino)vinyl)isonicotinonitrile (6.80 g, crude) in EtOH (70 mL) was added aqueous HBr (46.3 g, 275 mmol, 48% purity) at 20° C. The mixture was stirred at 80° C. for 16 hours. The reaction mixture was concentrated and the residue was diluted with H₂O (100 mL) and extracted with EtOAc (50 mL×3). The combined organic layer was washed with brine (80 mL), dried over anhydrous Na₂SO₄, filtered and concentrated. The residue was purified by flash silica gel chromatography (ISCO®; 40 g SepaFlash® Silica Flash Column, Eluent of 0-100% Ethyl acetate/Petroleum ether gradient @45 mL/min, then 0˜20% MeOH/DCM gradient @45 mL/min) to give 2,6-naphthyridin-1(2H)-one (4.32 g, yield: 75% for two steps) as a light brown solid.

### Step 3. Synthesis of 2-methyl-2,6-naphthyridin-1(2H)-one

To a solution of 2,6-naphthyridin-1(2H)-one (4.32 g, 29.6 mmol) in DMF (50 mL) was added NaH (4.73 g, 118 mmol, 60% dispersion in mineral oil) and Mel (9.44 g, 66.5 mmol) at 0° C. The mixture was stirred at 0° C. for 4 hours and stirred at 20° C. for 18 hours. The reaction mixture was quenched with MeOH (30 mL) at 0° C. and concentrated. The residue was purified by flash silica gel chromatography (ISCO®; 40 g SepaFlash® Silica Flash Column, Eluent of 0˜100% Ethyl acetate/Petroleum ether gradient @50 mL/min) to give 2-methyl-2,6-naphthyridin-1(2H)-one (1.60 g, yield: 34%) as a light yellow solid.

### Step 4. Synthesis of 2-methyl-5,6,7,8-tetrahydro-2,6-naphthyridin-1(2H)-one

A mixture of 2-methyl-2,6-naphthyridin-1(2H)-one (1.60 g, 9.99 mmol) and PtO₂ (1.13 g, 4.99 mmol) in EtOH (20 mL) was degassed and purged with H₂ for 3 times. The mixture was stirred at 20° C. for 18 hours under H₂ atmosphere (50 psi). The reaction mixture was filtered through a pad of celite and the filtrate was concentrated to give 2-methyl-5,6,7,8-tetrahydro-2,6-naphthyridin-1(2H)-one (1.63 g, crude) as a white solid.

### Step 5. Synthesis of tert-butyl 6-methyl-5-oxo-3,4,5,6-tetrahydro-2,6-naphthyridine-2(1H)-carboxylate

To a solution of 2-methyl-5,6,7,8-tetrahydro-2,6-naphthyridin-1(2H)-one (1.63 g, crude) and TEA (3.01 g, 29.8 mmol) in DCM (20 mL) was added Boc₂O (2.38 g, 10.9 mmol). The mixture was stirred at 20° C. for 1 hour. The reaction mixture was diluted with H₂O (50 mL) and extracted with DCM (40 mL×3). The combined organic layers were washed with brine (60 mL), dried over Na₂SO₄, filtered and concentrated. The residue was purified by flash silica gel chromatography (ISCO®; 20 g SepaFlash® Silica Flash Column, Eluent of 0˜80% Ethyl acetate/Petroleum ether gradient @45 mL/min) to give tert-butyl 6-methyl-5-oxo-3,4,5,6-tetrahydro-2,6-naphthyridine-2(1H)-carboxylate (2.49 g, yield: 90% for two steps) as a colorless oil.

### Step 6. Synthesis of tert-butyl 8-bromo-6-methyl-5-oxo-3,4,5,6-tetrahydro-2,6-naphthyridine-2(1H)-carboxylate

To a solution of tert-butyl 6-methyl-5-oxo-3,4,5,6-tetrahydro-2,6-naphthyridine-2(1H)-carboxylate (2.49 g, 9.42 mmol) in MeCN (30 mL) was added NBS (1.84 g, 10.4 mmol). The mixture was stirred at 20° C. for 1 hour. The reaction mixture was concentrated and the residue was diluted with H₂O (50 mL) and extracted with DCM (50 mL×3). The combined organic layers were washed with brine (70 mL), dried over anhydrous Na₂SO₄, filtered and concentrated. The residue was purified by flash silica gel chromatography (ISCO®; 20 g SepaFlash® Silica Flash Column, Eluent of 0˜60% Ethyl acetate/Petroleum ether gradient @45 mL/min) to give tert-butyl 8-bromo-6-methyl-5-oxo-3,4,5,6-tetrahydro-2,6-naphthyridine-2(1H)-carboxylate (3.09 g, yield: 94%) as a light yellow solid

### Step 7. Synthesis of tert-butyl 6-methyl-5-oxo-8-(4,4,5,5-tetramethyl-1,3,2-dioxaborolan-2-yl)-3,4,5,6-tetrahydro-2,6-naphthyridine-2(1H)-carboxylate

To a solution of tert-butyl 8-bromo-6-methyl-5-oxo-3,4,5,6-tetrahydro-2,6-naphthyridine-2(1H)-carboxylate (1.00 g, 2.91 mmol) and Bis-Pin (1.48 g, 5.83 mmol) in 1, 4-dioxane (20 mL) was added KOAc (572 mg, 5.83 mmol), PCy₃ (163 mg, 0.583 mmol) and Pd₂(dba)₃ (267 mg, 0.291 mmol) under N₂ atmosphere, the mixture was stirred at 110° C. for 16 hours under N₂ atmosphere. The reaction mixture was filtered through a pad of celite and the solid was washed with 1, 4-dioxane (20 mL). The filtrate was concentrated and the residue was purified by flash silica gel chromatography (ISCO®; 20 g SepaFlash® Silica Flash Column, Eluent of ˜39% Ethyl acetate/Petroleum ether gradient @30 mL/min) to give tert-butyl 6-methyl-5-oxo-8-(4,4,5,5-tetramethyl-1,3,2-dioxaborolan-2-yl)-3,4,5,6-tetrahydro-2,6-naphthyridine-2(1H)-carboxylate (600 mg, yield: 53%) as a yellow solid.

### Intermediate 13

### N-(5-bromothiazol-2-yl)-1-methyl-6-oxo-1,6-dihydropyridine-3-carboxamide

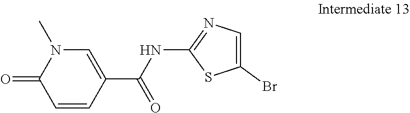 [SMILES: Cn1cc(C(=O)Nc2ncc(Br)s2)ccc1=O]

To a solution of 5-bromothiazol-2-amine (200 mg, 1.31 mmol) and 1-methyl-6-oxo-1,6-dihydropyridine-3-carboxylic acid (281 mg, 1.57 mmol) in pyridine (3 mL) was added T₃P (2.49 g, 3.92 mmol, 50% purity in EtOAc) and Et₃N (396 mg, 3.92 mmol) and stirred at 50° C. for 12 hours. The reaction mixture was concentrated and the residue was basified with saturated aqueous NaHCO₃ to pH=8 and extracted with EtOAc (25 mL×3). The combined organic layer was washed with brine (25 mL) and dried over anhydrous Na₂SO₄ and concentrated. The residue was purified by flash column (SiO₂, 0% to 10% MeOH in DCM) to give N-(5-bromothiazol-2-yl)-1-methyl-6-oxo-1,6-dihydropyridine-3-carboxamide (300 mg, yield: 73%) as a brown solid.

### Intermediate 14

### (1-bromo-9,10-dihydropyrazolo[5,1-f][1,6]naphthyridin-7(8H)-yl)(phenyl)methanone

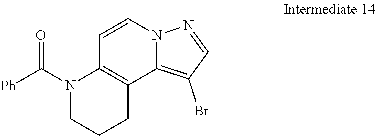 [SMILES: O=C(c1ccccc1)N1CCCc2c1ccn1ncc(Br)c21]

### Step 1. Synthesis of ethyl 5-((tert-butoxycarbonyl)amino)pyrazolo[1,5-a]pyridine-3-carboxylate

A mixture of Int-14a (10.0 g, 37.2 mmol), BocNH₂ (6.53 g, 55.7 mmol), Pd₂(dba)₃ (1.70 g, 1.86 mmol), Xantphos (2.15 g, 3.72 mmol) and Cs₂CO₃ (36.3 g, 111 mmol) in anhydrous 1, 4-dioxane (250 mL) was degassed and purged with N₂ for 3 times. The reaction mixture was stirred at 90° C. for 16 hours under N₂ atmosphere. The reaction mixture was filtered through a pad of celite and the solid was washed with EtOAc (50 mL×4). The filtrate was concentrated and the crude product was triturated with PE/EtOAc (100 mL, 2/1). The mother liquid was concentrated and purified by Combi Flash (SiO₂, 20% to 50% EtOAc in PE). These 2 batches were combined to give ethyl 5-((tert-butoxycarbonyl)amino)pyrazolo[1,5-a]pyridine-3-carboxylate (9.00 g, yield: 97%) as a yellow solid.

### Step 2. Synthesis of ethyl 5-aminopyrazolo[1,5-a]pyridine-3-carboxylate

To a solution of Intermediate 14 (13.0 g, 42.6 mmol) in DCM (100 mL) was added TFA (100 mL). The mixture was stirred at 25° C. for 1 hour. The reaction mixture was concentrated and the residue was diluted with EtOAc (500 mL), washed with saturated aqueous NaHCO₃ (200 mL×2), water (100 mL×2), brine (100 mL), dried over Na₂SO₄, filtered and concentrated to give ethyl 5-aminopyrazolo[1,5-a]pyridine-3-carboxylate (8.50 g, crude) as a yellow solid.

### Step 3. Synthesis of ethyl 5-amino-4-bromopyrazolo[1,5-a]pyridine-3-carboxylate

To a solution of ethyl 5-aminopyrazolo[1,5-a]pyridine-3-carboxylate (6.50 g, 31.7 mmol) in DMF (100 mL) was added NBS (5.64 g, 31.7 mmol). The mixture was stirred at 25° C. for 1 hour. The reaction mixture was diluted with EtOAc (600 mL), washed with saturated aqueous Na₂SO₃ (300 mL×2), saturated aqueous NaHCO₃ (300 mL×2), water (200 mL×2), brine (200 mL), dried over Na₂SO₄ and concentrated. The residue was purified by flash silica gel chromatography (ISCO®; 80 g SepaFlash® Silica Flash Column, Eluent of 0˜45% Ethyl acetate/Petroleum ether gradient @80 mL/min) to give ethyl 5-amino-4-bromopyrazolo[1,5-a]pyridine-3-carboxylate (7.00 g, yield: 58% for two steps) as a yellow solid.

### Step 4. Synthesis of 4-bromopyrazolo[1,5-a]pyridin-5-amine

A solution of ethyl 5-amino-4-bromopyrazolo[1,5-a]pyridine-3-carboxylate (6.00 g, 21.1 mmol) in 48% aqueous HBr (89.4 g, 530 mmol) was stirred at 100° C. for 1 hour. The reaction mixture was poured into ice water and basified with 2N aqueous NaOH to pH=10, extracted with EtOAc (500 mL×3). The combined organic layer was washed with brine (500 mL), dried over anhydrous Na₂SO₄ and concentrated. The residue was purified by flash silica gel chromatography (ISCO®; 40 g SepaFlash® Silica Flash Column, Eluent of 0˜35% Ethyl acetate/Petroleum ether gradient @50 mL/min) to give 4-bromopyrazolo[1,5-a]pyridin-5-amine (3.10 g, yield: 69%) as a gray solid.

### Step 5. Synthesis of N-(4-bromopyrazolo[1,5-a]pyridin-5-yl)benzamide

To a solution of 4-bromopyrazolo[1,5-a]pyridin-5-amine (2.60 g, 12.3 mmol) and DIPEA (3.17 g, 24.5 mmol) in anhydrous DCM (50 mL) was added benzoyl chloride (2.07 g, 14.7 mmol) at 0° C. The reaction mixture was stirred at 0° C. for 1 hour. The reaction mixture was concentrated and the residue was purified by flash silica gel chromatography (ISCO®; 20 g SepaFlash® Silica Flash Column, Eluent of 0-22% Ethyl acetate/Petroleum ether gradient @40 mL/min) to give N-(4-bromopyrazolo[1,5-a]pyridin-5-yl)benzamide (3.30 g, yield: 85%) as a yellow solid.

### Step 6. Synthesis of N-(4-vinylpyrazolo[1,5-a]pyridin-5-yl)benzamide

To a solution of N-(4-bromopyrazolo[1,5-a]pyridin-5-yl)benzamide (1.60 g, 5.06 mmol) and trifluoro(vinyl)-2-borane, potassium salt (1.02 g, 7.59 mmol) in 1,4-dioxane (20 mL) and H₂O (4 mL) was added Xphos-Pd-G₃ (428 mg, 0.506 mmol) and Na₂CO₃ (1.07 g, 10.1 mmol) under N₂ atmosphere, the mixture was stirred at 90° C. for 16 hours under N₂ atmosphere. The reaction mixture was concentrated and the residue was diluted with H₂O (50 mL), extracted with DCM (50 mL×3). The combined organic layer was concentrated and the residue was purified by flash silica gel chromatography (ISCO®; 20 g SepaFlash® Silica Flash Column, Eluent of ˜42% Ethyl acetate/Petroleum ether gradient @40 mL/min) to give N-(4-vinylpyrazolo[1,5-a]pyridin-5-yl)benzamide (700 mg, yield: 53%) as a yellow solid.

¹H NMR (400 MHz, DMSO-d₆) δ 5.59-5.66 (1H, m), 6.03 (1H, dd, J=18.0, 0.8 Hz), 6.89-7.02 (3H, m), 7.51-7.59 (2H, m), 7.59-7.67 (1H, m), 7.97-8.04 (2H, m), 8.06 (1H, d, J=2.0 Hz), 8.64 (1H, d, J=7.2 Hz), 10.26 (1H, s).

### Step 7. Synthesis of N-allyl-N-(4-vinylpyrazolo[1,5-a]pyridin-5-yl)benzamide

To a solution of NaH (301 mg, 7.52 mmol, 60% dispersion in mineral oil) in anhydrous DMF (5 mL) was added N-(4-vinylpyrazolo[1,5-a]pyridin-5-yl)benzamide (660 mg, 2.51 mmol) in DMF (5 mL) dropwise at 0° C., the mixture was stirred at 0° C. for 1 hour. 3-bromoprop-1-ene (607 mg, 5.01 mmol) in DMF (5 mL) was added to the reaction mixture dropwise at 0° C., the mixture was stirred at 20° C. for 3 hours. The reaction mixture was quenched with H₂O (60 mL) at 0° C., extracted with EtOAc (60 mL×2). The combined organic layer was dried over anhydrous Na₂SO₄, filtered and concentrated. The residue was purified by flash silica gel chromatography (ISCO®; 20 g SepaFlash® Silica Flash Column, Eluent of ˜18% Ethyl acetate/Petroleum ether gradient @40 mL/min) to give N-allyl-N-(4-vinylpyrazolo[1,5-a]pyridin-5-yl)benzamide (730 mg, yield: 93%) as a yellow gum.

### Step 8. Synthesis of phenyl(pyrazolo[5,1-f][1,6]naphthyridin-7(8H)-yl)methanone

A solution of N-allyl-N-(4-vinylpyrazolo[1,5-a]pyridin-5-yl)benzamide (182 mg, 0.600 mmol) and Grubbs II catalyst (56 mg, 0.090 mmol) in DCM (16 mL) was bubbled with N₂ for 6 minutes, then stirred at 60° C. for 1 hour under microwave irradiation. The reaction mixture was concentrated and the residue was purified by flash silica gel chromatography (ISCO®; 20 g SepaFlash® Silica Flash Column, Eluent of ˜28% Ethyl acetate/Petroleum ether gradient @40 mL/min) to give phenyl(pyrazolo[5,1-f][1,6]naphthyridin-7(8H)-yl)methanone (510 mg, yield: 76%) as a yellow solid.

### Step 9. Synthesis of (9,10-dihydropyrazolo[5,1-f][1,6]naphthyridin-7(8H)-yl)(phenyl)methanone

A mixture of phenyl(pyrazolo[5,1-f][1,6]naphthyridin-7(8H)-yl)methanone (510 mg, 1.85 mmol) and 10% Pd/C (300 mg) in MeOH (30 mL) was degassed and purged with H₂ for three times, the mixture was stirred at 20° C. for 3 hours under H₂ (15 psi) atmosphere. The reaction mixture was filtered through a pad of celite and the solid was washed with MeOH (10 mL×3). The filtrate was concentrated to give (9,10-dihydropyrazolo[5,1-f][1,6]naphthyridin-7(8H)-yl)(phenyl)methanone (440 mg, yield: 83%) as white solid.

### Step 10. Synthesis of (1-bromo-9,10-dihydropyrazolo[5,1-f][1,6]naphthyridin-7(8H)-yl)(phenyl)methanone

To a solution of (9,10-dihydropyrazolo[5,1-f][1,6]naphthyridin-7(8H)-yl)(phenyl)methanone (410 mg, 1.48 mmol) in DMF (6 mL) was added NBS (289 mg, 1.63 mmol) at 0° C., the mixture was stirred at 20° C. for 3 hours. The reaction mixture was concentrated and the residue was purified by flash silica gel chromatography (ISCO®; 12 g SepaFlash® Silica Flash Column, Eluent of 19-69% Ethyl acetate/Petroleum ether gradient @30 mL/min) to give (1-bromo-9,10-dihydropyrazolo[5,1-f][1,6]naphthyridin-7(8H)-yl)(phenyl)methanone (650 mg, yield: 99%) as a white solid.

### Intermediate 15

### tert-butyl 4-((5-bromothiazol-2-yl)ethynyl)piperidine-1-carboxylate

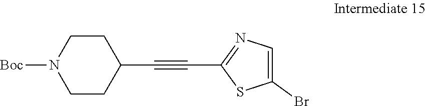 [SMILES: CC(C)(C)OC(=O)N1CCC(C#Cc2ncc(Br)s2)CC1]

A mixture of 5-bromo-2-iodothiazole (2.00 g, 6.90 mmol), tert-butyl 4-ethynylpiperidine-1-carboxylate (1.73 g, 8.28 mmol), Pd(PPh₃)₂Cl₂ (484 mg, 0.689 mmol), CuI (263 mg, 1.38 mmol) and Et₃N (4.8 mL) in THF (20 mL) was degassed and purged with N₂ for 3 times, then the mixture was stirred at 20° C. for 3 hours under N₂ atmosphere. The reaction mixture was concentrated and the residue was purified by silica gel column (PE/EtOAc=5/1) to afford tert-butyl 4-((5-bromothiazol-2-yl)ethynyl)piperidine-1-carboxylate (1.56 g, yield: 60%) as yellow gum.

¹H NMR (400 MHz, DMSO-d₆) δ 1.38 (9H, s), 1.68-1.77 (2H, m), 1.81-1.86 (2H, m), 2.68-2.78 (1H, m), 3.01-3.11 (4H, m), 7.95 (1H, s).

### Compounds of Formula (II)

### Example 1

### N-(5-(4-cyanophenyl)thiazol-2-yl)-6-oxopiperidine-3-carboxamide

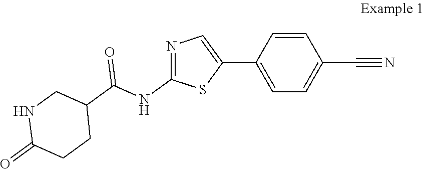 [SMILES: N#Cc1ccc(-c2cnc(NC(=O)C3CCC(=O)NC3)s2)cc1]

To a solution of Int-1 (60 mg, 0.30 mmol), 6-oxopiperidine-3-carboxylic acid (43 mg, 0.30 mmol) in pyridine (1 mL) was added T₃P (379 mg, 0.596 mmol, 50% in EtOAc). The mixture was stirred at 25° C. for 2 hours. The reaction mixture was concentrated and the residue was diluted with H₂O (20 mL) and extracted with EtOAc (20 mL×3). The combined organic layers were washed with brine (40 mL), dried over anhydrous Na₂SO₄, filtered and concentrated. The residue was purified by prep-HPLC (0.225% FA as an additive), then lyophilized to afford the title compound (15.0 mg, yield: 8%) as a white solid.

¹H NMR (400 MHz, DMSO-d₆) δ 1.85-1.97 (1H, m), 1.98-2.09 (1H, m), 2.15-2.29 (2H, m), 2.90-3.01 (1H, m), 3.22-3.31 (2H, m), 7.54 (1H, brs), 7.81 (2H, d, J=8.4 Hz), 7.86 (2H, d, J=8.4 Hz), 8.12 (1H, s), 12.50 (1H, brs).

The following compounds were synthesized analogously to Example 1

| Example | | | |
|---|---|---|---|
| No. | Structure | Name | ¹H NMR (400 MHz) |
| 2 | 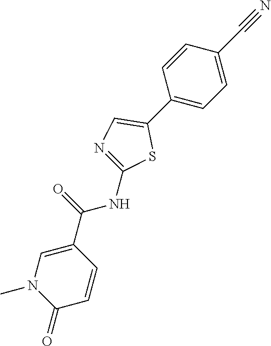 [SMILES: Cn1cc(C(=O)Nc2ncc(-c3ccc(C#N)cc3)s2)ccc1=O] | N-(5-(4- cyanophenyl)thiazol-2- yl)-1-methyl-6-oxo- 1,6-dihydropyridine-3- carboxamide | DMSO-d₆; δ 3.52 (3H, s), 6.48 (1H, d, J = 9.6 Hz), 7.82-7.90 (4H, m), 8.05 (1H, dd, J = 9.6, 2.8 Hz), 8.19 (1H, s), 8.76 (1H, s), 12.57 (1H, brs). |
| 6 | 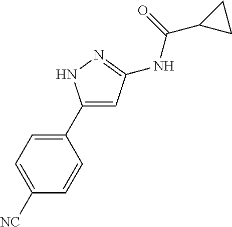 [SMILES: N#Cc1ccc(-c2cc(NC(=O)C3CC3)n[nH]2)cc1] | N-(5-(4-cyanophenyl)- 1H-pyrazol-3- yl)cyclopropane- carboxamide | DMSO-d₆) δ 0.68-0.91 (4H, m), 1.80-1.90 (1H, m), 6.93 (1H, s), 7.90-7.95 (4H, m), 10.82 (1H, brs), 13.62 (1H, brs). |
| 12 | 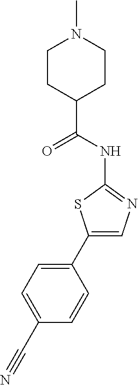 [SMILES: CN1CCC(C(=O)Nc2ncc(-c3ccc(C#N)cc3)s2)CC1] | N-(5-(4- cyanophenyl)thiazol-2- yl)-1-methylpiperidine- 4-carboxamide | DMSO-d₆; δ 1.55-1.69 (2H, m), 1.72-1.89 (4H, m), 2.14 (3H, s), 2.39-2.45 (1H, m), 2.77-2.82 (2H, m), 7.78 (2H, d, J = 8.4 Hz), 7.84 (2H, d, J = 8.4 Hz), 8.09 (1H, s), 12.31 (br s, 1H, brs). |
| 13 | 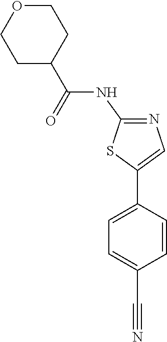 [SMILES: N#Cc1ccc(-c2cnc(NC(=O)C3CCOCC3)s2)cc1] | N-(5-(4- cyanophenyl)thiazol-2- yl)tetrahydro-2H- pyran-4-carboxamide | DMSO-d₆; δ 1.58-1.81 (4H, m), 2.73-2.84 (1H, m), 3.38- 3.49 (2H, m), 3.84-3.96 (2H, m), 7.81 (2H, d, J = 8.4 Hz), 7.86 (2H, d, J = 8.4 Hz), 8.12 (1H, s), 12.37 (1H, brs). |
| 14 | 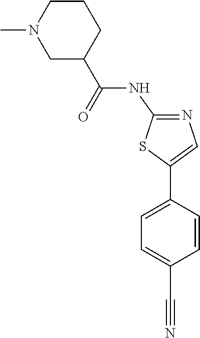 [SMILES: CN1CCCC(C(=O)Nc2ncc(-c3ccc(C#N)cc3)s2)C1] | N-(5-(4- cyanophenyl)thiazol-2- yl)-1-methylpiperidine- 3-carboxamide | DMSO-d₆; δ 1.28-1.58 (2H, m), 1.59-2.14 (4H, m), 2.18 (3H, s), 2.65-2.70 (1H, m), 2.73-2.86 (2H, m), 7.81 (2H, d, J = 8.0 Hz), 7.86 (2H, d, J = 8.4 Hz), 8.11 (1H, s), 12.38 (1H, brs). |
| 55 | 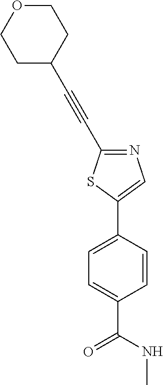 [SMILES: CNC(=O)c1ccc(-c2cnc(C#CC3CCOCC3)s2)cc1] | N-methyl-4-(2- ((tetrahydro-2H-pyran- 4-yl)ethynyl)thiazol-5- yl)benzamide | DMSO-d₆; δ 1.59-1.71 (2H, m), 1.83-1.93 (2H, m), 2.79 (3H, d, J = 4.4 Hz), 2.99-3.10 (1H, m), 3.40-3.51 (2H, m), 3.73-3.85 (2H, m), 7.78 (2H, d, J = 8.4 Hz), 7.90 (2H, d, J = 8.4 Hz), 8.38 (1H, s), 8.52 (1H, t, J = 4.8 Hz). |
| 57 | 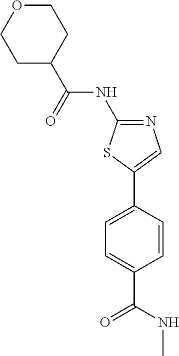 [SMILES: CNC(=O)c1ccc(-c2cnc(NC(=O)C3CCOCC3)s2)cc1] | N-(5-(4- (methylcarbamoyl) phenyl)thiazol-2- yl)tetrahydro-2H- pyran-4-carboxamide | DMSO-d₆) δ 1.58-1.82 (4H, m), 2.73-2.84 (4H, m), 3.36- 3.43 (2H, m), 3.87-3.95 (2H, m), 7.70 (2H, d, J = 8.4 Hz), 7.87 (2H, d, J = 8.4 Hz), 8.00 (1H, s), 8.46 (1H, t, J = 4.4 Hz), 12.24 (1H, brs). |

### Example 3

### N-(4-(3-cyanophenyl)pyridin-2-yl)-1-isobutylpiperidine-4-carboxamide

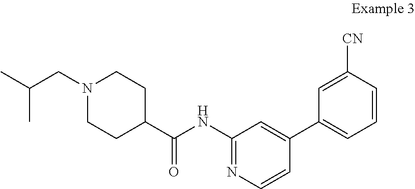 [SMILES: CC(C)CN1CCC(C(=O)Nc2cc(-c3cccc(C#N)c3)ccn2)CC1]

### Step 1. Synthesis of tert-butyl 4-((4-(3-cyanophenyl)pyridin-2-yl)carbamoyl)piperidine-1-carboxylate

A mixture of Int-2 (180 mg, 0.468 mmol), 3-a (100 mg, 0.681 mmol), Pd(dppf)Cl₂ (33 mg, 0.045 mmol) and K₂CO₃ (125 mg, 0.907 mmol) in dioxane (4 mL) and H₂O (1 mL) was degassed and purged with N₂ for 3 times, then stirred at 80° C. for 12 hours under N₂ atmosphere. The reaction mixture was diluted with H₂O (25 mL) and extracted with EtOAc (25 mL×3). The combined organic layers were washed with brine (25 mL), dried over anhydrous Na₂SO₄, filtered and concentrated. The residue was purified by column chromatography (SiO₂, Petroleum ether/Ethyl acetate=1/1 to 1/2) to give tert-butyl 4-((4-(3-cyanophenyl)pyridin-2-yl)carbamoyl)piperidine-1-carboxylate (150 mg, yield: 81%) as a colorless gum.

### Step 2. Synthesis of N-(4-(3-cyanophenyl)pyridin-2-yl)piperidine-4-carboxamide

A solution of tert-butyl 4-((4-(3-cyanophenyl)pyridin-2-yl)carbamoyl)piperidine-1-carboxylate (150 mg, 0.369 mmol) in 4N HCl/EtOAc (5 mL) was stirred at 25° C. for 1 hour. The reaction mixture was concentrated to give N-(4-(3-cyanophenyl)pyridin-2-yl)piperidine-4-carboxamide (120 mg, yield: 95%, HCl salt) as a white solid.

### Step 3. Synthesis of N-(4-(3-cyanophenyl)pyridin-2-yl)-1-isobutylpiperidine-4-carboxamide

To a solution of N-(4-(3-cyanophenyl)pyridin-2-yl)piperidine-4-carboxamide (120 mg, 0.392 mmol, HCl salt) in MeOH (3 mL) was added isobutyraldehyde (56 mg, 0.78 mmol) at 25° C. The mixture was stirred at 25° C. for 0.5 hour. NaBH₃CN (74 mg, 1.2 mmol) was added to the reaction mixture at 25° C. Then the mixture was stirred at 25° C. for another 0.5 hour. The reaction mixture was quenched with H₂O (25 mL) and extracted with EtOAc (25 mL×3). The combined organic layers were washed with brine (25 mL), dried over anhydrous Na₂SO₄, filtered and concentrated. The residue was purified by prep-HPLC (0.05% NH₃·H₂O as an additive) and lyophilized to give the title compound (72.22 mg, yield: 50%) as a white solid.

¹H NMR (400 MHz, CD₃OD) δ 0.93 (6H, d, J=6.4 Hz), 1.80-1.94 (5H, m), 1.96-2.08 (2H, m), 2.10-2.15 (2H, m), 2.44-2.58 (1H, m), 2.95-3.05 (2H, m), 7.42 (1H, dd, J=5.2, 1.6 Hz), 7.66-7.73 (1H, m), 7.82 (1H, d, J=8.0 Hz), 8.01-8.06 (1H, m), 8.11 (1H, d, J=1.6 Hz), 8.42 (1H, s), 8.38 (1H, d, J=5.2 Hz).

The following compound was synthesized analogously to Example 3

| Example | | | |
|---|---|---|---|
| No. | Structure | Name | ¹H NMR (400 MHz) |
| 4 | 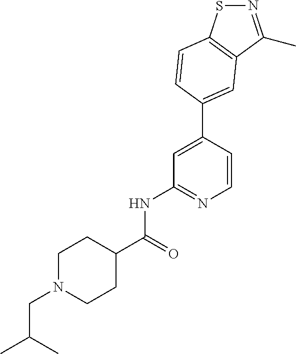 [SMILES: Cc1nsc2ccc(-c3ccnc(NC(=O)C4CCN(CC(C)C)CC4)c3)cc12] | 1-isobutyl-N-(4-(3- methylbenzo[d] isothiazol- 5-yl)pyridin-2- yl)piperidine-4- carboxamide | CD₃OD; δ 0.96 (6H, d, J = 6.8 Hz), 1.83-1.97 (5H, m), 2.01- 2.11 (2H, m), 2.15-2.20 (2H, m), 2.47-2.61 (1H, m), 2.84 (s, 3H), 3.00-3.05 (2H, m), 7.55 (1H, dd, J = 5.6, 1.6 Hz), 7.97 (1H, dd, J = 8.4, 1.6 Hz), 8.18 (1H, d, J = 8.4 Hz), 8.38-8.47 (2H, m), 8.54 (1H, s). |

### Example 5

### N-(5-(4-cyanophenyl)-1H-pyrazol-3-yl)indoline-2-carboxamide

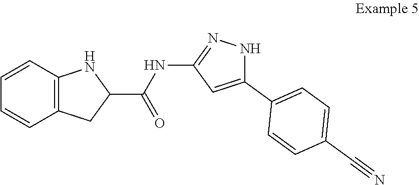 [SMILES: N#Cc1ccc(-c2cc(NC(=O)C3Cc4ccccc4N3)n[nH]2)cc1]

### Step 1. Synthesis of tert-butyl 2-((5-(4-cyanophenyl)-1H-pyrazol-3-yl)carbamoyl)indoline-1-carboxylate

To a solution of Int-4 (150 mg, 0.814 mmol) and 4-a (279 mg, 1.06 mmol) in pyridine (3 mL) was added EDCI (234 mg, 1.22 mmol), the mixture was stirred at 20° C. for 2 hours. The reaction mixture was concentrated and the residue was diluted with DCM (30 mL), washed with water (30 mL), brine (30 mL), dried over anhydrous Na₂SO₄, filtered and concentrated. The residue was purified by flash silica gel chromatography (ISCO®; 20 g SepaFlash® Silica Flash Column, Eluent of 0˜60% Ethyl acetate/Petroleum ether gradient @25 mL/min) to give tert-butyl 2-((5-(4-cyanophenyl)-1H-pyrazol-3-yl)carbamoyl)indoline-1-carboxylate (160 mg, yield: 46%) as a white solid.

¹H NMR (400 MHz, DMSO-d₆) δ 1.27-1.64 (9H, m), 2.92-3.11 (1H, m), 3.41-3.59 (1H, m), 4.87-5.03 (1H, m), 6.85-7.08 (2H, m), 7.12-7.25 (2H, m), 7.66-8.04 (5H, m), 10.84 (1H, brs), 13.17 (1H, brs).

### Step 2. Synthesis of N-(5-(4-cyanophenyl)-1H-pyrazol-3-yl)indoline-2-carboxamide

To a solution of tert-butyl 2-((5-(4-cyanophenyl)-1H-pyrazol-3-yl)carbamoyl)indoline-1-carboxylate (60 mg, 0.14 mmol) in EtOAc (5 mL) was added 4N HCl/EtOAc (5 mL) at 0° C., the mixture was stirred at 0° C. for 1 hour and stirred at 20° C. for 11 hours. The reaction mixture was concentrated to give the title compound (45 mg, yield: 83%, HCl salt) as an off-white solid.

¹H NMR (400 MHz, CD₃OD) δ 3.36-3.52 (1H, m), 3.68-3.90 (1H, m), 4.93-5.04 (1H, m), 7.00 (1H, s), 7.20-7.46 (4H, m), 7.76-7.90 (4H, m).

### Example 7

### 4-(1-methyl-2-oxo-2,3-dihydro-1H-imidazo[4,5-b]pyridin-6-yl)benzoic acid

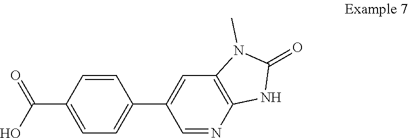 [SMILES: Cn1c(=O)[nH]c2ncc(-c3ccc(C(=O)O)cc3)cc21]

### Step 1. Synthesis of tert-butyl 4-(I-methyl-2-oxo-3-trityl-2,3-dihydro-1H-imidazo[4,5-b]pyridin-6-yl)benzoate

To a solution of 6-bromo-1-methyl-3-trityl-1,3-dihydro-2H-imidazo[4,5-b]pyridin-2-one (300 mg, 0.638 mmol), (4-(tert-butoxycarbonyl)phenyl)boronic acid (213 mg, 0.701 mmol) in dioxane (8 mL) and H₂O (1 mL) was added Na₂CO₃ (203 mg, 1.91 mmol) and Pd(dppf)Cl₂ (47 mg, 0.064 mmol), then the mixture was degassed and purged with N₂ for 3 times and the mixture was stirred at 90° C. for 12 hours under N₂ atmosphere. The reaction mixture was suspended in CH₃OH (20 mL) and filtered. The filtrate was concentrated and the residue was purified by silica gel column (0% to 100% EtOAc in PE) to give tert-butyl 4-(1-methyl-2-oxo-3-trityl-2,3-dihydro-1H-imidazo[4,5-b]pyridin-6-yl)benzoate (250 mg, yield: 69%) as a white solid.

¹H NMR (400 MHz, CDCl₃) δ 1.62 (9H, s), 3.39 (3H, s), 7.17-7.22 (3H, m), 7.23-7.27 (6H, m), 7.28 (1H, s), 7.51-7.56 (7H, m), 7.56-7.57 (1H, m), 8.03 (1H, s), 8.03-8.07 (2H, m).

### Step 2. Synthesis of 4-(I-methyl-2-oxo-2,3-dihydro-1H-imidazo[4,5-b]pyridin-6-yl)benzoic acid

To a solution of tert-butyl 4-(1-methyl-2-oxo-3-trityl-2,3-dihydro-1H-imidazo[4,5-b]pyridin-6-yl)benzoate (250 mg, 0.440 mol) in DCM (5 mL) was added TFA (2 mL). The mixture was stirred at 25° C. for 1 hour. The reaction mixture was concentrated and the crude product was triturated with MeOH (5 mL) and MeCN (2 mL) to afford the title compound (93.67 mg, yield: 77%) as an off-white solid.

¹H NMR (400 MHz, DMSO-d₆) δ 3.37 (3H, s), 7.82-7.86 (2H, m), 7.86-7.88 (1H, m), 8.03 (2H, d, J=8.4 Hz), 8.32 (1H, d, J=2.0 Hz), 11.69 (1H, brs), 13.00 (1H, brs).

The following compounds were synthesized analogously to Example 7

| Example | | | |
|---|---|---|---|
| No. | Structure | Name | ¹H NMR (400 MHz) |
| 8 | 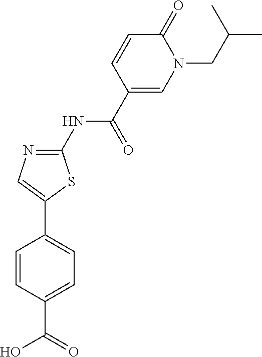 [SMILES: CC(C)Cn1cc(C(=O)Nc2ncc(-c3ccc(C(=O)O)cc3)s2)ccc1=O] | 4-(2-(1-isobutyl-6-oxo- 1,6-dihydropyridine-3- carboxamido)thiazol-5- yl)benzoic acid | DMSO-d₆; δ 0.90 (6H, d, J = 6.8 Hz), 2.10-2.20 (1H, m), 3.81 (2H, d, J = 7.6 Hz), 6.50 (1H, d, J = 9.6 Hz), 7.77 (2H, d, J = 8.4 Hz), 7.97 (2H, d, J = 8.4 Hz), 8.04 (1H, dd, J = 9.6, 2.4 Hz), 8.12 (1H, s), 8.70 (1H, d, J = 2.8 Hz), 12.15 (1H, brs), 12.76 (1H, brs). |
| 19 | 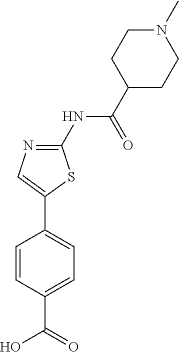 [SMILES: CN1CCC(C(=O)Nc2ncc(-c3ccc(C(=O)O)cc3)s2)CC1] | 4-(2-(1- methylpiperidine-4- carboxamido)thiazol-5- yl)benzoic acid | DMSO-d₆; δ 1.60-1.73 (2H, m), 1.76-1.84 (2H, m), 1.87-1.98 (2H, m), 2.19 (3H, s), 2.46-2.49 (1H, m), 2.80-2.85 (2H, m), 7.71 (2H, d, J = 8.4 Hz), 7.95 (2H, d, J = 8.4 Hz), 8.02 (1H, s), 12.27 (1H, brs). |
| 58 | 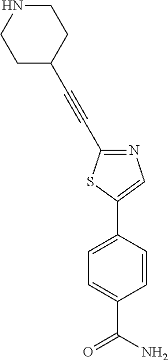 [SMILES: NC(=O)c1ccc(-c2cnc(C#CC3CCNCC3)s2)cc1] | 4-(2-(piperidin-4- ylethynyl)thiazol-5- yl)benzamide | DMSO-d₆; δ 1.44-1.57 (2H, m), 1.75-1.84 (2H, m), 2.52-2.58 (2H, m), 2.80-2.91 (3H, m), 7.44 (1H, brs), 7.75 (2 H, d, J = 8.4 Hz), 7.92 (2H, d, J = 8.8 Hz), 8.03 (1H, brs), 8.35 (1H, s). |

### Example 9

### N-(5-(4-cyano-2-methylphenyl)thiazol-2-yl)-1-methyl-6-oxo-1,6-dihydropyridine-3-carboxamide

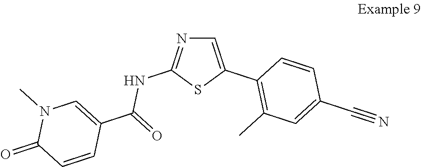 [SMILES: Cc1cc(C#N)ccc1-c1cnc(NC(=O)c2ccc(=O)n(C)c2)s1]

A mixture of Int-13 (100 mg, 0.318 mmol), (4-cyano-2-methylphenyl)boronic acid (102 mg, 0.637 mmol), Pd(dtbpf)Cl₂ (21 mg, 0.032 mmo) and Na₂CO₃ (101 mg, 0.955 mmol) in dioxane (3 mL) and H₂O (0.75 mL) was degassed and purged with N₂ for 3 times, then stirred at 90° C. for 16 hours under N₂ atmosphere. The reaction mixture was filtered and the filtrate was concentrated. The residue was purified by prep-HPLC (Method C; 0.22500 FA as an additive) and lyophilized to give the title compound (9.37 mg, yield: 80%) as an off-white solid.

¹H NMR (400 MHz, DMSO-d₆) 2.53 (3H, s), 3.53 (3H, s), 6.48 (1H, d, J=9.6 Hz), 7.61-7.66 (1H, m), 7.70-7.75 (1H, m), 7.80 (1H, s), 7.84 (1H, s), 8.06 (1H, dd, J=9.2, 2.4 Hz), 8.75 (1H, d, J=2.4 Hz).

The following compounds were synthesized analogously to Example 9

| Example | | | |
|---|---|---|---|
| No. | Structure | Name | ¹H NMR (400 MHz) |
| 10 | 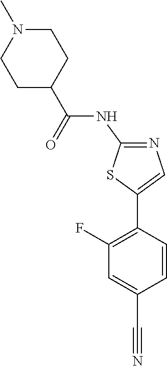 [SMILES: CN1CCC(C(=O)Nc2ncc(-c3ccc(C#N)cc3F)s2)CC1] | N-(5-(4-cyano-2- fluorophenyl)thiazol- 2-yl)-1- methylpiperidine-4- carboxamide | DMSO-d₆; δ 1.60-1.72 (2H, m), 1.80-1.85 (2H, m), 1.86-1.94 (2H, m), 2.20 (3H, s), 2.40-2.45 (1H, m), 2.80-2.85 (2H, m), 7.75 (1H, dd, J = 8.0, 1.6 Hz), 7.97- 8.05 (2H, m), 8.17 (1H, s). |
| 11 | 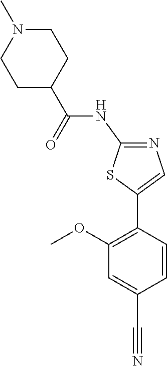 [SMILES: COc1cc(C#N)ccc1-c1cnc(NC(=O)C2CCN(C)CC2)s1] | N-(5-(4-cyano-2- methoxyphenyl) thiazol-2-yl)-1- methylpiperidine-4- carboxamide | DMSO-d₆; δ 1.59-1.72 (2H, m), 1.74-1.80 (2H, m), 1.82-1.92 (2H, m), 2.16 (3H, s), 2.40-2.45 (1H, m), 2.80-2.90 (2H, m), 3.99 (3H, s), 7.47 (1H, dd, J = 8.0, 1.2 Hz), 7.63 (1H, s), 7.93 (1H, d, J = 8.0 Hz), 8.16 (1H, s), 12.17 (1H, brs). |
| 15 | 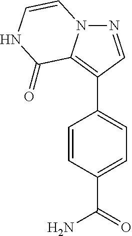 [SMILES: NC(=O)c1ccc(-c2cnn3cc[nH]c(=O)c23)cc1] | 4-(4-oxo-4,5- dihydropyrazolo[1,5- a]pyrazin-3- yl)benzamide | DMSO-d₆; δ 6.92 (1H, d, J = 5.6 Hz), 7.37 (1H, brs), 7.69 (1H, d, J = 5.6 Hz), 7.89 (2H, d, J = 8.4 Hz), 7.96 (2H, d, J = 8.8 Hz), 8.01 (1H, brs), 8.25 (1H, s). |
| 17 | 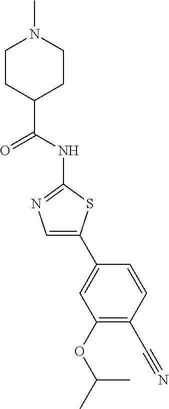 [SMILES: CC(C)Oc1cc(-c2cnc(NC(=O)C3CCN(C)CC3)s2)ccc1C#N] | N-(5-(4-cyano-3- isopropoxyphenyl) thiazol-2-yl)-1- methylpiperidine-4- carboxamide | DMSO-d₆; δ 1.34 (6H, d, J = 6.0 Hz), 1.59-1.73 (2H, m), 1.76- 1.84 (2H, m), 1.86-1.99 (2H, m), 2.18 (3H, s), 2.41-2.49 (1H, m), 2.80-2.85 (2H, m), 4.95-5.00 (1H, m), 7.27 (1H, d, J = 8.0 Hz), 7.42 (1H, s), 7.72 (1H, d, J = 8.0 Hz), 8.16 (1H, s), 12.35 (1H, brs). |
| 46 | 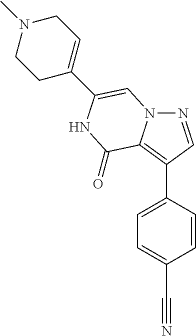 [SMILES: CN1CC=C(c2cn3ncc(-c4ccc(C#N)cc4)c3c(=O)[nH]2)CC1] | 4-(6-(1-methyl- 1,2,3,6- tetrahydropyridin-4- yl)-4-oxo-4,5- dihydropyrazolo[1,5- a]pyrazin-3- yl)benzonitrile | DMSO-d₆; δ 2.28 (3H, s), 2.40- 2.45 (2H, m), 2.53-2.56 (2H, m), 3.00-3.05 (2H, m), 6.50- 6.60 (1H, m), 7.75 (1H, s), 7.89 (2H, d, J = 8.4 Hz), 8.16 (2H, d, J = 8.4 Hz), 8.38 (1H, s), 11.19 (1H, brs). |

### Example 16

### 4-(6-(4-fluorophenyl)-5-methyl-4-oxo-4,5-dihydropyrazolo[1,5-a]pyrazin-3-yl)benzonitrile

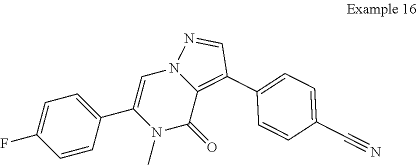 [SMILES: Cn1c(-c2ccc(F)cc2)cn2ncc(-c3ccc(C#N)cc3)c2c1=O]

### Step 1. Synthesis of 4-(6-chloro-4-oxo-4,5-dihydropyrazolo[1,5-a]pyrazin-3-yl)benzonitrile

A mixture of Int-6 (500 mg, 1.69 mmol), 4-cyanophenylboronic acid (299 mg, 2.03 mmol) and Pd(dppf)Cl₂ (124 mg, 0.169 mmol), Na₂CO₃ (359 mg, 3.38 mmol) in 1,4-dioxane (8 mL) and water (1 mL) was degassed and purged with N₂ for 3 times, and then the mixture was stirred at 90° C. for 12 hours under N₂ atmosphere. The reaction mixture was concentrated and the residue was purified by Combi Flash (0% to 100% EtOAc in PE) to give 4-(6-chloro-4-oxo-4,5-dihydropyrazolo[1,5-a]pyrazin-3-yl)benzonitrile (220 mg, yield: 48%) as a yellow solid.

¹H NMR (400 MHz, DMSO-d₆) δ 7.86-7.92 (2H, m), 8.07-8.11 (2H, m), 8.13 (1H, s), 8.36 (1H, s).

### Step 2. Synthesis of 4-(6-chloro-5-methyl-4-oxo-4,5-dihydropyrazolo[1,5-a]pyrazin-3-yl)benzonitrile

To a solution of 4-(6-chloro-4-oxo-4,5-dihydropyrazolo[1,5-a]pyrazin-3-yl)benzonitrile (100 mg, 0.369 mmol) in DMF (3 mL) was added NaH (44 mg, 1.11 mmol, 60% dispersion in mineral oil) at 0° C. The mixture was stirred at 0° C. for 0.5 hour, then Mel (105 mg, 0.739 mmol) was added to the reaction mixture at 0° C. The resulting mixture was stirred at 25° C. for 2 hours. The reaction mixture was quenched by addition water (5 mL) and concentrated. The residue was purified by Combi Flash (0% to 30% EtOAc in PE) to give 4-(6-chloro-5-methyl-4-oxo-4,5-dihydropyrazolo[1,5-a]pyrazin-3-yl)benzonitrile (40 mg, yield: 38%) as a white solid.

¹H NMR (400 MHz, DMSO-d₆) δ 3.55 (3H, s), 7.89 (2H, d, J=8.8 Hz), 8.05 (2H, d, J=8.8 Hz), 8.32 (1H, s), 8.36 (1H, s).

### Step 3. Synthesis of 4-(6-(4-fluorophenyl)-5-methyl-4-oxo-4,5-dihydropyrazolo[1,5-a]pyrazin-3-yl)benzonitrile

A mixture of 4-(6-chloro-5-methyl-4-oxo-4,5-dihydropyrazolo[1,5-a]pyrazin-3-yl)benzonitrile (40 mg, 0.14 mmol), 4-fluorophenylboronic acid (30 mg, 0.21 mmol), Xphos-Pd-G₃ (12 mg, 0.014 mmol) and K₂CO₃ (39 mg, 0.28 mmol) in 1,4-dioxane (2 mL) and H₂O (0.4 mL) was degassed and purged with N₂ for 3 times, and then the mixture was stirred at 100° C. for 16 hours under N₂ atmosphere. The reaction mixture was filtered and the filtrate was concentrated. The residue was purified by prep-HPLC (0.225% FA as an additive) then lyophilized to give the title compound (11.8 mg, yield: 24%) as a white solid.

¹H NMR (400 MHz, DMSO-d₆) δ 3.21 (3H, s), 7.39 (2H, t, J=8.8 Hz), 7.63-7.70 (2H, m), 7.82 (1H, s), 7.90 (2H, d, J=8.8 Hz), 8.12 (2H, d, J=8.4 Hz), 8.38 (1H, s).

The following compounds were synthesized analogously to Example 16

| Example | | | |
|---|---|---|---|
| No. | Structure | Name | ¹H NMR (400 MHz) |
| 32 | 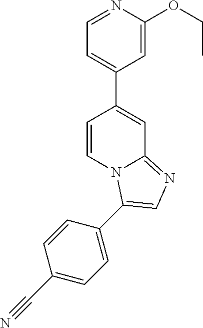 [SMILES: CCOc1cc(-c2ccn3c(-c4ccc(C#N)cc4)cnc3c2)ccn1] | 4-(7-(2- ethoxypyridin-4- yl)imidazo[1,2- a]pyridin-3- yl)benzonitrile | CDCl₃; δ1.44 (3H, t, J = 6.8 Hz), 4.43 (2H, q, J = 7.2 Hz), 7.01 (1H, s), 7.13-7.21 (2H, m), 7.71- 7.77 (2H, m), 7.81-7.90 (3H, m), 8.00 (1H, s), 8.26 (1H, d, J = 5.6 Hz), 8.45 (1H, d, J = 7.2 Hz). |
| 38 | 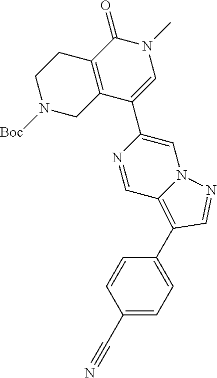 [SMILES: Cn1cc(-c2cn3ncc(-c4ccc(C#N)cc4)c3cn2)c2c(c1=O)CCN(C(=O)OC(C)(C)C)C2] | tert-butyl 8-(3-(4- cyanophenyl)pyrazolo [1,5-a]pyrazin-6-yl)- 6-methyl-5-oxo- 3,4,5,6-tetrahydro- 2,6-naphthyridine- 2(1H)-carboxylate | DMSO-d₆; δ 1.37 (9H, s), 2.53- 2.55 (2H, m), 3.50 (3H, s), 3.55- 3.57 (2H, m), 4.51 (2H, s), 7.91 (1H, s), 7.95 (2H, d, J = 8.0 Hz), 8.10 (2H, d, J = 8.0 Hz), 8.78 (1H, s), 9.00 (1H, d, J = 1.2 Hz), 9.60 (1H, s). |

### Example 18

### N-(5-(4-cyano-3-hydroxyphenyl)thiazol-2-yl)-1-methylpiperidine-4-carboxamide

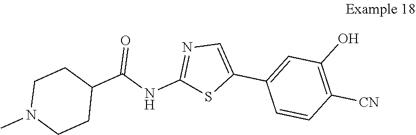 [SMILES: CN1CCC(C(=O)Nc2ncc(-c3ccc(C#N)c(O)c3)s2)CC1]

### Step 1. Synthesis of 2-hydroxy-4-(4,4,5,5-tetramethyl-1,3,2-dioxaborolan-2-yl)benzaldehyde

A mixture of 4-bromo-2-hydroxybenzaldehyde (1.00 g, 4.97 mmol), Bis-Pin (1.64 g, 6.47 mmol), KOAc (1.46 g, 14.9 mmol) and Pd(dppf)Cl₂ (364 mg, 0.497 mmol) in dioxane (20 mL) was degassed and purged with N₂ for 3 times, and then the mixture was stirred at 90° C. for 12 hours under N₂ atmosphere. The reaction mixture was suspended in CH₃OH (50 mL) and filtered. The filtrate was concentrated and the residue was purified by silica gel column (000 to 500 EtOAc in PE) to give 2-hydroxy-4-(4,4,5,5-tetramethyl-1,3,2-dioxaborolan-2-yl)benzaldehyde (700 mg, yield: 570%) as a yellow solid.

¹H NMR (400 MHz, DMSO-d₆) δ 1.30 (12H, s), 7.20 (1H, d, J=7.6 Hz), 7.30 (1H, s), 7.64 (1H, d, J=7.6 Hz), 10.33 (1H, s), 10.66 (1H, brs).

### Step 2. Synthesis of N-(5-(4-formyl-3-hydroxyphenyl)thiazol-2-yl)-1-methylpiperidine-4-carboxamide

A mixture of 2-hydroxy-4-(4,4,5,5-tetramethyl-1,3,2-dioxaborolan-2-yl)benzaldehyde (700 mg, 2.30 mmol), N-(5-bromothiazol-2-yl)-1-methylpiperidine-4-carboxamide (685 mg, 2.76 mmol), Na₂CO₃ (732 mg, 6.90 mmol) and Pd(dtbpf)Cl₂ (150 mg, 0.230 mmol) in dioxane (10 mL) and H₂O (1 mL) was degassed and purged with N₂ for 3 times, then the mixture was stirred at 90° C. for 12 hours under N₂ atmosphere. The reaction mixture was suspended in CH₃OH (50 mL) and filtered. The filtrate was concentrated and the residue was purified by silica gel column (0% to 10% MeOH in DCM) to give N-(5-(4-formyl-3-hydroxyphenyl)thiazol-2-yl)-1-methylpiperidine-4-carboxamide (500 mg, yield: 63%) as a yellow solid.

### Step 3. Synthesis of (E)-N-(5-(3-hydroxy-4-((hydroxyimino)methyl)phenyl)thiazol-2-yl)-1-methylpiperidine-4-carboxamide

To a solution of N-(5-(4-formyl-3-hydroxyphenyl)thiazol-2-yl)-1-methylpiperidine-4-carboxamide (500 mg, 1.45 mmol), NH₂OH·HCl (121 mg, 1.74 mmol) in EtOH (5 mL) was added NaOAc (142 mg, 1.74 mmol) and H₂O (1 mL). The mixture was stirred at 25° C. for 12 hours. The reaction mixture was concentrated under reduced pressure to remove EtOH and filtered. The solid was washed with water (10 mL×2) and dried to give (E)-N-(5-(3-hydroxy-4-((hydroxyimino)methyl)phenyl)thiazol-2-yl)-1-methylpiperidine-4-carboxamide (300 mg, yield: 57%) as a yellow solid.

¹H NMR (400 MHz, DMSO-d₆) δ 1.61-1.72 (2H, m), 1.77-1.83 (2H, m), 1.88-2.01 (2H, m), 2.21 (3H, s), 2.43-2.48 (1H, m), 2.81-2.90 (2H, m), 7.08 (1H, d, J=1.6 Hz), 7.14 (1H, dd, J=8.0, 1.6 Hz), 7.52 (1H, d, J=8.4 Hz), 7.88 (1H, s), 8.32 (1H, s), 10.28 (1H, s), 11.37 (1H, brs), 12.22 (1H, brs).

### Step 4. Synthesis of N-(5-(4-cyano-3-hydroxyphenyl)thiazol-2-yl)-1-methylpiperidine-4-carboxamide

To a solution of PPh₃ (546 mg, 2.08 mmol) in THF (4 mL) was added DIAD (421 mg, 2.08 mmol). The mixture was stirred at 25° C. for 0.5 hour. Then (E)-N-(5-(3-hydroxy-4-((hydroxyimino)methyl)phenyl)thiazol-2-yl)-1-methylpiperidine-4-carboxamide (250 mg, 0.694 mmol) was added to the mixture and stirred at 25° C. for 1 hour. The reaction mixture was concentrated and the residue was triturated with EtOAc (5 mL), then further purified by prep-HPLC (Method C; 0.225% FA as an additive) and lyophilized to afford the title compound (73.56 mg, yield: 27%, FA salt) as a yellow solid.

¹H NMR (400 MHz, DMSO-d₆) δ 1.57-1.74 (2H, m), 1.76-1.88 (2H, m), 1.93-2.06 (2H, m), 2.23 (3H, s), 2.52-2.55 (1H, m), 2.86-2.91 (2H, m), 7.12 (1H, s), 7.23 (1H, dd, J=8.4, 1.6 Hz), 7.61 (1H, d, J=8.0 Hz), 7.97 (1H, s), 8.17 (1H, s), 12.50 (1H, brs).

### Example 20

### ethyl 4-(6-(1-methylpiperidine-4-carboxamido)-1H-pyrrolo[2,3-b]pyridin-3-yl)benzoate

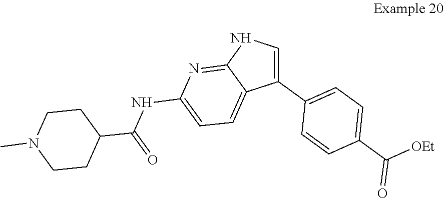 [SMILES: CCOC(=O)c1ccc(-c2c[nH]c3nc(NC(=O)C4CCN(C)CC4)ccc23)cc1]

### Step 1. Synthesis of ethyl 4-(6-chloro-1-tosyl-1H-pyrrolo[2,3-b]pyridin-3-yl)benzoate

To a solution of 6-chloro-3-iodo-1-tosyl-1H-pyrrolo[2,3-b]pyridine (2.00 g, 4.62 mmol) and (4-(ethoxycarbonyl)phenyl)boronic acid (986 mg, 5.08 mmol) in 1, 4-dioxane (25 mL) and H₂O (5 mL) was added Pd(dppf)Cl₂ (507 mg, 0.693 mmol) and Na₂CO₃ (1.47 g, 13.9 mmol) under N₂ atmosphere, the mixture was stirred at 90° C. for 12 hours under N₂ atmosphere. The reaction mixture was concentrated and the residue was purified by flash silica gel chromatography (ISCO®; 20 g SepaFlash® Silica Flash Column, Eluent of 16˜50% Ethyl acetate/Petroleum ether gradient @35 mL/min) to give ethyl 4-(6-chloro-1-tosyl-1H-pyrrolo[2,3-b]pyridin-3-yl)benzoate (2.60 g, yield: 99%) as a brown solid.

### Step 2. Synthesis of ethyl 4-(6-((tert-butoxycarbonyl)amino)-1-tosyl-1H-pyrrolo[2,3-b]pyridin-3-yl)benzoate

To a solution of ethyl 4-(6-chloro-1-tosyl-1H-pyrrolo[2,3-b]pyridin-3-yl)benzoate (650 mg, 1.43 mmol) and BocNH₂ (418 mg, 3.57 mmol) in 1, 4-dioxane (15 mL) was added Pd₂(dba)₃ (131 mg, 143 mmol), Cs₂CO₃ (1.40 g, 4.29 mmol) and XPhos (136 mg, 0.286 mol) under N₂ atmosphere, the mixture was stirred at 90° C. for 12 hours under N₂ atmosphere. The reaction mixture was filtered and the filtrate was concentrated. The residue was diluted with H₂O (50 mL) and extracted with EtOAc (50 mL×2). The combined organic layer was dried over anhydrous Na₂SO₄, filtered and concentrated to give ethyl 4-(6-((tert-butoxycarbonyl)amino)-1-tosyl-1H-pyrrolo[2,3-b]pyridin-3-yl)benzoate (1.10 g, crude) as brown gum, which was used into the next step without further purification.

### Step 3. Synthesis of ethyl 4-(6-amino-1-tosyl-1H-pyrrolo[2,3-b]pyridin-3-yl)benzoate

To a solution of ethyl 4-(6-((tert-butoxycarbonyl)amino)-1-tosyl-1H-pyrrolo[2,3-b]pyridin-3-yl)benzoate (1.10 g, crude) in DCM (10 mL) was added TFA (10 mL), the mixture was stirred at 25° C. for 2 hours. The reaction mixture was concentrated and the residue was basified with saturated aqueous NaHCO₃ to pH=8 and diluted into H₂O (40 mL), extracted with EtOAc (40 mL×2). The combined organic layer was dried over anhydrous Na₂SO₄, filtered and concentrated. The residue was purified by flash silica gel chromatography (ISCO®; 12 g SepaFlash® Silica Flash Column, Eluent of ˜40% Ethyl acetate/Petroleum ether gradient @25 mL/min) to give ethyl 4-(6-amino-1-tosyl-1H-pyrrolo[2,3-b]pyridin-3-yl)benzoate (430 mg, yield: 69% for two steps) as a brown solid.

### Step 4. Synthesis of ethyl 4-(6-amino-1H-pyrrolo[2,3-b]pyridin-3-yl)benzoate

To a solution of ethyl 4-(6-amino-1-tosyl-1H-pyrrolo[2,3-b]pyridin-3-yl)benzoate (430 mg, 0.987 mmol) in THF (8 mL) was added TBAF (2.96 mL, 2.96 mmol, 1.0M in THF), the mixture was stirred at 60° C. for 12 hours. The reaction mixture was concentrated and the residue was diluted with EtOAc (50 mL), washed with saturated aqueous NH₄Cl (40 mL×3), brine (50 mL), dried over anhydrous Na₂SO₄, filtered and concentrated. The residue was purified by flash silica gel chromatography (ISCO®; 12 g SepaFlash® Silica Flash Column, Eluent of ˜80% Ethyl acetate/Petroleum ether gradient @30 mL/min) to give ethyl 4-(6-amino-1H-pyrrolo[2,3-b]pyridin-3-yl)benzoate (200 mg, yield: 70%) as a brown solid.

¹H NMR (400 MHz, DMSO-d₆) δ 1.33 (3H, t, J=7.2 Hz), 4.31 (2H, q, J=6.8 Hz), 5.76 (2H, brs), 6.37 (1H, d, J=8.8 Hz), 7.53 (1H, d, J=2.4 Hz), 7.79 (2H, d, J=8.4 Hz), 7.90-8.05 (3H, m), 11.28 (1H, brs).

### Step 5. Synthesis of ethyl 4-(6-(1-methylpiperidine-4-carboxamido)-1H-pyrrolo[2,3-b]pyridin-3-yl)benzoate

To a solution of ethyl 4-(6-amino-1H-pyrrolo[2,3-b]pyridin-3-yl)benzoate (200 mg, 0.711 mmol), 1-methylpiperidine-4-carboxylic acid (305 mg, 2.13 mmol) and TEA (288 mg, 2.84 mmol) in pyridine (5 mL) was added EDCI (545 mg, 2.84 mmol), the mixture was stirred at 90° C. for 12 hours. The reaction mixture was concentrated. The residue was diluted into H₂O (40 mL), extracted with EtOAc (40 mL×3). The combined organic layer was dried over anhydrous Na₂SO₄, filtered and concentrated. The residue was purified by flash silica gel chromatography (ISCO®; 12 g SepaFlash® Silica Flash Column, Eluent of ˜18% MeOH/DCM gradient @30 mL/min) to give the title compound (220 mg, yield: 73%) as a yellow solid.

¹H NMR (400 MHz, DMSO-d₆) δ 1.34 (3H, t, J=7.2 Hz), 1.61-1.83 (4H, m), 1.95-2.06 (2H, m), 2.23 (3H, s) 2.51-2.56 (1H, m), 2.85-2.93 (2H, m), 4.32 (2H, q, J=6.8 Hz), 7.85-7.91 (2H, m), 7.95 (1H, d, J=2.8 Hz), 7.96-8.02 (3H, m), 8.21 (1H, s), 8.35 (1H, d, J=8.8 Hz), 10.32 (1H, brs), 11.86 (1H, brs).

### Example 21

### 4-(6-(1-methylpiperidine-4-carboxamido)-1H-pyrrolo[2,3-b]pyridin-3-yl)benzoic acid

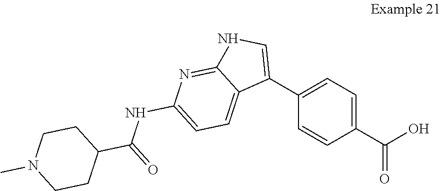 [SMILES: CN1CCC(C(=O)Nc2ccc3c(-c4ccc(C(=O)O)cc4)c[nH]c3n2)CC1]

To a solution of Example 21 (130 mg, 0.32 mmol) in MeOH (3 mL), THF (3 mL) and H₂O (3 mL) was added LiOH·H₂O (48 mg, 1.2 mmol), the mixture was stirred at 60° C. for 2 hours. The reaction mixture was acidified with 1N aqueous HCl to pH=6 and concentrated. The residue was purified by prep-HPLC (0.04% NH₃H₂O+10 mM NH₄HCO₃ as an additive) and lyophilized to give the title compound (23 mg, yield: 19%) as a light yellow solid.

¹H NMR (400 MHz, DMSO-d₆) δ 1.59-1.82 (4H, m), 1.85-1.97 (2H, m), 2.12-2.23 (4H, m), 2.80-2.87 (2H, m), 7.81-7.88 (2H, m), 7.92 (1H, d, J=2.4 Hz), 7.94-8.02 (3H, m), 8.34 (1H, d, J=8.8 Hz), 10.29 (1H, brs), 11.82 (1H, brs).

The following compounds were synthesized analogously to Example 21

| Example | | | |
|---|---|---|---|
| No. | Structure | Name | ¹H NMR (400 MHz) |
| 26 | 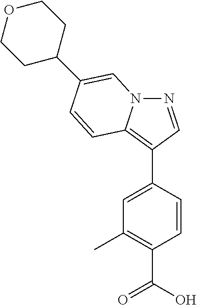 [SMILES: Cc1cc(-c2cnn3cc(C4CCOCC4)ccc23)ccc1C(=O)O] | 2-methyl-4-(6- (tetrahydro-2H- pyran-4- yl)pyrazolo[1,5- a]pyridin-3- yl)benzoic acid | DMSO-d₆; δ 1.72-1.85 (4H, m), 2.61(3H, s), 2.87-2.91 (1H, m), 3.42-3.50 (2H, m), 3.90-4.05 (2H, m), 7.36 (1H, s), 7.57-7.67 (2H, m), 7.92 (1H, d, J = 8.0 Hz), 8.02 (1H, d, J = 8.0 Hz), 8.43 (1H, s), 8.59 (1H, s), 12.70 (1H, brs). |
| 31 | 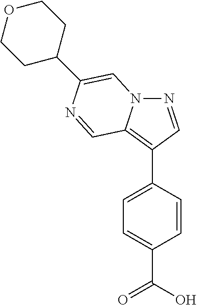 [SMILES: O=C(O)c1ccc(-c2cnn3cc(C4CCOCC4)ncc23)cc1] | 4-(6-(tetrahydro-2H- pyran-4- yl)pyrazolo[1,5- a]pyrazin-3- yl)benzoic acid | DMSO-d₆; δ 1.79-1.92 (4H, m), 2.98-3.05 (1H, m), 3.50-3.52 (2H, m), 3.95-4.03 (2H, m), 7.96 (2H, d, J = 8.0 Hz), 8.03 (2H, d, J = 8.4 Hz), 8.62 (1H, s), 8.69 (1H, s), 9.53 (1H, s). |
| 35 | 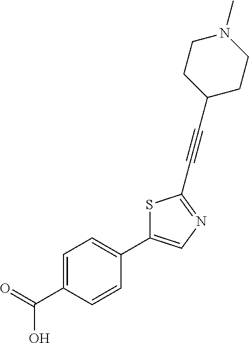 [SMILES: CN1CCC(C#Cc2ncc(-c3ccc(C(=O)O)cc3)s2)CC1] | 4-(2-((1- methylpiperidin-4- yl)ethynyl)thiazol-5- yl)benzoic acid | DMSO-d₆; δ 1.65-1.75 (2H, m), 1.90-1.95 (2H, m), 2.20-2.30 (5H, m), 2.60-2.70 (3H, m), 7.81 (2H, d, J = 8.4 Hz), 7.99 (2H, d, J = 8.4 Hz). 8.17 (2H, s), 8.40 (1H, s). |
| 51 | 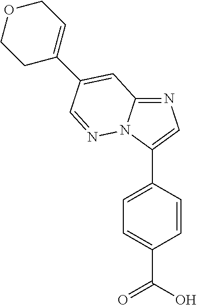 [SMILES: O=C(O)c1ccc(-c2cnc3cc(C4=CCOCC4)cnn23)cc1] | 4-(7-(3,6-dihydro- 2H-pyran-4- yl)imidazo[1,2- b]pyridazin-3- yl)benzoic acid | DMSO-d₆; δ 2.54-2.56 (2H, m), 3.88 (2H, t, J = 5.6 Hz), 4.25- 4.30 (2H, m), 6.75-6.78 (1H, m), 8.07 (2H, d, J = 8.4 Hz), 8.13 (1H, s), 8.34 (2H, d, J = 8.4 Hz), 8.41 (1H, s), 9.03 (1H, d, J = 2.0 Hz), 13.01 (1H, brs). |
| 53 | 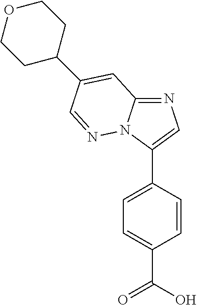 [SMILES: O=C(O)c1ccc(-c2cnc3cc(C4CCOCC4)cnn23)cc1] | 4-(7-(tetrahydro-2H- pyran-4- yl)imidazo[1,2- b]pyridazin-3- yl)benzoic acid | DMSO-d₆; δ 1.74-1.87 (4H, m), 2.95-3.04 (1H, m), 3.40-3.51 (2H, m), 3.95-4.05 (2H, m), 8.02-8.10 (3H, m), 8.29-8.35 (2H, m), 8.38 (1H, s), 8.75 (1H, d, J = 2.0 Hz). |

### Example 22

### tert-butyl 4-(6-(tetrahydro-2H-pyran-4-yl)pyrazolo[1,5-a]pyridin-3-yl)benzoate

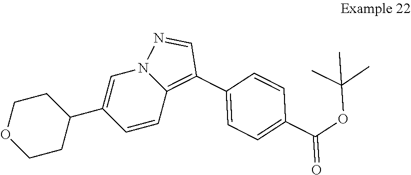 [SMILES: CC(C)(C)OC(=O)c1ccc(-c2cnn3cc(C4CCOCC4)ccc23)cc1]

### Step 1. Synthesis of tert-butyl 4-(6-bromopyrazolo[1,5-a]pyridin-3-yl)benzoate

A mixture of 6-bromo-3-iodopyrazolo[1,5-a]pyridine (500 mg, 1.55 mmol), tert-butyl 4-(4,4,5,5-tetramethyl-1,3,2-dioxaborolan-2-yl)benzoate (565 mg, 1.86 mmol), K₂CO₃ (427 mg, 3.10 mmol) and Pd(dppf)Cl₂ (113 mg, 0.154 mmol) in dioxane (10 mL) and H₂O (1 mL) was degassed and purged with N₂ for 3 times, then the mixture was stirred at 90° C. for 2 hours under N₂ atmosphere. The reaction mixture was concentrated and the residue was purified by Combi Flash (0% to 20% EtOAc in PE) to give tert-butyl 4-(6-bromopyrazolo[1,5-a]pyridin-3-yl)benzoate (315 mg, yield: 54%) as a white solid.

### Step 2. Synthesis of tert-butyl 4-(6-(3,6-dihydro-2H-pyran-4-yl)pyrazolo[1,5-a]pyridin-3-yl)benzoate

A mixture of tert-butyl 4-(6-bromopyrazolo[1,5-a]pyridin-3-yl)benzoate (315 mg, 0.844 mmol), 2-(3,6-dihydro-2H-pyran-4-yl)-4,4,5,5-tetramethyl-1,3,2-dioxaborolane (212 mg, 1.01 mmol), Pd(dppf)Cl₂ (62 mg, 0.084 mmol) and K₂CO₃ (233 mg, 1.69 mmol) in dioxane (10 mL) and H₂O (1 mL) was degassed and purged with N₂ for 3 times, and then the mixture was stirred at 90° C. for 16 hours under N₂ atmosphere. The reaction mixture was concentrated and the residue was purified by Combi Flash (0% to 20% EtOAc in PE) to give tert-butyl 4-(6-(3,6-dihydro-2H-pyran-4-yl)pyrazolo[1,5-a]pyridin-3-yl)benzoate (200 mg, yield: 62%) as a white solid.

¹H NMR (400 MHz, DMSO-d₆) δ 1.54-1.60 (9H, m), 3.29-3.32 (2H, m), 3.80-3.89 (2H, m), 4.20-4.33 (2H, m), 6.42-6.53 (1H, m), 7.66 (1H, dd, J=8.0, 1.6 Hz), 7.85 (2H, d, J=8.8 Hz), 7.96 (2H, d, J=8.4 Hz), 8.00-8.06 (1H, m), 8.51 (1H, s), 8.71-8.75 (1H, m).

### Step 3. Synthesis of tert-butyl 4-(6-(tetrahydro-2H-pyran-4-yl)pyrazolo[1,5-a]pyridin-3-yl)benzoate

A mixture of tert-butyl 4-(6-(3,6-dihydro-2H-pyran-4-yl)pyrazolo[1,5-a]pyridin-3-yl)benzoate (150 mg, 0.398 mmol) and Pd/C (100 mg, 0.398 umol, 10% purity) in absolute MeOH (5 mL) was degassed and purged with H₂ for 3 times, and then the mixture was stirred at 25° C. for 12 hours under H₂ atmosphere (15 psi). The reaction mixture was filtered through a pad of celite and the filtrate was concentrated to give the title compound (120 mg, yield: 79%) as a black solid.

The following compounds were synthesized analogously to Example 22

| Example | | | |
|---|---|---|---|
| No. | Structure | Name | ¹H NMR (400 MHz) |
| 24 | 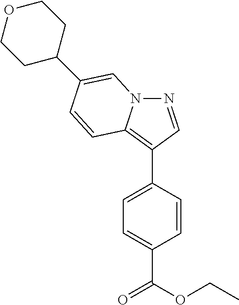 [SMILES: CCOC(=O)c1ccc(-c2cnn3cc(C4CCOCC4)ccc23)cc1] | ethyl 4-(6- (tetrahydro-2H- pyran-4- yl)pyrazolo[1,5- a]pyridin-3- yl)benzoate | DMSO-d₆; δ 1.33 (3H, t, J = 7.2 Hz), 1.67-1.83 (4H, m), 2.83- 2.95 (1H, m), 3.44-3.50 (2H, m), 3.95-4.00 (2H, m), 4.32 (2H, q, J = 7.2 Hz), 7.43 (1H, dd, J = 9.2, 1.6 Hz), 7.82-7.90 (2H, m), 7.97-8.07 (3H, m), 8.48 (1H, s), 8.61 (1H, s). |
| 25 | 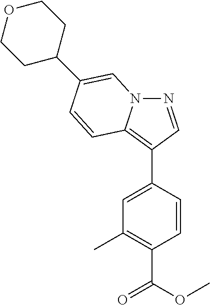 [SMILES: COC(=O)c1ccc(-c2cnn3cc(C4CCOCC4)ccc23)cc1C] | methyl 2-methyl-4- (6-(tetrahydro-2H- pyran-4- yl)pyrazolo[1,5- a]pyridin-3- yl)benzoate | — |
| 27 | 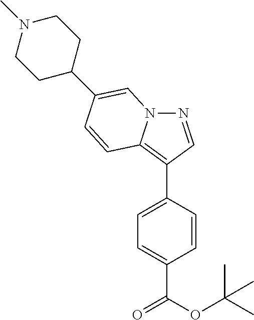 [SMILES: CN1CCC(c2ccc3c(-c4ccc(C(=O)OC(C)(C)C)cc4)cnn3c2)CC1] | tert-butyl 4-(6-(1- methylpiperidin-4- yl)pyrazolo[1,5- a]pyridin-3- yl)benzoate | — |
| 44 | 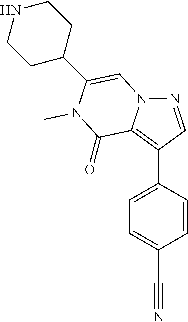 [SMILES: Cn1c(C2CCNCC2)cn2ncc(-c3ccc(C#N)cc3)c2c1=O] | 4-(5-methyl-4-oxo-6- (piperidin-4-yl)-4,5- dihydropyrazolo[1,5- a]pyrazin-3- yl)benzonitrile | CD₃OD) δ 1.80-1.95 (2H, m), 2.25-2.37 (2H, m), 3.17-3.31 (3H, m), 3.54-3.61 (2H, m), 3.64 (3H, s), 7.59 (1H, s), 7.77 (2H, d, J = 8.4 Hz), 8.04 (2H, d, J = 8.8 Hz), 8.16 (1H, s). |

### Example 23

### 4-(6-(tetrahydro-2H-pyran-4-yl)pyrazolo[1,5-a]pyridin-3-yl)benzoic acid

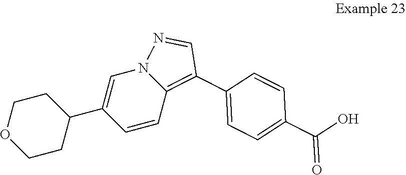 [SMILES: O=C(O)c1ccc(-c2cnn3cc(C4CCOCC4)ccc23)cc1]

To a solution of Example 22 (100 mg, 0.264 mmol) in anhydrous DCM (5 mL) was added TFA (2.62 g, 22.9 mmol). The mixture was stirred at 25° C. for 2 hours. The reaction mixture was concentrated. The residue was purified by pre-HPLC (0.225% FA as an additive) and lyophilized to give the title compound (28.91 mg, yield: 33=) as a white solid.

¹HNMR (400 MHz, DMSO-d₆) δ 1.68-1.85 (4H, m), 2.82-2.96 (1H, m), 3.42-3.50 (2H, m), 3.91-4.05 (2H, m), 7.32-7.47 (1H, m), 7.76-7.89 (2H, m), 7.97-8.01 (2H, m), 8.01-8.06 (1H, m), 8.39-8.51 (1H, m), 8.54-8.67 (1H, m).

The following compounds were synthesized analogously to Example 23

| Example | | | |
|---|---|---|---|
| No. | Structure | Name | ¹H NMR (400 MHz) |
| 28 | 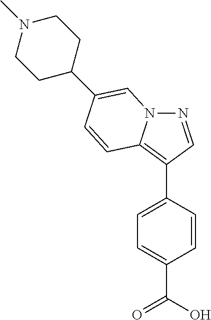 [SMILES: CN1CCC(c2ccc3c(-c4ccc(C(=O)O)cc4)cnn3c2)CC1] | 4-(6-(1- methylpiperidin-4- yl)pyrazolo[1,5- a]pyridin-3- yl)benzoic acid | DMSO-d₆; δ 1.67-1.87 (4H, m), 1.94-2.03 (2H, m), 2.21 (3H, s), 2.54-2.63 (1H, m), 2.87-2.91 (2H, m), 7.37 (1H, d, J = 9.2 Hz), 7.76 (2H, d, J = 8.4 Hz), 7.93-8.03 (3H, m), 8.42 (1H, s), 8.58 (1H, s). |
| 34 | 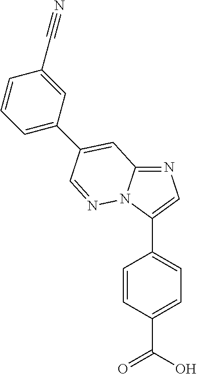 [SMILES: N#Cc1cccc(-c2cnn3c(-c4ccc(C(=O)O)cc4)cnc3c2)c1] | 4-(7-(3- cyanophenyl)imidazo [1,2-b]pyridazin-3- yl)benzoic acid | DMSO-d₆; δ 7.77 (1H, t, J = 8.0 Hz), 7.95 (1H, d, J = 8.0 Hz), 8.06-8.13 (2H, m), 8.33 (1H, d, J = 8.0 Hz), 8.35-8.41 (2H, m), 8.48-8.55 (2H, m), 8.75 (1H, d, J = 2.4 Hz), 9.23 (1H, d, J = 2.4 Hz). |
| 37 | 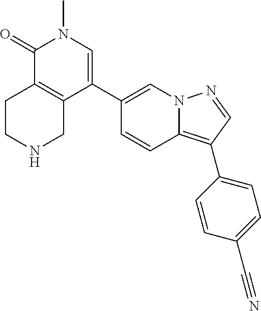 [SMILES: Cn1cc(-c2ccc3c(-c4ccc(C#N)cc4)cnn3c2)c2c(c1=O)CCNC2] | 4-(6-(2-methyl-1- oxo-1,2,5,6,7,8- hexahydro-2,6- naphthyridin-4- yl)pyrazolo[1,5- a]pyridin-3- yl)benzonitrile | DMSO-d₆; δ 2.39-2.46 (2H, m), 2.92-2.97 (2H, m), 3.47 (3H, s), 3.61 (2H, s), 7.42 (1H, dd, J = 9.2, 1.6 Hz), 7.66 (1H, s), 7.89 (2H, d, J = 8.4 Hz), 7.95 (2H, d, J = 8.8 Hz), 8.10 (1H, d, J = 9.2 Hz), 8.61 (1H, s), 8.77 (1H, s). |
| 39 | 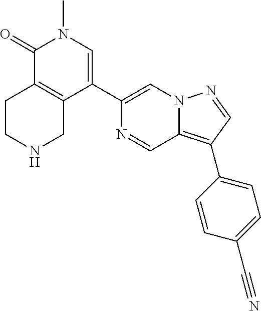 [SMILES: Cn1cc(-c2cn3ncc(-c4ccc(C#N)cc4)c3cn2)c2c(c1=O)CCNC2] | 4-(6-(2-methyl-1- oxo-1,2,5,6,7,8- hexahydro-2,6- naphthyridin-4- yl)pyrazolo[1,5- a]pyrazin-3- yl)benzonitrile | DMSO-d₆; δ 2.70-2.73 (2H, m), 3.41-3.43 (2H, m), 3.54 (3H, s), 4.28-4.30 (2H, m), 7.93 (2H, d, J = 8.0 Hz), 8.06 (1H, s), 8.09 (2H, d, J = 8.0 Hz), 8.80 (1H, s), 9.01 (1H, brs), 9.08 (1H, d, J = 1.2 Hz), 9.60 (1H, d, J = 1.6 Hz). |
| 42 | 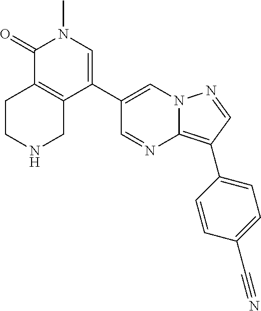 [SMILES: Cn1cc(-c2cnc3c(-c4ccc(C#N)cc4)cnn3c2)c2c(c1=O)CCNC2] | 4-(6-(2-methyl-1- oxo-1,2,5,6,7,8- hexahydro-2,6- naphthyridin-4- yl)pyrazolo[1,5- a]pyrimidin-3- yl)benzonitrile | — |

### Example 29

### 4-(6-(4,5,6,7-tetrahydrooxazolo[4,5-c]pyridin-2-yl)pyrazolo[1,5-a]pyridin-3-yl)benzoic acid

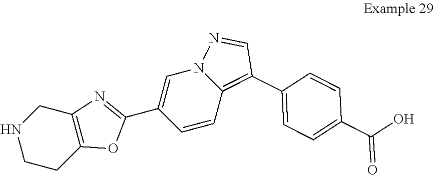 [SMILES: O=C(O)c1ccc(-c2cnn3cc(-c4nc5c(o4)CCNC5)ccc23)cc1]

### Step 1. Synthesis of tert-butyl 2-(3-(4-(tert-butoxycarbonyl)phenyl)pyrazolo[1,5-a]pyridin-6-yl)-6,7-dihydrooxazolo[4,5-c]pyridine-5(4H)-carboxylate

A mixture of Int-7 (110 mg, 0.490 mmol), tert-butyl 4-(6-bromnopyrazolo[1,5-a]pyridin-3-yl)benzoate (201 mg, 0.540 mmol), Pd(OAc)₂ (11 mg, 0.049 mmol), Cs₂CO₃ (320 mg, 0.981 mmol) and t-Bu₃PHBF₄ (28 mg, 0.098 mmol) in DMF (5 mL) was degassed and purged with N₂ for three times, the mixture was stirred at 120° C. for 3.5 hours under N₂ atmosphere. The reaction mixture was concentrated and the residue was purified by flash silica gel chromatography (ISCO®; 4 g SepaFlash® Silica Flash Column, Eluent of ˜19% Ethyl acetate/Petroleum ether gradient @35 mL/min) to give tert-butyl 2-(3-(4-(tert-butoxycarbonyl)phenyl)pyrazolo[1,5-a]pyridin-6-yl)-6,7-dihydrooxazolo[4,5-c]pyridine-5(4H)-carboxylate (120 mg, yield: 47%) as a yellow solid.

¹H NMR (400 MHz, CDCl₃) δ 1.51 (9H, s), 1.63 (9H, s), 2.83-2.92 (2H, m), 3.79-3.90 (2H, m), 4.51 (2H, s), 7.66 (2H, d, J=8.4 Hz), 7.81-7.86 (1H, m), 7.88-7.93 (1H, m), 8.09 (2H, d, J=8.8 Hz), 8.28 (1H, s), 9.14 (1H, s).

### Step 2. Synthesis of 4-(6-(4,5,6,7-tetrahydrooxazolo[4,5-c]pyridin-2-yl)pyrazolo[1,5-a]pyridin-3-yl)benzoic acid

To a solution of tert-butyl 2-(3-(4-(tert-butoxycarbonyl)phenyl)pyrazolo[1,5-a]pyridin-6-yl)-6,7-dihydrooxazolo[4,5-c]pyridine-5(4H)-carboxylate (85 mg, 0.16 mmol) in DCM (3 mL) was added TFA (1.5 mL) at 0° C., the mixture was stirred at 0° C. for 5 hours. The reaction mixture was concentrated and the residue was purified by prep-HPLC (0.1% TFA as an additive), then lyophilized to give (30 mg, yield: 39% the title compound, TFA salt) as a white solid.

¹H NMR (400 MHz, DMSO-d₆) δ 3.01-3.14 (2H, m), 3.49-3.62 (2H, m), 4.19-4.34 (2H, m), 7.84 (1H, dd, J=9.6, 1.2 Hz), 7.88 (2H, d, J=8.4 Hz), 8.03 (2H, d, J=8.0 Hz), 8.23 (1H, d, J=9.2 Hz), 8.68 (1H, s), 9.25-9.35 (3H, m), 12.65 (1H, brs).

### Example 30

### ethyl 4-(6-(tetrahydro-2H-pyran-4-yl)pyrazolo[1,5-a]pyrazin-3-yl)benzoate

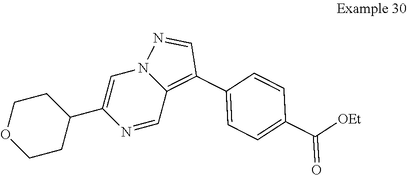 [SMILES: CCOC(=O)c1ccc(-c2cnn3cc(C4CCOCC4)ncc23)cc1]

### Step 1. Synthesis of ethyl 4-(6-chloropyrazolo[1,5-a]pyrazin-3-yl)benzoate

A mixture of Int-8 (500 mg, 1.79 mmol), (4-ethoxycarbonylphenyl)boronic acid (521 mg, 2.68 mmol) and Pd(dppf)Cl₂ (131 mg, 0.179 mmol), Na₂CO₃ (569 mg, 5.37 mmol) in dioxane (15 mL) and H₂O (3 mL) was degassed and purged with N₂ for 3 times. Then the reaction mixture was stirred at 90° C. for 2 hours under N₂ atmosphere. The reaction mixture was diluted with water (50 mL), then extracted with EtOAc (50 mL×2). The combined organic layer was washed with brine (25 mL), dried over anhydrous Na₂SO₄, filtered and concentrated. The residue was purified by silica gel column (5% to 20% EtOAc in PE), then triturated with PE/EtOAc (20 mL, 5/1) to give ethyl 4-(6-chloropyrazolo[1,5-a]pyrazin-3-yl)benzoate (460 mg, yield: 85%) as a yellow solid.

### Step 2. Synthesis of ethyl 4-(6-(3,6-dihydro-2H-pyran-4-yl)pyrazolo[1,5-a]pyrazin-3-yl)benzoate

A mixture of ethyl 4-(6-chloropyrazolo[1,5-a]pyrazin-3-yl)benzoate (100 mg, 0.331 mmol), 2-(3,6-dihydro-2H-pyran-4-yl)-4,4,5,5-tetramethyl-1,3,2-dioxaborolane (84 mg, 0.40 mmol), XPhos-Pd-G₃ (28 mg, 0.033 mmol) and K₂CO₃ (92 mg, 0.66 mmol) in dioxane (4 mL) and H₂O (0.5 mL) was degassed and purged with N₂ for 3 times, and then the mixture was stirred at 90° C. for 16 hours under N₂ atmosphere. The reaction mixture was concentrated and the residue was purified by silica gel column (0% to 100% DCM in PE) to give ethyl 4-(6-(3,6-dihydro-2H-pyran-4-yl)pyrazolo[1,5-a]pyrazin-3-yl)benzoate (100 mg, yield: 86%) as a yellow solid.

¹H NMR (400 MHz, DMSO-d₆) δ 1.36 (3H, t, J=7.2 Hz), 2.53-2.60 (2H, m), 3.87 (2H, t, J=5.6 Hz), 4.29-4.39 (4H, m), 6.92-6.97 (1H, m), 8.01 (2H, d, J=8.8 Hz), 8.06 (2H, d, J=8.8 Hz), 8.67 (1H, s), 8.79 (1H, s), 9.54 (1H, s).

### Step 3. Synthesis of ethyl 4-(6-(tetrahydro-2H-pyran-4-yl)pyrazolo[1,5-a]pyrazin-3-yl)benzoate

To a solution of ethyl 4-(6-(3,6-dihydro-2H-pyran-4-yl)pyrazolo[1,5-a]pyrazin-3-yl)benzoate (100 mg, 0.286 mmol) in THF (3 mL) was added 10% Pd/C (100 mg) at 25° C. The mixture was degassed and purged with H₂ for 3 times, then hydrogenated (15 psi) at 25° C. for 3 hours. The reaction mixture was filtered and the solid was washed with THF (5 mL×3), the filtrate was concentrated to give the title compound (90 mg, crude) as a yellow solid.

### Example 33

### tert-butyl 4-(7-(3-cyanophenyl)imidazo[1,2-b]pyridazin-3-yl)benzoate

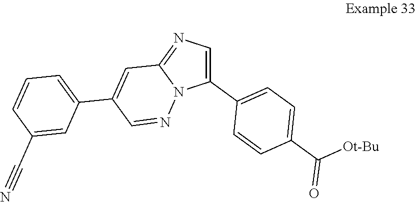 [SMILES: CC(C)(C)OC(=O)c1ccc(-c2cnc3cc(-c4cccc(C#N)c4)cnn23)cc1]

### Step 1. Synthesis of tert-butyl 4-(7-chloroimidazo[1,2-b]pyridazin-3-yl)benzoate

To a mixture of Int-10 (80 mg, 0.29 mmol), (4-(tert-butoxycarbonyl)phenyl)boronic acid (96 mg, 0.31 mmol), Pd(dppf)Cl₂ (21 mg, 0.029 mmol) and Na₂CO₃ (61 mg, 0.57 mmol) in 1, 4-dioxane (2 mL) and H₂O (0.2 mL) was degassed and purged with N₂ for 3 times, and then the mixture was stirred at 90° C. for 2 hours under N₂ atmosphere. The reaction mixture was concentrated and the residue was purified by silica gel column (0% to 20% EtOAc in PE) to give tert-butyl 4-(7-chloroimidazo[1,2-b]pyridazin-3-yl)benzoate (80 mg, yield: 67%) as a yellow solid.

¹H NMR (400 MHz, CD₃OD-d₄) δ 1.65 (9H, s), 8.07-8.12 (2H, m), 8.24-8.27 (2H, m), 8.26-8.30 (2H, m), 8.67 (1H, s).

### Step 2. Synthesis of tert-butyl 4-(7-(3-cyanophenyl)imidazo[1,2-b]pyridazin-3-yl)benzoate

To a mixture of tert-butyl 4-(7-chloroimidazo[1,2-b]pyridazin-3-yl)benzoate (70 mg, 0.21 mmol), (3-cyanophenyl)boronic acid (37 mg, 0.25 mmol), XPhos-Pd-G₃ (18 mg, 0.021 mmol) and K₂CO₃ (59 mg, 0.42 mmol) in 1, 4-dioxane (2 mL) and H₂O (0.2 mL) was degassed and purged with N₂ for 3 times, and then the mixture was stirred at 90° C. for 12 hours under N₂ atmosphere. The reaction mixture was concentrated and the residue was purified by silica gel column (0% to 50% EtOAc in PE) to give the title compound (50 mg, yield: 52%) as a yellow solid.

### Example 36

### tert-butyl 8-(3-(4-cyanophenyl)pyrazolo[1,5-a]pyridin-6-yl)-6-methyl-5-oxo-3,4,5,6-tetrahydro-2,6-naphthyridine-2(1H)-carboxylate

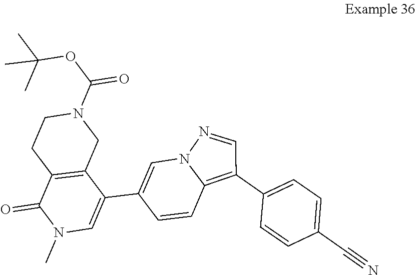 [SMILES: Cn1cc(-c2ccc3c(-c4ccc(C#N)cc4)cnn3c2)c2c(c1=O)CCN(C(=O)OC(C)(C)C)C2]

### Step 1. Synthesis of 6-bromo-3-iodopyrazolo[1,5-a]pyridine

To a solution of 6-bromopyrazolo[1,5-a]pyridine (2.50 g, 12.7 mmol) in DMF (30 mL) was added NIS (3.14 g, 14.0 mmol). The mixture was stirred at 20° C. for 1 hour. The reaction mixture was poured into H₂O (50 mL), the precipitate was filtered and dried to give 6-bromo-3-iodopyrazolo[1,5-a]pyridine (3.90 g, yield: 94%) as an off-white solid.

### Step 2. Synthesis of 4-(6-bromopyrazolo[1,5-a]pyridin-3-yl)benzonitrile

To a solution of 6-bromo-3-iodopyrazolo[1,5-a]pyridine (400 mg, 1.24 mmol), (4-cyanophenyl)boronic acid (197 mg, 1.34 mmol) in 1, 4-dioxane (8 mL) and H₂O (1.5 mL) was added Pd(dppf)Cl₂ (91 mg, 0.12 mmol) and Na₂CO₃ (263 mg, 2.48 mmol) under N₂ atmosphere, the mixture was stirred at 90° C. for 2 hours under N₂ atmosphere. The reaction mixture was concentrated and the residue was purified by flash silica gel chromatography (ISCO®; 12 g SepaFlash® Silica Flash Column, Eluent of ˜15% Ethyl acetate/Petroleum ether gradient @30 mL/min) to give 4-(6-bromopyrazolo[1,5-a]pyridin-3-yl)benzonitrile (240 mg, yield: 65%) as a yellow solid.

### Step 3. Synthesis of tert-butyl 8-(3-(4-cyanophenyl)pyrazolo[1,5-a]pyridin-6-yl)-6-methyl-5-oxo-3,4,5,6-tetrahydro-2,6-naphthyridine-2(1H)-carboxylate

To a solution of 4-(6-bromopyrazolo[1,5-a]pyridin-3-yl)benzonitrile (190 mg, 0.637 mmol) and Int-12 (311 mg, 0.797 mmol) in 1,4-dioxane (5 mL) and H₂O (1 mL) was added Pd₂(dba)₃ (58 mg, 0.064 mmol), Na₂CO₃ (135 mg, 1.27 mmol) and XPhos (61 mg, 0.13 mmol) under N₂ atmosphere, the mixture was stirred at 90° C. for 16 hours under N₂ atmosphere. The reaction mixture was concentrated and the residue was purified by flash silica gel chromatography (ISCO®; 12 g SepaFlash® Silica Flash Column, Eluent of ˜6% MeOH/DCM gradient @35 mL/min) to give the title compound (260 mg, yield: 85%) as a yellow gum.

### Example 40

### 4-(6-(2,6-dimethyl-1-oxo-1,2,5,6,7,8-hexahydro-2,6-naphthyridin-4-yl)pyrazolo[1,5-a]pyrazin-3-yl)benzonitrile

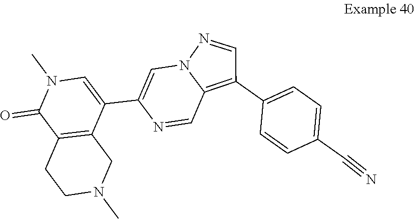 [SMILES: CN1CCc2c(c(-c3cn4ncc(-c5ccc(C#N)cc5)c4cn3)cn(C)c2=O)C1]

To a solution of Example 39 (190 mg, 0.383 mmol, TFA salt) in MeOH (5 mL) was added DIPEA (148 mg, 1.15 mmol) and stirred at 25° C. for 0.5 hour, then HOAc (69 mg, 1.2 mmol), and 37% aqueous formaldehyde (155 mg, 1.91 mmol) were added to the reaction mixture. The reaction mixture was stirred at 25° C. for 1.5 hours. NaBH₃CN (72 mg, 1.2 mmol) was added and the mixture was stirred at 25° C. for another 2 hours. The reaction mixture was quenched with water (20 mL) and extracted with DCM (20 mL×3). The combined organic layer was washed with brine (20 mL), dried over anhydrous Na₂SO₄, filtered and concentrated. The residue was purified by prep-HPLC (Method C; 0.225% FA as an additive) and lyophilized to give the title compound (17.7 mg, yield: 10%, FA salt) as a yellow solid.

¹H NMR (400 MHz, DMSO-d₆) δ 2.26 (3H, s), 2.54-2.57 (4H, m), 3.48-3.50 (5H, m), 7.84 (1H, s), 7.94 (2H, d, J=8.0 Hz), 8.09 (2H, d, J=8.0 Hz), 8.75 (1H, s), 8.96 (1H, d, J=1.2 Hz), 9.60 (1H, d, J=1.6 Hz).

The following compounds were synthesized analogously to Example 40

| Example | | | |
|---|---|---|---|
| No. | Structure | Name | ¹H NMR (400 MHz) |
| 43 | 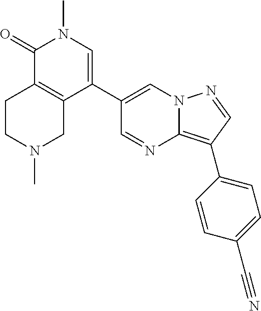 [SMILES: CN1CCc2c(c(-c3cnc4c(-c5ccc(C#N)cc5)cnn4c3)cn(C)c2=O)C1] | 4-(6-(2,6-dimethyl-1- oxo-1,2,5,6,7,8- hexahydro-2,6- naphthyridin-4- yl)pyrazolo[1,5- a]pyrimidin-3- yl)benzonitrile | DMSO-d₆) δ 2.27 (3H, s), 2.54- 2.57 (2H, m), 2.57-2.62 (2H, m), 3.29 (2H, s), 3.49 (3H, s), 7.76 (1H, s), 7.92 (2H, d, J = 8.8 Hz), 8.42 (2H, d, J = 8.4 Hz), 8.78 (1H, d, J = 2.4 Hz), 8.99 (1H, s), 9.28 (1H, d, J = 2.4 Hz). |
| 45 | 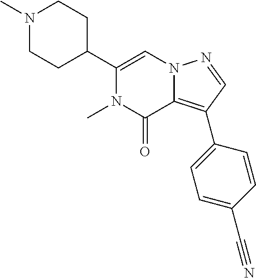 [SMILES: CN1CCC(c2cn3ncc(-c4ccc(C#N)cc4)c3c(=O)n2C)CC1] | 4-(5-methyl-6-(1- methylpiperidin-4- yl)-4-oxo-4,5- dihydropyrazolo[1,5- a]pyrazin-3- yl)benzonitrile | DMSO-d₆; δ 1.69-1.81 (2H, m), 2.00-2.09 (2H, m), 2.55 (3H, s), 2.89-2.97 (1H, m), 3.19-3.26 (4H, m), 3.51 (3H, s), 7.67 (1H, s), 7.87 (2H, d, J = 8.8 Hz), 8.09 (2H, d, J = 8.8 Hz), 8.34 (1H, s). |
| 59 | 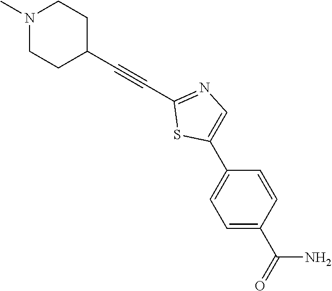 [SMILES: CN1CCC(C#Cc2ncc(-c3ccc(C(N)=O)cc3)s2)CC1] | 4-(2-((1- methylpiperidin-4- yl)ethynyl)thiazol-5- yl)benzamide | DMSO-d₆; δ 1.62-1.73 (2H, m), 1.86-1.95 (2H, m), 2.06-2.17 (2H, m), 2.18 (3H, s), 2.55-2.66 (2H, m), 2.75-2.80 (1H, m), 7.45 (1 H, brs), 7.78 (2H, d, J = 8.4 Hz), 7.94 (2H, d, J = 8.4 Hz), 8.06 (1H, brs), 8.38 (1H, s). |

### Example 41

### tert-butyl 8-(3-(4-cyanophenyl)pyrazolo[1,5-a]pyrimnidin-6-yl)-6-methyl-5-oxo-3,4,5,6-tetrahydro-2,6-naphthyridine-2(1H)-carboxylate

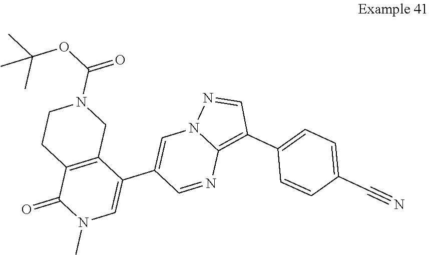 [SMILES: Cn1cc(-c2cnc3c(-c4ccc(C#N)cc4)cnn3c2)c2c(c1=O)CCN(C(=O)OC(C)(C)C)C2]

### Step 1. Synthesis of 6-bromo-3-iodopyrazolo[1,5-a]pyrimidine

To a solution of 6-bromnopyrazolo[1,5-a]pyrimidine (400 mg, 2.02 mmol) in DMF (5 mL) was added NIS (500 mg, 2.22 mmol). The mixture was stirred at 25° C. for 12 hours. The reaction mixture was poured into water (50 mL) and filtered. The solid was washed with water (10 mL×2) and dried to give 6-bromno-3-iodopyrazolo[1,5-a]pyrimidine (500 mg, yield: 760%) as a yellow solid.

¹H NMR (400 MHz, DMSO-d₆) δ 8.34 (1H, s), 8.68 (1H, d, J=2.0 Hz), 9.65 (1H, d, J=2.0 Hz).

### Step 2. Synthesis of 4-(6-bromopyrazolo[1,5-a]pyrimidin-3-yl)benzonitrile

A mixture of 6-bromo-3-iodopyrazolo[1,5-a]pyrimidine (500 mg, 1.54 mmol), (4-cyanophenyl)boronic acid (272 mg, 1.85 mmol), Pd(dppf)Cl₂ (113 mg, 0.154 mmol) and Na₂CO₃ (327 mg, 3.09 mmol) in dioxane (8 mL) and H₂O (1 mL) was degassed and purged with N₂ for 3 times, and then the mixture was stirred at 90° C. for 5 hours under N₂ atmosphere. The reaction mixture was concentrated and the residue was diluted with H₂O (30 mL) and extracted with EtOAc (50 mL×3). The combined organic layers were washed with brine (60 mL), dried over anhydrous Na₂SO₄, filtered and concentrated. The residue was purified by silica gel column (0% to 60% EtOAc in PE) to give 4-(6-bromopyrazolo[1,5-a]pyrimidin-3-yl)benzonitrile (170 mg, yield: 37%) as a yellow solid.

¹H NMR (400 MHz, DMSO-d₆) δ 7.92 (2H, d, J=8.8 Hz), 8.35 (2H, d, J=8.8 Hz), 8.83 (1H, d, J=2.0 Hz), 8.95 (1H, s), 9.73 (1H, d, J=2.0 Hz).

### Step 3. Synthesis of 4-(6-(4,4,5,5-tetramethyl-1,3,2-dioxaborolan-2-yl)pyrazolo[1,5-a]pyrimidin-3-yl)benzonitrile

A mixture of 4-(6-bromopyrazolo[1,5-a]pyrimidin-3-yl)benzonitrile (120 mg, 0.401 mmol), Bis-Pin (122 mg, 0.481 mmol), Pd₂(dba)₃ (37 mg, 0.040 mmol), PCy₃ (23 mg, 0.080 mmol) and KOAc (79 mg, 0.80 mmol) in 1, 4-dioxane (4 mL) was degassed and purged with N₂ for 3 times, and then the mixture was stirred at 100° C. for 2 hours under N₂ atmosphere. The reaction mixture was suspended in 1, 4-dioxane and filtered. The filtrate was concentrated to give 4-(6-(4,4,5,5-tetramethyl-1,3,2-dioxaborolan-2-yl)pyrazolo[1,5-a]pyrimidin-3-yl)benzonitrile (140 mg, crude) as a yellow solid.

### Step 4. Synthesis of tert-butyl 8-(3-(4-cyanophenyl)pyrazolo[1,5-a]pyrimidin-6-yl)-6-methyl-5-oxo-3,4,5,6-tetrahydro-2,6-naphthyridine-2(1H)-carboxylate

A mixture of 4-(6-(4,4,5,5-tetramethyl-1,3,2-dioxaborolan-2-yl)pyrazolo[1,5-a]pyrimidin-3-yl)benzonitrile (124 mg, 0.361 mmol), tert-butyl 8-bromo-6-methyl-5-oxo-3,4,5,6-tetrahydro-2,6-naphthyridine-2(1H)-carboxylate (140 mg, 0.400 mmol), XPhos-Pd-G₃ (34 mg, 0.040 mmol) and K₂CO₃ (111 mg, 0.802 mmol) in 1, 4-dioxane (4 mL) and H₂O (0.5 mL) was degassed and purged with N₂ for 3 times, and then the mixture was stirred at 100° C. for 14 hours under N₂ atmosphere. The reaction mixture was suspended in CH₃OH (50 mL) and filtered. The filtrate was concentrated and the residue was purified by silica gel column (0% to 100% EtOAc in PE) to give the title compound (80 mg, yield: 41%) as a yellow solid.

### Example 47

### 4-(6-(1-methylpiperidin-4-yl)-4-oxo-4,5-dihydropyrazolo[1,5-a]pyrazin-3-yl)benzonitrile

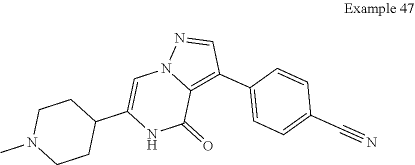 [SMILES: CN1CCC(c2cn3ncc(-c4ccc(C#N)cc4)c3c(=O)[nH]2)CC1]

### Step 1. Synthesis of tert-butyl 4-(3-(4-cyanophenyl)-4-oxo-4,5-dihydropyrazolo[1,5-a]pyrazin-6-yl)-3,6-dihydropyridine-1(2H)-carboxylate

A mixture of 4-(6-chloro-4-oxo-4,5-dihydropyrazolo[1,5-a]pyrazin-3-yl)benzonitrile (see Example 16; 800 mg, 2.96 mmol), tert-butyl 4-(4,4,5,5-tetramethyl-1,3,2-dioxaborolan-2-yl)-3,6-dihydropyridine-1(2H)-carboxylate (1.37 g, 4.43 mmol), Xphos-Pd-G₃ (250 mg, 0.296 mmol) and K₂CO₃ (817 mg, 5.91 mmol) in 1,4-dioxane (8 mL) and H₂O (1 mL) was degassed and purged with N₂ for 3 times, then the mixture was stirred at 90° C. for 12 hours under N₂ atmosphere. The reaction mixture was diluted with H₂O (50 mL) and extracted with EtOAc (100 mL×3). The combined organic layers were dried over anhydrous Na₂SO₄, filtered and concentrated. The residue was purified by Combi Flash (0% to 100% EtOAc in PE) to give tert-butyl 4-(3-(4-cyanophenyl)-4-oxo-4,5-dihydropyrazolo[1,5-a]pyrazin-6-yl)-3,6-dihydropyridine-1(2H)-carboxylate (670 mg, yield: 54%) as a yellow solid.

### Step 2. Synthesis of tert-butyl 4-(3-(4-cyanophenyl)-4-oxo-4,5-dihydropyrazolo[1,5-a]pyrazin-6-yl)piperidine-1-carboxylate

To a solution of tert-butyl 4-(3-(4-cyanophenyl)-4-oxo-4,5-dihydropyrazolo[1,5-a]pyrazin-6-yl)-3,6-dihydropyridine-1(2H)-carboxylate (200 mg, 0.479 mmol) in THF (20 mL) was added 10% Pd/C (100 mg) and degassed and purged with H₂ for 3 times. The mixture was hydrogenated (15 psi) at 25° C. for 16 hours. The mixture was filtered through a pad of celite and the filtrate was concentrated. The residue was purified by Combi Flash (0% to 60% EtOAc in PE) to give tert-butyl 4-(3-(4-cyanophenyl)-4-oxo-4,5-dihydropyrazolo[1,5-a]pyrazin-6-yl)piperidine-1-carboxylate (100 mg, yield: 50%) as a white solid.

### Step 3. Synthesis of 4-(4-oxo-6-(piperidin-4-yl)-4,5-dihydropyrazolo[1,5-a]pyrazin-3-yl)benzonitrile

A mixture of tert-butyl 4-(3-(4-cyanophenyl)-4-oxo-4,5-dihydropyrazolo[1,5-a]pyrazin-6-yl)piperidine-1-carboxylate (100 mg, 0.238 mmol) in TFA (1 mL) and DCM (4 mL) was stirred at 25° C. for 1.5 hours. The reaction mixture was concentrated to give 4-(4-oxo-6-(piperidin-4-yl)-4,5-dihydropyrazolo[1,5-a]pyrazin-3-yl)benzonitrile (100 mg, crude, TFA salt) as a yellow solid.

### Step 4. Synthesis of 4-(6-(I-methylpiperidin-4-yl)-4-oxo-4,5-dihydropyrazolo[1,5-a]pyrazin-3-yl)benzonitrile

To a solution of 4-(4-oxo-6-(piperidin-4-yl)-4,5-dihydropyrazolo[1,5-a]pyrazin-3-yl)benzonitrile (100 mg, 0.313 mmol) in MeOH (5 mL) was added HOAc (19 mg, 0.31 mmol) and 37% aqueous formaldehyde (127 mg, 1.57 mmol) and stirred at 25° C. for 0.5 hour. NaBH₃CN (60 mg, 0.94 mmol) was added to the reaction mixture and stirred at 25° C. for another 1 hour. The reaction mixture was concentrated. The residue was purified by prep-HPLC (Method C; 0.225% FA as an additive), then lyophilized to give the title compound (35.19 mg, yield: 34%, FA salt) as an off-white solid.

¹H NMR (400 MHz, DMSO-d₆) δ 1.63-1.76 (2H, m), 1.86-1.94 (2H, m), 1.97-2.09 (2H, m), 2.25 (3H, s), 2.45-2.50 (1H, m), 2.90-2.95 (2H, m), 7.55 (1H, s), 7.87 (2H, d, J=8.4 Hz), 8.11-8.18 (3H, m), 8.33 (1H, s), 11.44 (1H, brs).

### Example 48

### ethyl 4-(7-benzoyl-7,8,9,10-tetrahydropyrazolo[5,1-f][1,6]naphthyridin-1-yl)benzoate

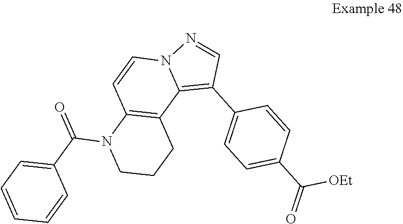 [SMILES: CCOC(=O)c1ccc(-c2cnn3ccc4c(c23)CCCN4C(=O)c2ccccc2)cc1]

To a solution of Int-14 (490 mg, 1.38 mmol) and (4-(ethoxycarbonyl)phenyl)boronic acid (534 mg, 2.75 mmol) in 1, 4-dioxane (10 mL) and H₂O (2 mL) was added Na₂CO₃ (364 mg, 3.44 mmol) and Xphos-Pd-G₃ (175 mg, 0.206 mmol) under N₂ atmosphere, the mixture was stirred at 90° C. for 16 hours under N₂ atmosphere. The reaction mixture was concentrated. The residue was purified by flash silica gel chromatography (ISCO®; 12 g SepaFlash® Silica Flash Column, Eluent of ˜30% Ethyl acetate/Petroleum ether gradient @40 mL/min) to give the title compound (350 mg, yield: 46%) as a yellow solid.

### Example 49

### 4-(7-benzyl-7,8,9,10-tetrahydropyrazolo[5,1-f][1,6]naphthyridin-1-yl)benzoic acid

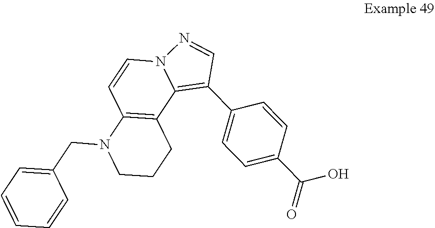 [SMILES: O=C(O)c1ccc(-c2cnn3ccc4c(c23)CCCN4Cc2ccccc2)cc1]

To a solution of Example 48 (300 mg, 0.705 mmol) in MeOH (4 mL), THF (4 mL) and H₂O (4 mL) was added NaOH (226 mg, 5.64 mmol), the mixture was stirred at 45° C. for 2 hours. After cooled to room temperature, BnBr (724 mg, 4.23 mmol) was added to the reaction mixture and the mixture was stirred at 20° C. for 16 hours. The reaction mixture was acidified with 1N aqueous HCl to pH=4 and concentrated. The residue was purified by prep-HPLC (Method D; 0.05% HCl as an additive) and lyophilized to give the title compound (27 mg, yield: 10%) as a yellow solid.

¹H NMR (400 MHz, DMSO-d₆) δ 1.71-1.88 (2H, m), 3.34-3.42 (4H, m), 4.64 (2H, s), 6.56 (1H, d, J=7.6 Hz), 7.22-7.29 (3H, m), 7.31-7.39 (2H, m), 7.47 (2H, d, J=8.4 Hz), 7.81 (1H, s), 7.93 (2H, d, J=8.4 Hz), 8.30 (1H, d, J=7.6 Hz), 12.82 (1H, brs).

### Example 50 ethyl 4-(7-(3,6-dihydro-2H-pyran-4-yl)imidazo[1,2-b]pyridazin-3-yl)benzoate

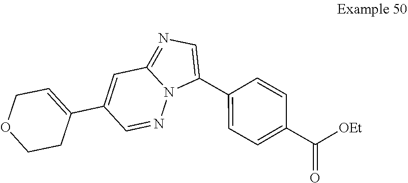 [SMILES: CCOC(=O)c1ccc(-c2cnc3cc(C4=CCOCC4)cnn23)cc1]

### Step 1. Synthesis of ethyl 4-(7-chloroimidazo[1,2-b]pyridazin-3-yl)benzoate

A mixture of Int-10 (500 mg, 1.79 mmol), (4-(ethoxycarbonyl)phenyl)boronic acid (382 mg, 1.97 mmol), Pd(dppf)Cl₂ (131 mg, 0.180 mmol) and Na₂CO₃ (379 mg, 3.58 mmol) in 1, 4-dioxane (8 mL) and H₂O (1 mL) was degassed and purged with N₂ for 3 times, and then the mixture was stirred at 90° C. for 12 hours under N₂ atmosphere. The reaction mixture was concentrated and the residue was purified by silica gel column (0% to 20% EtOAc in PE) to give ethyl 4-(7-chloroimidazo[1,2-b]pyridazin-3-yl)benzoate (250 mg, yield: 46%) as a yellow solid.

¹H NMR (400 MHz, DMSO-d₆) δ 1.35 (3H, t, J=7.2 Hz), 4.35 (2H, q, J=7.2 Hz), 8.10 (2H, d, J=8.4 Hz), 8.32 (2H, d, J=8.4 Hz), 8.47 (1H, s), 8.58 (1H, d, J=2.4 Hz), 8.84 (1H, d, J=2.4 Hz).

### Step 2. Synthesis of ethyl 4-(7-(3,6-dihydro-2H-pyran-4-yl)imidazo[1,2-b]pyridazin-3-yl)benzoate

A mixture of ethyl 4-(7-chloroimidazo[1,2-b]pyridazin-3-yl)benzoate (150 mg, 0.497 mmol), 2-(3,6-dihydro-2H-pyran-4-yl)-4,4,5,5-tetramethyl-1,3,2-dioxaborolane (157 mg, 0.746 mmol), XPhos-Pd-G₃ (42 mg, 0.050 mmol) and K₂CO₃ (137 mg, 0.994 mmol) in 1,4-dioxane (4 mL) and H₂O (0.5 mL) was degassed and purged with N₂ for 3 times, and then the mixture was stirred at 90° C. for 16 hours under N₂ atmosphere. The reaction mixture was concentrated and the residue was purified by silica gel column (0% to 80% EtOAc in PE) to give the title compound (100 mg, yield: 57%) as a yellow solid.

### Example 52

### ethyl 4-(7-(tetrahydro-2H-pyran-4-yl)imidazo[1,2-b]pyridazin-3-yl)benzoate

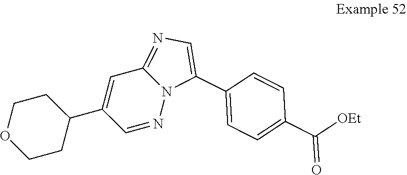 [SMILES: CCOC(=O)c1ccc(-c2cnc3cc(C4CCOCC4)cnn23)cc1]

To a solution of Example 50 (100 mg, 0.290 mmol) in THF (5 mL) was added Pd/C (50 mg, 10% purity). Then the mixture was degassed and purged with H₂ for 3 times and stirred at 25° C. for 5 hours under H₂ (15 Psi) atmosphere. The reaction mixture was filtered and the solid was washed with THF (5 mL×3), the filtrate was concentrated to give the title compound (80 mg, yield: 79%) as a yellow solid.

### Example 54

### 4-(2-((tetrahydro-2H-pyran-4-yl)ethynyl)thiazol-5-yl)benzoic acid

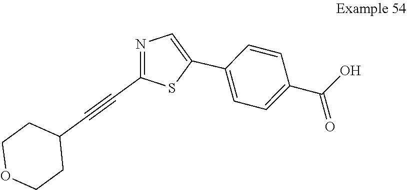 [SMILES: O=C(O)c1ccc(-c2cnc(C#CC3CCOCC3)s2)cc1]

### Step 1. Synthesis of ethyl 4-(2-((tetrahydro-2H-pyran-4-yl)ethynyl)thiazol-5-yl)benzoate

A mixture of Int-11 (400 mg, 1.28 mmol), Pd(PPh₃)₂Cl₂ (90 mg, 0.13 mmol), CuI (49 mg, 0.26 mmol), Et₃N (648 mg, 6.41 mmol) in THF (15 mL) was degassed and purged with N₂ for 3 times. Then 4-ethynyltetrahydro-2H-pyran (155 mg, 1.41 mmol) was added dropwise and the resulting mixture was stirred at 25° C. for 16 hours under N₂ atmosphere. The reaction mixture was concentrated, then diluted with water (60 mL) and extracted with EtOAc (60 mL×3). The combined organic layers were dried over anhydrous Na₂SO₄, filtered and concentrated. Then the residue was purified by silica gel column (PE/EtOAc=3/1) to afford ethyl 4-(2-((tetrahydro-2H-pyran-4-yl)ethynyl)thiazol-5-yl)benzoate (310 mg, yield: 71%) as a yellow solid.

¹H NMR (400 MHz, DMSO-d₆) δ 1.33 (3H, t, J=7.2 Hz), 1.56-1.73 (2H, m), 1.84-1.93 (2H, m), 2.88-3.14 (1H, m), 3.37-3.58 (2H, m), 3.73-3.89 (2H, m), 4.33 (2H, q, J=7.2 Hz), 7.85 (2H, d, J=8.4 Hz), 8.01 (2H, d, J=8.0 Hz), 8.43 (1H, s).

### Step 2. Synthesis of 4-(2-((tetrahydro-2H-pyran-4-yl)ethynyl)thiazol-5-yl)benzoic acid

A solution of ethyl 4-(2-((tetrahydro-2H-pyran-4-yl)ethynyl)thiazol-5-yl)benzoate (310 mg, 0.908 mmol) and LiOH·H₂O (114 mg, 2.72 mmol) in H₂O (2 mL), MeOH (2 mL) and THF (2 mL) was stirred at 25° C. for 2 hours. The reaction mixture was acidified with 1N aqueous HCl to pH=3 and concentrated. Then the residue was diluted with water (30 mL) and extracted with EtOAc (30 mL×3). The combined organic layers were dried over anhydrous Na₂SO₄, filtered and concentrated. The residue was purified by prep-HPLC (0.05% HCl as an additive) and triturated with CH₃CN (2 mL), then lyophilized to afford the title compound (34 mg, yield: 26%) as a yellow solid.

¹H NMR (400 MHz, DMSO-d₆) δ 1.60-1.72 (2H, m), 1.84-1.93 (2H, m), 3.00-3.09 (1H, m), 3.42-3.50 (2H, m), 3.75-3.87 (2H, m), 7.82 (2H, d, J=8.4 Hz), 7.99 (2H, d, J=8.8 Hz), 8.42 (1H, s), 13.12 (1H, brs).

### Example 56

### 4-(2-(tetrahydro-2H-pyran-4-carboxamido)thiazol-5-yl)benzoic acid

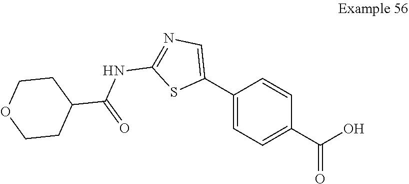 [SMILES: O=C(O)c1ccc(-c2cnc(NC(=O)C3CCOCC3)s2)cc1]

### Step 1. Synthesis of ethyl 4-(2-(tetrahydro-2H-pyran-4-carboxamido)thiazol-5-yl)benzoate

To a solution of ethyl 4-(2-aminothiazol-5-yl)benzoate (200 mg, 0.805 mmol) in DCM (5 mL) was added TEA (0.3 mL) and tetrahydropyran-4-carbonyl chloride (144 mg, 0.966 mmol) and then the resulting mixture was stirred at 20° C. for 2 hours. The reaction mixture was concentrated and the residue was purified by silica gel column (PE/EtOAc=0/1) to afford ethyl 4-(2-(tetrahydro-2H-pyran-4-carboxamido)thiazol-5-yl)benzoate (330 mg, yield: 79%) as a yellow solid.

### Step 2. Synthesis of 4-(2-(tetrahydro-2H-pyran-4-carboxamido)thiazol-5-yl)benzoic acid

To a solution of ethyl 4-(2-(tetrahydro-2H-pyran-4-carboxamido)thiazol-5-yl)benzoate (100 mg, 0.277 mmol) in THF (2 mL), MeOH (2 mL) and H₂O (1 mL) was added LiOH·H₂O (23 mg, 0.56 mmol) and then stirred at 20° C. for 2 hours. To the reaction mixture was added H₂O (2 mL) and concentrated, then acidified with 1N aqueous HCl to pH=5 and filtered. The solid was lyophilized to afford the title compound (7.4 mg, yield: 8%) as a white solid.

¹H NMR (400 MHz, DMSO-d₆) δ 1.56-1.84 (4H, m), 2.69-2.82 (1H, m), 3.37 (2H, m), 3.86-3.94 (2H, m), 7.46 (2H, d, J=7.6 Hz), 7.79-7.88 (3H, m), 12.15 (1H, brs).

### Example 60

### N-[5-4-cyano-3-isopropoxy-phen)thiazol-2-yl]-1-methyl-6-oxo-pyridine-3-carboxamide

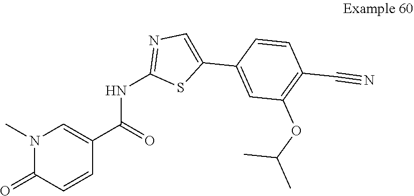 [SMILES: CC(C)Oc1cc(-c2cnc(NC(=O)c3ccc(=O)n(C)c3)s2)ccc1C#N]

### Step 1: Synthesis of 4-[(E)-2-ethoxyethenyl]-2-propan-2-yloxybenzonitrile

A mixture of 4-bromo-2-isopropoxy-benzonitrile (2.0 g, 8.33 mmol), (E)-1-ethoxyethen-2-boronic acid pinacol ester (2.47 g, 12.4 mmol), dichloro[1,1′-bis(diphenylphosphino)ferrocene]palladium(II) dichloromethane adduct (340 mg, 0.42 mmol) and sodium carbonate (0.89 mL, 20.8 mmol) in 1,4-Dioxane (25 mL) and water (5 mL) was stirred at 90° C. for 2 hr. The mixture was diluted with EtOAc, washed with water and brine, dried over Na₂SO₄ then concentrated. The residue was purified by flash chromatography (120 g, eluting with 0-20% EtOAc in hexane over 30 min) to give 4-[(E)-2-ethoxyethenyl]-2-propan-2-yloxybenzonitrile (1.61 g, 83% yield) as a clear viscous oil. LCMS: RT=0.84 min; ES-MS [M+1]⁺: 232.0

### Step 2: Synthesis of 4-(2-amino-1,3-thiazol-5-yl)-2-propan-2-yloxybenzonitrile

N-Bromosuccinimide (1.35 g, 7.61 mmol) was added to 4-[(E)-2-ethoxyvinyl]-2-isopropoxy-benzonitrile (1.6 g, 6.92 mmol) in 1,4-Dioxane (20 mL) and Water (5 mL) at 0° C. then stirred at 0° C. for 20 min. Thiourea (579 mg, 7.61 mmol) was added and the solution stirred at 80° C. for 1 hr. The solution was then diluted with EtOAc, washed with H₂O and brine, dried over Na₂SO₄ then concentrated to give 4-(2-amino-1,3-thiazol-5-yl)-2-propan-2-yloxybenzonitrile (1.55 g, 86% yield) as a tan solid. LCMS: RT=0.54 min, ES-MS [M+1]⁺: 260.1; ¹H NMR (400 MHz, DMSO-d₆) δ 9.26-8.98 (bs, 1H), 8.04 (s, 1H), 7.72 (d, J=8.1 Hz, 1H), 7.41 (s, 1H), 7.14 (d, J=8.1 Hz, 1H), 4.97 (sept, J=5.8 Hz, 1H), 1.33 (d, J=5.6 Hz, 6H).

### Step 3: Synthesis of N-[5-(4-cyano-3-isopropoxy-phenyl)thiazol-2-yl]-1-methyl-6-oxo-pyridine-3-carboxamide

A mixture of 1-methyl-6-oxo-pyridine-3-carboxylic acid (9 mg, 0.06 mmol), 4-(2-aminothiazol-5-yl)-2-isopropoxy-benzonitrile (15. mg, 0.06 mmol), 1-(3-dimethylpropyl)-3-ethylcarbodiimide hydrochloride (16 mg, 0.09 mmol) and 1-hydroxybenzotriazole hydrate (13.3 mg, 0.09 mmol) in DMF (0.5 mL) was stirred at 70° C. for 2 hr. The solution was purified by prep HPLC (5-80% MeCN in 0.05% NH₄OH (aq) over 10 min) to give N-[5-(4-cyano-3-isopropoxy-phenyl)thiazol-2-yl]-1-methyl-6-oxo-pyridine-3-carboxamide as a clear glass. LCMS: RT=0.75 min; ES-MS [M+1]⁺: 445.0. NMR (400 MHz, DMSO-d₆) δ 8.18-8.05 (m, 3H), 8.02 (d, J=6.3 Hz, 1H), 7.82 (s, 1H), 7.77-7.72 (m, 1H), 7.64 (d, J=8.5 Hz, 1H), 7.54 (s, 1H), 3.35-3.28 (m, 1H), 2.39 (s, 3H), 1.33 (d, J=5.9 Hz, 6H).

### Example 61

### N-(5-(4-cyano-3-isopropoxyphenyl)thiazol-2-yl)-1-methyl-2-oxo-1,2-dihydropyridine-4-carboxamide

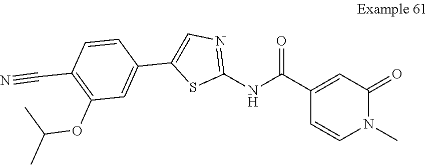 [SMILES: CC(C)Oc1cc(-c2cnc(NC(=O)c3ccn(C)c(=O)c3)s2)ccc1C#N]

A mixture of 1-methyl-2-oxopyridine-4-carboxylic acid (14.2 mg, 0.092 mmol), 4-(2-aminothiazol-5-yl)-2-isopropoxy-benzonitrile (20 mg, 0.08 mmol), 1-(3-dimethylpropyl)-3-ethylcarbodiimide hydrochloride (22.2 mg, 0.12 mmol) and 1-hydroxybenzotriazole hydrate (17.7 mg, 0.12 mmol) in DMF (0.5 mL) was stirred at 90° C. for 2 hr. The solutions was purified by semi-prep HPLC (5-60% MeCN in 0.05% NH₄OH (aq) over 10 min) to give N-[5-(7-cyano-1H-indol-4-yl)-, 3-thiazol-2-yl]-1-methyl-2-oxopyridine-4-carboxamide (3.1 mg, 10% yield) as a clear glass. LCMS: RT=0.71 min; ES-MS [M+1]⁺: 376.1.

### Example 62

### N-(5-(4-cyano-3-isopropoxyphenyl)thiazol-2-yl)-1-isopropyl-2-oxo-1,2-dihydropyridine-4-carboxamide

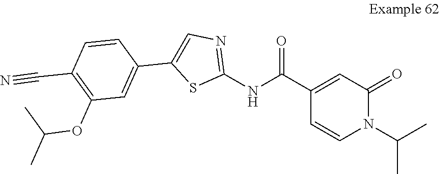 [SMILES: CC(C)Oc1cc(-c2cnc(NC(=O)c3ccn(C(C)C)c(=O)c3)s2)ccc1C#N]

A mixture of 2-oxo-1-propan-2-ylpyridine-4-carboxylic acid (10 mg, 0.06 mmol), 4-(2-aminothiazol-5-yl)-2-isopropoxy-benzonitrile (15 mg, 0.06 mmol), 1-(3-dimethylpropyl)-3-ethylcarbodiimide hydrochloride (16 mg, 0.09 mmol) and 1-hydroxybenzotriazole hydrate (13 mg, 0.09 mmol) in DMF (0.5 mL) was stirred at 90° C. for 1 hr. The solution was then purified by prep HPLC (5-70% MeCN in 0.05% NH₄OH (aq) over 10 min) to give N-[5-(4-cyano-3-propan-2-yloxyphenyl)-1,3-thiazol-2-yl]-2-oxo-1-propan-2-ylpyridine-4-carboxamide (12 mg, 52% yield) as a tan solid. LCMS: RT=0.91 min; ES-MS [M+1]⁺: 423.3.

### Example 63

### (2R,6S)-N-(5-(4-cyano-3-isopropoxyphenyl)thiazol-2-yl)-2,6-dimethylmorpholine-4-carboxamide

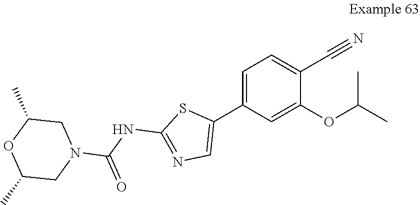 [SMILES: CC(C)Oc1cc(-c2cnc(NC(=O)N3C[C@@H](C)O[C@@H](C)C3)s2)ccc1C#N]

A mixture of 4-(2-aminothiazol-5-yl)-2-isopropoxy-benzonitrile (20 mg, 0.08 mmol), 4-nitrophenylchloroformate (19 mg, 0.09 mmol) and pyridine (0.01 mL, 0.12 mmol) in MeCN (0.5 mL) was stirred at RT for 2 hr. (2R,5S)-2,6-dimethylmorpholine (35 mg, 0.31 mol) was added and the solutions stirred at RT for 20 min. The solution was purified by prep HPLC (5-70% MeCN in 0.05% NH₄OH (aq) over 10 min) to give (2R,6S)-N-[5-(4-cyano-3-propan-2-yloxyphenyl)-1,3-thiazol-2-yl]-2,6-dimethylmorpholine-4-carboxamide (10.2 mg, 33% yield) as a white solid. LCMS: RT=0.83 min; ES-MS [M+1]⁺: 401.1, NMR (400 MHz, DMSO-d₆) δ 11.39-11.11 (bs, 1H), 8.10 (s, 1H), 7.69 (d, J=2.9 Hz, 1H), 7.40 (s, 1H), 7.20 (d, J=8.4 Hz, 1H), 4.99-4.92 (m, 1H), 4.18-4.10 (m, 2H), 3.56-3.48 (m, 2H), 2.56-2.49 (m, 2H), 1.34 (d, J=5.8 Hz, 6H), 1.11 (d, J=5.8 Hz, 6H).

### Intermediates of Formula (III)

### Intermediate 1

### [3,4′-bipyridin]-6(1H)-one

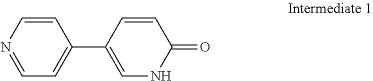 [SMILES: O=c1ccc(-c2ccncc2)c[nH]1]

### Step 1. Synthesis of 6-methoxy-3,4′-bipyridine

To a mixture of (6-methoxypyridin-3-yl)boronic acid (2.00 g, 13.3 mmol), 4-chloropyridine (3.06 g, 20.0 mmol) in DMA (30 mL) was added Cs₂CO₃ (8.69 g, 26.7 mmol), XPhos (636 mg, 1.33 mmol) and Pd₂(dba)₃ (610 mg, 0.667 mmol), then the mixture was degassed and purged with N₂ for 3 times and the mixture was stirred at 80° C. for 12 hours under N₂ atmosphere. The reaction mixture was suspended in CH₃OH (50 mL) and filtered. The filtrate was concentrated and the residue was diluted with H₂O (50 mL) and extracted with EtOAc (70 mL×3). The combined organic layers were washed with brine (100 mL), dried over anhydrous Na₂SO₄, filtered and concentrated. Then the residue was purified by silica gel column (0% to 40% EtOAc in PE) to give 6-methoxy-3,4′-bipyridine (1.80 g, yield: 72%) as a yellow solid.

¹H NMR (400 MHz, CDCl₃) δ 4.01 (3H, s), 6.88 (1H, d, J=8.4 Hz), 7.36-7.55 (2H, m), 7.85 (1H, dd, J=8.8, 2.8 Hz), 8.37-8.56 (1H, m), 8.58-8.78 (2H, m).

### Step 2. Synthesis of [3,4′-bipyridin]-6(1H)-one

To a solution of 6-methoxy-3,4′-bipyridine (1.80 g, 9.67 mmol) in EtOH (9 mL) was added HBr (26.8 g, 109 mmol, 33% in CH₃COOH). The mixture was stirred at 90° C. for 6 hours. The reaction mixture was basified with saturated aqueous NaHCO₃ to pH=7 and extracted with EtOAc (100 mL×3). The combined organic layers were washed with brine (120 mL), dried over anhydrous Na₂SO₄, filtered and concentrated to give [3,4′-bipyridin]-6(1H)-one (1.15 g, yield: 69%) as a yellow solid.

¹H NMR (400 MHz, DMSO-d₆) δ 6.47 (1H, d, J=9.6 Hz), 7.53-7.72 (2H, m), 7.93-7.99 (1H, m), 8.00 (1H, s), 8.47-8.61 (2H, m), 12.08 (1H, brs).

### Intermediate 2

### cis-4-ethynyl-2,6-dimethyltetrahydro-2H-pyran

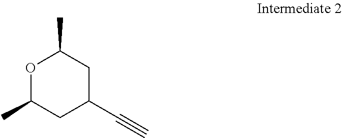

### Step 1. Synthesis of cis-2,6-dimethyltetrahydro-4H-pyran-4-one

To a solution of compound 2,6-dimethyl-4H-pyran-4-one (5.00 g, 40.3 mmol) in EtOH (50 mL) was added 10% Pd/C (500 mg) under N₂ atmosphere. The mixture was degassed and purged with H₂ for 3 times, then the mixture was hydrogenated (15 psi) at 25° C. for 16 hours. The reaction mixture was filtered and the solid was washed with EtOH (10 mL×2), the filtrate was concentrated. The residue was purified by silica gel column (PE/EtOAc=3/1) to afford cis-2,6-dimethyltetrahydro-4H-pyran-4-one (1.20 g, yield: 23%) as yellow oil.

### Step 2. Synthesis of cis-(Z)-4-(methoxymethylene)-2,6-dimethyltetrahydro-2H-pyran

To a suspension of PPh₃CH₂O CH₃Cl (2.94 g, 8.58 mmol) in THF (15 mL) was added LDA (3.22 mL, 6.44 mmol, 2M in THF) dropwise at 0° C. After the completion of the addition, the reaction mixture was stirred at 0° C. for 0.5 hour. A solution of cis-2,6-dimethyltetrahydro-4H-pyran-4-one (550 mg, 4.29 mmol) in anhydrous THF (5 mL) was added dropwise to the above reaction mixture at −65° C. The mixture was stirred at −65° C. for 1 hour, then allowed to stir at 25° C. for 17 hours. The reaction mixture was quenched with water (50 mL) and extracted with DCM (20 mL×3). The combined organic layers were washed with brine (25 mL), dried over anhydrous Na₂SO₄, filtered and concentrated. The residue was purified by silica gel column (PE/EtOAc=5/1) to afford cis-(Z)-4-(methoxymethylene)-2,6-dimethyltetrahydro-2H-pyran (500 mg, yield: 75%) as yellow oil.

¹H NMR (400 MHz, DMSO-d₆) δ 1.08-1.14 (6H, m), 1.58-1.68 (2H, m), 1.90-1.96 (1H, m), 2.52-2.56 (1H, m), 3.21-3.31 (2H, m), 3.48 (3H, s), 5.91 (1H, s).

### Step 3. Synthesis of cis-2,6-dimethyltetrahydro-2H-pyran-4-carbaldehyde

A mixture of cis-(Z)-4-(methoxymethylene)-2,6-dimethyltetrahydro-2H-pyran (300 mg, 1.92 mmol) in H₂O (0.3 mL) and HCOOH (3 mL) was stirred at 90° C. for 1 hour. The reaction mixture was cooled under an ice-bath and basified with 6N aqueous NaOH to pH=8, then extracted with DCM (10 mL×3). The combined organic layers were dried over anhydrous Na₂SO₄, filtered and concentrated. The residue was purified by silica gel column (PE/EtOAc=5/1) to afford cis-2,6-dimethyltetrahydro-2H-pyran-4-carbaldehyde (180 mg, yield: 66%) as yellow oil.

¹H NMR (400 MHz, DMSO-d₆) δ 0.91-1.01 (2H, m), 1.10-1.15 (6H, m), 1.75-1.83 (2H, m), 2.55-2.65 (1H, m), 3.43-3.47 (2H, m), 9.53 (1H, s).

### Step 4. Synthesis of cis-4-ethynyl-2,6-dimethyltetrahydro-2H-pyran

A mixture of cis-2,6-dimethyltetrahydro-2H-pyran-4-carbaldehyde (180 mg, 1.27 mmol), diethyl (1-diazo-2-oxopropyl)phosphonate (243 mg, 1.27 mmol) and K₂CO₃ (525 mg, 3.80 mmol) in MeOH (5 mL) was stirred at 25° C. for 4 hours. The reaction mixture was concentrated and the residue was purified by silica gel column (PE/EtOAc=5/1) to afford cis-4-ethynyl-2,6-dimethyltetrahydro-2H-pyran (85 mg, yield: 49%) as colorless oil.

¹H NMR (400 MHz, DMSO-d₆) δ 0.94-0.96 (2H, m), 1.02-1.08 (6H, m), 1.75-1.79 (2H, m), 2.53-2.57 (1H, m), 2.85-3.00 (1H, m), 3.38-3.43 (2H, m).

### Intermediate 3

### 5-(2-chloropyrimidin-4-yl)-1-(tetrahydro-2H-pyran-4-yl)pyridin-2(1H)-one

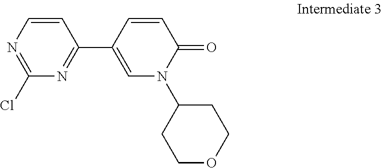 [SMILES: O=c1ccc(-c2ccnc(Cl)n2)cn1C1CCOCC1]

### Step 1. Synthesis of 4-methyl-N′-(tetrahydro-4H-pyran-4-ylidene)benzenesulfonohydrazide

To a solution of compound 4-methylbenzenesulfonohydrazide (13.0 g, 69.8 mmol) in MeOH (100 mL) was added tetrahydropyran-4-one (7.69 g, 76.8 mmol). The mixture was stirred at 25° C. for 16 hours. The reaction mixture was concentrated and the residue was triturated with PE/EtOAc (60 mL, 1/1) to give 4-methyl-N′-(tetrahydro-4H-pyran-4-ylidene)benzenesulfonohydrazide (15.5 g, yield: 83%) as a white solid.

¹H NMR (400 MHz, DMSO-d₆) δ 2.20 (2H, t, J=5.6 Hz), 2.35-2.43 (5H, m), 3.57-3.68 (4H, m), 7.40 (2H, d, J=8.0 Hz), 7.73 (2H, d, J=8.0 Hz), 10.29 (1H, brs).

### Step 2. Synthesis of 5-bromo-1-(tetrahydro-2H-pyran-4-yl)pyridin-2(1H)-one

A mixture of 5-bromo-1H-pyridin-2-one (5.00 g, 28.7 mmol), compound 4-methyl-N′-(tetrahydro-4H-pyran-4-ylidene)benzenesulfonohydrazide (15.4 g, 57.5 mmol), Cu(acac)₂ (1.50 g, 5.75 mmol) and Cs₂CO₃ (18.7 g, 57.5 mmol) in dioxane (100 mL) was degassed and purged with N₂ for 3 times, then the mixture was stirred at 110° C. for 16 hours under N₂ atmosphere. The reaction mixture was concentrated and the residue was diluted with H₂O (150 mL) and extracted with EtOAc (200 mL×3). The combined organic layers were washed with brine (200 mL), dried over anhydrous Na₂SO₄, filtered and concentrated. The residue was purified by silica gel column (0% to 53% EtOAc in PE) to give 5-bromo-1-(tetrahydro-2H-pyran-4-yl)pyridin-2(1H)-one (2.00 g, yield: 27%) as a yellow solid.

¹H NMR (400 MHz, CDCl₃) δ 1.77-1.95 (4H, m), 3.53-3.67 (2H, m), 4.07-4.19 (2H, m), 5.04-5.21 (1H, m), 6.53 (1H, d, J=10.0 Hz), 7.35 (1H, dd, J=9.6, 2.8 Hz), 7.45 (1H, d, J=2.0 Hz).

### Step 3. Synthesis of 1-(tetrahydro-2H-pyran-4-yl)-5-(4,4,5,5-tetramethyl-1,3,2-dioxaborolan-2-yl)pyridin-2(1H)-one

A mixture of 5-bromo-1-(tetrahydro-2H-pyran-4-yl)pyridin-2(1H)-one (800 mg, 3.10 mmol), Bis-Pin (1.57 g, 6.20 mmol), Pd(dppf)Cl₂ (454 mg, 0.620 mmol) and KOAc (913 mg, 9.30 mmol) in dioxane (15 mL) was degassed and purged with N₂ for 3 times, then the mixture was stirred at 90° C. for 16 hours under N₂ atmosphere. The reaction mixture was suspended in dioxane (50 mL) and filtered. The filtrate was concentrated and the residue was purified by silica gel column (0% to 35% EtOAc in PE) to give 1-(tetrahydro-2H-pyran-4-yl)-5-(4,4,5,5-tetramethyl-1,3,2-dioxaborolan-2-yl)pyridin-2(1H)-one (550 mg, yield: 58%) as a yellow gum.

### Step 4. Synthesis of 5-(2-chloropyrimidin-4-yl)-1-(tetrahydro-2H-pyran-4-yl)pyridin-2(1H)-one

A mixture of 1-(tetrahydro-2H-pyran-4-yl)-5-(4,4,5,5-tetramethyl-1,3,2-dioxaborolan-2-yl)pyridin-2(1H)-one (550 mg, 1.80 mmol), 4-bromo-2-chloro-pyrimidine (349 mg, 1.80 mmol), Pd(dppf)Cl₂ (132 mg, 0.180 mmol) and Na₂CO₃ (382 mg, 3.60 mmol) in dioxane (10 mL) and H₂O (1 mL) was degassed and purged with N₂ for 3 times, then the mixture was stirred at 90° C. for 1 hour under N₂ atmosphere. The reaction mixture was concentrated and the residue was diluted with H₂O (50 mL), then extracted with DCM (70 mL×3). The combined organic layers were washed with brine (100 mL), dried over anhydrous Na₂SO₄, filtered and concentrated. The residue was purified by silica gel column (0% to 90% EtOAc in PE) to give 5-(2-chloropyrimidin-4-yl)-1-(tetrahydro-2H-pyran-4-yl)pyridin-2(1H)-one (180 mg, yield: 31%) as a yellow solid.

### Intermediate 4

### 7-bromo-2,3-dimethylquinazolin-4(3H)-one

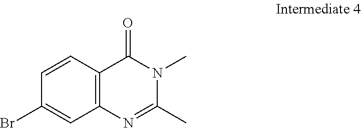

To a solution of 7-bromo-2-methylquinazolin-4(3H)-one (350 mg, 1.46 mmol) in DMF (3 mL) was added Cs₂CO₃ (720 mg, 2.2 mmol) and followed by Mel (182 μL, 2.93 mmol) at RT. After 2 h, the reaction was quenched with water and extracted with CHCl₃/IPA (3:1) 3 times. The organic was combined, dried (Na₂SO₄), filtered and concentrated to give the crude 7-bromo-2,3-dimethylquinazolin-4(3H)-one (370.2 mg, 99%). The crude was used in the next step directly. ¹H NMR (400 MHz, DMSO) δ 8.01 (d, J=8.5 Hz, 1H), 7.79 (d, J=1.9 Hz, 1H), 7.63 (dd, J=8.5, 2.0 Hz, 1H), 3.52 (s, 3H), 2.57 (s, 3H); LCMS: RT=0.58 min, ES-MS [M+H]⁺=255.1.

### Compounds of Formula (III)

### Example 1

### N-(5-(1-isobutyl-6-oxo-1,6-dihydropyridin-3-yl)thiazol-2-yl)-1-methylpiperidine-4-carboxamide

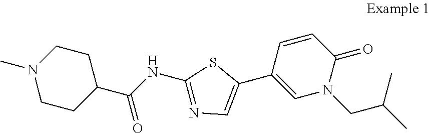 [SMILES: CC(C)Cn1cc(-c2cnc(NC(=O)C3CCN(C)CC3)s2)ccc1=O]

### Step 1. Synthesis of 1-isobutyl-5-(4,4,5,5-tetramethyl-1,3,2-dioxaborolan-2-yl)pyridin-2(1H)-one

A mixture of 5-bromo-1-isobutylpyridin-2(1H)-one (500 mg, 2.17 mmol), Bis-Pin (662 mg, 2.61 mmol), Pd(dppf)Cl₂ (159 mg, 0.217 mmol) and KOAc (640 mg, 6.52 mmol) in dioxane (10 mL) was degassed and purged with N₂ for 3 times, and then the mixture was stirred at 100° C. for 16 hours under N₂ atmosphere. The reaction mixture was filtered and the filtrate was concentrated. The residue was purified by flash silica gel chromatography (ISCO®; 20 g SepaFlash® Silica Flash Column, Eluent of 0˜40% Ethyl acetate/Petroleum ethergradient @30 mL/min) to give 1-isobutyl-5-(4,4,5,5-tetramethyl-1,3,2-dioxaborolan-2-yl)pyridin-2(1H)-one (1.20 g, yield: 72%) as yellow oil.

### Step 2. Synthesis of N-(5-(J-isobutyl-6-oxo-1,6-dihydropyridin-3-yl)thiazol-2-yl)-1-methylpiperidine-4-carboxamide

A mixture of N-(5-bromothiazol-2-yl)-1-methylpiperidine-4-carboxamide (220 mg, 0.723 mmol), 1-isobutyl-5-(4,4,5,5-tetramethyl-1,3,2-dioxaborolan-2-yl)pyridin-2(1H)-one (401 mg, 1.45 mmol), Pd(dtbpf)Cl₂ (47 mg, 0.072 mmol) and Na₂CO₃ (230 mg, 2.17 mmol) in dioxane (4 mL) and H₂O (1 mL) was degassed and purged with N₂ for 3 times, and then the mixture was stirred at 90° C. for 16 hours under N₂ atmosphere. The reaction mixture was filtered and the filtrate was concentrated. The residue was purified by prep-HPLC (Method C; 0.225% FA as an additive), then lyophilized to give the title compound (100 mg, yield: 32%, FA salt) as a yellow solid.

¹H NMR (400 MHz, CD₃OD) δ 0.98 (6H, d, J=7.2 Hz), 2.00-2.30 (5H, m), 2.75-2.85 (4H, m), 2.95-3.05 (2H, m), 3.45-3.55 (2H, m), 3.85-3.90 (2H, m), 6.63 (1H, d, J=9.6 Hz), 7.59 (1H, s), 7.80 (1H, dd, J=9.2, 2.4 Hz), 7.88 (1H, d, J=2.4 Hz), 8.50 (1H, s).

### Example 2

### 4-(2-(1-methylpiperidine-4-carboxamido)thiazol-5-yl)pyridine 1-oxide

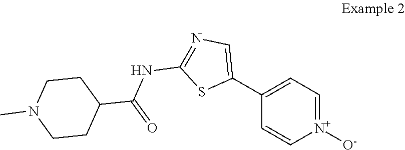 [SMILES: CN1CCC(C(=O)Nc2ncc(-c3cc[n+]([O-])cc3)s2)CC1]

A mixture of 1-methyl-N-(thiazol-2-yl)piperidine-4-carboxamide (100 mg, 0.444 mmol), 4-bromo-1-oxido-pyridin-1-ium (85 mg, 0.49 mmol), t-Bu₃PHBF₄ (26 mg, 0.089 mmol), Pd(OAc)₂ (10 mg, 0.044 mmol) and Cs₂CO₃ (289 mg, 0.888 mmol) in DMF (2 mL) was degassed and purged with N₂ for 3 times, and then the mixture was stirred at 120° C. for 2 hours under N₂ atmosphere. The reaction mixture was diluted with H₂O (10 mL) and extracted with EtOAc (15 mL×3). The combined organic layers were washed with brine (20 mL), dried over anhydrous Na₂SO₄ and concentrated. The residue was purified by prep-HPLC (0.225% FA as an additive), then lyophilized to afford the title compound (11.22 mg, yield: 8%, FA salt) as a yellow solid.

¹H NMR (400 MHz, DMSO-d₆) δ 1.59-1.71 (2H, m), 1.76-1.83 (2H, m), 1.86-1.97 (2H, m), 2.18 (3H, s), 2.43-2.48 (1H, m), 2.80-2.86 (2H, m), 7.60-7.66 (2H, m), 8.07 (1H, s), 8.12-8.24 (2H, m), 12.34 (1H, brs).

The following compound was synthesized analogously to Example 2

| Example | | | |
|---|---|---|---|
| No. | Structure | Name | ¹H NMR (400 MHz) |
| 6 | 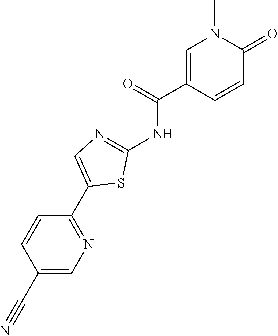 [SMILES: Cn1cc(C(=O)Nc2ncc(-c3ccc(C#N)cn3)s2)ccc1=O] | N-(5-(5- cyanopyridin-2- yl)thiazol-2-yl)-1- methyl-6-oxo-1,6- dihydropyridine-3- carboxamide | DMSO-d₆; δ 3.53 (3H, s), 6.42 (1H, d, J = 9.6 Hz), 7.19 (1H, d, J = 4.8 Hz), 7.98 (1H, dd, J = 9.2, 1.6 Hz), 8.03 (1H, d, J = 4.8 Hz), 8.49 (1H, s), 8.61-8.74 (2H, m), 9.11 (1H, s). |

### Example 3

### 3-(2-(1-methylpiperidine-4-carboxamido)thiazol-5-yl)cyclobutane-1-carboxylic acid

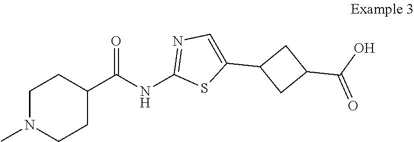 [SMILES: CN1CCC(C(=O)Nc2ncc(C3CC(C(=O)O)C3)s2)CC1]

### Step 1. Synthesis of ethyl 3-(2-((tert-butoxycarbonyl)(4-methoxybenzyl)amino)thiazol-5-yl)-3-hydroxycyclobutane-1-carboxylate

To a solution of tert-butyl (5-bromothiazol-2-yl)(4-methoxybenzyl)carbamate (500 mg, 1.25 mmol) in THF (7 mL) was added n-BuLi (0.60 mL, 1.50 mmol, 2.5M in hexane) dropwise at −78° C. under N₂ atmosphere, the mixture was stirred at −78° C. for 0.5 hour. A solution of ethyl 3-oxocyclobutane-1-carboxylate (267 mg, 1.88 mmol) in THF (3 mL) was added dropwise to the reaction mixture at −78° C., the mixture was stirred at −78° C. for another 2.5 hours under N₂ atmosphere. The reaction mixture was quenched with saturated aqueous NH₄Cl (10 mL) and diluted with H₂O (30 mL), extracted with EtOAc (40 mL×2). The combined organic layer was washed with brine (50 mL), dried over anhydrous Na₂SO₄, filtered and concentrated. The residue was purified by flash silica gel chromatography (ISCO®; 20 g SepaFlash® Silica Flash Column, Eluent of 30˜35% Ethyl acetate/Petroleum ether gradient @30 mL/min) to give 3-(2-((tert-butoxycarbonyl)(4-methoxybenzyl)amino)thiazol-5-yl)-3-hydroxycyclobutane-1-carboxylate (420 mg, yield: 70%) as light yellow gum.

### Step 2. Synthesis of ethyl 3-(2-aminothiazol-5-yl)cyclobutane-1-carboxylate

A solution of 3-(2-((tert-butoxycarbonyl)(4-methoxybenzyl)amino)thiazol-5-yl)-3-hydroxycyclobutane-1-carboxylate (420 mg, 0.908 mmol), Et₃SiH (4 mL) and TFA (4 mL) in DCM (4 mL) was stirred at 25° C. for 12 hours. The reaction mixture was concentrated and the residue was dissolved in TFA (8 mL), then stirred at 60° C. for 12 hours. The reaction mixture was concentrated and basified with aqueous 2N aqueous NaOH to pH=12 and extracted with DCM/MeOH (30 mL×3, 10/1). The combined organic layer was concentrated and the residue was purified by flash silica gel chromatography (ISCO®; 4 g SepaFlash® Silica Flash Column, Eluent of ˜5% MeOH/DCM gradient @25 mL/min) to give ethyl 3-(2-aminothiazol-5-yl)cyclobutane-1-carboxylate (125 mg, yield: 61%) as a light yellow solid.

¹H NMR (400 MHz, DMSO-d₆) δ 1.14-1.22 (3H, m), 2.04-2.17 (1H, m), 2.17-2.30 (1H, m), 2.45-2.48 (1H, m), 2.51-2.56 (1H, m), 2.99-3.16 (1H, m), 3.36-3.65 (1H, m), 3.99-4.14 (2H, m), 6.60-6.79 (3H, m).

### Step 3. Synthesis of ethyl 3-(2-(1-methylpiperidine-4-carboxamido)thiazol-5-yl)cyclobutane-1-carboxylate

To a solution of ethyl 3-(2-aminothiazol-5-yl)cyclobutane-1-carboxylate (65 mg, 0.29 mmol), 1-methylpiperidine-4-carboxylic acid (206 mg, 1.44 mmol) and Et₃N (145 mg, 1.44 mmol) in pyridine (3 mL) was added EDCI (275 mg, 1.44 mmol), the mixture was stirred at 90° C. for 12 hours. The reaction mixture was concentrated and the residue was purified by flash silica gel chromatography (ISCO®; 4 g SepaFlash® Silica Flash Column, Eluent of ˜7% MeOH/DCM gradient @25 mL/min) to give ethyl 3-(2-(1-methylpiperidine-4-carboxamido)thiazol-5-yl)cyclobutane-1-carboxylate (55 mg, yield: 55%) as a light yellow solid.

### Step 4. Synthesis of 3-(2-(1-methylpiperidine-4-carboxamido)thiazol-5-yl)cyclobutane-1-carboxylic acid

A solution of ethyl 3-(2-(1-methylpiperidine-4-carboxamido)thiazol-5-yl)cyclobutane-1-carboxylate (50 mg, 0.14 mmol) and LiOH·H₂O (12 mg, 0.28 mmol) in THF (1 mL), MeOH (1 mL) and H₂O (1 mL) was stirred at 25° C. for 12 hours. The reaction mixture was concentrated and the residue was acidified with FA to pH=4, then purified by prep-HPLC (0.225% FA as an additive) and lyophilized to give the title compound (12 mg, yield: 22%, FA salt, trans/cis=2/1) as a white solid.

¹H NMR (400 MHz, DMSO-d₆) δ 1.59-1.69 (2H, m), 1.71-1.80 (2H, m), 1.86-1.97 (2H, m), 2.16-2.34 (5H, m), 2.36-2.44 (1H, m), 2.55-2.62 (2H, m), 2.77-2.88 (2H, m), 2.99-3.14 (1H, m), 3.56-3.76 (1H, m), 7.15-7.23 (1H, m), 11.94 (1H, brs).

### Example 4

### 5-((2,3-dihydrobenzofuran-5-yl)amino)-[3,4′-bipyridin]-6(1H)-one

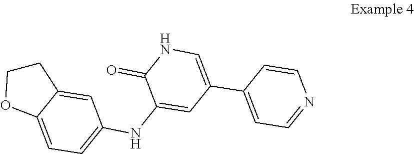 [SMILES: O=c1[nH]cc(-c2ccncc2)cc1Nc1ccc2c(c1)CCO2]

### Step 1. Synthesis of 6-methoxy-[3,4′-bipyridin]-5-amine

A mixture of 5-bromo-2-methoxypyridin-3-amine (1.50 g, 7.39 mmol), pyridin-4-ylboronic acid (1.09 g, 8.87 mmol), Pd₂(dba)₃ (338 mg, 0.369 mmol), XPhos (704 mg, 1.48 mmol) and K₃PO₄ (4.70 g, 22.2 mmol) in n-BuOH (20 mL) was degassed and purged with N₂ for 3 times. Then the reaction mixture was stirred at 110° C. under N₂ atmosphere for 3 hours. The reaction mixture was filtered and the filtrate was concentrated. The residue was purified by flash column (SiO₂, EtOAc 0% to 100% in PE) to give 6-methoxy-[3,4′-bipyridin]-5-amine (800 mg, yield: 54%) as a yellow solid.

¹H NMR (400 MHz, CDCl₃) δ 3.96 (2H, brs), 4.07 (3H, s), 7.14 (1H, d, J=2.0 Hz), 7.40-7.50 (2H, m), 7.89 (1H, d, J=2.0 Hz), 8.59-8.70 (2H, m).

### Step 2. Synthesis of N-(2,3-dihydrobenzofuran-5-yl)-6-methoxy-[3,4′-bipyridin]-5-amine

A mixture of 6-methoxy-[3,4′-bipyridin]-5-amine (500 mg, 2.48 mmol), 5-bromo-2,3-dihydrobenzofuran (495 mg, 2.48 mmol), Pd₂(dba)₃ (228 mg, 0.248 mmol), BINAP (309 mg, 0.497 mmol) and Cs₂CO₃ (1.62 g, 4.97 mmol) in anhydrous toluene (10 mL) was degassed and purged with N₂ for 3 times. Then the reaction mixture was stirred at 110° C. under N₂ atmosphere for 12 hours. The reaction mixture was filtered and the filtrate was concentrated. The crude product was purified by flash column (SiO₂, EtOAc 0% to 60% in PE) to give N-(2,3-dihydrobenzofuran-5-yl)-6-methoxy-[3,4′-bipyridin]-5-amine (450 mg, yield: 57%) as a yellow solid.

### Step 3. Synthesis of 5-((2,3-dihydrobenzofuran-5-yl)amino)-[3,4′-bipyridin]-6(1H)-one

To a mixture of N-(2,3-dihydrobenzofuran-5-yl)-6-methoxy-[3,4′-bipyridin]-5-amine (350 mg, 1.10 mmol) in EtOH (3 mL) was added 33% HBr/AcOH (3 mL) at 25° C. for one portion. The reaction mixture was stirred at 50° C. for 6 hours. The reaction mixture was basified with saturated aqueous NaHCO₃ to pH=8 and extracted with EtOAc (15 mL×5). The combined organic layer was concentrated and the residue was purified by prep-HPLC (0.04% NH₃H₂O+10 mM NH₄HCO₃ as an additive) and lyophilized to give the title compound (8 mg, yield: 2%) as an off-white solid.

¹H NMR (400 MHz, DMSO-d₆) δ 3.19 (2H, t, J=8.4 Hz), 4.52 (2H, t, J=8.4 Hz), 6.74 (1H, d, J=8.4 Hz), 7.02-7.11 (2H, m), 7.21 (1H, s), 7.34 (1H, d, J=2.0 Hz), 7.46-7.55 (3H, m), 8.45-8.57 (2H, m), 12.03 (1H, brs).

### Example 5

### 1-(3-(2-(dimethylamino)ethyl)benzyl)-[3,4′-bipyridin]-6(1H)-one

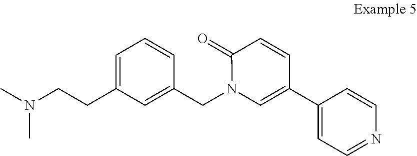 [SMILES: CN(C)CCc1cccc(Cn2cc(-c3ccncc3)ccc2=O)c1]

### Step 1. Synthesis of (3-(2-aminoethyl)phenyl)methanol

To a suspension of LiAlH₄ (1.43 g, 37.7 mmol) in THF (30 mL) was added AlCl₃ (5.02 g, 37.7 mmol) at 0° C., then a solution of methyl 3-(cyanomethyl)benzoate (3.00 g, 17.1 mmol) in Et₂O (40 mL) was added to the mixture at 0° C., and then the mixture was stirred at 25° C. for 12 hours. The reaction mixture was quenched with H₂O (1.5 mL) and 1N aqueous NaOH (1.5 mL), then suspended in CH₃OH (20 mL) and filtered. The filtrate was concentrated to give (3-(2-aminoethyl)phenyl)methanol (4.00 g, crude) as yellow gum.

### Step 2. Synthesis of (3-(2-(dimethylamino)ethyl)phenyl)methanol

To a solution of (3-(2-aminoethyl)phenyl)methanol (1.00 g, 6.61 mmol) in HCOOH (3.18 g, 66.1 mmol) was added 37% aqueous HCHO (5.37 g, 66.1 mmol) slowly at 0° C., then the mixture was stirred at 80° C. for 12 hours. The reaction mixture was basified with 50% aqueous NaOH to pH=11 and extracted with EtOAc (50 mL×3). The combined organic layer was washed with brine (80 mL), dried over anhydrous Na₂SO₄, filtered and concentrated to give (3-(2-(dimethylamino)ethyl)phenyl)methanol (200 mg, yield: 17%) as yellow gum.

¹H NMR (400 MHz, DMSO-d₆) δ 2.18 (6H, s), 2.38-2.48 (2H, m), 2.63-2.75 (2H, m), 4.40-4.53 (2H, m), 5.10-5.19 (1H, m), 7.08 (1H, d, J=7.2 Hz), 7.12 (1H, d, J=7.6 Hz), 7.16 (1H, s), 7.19-7.25 (1H, m).

### Step 3. Synthesis of 2-(3-(chloromethyl)phenyl)-N,N-dimethylethan-1-amine

To a solution of give (3-(2-(dimethylamino)ethyl)phenyl)methanol (135 mg, 0.753 mmol) in toluene (2 mL) was added SOCl₂ (246 mg, 2.07 mmol) at 0° C. The mixture was stirred at 25° C. for 12 hours. The reaction mixture was concentrated to give 2-(3-(chloromethyl)phenyl)-N,N-dimethylethan-1-amine (130 mg, yield: 74%) as a yellow solid.

### Step 4. Synthesis of 1-(3-(2-(dimethylamino)ethyl)benzyl)-[3,4′-bipyridin]-6(1H)-one

To a solution of 2-(3-(chloromethyl)phenyl)-N,N-dimethylethan-1-amine (64 mg, 0.37 mmol) in DMF (2 mL) was added K₂CO₃ (205 mg, 1.48 mmol) at 25° C. The mixture was stirred at 25° C. for 0.5 hour. Then Int-1 (130 mg, 0.555 mmol) was added to the mixture and stirred at 80° C. for 11.5 hours. The mixture was poured into water (20 mL) and extracted with extracted with EtOAc (20 mL×3). The combined organic layers were washed with brine (30 mL), dried over anhydrous Na₂SO₄, filtered and concentrated. The residue was purified by prep-HPLC (0.05% NH₃·H₂O as an additive) and lyophilized, then further purified by prep-TLC (SiO₂, DCM/MeOH=5/1) to afford the title compound (2.41 mg, yield: 2%) as yellow gum.

¹H NMR (400 MHz, CD₃OD-d₄) δ 2.21 (6H, s), 2.43-2.53 (2H, m), 2.63-2.74 (2H, m), 5.17 (2H, s), 6.61 (1H, d, J=9.6 Hz), 7.06-7.14 (2H, m), 7.15-7.23 (2H, m), 7.44-7.65 (2H, m), 7.90 (1H, dd, J=9.6, 2.8 Hz), 8.23 (1H, d, J=2.4 Hz), 8.37-8.52 (2H, m).

### Example 7

### 5-(2-((1-isobutylpiperidin-4-yl)ethynyl)pyrimidin-4-yl)-1-(tetrahydro-2H-pyran-4-yl)pyridin-2(1H)-one

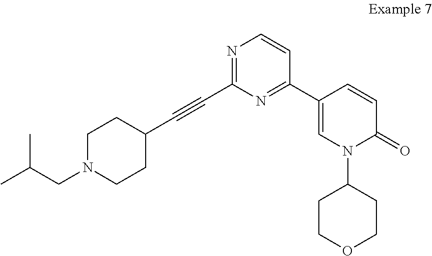 [SMILES: CC(C)CN1CCC(C#Cc2nccc(-c3ccc(=O)n(C4CCOCC4)c3)n2)CC1]

### Step 1. Synthesis of tert-butyl 4-((4-(6-oxo-1-(tetrahydro-2H-pyran-4-yl)-1,6-dihydropyridin-3-yl)pyrimidin-2-yl)ethynyl)piperidine-1-carboxylate

A mixture of compound Int-3 (180 mg, 0.617 mmol), tert-butyl 4-ethynylpiperidine-1-carboxylate (258 mg, 1.23 mmol), Pd(CH₃CN)₂Cl₂ (16 mg, 0.062 mmol), X-Phos (59 mg, 0.12 mmol) and Cs₂CO₃ (603 mg, 1.85 mmol) in CH₃CN (5 mL) was degassed and purged with N₂ for 3 times, then the mixture was stirred at 80° C. for 2 hours under N₂ atmosphere. The reaction mixture was concentrated and the residue was purified by silica gel column (0% to 90% EtOAc in PE) to give tert-butyl 4-((4-(6-oxo-1-(tetrahydro-2H-pyran-4-yl)-1,6-dihydropyridin-3-yl)pyrimidin-2-yl)ethynyl)piperidine-1-carboxylate (125 mg, yield: 37%) as a yellow gum.

### Step 2. Synthesis of 5-(2-(piperidin-4-ylethynyl)pyrimidin-4-yl)-1-(tetrahydro-2H-pyran-4-yl)pyridin-2(1H)-one

To a solution of tert-butyl 4-((4-(6-oxo-1-(tetrahydro-2H-pyran-4-yl)-1,6-dihydropyridin-3-yl)pyrimidin-2-yl)ethynyl)piperidine-1-carboxylate (100 mg, 0.215 mmol) in DCM (4 mL) was added TFA (2 mL). The mixture was stirred at 25° C. for 1 hour. The reaction mixture was concentrated to give 5-(2-(piperidin-4-ylethynyl)pyrimidin-4-yl)-1-(tetrahydro-2H-pyran-4-yl)pyridin-2(1H)-one (80 mg, crude, TFA salt) as a yellow gum.

### Step 3. Synthesis of 5-(2-((1-isobutylpiperidin-4-yl)ethynyl)pyrimidin-4-yl)-1-(tetrahydro-2H-pyran-4-yl)pyridin-2(1H)-one

To a solution of 5-(2-(piperidin-4-ylethynyl)pyrimidin-4-yl)-1-(tetrahydro-2H-pyran-4-yl)pyridin-2(1H)-one (80 mg, crude, TFA salt) in MeOH (3 mL) was added DIPEA (28 mg, 0.22 mmol). The mixture was stirred at 25° C. for 0.5 hour. Then HOAc (13 mg, 0.22 mmol) and 2-methylpropanal (79 mg, 1.1 mmol) were added to the mixture and stirred at 25° C. for 0.5 hour. NaBH₃CN (41 mg, 0.66 mmol) was added and the mixture was stirred at 25° C. for another 1 hour. The reaction mixture was concentrated and the residue was purified by prep-HPLC (0.05% NH₄HCO₃ as an additive), then lyophilized to afford the title compound (24.16 mg, yield: 21%) as a yellow solid.

¹H NMR (400 MHz, DMSO-d₆) δ 0.86 (6H, d, J=6.8 Hz), 1.61-1.70 (2H, m), 1.71-1.80 (3H, m), 1.86-1.94 (2H, m), 2.00-2.05 (2H, m), 2.06-2.18 (4H, m), 2.65-2.72 (3H, m), 3.45-3.55 (2H, m), 3.96-4.09 (2H, m), 4.88-5.05 (1H, m), 6.58 (1H, d, J=9.6 Hz), 8.05 (1H, d, J=5.6 Hz), 8.19 (1H, dd, J=9.6, 2.4 Hz), 8.60 (1H, d, J=2.4 Hz), 8.71 (1H, d, J=5.6 Hz).

### Example 8

### 1-(tetrahydro-2H-pyran-4-yl)-5-(2-((tetrahydro-2H-pyran-4-yl)ethynyl)pyrimidin-4-yl)pyridin-2(1H-one

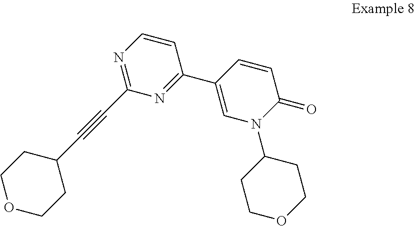 [SMILES: O=c1ccc(-c2ccnc(C#CC3CCOCC3)n2)cn1C1CCOCC1]

A mixture of compound Int-3 (120 mg, 0.411 mmol), Pd(CH₃CN)₂Cl₂ (11 mg, 0.041 mmol), Cs₂CO₃ (402 mg, 1.23 mmol) and X-Phos (39 mg, 0.082 mmol) in CH₃CN (4 mL) was degassed and purged with N₂ for 3 times, then 4-ethynyltetrahydropyran (91 mg, 0.82 mmol) was added to the mixture under N₂ atmosphere and the mixture was stirred at 80° C. for 2 hours under N₂ atmosphere. The reaction mixture was concentrated and the residue was purified by silica gel column (0% to 100% EtOAc in PE), then further purified by prep-HPLC (0.05% NH₄HCO₃ as an additive) and lyophilized to afford the title compound (41.75 mg, yield: 22%) as a white solid.

¹H NMR (400 MHz, DMSO-d₆) δ 1.60-1.71 (2H, m), 1.71-1.80 (2H, m), 1.83-1.95 (2H, m), 2.03-2.17 (2H, m), 2.89-3.08 (1H, m), 3.42-3.55 (4H, m), 3.77-3.88 (2H, m), 3.98-4.07 (2H, m), 4.91-5.03 (1H, m), 6.58 (1H, d, J=9.2 Hz), 8.07 (1H, d, J=5.6 Hz), 8.20 (1H, dd, J=9.6, 2.4 Hz), 8.61 (1H, d, J=2.4 Hz), 8.72 (1H, d, J=5.2 Hz).

The following compound was synthesized analogously to Example 8

| Example | | | |
|---|---|---|---|
| No. | Structure | Name | ¹H NMR (400 MHz) |
| 10 | 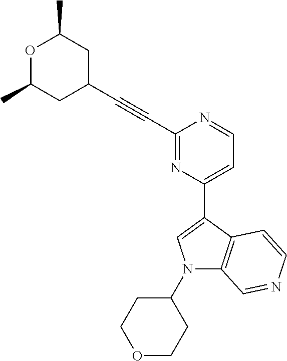 [SMILES: C[C@@H]1CC(C#Cc2nccc(-c3cn(C4CCOCC4)c4cnccc34)n2)C[C@H](C)O1] | 3-(2-(((2R,6S)-2,6- dimethyltetrahydro- 2H-pyran-4- yl)ethynyl)pyrimidin- 4-yl)-1-(tetrahydro- 2H-pyran-4-yl)-1H- pyrrolo[2,3- c]pyridine | DMSO-d₆; δ 1.13 (6H, d, J = 6.0 Hz), 1.20-1.26 (2H, m), 1.93-1.99 (2H, m), 2.01-2.15 (4H, m), 2.88-2.98 (1H, m), 3.48-3.52 (2H, m), 3.58-3.64 (2H, m), 4.00-4.06 (2H, m), 4.86-4.97 (1H, m), 7.89 (1H, d, J = 5.6 Hz), 8.34 (2H, s), 8.62 (1H, d, J = 5.6 Hz), 8.81 (1H, s), 9.12 (1H, s). |

### Example 9

### 1-(tetrahydro-2H-pyran-4-yl)-3-(2-((tetrahydro-2H-pyran-4-yl)ethynyl)pyrimidin-4-yl)-1H-pyrrolo[2,3-c]pyridine

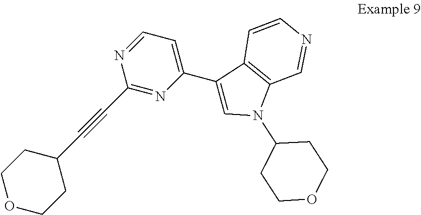 [SMILES: C(#CC1CCOCC1)c1nccc(-c2cn(C3CCOCC3)c3cnccc23)n1]

### Step 1. Synthesis of 3-bromo-1-(tetrahydro-2H-pyran-4-yl)-1H-pyrrolo[2,3-c]pyridine

To a solution of 3-bromo-1H-pyrrolo[2,3-c]pyridine (1.00 g, 5.08 mmol), tetrahydropyran-4-ol (1.04 g, 10.2 mmol) and PPh₃ (2.66 g, 10.2 mmol) in THF (10 mL) was added DIAD (2.05 g, 10.2 mmol) at 0° C., then the mixture was stirred at 60° C. for 16 hours under N₂ atmosphere. The reaction mixture was concentrated and the residue was diluted with water (20 mL), then extracted with EtOAc (30 mL×3). The combined organic layer was dried over anhydrous Na₂SO₄, filtered and concentrated. The residue was purified by silica gel column (DCM/MeOH=10/1) to afford 3-bromo-1-(tetrahydro-2H-pyran-4-yl)-1H-pyrrolo[2,3-c]pyridine (360 mg, yield: 25%) as an off-white solid.

¹H NMR (400 MHz, DMSO-d₆) δ 1.92-2.10 (4H, m), 3.52-3.60 (2H, m), 3.95-4.05 (2H, m), 4.81-4.91 (1H, m), 7.40 (1H, d, J=4.8 Hz), 8.04 (1H, d, J=1.6 Hz), 8.23 (1H, dd, J=5.2, 1.6 Hz), 9.06 (1H, s).

### Step 2. Synthesis of 1-(tetrahydro-2H-pyran-4-yl)-3-(4,4,5,5-tetramethyl-1,3,2-dioxaborolan-2-yl)-1H-pyrrolo[2,3-c]pyridine

A mixture of 3-bromo-1-(tetrahydro-2H-pyran-4-yl)-1H-pyrrolo[2,3-c]pyridine (260 mg, 0.925 mmol), Bis-Pin (282 mg, 1.11 mmol), KOAc (182 mg, 1.85 mmol), Pd(OAc)₂ (21 mg, 0.09 mmol) and PCy₃ (52 mg, 0.19 mmol) in dioxane (3 mL) was degassed and purged with N₂ for 3 times, then the mixture was stirred at 90° C. for 14 hours under N₂ atmosphere. The reaction mixture was concentrated and the residue was diluted with water (20 mL) and extracted with DCM (20 mL×3). The combined organic layer was dried over anhydrous Na₂SO₄, filtered and concentrated. The residue was purified by silica gel column (DCM/MeOH=10/1) to afford 1-(tetrahydro-2H-pyran-4-yl)-3-(4,4,5,5-tetramethyl-1,3,2-dioxaborolan-2-yl)-1H-pyrrolo[2,3-c]pyridine (240 mg, yield: 79%) as brown oil.

¹H NMR (400 MHz, DMSO-d₆) δ 1.30 (12H, s), 1.72-1.82 (2H, m), 2.02-2.13 (2H, m), 3.55-3.62 (2H, m), 4.00-4.06 (2H, m), 4.75-4.84 (1H, m), 7.71 (1H, d, J=5.6 Hz), 8.02 (1H, s), 8.19 (1H, d, J=5.2 Hz), 9.00 (1H, s).

### Step 3. Synthesis of 3-(2-chloropyrimidin-4-yl)-1-(tetrahydro-2H-pyran-4-yl)-1H-pyrrolo[2,3-c]pyridine

A mixture of 1-(tetrahydro-2H-pyran-4-yl)-3-(4,4,5,5-tetramethyl-1,3,2-dioxaborolan-2-yl)-1H-pyrrolo[2,3-c]pyridine (180 mg, 0.548 mmol), 4-bromo-2-chloropyrimidine (88 mg, 0.46 mmol), Pd(dppf)Cl₂ (33 mg, 0.05 mmol) and Na₂CO₃ (97 mg, 0.91 mmol) in dioxane (2 mL) and H₂O (0.3 mL) was degassed and purged with N₂ for 3 times, then the mixture was stirred at 90° C. for 1 hour under N₂ atmosphere. The reaction mixture was concentrated and the residue was purified by silica gel column (PE/EtOAc=0/1) to afford 3-(2-chloropyrimidin-4-yl)-1-(tetrahydro-2H-pyran-4-yl)-1H-pyrrolo[2,3-c]pyridine (140 mg, yield: 75%) as brown gum.

### Step 4. Synthesis of 1-(tetrahydro-2H-pyran-4-yl)-3-(2-((tetrahydro-2H-pyran-4-yl)ethynyl)pyrimidin-4-yl)-1H-pyrrolo[2,3-c]pyridine

A mixture of 3-(2-chloropyrimidin-4-yl)-1-(tetrahydro-2H-pyran-4-yl)-1H-pyrrolo[2,3-c]pyridine (100 mg, 0.318 mmol), 4-ethynyltetrahydro-2H-pyran (70 mg, 0.64 mmol), Pd(CH₃CN)₂Cl₂ (8 mg, 0.03 mmol), XPhos (30 mg, 0.06 mmol) and Cs₂CO₃ (311 mg, 0.953 mmol) in CH₃CN (2 mL) was degassed and purged with N₂ for 3 times, then the mixture was stirred at 80° C. for 2 hours under N₂ atmosphere. The mixture was concentrated and the residue was purified by silica gel column (DCM/MeOH=10/1), then further purified by prep-HPLC (0.225% FA as an additive) and lyophilized to afford the title compound (3.53 mg, yield: 3%) as a yellow solid.

¹H NMR (400 MHz, DMSO-d₆) δ 1.63-1.74 (2H, m), 1.87-1.96 (2H, m), 2.03-2.15 (4H, m), 2.97-3.05 (1H, m), 3.46-3.51 (2H, m), 3.58-3.64 (2H, m), 3.82-3.88 (2H, m), 4.00-4.05 (2H, m), 4.90-5.00 (1H, m), 7.91 (1H, d, J=5.6 Hz), 8.30-8.38 (2H, m), 8.63 (1H, d, J=5.6 Hz), 8.83 (1H, s), 9.13 (1H, s).

Examples 11-24 were prepared analogously to the examples described above.

| Ex- | |
|---|---|
| ample | |
| No. | Structure |
| 11 | 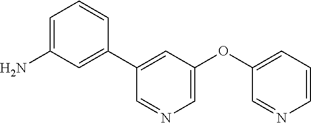 [SMILES: Nc1cccc(-c2cncc(Oc3cccnc3)c2)c1] |
| 12 | 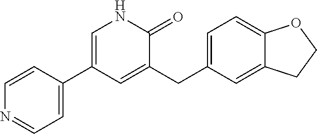 [SMILES: O=c1[nH]cc(-c2ccncc2)cc1Cc1ccc2c(c1)CCO2] |
| 13 | 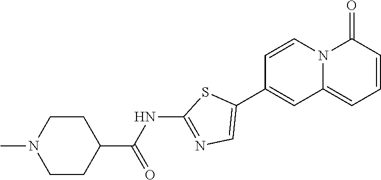 [SMILES: CN1CCC(C(=O)Nc2ncc(-c3ccn4c(=O)cccc4c3)s2)CC1] |
| 14 | 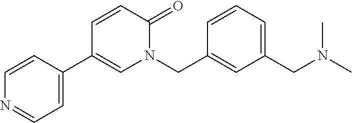 [SMILES: CN(C)Cc1cccc(Cn2cc(-c3ccncc3)ccc2=O)c1] |
| 15 | 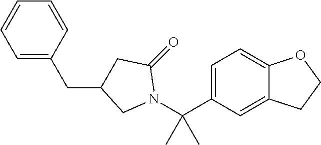 [SMILES: CC(C)(c1ccc2c(c1)CCO2)N1CC(Cc2ccccc2)CC1=O] |
| 16 | 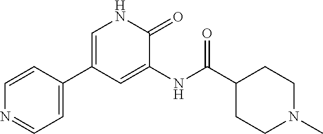 [SMILES: CN1CCC(C(=O)Nc2cc(-c3ccncc3)c[nH]c2=O)CC1] |
| 17 | 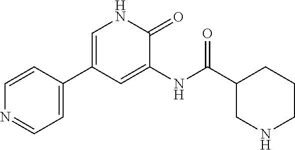 [SMILES: O=C(Nc1cc(-c2ccncc2)c[nH]c1=O)C1CCCNC1] |
| 18 | 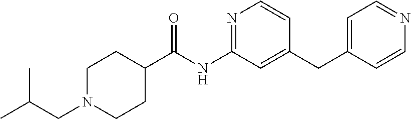 [SMILES: CC(C)CN1CCC(C(=O)Nc2cc(Cc3ccncc3)ccn2)CC1] |
| 19 | 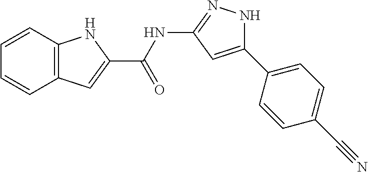 [SMILES: N#Cc1ccc(-c2cc(NC(=O)c3cc4ccccc4[nH]3)n[nH]2)cc1] |
| 20 | 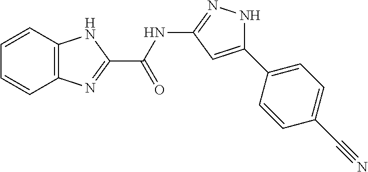 [SMILES: N#Cc1ccc(-c2cc(NC(=O)c3nc4ccccc4[nH]3)n[nH]2)cc1] |
| 21 | 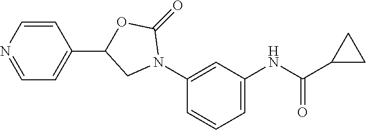 [SMILES: O=C(Nc1cccc(N2CC(c3ccncc3)OC2=O)c1)C1CC1] |
| 22 | 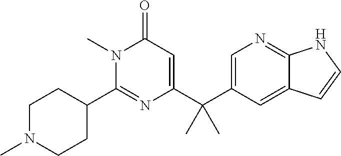 [SMILES: CN1CCC(c2nc(C(C)(C)c3cnc4[nH]ccc4c3)cc(=O)n2C)CC1] |
| 23 | 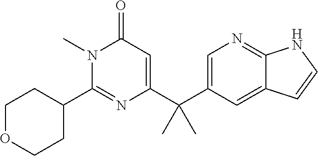 [SMILES: Cn1c(C2CCOCC2)nc(C(C)(C)c2cnc3[nH]ccc3c2)cc1=O] |
| 24 | 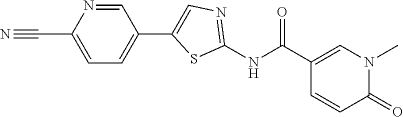 [SMILES: Cn1cc(C(=O)Nc2ncc(-c3ccc(C#N)nc3)s2)ccc1=O] |

### Example 25

### N-(2-oxaspiro[3.3]heptan-6-yl)-4-(1-(tetrahydro-2H-pyran-4-yl)-1H-pyrrolo[2,3-c]pyridin-3-yl)pyridin-2-amine

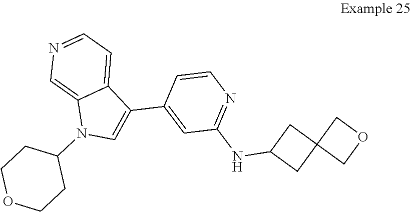 [SMILES: c1cc2c(-c3ccnc(NC4CC5(COC5)C4)c3)cn(C3CCOCC3)c2cn1]

### Step 1: Synthesis of (1-(Tetrahydro-2H-pyran-4-yl)-1H-pyrrolo[2,3-c]pyridin-3-yl)boronic acid

To a solution of 3-bromo-1-(tetrahydro-2H-pyran-4-yl)-1H-pyrrolo[2,3-c]pyridine (800 mg, 2.85 mmol) in THF (19 mL) at −78° C. was added nBuLi (2.5 M in hexanes, 1.71 mL, 4.27 mmol). After 10 minutes at −78° C., triisopropyl borate (0.98 mL, 4.27 mmol) was added dropwise. The reaction was allowed to warm to room temperature and stirred for one hour. 2 N aqueous HCl was added and the mixture stirred for 2 hours. The solution was brought to pH=8 with saturated aqueous NaHCO₃ and extracted with EtOAc (2×30 mL) and 3:1 CHCl₃:IPA (2×30 mL). The combined organics were dried over anhydrous Na₂SO₄, filtered, and concentrated in vacuo to afford 527 mg (75%) of (1-(Tetrahydro-2H-pyran-4-yl)-1H-pyrrolo[2,3-c]pyridin-3-yl)boronic acid as a yellow oil, which was used without further purification. LCMS: RT=0.40 min; ES-MS [M+1]⁺:247.3.

### Step 2: Synthesis of 4-Bromo-N-(2-oxaspiro[3.3]heptan-6-yl)pyridin-2-amine

To a solution of 4-bromo-2-fluoropyridine (100 mg, 0.57 mmol) and 2-oxaspiro[3.3]heptan-6-amine hydrochloride (128 mg, 0.85 mmol) in DMSO (1.1 mL) was added Cs₂CO₃ (931 mg, 2.84 mmol) and the reaction heated to 100° C. for 2 hours. Upon cooling to room temperature, water (10 mL) was added and the reaction extracted with EtOAc (3×10 mL). The combined organic layers were washed with brine, dried over anhydrous Na₂SO₄, filtered and concentrated in vacuo to afford 101.6 mg (66%) of 4-Bromo-N-(2-oxaspiro[3.3]heptan-6-yl)pyridin-2-amine as a brown oil, which was used without further purification. LCMS: RT=0.69 min; ES-MS [M+1]⁺:269.1/271.1.

### Step 3: Synthesis of N-(2-Oxaspiro[3.3]heptan-6-yl)-4-(1-(tetrahydro-2H-pyran-4-yl)-1H-pyrrolo[2,3-c]pyridin-3-yl)pyridin-2-amine

A solution of (1-(Tetrahydro-2H-pyran-4-yl)-1H-pyrrolo[2,3-c]pyridin-3-yl)boronic acid (35 mg, 0.1 mmol), 4-Bromo-N-(2-oxaspiro[3.3]heptan-6-yl)pyridin-2-amine (27 mg, 0.1 mmol), Pd(dppf)Cl₂ (7 mg, 0.01 mmol), and Na₂CO₃ (32 mg, 0.3 mmol) in 1,4-dioxane (1 mL) and water (0.1 mL) was degassed and purged with N₂3×. The reaction mixture was stirred at 85° C. for 2 hours. Water was added (2 mL) and the reaction extracted with DCM (3×5 mL). The combined organic layers were dried over anhydrous Na₂SO₄, filtered and concentrated in vacuo. The resulting residue was dissolved in DMSO (2 mL) and purified by prep HPLC (20-50% CH₃CN/0.05% aqueous NH₄OH over 10 min). Fractions containing the desired product were concentrated to afford 5.8 mg (15%) of N-(2-Oxaspiro[3.3]heptan-6-yl)-4-(1-(tetrahydro-2H-pyran-4-yl)-1H-pyrrolo[2,3-c]pyridin-3-yl)pyridin-2-amine as a white solid. LCMS: RT=0.65 min; ES-MS [M+1]⁺: 391.5; ¹H NMR (400 MHz, DMSO) δ 9.08 (d, J=1.0 Hz, 1H), 8.29 (s, 1H), 8.25 (d, J=5.6 Hz, 1H), 7.95 (d, J=5.4 Hz, 1H), 7.85 (dd, J=5.6, 1.1 Hz, 1H), 6.88 (dd, J=5.4, 1.5 Hz, 1H), 6.79 (d, J=1.4 Hz, 1H), 6.68 (d, J=7.0 Hz, 1H), 4.87 (tt, J=11.8, 4.2 Hz, 1H), 4.65 (s, 2H), 4.52 (s, 2H), 4.17-4.07 (m, 1H), 4.03 (dd, J=11.5, 4.3 Hz, 2H), 3.65-3.55 (m, 2H), 2.64 (ddd, J=10.1, 7.6, 3.0 Hz, 2H), 2.20-1.91 (m, 6H).

### Example 26

### 2,2,6,6-tetramethyl-N-(5-(2-methyl-2H-indazol-5-yl)thiazol-2-yl)tetrahydro-2H-pyran-4-carboxamide

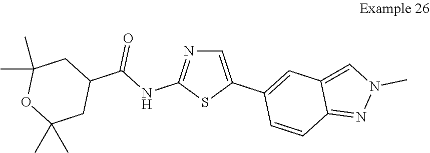

### Step 1: Synthesis of (E)-5-(2-ethoxyvinyl)-2-methyl-2H-indazole

To a microwave vial (20 mL) was added 5-bromo-2-methyl-2H-indazole (0.70 g, 3.32 mmol,), (E)-2-(2-ethoxyvinyl)-4,4,5,5-tetramethyl-1,3,2-dioxaborolane (854 mg, 4.31 mmol), Na₂CO₃ (1.07 g, 9.95 mmol), Pd(dppf)Cl₂ (243 mg, 0.33 mmol) followed by 1,4-dioxane (10 mL) and water (3 mL). The mixture was purged with nitrogen 3 times. The reaction was heated to 90° C. After 16 h, the reaction was cooled to room temperature, diluted with EtOAc, and filtered through a pad of Celite. The filtrate was concentrated under reduced pressure to give the crude. Crude was purified by flash silica gel chromatography (0-100% EtOAc/hexanes) to give (E)-5-(2-ethoxyvinyl)-2-methyl-2H-indazole (533 mg, 80%). LCMS, RT=0.56 min, ES-MS [M+H]⁺=203.1.

### Step 2: Synthesis of 5-(2-methyl-2H-indazol-5-yl)thiazol-2-amine

To a solution of (E)-5-(2-ethoxyvinyl)-2-methyl-2H-indazole (539 mg, 2.66 mmol) in a mixture of 1,4-dioxane (10 mL) and water (10 mL) was added N-Bromosuccinimide (521 mg, 2.93 mmol) at 0° C. After stirring 30 min at 0° C., thiourea (223 mg, 2.93 mmol) was added. The reaction was then heated to 100° C. After 1 h, the reaction was cooled to RT and concentrated. The residue was diluted with sat. aq. NaHCO₃, and extracted with CH₃Cl/IPA (3:1) 5 times. The combined extracts were dried (Na₂SO₄), filtered and concentrated to give the crude. Crude was purified by flash silica gel chromatography (0-10% MeOH/DCM) to give 5-(2-methyl-2H-indazol-5-yl)thiazol-2-amine (570.6 mg, 93%). LCMS, RT=0.50 min, ES-MS [M+H]⁺=231.1.

### Step 3: Synthesis of 2,2,6,6-tetramethyl-N-(5-(2-methyl-2H-indazol-5-yl)thiazol-2-yl)tetrahydro-2H-pyran-4-carboxamide

To a mixture of 5-(2-methyl-2H-indazol-5-yl)thiazol-2-amine (15 mg, 0.065 mmol), 2,2,6,6-tetramethyltetrahydro-2H-pyran-4-carboxylic acid (18 mg, 0.098 mmol), EDCI (25 mg, 0.13 mmol), HOBt (20 mg, 0.13 mmol), DMAP (8 mg, 0.065 mmol) and N,N-diisopropylethylamine (34 μL, 0.20 mmol) was added DMF (1 mL). The reaction was then heated to 70° C. After 16 h, the reaction was diluted with DMSO (1 mL), filtered through a syringe filter to give the crude. Crude product was purified using prep HPLC (5-95% ACN/0.1% aqueous TFA over 10 min). Fractions containing desired product were basified with sat. NaHCO₃ then extracted with 3:1 chloroform/IPA (3×). The combined organics were passed through a phase separator and the solvents were concentrated to give 2,2,6,6-tetramethyl-N-(5-(2-methyl-2H-indazol-5-yl)thiazol-2-yl)tetrahydro-2H-pyran-4-carboxamide (10.1 mg, 39). ¹H NMR (400 MHz, DMSO) δ 8.34 (s, 1H), 7.85 (dd, J=1.7, 0.9 Hz, 1H), 7.82 (s, 1H), 7.65-7.60 (m, 1H), 7.55 (dd, J=9.0, 1.7 Hz, 1H), 4.16 (s, 3H), 3.12 (tt, J=12.6, 3.3 Hz, 1H), 1.72 (dd, J=13.0, 3.2 Hz, 2H), 1.38 (t, J=12.7 Hz, 2H), 1.24 (s, 6H), 1.14 (s, 6H); LCMS, RT=0.73 min, ES-MS [M+H]⁺=399.1.

The following compound was synthesized analogously to Example 26

| Example | | | ¹H NMR (400 MHz); |
|---|---|---|---|
| No. | Structure | Name | ES-MS |
| 27 | 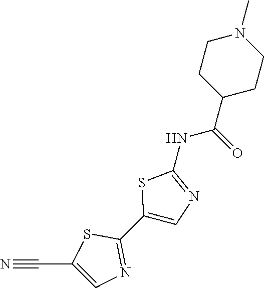 [SMILES: CN1CCC(C(=O)Nc2ncc(-c3ncc(C#N)s3)s2)CC1] | N-[5-(5-cyanothiazol-2- yl)thiazol-2-yl]-1-methyl- piperidine-4-carboxamide | ES-MS [M + 1]⁺: 333.9 |
| 28 | 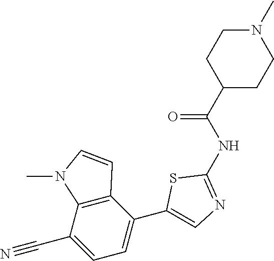 [SMILES: CN1CCC(C(=O)Nc2ncc(-c3ccc(C#N)c4c3ccn4C)s2)CC1] | N-[5-(7-cyano-1-methyl- indol-4-yl)thiazol-2-yl]-1- methyl-piperidine-4- carboxamide | ES-MS [M + 1]⁺: 380.2 |
| 29 | 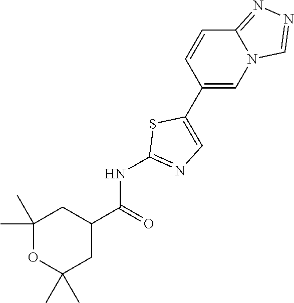 [SMILES: CC1(C)CC(C(=O)Nc2ncc(-c3ccc4nncn4c3)s2)CC(C)(C)O1] | N-(5-([1,2,4]triazolo[4,3- a]pyridin-6-yl)thiazol-2- yl)-2,2,6,6- tetramethyltetrahydro-2H- pyran-4-carboxamide | DMSO; δ 9.20 (d, J = 0.8 Hz, 1H), 8.85 (t, J = 1.4 Hz, 1H), 7.99 (s, 1H), 7.85 (dt, J = 9.6, 1.0 Hz, 1H), 7.76 (dd, J = 9.6, 1.7 Hz, 1H), 3.18- 3.06 (m, 1H), 1.74 (dd, J = 13.0, 3.2 Hz, 2H), 1.38 (t, J = 12.7 Hz, 2H), 1.24 (s, 6H), 1.14 (s, 6H); ES-MS [M + H]⁺: 386.2. |
| 30 | 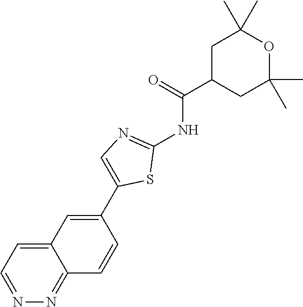 [SMILES: CC1(C)CC(C(=O)Nc2ncc(-c3ccc4nnccc4c3)s2)CC(C)(C)O1] | N-(5-(cinnolin-6-yl)thiazol- 2-yl)-2,2,6,6- tetramethyltetrahydro-2H- pyran-4-carboxamide | DMSO; δ 9.34 (d, J = 5.9 Hz, 1H), 8.45 (dt, J = 9.0, 0.8 Hz, 1H), 8.34 (dd, J = 9.0, 2.0 Hz, 1H), 8.26 (s, 1H), 8.23- 8.18 (m, 2H), 3.15 (tt, J = 12.5, 3.3 Hz, 1H), 1.75 (dd, J = 13.0, 3.2 Hz, 2H), 1.39 (t, J = 12.7 Hz, 2H), 1.24 (s, 6H), 1.15 (s, 6H); ES- MS [M + H]⁺: 397.1. |
| 31 | 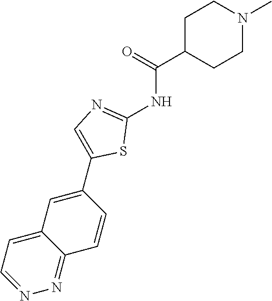 [SMILES: CN1CCC(C(=O)Nc2ncc(-c3ccc4nnccc4c3)s2)CC1] | N-(5-(cinnolin-6-yl)thiazol- 2-yl)-1-methylpiperidine-4- carboxamide | DMSO; δ 9.33 (d, J = 5.9 Hz, 1H), 8.43 (d, J = 9.0 Hz, 1H), 8.34-8.30 (m, 1H), 8.22 (s, 1H), 8.20-8.13 (m, 2H), 2.87-2.76 (m, 2H), 2.48-2.40 (m, 1H), 2.16 (s, 3H), 1.93-1.76 (m, 4H), 1.73-1.59 (m, 2H); ES-MS [M + H]⁺: 354.1. |
| 32 | 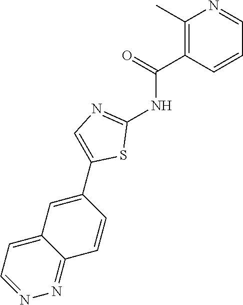 [SMILES: Cc1ncccc1C(=O)Nc1ncc(-c2ccc3nnccc3c2)s1] | N-(5-(cinnolin-6-yl)thiazol- 2-yl)-2-methylnicotinamide | DMSO; δ 9.34 (d, J = 5.8 Hz, 1H), 8.58 (dd, J = 4.9, 1.7 Hz, 1H), 8.45 (d, J = 9.0 Hz, 1H), 8.38 (dd, J = 9.0, 2.0 Hz, 1H), 8.31 (s, 1H), 8.25 (d, J = 1.9 Hz, 1H), 8.20 (dd, J = 6.0, 0.9 Hz, 1H), 8.04 (dd, J = 7.8, 1.8 Hz, 1H), 7.36 (dd, J = 7.8, 4.9 Hz, 1H), 2.63 (s, 3H); ES-MS [M + H]⁺: 348.1. |
| 33 | 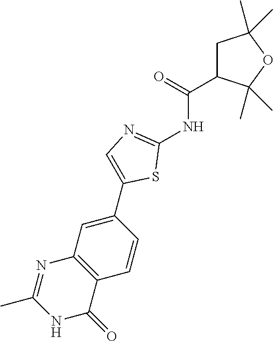 | 2,2,5,5-tetramethyl-N-[5- (2-methyl-4-oxo-3H- quinazolin-7-yl)-1,3- thiazol-2-yl]oxolane-3- carboxamide | DMSO-d6; δ 8.02 (d, J = 6.3 Hz, 1 H), 7.82 (s, 1H), 7.64 (m, 1H), 7.54 (s, 1H), 3.35-3.28 (m, 1H), 2.42-2.31 (m, 4H), 2.08-2.02 (m, 1H), 1.35 (s, 3H), 1.30 (s, 3H), 1.18 (s, 3H), 1.04 (s, 3H); ES-MS [M + 1]⁺: 413.2. |
| 34 | 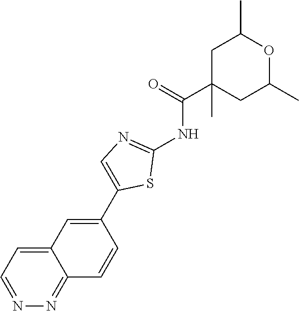 [SMILES: CC1CC(C)(C(=O)Nc2ncc(-c3ccc4nnccc4c3)s2)CC(C)O1] | N-(5-(cinnolin-6-yl)thiazol- 2-yl)-2,4,6- trimethyltetrahydro-2H- pyran-4-carboxamide | DMSO; δ 9.33 (d, J = 5.9 Hz, 1H), 8.47-8.40 (m, 1H), 8.32 (dd, J = 9.0, 2.0 Hz, 1H), 8.25 (d, J = 1.3 Hz, 1H), 8.20- 8.14 (m, 2H), 3.41 (dtt, J = 12.7, 6.5, 3.2 Hz, 2H), 2.32 (d, J = 13.5 Hz, 2H), 1.23 (s, 3H), 1.09 (s, 3H), 1.07 (s, 3 H), 1.06-0.98 (m, 2H); ES-MS [M + H]⁺ = 383.2. |
| 35 | 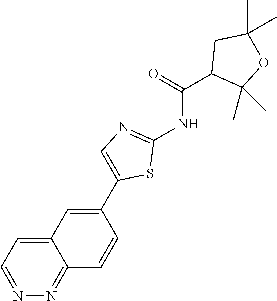 [SMILES: CC1(C)CC(C(=O)Nc2ncc(-c3ccc4nnccc4c3)s2)C(C)(C)O1] | N-(5-(cinnolin-6-yl)thiazol- 2-yl)-2,2,5,5- tetramethyltetrahydrofuran- 3-carboxamide | DMSO; δ 9.35 (d, J = 5.9 Hz, 1H), 8.45 (d, J = 9.0 Hz, 1H), 8.36 (dd, J = 9.0, 2.0 Hz, 1H), 8.28 (s, 1H), 8.26-8.20 (m, 2H), 3.31 (dd, J = 11.3, 7.1 Hz, 1H), 2.34 (t, J = 11.9 Hz, 1H), 2.05 (dd, J = 12.4, 7.2 Hz, 1H), 1.35 (s, 3H), 1.30 (s, 3H), 1.17 (s, 3H), 1.04 (s, 3H); LCMS: RT = 0.70 min, ES-MS [M + H]⁺ = 383.2. |
| 36 | 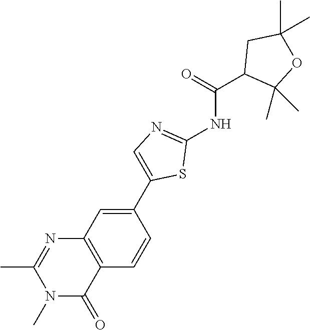 | N-(5-(2,3-dimethyl-4-oxo- 3,4-dihydroquinazolin-7- yl)thiazol-2-yl)-2,2,5,5- tetramethyltetrahydrofuran- 3-carboxamide | ES-MS [M + H]⁺: 427.2. |
| 37 | 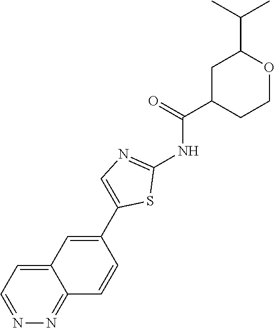 [SMILES: CC(C)C1CC(C(=O)Nc2ncc(-c3ccc4nnccc4c3)s2)CCO1] | N-(5-(cinnolin-6- yl)thiazol-2-yl)-2- isopropyltetrahydro-2H- pyran-4-carboxamide | ES-MS [M + H]⁺: 383.4. |
| 38 | 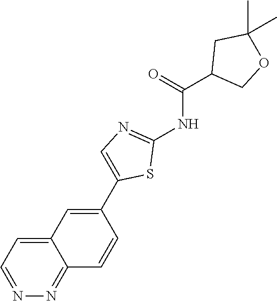 [SMILES: CC1(C)CC(C(=O)Nc2ncc(-c3ccc4nnccc4c3)s2)CO1] | N-(5-(cinnolin-6-yl)thiazol- 2-yl)-5,5- dimethyltetrahydrofuran-3- carboxamide | ES-MS [M + H]⁺: 355.1. |

Compounds of Formula (I), (II), (III), and related analogs and their associated LC-MS data are shown in the Table below. These compounds were prepared according to procedures analogous to the procedures above, with modifications where appropriate that are within the purview of one skilled in the art. Liquid Chromatography-Mass Spectrometry (LCMS) was taken on a quadruple Mass Spectrometer on Waters QDa/Acquity I-Class LCMS (Column: Phenomenex Kinetex EVO C18 (1.0×50 mm, 1.7 um)) operating in ESI (+) ionization mode. Flow Rate: 0.4 mL/min, Acquire Time: 1.5 min, Wavelength: UV215 & 254, Oven Temp.: 55° C.

TABLE 4

| | LC-MS | |
|---|---|---|
| | Retention | |
| | Time | m/z |
| Compound structure | (min) | (M + H) |
| 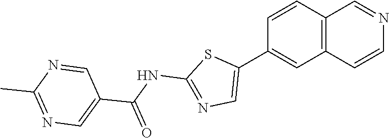 [SMILES: Cc1ncc(C(=O)Nc2ncc(-c3ccc4cnccc4c3)s2)cn1] | 0.447 | 348.1 |
| 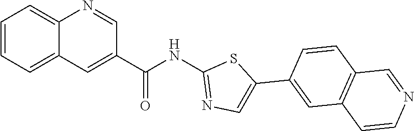 [SMILES: O=C(Nc1ncc(-c2ccc3cnccc3c2)s1)c1cnc2ccccc2c1] | 0.527 | 383.2 |
| 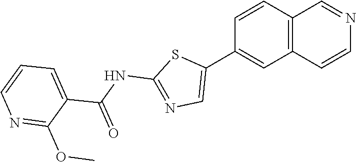 [SMILES: COc1ncccc1C(=O)Nc1ncc(-c2ccc3cnccc3c2)s1] | 0.880 | 363.3 |
| 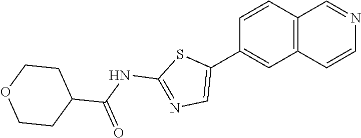 [SMILES: O=C(Nc1ncc(-c2ccc3cnccc3c2)s1)C1CCOCC1] | 0.692 | 340.2 |
| 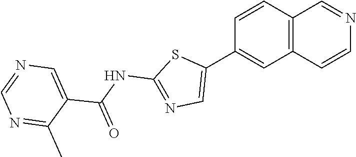 [SMILES: Cc1ncncc1C(=O)Nc1ncc(-c2ccc3cnccc3c2)s1] | 0.584 | 348.3 |
| 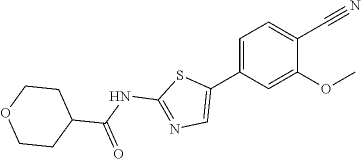 [SMILES: COc1cc(-c2cnc(NC(=O)C3CCOCC3)s2)ccc1C#N] | 0.680 | 344.2 |
| 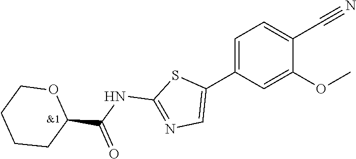 [SMILES: COc1cc(-c2cnc(NC(=O)[C@H]3CCCCO3)s2)ccc1C#N] | 0.767 | 344.2 |
| 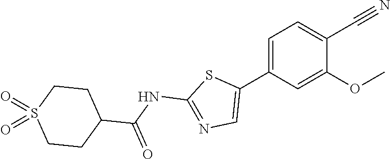 [SMILES: COc1cc(-c2cnc(NC(=O)C3CCS(=O)(=O)CC3)s2)ccc1C#N] | 0.630 | 392.2 |
| 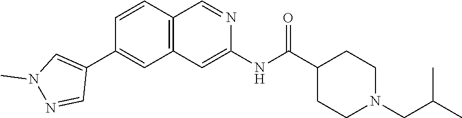 [SMILES: CC(C)CN1CCC(C(=O)Nc2cc3cc(-c4cnn(C)c4)ccc3cn2)CC1] | 0.47 | 392.2 |
| 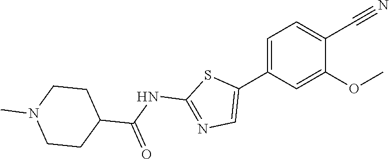 | 0.487 | 357.3 |
| 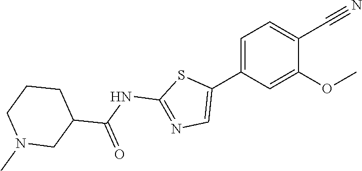 | 0.487 | 357.3 |
| 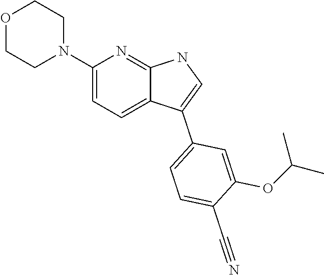 [SMILES: CC(C)Oc1cc(-c2c[nH]c3nc(N4CCOCC4)ccc23)ccc1C#N] | 0.740 | 363.3 |
| 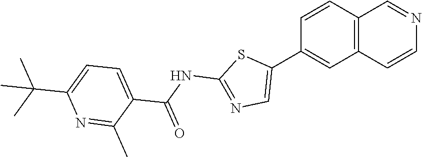 [SMILES: Cc1nc(C(C)(C)C)ccc1C(=O)Nc1ncc(-c2ccc3cnccc3c2)s1] | 1.041 | 403.4 |
| 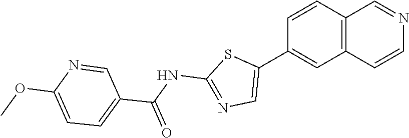 [SMILES: COc1ccc(C(=O)Nc2ncc(-c3ccc4cnccc4c3)s2)cn1] | 0.529 | 363.2 |
| 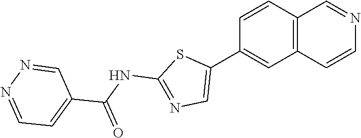 [SMILES: O=C(Nc1ncc(-c2ccc3cnccc3c2)s1)c1ccnnc1] | 0.469 | 334.2 |
| 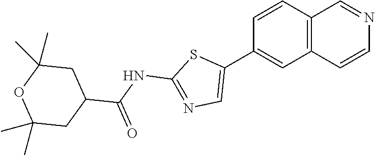 [SMILES: CC1(C)CC(C(=O)Nc2ncc(-c3ccc4cnccc4c3)s2)CC(C)(C)O1] | 0.913 | 396.5 |
| 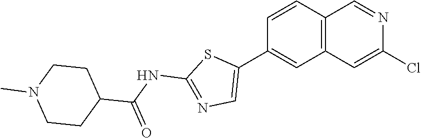 [SMILES: CN1CCC(C(=O)Nc2ncc(-c3ccc4cnc(Cl)cc4c3)s2)CC1] | 0.547 | 387.2 |
| 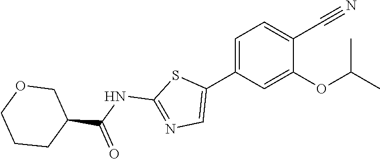 [SMILES: CC(C)Oc1cc(-c2cnc(NC(=O)[C@H]3CCCOC3)s2)ccc1C#N] | 0.78 | 372.2 |
| 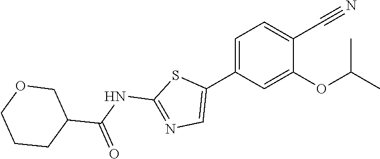 [SMILES: CC(C)Oc1cc(-c2cnc(NC(=O)C3CCCOC3)s2)ccc1C#N] | 0.78 | 372.4 |
| 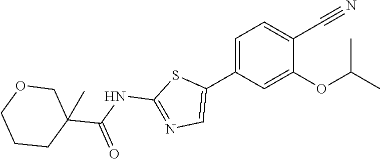 [SMILES: CC(C)Oc1cc(-c2cnc(NC(=O)C3(C)CCCOC3)s2)ccc1C#N] | 0.81 | 386.1 |
| 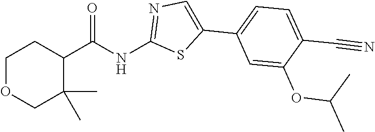 [SMILES: CC(C)Oc1cc(-c2cnc(NC(=O)C3CCOCC3(C)C)s2)ccc1C#N] | 0.80 | 400.1 |
| 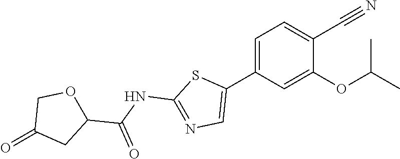 [SMILES: CC(C)Oc1cc(-c2cnc(NC(=O)C3CC(=O)CO3)s2)ccc1C#N] | 0.70 | 372.0 |
| 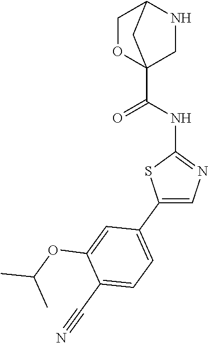 [SMILES: CC(C)Oc1cc(-c2cnc(NC(=O)C34CNC(CO3)C4)s2)ccc1C#N] | 0.55 | 385.2 |
| 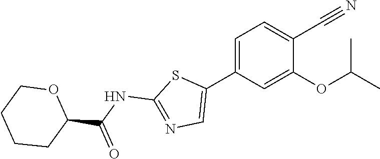 [SMILES: CC(C)Oc1cc(-c2cnc(NC(=O)[C@H]3CCCCO3)s2)ccc1C#N] | 0.84 | 372.2 |
| 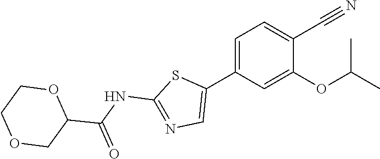 [SMILES: CC(C)Oc1cc(-c2cnc(NC(=O)C3COCCO3)s2)ccc1C#N] | 0.74 | 374.2 |
| 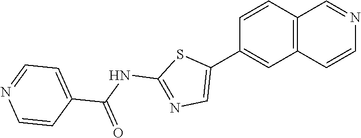 [SMILES: O=C(Nc1ncc(-c2ccc3cnccc3c2)s1)c1ccncc1] | 0.384 | 333.1 |
| 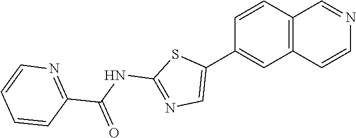 [SMILES: O=C(Nc1ncc(-c2ccc3cnccc3c2)s1)c1ccccn1] | 0.516 | 333.1 |
| 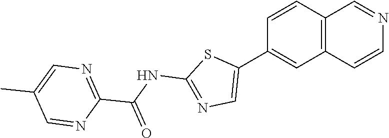 [SMILES: Cc1cnc(C(=O)Nc2ncc(-c3ccc4cnccc4c3)s2)nc1] | 0.463 | 348.1 |
| 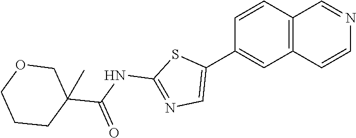 [SMILES: CC1(C(=O)Nc2ncc(-c3ccc4cnccc4c3)s2)CCCOC1] | 0.477 | 354.2 |
| 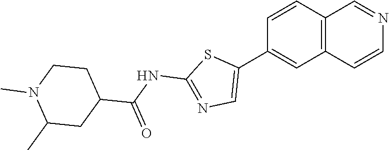 [SMILES: CC1CC(C(=O)Nc2ncc(-c3ccc4cnccc4c3)s2)CCN1C] | 0.327 | 367.2 |
| 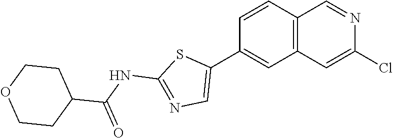 [SMILES: O=C(Nc1ncc(-c2ccc3cnc(Cl)cc3c2)s1)C1CCOCC1] | 0.755 | 374.2 |
| 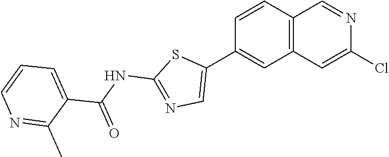 [SMILES: Cc1ncccc1C(=O)Nc1ncc(-c2ccc3cnc(Cl)cc3c2)s1] | 0.613 | 381.1 |
| 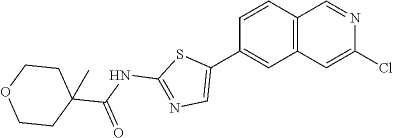 [SMILES: CC1(C(=O)Nc2ncc(-c3ccc4cnc(Cl)cc4c3)s2)CCOCC1] | 0.793 | 388.2 |
| 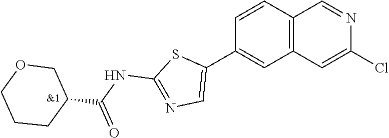 [SMILES: O=C(Nc1ncc(-c2ccc3cnc(Cl)cc3c2)s1)[C@@H]1CCCOC1] | 0.771 | 374.2 |
| 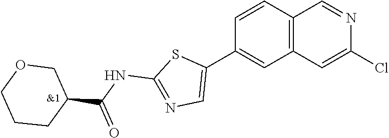 [SMILES: O=C(Nc1ncc(-c2ccc3cnc(Cl)cc3c2)s1)[C@H]1CCCOC1] | 0.771 | 374.2 |
| 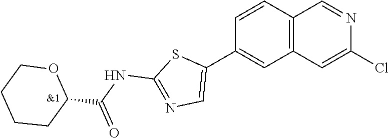 [SMILES: O=C(Nc1ncc(-c2ccc3cnc(Cl)cc3c2)s1)[C@@H]1CCCCO1] | 0.839 | 374.2 |
| 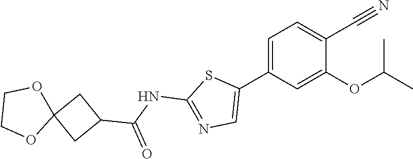 [SMILES: CC(C)Oc1cc(-c2cnc(NC(=O)C3CC4(C3)OCCO4)s2)ccc1C#N] | 0.76 | 400.2 |
| 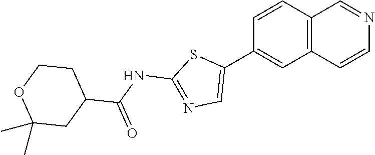 [SMILES: CC1(C)CC(C(=O)Nc2ncc(-c3ccc4cnccc4c3)s2)CCO1] | 0.504 | 368.3 |
| 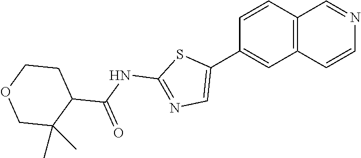 [SMILES: CC1(C)COCCC1C(=O)Nc1ncc(-c2ccc3cnccc3c2)s1] | 0.502 | 368.3 |
| 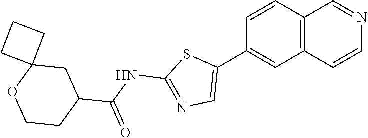 [SMILES: O=C(Nc1ncc(-c2ccc3cnccc3c2)s1)C1CCOC2(CCC2)C1] | 0.530 | 380.2 |
| 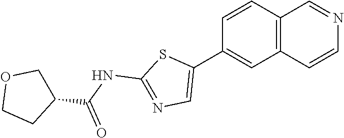 [SMILES: O=C(Nc1ncc(-c2ccc3cnccc3c2)s1)[C@@H]1CCOC1] | 0.414 | 326.1 |
| 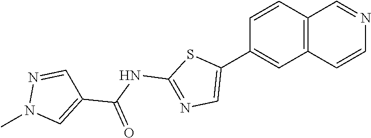 [SMILES: Cn1cc(C(=O)Nc2ncc(-c3ccc4cnccc4c3)s2)cn1] | 0.454 | 336.1 |
| 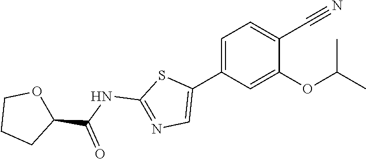 [SMILES: CC(C)Oc1cc(-c2cnc(NC(=O)[C@H]3CCCO3)s2)ccc1C#N] | 0.76 | 358.0 |
| 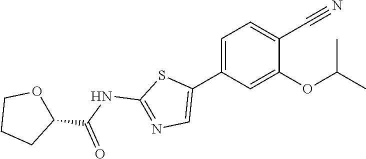 [SMILES: CC(C)Oc1cc(-c2cnc(NC(=O)[C@@H]3CCCO3)s2)ccc1C#N] | 0.76 | 358.0 |
| 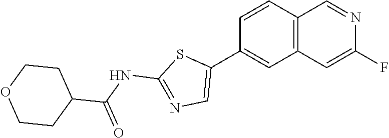 [SMILES: O=C(Nc1ncc(-c2ccc3cnc(F)cc3c2)s1)C1CCOCC1] | 0.682 | 358.1 |
| 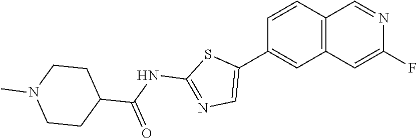 [SMILES: CN1CCC(C(=O)Nc2ncc(-c3ccc4cnc(F)cc4c3)s2)CC1] | 0.490 | 371.2 |
| 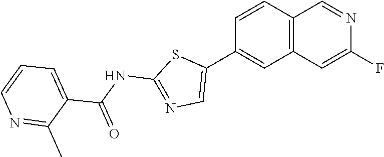 [SMILES: Cc1ncccc1C(=O)Nc1ncc(-c2ccc3cnc(F)cc3c2)s1] | 0.556 | 365.1 |
| 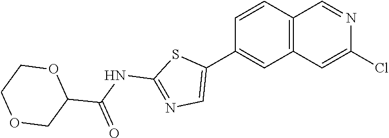 [SMILES: O=C(Nc1ncc(-c2ccc3cnc(Cl)cc3c2)s1)C1COCCO1] | 0.710 | 376.1 |
| 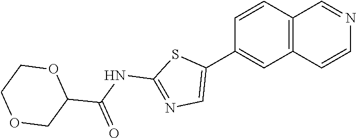 [SMILES: O=C(Nc1ncc(-c2ccc3cnccc3c2)s1)C1COCCO1] | 0.418 | 342.2 |
| 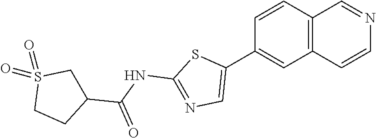 [SMILES: O=C(Nc1ncc(-c2ccc3cnccc3c2)s1)C1CCS(=O)(=O)C1] | 0.387 | 374.2 |
| 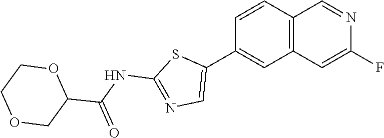 [SMILES: O=C(Nc1ncc(-c2ccc3cnc(F)cc3c2)s1)C1COCCO1] | 0.696 | 360.2 |
| 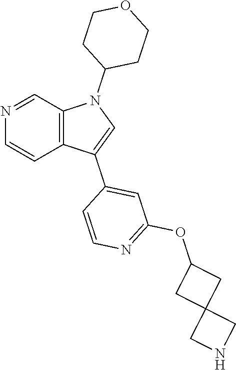 [SMILES: c1cc2c(-c3ccnc(OC4CC5(CNC5)C4)c3)cn(C3CCOCC3)c2cn1] | 0.522 | 391.5 |
| 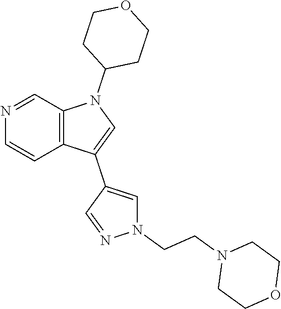 [SMILES: c1cc2c(-c3cnn(CCN4CCOCC4)c3)cn(C3CCOCC3)c2cn1] | 0.594 | 382.3 |
| 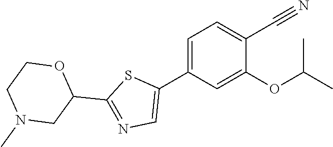 [SMILES: CC(C)Oc1cc(-c2cnc(C3CN(C)CCO3)s2)ccc1C#N] | 0.53 | 344.2 |
| 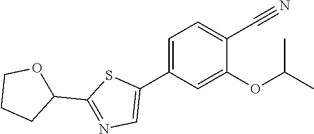 [SMILES: CC(C)Oc1cc(-c2cnc(C3CCCO3)s2)ccc1C#N] | 0.80 | 315.1 |
| 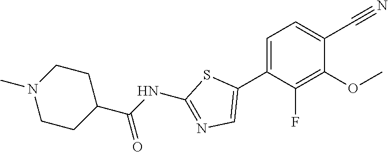 | 0.48 | 375.3 |
| 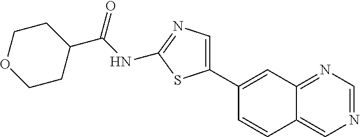 [SMILES: O=C(Nc1ncc(-c2ccc3cncnc3c2)s1)C1CCOCC1] | 0.475 | 341.1 |
| 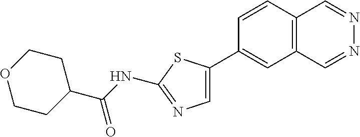 [SMILES: O=C(Nc1ncc(-c2ccc3cnncc3c2)s1)C1CCOCC1] | 0.425 | 341.1 |
| 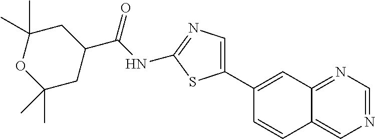 [SMILES: CC1(C)CC(C(=O)Nc2ncc(-c3ccc4cncnc4c3)s2)CC(C)(C)O1] | 0.633 | 397.2 |
| 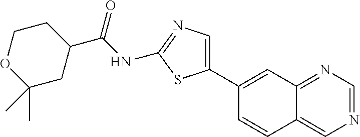 [SMILES: CC1(C)CC(C(=O)Nc2ncc(-c3ccc4cncnc4c3)s2)CCO1] | 0.543 | 369.1 |
| 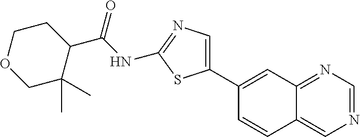 [SMILES: CC1(C)COCCC1C(=O)Nc1ncc(-c2ccc3cncnc3c2)s1] | 0.563 | 369.1 |
| 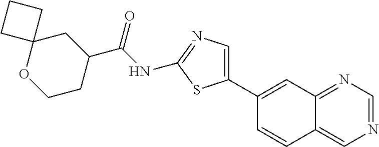 [SMILES: O=C(Nc1ncc(-c2ccc3cncnc3c2)s1)C1CCOC2(CCC2)C1] | 0.580 | 381.1 |
| 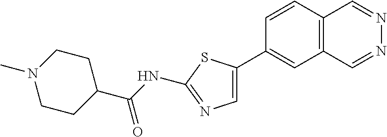 [SMILES: CN1CCC(C(=O)Nc2ncc(-c3ccc4cnncc4c3)s2)CC1] | 0.268 | 354.2 |
| 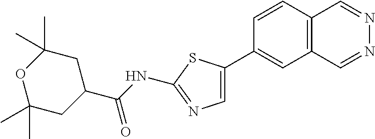 [SMILES: CC1(C)CC(C(=O)Nc2ncc(-c3ccc4cnncc4c3)s2)CC(C)(C)O1] | 0.574 | 397.2 |
| 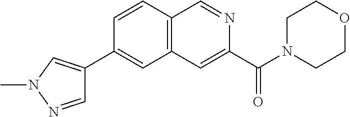 [SMILES: Cn1cc(-c2ccc3cnc(C(=O)N4CCOCC4)cc3c2)cn1] | 0.473 | 323.0 |
| 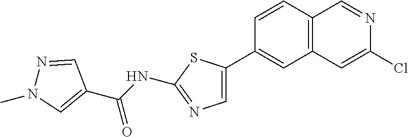 [SMILES: Cn1cc(C(=O)Nc2ncc(-c3ccc4cnc(Cl)cc4c3)s2)cn1] | 0.499 | 267.0 |
| 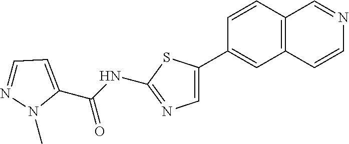 [SMILES: Cn1nccc1C(=O)Nc1ncc(-c2ccc3cnccc3c2)s1] | 0.458 | 336.1 |
| 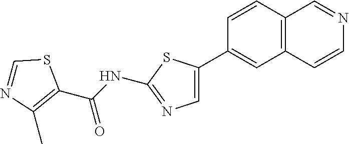 [SMILES: Cc1ncsc1C(=O)Nc1ncc(-c2ccc3cnccc3c2)s1] | 0.472 | 353.0 |
| 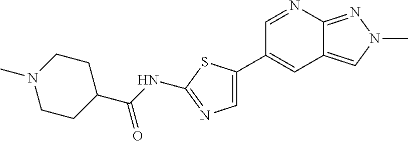 | 0.335 | 357.2 |
| 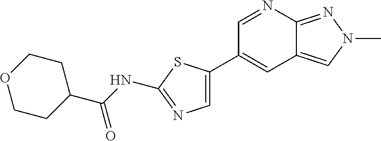 [SMILES: Cn1cc2cc(-c3cnc(NC(=O)C4CCOCC4)s3)cnc2n1] | 0.472 | 344.1 |
| 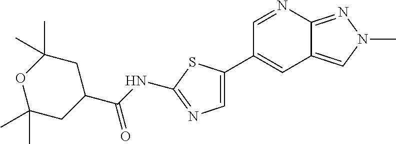 | 0.630 | 400.2 |
| 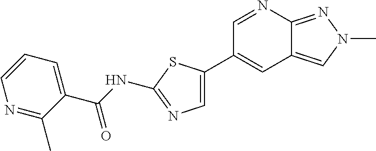 [SMILES: Cc1ncccc1C(=O)Nc1ncc(-c2cnc3nn(C)cc3c2)s1] | 0.405 | 351.0 |
| 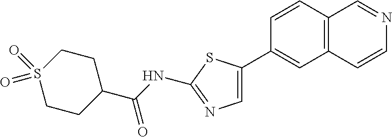 [SMILES: O=C(Nc1ncc(-c2ccc3cnccc3c2)s1)C1CCS(=O)(=O)CC1] | 0.396 | 388.0 |
| 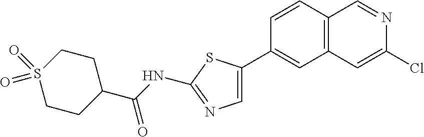 [SMILES: O=C(Nc1ncc(-c2ccc3cnc(Cl)cc3c2)s1)C1CCS(=O)(=O)CC1] | 0.673 | 422.0 |
| 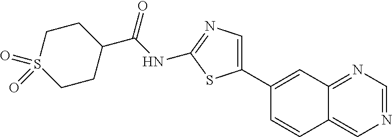 [SMILES: O=C(Nc1ncc(-c2ccc3cncnc3c2)s1)C1CCS(=O)(=O)CC1] | 0.450 | 389.0 |
| 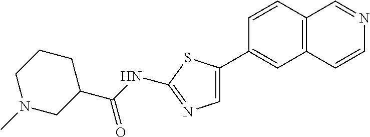 [SMILES: CN1CCCC(C(=O)Nc2ncc(-c3ccc4cnccc4c3)s2)C1] | 0.290 | 353.1 |
| 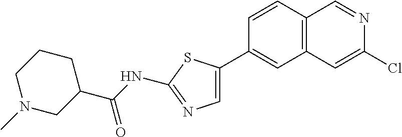 [SMILES: CN1CCCC(C(=O)Nc2ncc(-c3ccc4cnc(Cl)cc4c3)s2)C1] | 0.539 | 387.0 |
| 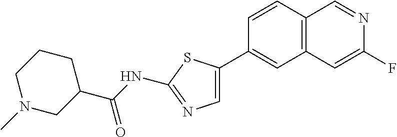 [SMILES: CN1CCCC(C(=O)Nc2ncc(-c3ccc4cnc(F)cc4c3)s2)C1] | 0.506 | 371.1 |
| 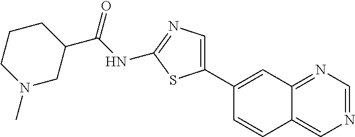 [SMILES: CN1CCCC(C(=O)Nc2ncc(-c3ccc4cncnc4c3)s2)C1] | 0.366 | 354.1 |
| 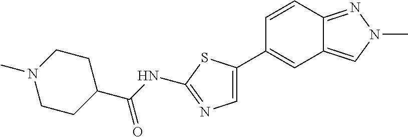 | 0.423 | 356.2 |
| 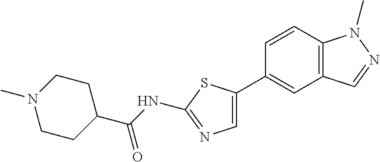 [SMILES: CN1CCC(C(=O)Nc2ncc(-c3ccc4c(cnn4C)c3)s2)CC1] | 0.448 | 356.2 |
| 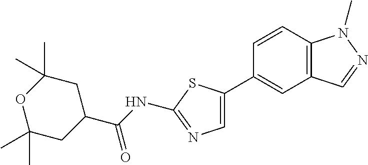 [SMILES: Cn1ncc2cc(-c3cnc(NC(=O)C4CC(C)(C)OC(C)(C)C4)s3)ccc21] | 0.768 | 399.2 |
| 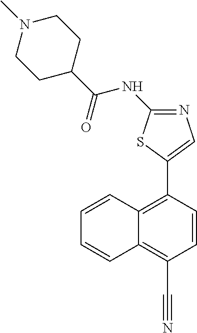 [SMILES: CN1CCC(C(=O)Nc2ncc(-c3ccc(C#N)c4ccccc34)s2)CC1] | 0.44 | 378.1 |
| 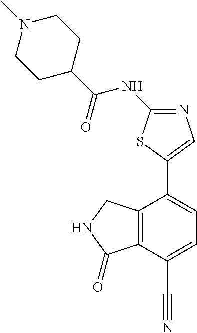 [SMILES: CN1CCC(C(=O)Nc2ncc(-c3ccc(C#N)c4c3CNC4=O)s2)CC1] | 0.38 | 382.1 |
| 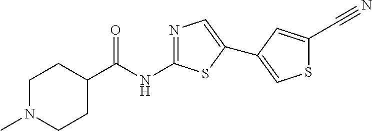 [SMILES: CN1CCC(C(=O)Nc2ncc(-c3csc(C#N)c3)s2)CC1] | 0.42 | 333.1 |
| 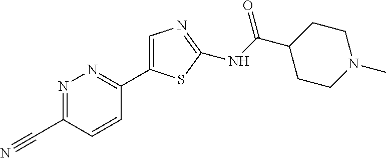 [SMILES: CN1CCC(C(=O)Nc2ncc(-c3ccc(C#N)nn3)s2)CC1] | 0.52 | 333.9 |
| 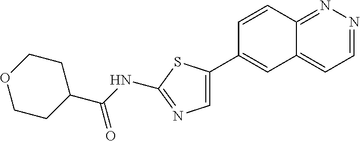 [SMILES: O=C(Nc1ncc(-c2ccc3nnccc3c2)s1)C1CCOCC1] | 0.531 | 341.1 |
| 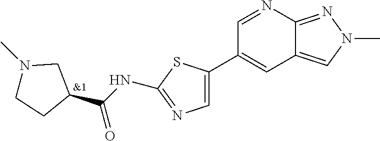 | 0.323 | 343.2 |
| 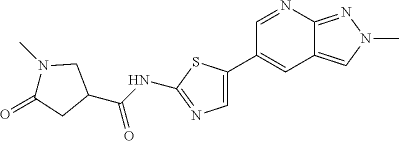 [SMILES: CN1CC(C(=O)Nc2ncc(-c3cnc4nn(C)cc4c3)s2)CC1=O] | 0.425 | 357.1 |
| 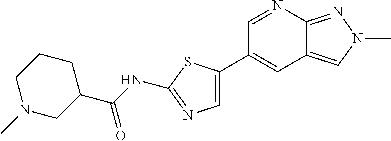 | 0.354 | 357.2 |
| 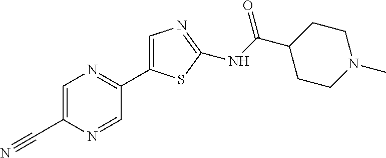 [SMILES: CN1CCC(C(=O)Nc2ncc(-c3cnc(C#N)cn3)s2)CC1] | 0.44 | 329.0 |
| 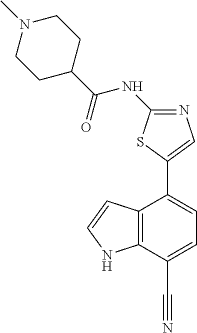 [SMILES: CN1CCC(C(=O)Nc2ncc(-c3ccc(C#N)c4[nH]ccc34)s2)CC1] | 0.59 | 366.1 |
| 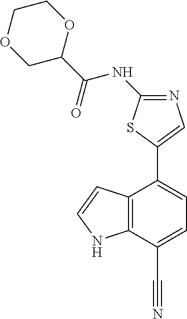 [SMILES: N#Cc1ccc(-c2cnc(NC(=O)C3COCCO3)s2)c2cc[nH]c12] | 0.63 | 355.1 |
| 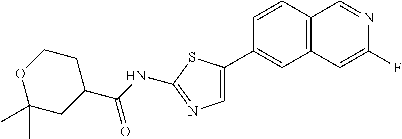 [SMILES: CC1(C)CC(C(=O)Nc2ncc(-c3ccc4cnc(F)cc4c3)s2)CCO1] | 0.743 | 386.1 |
| 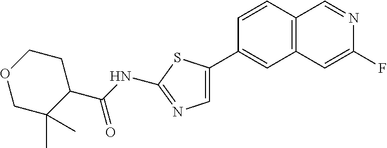 [SMILES: CC1(C)COCCC1C(=O)Nc1ncc(-c2ccc3cnc(F)cc3c2)s1] | 0.753 | 386.2 |
| 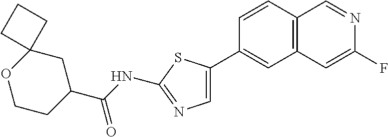 [SMILES: O=C(Nc1ncc(-c2ccc3cnc(F)cc3c2)s1)C1CCOC2(CCC2)C1] | 0.774 | 398.1 |
| 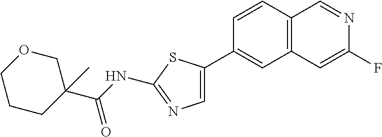 [SMILES: CC1(C(=O)Nc2ncc(-c3ccc4cnc(F)cc4c3)s2)CCCOC1] | 0.875 | 372.0 |
| 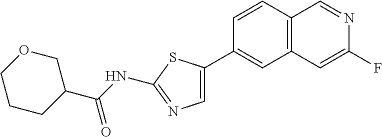 [SMILES: O=C(Nc1ncc(-c2ccc3cnc(F)cc3c2)s1)C1CCCOC1] | 0.803 | 358.0 |
| 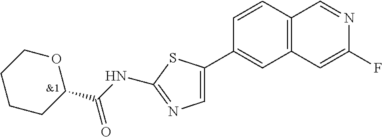 [SMILES: O=C(Nc1ncc(-c2ccc3cnc(F)cc3c2)s1)[C@@H]1CCCCO1] | 0.903 | 358.0 |
| 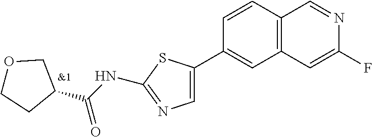 [SMILES: O=C(Nc1ncc(-c2ccc3cnc(F)cc3c2)s1)[C@@H]1CCOC1] | 0.742 | 344.0 |
| 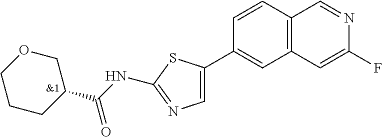 [SMILES: O=C(Nc1ncc(-c2ccc3cnc(F)cc3c2)s1)[C@@H]1CCCOC1] | 0.803 | 358.0 |
| 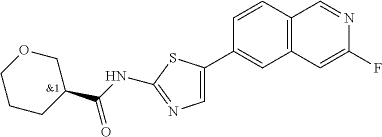 [SMILES: O=C(Nc1ncc(-c2ccc3cnc(F)cc3c2)s1)[C@H]1CCCOC1] | 0.803 | 358.0 |
| 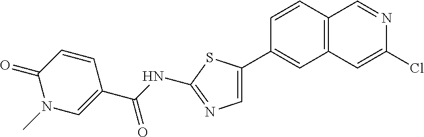 [SMILES: Cn1cc(C(=O)Nc2ncc(-c3ccc4cnc(Cl)cc4c3)s2)ccc1=O] | 0.803 | 397.0 |
| 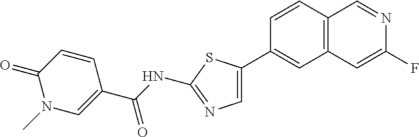 [SMILES: Cn1cc(C(=O)Nc2ncc(-c3ccc4cnc(F)cc4c3)s2)ccc1=O] | 0.803 | 380.9 |
| 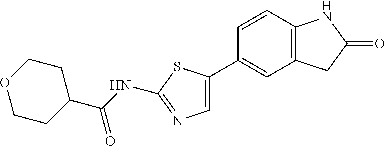 [SMILES: O=C1Cc2cc(-c3cnc(NC(=O)C4CCOCC4)s3)ccc2N1] | 0.576 | 344.0 |
| 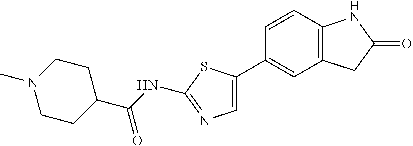 [SMILES: CN1CCC(C(=O)Nc2ncc(-c3ccc4c(c3)CC(=O)N4)s2)CC1] | 0.372 | 357.3 |
| 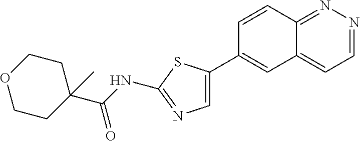 [SMILES: CC1(C(=O)Nc2ncc(-c3ccc4nnccc4c3)s2)CCOCC1] | 0.642 | 355.0 |
| 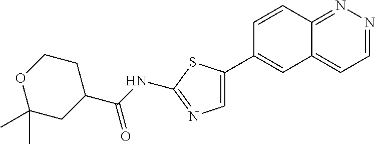 [SMILES: CC1(C)CC(C(=O)Nc2ncc(-c3ccc4nnccc4c3)s2)CCO1] | 0.671 | 369.1 |
| 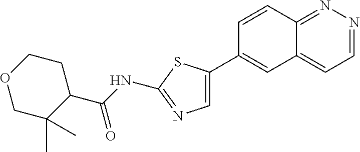 [SMILES: CC1(C)COCCC1C(=O)Nc1ncc(-c2ccc3nnccc3c2)s1] | 0.677 | 369.1 |
| 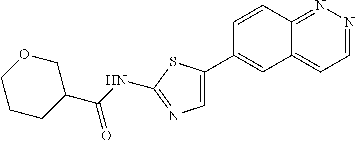 [SMILES: O=C(Nc1ncc(-c2ccc3nnccc3c2)s1)C1CCCOC1] | 0.622 | 341.0 |
| 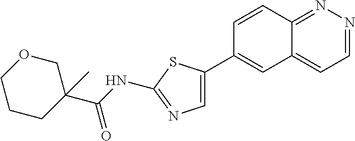 [SMILES: CC1(C(=O)Nc2ncc(-c3ccc4nnccc4c3)s2)CCCOC1] | 0.674 | 355.0 |
| 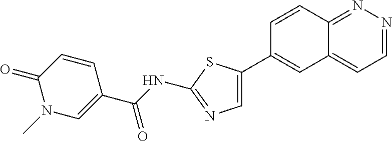 [SMILES: Cn1cc(C(=O)Nc2ncc(-c3ccc4nnccc4c3)s2)ccc1=O] | 0.582 | 364.0 |
| 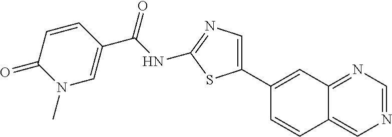 [SMILES: Cn1cc(C(=O)Nc2ncc(-c3ccc4cncnc4c3)s2)ccc1=O] | 0.534 | 364.0 |
| 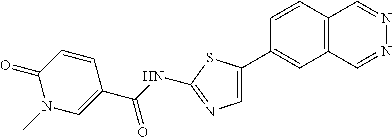 [SMILES: Cn1cc(C(=O)Nc2ncc(-c3ccc4cnncc4c3)s2)ccc1=O] | 0.494 | 364.0 |
| 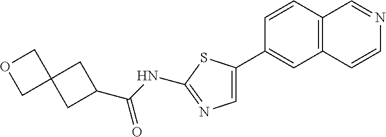 [SMILES: O=C(Nc1ncc(-c2ccc3cnccc3c2)s1)C1CC2(COC2)C1] | 0.435 | 352.2 |
| 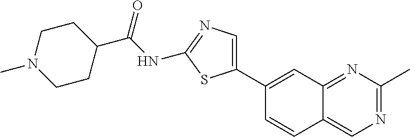 | 0.401 | 368.1 |
| 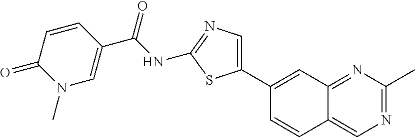 [SMILES: Cc1ncc2ccc(-c3cnc(NC(=O)c4ccc(=O)n(C)c4)s3)cc2n1] | 0.511 | 378.0 |
| 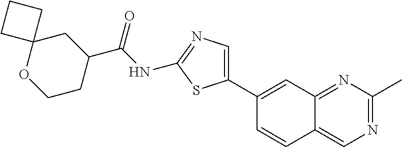 [SMILES: Cc1ncc2ccc(-c3cnc(NC(=O)C4CCOC5(CCC5)C4)s3)cc2n1] | 0.610 | 395.1 |
| 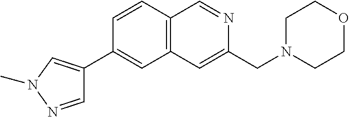 [SMILES: Cn1cc(-c2ccc3cnc(CN4CCOCC4)cc3c2)cn1] | 0.469 | 309.0 |
| 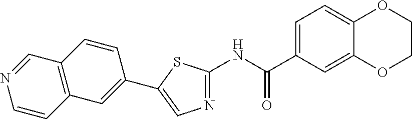 [SMILES: O=C(Nc1ncc(-c2ccc3cnccc3c2)s1)c1ccc2c(c1)OCCO2] | 0.645 | 390.0 |
| 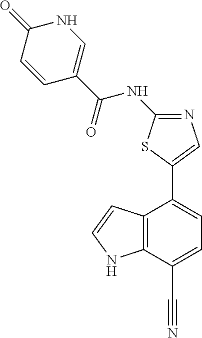 [SMILES: N#Cc1ccc(-c2cnc(NC(=O)c3ccc(=O)[nH]c3)s2)c2cc[nH]c12] | 0.59 | 362.2 |
| 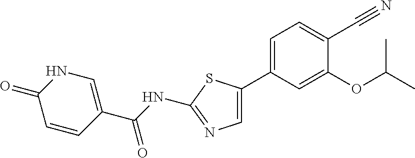 [SMILES: CC(C)Oc1cc(-c2cnc(NC(=O)c3ccc(=O)[nH]c3)s2)ccc1C#N] | 0.68 | 381.3 |
| 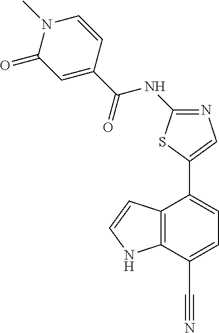 [SMILES: Cn1ccc(C(=O)Nc2ncc(-c3ccc(C#N)c4[nH]ccc34)s2)cc1=O] | 0.71 | 376.1 |
| 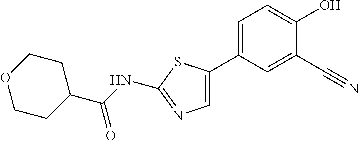 [SMILES: N#Cc1cc(-c2cnc(NC(=O)C3CCOCC3)s2)ccc1O] | 0.588 | 330.1 |
| 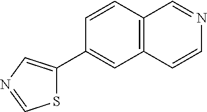 [SMILES: c1cc2cc(-c3cncs3)ccc2cn1] | 0.405 | 213.0 |
| 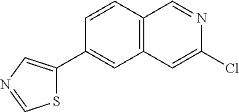 [SMILES: Clc1cc2cc(-c3cncs3)ccc2cn1] | 0.673 | 247.0 |
| 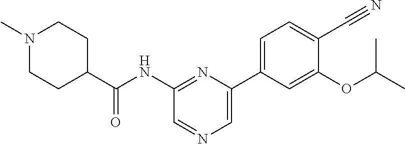 [SMILES: CC(C)Oc1cc(-c2cncc(NC(=O)C3CCN(C)CC3)n2)ccc1C#N] | 0.51 | 380.4 |
| 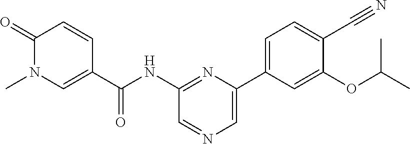 [SMILES: CC(C)Oc1cc(-c2cncc(NC(=O)c3ccc(=O)n(C)c3)n2)ccc1C#N] | 0.67 | 390.3 |
| 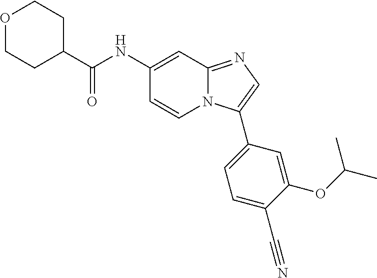 [SMILES: CC(C)Oc1cc(-c2cnc3cc(NC(=O)C4CCOCC4)ccn23)ccc1C#N] | 0.50 | 405.3 |
| 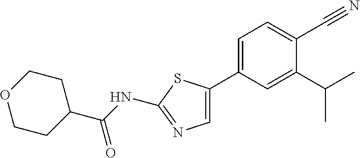 [SMILES: CC(C)c1cc(-c2cnc(NC(=O)C3CCOCC3)s2)ccc1C#N] | 0.82 | 356.2 |
| 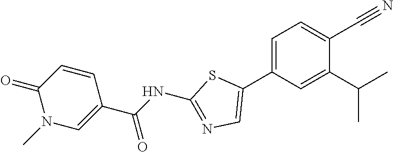 [SMILES: CC(C)c1cc(-c2cnc(NC(=O)c3ccc(=O)n(C)c3)s2)ccc1C#N] | 0.89 | 379.1 |
| 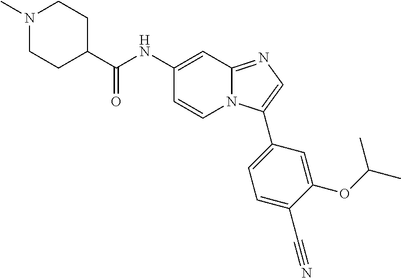 [SMILES: CC(C)Oc1cc(-c2cnc3cc(NC(=O)C4CCN(C)CC4)ccn23)ccc1C#N] | 0.44 | 418.3 |
| 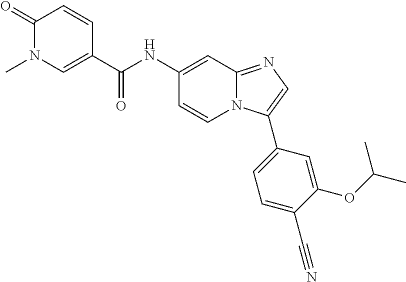 [SMILES: CC(C)Oc1cc(-c2cnc3cc(NC(=O)c4ccc(=O)n(C)c4)ccn23)ccc1C#N] | 0.56 | 428.2 |
| 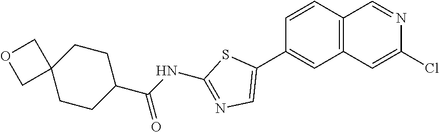 [SMILES: O=C(Nc1ncc(-c2ccc3cnc(Cl)cc3c2)s1)C1CCC2(CC1)COC2] | 0.855 | 414.1 |
| 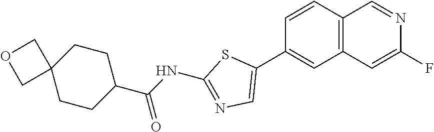 [SMILES: O=C(Nc1ncc(-c2ccc3cnc(F)cc3c2)s1)C1CCC2(CC1)COC2] | 0.818 | 398.1 |
| 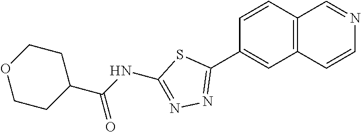 [SMILES: O=C(Nc1nnc(-c2ccc3cnccc3c2)s1)C1CCOCC1] | 0.643 | 341.3 |
| 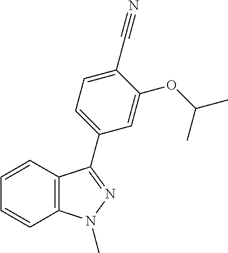 [SMILES: CC(C)Oc1cc(-c2nn(C)c3ccccc23)ccc1C#N] | 0.88 | 292.2 |
| 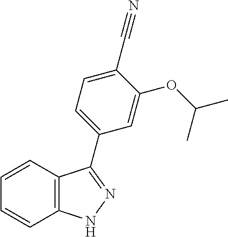 [SMILES: CC(C)Oc1cc(-c2n[nH]c3ccccc23)ccc1C#N] | 0.83 | 278.2 |
| 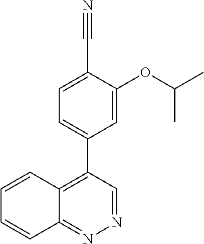 [SMILES: CC(C)Oc1cc(-c2cnnc3ccccc23)ccc1C#N] | 0.75 | 290.2 |
| 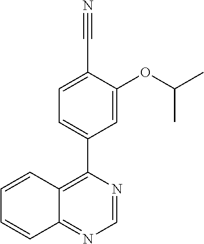 [SMILES: CC(C)Oc1cc(-c2ncnc3ccccc23)ccc1C#N] | 0.77 | 290.2 |
| 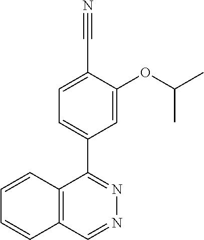 [SMILES: CC(C)Oc1cc(-c2nncc3ccccc23)ccc1C#N] | 0.68 | 290.3 |
| 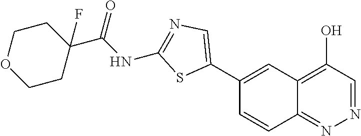 [SMILES: O=C(Nc1ncc(-c2ccc3nncc(O)c3c2)s1)C1(F)CCOCC1] | 0.578 | 375.0 |
| 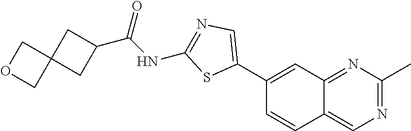 [SMILES: Cc1ncc2ccc(-c3cnc(NC(=O)C4CC5(COC5)C4)s3)cc2n1] | 0.475 | 367.3 |
| 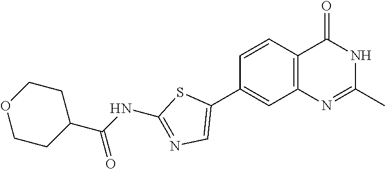 [SMILES: Cc1nc2cc(-c3cnc(NC(=O)C4CCOCC4)s3)ccc2c(=O)[nH]1] | 0.507 | 371.2 |
| 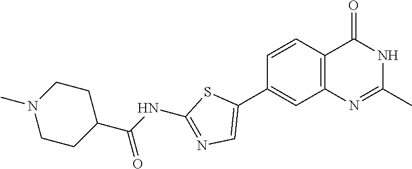 | 0.396 | 384.2 |
| 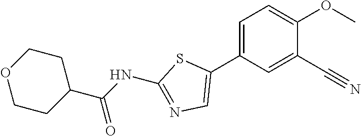 [SMILES: COc1ccc(-c2cnc(NC(=O)C3CCOCC3)s2)cc1C#N] | 0.750 | 344.2 |
| 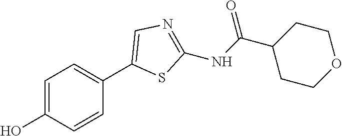 [SMILES: O=C(Nc1ncc(-c2ccc(O)cc2)s1)C1CCOCC1] | 0.578 | 305.1 |
| 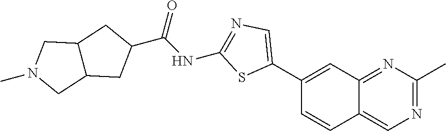 | 0.380 | 394.3 |
| 2-methyl-N-[5-(2-methylquinazolin-7-yl)-1,3-thiazol-2-yl]-3,3a,4,5,6,6a- | | |
| hexahydro-1H- | | |
| 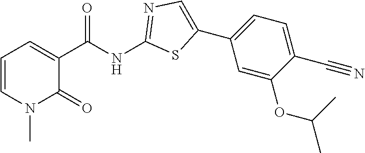 [SMILES: CC(C)Oc1cc(-c2cnc(NC(=O)c3cccn(C)c3=O)s2)ccc1C#N] | 0.85 | 395.2 |
| 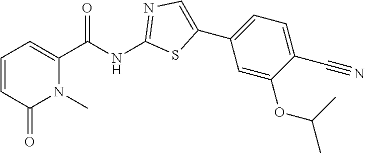 [SMILES: CC(C)Oc1cc(-c2cnc(NC(=O)c3cccc(=O)n3C)s2)ccc1C#N] | 0.77 | 395.2 |
| 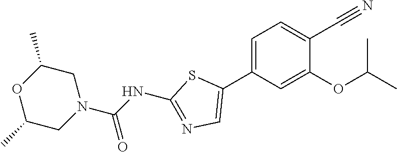 [SMILES: CC(C)Oc1cc(-c2cnc(NC(=O)N3C[C@@H](C)O[C@@H](C)C3)s2)ccc1C#N] | 0.83 | 401.1 |
| 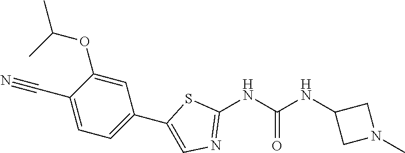 [SMILES: CC(C)Oc1cc(-c2cnc(NC(=O)NC3CN(C)C3)s2)ccc1C#N] | 0.57 | 372.0 |
| 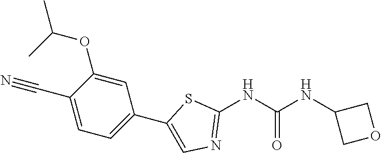 [SMILES: CC(C)Oc1cc(-c2cnc(NC(=O)NC3COC3)s2)ccc1C#N] | 0.72 | 359.0 |
| 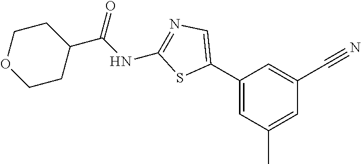 [SMILES: Cc1cc(C#N)cc(-c2cnc(NC(=O)C3CCOCC3)s2)c1] | 0.79 | 328.1 |
| 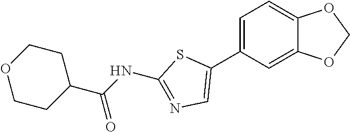 [SMILES: O=C(Nc1ncc(-c2ccc3c(c2)OCO3)s1)C1CCOCC1] | 0.726 | 333.1 |
| 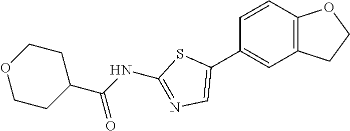 [SMILES: O=C(Nc1ncc(-c2ccc3c(c2)CCO3)s1)C1CCOCC1] | 0.733 | 331.1 |
| 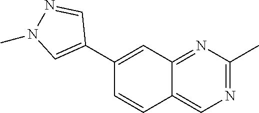 | 0.362 | 225.1 |
| 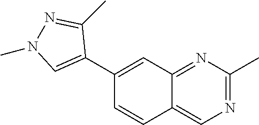 | 0.382 | 239.1 |
| 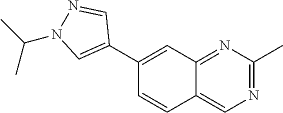 [SMILES: Cc1ncc2ccc(-c3cnn(C(C)C)c3)cc2n1] | 0.465 | 253.1 |
| 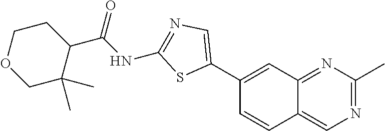 [SMILES: Cc1ncc2ccc(-c3cnc(NC(=O)C4CCOCC4(C)C)s3)cc2n1] | 0.618 | 383.3 |
| 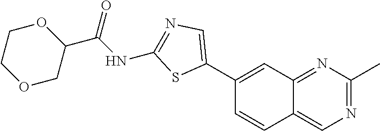 [SMILES: Cc1ncc2ccc(-c3cnc(NC(=O)C4COCCO4)s3)cc2n1] | 0.536 | 357.3 |
| 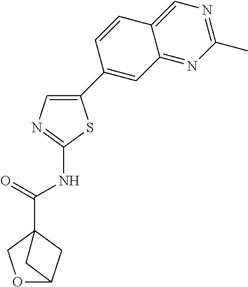 [SMILES: Cc1ncc2ccc(-c3cnc(NC(=O)C45COC(C4)C5)s3)cc2n1] | 0.533 | 353.3 |
| 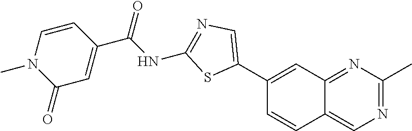 | 0.453 | 378.4 |
| 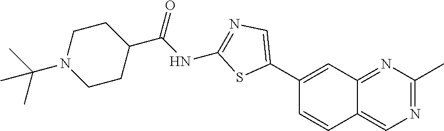 [SMILES: Cc1ncc2ccc(-c3cnc(NC(=O)C4CCN(C(C)(C)C)CC4)s3)cc2n1] | 0.521 | 410.5 |
| 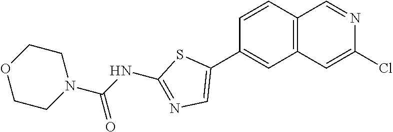 [SMILES: O=C(Nc1ncc(-c2ccc3cnc(Cl)cc3c2)s1)N1CCOCC1] | 0.747 | 375.1 |
| 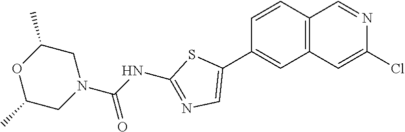 [SMILES: C[C@@H]1CN(C(=O)Nc2ncc(-c3ccc4cnc(Cl)cc4c3)s2)C[C@H](C)O1] | 0.849 | 403.1 |
| 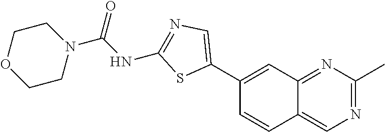 [SMILES: Cc1ncc2ccc(-c3cnc(NC(=O)N4CCOCC4)s3)cc2n1] | 0.428 | 356.1 |
| 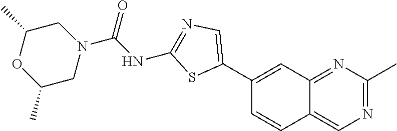 | 0.58 | 384.3 |
| 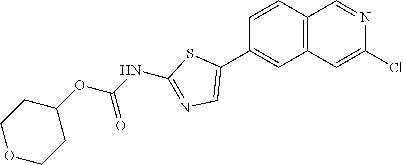 [SMILES: O=C(Nc1ncc(-c2ccc3cnc(Cl)cc3c2)s1)OC1CCOCC1] | 0.827 | 390.2 |
| 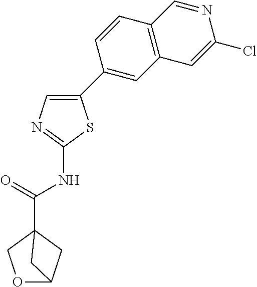 [SMILES: O=C(Nc1ncc(-c2ccc3cnc(Cl)cc3c2)s1)C12COC(C1)C2] | 0.780 | 372.1 |
| 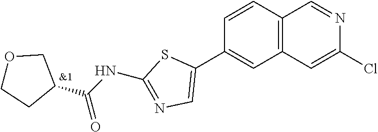 [SMILES: O=C(Nc1ncc(-c2ccc3cnc(Cl)cc3c2)s1)[C@@H]1CCOC1] | 0.768 | 360.1 |
| 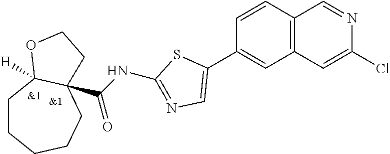 [SMILES: O=C(Nc1ncc(-c2ccc3cnc(Cl)cc3c2)s1)[C@@]12CCCCC[C@H]1OCC2] | 1.063 | 428.1 |
| 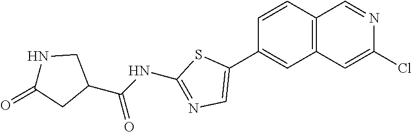 [SMILES: O=C1CC(C(=O)Nc2ncc(-c3ccc4cnc(Cl)cc4c3)s2)CN1] | 0.662 | 373.1 |
| 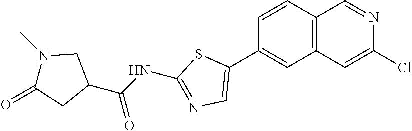 [SMILES: CN1CC(C(=O)Nc2ncc(-c3ccc4cnc(Cl)cc4c3)s2)CC1=O] | 0.704 | 387.1 |
| 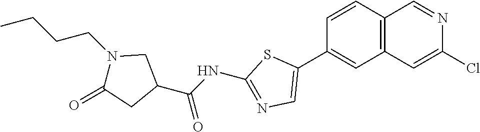 [SMILES: CCCCN1CC(C(=O)Nc2ncc(-c3ccc4cnc(Cl)cc4c3)s2)CC1=O] | 0.856 | 429.1 |
| 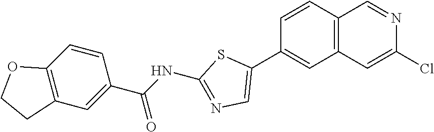 [SMILES: O=C(Nc1ncc(-c2ccc3cnc(Cl)cc3c2)s1)c1ccc2c(c1)CCO2] | 0.938 | 408.2 |
| 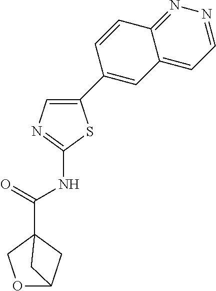 [SMILES: O=C(Nc1ncc(-c2ccc3nnccc3c2)s1)C12COC(C1)C2] | 0.555 | 339.1 |
| 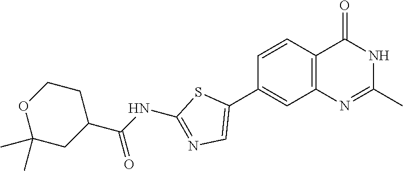 | 0.56 | 399.1 |
| 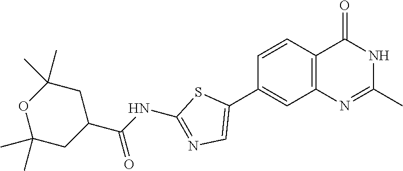 | 0.64 | 427.2 |
| 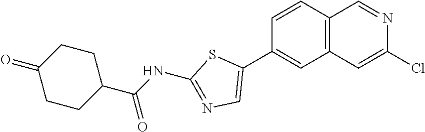 [SMILES: O=C1CCC(C(=O)Nc2ncc(-c3ccc4cnc(Cl)cc4c3)s2)CC1] | 0.790 | 386.1 |
| 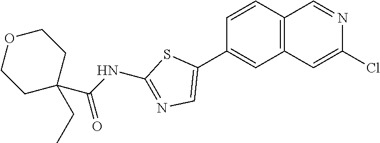 [SMILES: CCC1(C(=O)Nc2ncc(-c3ccc4cnc(Cl)cc4c3)s2)CCOCC1] | 0.898 | 402.1 |
| 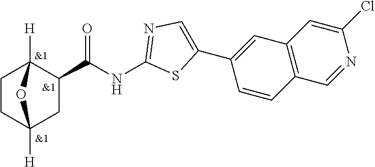 [SMILES: O=C(Nc1ncc(-c2ccc3cnc(Cl)cc3c2)s1)[C@H]1C[C@@H]2CC[C@H]1O2] | 0.807 | 386.1 |
| 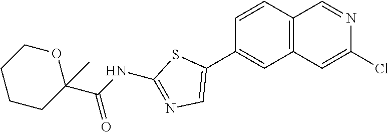 [SMILES: CC1(C(=O)Nc2ncc(-c3ccc4cnc(Cl)cc4c3)s2)CCCCO1] | 0.962 | 388.1 |
| 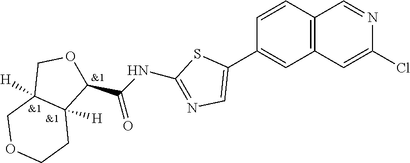 [SMILES: O=C(Nc1ncc(-c2ccc3cnc(Cl)cc3c2)s1)[C@@H]1OC[C@@H]2COCC[C@@H]21] | 0.811 | 416.1 |
| 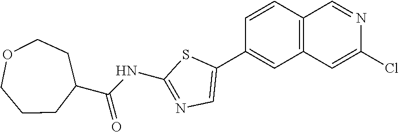 [SMILES: O=C(Nc1ncc(-c2ccc3cnc(Cl)cc3c2)s1)C1CCCOCC1] | 0.829 | 388.1 |
| 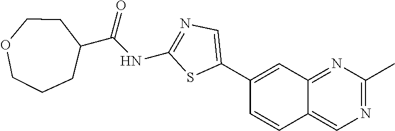 [SMILES: Cc1ncc2ccc(-c3cnc(NC(=O)C4CCCOCC4)s3)cc2n1] | 0.501 | 369.1 |
| 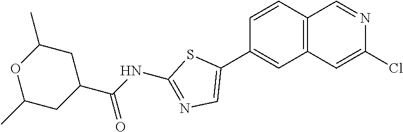 [SMILES: CC1CC(C(=O)Nc2ncc(-c3ccc4cnc(Cl)cc4c3)s2)CC(C)O1] | 0.866 | 402.3 |
| 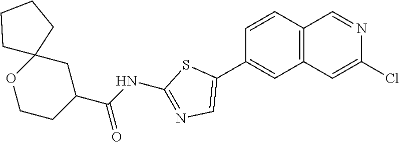 [SMILES: O=C(Nc1ncc(-c2ccc3cnc(Cl)cc3c2)s1)C1CCOC2(CCCC2)C1] | 0.933 | 428.3 |
| 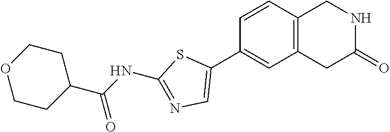 [SMILES: O=C1Cc2cc(-c3cnc(NC(=O)C4CCOCC4)s3)ccc2CN1] | 0.562 | 358.3 |
| 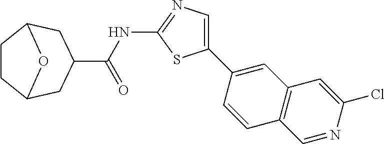 [SMILES: O=C(Nc1ncc(-c2ccc3cnc(Cl)cc3c2)s1)C1CC2CCC(C1)O2] | 0.823 | 400.1 |
| 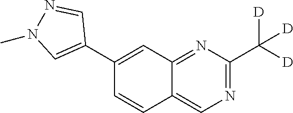 [SMILES: [2H]C([2H])([2H])c1ncc2ccc(-c3cnn(C)c3)cc2n1] | 0.475 | 228.3 |
| 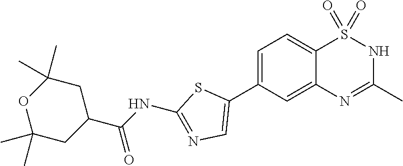 | 0.683 | 463.0 |
| 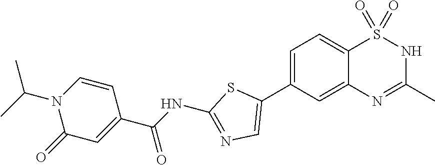 [SMILES: CC1=Nc2cc(-c3cnc(NC(=O)c4ccn(C(C)C)c(=O)c4)s3)ccc2S(=O)(=O)N1] | 0.370 | 458.1 |
| 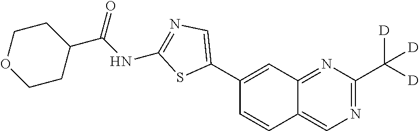 [SMILES: [2H]C([2H])([2H])c1ncc2ccc(-c3cnc(NC(=O)C4CCOCC4)s3)cc2n1] | 0.522 | 358.2 |
| 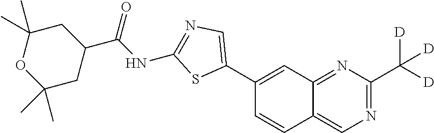 [SMILES: [2H]C([2H])([2H])c1ncc2ccc(-c3cnc(NC(=O)C4CC(C)(C)OC(C)(C)C4)s3)cc2n1] | 0.710 | 414.5 |
| 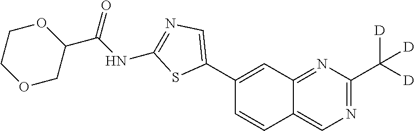 [SMILES: [2H]C([2H])([2H])c1ncc2ccc(-c3cnc(NC(=O)C4COCCO4)s3)cc2n1] | 0.524 | 360.2 |
| 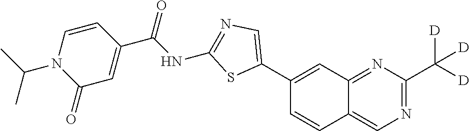 [SMILES: [2H]C([2H])([2H])c1ncc2ccc(-c3cnc(NC(=O)c4ccn(C(C)C)c(=O)c4)s3)cc2n1] | 0.506 | 409.2 |
| 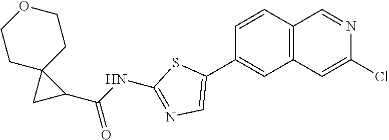 [SMILES: O=C(Nc1ncc(-c2ccc3cnc(Cl)cc3c2)s1)C1CC12CCOCC2] | 0.816 | 400.1 |
| 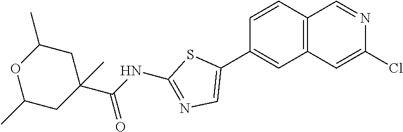 [SMILES: CC1CC(C)(C(=O)Nc2ncc(-c3ccc4cnc(Cl)cc4c3)s2)CC(C)O1] | 0.920 | 416.3 |
| 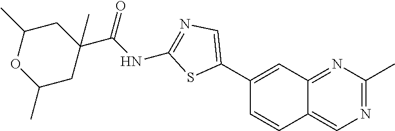 | 0.576 | 415.4 |
| 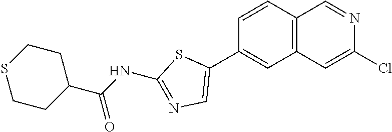 [SMILES: O=C(Nc1ncc(-c2ccc3cnc(Cl)cc3c2)s1)C1CCSCC1] | 0.832 | 389.9 |
| 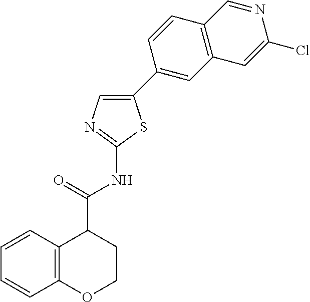 [SMILES: O=C(Nc1ncc(-c2ccc3cnc(Cl)cc3c2)s1)C1CCOc2ccccc21] | 0.912 | 422.2 |
| 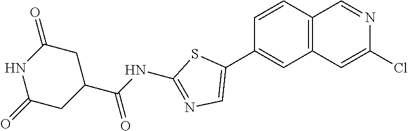 [SMILES: O=C1CC(C(=O)Nc2ncc(-c3ccc4cnc(Cl)cc4c3)s2)CC(=O)N1] | 0.669 | 401.3 |
| 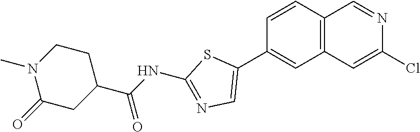 [SMILES: CN1CCC(C(=O)Nc2ncc(-c3ccc4cnc(Cl)cc4c3)s2)CC1=O] | 0.716 | 401.3 |
| 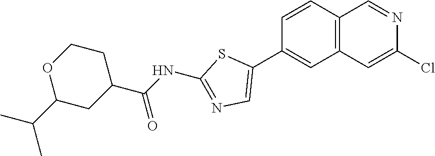 [SMILES: CC(C)C1CC(C(=O)Nc2ncc(-c3ccc4cnc(Cl)cc4c3)s2)CCO1] | 0.935 | 416.3 |
| 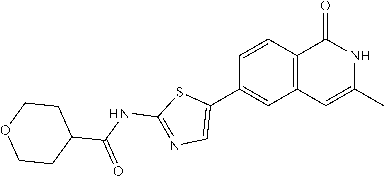 [SMILES: Cc1cc2cc(-c3cnc(NC(=O)C4CCOCC4)s3)ccc2c(=O)[nH]1] | 0.654 | 370.3 |
| 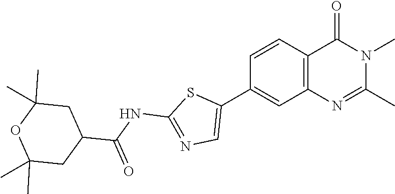 | 0.700 | 441.4 |
| 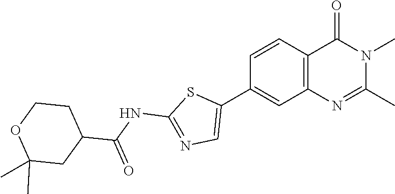 | 0.614 | 413.4 |
| 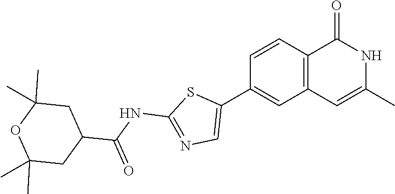 | 0.703 | 426.3 |
| 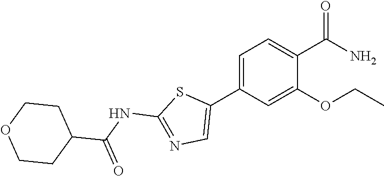 [SMILES: CCOc1cc(-c2cnc(NC(=O)C3CCOCC3)s2)ccc1C(N)=O] | 0.626 | 376.2 |
| 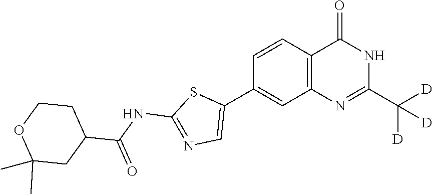 [SMILES: [2H]C([2H])([2H])c1nc2cc(-c3cnc(NC(=O)C4CCOC(C)(C)C4)s3)ccc2c(=O)[nH]1] | 0.56 | 402.1 |
| 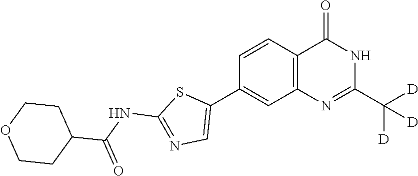 [SMILES: [2H]C([2H])([2H])c1nc2cc(-c3cnc(NC(=O)C4CCOCC4)s3)ccc2c(=O)[nH]1] | 0.49 | 374.3 |
| 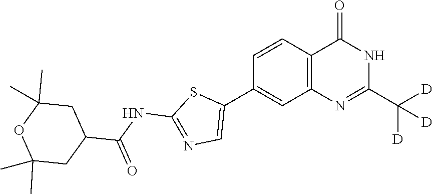 [SMILES: [2H]C([2H])([2H])c1nc2cc(-c3cnc(NC(=O)C4CC(C)(C)OC(C)(C)C4)s3)ccc2c(=O)[nH]1] | 0.65 | 430.2 |
| 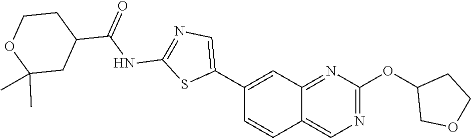 [SMILES: CC1(C)CC(C(=O)Nc2ncc(-c3ccc4cnc(OC5CCOC5)nc4c3)s2)CCO1] | 0.77 | 455.3 |
| 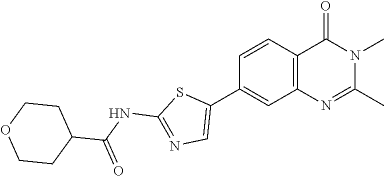 [SMILES: Cc1nc2cc(-c3cnc(NC(=O)C4CCOCC4)s3)ccc2c(=O)n1C] | 0.545 | 385.4 |
| 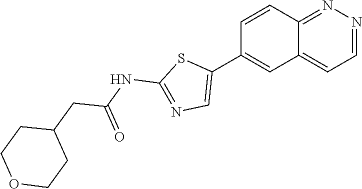 [SMILES: O=C(CC1CCOCC1)Nc1ncc(-c2ccc3nnccc3c2)s1] | 0.589 | 355.3 |
| 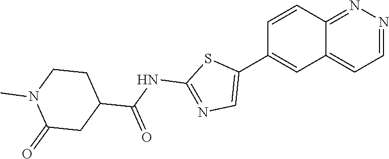 [SMILES: CN1CCC(C(=O)Nc2ncc(-c3ccc4nnccc4c3)s2)CC1=O] | 0.519 | 368.1 |
| 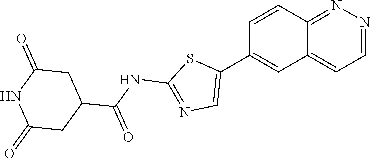 [SMILES: O=C1CC(C(=O)Nc2ncc(-c3ccc4nnccc4c3)s2)CC(=O)N1] | 0.470 | 368.1 |
| 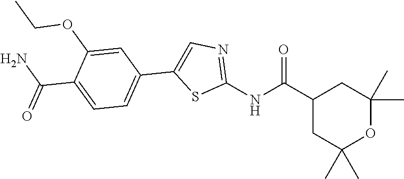 [SMILES: CCOc1cc(-c2cnc(NC(=O)C3CC(C)(C)OC(C)(C)C3)s2)ccc1C(N)=O] | 0.791 | 432.4 |
| 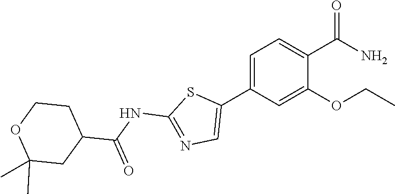 [SMILES: CCOc1cc(-c2cnc(NC(=O)C3CCOC(C)(C)C3)s2)ccc1C(N)=O] | 0.695 | 404.3 |
| 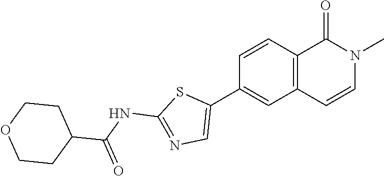 [SMILES: Cn1ccc2cc(-c3cnc(NC(=O)C4CCOCC4)s3)ccc2c1=O] | 0.646 | 370.2 |
| 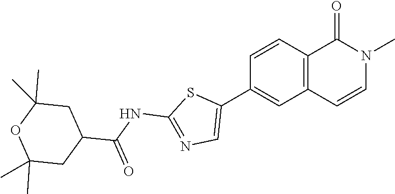 | 0.79 | 426.2 |
| 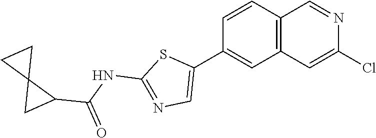 [SMILES: O=C(Nc1ncc(-c2ccc3cnc(Cl)cc3c2)s1)C1CC12CC2] | 0.873 | 356.1 |
| 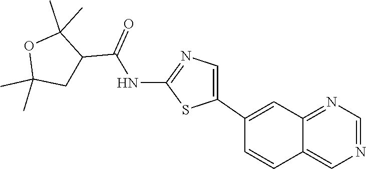 [SMILES: CC1(C)CC(C(=O)Nc2ncc(-c3ccc4cncnc4c3)s2)C(C)(C)O1] | 0.640 | 383.2 |
| 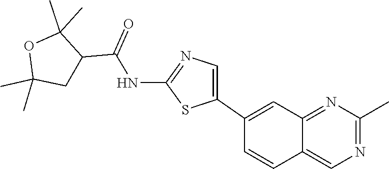 | 0.576 | 397.2 |
| 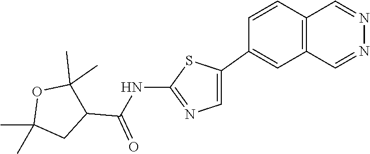 [SMILES: CC1(C)CC(C(=O)Nc2ncc(-c3ccc4cnncc4c3)s2)C(C)(C)O1] | 0.580 | 383.2 |
| 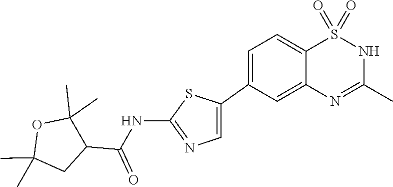 | 0.671 | 448.5 |
| 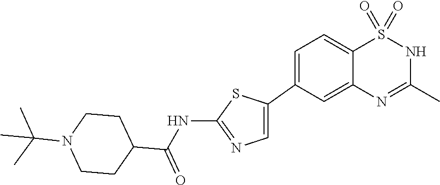 [SMILES: CC1=Nc2cc(-c3cnc(NC(=O)C4CCN(C(C)(C)C)CC4)s3)ccc2S(=O)(=O)N1] | 0.444 | 461.6 |
| 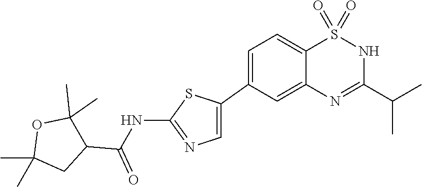 [SMILES: CC(C)C1=Nc2cc(-c3cnc(NC(=O)C4CC(C)(C)OC4(C)C)s3)ccc2S(=O)(=O)N1] | 0.758 | 477.2 |
| 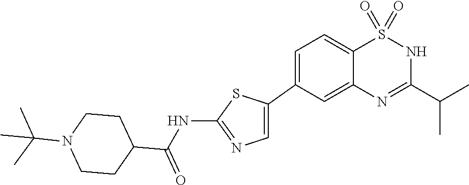 [SMILES: CC(C)C1=Nc2cc(-c3cnc(NC(=O)C4CCN(C(C)(C)C)CC4)s3)ccc2S(=O)(=O)N1] | 0.515 | 490.2 |
| 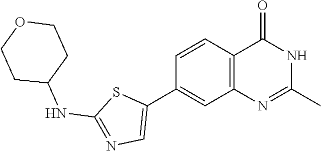 [SMILES: Cc1nc2cc(-c3cnc(NC4CCOCC4)s3)ccc2c(=O)[nH]1] | 0.40 | 343.0 |
| 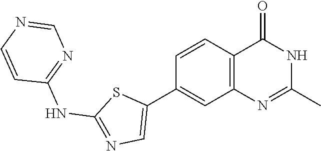 [SMILES: Cc1nc2cc(-c3cnc(Nc4ccncn4)s3)ccc2c(=O)[nH]1] | 0.40 | 337.1 |
| 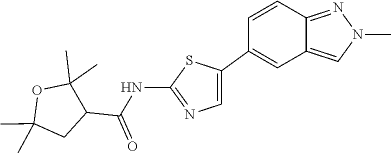 | 0.738 | 385.4 |
| 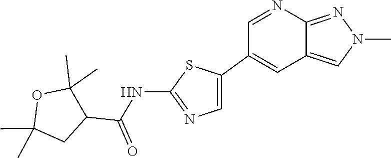 | 0.632 | 386.2 |
| 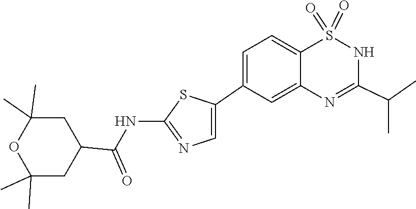 [SMILES: CC(C)C1=Nc2cc(-c3cnc(NC(=O)C4CC(C)(C)OC(C)(C)C4)s3)ccc2S(=O)(=O)N1] | 0.745 | 491.0 |
| 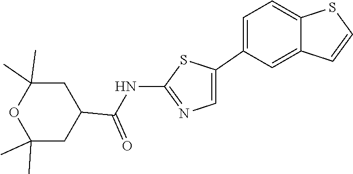 [SMILES: CC1(C)CC(C(=O)Nc2ncc(-c3ccc4sccc4c3)s2)CC(C)(C)O1] | 0.989 | 401.2 |
| 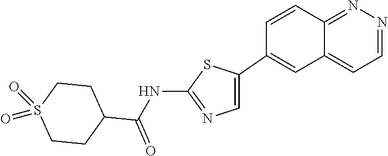 [SMILES: O=C(Nc1ncc(-c2ccc3nnccc3c2)s1)C1CCS(=O)(=O)CC1] | 0.508 | 389.3 |
| 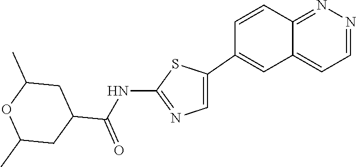 [SMILES: CC1CC(C(=O)Nc2ncc(-c3ccc4nnccc4c3)s2)CC(C)O1] | 0.633 | 369.4 |
| 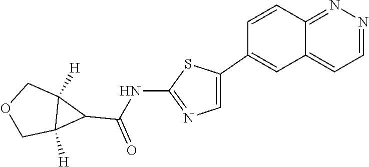 [SMILES: O=C(Nc1ncc(-c2ccc3nnccc3c2)s1)C1[C@H]2COC[C@@H]12] | 0.577 | 339.1 |
| 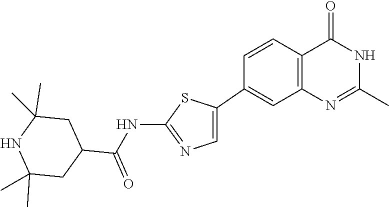 | 0.45 | 426.2 |
| 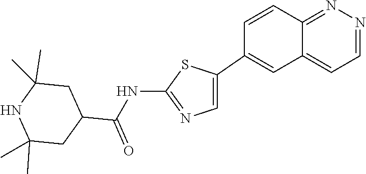 [SMILES: CC1(C)CC(C(=O)Nc2ncc(-c3ccc4nnccc4c3)s2)CC(C)(C)N1] | 0.479 | 396.2 |
| 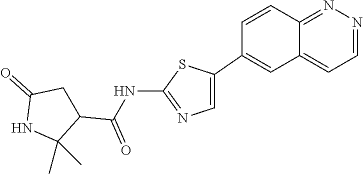 [SMILES: CC1(C)NC(=O)CC1C(=O)Nc1ncc(-c2ccc3nnccc3c2)s1] | 0.504 | 368.4 |
| 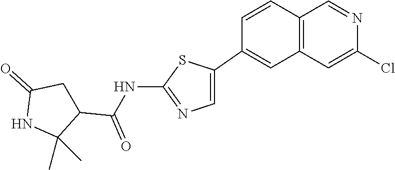 [SMILES: CC1(C)NC(=O)CC1C(=O)Nc1ncc(-c2ccc3cnc(Cl)cc3c2)s1] | 0.705 | 401.1 |
| 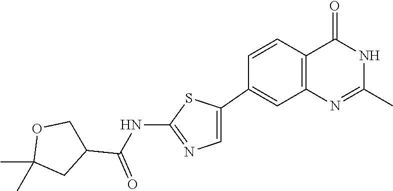 | 0.54 | 385.1 |
| 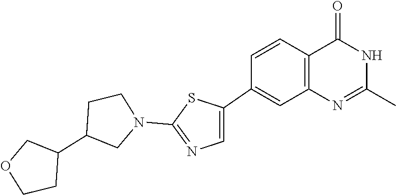 [SMILES: Cc1nc2cc(-c3cnc(N4CCC(C5CCOC5)C4)s3)ccc2c(=O)[nH]1] | 0.467 | 383.4 |
| 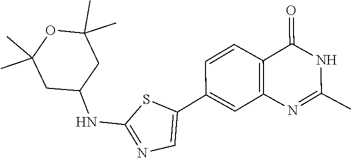 | 0.573 | 399.2 |
| 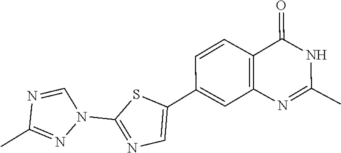 | 0.555 | 325.1 |
| 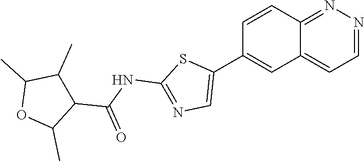 [SMILES: CC1OC(C)C(C(=O)Nc2ncc(-c3ccc4nnccc4c3)s2)C1C] | 0.646 | 369.4 |
| 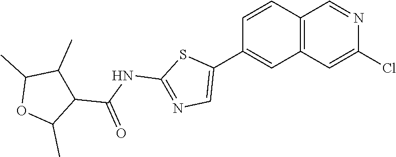 [SMILES: CC1OC(C)C(C(=O)Nc2ncc(-c3ccc4cnc(Cl)cc4c3)s2)C1C] | 0.842 | 402.4 |
| 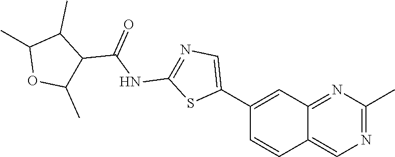 | 0.527 | 401.4 |
| 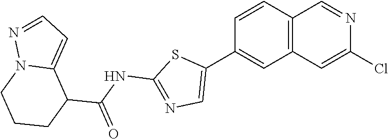 [SMILES: O=C(Nc1ncc(-c2ccc3cnc(Cl)cc3c2)s1)C1CCCn2nccc21] | 0.765 | 410.3 |
| 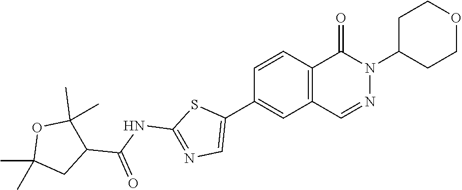 [SMILES: CC1(C)CC(C(=O)Nc2ncc(-c3ccc4c(=O)n(C5CCOCC5)ncc4c3)s2)C(C)(C)O1] | 0.96 | 483.2 |
| 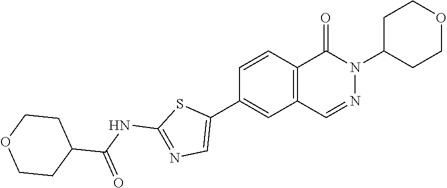 [SMILES: O=C(Nc1ncc(-c2ccc3c(=O)n(C4CCOCC4)ncc3c2)s1)C1CCOCC1] | 0.96 | 441.1 |
| 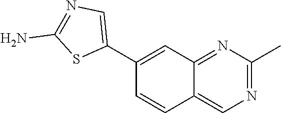 [SMILES: Cc1ncc2ccc(-c3cnc(N)s3)cc2n1] | 0.19 | 243.3 |
| 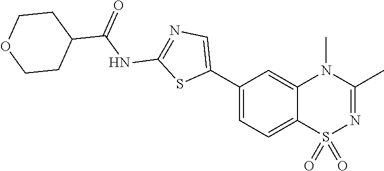 [SMILES: CC1=NS(=O)(=O)c2ccc(-c3cnc(NC(=O)C4CCOCC4)s3)cc2N1C] | 0.559 | 421.1 |
| 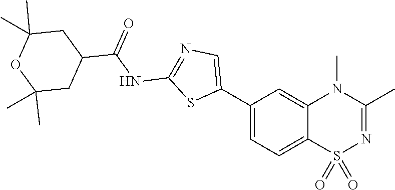 | 0.726 | 477.2 |
| 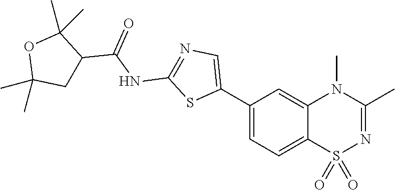 | 0.683 | 463.1 |
| 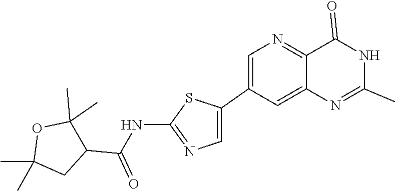 | 0.584 | 414.2 |
| 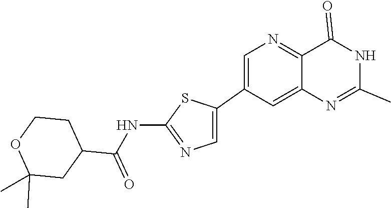 | 0.542 | 400.1 |
| 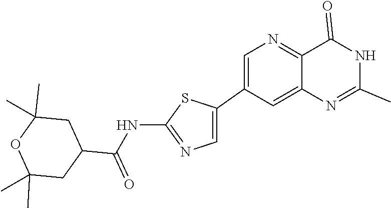 | 0.624 | 428.2 |
| 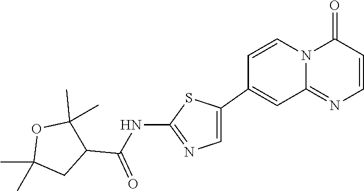 [SMILES: CC1(C)CC(C(=O)Nc2ncc(-c3ccn4c(=O)ccnc4c3)s2)C(C)(C)O1] | 0.54 | 399.4 |
| 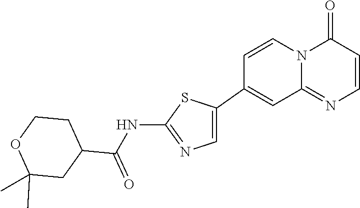 [SMILES: CC1(C)CC(C(=O)Nc2ncc(-c3ccn4c(=O)ccnc4c3)s2)CCO1] | 0.50 | 385.4 |
| 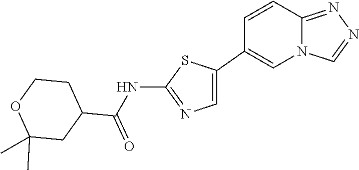 [SMILES: CC1(C)CC(C(=O)Nc2ncc(-c3ccc4nncn4c3)s2)CCO1] | 0.507 | 358.2 |
| 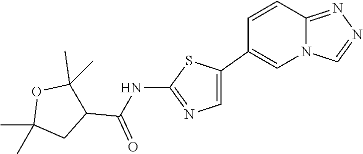 [SMILES: CC1(C)CC(C(=O)Nc2ncc(-c3ccc4nncn4c3)s2)C(C)(C)O1] | 0.555 | 372.2 |
| 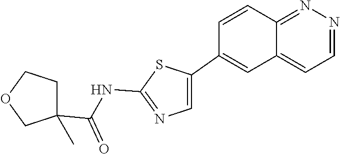 [SMILES: CC1(C(=O)Nc2ncc(-c3ccc4nnccc4c3)s2)CCOC1] | 0.574 | 341.1 |
| 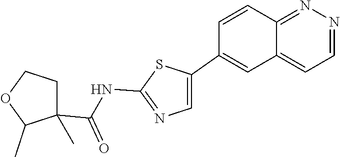 [SMILES: CC1OCCC1(C)C(=O)Nc1ncc(-c2ccc3nnccc3c2)s1] | 0.612 | 355.1 |
| 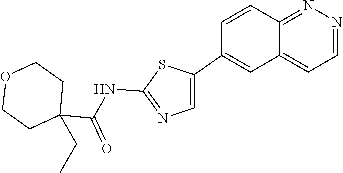 [SMILES: CCC1(C(=O)Nc2ncc(-c3ccc4nnccc4c3)s2)CCOCC1] | 0.635 | 369.2 |
| 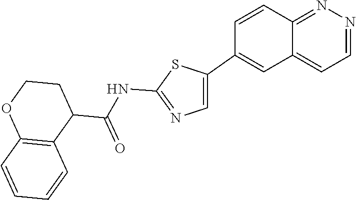 [SMILES: O=C(Nc1ncc(-c2ccc3nnccc3c2)s1)C1CCOc2ccccc21] | 0.693 | 389.1 |
| 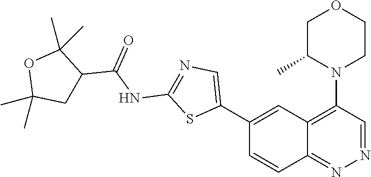 [SMILES: C[C@@H]1COCCN1c1cnnc2ccc(-c3cnc(NC(=O)C4CC(C)(C)OC4(C)C)s3)cc12] | 0.57 | 482.1 |
| 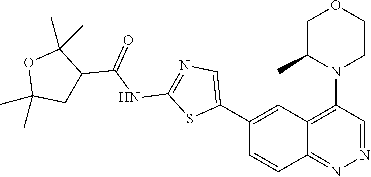 [SMILES: C[C@H]1COCCN1c1cnnc2ccc(-c3cnc(NC(=O)C4CC(C)(C)OC4(C)C)s3)cc12] | 0.56 | 482.1 |
| 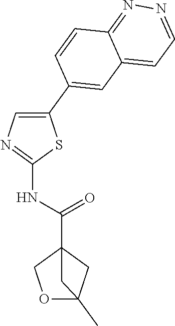 [SMILES: CC12CC(C(=O)Nc3ncc(-c4ccc5nnccc5c4)s3)(CO1)C2] | 0.577 | 353.1 |
| 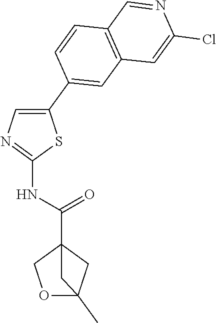 [SMILES: CC12CC(C(=O)Nc3ncc(-c4ccc5cnc(Cl)cc5c4)s3)(CO1)C2] | 0.778 | 386.1 |
| 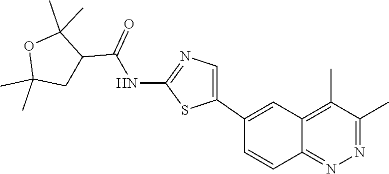 | 0.646 | 411.2 |
| 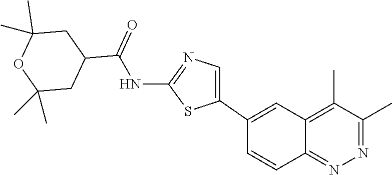 | 0.681 | 425.2 |
| 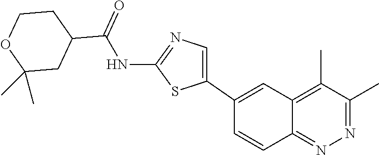 | 0.595 | 397.2 |
| 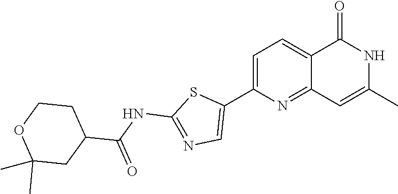 | 0.534 | 400.4 |
| 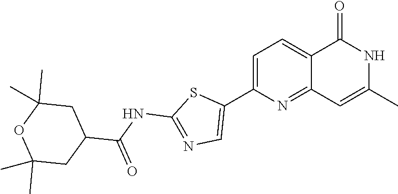 | 0.574 | 428.4 |
| 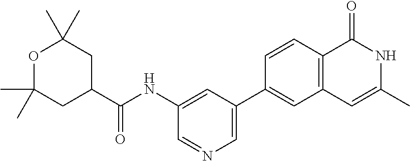 | 0.632 | 420.5 |
| 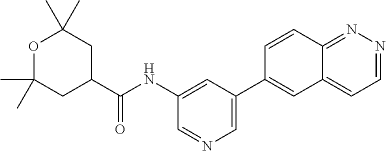 [SMILES: CC1(C)CC(C(=O)Nc2cncc(-c3ccc4nnccc4c3)c2)CC(C)(C)O1] | 0.609 | 391.5 |
| 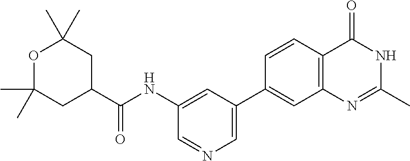 | 0.635 | 421.3 |

### DYRK1A Inhibition Assay

### Materials

### DYRK1A—Invitrogen #PV4105

Ser/Thr 18 peptide—Invitrogen #PR8227U
Phos-Ser/Thr 18 peptide—Invitrogen #PR8229U
Assay plate—PerkinElmer #6007279

### ATP—Sigma #A7699-5G

Development reagent A—Invitrogen #PR5194B
Development buffer—Invitrogen #PR4876B

### Compound Preparation

1. Test compounds were diluted to 1 mM in DMSO
2. Stock was further diluted 3-fold using Echo platform
3. 100 nL of DMSO into Columns 1 and 24 and 100 nL of compounds dilutions to Columns 2-23 in a plate.

### Assay Procedure

1. Add 5 μL enzyme & substrate mixture to each well in Column 2-23 and A24-H24 wells of the 384-well assay plate;
2. Add 5 μL 0% Phosphorylation control to A1-H1 and I24-P24 wells of the assay plate;
3. Add 5 μL 100% Phosphorylation control to I1-P1 wells of the assay plate;
4. Spin the assay plate (1000 rpm, 1 minute @23° C.);
5. Incubate enzyme with compounds for 15 minutes at 23° C.;
6. Add 5 μL ATP solution to each well of the assay plate;
7. Spin the plate (1000 rpm, 1 minute @23° C.);
8. Incubate the assay plate for 90 minutes at 23° C.;
9. Add 10 μL Development reagent A to each well of the assay plate;
10. Centrifuge the plate at 1000 rpm about 15 seconds and seal a film over assay plate. Incubate the assay plate for 30 minutes at 23° C.
11. Read assay plates on Envision (see Tables A, B, and C).

### Final Compound Concentrations

Assay buffer: 50 mM Hepes pH7.5, 10 mM MgCl₂, 1 mM EDTA, 0.01% Brij-35

### DYRK1A: 1 nM

### ATP: 20 μM

Ser/Thr 18 peptide: 2 μM
Reaction time: 90 minutes

TABLE A
DYRK1 Inhibition by Selected Compounds of Formula (I)

| Compound | DYRK1 |
|---|---|
| Number | IC₅₀ (nM) |
| 1 | B |
| 2 | A |
| 3 | B |
| 4 | B |
| 5 | B |
| 6 | C |
| 7 | C |
| 8 | B |
| 9 | A |
| 10 | |
| 11 | A |
| 12 | A |
| 13 | A |
| 14 | A |
| 15 | A |
| 16 | A |
| 17 | A |
| 18 | A |
| 19 | A |
| 20 | A |
| 21 | B |
| 22 | D |
| 23 | B |
| 24 | B |
| 25 | B |
| 26 | A |
| 27 | A |
| 28 | A |
| 29 | A |
| 30 | A |
| 31 | C |
| 32 | A |
| 33 | A |
| 34 | A |
| 35 | A |
| 36 | B |
| 37 | B |
| 38 | B |
| 39 | B |
| 40 | |
| 41 | A |
| 42 | A |
| 43 | |
| 44 | B |
| 45 | C |
| 46 | |
| 47 | C |
| 48 | C |
| 49 | |
| 50 | C |
| 51 | A |
| 52 | A |
| 53 | A |
| 54 | A |
| 55 | A |
| 56 | A |
| 57 | A |
| 58 | A |
| 59 | A |
| 60 | C |
| 61 | C |
| 62 | B |
| 63 | B |
| 64 | B |
| 65 | D |
| 66 | C |
| 67 | D |
| 68 | C |
| 69 | C |
| 70 | C |
| 71 | B |
| 72 | A |
| 73 | A |
| 74 | B |
| 75 | B |
| 76 | C |
| 77 | B |
| 78 | B |
| 79 | A |
| 80 | A |
| 81 | A |
| 82 | A |
| 83 | A |
| 84 | A |
| 85 | A |
| 86 | A |
| 87 | |
| 88 | C |
| 89 | B |
| 90 | |
| 91 | C |
| 92 | A |
| 93 | A |
| 94 | C |
| 95 | A |
| 96 | A |
| 97 | A |
| 98 | C |
| 99 | D |
| 100 | C |
| 101 | D |
| 102 | D |
| 103 | D |
| 104 | B |
| 105 | B |
| 106 | B |
| 107 | B |
| 108 | B |
| 109 | D |

(A ≤ 10 nM; 10 nM < B ≤ 100 nM; 100 nM < C ≤ 1 μM; D > 1 μM)

TABLE B
DYRK1 Inhibition by Selected Compounds of Formula (II)

| Compound | |
|---|---|
| Number | DYRK1 IC₅₀ |
| 1 | B |
| 2 | A |
| 3 | D |
| 4 | ND |
| 5 | C |
| 6 | D |
| 7 | D |
| 8 | A |
| 9 | B |
| 10 | B |
| 11 | B |
| 12 | B |
| 13 | B |
| 14 | C |
| 15 | B |
| 16 | A |
| 17 | A |
| 18 | B |
| 19 | A |
| 20 | A |
| 21 | A |
| 22 | ND |
| 23 | A |
| 24 | D |
| 25 | ND |
| 26 | ND |
| 27 | ND |
| 28 | A |
| 29 | B |
| 30 | ND |
| 31 | B |
| 32 | A |
| 33 | ND |
| 34 | A |
| 35 | B |
| 36 | ND |
| 37 | D |
| 38 | ND |
| 39 | C |
| 40 | D |
| 41 | ND |
| 42 | D |
| 43 | D |
| 44 | C |
| 45 | C |
| 46 | B |
| 47 | C |
| 48 | ND |
| 49 | D |
| 50 | ND |
| 51 | A |
| 52 | ND |
| 53 | A |
| 54 | A |
| 55 | D |
| 56 | A |
| 57 | A |
| 58 | B |
| 59 | C |

(A ≤ 100 nM; 100 nM < B ≤ 250 nM; 250 nM < C ≤ 1 μM; D > 1 μM)
ND = not determined

TABLE C
DYRK1 Inhibition by Selected Compounds of Formula (III)

| Compound | |
|---|---|
| Number | DYRK1 IC₅₀ (nM) |
| 1 | A |
| 2 | B |
| 3 | C |
| 4 | A |
| 5 | C |
| 6 | ND |
| 7 | ND |
| 8 | ND |
| 9 | ND |
| 10 | ND |
| 11 | D |
| 12 | D |
| 13 | A |
| 14 | D |
| 15 | D |
| 16 | D |
| 17 | D |
| 18 | D |
| 19 | D |
| 20 | D |
| 21 | D |
| 22 | D |
| 23 | D |
| 24 | ND |

(A ≤ 500 nM; 500 nM < B ≤ 1.5 μM; 1.5 μM < C ≤ 5 μM; D > 5 μM)

What is claimed is:

1. A compound of Formula (I): 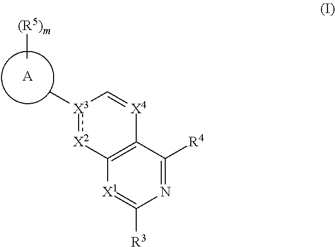
or a pharmaceutically acceptable salt thereof, wherein:
each dashed line represents a single bond or a double bond;
X¹ is CR¹ or N;
X² is CR², C(═O), or N;
X³ is C or N; provided that when X² is C(═O), X³ is N;
X⁴ is CH or N;
Ring A is phenyl or 5-6 membered heteroaryl;
R¹ is hydrogen, halogen, cyano, 3-10 membered heterocyclyl, C1-C6 alkyl optionally substituted with 3-6 membered heterocyclyl optionally substituted with —C(═O)C1-C6 alkyl or —C(═O)OR^{A}, C1-C6 alkoxy, —C(═O)-3-6 membered heterocyclyl optionally substituted with C1-C6 alkyl, or —OR^{B};
R^{B} is a 3-6 membered heterocyclyl, a 5-6 membered heteroaryl, a C6-C10 aryl, or a C3-C6 cycloalkyl each optionally substituted with 1-3 independently selected halogen, C1-C6 alkyl, C1-C6 haloalkyl, C1-C6 alkoxy, cyano, hydroxy, —C(═O)OH, —C(═O)C1-C6 alkyl, —S(O₂)—C1-C6 alkyl, or —NR^{C}R^{D};
R² is hydrogen, halogen, C1-C6 alkyl, C1-C6 alkoxy, —C(═O)-3-6 membered heterocyclyl, —NH—C3-C6 cycloalkyl-C(═O)OR^{A}, or —O—C3-C6 cycloalkyl-C(═O)OR^{A};
R³ is hydrogen, halogen, C1-C6 alkyl, cyano, C3-C6 cycloalkyl, —X—R^{G}, or 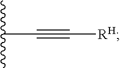
R⁴ is hydrogen or C1-C6 alkyl;
R⁵ is hydrogen, C1-C6 alkyl optionally substituted with 3-6 membered heterocyclyl; —X—R^{E}; —C3-C6 cycloalkyl-C(═O)OR^{A}; 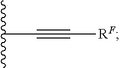
or
R⁵ and the carbon and/or nitrogen atom to which it is attached, forms a bond with an adjacent carbon or nitrogen atom, replacing the hydrogen atom on the adjacent carbon or nitrogen atom, and together R⁵ and the two adjacent carbon and/or nitrogen atoms in Ring A form a (i) C6-C10 aryl optionally substituted with a 3-10 membered heterocyclyl optionally substituted with 1-2 independently selected C1-C6 alkyl or —C(═O)OR′; (ii) a 3-6 membered heterocyclyl; or a (iii) a 5-6 membered heteroaryl optionally substituted with 1-2 substituents independently selected from C1-C6 alkyl, C1-C6 haloalkyl, C1-C6 alkoxy, 3-10 membered heterocyclyl optionally substituted with 1-2 independently selected C1-C6 alkyl or C(═O)OR′, and 5-6 membered heteroaryl optionally substituted with C1-C6 alkyl;
R^{E} is a 3-10 membered heterocyclyl, a 5-6 membered heteroaryl, or a C3-C6 cycloalkyl each optionally substituted with 1-3 independently selected C1-C6 alkyl optionally substituted with C1-C6 alkoxy, NR^{I}R^{J}, or —C(═O)OH;
R^{F} is a 3-6 membered heterocyclyl, a 5-6 membered heteroaryl, or a C3-C6 cycloalkyl each optionally substituted with C1-C6 alkyl; or a C2-C6 alkynyl optionally substituted with hydroxy;
R^{G} is a 3-6 membered heterocyclyl or a C3-C6 cycloalkyl each optionally substituted with 1-3 independently selected C1-C6 alkyl, —C(═O)C1-C6 alkyl, —C(═O)OH, or NR^{C}R^{D};
R^{H} is a C1-C6 alkyl optionally substituted with hydroxy or a 3-6 membered heterocyclyl optionally substituted with C1-C6 alkyl;
X is —NH—, —NH(C═O)—, —NHC(═O)O—, —O—, —(C═O)— or CH₂;
each R^{A}, R^{C}, R^{D}, R^{I}, and R^{J} is independently selected from hydrogen and C1-C6 alkyl; and
m is 0, 1, or 2.

2. The compound of claim 1, wherein one of X¹, X², X³, X⁴ is N, and each one of the remaining of X¹, X², X³, X⁴ is independently selected from C, C(═O), CH, CR¹ or CR².

3. The compound of claim 1 or 2, wherein X¹ is N; X² is CR²; X³ is C; and X⁴ is CH.

4. The compound of claim 1 or 2, wherein X² is N; X¹ is CR¹; X³ is C; and X⁴ is CH.

5. The compound of claim 1 or 2, wherein X³ is N; X¹ is CR¹; X² is C(═O); and X⁴ is CH.

6. The compound of claim 1 or 2, wherein X⁴ is N; X¹ is CR¹; X² is CR²; and X³ is C.

7. The compound of claim 1, wherein each one of X¹, X², X³, X⁴ is independently selected from C, C(═O), CH, CR¹ or CR².

8. The compound of claim 1, wherein ring A is a 5-membered heteroaryl.

9. The compound of any one of claims 1-8, wherein Ring A is 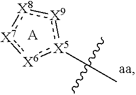
wherein aa represents the point of connection to X³, and each dash bond is independently a single bond or a double, and each one of X⁵, X⁶, X⁷, X⁸ and X⁹ is independently selected from C, (C═O), C═NH, CH, N, O, or S.

10. The compound of any one of claims 1-9, wherein Ring A is selected from the group consisting of thiazolyene, oxazolyene, imidazolyene, pyrazolyene, 1,2,4-triazolyene, 1,2,4-oxadiazolylene and 2-imine-thiazolylene.

11. The compound of any one of claims 1-9, wherein Ring A is selected from the group consisting of 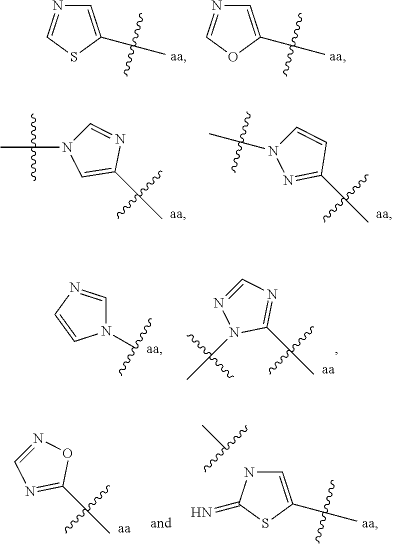
each of which is optionally substituted with one or two R⁵, and aa represents the point of attachment to X³ and the other wave line represents the point of connection to R⁵.

12. The compound of any one of claims 1-7, wherein Ring A is a 6-membered heteroaryl.

13. The compound of any one of claims 1-7 or 12, wherein Ring A is 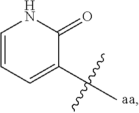
wherein aa represents the point of attachment to X³.

14. A compound of Formula (II): 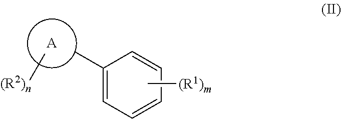
or a pharmaceutically acceptable salt thereof, wherein:
Ring A is a 5-14 membered heteroaryl or a 5-14 membered heterocyclyl;
each R¹ is independently halogen, hydroxyl, cyano, C1-C6 alkyl, C1-C6 alkoxy, —C(═O)OR^{A}, —NR^{B}R^{C}, and —C(═O)NR^{B}R^{C};
each R² is independently —C(═O)OR^{D}, C1-C6 alkyl, C2-C6 alkynyl optionally substituted with 4-8 membered heterocyclyl optionally substituted with C1-C6 alkyl, —C(═O)-phenyl, —(C1-C6 alkyl)-phenyl, —(C1-C6 alkyl)-4-10 membered heterocyclyl optionally substituted with C1-C6 alkyl, 4-10 membered heterocyclyl optionally substituted with C1-C6 alkyl or —CO₂C1-C6 alkyl, phenyl optionally substituted with cyano or fluoro, —NHC(═O)R^{E}, 5-6 membered heteroaryl optionally substituted with C1-C6 alkoxy,
m is 1, 2, or 3;
n is 0, 1, 2, or 3;
each R^{A}, R^{B}, R^{C}, and R^{D} is independently hydrogen or C1-C6 alkyl; and
each R^{E} is independently C3-C6 cycloalkyl, 4-8 membered heterocyclyl optionally substituted with C1-C6 alkyl, or 5-6 membered heteroaryl optionally substituted with C1-C6 alkyl.

15. The compound of claim 14, wherein Ring A is a 5-6 membered heteroaryl.

16. The compound of claim 14 or 15, wherein Ring A is thiazole or pyrazole.

17. The compound of claim 14 or 15, wherein Ring A is pyridine or pyrimidin-4(3H)-one.

18. The compound of claim 14, wherein Ring A is a bicyclic heteroaryl or a bicyclic heterocyclyl.

19. The compound of claim 14 or 18, wherein Ring A is pyrazolo[1,5-a]pyridine, 1H-pyrrolo[2,3-b]pyridine, pyrrolo[1,2-a]pyrazin-1(2H)-one, pyrazolo[1,5-a]pyrazine, imidazo[1,2-b]pyridazine, pyrazolo[1,5-a]pyrimidine, or 1,3-dihydro-2H-pyrrolo[2,3-b]pyridin-2-one.

20. The compound of claim 14 or 18, wherein Ring A is 5,6,7,8-tetrahydroimidazo[1,2-a]pyrazine, 1,3-dihydro-2H-pyrrolo[2,3-b]pyridin-2-one, or 1,3-dihydro-2H-imidazo[4,5-b]pyridin-2-one.

21. The compound of claim 14 or 18, wherein Ring A is 8H-pyrazolo[1,5-a]pyrrolo[3,2-e]pyrimidine.

22. The compound of claim 14 or 18, wherein Ring A is 7,8,9,10-tetrahydro-pyrazolo[5,1-f][1,6]naphthyridine, or 7,8-dihydro-6H-pyrazolo[1,5-a]pyrrolo[3,2-e]pyrimidine.

23. A compound of Formula (III): 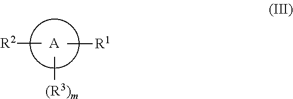
or a pharmaceutically acceptable salt thereof:
Ring A is 5-6 membered heteroaryl or 5-6 membered heterocyclyl;
R¹ is —NHC(═O)(C1-C6 alkylene)ₙR^{A}, phenyl optionally substituted with —NR^{F}R^{G}, -Q-R^{C}, or 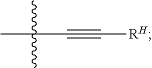
R² is C3-C6 cycloalkyl optionally substituted with —CO₂R^{B}, 5-10 membered heteroaryloxy, —(C1-C6 alkylene)ₚ-5-10 membered heteroaryl optionally substituted with C1-C6 alkyl, cyano, or 4-6 membered heterocyclyl; —(C1-C6 alkylene) t-phenyl optionally substituted with cyano or —NR^{D}R^{E}; 4-6 membered heterocyclyl optionally substituted with C1-C6 alkyl;
R³ is C1-C6 alkyl;
R^{A} is 4-6 membered heterocyclyl optionally substituted with C1-C6 alkyl, or 5-10 membered heteroaryl optionally substituted with C1-C6 alkoxy or C1-C6 alkyl,
R^{B} is hydrogen or C1-C6 alkyl;
R^{C} is 4-10 membered heterocyclyl, 5-10 membered heteroaryl, or phenyl optionally substituted with —(C1-C6 alkylene)-NR^{D}R^{E};
R^{D}, R^{E}, and R^{F} are independently hydrogen, C1-C6 alkyl, or C3-C6 cycloalkyl;
R^{G} is hydrogen, C1-C6 alkyl, —C(═O)—C1-C6 alkyl, or —C(═O)—C3-C6 cycloalkyl;
R^{H} is 4-6 membered heterocyclyl optionally substituted with 1-2 independently selected C1-C6 alkyl;
Q is C1-C6 alkylene, NH, or O;
m is 0 or 1;
n is 0 or 1;
p is 0 or 1; and
t is 0 or 1.

24. The compound of claim 23, wherein Ring A

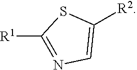

25. The compound of claim 23, wherein Ring A is

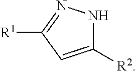

26. The compound of claim 23, wherein Ring A is

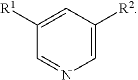

27. The compound of claim 23, wherein Ring A is

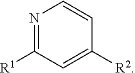

28. The compound of claim 23, wherein Ring A is

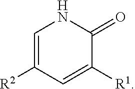

29. The compound of claim 23, wherein Ring A is

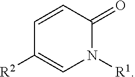

30. The compound of claim 23, wherein Ring A is

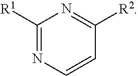

31. The compound of claim 23, wherein Ring A is

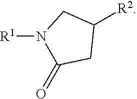

32. The compound of claim 23, wherein Ring A is

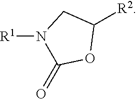

33. A compound selected from a compound in Table 1, Table 2, Table 3, or Table 4, or a pharmaceutically acceptable salt of any of the foregoing.

34. A pharmaceutical composition comprising a compound of any one of claims 1-33, or a pharmaceutically acceptable salt thereof, and pharmaceutically acceptable diluent or carrier.

35. A method for treating a neurological disorder in a subject in need thereof, the method comprising administering to the subject a therapeutically effective amount of a compound of any one of claims 1-33, or a pharmaceutically acceptable salt thereof, or a pharmaceutical composition according to claim 34.

36. The method of claim 35, wherein the neurological disorder is selected from the group consisting of Down Syndrome, Alzheimer's disease, and Alzheimer's disease associated with Down Syndrome.

37. The method of claim 35 or 36, wherein the neurological disorder is selected Alzheimer's disease associated with Down syndrome.

38. A method of treating a DYRK1A-associated neurological disorder in a subject, the method comprising administering to a subject identified or diagnosed as having a DYRK1A-associated neurological disorder a therapeutically effective amount of a compound of any one of claims 1-233, or a pharmaceutically acceptable salt thereof, or a pharmaceutical composition according to claim 34

39. A method for modulating DYRK1A in a mammalian cell, the method comprising contacting the mammalian cell with a therapeutically effective amount of a compound of any one of claims 1-33, or a pharmaceutically acceptable salt thereof.
